(19) United States
(12) Patent Application Publication
(10) Pub. No.: US 2002/0198362 A1
(43) Pub. Date: Dec. 26, 2002

(54) Compositions and methods for the detection, diagnosis and therapy of hematological malignancies

(76) Inventors: Alexander Gaiger, Seattle, WA (US); Paul A. Algate, Issaquah, WA (US); Jane Mannion, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/796,692

(22) Filed: Mar. 1, 2001

Related U.S. Application Data
(60) Provisional application No. 60/223,378, filed on Aug. 7, 2000.
(60) Provisional application No. 60/223,416, filed on Aug. 4, 2000.
(60) Provisional application No. 60/222,903, filed on Aug. 3, 2000.
(60) Provisional application No. 60/218,950, filed on Jul. 14, 2000.
(60) Provisional application No. 60/206,201, filed on May 22, 2000.
(60) Provisional application No. 60/202,084, filed on May 4, 2000.
(60) Provisional application No. 60/200,999, filed on May 1, 2000.
(60) Provisional application No. 60/200,303, filed on Apr. 28, 2000.
(60) Provisional application No. 60/200,779, filed on Apr. 28, 2000.
(60) Provisional application No. 60/200,545, filed on Apr. 27, 2000.
(60) Provisional application No. 60/190,479, filed on Mar. 17, 2000.
(60) Provisional application No. 60/186,126, filed on Mar. 1, 2000.

(51) Int. Cl. C07K 14/435; C12Q 1/68; C07H 21/04; C12P 21/02; C12N 5/06

(52) U.S. Cl. 530/350; 435/6; 435/69.1; 435/320.1; 435/325; 536/23.1

(57) ABSTRACT

Disclosed are methods and compositions for the detection, diagnosis, prognosis, and therapy of hematological malignancies, and in particular, human leukemias and lymphomas of the follicular, Hodgkin's and B cell and T cll non-Hodgkin's types. Disclosed are compositions, methods and kits for eliciting immune and T cell responses to specific malignancy-related antigenic polypeptides and antigenic polypeptide fragments thereof in an animal. Also disclosed are compositions and methods for use in the identification of cells and biological samples containing one or more hematological malignancy-related compositions, and methods for the detection and diagnosis of such diseases and affected cell types. Also disclosed are diagnostic and therapeutic kits, as well as methods for the diagnosis, therapy and/or prevention of a variety of leukemias and lymphomas.

The present application claims priority to U.S. Provisional Patent Applications Serial No. 60/186,126, filed Mar. 1, 2000; Serial No. 60/190,479, filed Mar. 17, 2000; Serial No. 60/200,545, filed Apr. 27, 2000; Serial No. 60/200,303, filed Apr. 28, 2000; Serial No. 60/200,779, filed Apr. 28, 2000; Serial No. 60/200,999; filed May 1, 2000; Serial No. 60/202,084, filed May 4, 2000; Serial No. 60/206,201, filed May 22, 2000; Serial No. 60/218,950, filed Jul. 14, 2000; Serial No. 60/222,903, filed Aug. 3, 2000; Serial No. 60/223,416, filed Aug. 4, 2000; and Serial No. 60/223,378, filed Aug. 7, 2000; the entire specification, claims and figures of each of which is specifically incorporated herein by reference in its entirety without disclaimer.

### BACKGROUND OF THE INVENTION

1. Field of the Invention

The present invention relates generally to the fields of cancer diagnosis and therapy. More particularly, it concerns the surprising discovery of compositions and methods for the detection and immunotherapy of hematological malignancies, and particularly, leukemias, and lymphomas of the follicular, Hodgkin's and T cell and B cell Non-Hodgkin's types. The invention provides new, effective methods, compositions and kits for eliciting immune and T-cell response to antigenic polypeptides, and antigenic peptide fragments isolated therefrom, and methods for the use of such compositions for diagnosis, detection, treatment, monitoring, and/or prevention of various types of human hematological malignancies. In particular, the invention provides polypeptide, peptide, antibody, antigen binding fragment, hybridoma, host cell, vector, and polynucleotide pompounds and compositions for use in identification and discrimination between various types of hematological malignancies, and methods for the detection, diagnosis, prognosis, monitoring, and therapy of such conditions in an affected animal.

2. Description of Related Art

Hematological Malignancies

Hematological malignancies, such as leukemias and lymphomas, are conditions characterized by abnormal growth and maturation of hematopoetic cells. Leukemias are generally neoplastic disorders of hematopoetic stem cells, and include adult and pediatric acute myeloid leukemia (AML), chronic myeloid leukemia (CML), acute lymphocytic leukemia (ALL), chronic lymphocytic leukemia (CLL) and secondary leukemia. Among lymphomas, there are two distinct groups: non-Hodgkin's lymphoma (NHL) and Hodgkin's disease. NHLs are the result of a clonal expansion of B- or T-cells, but the molecular pathogenesis of Hodgkin's disease, including lineage derivation and clonality, remains obscure. Other hematological malignancies include myelodysplastic syndromes (MDS), myeloproliferative syndromes (MPS) and myeloma. Hematological malignancies are generally serious disorders, resulting in a variety of symptoms, including bone marrow failure and organ failure.

NHLs are the sixth most common cause of cancer related deaths in the United States. Only prostate, breast, lung, colorectal and bladder cancer currently exceed lymphoma in annual incidence. In 1995, more than 45,000 new NHLs were diagnosed, and over 21,000 patients died of these diseases. The average age of lymphoma patients is relatively young (42 years), and the resulting number of years of life lost to these diseases renders NHLs fourth in economic impact among cancers in the United States. In the past 15 years, the American Cancer Society reported a 50% increase in the incidence of NHLs, one of the largest increases for any cancer group. Much of this increase has been attributed to the development of lymphomas in younger men who have acquired AIDS. Lymphomas are also the third most common childhood malignancy and account for approximately 10% of cancers in children. The survival rate (all ages) varies from 73% (low risk) to 26% (high risk).

3. Deficiencies in the Prior Art

Treatment for many hematological malignancies, including leukemias and lymphomas, remains difficult, and existing therapies are not universally effective. While treatments involving specific immunotherapy appear to have considerable potential, such treatments have been limited by the small number of known malignancy-associated antigens. Moreover the ability to detect such hematological malignancies in their early stages can be quite difficult depending upon the particular malady. The lack of a sufficient number of specific diagnostic and prognostic markers of the diseases, and identification of cells and tissues that can be affected, has significantly limited the field of oncology.

Accordingly, there remains a need in the art for improved methods for detecting, screening, diagnosis and treatment of hematological malignancies such as Hodgkin's disease, chronic lymphocytic leukemia, as well as follicular and non-Hodgkin's lymphomas. The present invention fulfills these and other inherent needs in the field, and provides significant advantages in the detection of cells, and cell types that express one or more polypeptides that have been shown to be over-expressed in one or more of such hematological malignancies.

### SUMMARY OF THE INVENTION

The present invention addresses the foregoing long-felt need and other deficiencies in the art by identifying new and effective strategies for the identification, detection, screening, diagnosis, prognosis, prophylaxis, therapy, and immunomodulation of one or more hematological malignancies, and in particular, leukemias such as chronic lymphocytic leukemia, and lymphomas, such as those of the follicular, Hodgkin's and non-Hodgkin's types.

The present invention is based, in part, upon the surprising and unexpected discovery that certain previously unknown or unidentified human polypeptides, peptides, and antigenic fragments derived therefrom have now been identified that are overexpressed in one or more types of hematological malignancies. The genes encoding several of these polypeptides are now identified and obtained in isolated form, and have been characterized using a series of molecular biology methodologies including subtractive library analysis, microarray screening, polynucleotide sequencing, peptide and epitopic identification and characterization, as well as expression profiling, and in vitro whole gene cell priming. A set of these polynucleotides, and the polypeptides, peptides, and antigenic fragments they encode are now identified and implicated in the complex processes of hematological malignancy disease onset, progression, and/or outcome, and in particular, diseases such as leukemias and lymphomas.

The inventors have further demonstrated that a number of these polynucleotides, and their encoded polypeptides, as well as antibodies, antigen presenting cells, T cells, and the antigen binding fragments derived from such antibodies are useful in the development of particularly advantageous compositions and methods for the detection, diagnosis, prognosis, prophylaxis and/or therapy of one or more of these diseases, and particularly those conditions that are characterized by (a) an increased, altered, elevated, or sustained expression of one or more polynucleotides that comprise at least a first sequence region that comprises a nucleic acid sequence as disclosed in any one of SEQ ID NO:1 through SEQ ID NO:278, or (b) an increased, altered, elevated, or sustained biological activity of one or more polypeptides that comprise at least a first sequence region that comprises an amino acid sequence as disclosed in any one of SEQ ID NO:669 through SEQ ID NO:2532.

The present invention also provides methods and uses for one or more of the disclosed peptide, polypeptide, antibody, antigen binding fragment, and polynucleotide compositions of the present invention in generating an immune response or in generating a T-cell response in an animal, and in particular in a mammal such as a human. The invention also provides methods and uses for one or more of these compositions in the identification, detection, and quantitation of hematological malignancy compositions in clinical samples, isolated cells, whole tissues, and even affected individuals. The compositions and methods disclosed herein also may be used in the preparation of one or more diagnostic reagents, assays, medicaments, or therapeutics, for diagnosis and/or therapy of such diseases.

In a first important embodiment, there is provided a composition comprising at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:669 to SEQ ID NO:2532. Exemplary preferred sequences are those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:669 to SEQ ID NO:2532, with those sequences that comprise at least a first coding region that comprises an amino acid sequence that is at least about 95%, about 96%, about 97%, about 98%, or about 99% identical to the amino acid sequence of any one of SEQ ID NO:669 to SEQ ID NO:2532 being examples of particularly preferred sequences in the practice of the present invention. Likewise, peptide and polypeptide compounds and compositions are also provided that comprise, consist essentially of, or consist of the amino acid sequence of any one of SEQ ID NO:669 to SEQ ID NO:2532.

In particular embodiments relating to compositions and methods for the detection, diagnosis, prognosis, prophylaxis, treatment, and therapy of Hodgkin's lymphoma, exemplary preferred peptide and polypeptide compositions have been provided herein. These include, but are not limited to, those peptide and polypeptide compounds and compositions that comprise at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:669 to SEQ ID NO:1380, and those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:669 to SEQ ID NO:1380, and even those sequences that comprise at least a first coding region that comprises an amino acid sequence that is at least about 95%, about 96%, about 97%, about 98%, or about 99% identical to the amino acid sequence of any one of SEQ ID NO:669 to SEQ ID NO:1380.

Likewise, in particular embodiments relating to compositions and methods for the detection, diagnosis, prognosis, prophylaxis, treatment, and therapy of follicular lymphoma, exemplary preferred peptide and polypeptide compositions have also been provided herein. These include, but are not limited to, those peptide and polypeptide compounds and compositions that comprise at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:1381 to SEQ ID NO:1859, and those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:1381 to SEQ ID NO:1859, and even those sequences that comprise at least a first coding region that comprises an amino acid sequence that is at least about 95%, about 96%, about 97%, about 98%, or about 99% identical to the amino acid sequence of any one of SEQ ID NO:1381 to SEQ ID NO:1859.

In a similar fashion, there are also embodiments disclosed herein that provide compositions and methods for the detection, diagnosis, prognosis, prophylaxis, treatment, and therapy of B cell non-Hodgkin's lymphoma. Exemplary preferred peptide and polypeptide compounds and compositions relating to this aspect of the invention include, but are not limited to, those petide and polypeptide compounds or compositions that comprise at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:1860 to SEQ ID NO:2105, and those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:1860 to SEQ ID NO:2105, and even those sequences that comprise at least a first coding region that comprises an amino acid sequence that is at least about 95%, about 96%, about 97%, about 98%, or about 99% identical to the amino acid sequence of any one of SEQ ID NO:1860 to SEQ ID NO:2105.

In those embodiments relating to compositions and methods for the detection, diagnosis, prognosis, prophylaxis, treatment, and therapy of T cell non-Hodgkin's lymphoma, exemplary preferred peptide and polypeptide compositions include those compositions that comprise at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:2106 to SEQ ID NO:2375, and those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:2106 to SEQ ID NO:2375, and even those sequences that comprise at least a first coding region that comprises an amino acid sequence that is at least about 95%, about 96%, about 97%, about 98%, or about 99% identical to the amino acid sequence of any one of SEQ ID NO:2106 to SEQ ID NO:2375.

In those embodiments relating to compositions and methods for the detection, diagnosis, prognosis, prophylaxis, treatment, and therapy of lymphoma, exemplary preferred peptide and polypeptide compositions include those compositions that comprise at least a first isolated peptide or polypeptide comprising at least a first isolated coding region that comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence of any one of SEQ ID NO:2376 to SEQ ID NO:2352, and those that comprise at least a first coding region that comprises an amino acid sequence that is at least about 85%, about 86%, about 87%, about 88%, about 89%, about 90%, about 91%, about 92%, about 93%, or about 94% identical to the amino acid sequence of any one of SEQ ID NO:2376 to SEQ ID NO:2352, and even those sequences that comprise at least a first coding region that comprises an amino acid sequence that is at least about 95%, about 96%, about 97%, about 98%, or about 99% identical to the amino acid sequence of any one of SEQ ID NO:2376 to SEQ ID NO:2352.

Exemplary peptides of the present invention may be of any suitable length, depending upon the particular application thereof, and encompass those peptides that are about 10, about 15, about 20, about 25, about 30, about 35, about 40, about 45, about 50, about 55, about 60, about 65, about 70, about 75, about 80, about 85, about 90, about 95, about 100 or so amino acids in length. Of course, the peptides of the invention may also encompass any intermediate lengths or integers within the stated ranges.

Exemplary polypeptides and proteins of the present invention may be of any suitable length, depending upon the particular application thereof, and encompass those polypeptides and proteins that are about 100, about 150, about 200, about 250, about 300, about 350, about 400, about 450, about 500, about 550, about 600, about 650, about 700, about 750, about 800, about 850, about 900, about 950, or about 1000 or so amino acids in length, as well as longer polypeptides and proteins that are about 1000, about 1050, about 1100, about 1200, about 1250, about 1300, about 1350, about 1400, about 1450, about 1500, about 1600, about 1700, about 1800, about 1900, about 2000, about 2500, about 3000, about 3500, about 4000, about 4500, or even about 5000 or so amino acids in length. Of course, the polypeptides and proteins of the invention may also encompass any intermediate lengths or integers within the stated ranges.

The peptides, polypeptides, proteins, antibodies, and antigen binding fragments of the present invention will preferably comprise at least a first isolated coding region that comprises a sequence of at least about 30, 35, 40, 45, 50, 55, 60, 65, 70, 75, 80, 85, 90, 95 or 100 contiguous amino acids from any one of SEQ ID NO:669 to SEQ ID NO:1380, SEQ ID NO:1381 to SEQ ID NO:1859, SEQ ID NO:1860 to SEQ ID NO:2105, SEQ ID NO:2106 to SEQ ID NO:2375 or SEQ ID NO:2376 to SEQ ID NO:2532.

Furthermore, the polypeptides, proteins, antibodies, and antigen binding fragments of the present invention will even more preferably comprise at least a first isolated coding region that comprises a sequence of at least about 100, 110, 120, 130, 140, 150, 160, 170, 180, 190, or 200 contiguous amino acids from any one of SEQ ID NO:669 to SEQ ID NO:1380, SEQ ID NO:1381 to SEQ ID NO:1859, SEQ ID NO:1860 to SEQ ID NO:2105, SEQ ID NO:2106 to SEQ ID NO:2375 or SEQ ID NO:2376 to SEQ ID NO:2532.

Likewise, the polypeptides, proteins, antibodies, and antigen binding fragments of the present invention may comprise at least a first isolated coding region that comprises a substantially longer sequence, such as for example, one of at least about 200, 220, 240, 260, 280, or 300 or more contiguous amino acids from any one of SEQ ID NO:669 to SEQ ID NO:1380, SEQ ID NO:1381 to SEQ ID NO:1859, SEQ ID NO:1860 to SEQ ID NO:2105, SEQ ID NO:2106 to SEQ ID NO:2375 or SEQ ID NO:2376 to SEQ ID NO:2532.

In illustrative embodiments, and particularly in those embodiments concerning methods and compositions relating to Hodgkin's lymphoma, the polypeptides of the invention comprise at least a first isolated coding region that (a) comprises, (b) consists essentially of, or (c) consists of, the amino acid sequence of SEQ ID NO:669, SEQ ID NO:670, SEQ ID NO:671, SEQ ID NO:672, SEQ ID NO:673, SEQ ID NO:674, SEQ ID NO:675, SEQ ID NO:676, SEQ ID NO:677, SEQ ID NO:678, SEQ ID NO:679, SEQ ID NO:680, SEQ ID NO:681, SEQ ID NO:682, SEQ ID NO:683, SEQ ID NO:684, SEQ ID NO:685, SEQ ID NO:686, SEQ ID NO:687, SEQ ID NO:688, SEQ ID NO:689, SEQ ID NO:690, SEQ ID NO:691, SEQ ID NO:692, SEQ ID NO:693, SEQ ID NO:694, SEQ ID NO:695, SEQ ID NO:696, SEQ ID NO:697, SEQ ID NO:698, SEQ ID NO:699, SEQ ID NO:700, SEQ ID NO:701, SEQ ID NO:702, SEQ ID NO:703, SEQ ID NO:704, SEQ ID NO:705, SEQ ID NO:706, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, SEQ ID NO:719, SEQ ID NO:720, SEQ ID NO:721, SEQ ID NO:722, SEQ ID NO:723, SEQ ID NO:724, SEQ ID NO:725, SEQ ID NO:726, SEQ ID NO:727, SEQ ID NO:728, SEQ ID NO:729, SEQ ID NO:730, SEQ ID NO:731, SEQ ID NO:732, SEQ ID NO:733, SEQ ID NO:734, SEQ ID NO:735, SEQ ID NO:736, SEQ ID NO:737, SEQ ID NO:738, SEQ ID NO:739, SEQ ID NO:740, SEQ ID NO:741, SEQ ID NO:742, SEQ ID NO:743, SEQ ID NO:744, SEQ ID NO:745, SEQ ID NO:746, SEQ ID NO:747, SEQ ID NO:748, SEQ ID NO:749, SEQ ID NO:750, SEQ ID NO:751, SEQ ID NO:752, SEQ ID NO:753, SEQ ID NO:754, SEQ ID NO:755, SEQ ID NO:756, SEQ ID NO:757, SEQ ID NO:758, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:780, SEQ ID NO:781, SEQ ID NO:782, SEQ ID NO:783, SEQ ID NO:784, SEQ ID NO:785, SEQ ID NO:786, SEQ ID NO:787, SEQ ID NO:788, SEQ ID NO:789, SEQ ID NO:790, SEQ ID NO:791, SEQ ID NO:792, SEQ ID NO:793, SEQ ID NO:794, SEQ ID NO:795, SEQ ID NO:796, SEQ ID NO:797, SEQ ID NO:798, SEQ ID NO:799, SEQ ID NO:800, SEQ ID NO:801, SEQ ID NO:802, SEQ ID NO:803, SEQ ID NO:804, SEQ ID NO:805, SEQ ID NO:806, SEQ ID NO:807, SEQ ID NO:808, SEQ ID NO:809, SEQ ID NO:810, SEQ ID NO:811, SEQ ID NO:812, SEQ ID NO:813, SEQ ID NO:814, SEQ ID NO:815, SEQ ID NO:816, SEQ ID NO:817, SEQ ID NO:818, SEQ ID NO:819, SEQ ID NO:820, SEQ ID NO:821, SEQ ID NO:822, SEQ ID NO:823, SEQ ID NO:824, SEQ ID NO:825, SEQ ID NO:826, SEQ ID NO:827, SEQ ID NO:828, SEQ ID NO:829, SEQ ID NO:830, SEQ ID NO:831, SEQ ID NO:832, SEQ ID NO:833, SEQ ID NO:834, SEQ ID NO:835, SEQ ID NO:836, SEQ ID NO:837, SEQ ID NO:838, SEQ ID NO:839, SEQ ID NO:840, SEQ ID NO:841, SEQ ID NO:842, SEQ ID NO:843, SEQ ID NO:844, SEQ ID NO:845, SEQ ID NO:846, SEQ ID NO:847, SEQ ID NO:848, SEQ ID NO:849, SEQ ID NO:850, SEQ ID NO:851, SEQ ID NO:852, SEQ ID NO:853, SEQ ID NO:854, SEQ ID NO:855, SEQ ID NO:856, SEQ ID NO:857, SEQ ID NO:858, SEQ ID NO:859, SEQ ID NO:860, SEQ ID NO:861, SEQ ID NO:862, SEQ ID NO:863, SEQ ID NO:864, SEQ ID NO:865, SEQ ID NO:866, SEQ ID NO:867, SEQ ID NO:868, SEQ ID NO:869, SEQ ID NO:870, SEQ ID NO:871, SEQ ID NO:872, SEQ ID NO:873, SEQ ID NO:874, SEQ ID NO:875, SEQ ID NO:876, SEQ ID NO:877, SEQ ID NO:878, SEQ ID NO:879, SEQ ID NO:880, SEQ ID NO:881, SEQ ID NO:882, SEQ ID NO:883, SEQ ID NO:884, SEQ ID NO:885, SEQ ID NO:886, SEQ ID NO:887, SEQ ID NO:888, SEQ ID NO:889, SEQ ID NO:890, SEQ ID NO:891, SEQ ID NO:892, SEQ ID NO:893, SEQ ID NO:894, SEQ ID NO:895, SEQ ID NO:896, SEQ ID NO:897, SEQ ID NO:898, SEQ ID NO:899, SEQ ID NO:900, SEQ ID NO:901, SEQ ID NO:902, SEQ ID NO:903, SEQ ID NO:904, SEQ ID NO:905, SEQ ID NO:906, SEQ ID NO:907, SEQ ID NO:908, SEQ ID NO:909, SEQ ID NO:910, SEQ ID NO:911, SEQ ID NO:912, SEQ ID NO:913, SEQ ID NO:914, SEQ ID NO:915, SEQ ID NO:916, SEQ ID NO:917, SEQ ID NO:918, SEQ ID NO:919, SEQ ID NO:920, SEQ ID NO:921, SEQ ID NO:922, SEQ ID NO:923, SEQ ID NO:924, SEQ ID NO:925, SEQ ID NO:926, SEQ ID NO:927, SEQ ID NO:928, SEQ ID NO:929, SEQ ID NO:930, SEQ ID NO:931, SEQ ID NO:932, SEQ ID NO:933, SEQ ID NO:934, SEQ ID NO:935, SEQ ID NO:936, SEQ ID NO:937, SEQ ID NO:938, SEQ ID NO:939, SEQ ID NO:940, SEQ ID NO:941, SEQ ID NO:942, SEQ ID NO:943, SEQ ID NO:944, SEQ ID NO:945, SEQ ID NO:946, SEQ ID NO:947, SEQ ID NO:948, SEQ ID NO:949, SEQ ID NO:950, SEQ ID NO:951, SEQ ID NO:952, SEQ ID NO:953, SEQ ID NO:954, SEQ ID NO:955, SEQ ID NO:956, SEQ ID NO:957, SEQ ID NO:958, SEQ ID NO:959, SEQ ID NO:960, SEQ ID NO:961, SEQ ID NO:962, SEQ ID NO:963, SEQ ID NO:964, SEQ ID NO:965, SEQ ID NO:966, SEQ ID NO:967, SEQ ID NO:968, SEQ ID NO:969, SEQ ID NO:970, SEQ ID NO:971, SEQ ID NO:972, SEQ ID NO:973, SEQ ID NO:974, SEQ ID NO:975, SEQ ID NO:976, SEQ ID NO:977, SEQ ID NO:978, SEQ ID NO:979, SEQ ID NO:980, SEQ ID NO:981, SEQ ID NO:982, SEQ ID NO:983, SEQ ID NO:984, SEQ ID NO:985, SEQ ID NO:986, SEQ ID NO:987, SEQ ID NO:988, SEQ ID NO:989, SEQ ID NO:990, SEQ ID NO:991, SEQ ID NO:992, SEQ ID NO:993, SEQ ID NO:994, SEQ ID NO:995, SEQ ID NO:996, SEQ ID NO:997, SEQ ID NO:998, SEQ ID NO:999, SEQ ID NO:1000, SEQ ID NO:1001, SEQ ID NO:1002, SEQ ID NO:1003, SEQ ID NO:1004, SEQ ID NO:1005, SEQ ID NO:1006, SEQ ID NO:1007, SEQ ID NO:1008, SEQ ID NO:1009, SEQ ID NO:1010, SEQ ID NO:1011, SEQ ID NO:1012, SEQ ID NO:1013, SEQ ID NO:1014, SEQ ID NO:1015, SEQ ID NO:1016, SEQ ID NO:1017, SEQ ID NO:1018, SEQ ID NO:1019, SEQ ID NO:1020, SEQ ID NO:1021, SEQ ID NO:1022, SEQ ID NO:1023, SEQ ID NO:1024, SEQ ID NO:1025, SEQ ID NO:1026, SEQ ID NO:1027, SEQ ID NO:1028, SEQ ID NO:1029, SEQ ID NO:1030, SEQ ID NO:1031, SEQ ID NO:1032, SEQ ID NO:1033, SEQ ID NO:1034, SEQ ID NO:1035, SEQ ID NO:1036, SEQ ID NO:1037, SEQ ID NO:1038, SEQ ID NO:1039, SEQ ID NO:1040, SEQ ID NO:1041, SEQ ID NO:1042, SEQ ID NO:1043, SEQ ID NO:1044, SEQ ID NO:1045, SEQ ID NO:1046, SEQ ID NO:1047, SEQ ID NO:1048, SEQ ID NO:1049, SEQ ID NO:1050, SEQ ID NO:1051, SEQ ID NO:1052, SEQ ID NO:1053, SEQ ID NO:1054, SEQ ID NO:1055, SEQ ID NO:1056, SEQ ID NO:1057, SEQ ID NO:1058, SEQ ID NO:1059, SEQ ID NO:1060, SEQ ID NO:1061, SEQ ID NO:1062, SEQ ID NO:1063, SEQ ID NO:1064, SEQ ID NO:1065, SEQ ID NO:1066, SEQ ID NO:1067, SEQ ID NO:1068, SEQ ID NO:1069, SEQ ID NO:1070, SEQ ID NO:1071, SEQ ID NO:1072, SEQ ID NO:1073, SEQ ID NO:1074, SEQ ID NO:1075, SEQ ID NO:1076, SEQ ID NO:1077, SEQ ID NO:1078, SEQ ID NO:1079, SEQ ID NO:1080, SEQ ID NO:1081, SEQ ID NO:1082, SEQ ID NO:1083, SEQ ID NO:1084, SEQ ID NO:1085, SEQ ID NO:1086, SEQ ID NO:1087, SEQ ID NO:1088, SEQ ID NO:1089, SEQ ID NO:1090, SEQ ID NO:1091, SEQ ID NO:1092, SEQ ID NO:1093, SEQ ID NO:1094, SEQ ID NO:1095, SEQ ID NO:1096, SEQ ID NO:1097, SEQ ID NO:1098, SEQ ID NO:1099, SEQ ID NO:1100, SEQ ID NO:1101, SEQ ID NO:1102, SEQ ID NO:1103, SEQ ID NO:1104, SEQ ID NO:1105, SEQ ID NO:1106, SEQ ID NO:1107, SEQ ID NO:1108, SEQ ID NO:1109, SEQ ID NO:1110, SEQ ID NO:1111, SEQ ID NO:1112, SEQ ID NO:1113, SEQ ID NO:1114, SEQ ID NO:1115, SEQ ID NO:1116, SEQ ID NO:1117, SEQ ID NO:1118, SEQ ID NO:1119, SEQ ID NO:1120, SEQ ID NO:1121, SEQ ID NO:1122, SEQ ID NO:1123, SEQ ID NO:1124, SEQ ID NO:1125, SEQ ID NO:1126, SEQ ID NO:1127, SEQ ID NO:1128, SEQ ID NO:1129, SEQ ID NO:1130, SEQ ID NO:1131, SEQ ID NO:1132, SEQ ID NO:1133, SEQ ID NO:1134, SEQ ID NO:1135, SEQ ID NO:1136, SEQ ID NO:1137, SEQ ID NO:1138, SEQ ID NO:1139, SEQ ID NO:1140, SEQ ID NO:1141, SEQ ID NO:1142, SEQ ID NO:1143, SEQ ID NO:1144, SEQ ID NO:1145, SEQ ID NO:1146, SEQ ID NO:1147, SEQ ID NO:1148, SEQ ID NO:1149, SEQ ID NO:1150, SEQ ID NO:1151, SEQ ID NO:1152, SEQ ID NO:1153, SEQ ID NO:1154, SEQ ID NO:1155, SEQ ID NO:1156, SEQ ID NO:1157, SEQ ID NO:1158, SEQ ID NO:1159, SEQ ID NO:1160, SEQ ID NO:1161, SEQ ID NO:1162, SEQ ID NO:1163, SEQ ID NO:1164, SEQ ID NO:1165, SEQ ID NO:1166, SEQ ID NO:1167, SEQ ID NO:1168, SEQ ID NO:1169, SEQ ID NO:1170, SEQ ID NO:1171, SEQ ID NO:1172, SEQ ID NO:1173, SEQ ID NO:1174, SEQ ID NO:1175, SEQ ID NO:1176, SEQ ID NO:1177, SEQ ID NO:11711, SEQ ID NO:1179, SEQ ID NO:1180, SEQ ID NO:1181, SEQ ID NO:1182, SEQ ID NO:1183, SEQ ID NO:1184, SEQ ID NO:1185, SEQ ID NO:1186, SEQ ID NO:1187, SEQ ID NO:1188, SEQ ID NO:1189, SEQ ID NO:1190, SEQ ID NO:1191, SEQ ID NO:1192, SEQ ID NO:1193, SEQ ID NO:1194, SEQ ID NO:1195, SEQ ID NO:1196, SEQ ID NO:1197, SEQ ID NO:1198, SEQ ID NO:1199, SEQ ID NO:1200, SEQ ID NO:1201, SEQ ID NO:1202, SEQ ID NO:1203, SEQ ID NO:1204, SEQ ID NO:1205, SEQ ID NO:1206, SEQ ID NO:1207, SEQ ID NO:1208, SEQ ID NO:1209, SEQ ID NO:1210, SEQ ID NO:1211, SEQ ID NO:1212, SEQ ID NO:1213, SEQ ID NO:1214, SEQ ID NO:1215, SEQ ID NO:1216, SEQ ID NO:1217, SEQ ID NO:1218, SEQ ID NO:1219, SEQ ID NO:1220, SEQ ID NO:1221, SEQ ID NO:1222, SEQ ID NO:1223, SEQ ID NO:1224, SEQ ID NO:1225, SEQ ID NO:1226, SEQ ID NO:1227, SEQ ID NO:1228, SEQ ID NO:1229, SEQ ID NO:1230, SEQ ID NO:1231, SEQ ID NO:1232, SEQ ID NO:1233, SEQ ID NO:1234, SEQ ID NO:1235, SEQ ID NO:1236, SEQ ID NO:1237, SEQ ID NO:1238, SEQ ID NO:1239, SEQ ID NO:1240, SEQ ID NO:1241, SEQ ID NO:1242, SEQ ID NO:1243, SEQ ID NO:1244, SEQ ID NO:1245, SEQ ID NO:1246, SEQ ID NO:1247, SEQ ID NO:1248, SEQ ID NO:1249, SEQ ID NO:1250, SEQ ID NO:1251, SEQ ID NO:1252, SEQ ID NO:1253, SEQ ID NO:1254, SEQ ID NO:1255, SEQ ID NO:1256, SEQ ID NO:1257, SEQ ID NO:1258, SEQ ID NO:1259, SEQ ID NO:1260, SEQ ID NO:1261, SEQ ID NO:1262, SEQ ID NO:1263, SEQ ID NO:1264, SEQ ID NO:1265, SEQ ID NO:1266, SEQ ID NO:1267, SEQ ID NO:1268, SEQ ID NO:1269, SEQ ID NO:1270, SEQ ID NO:1271, SEQ ID NO:1272, SEQ ID NO:1273, SEQ ID NO:1274, SEQ ID NO:1275, SEQ ID NO:1276, SEQ ID NO:1277, SEQ ID NO:1278, SEQ ID NO:1279, SEQ ID NO:1280, SEQ ID NO:1281, SEQ ID NO:1282, SEQ ID NO:1283, SEQ ID NO:1284, SEQ ID NO:1285, SEQ ID NO:1286, SEQ ID NO:1287, SEQ ID NO:1288, SEQ ID NO:1289, SEQ ID NO:1290, SEQ ID NO:1291, SEQ ID NO:1292, SEQ ID NO:1293, SEQ ID NO:1294, SEQ ID NO:1295, SEQ ID NO:1296, SEQ ID NO:1297, SEQ ID NO:1298, SEQ ID NO:1299, SEQ ID NO:1300, SEQ ID NO:1301, SEQ ID NO:1302, SEQ ID NO:1303, SEQ ID NO:1304, SEQ ID NO:1305, SEQ ID NO:1306, SEQ ID NO:1307, SEQ ID NO:1308, SEQ ID NO:1309, SEQ ID NO:1310, SEQ ID NO:1311, SEQ ID NO:1312, SEQ ID NO:1313, SEQ ID NO:1314, SEQ ID NO:1315, SEQ ID NO:1316, SEQ ID NO:1317, SEQ ID NO:1318, SEQ ID NO:1319, SEQ ID NO:1320, SEQ ID NO:1321, SEQ ID NO:1322, SEQ ID NO:1323, SEQ ID NO:1324, SEQ ID NO:1325, SEQ ID NO:1326, SEQ ID NO:1327, SEQ ID NO:1328, SEQ ID NO:1329, SEQ ID NO:1330, SEQ ID NO:1331, SEQ ID NO:1332, SEQ ID NO:1333, SEQ ID NO:1334, SEQ ID NO:1335, SEQ ID NO:1336, SEQ ID NO:1337, SEQ ID NO:1338, SEQ ID NO:1339, SEQ ID NO:1340, SEQ ID NO:1341, SEQ ID NO:1342, SEQ ID NO:1343, SEQ ID NO:1344, SEQ ID NO:1345, SEQ ID NO:1346, SEQ ID NO:1347, SEQ ID NO:1348, SEQ ID NO:1349, SEQ ID NO:1350, SEQ ID NO:1351, SEQ ID NO:1352, SEQ ID NO:1353, SEQ ID NO:1354, SEQ ID NO:1355, SEQ ID NO:1356, SEQ ID NO:1357, SEQ ID NO:1358, SEQ ID NO:1359, SEQ ID NO:1360, SEQ ID NO:1361, SEQ ID NO:1362, SEQ ID NO:1363, SEQ ID NO:1364, SEQ ID NO:1365, SEQ ID NO:1366, SEQ ID NO:1367, SEQ ID NO:1368, SEQ ID NO:1369, SEQ ID NO:1370, SEQ ID NO:1371, SEQ ID NO:1372, SEQ ID NO:1373, SEQ ID NO:1374, SEQ ID NO:1375, SEQ ID NO:1376, SEQ ID NO:1377, SEQ ID NO:1378, SEQ ID NO:1379, or SEQ ID NO:1380.

In illustrative embodiments, and particularly in those embodiments concerning methods and compositions relating to follicular lymphoma, the polypeptides of the invention comprise at least a first isolated coding region that (a) comprises, (b) consists essentially of, or (c) consists of, the amino acid sequence of SEQ ID NO:1381, SEQ ID NO:1382, SEQ ID NO:1383, SEQ ID NO:1384, SEQ ID NO:1385, SEQ ID NO:1386, SEQ ID NO:1387, SEQ ID NO:1388, SEQ ID NO:1389, SEQ ID NO:1390, SEQ ID NO:1391, SEQ ID NO:1392, SEQ ID NO:1393, SEQ ID NO:1394, SEQ ID NO:1395, SEQ ID NO:1396, SEQ ID NO:1397, SEQ ID NO:1398, SEQ ID NO:1399, SEQ ID NO:1400, SEQ ID NO:1401, SEQ ID NO:1402, SEQ ID NO:1403, SEQ ID NO:1404, SEQ ID NO:1405, SEQ ID NO:1406, SEQ ID NO:1407, SEQ ID NO:1408, SEQ ID NO:1409, SEQ ID NO:1410, SEQ ID NO:1411, SEQ ID NO:1412, SEQ ID NO:1413, SEQ ID NO:1414, SEQ ID NO:1415, SEQ ID NO:1416, SEQ ID NO:1417, SEQ ID NO:1418, SEQ ID NO:1419, SEQ ID NO:1420, SEQ ID NO:1421, SEQ ID NO:1422, SEQ ID NO:1423, SEQ ID NO:1424, SEQ ID NO:1425, SEQ ID NO:1426, SEQ ID NO:1427, SEQ ID NO:1428, SEQ ID NO:1429, SEQ ID NO:1430, SEQ ID NO:1431, SEQ ID NO:1432, SEQ ID NO:1433, SEQ ID NO:1434, SEQ ID NO:1435, SEQ ID NO:1436, SEQ ID NO:1437, SEQ ID NO:1438, SEQ ID NO:1439, SEQ ID NO:1440, SEQ ID NO:1441, SEQ ID NO:1442, SEQ ID NO:1443, SEQ ID NO:1444, SEQ ID NO:1445, SEQ ID NO:1446, SEQ ID NO:1447, SEQ ID NO:1448, SEQ ID NO:1449, SEQ ID NO:1450, SEQ ID NO:1451, SEQ ID NO:1452, SEQ ID NO:1453, SEQ ID NO:1454, SEQ ID NO:1455, SEQ ID NO:1456, SEQ ID NO:1457, SEQ ID NO:1458, SEQ ID NO:1459, SEQ ID NO:1460, SEQ ID NO:1461, SEQ ID NO:1462, SEQ ID NO:1463, SEQ ID NO:1464 , SEQ ID NO:1465, SEQ ID NO:1466, SEQ ID NO:1467, SEQ ID NO:1468, SEQ ID NO:1469, SEQ ID NO:1470, SEQ ID NO:1471, SEQ ID NO:1472, SEQ ID NO:1473, SEQ ID NO:1474, SEQ ID NO:1475, SEQ ID NO:1476, SEQ ID NO:1477, SEQ ID NO:1478, SEQ ID NO:1479, SEQ ID NO:1480, SEQ ID NO:1481, SEQ ID NO:1482, SEQ ID NO:1483, SEQ ID NO:1484, SEQ ID NO:1485, SEQ ID NO:1486, SEQ ID NO:1487, SEQ ID NO:1488, SEQ ID NO:1489, SEQ ID NO:1490, SEQ ID NO:1491, SEQ ID NO:1492, SEQ ID NO:1493, SEQ ID NO:1494, SEQ ID NO:1495, SEQ ID NO:1496, SEQ ID NO:1497, SEQ ID NO:1498, SEQ ID NO:1499, SEQ ID NO:1500, SEQ ID NO:1501, SEQ ID NO:1502, SEQ ID NO:1503, SEQ ID NO:1504, SEQ ID NO:1505, SEQ ID NO:1506, SEQ ID NO:1507, SEQ ID NO:1508, SEQ ID NO:1509, SEQ ID NO:1510, SEQ ID NO:1511, SEQ ID NO:1512, SEQ ID NO:1513, SEQ ID NO:1514, SEQ ID NO:1515, SEQ ID NO:1516, SEQ ID NO:1517, SEQ ID NO:1518, SEQ ID NO:1519, SEQ ID NO:1520, SEQ ID NO:1521 , SEQ ID NO:1522, SEQ ID NO:1523, SEQ ID NO:1524, SEQ ID NO:1525, SEQ ID NO:1526, SEQ ID NO:1527, SEQ ID NO:1528, SEQ ID NO:1529, SEQ ID NO:1530, SEQ ID NO:1531, SEQ ID NO:1532, SEQ ID NO:1533, SEQ ID NO:1534, SEQ ID NO:1535, SEQ ID NO:1536, SEQ ID NO:1537, SEQ ID NO:1538, SEQ ID NO:1539, SEQ ID NO:1540, SEQ ID NO:1541, SEQ ID NO:1542, SEQ ID NO:1543, SEQ ID NO:1544, SEQ ID NO:1545, SEQ ID NO:1546, SEQ ID NO:1547, SEQ ID NO:1548, SEQ ID NO:1549, SEQ ID NO:1550, SEQ ID NO:1551, SEQ ID NO:1552, SEQ ID NO:1553, SEQ ID NO:1554, SEQ ID NO:1555, SEQ ID NO:1556, SEQ ID NO:1557, SEQ ID NO:1558, SEQ ID NO:1559, SEQ ID NO:1560, SEQ ID NO:1561, SEQ ID NO:1562, SEQ ID NO:1563, SEQ ID NO:1564, SEQ ID NO:1565, SEQ ID NO:1566, SEQ ID NO:1567, SEQ ID NO:1568, SEQ ID NO:1569, SEQ ID NO:1570, SEQ ID NO:1571, SEQ ID NO:1572, SEQ ID NO:1573, SEQ ID NO:1574, SEQ ID NO:1575, SEQ ID NO:1576, SEQ ID NO:1577, SEQ ID NO:1578, SEQ ID NO:1579, SEQ ID NO:1580, SEQ ID NO:1581, SEQ ID NO:1582, SEQ ID NO:1583, SEQ ID NO:1584, SEQ ID NO:1585, SEQ ID NO:1586, SEQ ID NO:1587, SEQ ID NO:1588, SEQ ID NO:1589, SEQ ID NO:1590, SEQ ID NO:1591, SEQ ID NO:1592, SEQ ID NO:1593, SEQ ID NO:1594, SEQ ID NO:1595, SEQ ID NO:1596, SEQ ID NO:1597, SEQ ID NO:1598, SEQ ID NO:1599, SEQ ID NO:1600, SEQ ID NO:1601, SEQ ID NO:1602, SEQ ID NO:1603, SEQ ID NO:1604, SEQ ID NO:1605, SEQ ID NO:1606, SEQ ID NO:1607, SEQ ID NO:1608, SEQ ID NO:1609, SEQ ID NO:1610, SEQ ID NO:1611, SEQ ID NO:1612, SEQ ID NO:1613, SEQ ID NO:1614, SEQ ID NO:1615, SEQ ID NO:1616, SEQ ID NO:1617, SEQ ID NO:1618, SEQ ID NO:1619, SEQ ID NO:1620, SEQ ID NO:1621, SEQ ID NO:1622, SEQ ID NO:1623, SEQ ID NO:1624, SEQ ID NO:1625, SEQ ID NO:1626, SEQ ID NO:1627, SEQ ID NO:1628, SEQ ID NO:1629, SEQ ID NO:1630, SEQ ID NO:1631, SEQ ID NO:1632, SEQ ID NO:1633, SEQ ID NO:1634, SEQ ID NO:1635, SEQ ID NO:1636, SEQ ID NO:1637, SEQ ID NO:1638, SEQ ID NO:1639, SEQ ID NO:1640, SEQ ID NO:1641, SEQ ID NO:1642, SEQ ID NO:1643, SEQ ID NO:1644, SEQ ID NO:1645, SEQ ID NO:1646, SEQ ID NO:1647, SEQ ID NO:1648, SEQ ID NO:1649, SEQ ID NO:1650, SEQ ID NO:1651, SEQ ID NO:1652, SEQ ID NO:1653, SEQ ID NO:1654, SEQ ID NO:1655, SEQ ID NO:1656, SEQ ID NO:1657, SEQ ID NO:1658, SEQ ID NO:1659, SEQ ID NO:1660, SEQ ID NO:1661, SEQ ID NO:1662, SEQ ID NO:1663, SEQ ID NO:1664, SEQ ID NO:1665, SEQ ID NO:1666, SEQ ID NO:1667, SEQ ID NO:1668, SEQ ID NO:1669, SEQ ID NO:1670, SEQ ID NO:1671, SEQ ID NO:1672, SEQ ID NO:1673, SEQ ID NO:1674, SEQ ID NO:1675, SEQ ID NO:1676, SEQ ID NO:1677, SEQ ID NO:1678, SEQ ID NO:1679, SEQ ID NO:1680, SEQ ID NO:1681, SEQ ID NO:1682, SEQ ID NO:1683, SEQ ID NO:1684, SEQ ID NO:1685, SEQ ID NO:1686, SEQ ID NO:1687, SEQ ID NO:1688, SEQ ID NO:1689, SEQ ID NO:1690, SEQ ID NO:1691, SEQ ID NO:1692, SEQ ID NO:1693, SEQ ID NO:1694, SEQ ID NO:1695, SEQ ID NO:1696, SEQ ID NO:1697, SEQ ID NO:1698, SEQ ID NO:1699 SEQ ID NO:1700, SEQ ID NO:1701, SEQ ID NO:1702, SEQ ID NO:1703, SEQ ID NO:1704, SEQ ID NO:1705, SEQ ID NO:1706, SEQ ID NO:1707, SEQ ID NO:1708, SEQ ID NO:1709, SEQ ID NO:1710, SEQ ID NO:1711, SEQ ID NO:1712, SEQ ID NO:1713, SEQ ID NO:1714, SEQ ID NO:1715, SEQ ID NO:1716, SEQ ID NO:1717, SEQ ID NO:1718, SEQ ID NO:1719, SEQ ID NO:1720, SEQ ID NO:1721, SEQ ID NO:1722, SEQ ID NO:1723, SEQ ID NO:1724, SEQ ID NO:1725, SEQ ID NO:1726, SEQ ID NO:1727, SEQ ID NO:1728, SEQ ID NO:1729, SEQ ID NO:1730, SEQ ID NO:1731, SEQ ID NO:1732, SEQ ID NO:1733, SEQ ID NO:1734, SEQ ID NO:1735, SEQ ID NO:1736, SEQ ID NO:1737, SEQ ID NO:1738, SEQ ID NO:1739, SEQ ID NO:1740, SEQ ID NO:1741, SEQ ID NO:1742, SEQ ID NO:1743, SEQ ID NO:1744, SEQ ID NO:1745, SEQ ID NO:1746, SEQ ID NO:1747, SEQ ID NO:1748, SEQ ID NO:1749, SEQ ID NO:1750, SEQ ID NO:1751, SEQ ID NO:1752, SEQ ID NO:1753, SEQ ID NO:1754, SEQ ID NO:1755, SEQ ID NO:1756, SEQ ID NO:1757, SEQ ID NO:1758, SEQ ID NO:1759, SEQ ID NO:1760, SEQ ID NO:1761, SEQ ID NO:1762, SEQ ID NO:1763, SEQ ID NO:1764, SEQ ID NO:1765, SEQ ID NO:1766, SEQ ID NO:1767, SEQ ID NO:1768, SEQ ID NO:1769, SEQ ID NO:1770, SEQ ID NO:1771, SEQ ID NO:1772, SEQ ID NO:1773, SEQ ID NO:1774, SEQ ID NO:1775, SEQ ID NO:1776, SEQ ID NO:1777, SEQ ID NO:1778, SEQ ID NO:1779, SEQ ID NO:1780, SEQ ID NO:1781, SEQ ID NO:1782, SEQ ID NO:1783, SEQ ID NO:1784, SEQ ID NO:1785, SEQ ID NO:1786, SEQ ID NO:1787, SEQ ID NO:1788, SEQ ID NO:1789, SEQ ID NO:1790, SEQ ID NO:1791, SEQ ID NO:1792, SEQ ID NO:1793, SEQ ID NO:1794, SEQ ID NO:1795, SEQ ID NO:1796, SEQ ID NO:1797, SEQ ID NO:1798, SEQ ID NO:1799, SEQ ID NO:1800, SEQ ID NO:1801, SEQ ID NO:1802, SEQ ID NO:1803, SEQ ID NO:1804, SEQ ID NO:1805, SEQ ID NO:1806, SEQ ID NO:1807, SEQ ID NO:1808, SEQ ID NO:1809, SEQ ID NO:1810, SEQ ID NO:1811, SEQ ID NO:1812, SEQ ID NO:1813, SEQ ID NO:1814, SEQ ID NO:1815, SEQ ID NO:1816, SEQ ID NO:1817, SEQ ID NO:1818, SEQ ID NO:1819, SEQ ID NO:1820, SEQ ID NO:1821, SEQ ID NO:1822, SEQ ID NO:1823, SEQ ID NO:1824, SEQ ID NO:1825, SEQ ID NO:1826, SEQ ID NO:1827, SEQ ID NO:1828, SEQ ID NO:1829, SEQ ID NO:1830, SEQ ID NO:1831, SEQ ID NO:1832 , SEQ ID NO:1833, SEQ ID NO:1834, SEQ ID NO:1835, SEQ ID NO:1836, SEQ ID NO:1837, SEQ ID NO:1838, SEQ ID NO:1839, SEQ ID NO:1840, SEQ ID NO:1841, SEQ ID NO:1842, SEQ ID NO:1843 , SEQ ID NO:1844, SEQ ID NO:1845, SEQ ID NO:1846, SEQ ID NO:1847, SEQ ID NO:1848, SEQ ID NO:1849, SEQ ID NO:1850, SEQ ID NO:1851, SEQ ID NO:1852, SEQ ID NO:1853, SEQ ID NO:1854, SEQ ID NO:1855, SEQ ID NO:1856, SEQ ID NO:1857, SEQ ID NO:1858, or SEQ ID NO:1859.

In illustrative embodiments, and particularly in those embodiments concerning methods and compositions relating to B cell non-Hodgkin's lymphoma, the polypeptides of the invention comprise at least a first isolated coding region that (a) comprises, (b) consists essentially of, or (c) consists of, the amino acid sequence of SEQ ID NO:1860, SEQ ID NO:1861, SEQ ID NO:1862, SEQ ID NO:1863, SEQ ID NO:1864, SEQ ID NO:1865, SEQ ID NO:1866, SEQ ID NO:1867, SEQ ID NO:1868, SEQ ID NO:1869, SEQ ID NO:1870, SEQ ID NO:1871, SEQ ID NO:1872, SEQ ID NO:1873, SEQ ID NO:1874, SEQ ID NO:1875, SEQ ID NO:1876, SEQ ID NO:1877, SEQ ID NO:1878, SEQ ID NO:1879, SEQ ID NO:1880, SEQ ID NO:1881, SEQ ID NO:1882, SEQ ID NO:1883, SEQ ID NO:1884, SEQ ID NO:1885, SEQ ID NO:1886, SEQ ID NO:1887, SEQ ID NO:1888, SEQ ID NO:1889, SEQ ID NO:1890, SEQ ID NO:1891, SEQ ID NO:1892, SEQ ID NO:1893, SEQ ID NO:1894, SEQ ID NO:1895, SEQ ID NO:1896, SEQ ID NO:1897, SEQ ID NO:1898, SEQ ID NO:1899, SEQ ID NO:1900, SEQ ID NO:1901, SEQ ID NO:1902, SEQ ID NO:1903, SEQ ID NO:1904, SEQ ID NO:1905, SEQ ID NO:1906, SEQ ID NO:1907, SEQ ID NO:1908, SEQ ID NO:1909, SEQ ID NO:1910, SEQ ID NO:1911, SEQ ID NO:1912, SEQ ID NO:1913, SEQ ID NO:1914, SEQ ID NO:1915, SEQ ID NO:1916, SEQ ID NO:1917, SEQ ID NO:1918, SEQ ID NO:1919, SEQ ID NO:1920, SEQ ID NO:1921, SEQ ID NO:1922, SEQ ID NO:1923, SEQ ID NO:1924, SEQ ID NO:1925, SEQ ID NO:1926, SEQ ID NO:1927, SEQ ID NO:1928, SEQ ID NO:192 9, SEQ ID NO:1930, SEQ ID NO:1931, SEQ ID NO:1932 , SEQ ID NO:1933, SEQ ID NO:1934, SEQ ID NO:1935, SEQ ID NO:1936, SEQ ID NO:1937, SEQ ID NO:1938, SEQ ID NO:1939, SEQ ID NO:1940, SEQ ID NO:1941, SEQ ID NO:1942, SEQ ID NO:1943, SEQ ID NO:1944, SEQ ID NO:1945, SEQ ID NO:1946, SEQ ID NO:1947, SEQ ID NO:1948, SEQ ID NO:1949, SEQ ID NO:1950, SEQ ID NO:1951, SEQ ID NO:1952, SEQ ID NO:1953, SEQ ID NO:1954, SEQ ID NO:1955, SEQ ID NO:1956, SEQ ID NO:1957, SEQ ID NO:1958, SEQ ID NO:1959, SEQ ID NO:1960, SEQ ID NO:1961, SEQ ID NO:1962, SEQ ID NO:1963, SEQ ID NO:1964, SEQ ID NO:1965, SEQ ID NO:1966, SEQ ID NO:1967, SEQ ID NO:1968, SEQ ID NO:1969, SEQ ID NO:1970, SEQ ID NO:1971, SEQ ID NO:1972, SEQ ID NO:1973, SEQ ID NO:1974, SEQ ID NO:1975 , SEQ ID NO:1976, SEQ ID NO:1977, SEQ ID NO:1978, SEQ ID NO:1979, SEQ ID NO:1980, SEQ ID NO:1981, SEQ ID NO:1982, SEQ ID NO:1983, SEQ ID NO:1984, SEQ ID NO:1985, SEQ ID NO:1986, SEQ ID NO:1987, SEQ ID NO:1988, SEQ ID NO:1989, SEQ ID NO:1990, SEQ ID NO:1991, SEQ ID NO:1992, SEQ ID NO:1993, SEQ ID NO:1994, SEQ ID NO:1995, SEQ ID NO:1996, SEQ ID NO:1997, SEQ ID NO:1998, SEQ ID NO:1999, SEQ ID NO:2000, SEQ ID NO:2001, SEQ ID NO:2002, SEQ ID NO:2003, SEQ ID NO:2004, SEQ ID NO:2005, SEQ ID NO:2006, SEQ ID NO:2007, SEQ ID NO:2008, SEQ ID NO:2009, SEQ ID NO:2010, SEQ ID NO:2011, SEQ ID NO:2012, SEQ ID NO:2013, SEQ ID NO:2014, SEQ ID NO:2015, SEQ ID NO:2016, SEQ ID NO:2017, SEQ ID NO:2018, SEQ ID NO:2019, SEQ ID NO:2020, SEQ ID NO:2021, SEQ ID NO:2022, SEQ ID NO:2023, SEQ ID NO:2024, SEQ ID NO:2025, SEQ ID NO:2026, SEQ ID NO:2027, SEQ ID NO:2028, SEQ ID NO:2029, SEQ ID NO:2030, SEQ ID NO:2031, SEQ ID NO:2032, SEQ ID NO:2033, SEQ ID NO:2034, SEQ ID NO:2035, SEQ ID NO:2036, SEQ ID NO:2037, SEQ ID NO:2038, SEQ ID NO:2039, SEQ ID NO:2040, SEQ ID NO:2041, SEQ ID NO:2042, SEQ ID NO:2043, SEQ ID NO:2044, SEQ ID NO:2045, SEQ ID NO:2046, SEQ ID NO:2047, SEQ ID NO:2048, SEQ ID NO:2049, SEQ ID NO:2050, SEQ ID NO:2051, SEQ ID NO:2052, SEQ ID NO:2053, SEQ ID NO:2054, SEQ ID NO:2055, SEQ ID NO:2056, SEQ ID NO:2057, SEQ ID NO:2058, SEQ ID NO:2059, SEQ ID NO:2060, SEQ ID NO:2061, SEQ ID NO:2062, SEQ ID NO:2063, SEQ ID NO:2064, SEQ ID NO:2065, SEQ ID NO:2066, SEQ ID NO:2067, SEQ ID NO:2068, SEQ ID NO:2069, SEQ ID NO:2070, SEQ ID NO:2071, SEQ ID NO:2072, SEQ ID NO:2073, SEQ ID NO:2074, SEQ ID NO:2075, SEQ ID NO:2076, SEQ ID NO:2077, SEQ ID NO:2078, SEQ ID NO:2079, SEQ ID NO:2080, SEQ ID NO:2081, SEQ ID NO:2082, SEQ ID NO:2083, SEQ ID NO:2084, SEQ ID NO:2085, SEQ ID NO:2086, SEQ ID NO:2087, SEQ ID NO:2088, SEQ ID NO:2089, SEQ ID NO:2090, SEQ ID NO:2091, SEQ ID NO:2092, SEQ ID NO:2093, SEQ ID NO:2094, SEQ ID NO:2095, SEQ ID NO:2096 , SEQ ID NO:2097, SEQ ID NO:2098, SEQ ID NO:2099, SEQ ID NO:2100, SEQ ID NO:2101, SEQ ID NO:2102, SEQ ID NO:2103, SEQ ID NO:2104, or SEQ ID NO:2105.

Further, in a variety of illustrative embodiments, and particularly in those embodiments concerning methods and compositions relating to T cell non-Hodgkin's lymphoma, the polypeptides of the invention comprise at least a first isolated coding region that (a) comprises, (b) consists essentially of, or (c) consists of, the amino acid sequence of SEQ ID NO:2106, SEQ ID NO:2107, SEQ ID NO:2108, SEQ ID NO:2109, SEQ ID NO:2110, SEQ ID NO:2111, SEQ ID NO:2112, SEQ ID NO:2113, SEQ ID NO:2114, SEQ ID NO:2115, SEQ ID NO:2116, SEQ ID NO:2117, SEQ ID NO:2118, SEQ ID NO:2119, SEQ ID NO:2120, SEQ ID NO:2121, SEQ ID NO:2122, SEQ ID NO:2123, SEQ ID NO:2124, SEQ ID NO:2125, SEQ ID NO:2126, SEQ ID NO:2127, SEQ ID NO:2128, SEQ ID NO:2129, SEQ ID NO:2130, SEQ ID NO:2131, SEQ ID NO:2132, SEQ ID NO:2133, SEQ ID NO:2134, SEQ ID NO:2135, SEQ ID NO:2136, SEQ ID NO:2137, SEQ ID NO:2138, SEQ ID NO:2139, SEQ ID NO:2140, SEQ ID NO:2141, SEQ ID NO:2142, SEQ ID NO:2143, SEQ ID NO:2144, SEQ ID NO:2145, SEQ ID NO:2146, SEQ ID NO:2147, SEQ ID NO:2148, SEQ ID NO:2149, SEQ ID NO:2150, SEQ ID NO:2151, SEQ ID NO:2152, SEQ ID NO:2153, SEQ ID NO:2154, SEQ ID NO:2155, SEQ ID NO:2156, SEQ ID NO:2157, SEQ ID NO:2158, SEQ ID NO:2159, SEQ ID NO:2160, SEQ ID NO:2161, SEQ ID NO:2162, SEQ ID NO:2163, SEQ ID NO:2164, SEQ ID NO:2165, SEQ ID NO:2166, SEQ ID NO:2167, SEQ ID NO:2168, SEQ ID NO:2169, SEQ ID NO:2170, SEQ ID NO:2171, SEQ ID NO:2172, SEQ ID NO:2173, SEQ ID NO:2174, SEQ ID NO:2175, SEQ ID NO:2176, SEQ ID NO:2177, SEQ ID NO:2178, SEQ ID NO:2179, SEQ ID NO:2180, SEQ ID NO:2181, SEQ ID NO:2182, SEQ ID NO:2183, SEQ ID NO:2184, SEQ ID NO:2185, SEQ ID NO:2186, SEQ ID NO:2187, SEQ ID NO:2188, SEQ ID NO:2189; SEQ ID NO:2190, SEQ ID NO:2191, SEQ ID NO:2192, SEQ ID NO:2193, SEQ ID NO:2194, SEQ ID NO:2195, SEQ ID NO:2196, SEQ ID NO:2197, SEQ ID NO:2198, SEQ ID NO:2199, SEQ ID NO:2200, SEQ ID NO:2201, SEQ ID NO:2202, SEQ ID NO:2203, SEQ ID NO:2204, SEQ ID NO:2205, SEQ ID NO:2206, SEQ ID NO:2207, SEQ ID NO:2208, SEQ ID NO:2209, SEQ ID NO:2210, SEQ ID NO:2211, SEQ ID NO:2212, SEQ ID NO:2213, SEQ ID NO:2214, SEQ ID NO:2215, SEQ ID NO:2216, SEQ ID NO:2217, SEQ ID NO:2218, SEQ ID NO:2219, SEQ ID NO:2220, SEQ ID NO:2221, SEQ ID NO:2222, SEQ ID NO:2223, SEQ ID NO:2224, SEQ ID NO:2225, SEQ ID NO:2226, SEQ ID NO:2227, SEQ ID NO:2228, SEQ ID NO:2229, SEQ ID NO:2230, SEQ ID NO:2231, SEQ ID NO:2232, SEQ ID NO:2233, SEQ ID NO:2234, SEQ ID NO:2235, SEQ ID NO:2236, SEQ ID NO:2237, SEQ ID NO:2238, SEQ ID NO:2239, SEQ ID NO:2240, SEQ ID NO:2241, SEQ ID NO:2242, SEQ ID NO:2243, SEQ ID NO:2244, SEQ ID NO:2245, SEQ ID NO:2246, SEQ ID NO:2247, SEQ ID NO:2248, SEQ ID NO:2249, SEQ ID NO:2250, SEQ ID NO:2251, SEQ ID NO:2252, SEQ ID NO:2253, SEQ ID NO:2254, SEQ ID NO:2255, SEQ ID NO:2256, SEQ ID NO:2257, SEQ ID NO:2258, SEQ ID NO:2259, SEQ ID NO:2260, SEQ ID NO:2261, SEQ ID NO:2262, SEQ ID NO:2263, SEQ ID NO:2264, SEQ ID NO:2265, SEQ ID NO:2266, SEQ ID NO:2267, SEQ ID NO:2268, SEQ ID NO:2269, SEQ ID NO:2270, SEQ ID NO:2271, SEQ ID NO:2272, SEQ ID NO:2273, SEQ ID NO:2274, SEQ ID NO:2275, SEQ ID NO:2276, SEQ ID NO:2277, SEQ ID NO:2278, SEQ ID NO:2279, SEQ ID NO:2280, SEQ ID NO:2281, SEQ ID NO:2282, SEQ ID NO:2283, SEQ ID NO:2284, SEQ ID NO:2285, SEQ ID NO:2286, SEQ ID NO:2287, SEQ ID NO:2288, SEQ ID NO:2289, SEQ ID NO:2290, SEQ ID NO:2291, SEQ ID NO:2292, SEQ ID NO:2293, SEQ ID NO:2294, SEQ ID NO:2295, SEQ ID NO:2296, SEQ ID NO:2297, SEQ ID NO:2298, SEQ ID NO:2299, SEQ ID NO:2300, SEQ ID NO:2301, SEQ ID NO:2302, SEQ ID NO:2303, SEQ ID NO:2304, SEQ ID NO:2305, SEQ ID NO:2306, SEQ ID NO:2307, SEQ ID NO:2308, SEQ ID NO:2309, SEQ ID NO:2310, SEQ ID NO:2311, SEQ ID NO:2312, SEQ ID NO:2313, SEQ ID NO:2314, SEQ ID NO:2315, SEQ ID NO:2316 , SEQ ID NO:2317 , SEQ ID NO:2318 , SEQ ID NO:2319 , SEQ ID NO:2320, SEQ ID NO:2321, SEQ ID NO:2322, SEQ ID NO:2323, SEQ ID NO:2324, SEQ ID NO:2325, SEQ ID NO:2326, SEQ ID NO:2327, SEQ ID NO:2328, SEQ ID NO:2329, SEQ ID NO:2330, SEQ ID NO:2331, SEQ ID NO:2332, SEQ ID NO:2333, SEQ ID NO:2334, SEQ ID NO:2335, SEQ ID NO:2336, SEQ ID NO:2337, SEQ ID NO:2338, SEQ ID NO:2339, SEQ ID NO:2340, SEQ ID NO:2341, SEQ ID NO:2342, SEQ ID NO:2343, SEQ ID NO:2344, SEQ ID NO:2345, SEQ ID NO:2346, SEQ ID NO:2347, SEQ ID NO:2348, SEQ ID NO:2349, SEQ ID NO:2350, SEQ ID NO:2351, SEQ ID NO:2352, SEQ ID NO:2353, SEQ ID NO:2354, SEQ ID NO:2355, SEQ ID NO:2356, SEQ ID NO:2357, SEQ ID NO:2358, SEQ ID NO:2359, SEQ ID NO:2360, SEQ ID NO:2361, SEQ ID NO:2362, SEQ ID NO:2363, SEQ ID NO:2364, SEQ ID NO:2365, SEQ ID NO:2366, SEQ ID NO:2367, SEQ ID NO:2368, SEQ ID NO:2369, SEQ ID NO:2370, SEQ ID NO:2371, SEQ ID NO:2372, SEQ ID NO:2373, SEQ ID NO:2374, or SEQ ID NO:2375.

In additional illustrative embodiments, and particularly in those embodiments concerning methods and compositions relating to lymphoma, the polypeptides of the invention comprise at least a first isolated coding region that (a) comprises, (b) consists essentially of, or (c) consists of, the amino acid sequence of SEQ ID NO:2376, SEQ ID NO:2377, SEQ ID NO:2378, SEQ ID NO:2379, SEQ ID NO:2380, SEQ ID NO:2381, SEQ ID NO:2382, SEQ ID NO:2383, SEQ ID NO:2384, SEQ ID NO:2385, SEQ ID NO:2386, SEQ ID NO:2387, SEQ ID NO:2388, SEQ ID NO:2389, SEQ ID NO:2390, SEQ ID NO:2391, SEQ ID NO:2392, SEQ ID NO:2393, SEQ ID NO:2394, SEQ ID NO:2395, SEQ ID NO:2396, SEQ ID NO:2397, SEQ ID NO:2398, SEQ ID NO:2399, SEQ ID NO:2400, SEQ ID NO:2401, SEQ ID NO:2402, SEQ ID NO:2403, SEQ ID NO:2404, SEQ ID NO:2405, SEQ ID NO:2406, SEQ ID NO:2407, SEQ ID NO:2408, SEQ ID NO:2409, SEQ ID NO:2410, SEQ ID NO:2411, SEQ ID NO:2412, SEQ ID NO:2413, SEQ ID NO:2414, SEQ ID NO:2415, SEQ ID NO:2416, SEQ ID NO:2417, SEQ ID NO:2418, SEQ ID NO:2419, SEQ ID NO:2420, SEQ ID NO:2421, SEQ ID NO:2422, SEQ ID NO:2423, SEQ ID NO:2424, SEQ ID NO:2425, SEQ ID NO:2426, SEQ ID NO:2427, SEQ ID NO:2428, SEQ ID NO:2429, SEQ ID NO:2430, SEQ ID NO:2431, SEQ ID NO:2432, SEQ ID NO:2433, SEQ ID NO:2434, SEQ ID NO:2435, SEQ ID NO:2436, SEQ ID NO:2437, SEQ ID NO:2438, SEQ ID NO:2439, SEQ ID NO:2440, SEQ ID NO:2441, SEQ ID NO:2442, SEQ ID NO:2443, SEQ ID NO:2444, SEQ ID NO:2445, SEQ ID NO:2446, SEQ ID NO:2447, SEQ ID NO:2448, SEQ ID NO:2449, SEQ ID NO:2450, SEQ ID NO:2451, SEQ ID NO:2452, SEQ ID NO:2453, SEQ ID NO:2454, SEQ ID NO:2455, SEQ ID NO:2456, SEQ ID NO:2457, SEQ ID NO:2458, SEQ ID NO:2459, SEQ ID NO:2460, SEQ ID NO:2461, SEQ ID NO:2462, SEQ ID NO:2463, SEQ ID NO:2464, SEQ ID NO:2465, SEQ ID NO:2466, SEQ ID NO:2467, SEQ ID NO:2468, SEQ ID NO:2469, SEQ ID NO:2470, SEQ ID NO:2471, SEQ ID NO:2472, SEQ ID NO:2473, SEQ ID NO:2474, SEQ ID NO:2475, SEQ ID NO:2476, SEQ ID NO:2477, SEQ ID NO:2478, SEQ ID NO:2479, SEQ ID NO:2480, SEQ ID NO:2481, SEQ ID NO:2482, SEQ ID NO:2483, SEQ ID NO:2484, SEQ ID NO:2485, SEQ ID NO:2486, SEQ ID NO:2487, SEQ ID NO:2488, SEQ ID NO:2489, SEQ ID NO:2490, SEQ ID NO:2491, SEQ ID NO:2492, SEQ ID NO:2493, SEQ ID NO:2494, SEQ ID NO:2495, SEQ ID NO:2496, SEQ ID NO:2497, SEQ ID NO:2498, SEQ ID NO:2499, SEQ ID NO:2500, SEQ ID NO:2501, SEQ ID NO:2502, SEQ ID NO:2503, SEQ ID NO:2504, SEQ ID NO:2505, SEQ ID NO:2506, SEQ ID NO:2507, SEQ ID NO:2508, SEQ ID NO:2509, SEQ ID NO:2510, SEQ ID NO:2511, SEQ ID NO:2512, SEQ ID NO:2513, SEQ ID NO:2514, SEQ ID NO:2515, SEQ ID NO:2516, SEQ ID NO:2517, SEQ ID NO:2518, SEQ ID NO:2519, SEQ ID NO:2520, SEQ ID NO:2521, SEQ ID NO:2522, SEQ ID NO:2523, SEQ ID NO:2524, SEQ ID NO:2525, SEQ ID NO:2526, SEQ ID NO:2527, SEQ ID NO:2528, SEQ ID NO:2529, SEQ ID NO:2530, SEQ ID NO:2531, or SEQ ID NO:2532.

The polypeptides and proteins of the invention preferably comprise at least a first isolated coding region comprising an amino acid sequence that is encoded by at least a first nucleic acid segment that comprises an at least 21, 22, 23, 24, 25, 26, 27, 28, 29, or 30 contiguous nucleotide sequence of any one of SEQ ID NO:1 to SEQ ID NO:668.

The polypeptides and proteins of the invention may also preferably comprise one or more coding regions that comprise an amino acid sequence encoded by at least a first nucleic acid segment that comprises an at least about 31, 32, 33, 34, 35, 36, 37, 38, 39, or 40 contiguous nucleotide sequence of any one of SEQ ID NO:1 to SEQ ID NO:668. The polypeptides and proteins of the invention may also preferably comprise one or more coding regions that comprise an amino acid sequence encoded by at least a first nucleic acid segment that comprises an at least about 41, 42, 43, 44, 45, 46, 47, 48, 49, or 50 contiguous nucleotide sequence of any one of SEQ ID NO:1 to SEQ ID NO:668. The polypeptides and proteins of the invention may also preferably comprise one or more coding regions that comprise an amino acid sequence encoded by at least a first nucleic acid segment that comprises an at least about 51, 52, 53, 54, 55, 56, 57, 58, 59, or 60 contiguous nucleotide sequence of any one of SEQ ID NO:1 to SEQ ID NO:668. The polypeptides and proteins of the invention may also preferably comprise one or more coding regions that comprise an amino acid sequence encoded by at least a first nucleic acid segment that comprises an at least about 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 contiguous nucleotide sequence of any one of SEQ ID NO:1 to SEQ ID NO:668.

Likewise, the polypeptides and proteins of the invention may also preferably comprise one or more coding regions that comprise an amino acid sequence encoded by at least a first nucleic acid segment that comprises an at least about 110, 120, 130, 140, 150, 160, 170, 180, 190, 200, 220, 240, 260, 280, 300, 320, 340, 360, 380, 400, 420, 440, 460, 480, or 500 contiguous nucleotide sequence, even up to and including the entire sequence or the substantially entire sequence of any one of SEQ ID NO:1 to SEQ ID NO:668.

In a second important embodiment, there is provided a composition comprising at least a first isolated polynucleotide that comprises a nucleic acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:1 to SEQ ID NO:668. Exemplary preferred sequences are those that comprise a nucleic acid sequence that is at least about 85%, about 86%, about 87%, about 88%, about 89%, about 90%, about 91%, about 92%, about 93%, or about 94% identical to the nucleic acid sequence of any one of SEQ ID NO:1 to SEQ ID NO:668, with those sequences that comprise at least a nucleic acid sequence that is at least about 95%, about 96%, about 97%, about 98%, or about 99% identical to the nucleic acid sequence of any one of SEQ ID NO:1 to SEQ ID NO:668 being examples of particularly preferred sequences in the practice of the present invention.

In embodiments that relate particularly to compositions and methods for the detection, diagnosis, prognosis, prophylaxis, treatment, and therapy of Hodgkin's lymphoma exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:1 to SEQ ID NO:278 and SEQ ID NO:667 to SEQ ID NO:668, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of any one of SEQ ID NO:1 to SEQ ID NO:278 and SEQ ID NO:667 to SEQ ID NO:668, and even those sequences that comprise at least a first isolated nucleic acid segment that comprises a sequence that is at least about 95%, about 96%, about 97%, about 98%, or about 99% identical to the nucleic acid sequence of any one of SEQ ID NO:1 to SEQ ID NO:278 and SEQ ID NO:667 to SEQ ID NO:668. Such polynucleotides will preferably comprise one or more isolated coding region, each of which may (a) comprise, (b) consist essentially of, or (c) consist of, the nucleic acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, or SEQ ID NO:278.

In embodiments that relate particularly to compositions and methods for the detection, diagnosis, prognosis, prophylaxis, treatment, and therapy of follicular lymphoma, exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:279 to SEQ ID NO:436, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of any one of SEQ ID NO:279 to SEQ ID NO:436, and even those sequences that comprise at least a first isolated nucleic acid segment that comprises a sequence that is at least about 95%, about 96%, about 97%, about 98%, or about 99% identical to the nucleic acid sequence of any one of SEQ ID NO:279 to SEQ ID NO:436. Such polynucleotides will preferably comprise one or more isolated coding region, each of which may (a) comprise, (b) consist essentially of, or (c) consist of, the nucleic acid sequence of SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:383, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, or SEQ ID NO:436.

In embodiments that relate particularly to compositions and methods for the detection, diagnosis, prognosis, prophylaxis, treatment, and therapy of B cell non-Hodgkin's lymphoma, exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:437 to SEQ ID NO:528 and SEQ ID NO:665 to SEQ ID NO:668, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of any one of SEQ ID NO:437 to SEQ ID NO:528 and SEQ ID NO:665 to SEQ ID NO:668, and even those sequences that comprise at least a first isolated nucleic acid segment that comprises a sequence that is at least about 95%, about 96%, about 97%, about 98%, or about 99% identical to the nucleic acid sequence of any one of SEQ ID NO:437 to SEQ ID NO:528 and SEQ ID NO:665 to SEQ ID NO:668. Such polynucleotides will preferably comprise one or more isolated coding region, each of which may (a) comprise, (b) consist essentially of, or (c) consist of, the nucleic acid sequence of SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:487, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:503, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, SEQ ID NO:524, SEQ ID NO:525, SEQ ID NO:526, SEQ ID NO:527, or SEQ ID NO:528.

In embodiments that relate particularly to compositions and methods for the detection, diagnosis, prognosis, prophylaxis, treatment, and therapy of T cell non-Hodgkin's lymphoma, exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:529 to SEQ ID NO:610 and SEQ ID NO:665 or SEQ ID NO:666, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of any one of SEQ ID NO:529 to SEQ ID NO:610 and SEQ ID NO:665 or SEQ ID NO:666, and even those sequences that comprise at least a first isolated nucleic acid segment that comprises a sequence that is at least about 95%, about 96%, about 97%, about 98%, or about 99% identical to the nucleic acid sequence of any one of SEQ ID NO:529 to SEQ ID NO:610 and SEQ ID NO:665 or SEQ ID NO:666. Such polynucleotides will preferably comprise one or more isolated coding region, each of which may (a) comprise, (b) consist essentially of, or (c) consist of, the nucleic acid sequence of SEQ ID NO:529, SEQ ID NO:530, SEQ ID NO:531, SEQ ID NO:532, SEQ ID NO:533, SEQ ID NO:534, SEQ ID NO:535, SEQ ID NO:536, SEQ ID NO:537, SEQ ID NO:538, SEQ ID NO:539, SEQ ID NO:540, SEQ ID NO:541, SEQ ID NO:542, SEQ ID NO:543, SEQ ID NO:544, SEQ ID NO:545, SEQ ID NO:546, SEQ ID NO:547, SEQ ID NO:548, SEQ ID NO:549, SEQ ID NO:550, SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, or SEQ ID NO:610.

In embodiments that relate particularly to compositions and methods for the detection, diagnosis, prognosis, prophylaxis, treatment, and therapy of lymphoma, exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of any one of SEQ ID NO:611 to SEQ ID NO:664, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of any one of SEQ ID NO:611 to SEQ ID NO:664, and even those sequences that comprise at least a first isolated nucleic acid segment that comprises a sequence that is at least about 95%, about 96%, about 97%, about 98%, or about 99% identical to the nucleic acid sequence of any one of SEQ ID NO:611 to SEQ ID NO:664. Such polynucleotides will preferably comprise one or more isolated coding region, each of which may (a) comprise, (b) consist essentially of, or (c) consist of, the nucleic acid sequence of SEQ ID NO:611, SEQ ID NO:612, SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:627, SEQ ID NO:628, SEQ ID NO:629, SEQ ID NO:630, SEQ ID NO:631, SEQ ID NO:632, SEQ ID NO:633, SEQ ID NO:634, SEQ ID NO:635, SEQ ID NO:636, SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, SEQ ID NO:662, SEQ ID NO:663, or SEQ ID NO:664.

In embodiments that relate particularly to compositions and methods for the detection, diagnosis, prognosis, prophylaxis, treatment, and therapy of chronic lymphocytic leukemia, exemplary preferred polynucleotide compositions include those compositions that comprise at least a first isolated nucleic acid segment that comprises a sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the nucleic acid sequence of SEQ ID NO:665 or SEQ ID NO:666, and those that comprise at least a first isolated nucleic acid segment that comprises a sequence that is at least about 85%, about 86%, about 87%, about 88%, about 89%, about 90%, about 91%, about 92%, about 93%, or about 94% identical to the nucleic acid sequence of SEQ ID NO:665 or SEQ ID NO:666, and even those sequences that comprise at least a first isolated nucleic acid segment that comprises a sequence that is at least about 95%, about 96%, about 97%, about 98%, or about 99% identical to the nucleic acid sequence of SEQ ID NO:665 or SEQ ID NO:666.

Exemplary polynucleotides of the present invention may be of any suitable length, depending upon the particular application thereof, and encompass those polynucleotides that (a) are at least about, or (b) comprise at least a first isolated nucleic acid segment that is at least about 30,40, 50,60, 70, 80, 90, 100, 110, 120, 120, 140, 150, 160, 170, 180, 190, 200, 210, 220, 230, 240, 250, 260, 270, 280, 290, 300, 320, 340, 360, 380, 400, 420, 440, 460, 480, 500, 520, 540, 560, 580, 600, 625, 650, 675, 700, 750, 800, 850, 900, 950, or 1000 or so nucleic acids in length, as well as longer polynucleotides that (a) are at least about, or (b) comprise at least a first isolated nucleic acid segment that is at least about 1000, 1025, 1050, 1075, 1100, 150, 1200, 1250, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, or 3000 or so nucleic acids in length, as well as substantially larger polynucleotides that (a) are at least about, or (b) comprise at least a first isolated nucleic acid segment that is at least about 3500, 4000, 4500, 5000, 5500, 6000, 6500, 7000, 7500, 8000, 8500, 9000, 9500 or 10,000 so nucleic acids in length. Of course, the polynucleotides and nucleic acid segments of the invention may also encompass any intermediate lengths or integers within the stated ranges.

The compositions of the present invention may comprise a single polypeptide or polynucleotide, or alternatively, may comprise two or more such hematological malignancy compounds, such as for example, two or more polypeptides, two or more polynucleotides, or even combinations of one or more peptides or polypeptides, along with one or more polynucleotides. When two or more polypeptides are contemplated for particular applications, the second and/or third and/or fourth, etc. isolated peptides and/or polypeptides will preferably comprise at least one isolated coding region that comprises an amino acid sequence that is at least about 91%, 93%, 95%, 97%, or 99% identical to the amino acid sequence of any one of SEQ ID NO:669 to SEQ ID NO:2532. Alternatively, the polynucleotides of the invention may comprise one or more coding regions that encode a first fusion protein or peptide, such as an adjuvant-coding region fused in correct reading frame to one or more of the disclosed hematological malignancy peptides or polypeptides. Alternatively, the fusion protein may comprise a hematological malignancy polypeptide or peptide fused, in correct reading frame, to a detectable protein or peptide, or to an immunostimulant protein or peptide, or other such construct. Fusion proteins such as these are particularly useful in those embodiments relating to diagnosis, detection, and therapy of one or more of the hematological malignancies as discussed herein.

The invention also provides a composition comprising at least a first hybridoma cell line that produces a monoclonal antibody having immunospecificity for one or more of the peptides or polypeptides as disclosed herein, or at least a first monoclonal antibody, or an antigen-binding fragment thereof, that has immunospecificity for such a peptide or polypeptide. The antigen binding fragments may comprise a light chain variable region, a heavy-chain variable region, a Fab fragment, a F(ab)₂ fragment, an Fv fragment, an scFv fragment, or an antigen-binding fragment of such an antibody.

The invention also provides a composition comprising at least a first isolated antigen-presenting cell that expresses a peptide or polypeptide as disclosed herein, or a plurality of isolated T cells that specifically react with such a peptide or polypeptide. Such pluralities of isolated T cells may be stimulated or expanded by contacting the T cells with one or more peptides or polypeptides as described herein. The T cells may be cloned prior to expansion, and may be obtained from bone marrow, a bone marrow fraction, peripheral blood, or a peripheral blood fraction from a healthy mammal, or from a mammal that is afflicted with at least a first hematological malignancy such as leukemia or lymphoma.

As descrbied above, the isolated coding regions within the polypeptides of the invention may be on the order of from 25 to about 1000 amino acids in length, or alternatively, may be on the order of from 50 to about 900 amino acids in length, from 75 to about 800 amino acids in length, from 100 to about 700 amino acids in length, or from 125 to about 600 amino acids in length, or any other such suitable range.

The isolated nucleic acid segments that encode such isolated coding regions may be on the order of from 50 to about 10,000 nucleotides in length, from 150 to about 8000 nucleotides in length, from 250 to about 6000 nucleotides in length, from 350 to about 4000 nucleotides in length, or from 450 to about 2000 nucleotides in length, or any other such suitable range.

The nucleic acid segment may be operably positioned under the control of at least a first heterologous, recombinant promoter, such as a tissue-specific, cell-specific, inducible, or otherwise regulated promoter. Such promoters may be further controlled or regulated by the presence of one or more additional enhancers or regulatory regions depending upon the particular cell type in which expression of the polynucleotide is desired. The polynucleotides and nucleic acid segments of the invention may also be comprised within a vector, such as a plasmid, or viral vector. The polypeptides and polynucleotides of the invention may also be comprised within a host cell, such as a recombinant host cell, or a human host cell such as a blood or bone marrow cell.

The polynucleotides of the invention may comprise at least a first isolated nucleic acid segment is operably attached, in frame, to at least a second isolated nucleic acid segment, such that the polynucleotide encodes a fusion protein in which the first peptide or polypeptide is linked to the second peptide or polypeptide.

The polypeptides of the present invention may comprise a contiguous amino acid coding region of any suitable length, such as for example, those of about 2000, about 1900, about 1850, about 1800, about 1750, about 1700, about 1650, about 1600, about 1550, about 1500, about 1450, about 1400, about 1350, about 1300, about 1250, about 1200, about 1150, about 1100 amino acids, or about 1000 or so amino acids in length. Likewise, the polypeptides and peptides of the present invention may comprise slightly shorter contiguous amino acid coding regions, such as for example, those of about 950, about 900, about 850, about 800, about 750, about 700, about 650, about 600, about 550, about 500, about 450, about 400, about 350, about 300, about 250, about 200, about 150, or even about 100 amino acids or so in length.

In similar fashion, the polypeptides and peptides of the present invention may comprise even smaller contiguous amino acid coding regions, such as for example, those of about 95, about 90, about 85, about 80, about 75, about 70, about 65, about 60, about 55, about 50, about 45, about 40, about 35, about 30, about 25, about 20, or even about 15 amino acids or so in length.

In all such embodiments, those peptides and polypeptides having intermediate lengths including all integers within the preferred ranges (e.g., those peptides and polypeptides that comprise at least a first coding region of at least about 94, about 93, about 92, about 91, about 89, about 88, about 87, about 86, about 84, about 83, about 82, about 81, about 79, about 78, about 77, about 76, about 74, about 73, about 72, about 71, about 69, about 68, about 67, about 66 or so amino acids in length, etc.) are all contemplated to fall within the scope of the present invention.

In particular embodiments, the peptides and polypeptides of the present invention may comprise at least a first coding region that comprises a sequence of at least about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 13, or about 34, or about 35, or about 36, or about 37, or about 38, or about 39, or about 40, or about 41, or about 42, or about 43, or about 44, or about 45, or about 46, or about 47, or about 48, or about 49, or about 50 contiguous amino acids as disclosed in any one or more of SEQ ID NO:669 through SEQ ID NO:2532 herein.

In other embodiments, the peptides and polypeptides of the present invention may comprise at least a first coding region that comprises a sequence of at least about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, about 91, or about 92, or about 93, or about 94, or about 95, or about 96, or about 97, or about 98, or about 99, or 100 contiguous amino acids as disclosed in any one or more of SEQ ID NO:669 through SEQ ID NO:2532 herein.

In still other embodiments, the preferred peptides and polypeptides of the present invention comprise at least a first coding region that comprises a sequence of at least about 100, 125, 150, 175, 200, 225, 250, 275, 300, 325, 350, 375, 400, 425, 450, 475, 500, 525, 550, 575, 600, 625, 650, 675 or 700 or more contiguous amino acids as disclosed in any one or more of SEQ ID NO:669 through SEQ ID NO:2532 herein. The preferred peptides and polypeptides of the present invention may also comprise at least a first coding region that comprises a sequence of at least about 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375 or 1400 or more contiguous amino acids as disclosed in any one or more of SEQ ID NO:669 through SEQ ID NO:2532 herein. Likewise, in still other embodiments, the preferred peptides and polypeptides of the present invention comprise at least a first coding region that comprises a sequence of at least about 1500, 1525, 1550, 1575, 1600, 1625, 1650, 1675, 1700, 1725, 1750, 1775, 1800, 1825, 1850, 1875, 1900, 1925, 1950, 1975, or 2000 or more contiguous amino acids as disclosed in any one or more of SEQ ID NO:669 through SEQ ID NO:2532 herein.

The polypeptides of the invention typically will comprise at least a first contiguous amino acid sequence according to any one of SEQ ID NO:669 through SEQ ID NO:2532, but may also, optionally comprise at least a second, at least a third, or even at least a fourth or greater contiguous amino acid sequence according to any one of SEQ ID NO:669 through SEQ ID NO:2532. A single polypeptide may contain only a single coding region, or alternatively, a single polypeptide may comprise a plurality of identical or distinctly different contiguous amino acid sequences in accordance with any one of SEQ ID NO:669 through SEQ ID NO:2532. In fact, the polypeptide may comprise a plurality of the same contiguous amino acid sequences, or they may comprise one or more different contiguous amino acid sequences disclosed in SEQ ID NO:669 through SEQ ID NO:2532. For example, a single polypeptide can comprise a single contiguous amino acid sequence from one or more of SEQ ID NO:669 through SEQ ID NO:2532, or alternatively, may comprise two or more distinctly different contiguous amino acid sequences from one or more of SEQ ID NO:669 through SEQ ID NO:2532. In fact, the polypeptide may comprise 2, 3, 4, or even 5 distinct contiguous amino sequences as disclosed in any of SEQ ID NO:669 through SEQ ID NO:2532. Alternatively, a single polypeptide may comprise 2, 3, 4, or even 5 distinct coding regions. For example, a polypeptide may comprise at least a first coding region that comprises a first contiguous amino acid sequence as disclosed in any of SEQ ID NO:669 through SEQ ID NO:2532, and at least a second coding region that comprises a second contiguous amino acid sequence as disclosed in any of SEQ ID NO:669 through SEQ ID NO:2532. In contrast, a polypeptide may comprise at least a first coding region that comprises a first contiguous amino acid sequence as disclosed in any of SEQ ID NO:669 through SEQ ID NO:2532, and at least a second coding region that comprises a second distinctly different peptide or polypeptide, such as for example, an adjuvant or an immunostimulant peptide or polypeptide.

In such cases, the two coding regions may be separate on the same polypeptide, or the two coding regions may be operatively attached, each in the correct reading frame, such that a fusion polypeptide is produced, in which the first amino acid sequence of the first coding region is linked to the second amino acid sequence of the second coding region.

Throughout this disclosure, a phrase such as “a sequence as disclosed in SEQ ID NO:1 to SEQ ID NO:4” is intended to encompass any and all contiguous sequences disclosed by any one of these sequence identifiers. That is to say, “a sequence as disclosed in any of SEQ ID NO:1 through SEQ ID NO:4” means any sequence that is disclosed in any one of SEQ ID NO:1, SEQ ID NO:2, SEQ ID NO:3, or SEQ ID NO:4. Likewise, “a sequence as disclosed in any of SEQ ID NOs:25 to 37” means any sequence that is disclosed in any one of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, and so forth.

Likewise, “at least a first sequence from any one of SEQ ID NO:55 to SEQ ID NO:62” is intended to refer to a first sequence that is disclosed in any one of SEQ ID NO:55, SEQ ID NO:56, SEQ ID NO:57, SEQ ID NO:58, SEQ ID NO:59, SEQ ID NO:60, SEQ ID NO:61, or SEQ ID NO:62.

It will also be understood that the kits, and compositions of the present invention comprise in an overall and general sense at least one or more particular polynucleotides, polypeptides, and peptides that comprise one or more contiguous sequence regions from one or more of the nucleic acid sequences disclosed herein in SEQ ID NO:1 through SEQ ID NO:668 or from one or more of the amino acid sequences disclosed herein in SEQ ID NO:669 through SEQ ID NO:2532, and that such peptide, polypeptide and polynucleotide compositions may be used in one or more of the particular methods and uses disclosed herein for the diagnosis, detection, prophylaxis, and therapy of one or more hematological cancers, and in particular, lymphomas of a variety of specific types. It will also be understood to the skilled artisan having benefit of the teachings of the present Specification, that the peptide and polypeptide compositions may be used to generate a T cell or an immune response in an animal, and that such compositions may also be administered to an animal from which immunospecific antibodies and antigen binding fragments may be isolated or identified that specifically bind to such peptides or polypeptides. Such an artisan will also recognize that the polynucleotides identified by the present disclosure may be used to produce such peptides, polypeptides, antibodies, and antigen binding fragments, by recombinant protein production methodologies that are also within the capability of the skilled artisan having benefit of the specific amino acid and nucleic acid sequences provided herein.

Likewise, it will be understood by a skilled artisan in the field, that one or more of the disclosed compositions may used in one or more diagnostic or detection methodologies to identify certain antibodies, peptides, polynucleotides, or polypeptides in a biological sample, in a host cell, or even within the body or tissues of an animal. It will be understood by a skilled artisan in the field, that one or more of the disclosed nucleic acid or amino acid compositions may used in the preparation or manufacture of one or more medicaments for use in the diagnosis, detection, prognosis, prophylaxis, or therapy of one or more hematological malignancies in an animal, and particularly those malignant conditions disclosed and claimed herein.

It will also be readily apparent to those of skill in the art, that the methods, kits, and uses, of the present invention preferably employ one or more of the compounds and/or compositions disclosed herein that comprise one or more contiguous nucleotide sequences as may be presented in SEQ ID NO:1 through SEQ ID NO:10 of the attached sequence listing, as well as those compounds and compositions that comprise one or more contiguous nucleotide sequences as may be presented in SEQ ID NO:21 through SEQ ID NO:30, SEQ ID NO:31 through SEQ ID NO:40, SEQ ID NO:41 through SEQ ID NO:50, SEQ ID NO:51 through SEQ ID NO:60, SEQ ID NO:61 through SEQ ID NO:70, SEQ ID NO:71 through SEQ ID NO:80, SEQ ID NO:81 through SEQ ID NO:90, SEQ ID NO:91 through SEQ ID NO:100, SEQ ID NO:101 through SEQ ID NO:110, SEQ ID NO:111 through SEQ ID NO:120, SEQ ID NO:121 through SEQ ID NO:130, SEQ ID NO:131 through SEQ ID NO:140, SEQ ID NO:141 through SEQ ID NO:150, SEQ ID NO:151 through SEQ ID NO:160, SEQ ID NO:161 through SEQ ID NO:170, SEQ ID NO:171 through SEQ ID NO:180, SEQ ID NO:181 through SEQ ID NO:190, SEQ ID NO:191 through SEQ ID NO:200, SEQ ID NO:201 through SEQ ID NO:210, SEQ ID NO:211 through SEQ ID NO:220, SEQ ID NO:221 through SEQ ID NO:230, SEQ ID NO:231 through SEQ ID NO:240, SEQ ID NO:241 through SEQ ID NO:250, SEQ ID NO:251 through SEQ ID NO:260, SEQ ID NO:261 through SEQ ID NO:270, SEQ ID NO:271 through SEQ ID NO:280, SEQ ID NO:281 through SEQ ID NO:290, SEQ ID NO:291 through SEQ ID NO:300, SEQ ID NO:301 through SEQ ID NO:310, SEQ ID NO:311 through SEQ ID NO:320, SEQ ID NO:321 through SEQ ID NO:330, SEQ ID NO:331 through SEQ ID NO:340, SEQ ID NO:341 through SEQ ID NO:350, SEQ ID NO:351 through SEQ ID NO:360, SEQ ID NO:361 through SEQ ID NO:370, SEQ ID NO:371 through SEQ ID NO:380, SEQ ID NO:381 through SEQ ID NO:390, SEQ ID NO:391 through SEQ ID NO:400, SEQ ID NO:401 through SEQ ID NO:410, SEQ ID NO:411 through SEQ ID NO:420, SEQ ID NO:421 through SEQ ID NO:430, SEQ ID NO:431 through SEQ ID NO:440, SEQ ID NO:441 through SEQ ID NO:450, SEQ ID NO:451 through SEQ ID NO:460, SEQ ID NO:461 through SEQ ID NO:470, SEQ ID NO:471 through SEQ ID NO:480, SEQ ID NO:481 through SEQ ID NO:490, SEQ ID NO:491 through SEQ ID NO:500, SEQ ID NO:501 through SEQ ID NO:510, SEQ ID NO:511 through SEQ ID NO:520, SEQ ID NO:521 through SEQ ID NO:530, SEQ ID NO:531 through SEQ ID NO:540, SEQ ID NO:541 through SEQ ID NO:550, SEQ ID NO:551 through SEQ ID NO:560, SEQ ID NO:561 through SEQ ID NO:570, SEQ ID NO:571 through SEQ ID NO:580, SEQ ID NO:581 through SEQ ID NO:590, SEQ ID NO:591 through SEQ ID NO:600, SEQ ID NO:601 through SEQ ID NO:610, SEQ ID NO:611 through SEQ ID NO:620, SEQ ID NO:621 through SEQ ID NO:630, SEQ ID NO:631 through SEQ ID NO:640, SEQ ID NO:641 through SEQ ID NO:650, SEQ ID NO:651 through SEQ ID NO:660, and SEQ ID NO:661 through SEQ ID NO:669.

Likewise, it will also be readily apparent to those of skill in the art, that the methods, kits, and uses, of the present invention may also employ one or more of the compounds and compositions disclosed herein that comprise one or more contiguous amino acid sequences as may be presented in SEQ ID NO:669 through SEQ ID NO:678 of the attached sequence listing, as well as those compounds and compositions that comprise one or more contiguous amino acid sequences as may be presented in SEQ ID NO:679 through SEQ ID NO:688, SEQ ID NO:689 through SEQ ID NO:698, SEQ ID NO:699 through SEQ ID NO:708, SEQ ID NO:709 through SEQ ID NO:718, SEQ ID NO:719 through SEQ ID NO:728, SEQ ID NO:729 through SEQ ID NO:738, SEQ ID NO:739 through SEQ ID NO:748, SEQ ID NO:749 through SEQ ID NO:758, SEQ ID NO:759 through SEQ ID NO:768, SEQ ID NO:769 through SEQ ID NO:778, SEQ ID NO:779 through SEQ ID NO:788, SEQ ID NO:789 through SEQ ID NO:798, SEQ ID NO:799 through SEQ ID NO:808, SEQ ID NO:809 through SEQ ID NO:818, SEQ ID NO:819 through SEQ ID NO:828, SEQ ID NO:829 through SEQ ID NO:838, SEQ ID NO:839 through SEQ ID NO:848, SEQ ID NO:849 through SEQ ID NO:858, SEQ ID NO:859 through SEQ ID NO:868, SEQ ID NO:869 through SEQ ID NO:878, SEQ ID NO:879 through SEQ ID NO:888, SEQ ID NO:889 through SEQ ID NO:898, SEQ ID NO:899 through SEQ ID NO:908, SEQ ID NO:909 through SEQ ID NO:918, SEQ ID NO:919 through SEQ ID NO:928, SEQ ID NO:929 through SEQ ID NO:938, SEQ ID NO:939 through SEQ ID NO:948, SEQ ID NO:949 through SEQ ID NO:958, SEQ ID NO:959 through SEQ ID NO:968, SEQ ID NO:969 through SEQ ID NO:978, SEQ ID NO:979 through SEQ ID NO:988, SEQ ID NO:989 through SEQ ID NO:998, SEQ ID NO:999 through SEQ ID NO:1008, SEQ ID NO:1009 through SEQ ID NO:1018, SEQ ID NO:1019 through SEQ ID NO:1028, SEQ ID NO:1029 through SEQ ID NO:1038, SEQ ID NO:1039 through SEQ ID NO:1048, SEQ ID NO:1049 through SEQ ID NO:1058, SEQ ID NO:1059 through SEQ ID NO:1068, SEQ ID NO:1069 through SEQ ID NO:1078, SEQ ID NO:1079 through SEQ ID NO:1088, SEQ ID NO:1089 through SEQ ID NO:1098, SEQ ID NO:1099 through SEQ ID NO:1108, SEQ ID NO:1109 through SEQ ID NO:1118, SEQ ID NO:1119 through SEQ ID NO:1128, SEQ ID NO:1129 through SEQ ID NO:1138, SEQ ID NO:1139 through SEQ ID NO:1148, SEQ ID NO:1149 through SEQ ID NO:1158, SEQ ID NO:1159 through SEQ ID NO:1168, SEQ ID NO:1169 through SEQ ID NO:1178, SEQ ID NO:1179 through SEQ ID NO:1188, SEQ ID NO:1189 through SEQ ID NO:1198, SEQ ID NO:1199 through SEQ ID NO:1208, SEQ ID NO:1209 through SEQ ID NO:1218, SEQ ID NO:1219 through SEQ ID NO:1228, SEQ ID NO:1229 through SEQ ID NO:1238, SEQ ID NO:1239 through SEQ ID NO:1248, SEQ ID NO:1249 through SEQ ID NO:1258, SEQ ID NO:1259 through SEQ ID NO:1268, SEQ ID NO:1269 through SEQ ID NO:1278, SEQ ID NO:1279 through SEQ ID NO:1288, SEQ ID NO:1289 through SEQ ID NO:1298, SEQ ID NO:1299 through SEQ ID NO:1308, SEQ ID NO:1309 through SEQ ID NO:1318, SEQ ID NO:1319 through SEQ ID NO:1328, SEQ ID NO:1329 through SEQ ID NO:1338, SEQ ID NO:1339 through SEQ ID NO:1348, SEQ ID NO:1349 through SEQ ID NO:1358, SEQ ID NO:1359 through SEQ ID NO:1368, SEQ ID NO:1369 through SEQ ID NO:1378, SEQ ID NO:1379 through SEQ ID NO:1388, SEQ ID NO:1389 through SEQ ID NO:1398, SEQ ID NO:1399 through SEQ ID NO:1408, SEQ ID NO:1409 through SEQ ID NO:1418, SEQ ID NO:1419 through SEQ ID NO:1428, SEQ ID NO:1429 through SEQ ID NO:1438, SEQ ID NO:1439 through SEQ ID NO:1448, SEQ ID NO:1449 through SEQ ID NO:1458, SEQ ID NO:1459 through SEQ ID NO:1968, SEQ ID NO:1469 through SEQ ID NO:1478, SEQ ID NO:1479 through SEQ ID NO:1488, SEQ ID NO:1489 through SEQ ID NO:1498, SEQ ID NO:1499 through SEQ ID NO:1508, SEQ ID NO:1509 through SEQ ID NO:1518, SEQ ID NO:1519 through SEQ ID NO:1528, SEQ ID NO:1529 through SEQ ID NO:1538, SEQ ID NO:1539 through SEQ ID NO:1548, SEQ ID NO:1549 through SEQ ID NO:1558, SEQ ID NO:1559 through SEQ ID NO:1568, SEQ ID NO:1569 through SEQ ID NO:1578, SEQ ID NO:1579 through SEQ ID NO:1588, SEQ ID NO:1589 through SEQ ID NO:1598, SEQ ID NO:1599 through SEQ ID NO:1608, SEQ ID NO:1609 through SEQ ID NO:1618, SEQ ID NO:1619 through SEQ ID NO:1628, SEQ ID NO:1629 through SEQ ID NO:1638, SEQ ID NO:1639 through SEQ ID NO:1648, SEQ ID NO:1649 through SEQ ID NO:1658, SEQ ID NO:1659 through SEQ ID NO:1668, SEQ ID NO:1669 through SEQ ID NO:1678, SEQ ID NO:1679 through SEQ ID NO:1688, SEQ ID NO:1689 through SEQ ID NO:1698, SEQ ID NO:1699 through SEQ ID NO:1708, SEQ ID NO:1709 through SEQ ID NO:1718, SEQ ID NO:1719 through SEQ ID NO:1728, SEQ ID NO:1729 through SEQ ID NO:1738, SEQ ID NO:1739 through SEQ ID NO:1748, SEQ ID NO:1749 through SEQ ID NO:1758, SEQ ID NO:1759 through SEQ ID NO:1768, SEQ ID NO:1769 through SEQ ID NO:1778, SEQ ID NO:1779 through SEQ ID NO:1788, SEQ ID NO:1789 through SEQ ID NO:1798, SEQ ID NO:1799 through SEQ ID NO:1808, SEQ ID NO:1809 through SEQ ID NO:1818, SEQ ID NO:1819 through SEQ ID NO:1828, SEQ ID NO:1829 through SEQ ID NO:1838, SEQ ID NO:1839 through SEQ ID NO:1848, SEQ ID NO:1849 through SEQ ID NO:1858, SEQ ID NO:1859 through SEQ ID NO:1868, SEQ ID NO:1869 through SEQ ID NO:1878, SEQ ID NO:1879 through SEQ ID NO:1888, SEQ ID NO:1889 through SEQ ID NO:1898, SEQ ID NO:1899 through SEQ ID NO:1908, SEQ ID NO:1909 through SEQ ID NO:1918, SEQ ID NO:1919 through SEQ ID NO:1928, SEQ ID NO:1929 through SEQ ID NO:1938, SEQ ID NO:1939 through SEQ ID NO:1948, SEQ ID NO:1949 through SEQ ID NO:1958, SEQ ID NO:1959 through SEQ ID NO:1968, SEQ ID NO:1969 through SEQ ID NO:1978, SEQ ID NO:1979 through SEQ ID NO:1988, SEQ ID NO:1989 through SEQ ID NO:1998, SEQ ID NO:1999 through SEQ ID NO:2008, SEQ ID NO:2009 through SEQ ID NO:2018, SEQ ID NO:2019 through SEQ ID NO:2028, SEQ ID NO:2029 through SEQ ID NO:2038, SEQ ID NO:2039 through SEQ ID NO:2048, SEQ ID NO:2049 through SEQ ID NO:2058, SEQ ID NO:2059 through SEQ ID NO:2068, SEQ ID NO:2069 through SEQ ID NO:2078, SEQ ID NO:2079 through SEQ ID NO:2088, SEQ ID NO:2089 through SEQ ID NO:2098, SEQ ID NO:2099 through SEQ ID NO:2108, SEQ ID NO:2109 through SEQ ID NO:2118, SEQ ID NO:2119 through SEQ ID NO:2128, SEQ ID NO:2129 through SEQ ID NO:2138, SEQ ID NO:2139 through SEQ ID NO:2148, SEQ ID NO:2149 through SEQ ID NO:2158, SEQ ID NO:2159 through SEQ ID NO:2168, SEQ ID NO:2169 through SEQ ID NO:2178, SEQ ID NO:2179 through SEQ ID NO:2188, SEQ ID NO:2189 through SEQ ID NO:2198, SEQ ID NO:2199 through SEQ ID NO:2208, SEQ ID NO:2209 through SEQ ID NO:2218, SEQ ID NO:2219 through SEQ ID NO:2228, SEQ ID NO:2229 through SEQ ID NO:2238, SEQ ID NO:2239 through SEQ ID NO:2248, SEQ ID NO:2249 through SEQ ID NO:2258, SEQ ID NO:2259 through SEQ ID NO:2268, SEQ ID NO:2269 through SEQ ID NO:2278, SEQ ID NO:2279 through SEQ ID NO:2288, SEQ ID NO:2289 through SEQ ID NO:2298, SEQ ID NO:2299 through SEQ ID NO:2308, SEQ ID NO:2309 through SEQ ID NO:2318, SEQ ID NO:2319 through SEQ ID NO:2328, SEQ ID NO:2329 through SEQ ID NO:2338, SEQ ID NO:2339 through SEQ ID NO:2348, SEQ ID NO:2349 through SEQ ID NO:2358, SEQ ID NO:2359 through SEQ ID NO:2368, SEQ ID NO:2369 through SEQ ID NO:2378, SEQ ID NO:2379 through SEQ ID NO:2388, SEQ ID NO:2389 through SEQ ID NO:2398, SEQ ID NO:2399 through SEQ ID NO:2408, SEQ ID NO:2409 through SEQ ID NO:2418, SEQ ID NO:2419 through SEQ ID NO:2428, SEQ ID NO:2429 through SEQ ID NO:2438, SEQ ID NO:2439 through SEQ ID NO:2448, SEQ ID NO:2449 through SEQ ID NO:2458, SEQ ID NO:2459 through SEQ ID NO:2468, SEQ ID NO:2469 through SEQ ID NO:2478, SEQ ID NO:2479 through SEQ ID NO:2488, SEQ ID NO:2489 through SEQ ID NO:2498, SEQ ID NO:2499 through SEQ ID NO:2508, SEQ ID NO:2509 through SEQ ID NO:2518, SEQ ID NO:2519 through SEQ ID NO:2532 of the attached sequence listing.

### BRIEF DESCRIPTION OF THE DRAWINGS AND THE APPENDICES

The invention may be understood by reference to the following description taken in conjunction with the accompanying drawings, in which like reference numerals identify like elements, and in which:

FIG. 1 illustrates a schematic outline of the microarray chip technology approach used to identify the cDNA targets of the present invention as described Section 5.1;

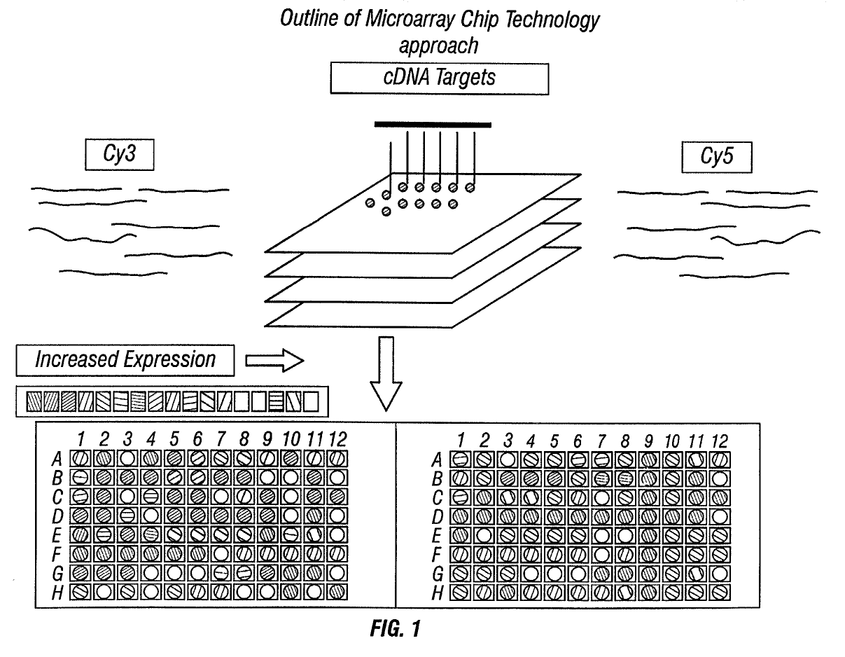

FIG. 2 illustrates a schematic outline of the general protcol for in vitro whole gene CD8 T cell priming procedure used to generate antigen-specific lines and to identify clones of interest;

FIG. 3 illustrates a schematic outline of the general protcol for in vitro whole gene CD4 T cell priming procedure used to generate antigen-specific lines and to identify clones of interest;

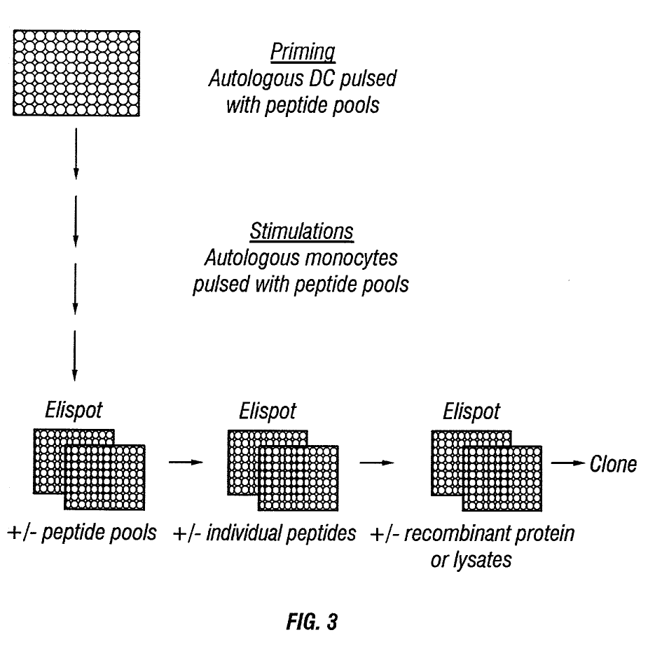

FIG. 4 illustrates the results of Coronin 1A mRNA expression in lymphoma patients and normal tissues as determined by real-time PCR.

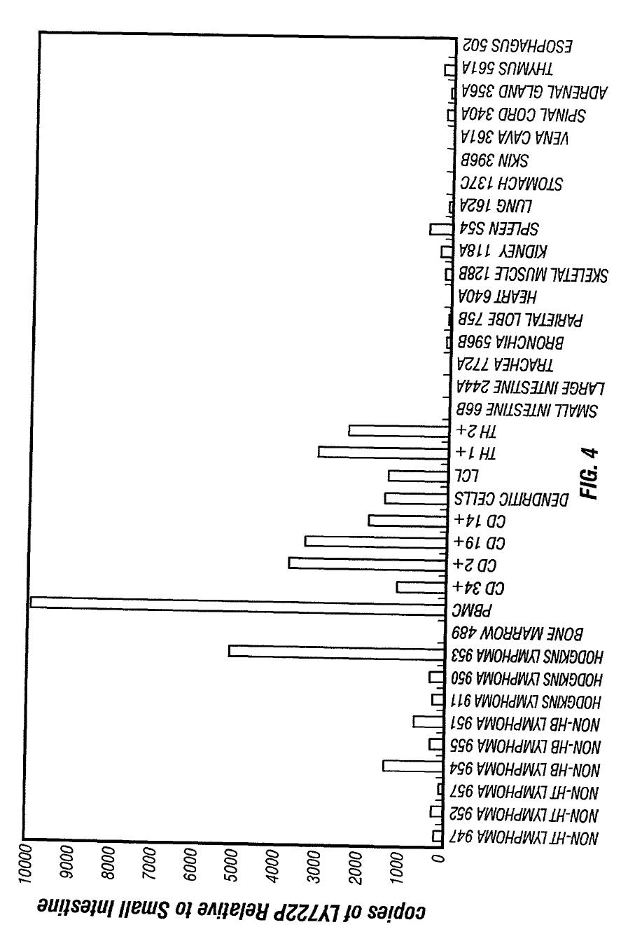

FIG. 5 illustrates the results of TCL extended normal panel.

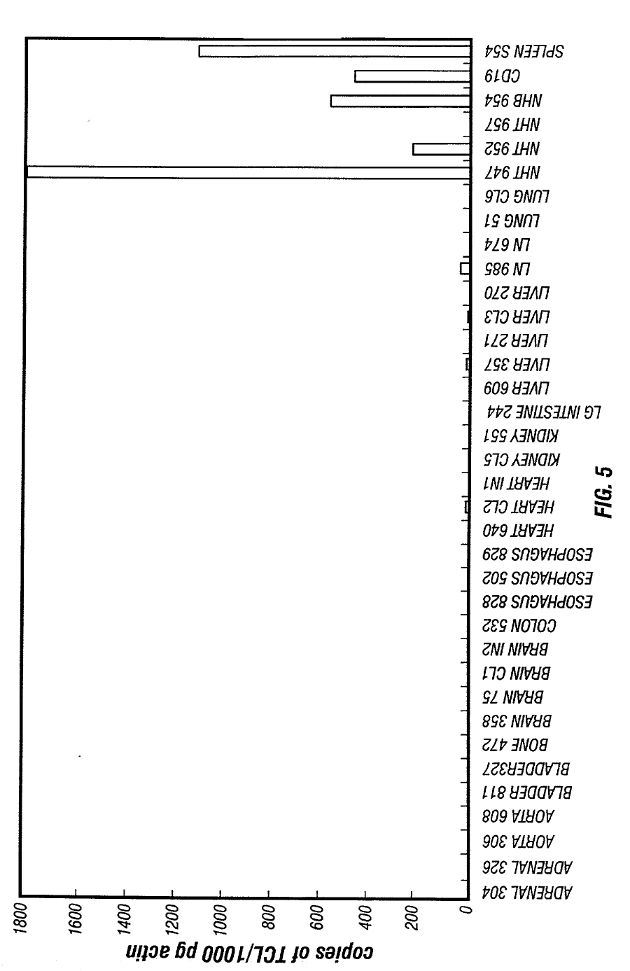

### DESCRIPTION OF ILLUSTRATIVE EMBODIMENTS

In order that the invention herein described may be more fully understood, the following description of various illustrative embodiments is set forth.

The present invention is generally directed to compositions and methods for the immunotherapy and diagnosis of Hematological malignancies, such as leukemias and lymphomas of the Hodgkin's and non-Hodgkin's type.

Methods of Nucleic Acid Delivery and DNA Transfection

In certain embodiments, it is contemplated that one or more RNA or DNA and/or substituted polynucleotide compositions disclosed herein will be used to transfect an appropriate host cell. Technology for introduction of RNAs and DNAs, and vectors comprising them into suitable host cells is well known to those of skill in the art. In particular, such polynucleotides may be used to genetically transform one or more host cells, when therapeutic administration of one or more active peptides, compounds or vaccines is achieved through the expression of one or more polynucleotide constructs that encode one or more therapeutic compounds of interest.

A variety of means for introducing polynucleotides and/or polypeptides into suitable target cells is known to those of skill in the art. For example, when polynucleotides are contemplated for delivery to cells, several non-viral methods for the transfer of expression constructs into cultured mammalian cells are available to the skilled artisan for his use. These include, for example, calcium phosphate precipitation (Graham and Van Der Eb, 1973; Chen and Okayama, 1987; Rippe et al., 1990); DEAE-dextran precipitation (Gopal, 1985); electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

A bacterial cell, a yeast cell, or an animal cell transformed with one or more of the disclosed expression vectors represent an important aspect of the present invention. Such transformed host cells are often desirable for use in the expression of the various DNA gene constructs disclosed herein. In some aspects of the invention, it is often desirable to modulate, regulate, or otherwise control the expression of the gene segments disclosed herein. Such methods are routine to those of skill in the molecular genetic arts. Typically, when increased or over-expression of a particular gene is desired, various manipulations may be employed for enhancing the expression of the messenger RNA, particularly by using an active promoter, and in particular, a tissue-specific promoter such as those disclosed herein, as well as by employing sequences, which enhance the stability of the messenger RNA in the particular transformed host cell.

Typically, the initiation and translational termination region will involve stop codon(s), a terminator region, and optionally, a polyadenylation signal. In the direction of transcription, namely in the 5′ to 3′ direction of the coding or sense sequence, the construct will involve the transcriptional regulatory region, if any, and the promoter, where the regulatory region may be either 5′ or 3′ of the promoter, the ribosomal binding site, the initiation codon, the structural gene having an open reading frame in phase with the initiation codon, the stop codon(s), the polyadenylation signal sequence, if any, and the terminator region. This sequence as a double strand may be used by itself for transformation of a microorganism or eukaryotic host, but will usually be included with a DNA sequence involving a marker, where the second DNA sequence may be joined to the expression construct during introduction of the DNA into the host.

Where no functional replication system is present, the construct will also preferably include a sequence of at least about 30 or about 40 or about 50 basepairs (bp) or so, preferably at least about 60, about 70, about 80, or about 90 to about 100 or so bp, and usually not more than about 500 to about 1000 or so bp of a sequence homologous with a sequence in the host. In this way, the probability of legitimate recombination is enhanced, so that the gene will be integrated into the host and stably maintained by the host. Desirably, the regulatory regions of the expression construct will be in close proximity to (and also operably positioned relative to) the selected therapeutic gene providing for complementation as well as the gene providing for the competitive advantage. Therefore, in the event that the therapeutic gene is lost, the resulting organism will be likely to also lose the gene providing for the competitive advantage, so that it will be unable to compete in the environment with the gene retaining the intact construct.

The selected therapeutic gene can be introduced between the transcriptional and translational initiation region and the transcriptional and translational termination region, so as to be under the regulatory control of the initiation region. This construct may be included in a plasmid, which will include at least one replication system, but may include more than one, where one replication system is employed for cloning during the development of the plasmid and the second replication system is necessary for functioning in the ultimate host, in this case, a mammalian host cell. In addition, one or more markers may be present, which have been described previously. Where integration is desired, the plasmid will desirably include a sequence homologous with the host genome.

Genes or other nucleic acid segments, as disclosed herein, can be inserted into host cells using a variety of techniques that are well known in the art. Five general methods for delivering a nucleic segment into cells have been described: (1) chemical methods (Graham and VanDerEb, 1973); (2) physical methods such as microinjection (Capecchi, 1980), electroporation (U.S. Pat. No. 5,472,869; Wong and Neumann, 1982; Fromm et al., 1985), microprojectile bombardment (U.S. Pat. No. 5,874,265, specifically incorporated herein by reference in its entirety), “gene gun” (Yang et al., 1990); (3) viral vectors (Eglitis and Anderson, 1988); (4) receptor-mediated mechanisms (Curiel et al., 1991; Wagner et al., 1992); and (5) bacterial-mediated transformation.

Hematological Malignancy Related-Specific Antibodies and Antigen-Binding Fragments Thereof

The present invention further provides antibodies and antigen-binding fragments thereof, that specifically bind to (or are immunospecific for) at least a first peptide or peptide variant as disclosed herein. As used herein, an antibody or an antigen-binding fragment is said to “specifically bind” to a peptide if it reacts at a detectable level (within, for example, an ELISA) with the peptide, and does not react detectably with unrelated peptides or proteins under similar conditions. As used herein, “binding” refers to a noncovalent association between two separate molecules such that a “complex” is formed. The ability to bind may be evaluated by, for example, determining a binding constant for the formation of the complex. The binding constant is the value obtained when the concentration of the complex is divided by the product of the component concentrations. In the context of the present invention, in general, two compounds are said to “bind” when the binding constant for complex formation exceeds about 10³ L/mol. The binding constant maybe determined using methods well known in the art.

Any agent that satisfies the above requirements may be a binding agent. In illustrative embodiments, a binding agent is an antibody or an antigen-binding fragment thereof. Such antibodies may be prepared by any of a variety of techniques known to those of ordinary skill in the art (Harlow and Lane, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the peptide is initially injected into any of a wide variety of mammals (e.g., mice, rats, rabbits, sheep or goats). In this step, the peptides of this invention may serve as the immunogen without modification. Alternatively, particularly for relatively short peptides, a superior immune response may be elicited if the peptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the peptide may then be purified from such antisera by, for example, affinity chromatography using the peptide coupled to a suitable solid support.

Monoclonal antibodies specific for the antigenic peptide of interest may be prepared, for example, using the technique of Kohler and Milstein (1976) and improvements thereto. Briefly, these methods involve the preparation of immortal cell lines capable of producing antibodies having the desired specificity (i.e., reactivity with the peptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the peptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The peptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, 1988) and digested by papain to yield Fab and Fc fragments. The Fab and Fc fragments may be separated by affinity chromatography on Protein A bead columns.

Monoclonal antibodies and fragments thereof may be coupled to one or more therapeutic agents. Suitable agents in this regard include radioactive tracers and chemotherapeutic agents, which may be used, for example, to purge autologous bone marrow in vitro). Representative therapeutic agents include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein. For diagnostic purposes, coupling of radioactive agents may be used to facilitate tracing of metastases or to determine the location of hematological malignancy related-positive tumors.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be affected, for example, through amino groups, carboxyl groups, and sulfhydryl groups or oxidized carbohydrate residues. There are numerous references describing such methodology, e.g., U.S. Pat. No. 4,671,958.

Where a therapeutic agent is more potent when free from the antibody portion of the immunoconjugates of the present invention, it may be desirable to use a linker group that is cleavable during or upon internalization into a cell. A number of different cleavable linker groups have been described. The mechanisms for the intracellular release of an agent from these linker groups include cleavage by reduction of a disulfide bond (U.S. Pat. No. 4,489,710), by irradiation of a photolabile bond (U.S. Pat. No. 4,625,014), by hydrolysis of derivatized amino acid side chains (U.S. Pat. No. 4,638,045), by serum complement-mediated hydrolysis (U.S. Pat. No. 4,671,958), and acid-catalyzed hydrolysis (U.S. Pat. No. 4,569,789).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used. A carrier may bear the agents in a variety of ways, including covalent bonding either directly or via a linker group. Suitable carriers include proteins such as albumins (U.S. Pat. No. 4,507,234), peptides and polysaccharides such as aminodextran (U.S. Pat. No. 4,699,784). A carrier may also bear an agent by noncovalent bonding or by encapsulation, such as within a liposome vesicle (U.S. Pat. No. 4,429,008 and U.S. Pat. No. 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562 discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

Also provided herein are anti-idiotypic antibodies that mimic an immunogenic portion of hematological malignancy related. Such antibodies may be raised against an antibody, or an antigen-binding fragment thereof, that specifically binds to an immunogenic portion of hematological malignancy related, using well-known techniques. Anti-idiotypic antibodies that mimic an immunogenic portion of hematological malignancy related are those antibodies that bind to an antibody, or antigen-binding fragment thereof, that specifically binds to an immunogenic portion of hematological malignancy related, as described herein.

Irrespective of the source of the original hematological malignancy related peptide-specific antibody, the intact antibody, antibody multimers, or any one of a variety of functional, antigen-binding regions of the antibody may be used in the present invention. Exemplary functional regions include scFv, Fv, Fab′, Fab and F(ab′)₂ fragments of the hematological malignancy related peptide-specific antibodies. Techniques for preparing such constructs are well known to those in the art and are further exemplified herein.

The choice of antibody construct may be influenced by various factors. For example, prolonged half-life can result from the active readsorption of intact antibodies within the kidney, a property of the Fc piece of immunoglobulin. IgG based antibodies, therefore, are expected to exhibit slower blood clearance than their Fab′ counterparts. However, Fab′ fragment-based compositions will generally exhibit better tissue penetrating capability.

Antibody fragments can be obtained by proteolysis of the whole immunoglobulin by the non-specific thiol protease, papain. Papain digestion yields two identical antigen-binding fragments, termed “Fab fragments,” each with a single antigen-binding site, and a residual “Fc fragment.”

Papain should first be activated by reducing the sulphydryl group in the active site with cysteine, 2-mercaptoethanol or dithiothreitol. Heavy metals in the stock enzyme should be removed by chelation with EDTA (2 mM) to ensure maximum enzyme activity. Enzyme and substrate are normally mixed together in the ratio of 1:100 by weight. After incubation, the reaction can be stopped by irreversible alkylation of the thiol group with iodoacetamide or simply by dialysis. The completeness of the digestion should be monitored by SDS-PAGE and the various fractions separated by Protein A-Sepharose or ion exchange chromatography.

The usual procedure for preparation of F(ab′)₂ fragments from IgG of rabbit and human origin is limited proteolysis by the enzyme pepsin. The conditions, 100×antibody excess wt./wt. in acetate buffer at pH 4.5, 37° C., suggest that antibody is cleaved at the C-terminal side of the inter-heavy-chain disulfide bond. Rates of digestion of mouse IgG may vary with subclass and it may be difficult to obtain high yields of active F(ab′)₂ fragments without some undigested or completely degraded IgG. In particular, IgG_{2b} is highly susceptible to complete degradation. The other subclasses require different incubation conditions to produce optimal results, all of which is known in the art.

Pepsin treatment of intact antibodies yields an F(ab′)₂ fragment that has two antigen-combining sites and is still capable of cross-linking antigen. Digestion of rat IgG by pepsin requires conditions including dialysis in 0.1 M acetate buffer, pH 4.5, and then incubation for four hrs with 1% wt./wt. pepsin; IgG₁ and IgG₂ₐ digestion is improved if first dialyzed against 0.1 M formate buffer, pH 2.8, at 4° C., for 16 hrs followed by acetate buffer. IgG_{2b} gives more consistent results with incubation in staphylococcal V8 protease (3% wt./wt.) in 0.1 M sodium phosphate buffer, pH 7.8, for four hrs at 37° C.

A Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them. Other chemical couplings of antibody fragments are also known.

The term “variable,” as used herein in reference to antibodies, means that certain portions of the variable domains differ extensively in sequence among antibodies, and are used in the binding and specificity of each particular antibody to its particular antigen. However, the variability is not evenly distributed throughout the variable domains of antibodies. It is concentrated in three segments termed “hypervariable regions,” both in the light chain and the heavy chain variable domains.

The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases, forming part of, the β-sheet structure.

The hypervariable regions in each chain are held together in close proximity by the FRs and, with the hypervariable regions from the other chain, contribute to the formation of the antigen-binding site of antibodies (Kabat et al., 1991, specifically incorporated herein by reference). The constant domains are not involved directly in binding an antibody to an antigen, but exhibit various effector functions, such as participation of the antibody in antibody-dependent cellular toxicity.

The term “hypervariable region,” as used herein, refers to the amino acid residues of an antibody that are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-56 (H2) and 95-102 (H3) in the heavy chain variable domain (Kabat et al., 1991, specifically incorporated herein by reference) and/or those residues from a “hypervariable loop” (i.e., residues 26-32 (L1), 50-52(L2) and 91-96 (L3) in the light chain variable domain and 26-32 (H1), 53-55 (H2) and 96-101 (H3) in the heavy chain variable domain). “Framework” or “FR” residues are those variable domain residues other than the hypervariable region residues as herein defined.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the V_{H}-V_{L} dimer. Collectively, six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding.

“Diabodies” are small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described in European Pat. Appl. No. EP 404,097 and Intl. Pat. Appl. Publ. No. WO 93/11161, each specifically incorporated herein by reference. “Linear antibodies”, which can be bispecific or monospecific, comprise a pair of tandem Fd segments (V_{H}-C_{H}1-V_{H}-C_{H}1) that form a pair of antigen binding regions, as described in Zapata et al. (1995), specifically incorporated herein by reference.

Other types of variants are antibodies with improved biological properties relative to the parent antibody from which they are generated. Such variants, or second-generation compounds, are typically substitutional variants involving one or more substituted hypervariable region residues of a parent antibody. A convenient way for generating such substitutional variants is affinity maturation using phage display.

In affinity maturation using phage display, several hypervariable region sites (e.g., 6 to 7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine-scanning mutagenesis can be performed on hypervariable region residues identified as contributing significantly to antigen binding.

Alternatively, or in addition, the crystal structure of the antigen-antibody complex be delineated and analyzed to identify contact points between the antibody and target. Such contact residues and neighboring residues are candidates for substitution. Once such variants are generated, the panel of variants is subjected to screening, and antibodies with analogues but different or even superior properties in one or more relevant assays are selected for further development.

In using a Fab′ or antigen binding fragment of an antibody, with the attendant benefits on tissue penetration, one may derive additional advantages from modifying the fragment to increase its half-life. A variety of techniques may be employed, such as manipulation or modification of the antibody molecule itself, and also conjugation to inert carriers. Any conjugation for the sole purpose of increasing half-life, rather than to deliver an agent to a target, should be approached carefully in that Fab′ and other fragments are chosen to penetrate tissues. Nonetheless, conjugation to non-protein polymers, such PEG and the like, is contemplated.

Modifications other than conjugation are therefore based upon modifying the structure of the antibody fragment to render it more stable, and/or to reduce the rate of catabolism in the body. One mechanism for such modifications is the use of D-amino acids in place of L-amino acids. Those of ordinary skill in the art will understand that the introduction of such modifications needs to be followed by rigorous testing of the resultant molecule to ensure that it still retains the desired biological properties. Further stabilizing modifications include the use of the addition of stabilizing moieties to either the N-terminal or the C-terminal, or both, which is generally used to prolong the half-life of biological molecules. By way of example only, one may wish to modify the termini by acylation or amination.

Moderate conjugation-type modifications for use with the present invention include incorporating a salvage receptor binding epitope into the antibody fragment. Techniques for achieving this include mutation of the appropriate region of the antibody fragment or incorporating the epitope as a peptide tag that is attached to the antibody fragment. Intl. Pat. Appl. Publ. No. WO 96/32478 is specifically incorporated herein by reference for the purposes of further exemplifying such technology. Salvage receptor binding epitopes are typically regions of three or more amino acids from one or two lops of the Fc domain that are transferred to the analogous position on the antibody fragment. The salvage receptor-binding epitopes disclosed in Intl. Pat. Appl. Publ. No. WO 98/45331 are incorporated herein by reference for use with the present invention.

T Cell Compositions Specific for Hematological Malignancy-Related Peptides

Immunotherapeutic compositions may also, or alternatively, comprise T cells specific for hematological malignancy related. Such cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system, such as the Isolex™ System, available from Nexell Therapeutics, Inc. (Irvine, Calif.; see also U.S. Pat. Nos. 5,240,856; 5,215,926; Intl. Pat. Appl. Publ. No. WO 89/06280; Intl. Pat. Appl. Publ. No. WO 91/16116 and Intl. Pat. Appl. Publ. No. WO 92/07243). Alternatively, T cells may be derived from related or unrelated humans, non-human mammals, cell lines or cultures.

T cells may be stimulated with hematological malignancy related peptide, polynucleotide encoding a hematological malignancy related peptide and/or an antigen-presenting cell (APC) that expresses a hematological malignancy related peptide. Such stimulation is performed under conditions and for a time sufficient to permit the generation of T cells that are specific for the hematological malignancy related peptide. Preferably, a hematological malignancy related peptide or polynucleotide is present within a delivery vehicle, such as a microsphere, to facilitate the generation of antigen-specific T cells. Briefly, T cells, which may be isolated from a patient or a related or unrelated donor by routine techniques (such as by Ficoll/Hypaque® density gradient centrifugation of peripheral blood lymphocytes), are incubated with hematological malignancy related peptide. For example, T cells may be incubated in vitro for 2-9 days (typically 4 days) at 37° C. with hematological malignancy related peptide (e.g., 5 to 25 μg/ml) or cells synthesizing a comparable amount of hematological malignancy related peptide. It may be desirable to incubate a separate aliquot of a T cell sample in the absence of hematological malignancy related peptide to serve as a control.

T cells are considered to be specific for a hematological malignancy related peptide if the T cells kill target cells coated with a hematological malignancy related peptide or expressing a gene encoding such a peptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al. (1994). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a hematological malignancy related peptide may be quantified. Contact with a hematological malignancy related peptide (200 ng/ml-100 μg/ml, preferably 100 ng/ml-25 μg/ml) for 3-7 days should result in at least a two-fold increase in proliferation of the T cells and/or contact as described above for 2-3 hrs should result in activation of the T cells, as measured using standard cytokine assays in which a two-fold increase in the level of cytokine release (e.g., TNF or IFN-γ) is indicative of T cell activation (Coligan et al., 1998). hematological malignancy related specific T cells may be expanded using standard techniques. Within preferred embodiments, the T cells are derived from a patient or a related or unrelated donor and are administered to the patient following stimulation and expansion.

T cells that have been activated in response to a hematological malignancy related peptide, polynucleotide or hematological malignancy related-expressing APC may be CD4⁺ and/or CD8⁺. Specific activation of CD4⁺ or CD8⁺ T cells may be detected in a variety of ways. Methods for detecting specific T cell activation include detecting the proliferation of T cells, the production of cytokines (e.g., lymphokines), or the generation of cytolytic activity (i.e., generation of cytotoxic T cells specific for hematological malignancy related). For CD4⁺ T cells, a preferred method for detecting specific T cell activation is the detection of the proliferation of T cells. For CD8⁺ T cells, a preferred method for detecting specific T cell activation is the detection of the generation of cytolytic activity.

For therapeutic purposes, CD4⁺ or CD8⁺ T cells that proliferate in response to the hematological malignancy related peptide, polynucleotide or APC can be expanded in number either in vitro or in vivo. Proliferation of such T cells in vitro may be accomplished in a variety of ways. For example, the T cells can be re-exposed to hematological malignancy related peptide, with or without the addition of T cell growth factors, such as interleukin-2, and/or stimulator cells that synthesize a hematological malignancy related peptide. The addition of stimulator cells is preferred where generating CD8⁺ T cell responses. T cells can be grown to large numbers in vitro with retention of specificity in response to intermittent restimulation with hematological malignancy related peptide. Briefly, for the primary in vitro stimulation (IVS), large numbers of lymphocytes (e.g., greater than 4×10⁷) may be placed in flasks with media containing human serum. hematological malignancy related peptide (e.g., peptide at 10 μg/ml) may be added directly, along with tetanus toxoid (e.g., 5 μg/ml). The flasks may then be incubated (e.g., 37° C. for 7 days). For a second IVS, T cells are then harvested and placed in new flasks with 2-3×10⁷ irradiated peripheral blood mononuclear cells. hematological malignancy related peptide (e.g., 10 μg/ml) is added directly. The flasks are incubated at 37° C. for 7 days. On day 2 and day 4 after the second IVS, 2-5 units of interleukin-2 (IL-2) may be added. For a third IVS, the T cells may be placed in wells and stimulated with the individual's own EBV transformed B cells coated with the peptide. IL-2 may be added on days 2 and 4 of each cycle. As soon as the cells are shown to be specific cytotoxic T cells, they may be expanded using a 10-day stimulation cycle with higher IL-2 (20 units) on days 2, 4 and 6.

Alternatively, one or more T cells that proliferate in the presence of hematological malignancy related peptide can be expanded in number by cloning. Methods for cloning cells are well known in the art, and include limiting dilution. Responder T cells may be purified from the peripheral blood of sensitized patients by density gradient centrifugation and sheep red cell rosetting and established in culture by stimulating with the nominal antigen in the presence of irradiated autologous filler cells. In order to generate CD4⁺ T cell lines, hematological malignancy related peptide is used as the antigenic stimulus and autologous peripheral blood lymphocytes (PBL) or lymphoblastoid cell lines (LCL) immortalized by infection with Epstein Barr virus are used as antigen-presenting cells. In order to generate CD8⁺ T cell lines, autologous antigen-presenting cells transfected with an expression vector that produces hematological malignancy related peptide may be used as stimulator cells. Established T cell lines may be cloned 2-4 days following antigen stimulation by plating stimulated T cells at a frequency of 0.5 cells per well in 96-well flat-bottom plates with 1×10⁶ irradiated PBL or LCL cells and recombinant interleukin-2 (rIL2) (50 U/ml). Wells with established clonal growth may be identified at approximately 2-3 weeks after initial plating and restimulated with appropriate antigen in the presence of autologous antigen-presenting cells, then subsequently expanded by the addition of low doses of rIL2 (10 U/ml) 2-3 days following antigen stimulation. T cell clones may be maintained in 24-well plates by periodic restimulation with antigen and rIL2 approximately every two weeks. Cloned and/or expanded cells may be administered back to the patient as described, for example, by Chang et al., (1996).

Within certain embodiments, allogeneic T-cells may be primed (i.e., sensitized to hematological malignancy related) in vivo and/or in vitro. Such priming may be achieved by contacting T cells with a hematological malignancy related peptide, a polynucleotide encoding such a peptide or a cell producing such a peptide under conditions and for a time sufficient to permit the priming of T cells. In general, T cells are considered to be primed if, for example, contact with a hematological malignancy related peptide results in proliferation and/or activation of the T cells, as measured by standard proliferation, chromium release and/or cytokine release assays as described herein. A stimulation index of more than two fold increase in proliferation or lysis, and more than three fold increase in the level of cytokine, compared to negative controls indicates T-cell specificity. Cells primed in vitro may be employed, for example, within bone marrow transplantation or as donor lymphocyte infusion.

T cells specific for hematological malignancy related can kill cells that express hematological malignancy related protein. Introduction of genes encoding T-cell receptor (TCR) chains for hematological malignancy related are used as a means to quantitatively and qualitatively improve responses to hematological malignancy related bearing leukemia and cancer cells. Vaccines to increase the number of T cells that can react to hematological malignancy related positive cells are one method of targeting hematological malignancy related bearing cells. T cell therapy with T cells specific for hematological malignancy related is another method. An alternative method is to introduce the TCR chains specific for hematological malignancy related into T cells or other cells with lytic potential. In a suitable embodiment, the TCR alpha and beta chains are cloned out from a hematological malignancy related specific T cell line and used for adoptive T cell therapy, such as described in WO96/30516, incorporated herein by reference.

Pharmaceutical Compositions and Vaccine Formulations

Within certain aspects, peptides, polynucleotides, antibodies and/or T cells may be incorporated into pharmaceutical compositions or immunogenic compositions (i.e., vaccines). Alternatively, a pharmaceutical composition may comprise an antigen-presenting cell (e.g., a dendritic cell) transfected with a hematological malignancy related polynucleotide such that the antigen-presenting cell expresses a hematological malignancy related peptide. Pharmaceutical compositions comprise one or more such compounds or cells and a physiologically acceptable carrier or excipient. Vaccines may comprise one or more such compounds or cells and an immunostimulant, such as an adjuvant or a liposome (into which the compound is incorporated). An immunostimulant may be any substance that enhances or potentiates an immune response (antibody- and/or cell-mediated) to an exogenous antigen. Examples of immunostimulants include adjuvants, biodegradable microspheres (e.g., polylactic galactide) and liposomes (into which the compound is incorporated) (U.S. Pat. No. 4,235,877). Vaccine preparation is generally described in, for example, Powell and Newman (1995). Pharmaceutical compositions and vaccines within the scope of the present invention may also contain other compounds, which may be biologically active or inactive. For example, one or more immunogenic portions of other tumor antigens may be present, either incorporated into a fusion peptide or as a separate compound, within the composition or vaccine.

Within certain embodiments, pharmaceutical compositions and vaccines are designed to elicit T cell responses specific for a hematological malignancy related peptide in a patient, such as a human. In general, T cell responses may be favored through the use of relatively short peptides (e.g., comprising less than 23 consecutive amino acid residues of a native hematological malignancy related peptide, preferably 4-16 consecutive residues, more preferably 8-16 consecutive residues and still more preferably 8-10 consecutive residues). Alternatively, or in addition, a vaccine may comprise an immunostimulant that preferentially enhances a T cell response. In other words, the immunostimulant may enhance the level of a T cell response to a hematological malignancy related peptide by an amount that is proportionally greater than the amount by which an antibody response is enhanced. For example, when compared to a standard oil based adjuvant, such as CFA, an immunostimulant that preferentially enhances a T cell response may enhance a proliferative T cell response by at least two fold, a lytic response by at least 10%, and/or T cell activation by at least two fold compared to hematological malignancy related-negative control cell lines, while not detectably enhancing an antibody response. The amount by which a T cell or antibody response to a hematological malignancy related peptide is enhanced may generally be determined using any representative technique known in the art, such as the techniques provided herein.

A pharmaceutical composition or vaccine may contain DNA encoding one or more of the peptides as described above, such that the peptide is generated in situ. As noted above, the DNA may be present within any of a variety of delivery systems known to those of ordinary skill in the art, including nucleic acid expression systems, bacterial and viral expression systems and mammalian expression systems. Numerous gene delivery techniques are well known in the art (Rolland, 1998, and references cited therein). Appropriate nucleic acid expression systems contain the necessary DNA, cDNA or RNA sequences for expression in the patient (such as a suitable promoter and terminating signal). Bacterial delivery systems involve the administration of a bacterium (such as Bacillus-Calmette-Guerrin) that expresses an immunogenic portion of the peptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus (Fisher-Hoch et al., 1989; Flexner et al., 1989; Flexner et al., 1990; U.S. Pat. Nos. 4,603,112, 4,769,330, 5,017,487; Intl. Pat. Appl. Publ. No. WO 89/01973; U.S. Pat. No. 4,777,127; Great Britain Pat. No. GB 2,200,651; European Pat. No. EP 0,345,242; Intl. Pat. Appl. Publ. No. WO 91/02805; Berkner, 1988; Rosenfeld et al., 1991; Kolls et al., 1994; Kass-Eisler et al., 1993; Guzman et al., 1993a; and Guzman et al., 1993). Techniques for incorporating DNA into such expression systems are well known to those of ordinary skill in the art. The DNA may also be “naked,” as described, for example, in Ulmer et al. (1993) and reviewed by Cohen (1993). The uptake of naked DNA may be increased by coating the DNA onto biodegradable beads, which are efficiently transported into the cells. It will be apparent that a vaccine may comprise both a polynucleotide and a peptide component. Such vaccines may provide for an enhanced immune response.

As noted above, a pharmaceutical composition or vaccine may comprise an antigen-presenting cell that expresses a hematological malignancy related peptide. For therapeutic purposes, as described herein, the antigen-presenting cell is preferably an autologous dendritic cell. Such cells may be prepared and transfected using standard techniques (Reeves et al., 1996; Tuting et al., 1998; and Nair et al., 1998). Expression of a hematological malignancy related peptide on the surface of an antigen-presenting cell may be confirmed by in vitro stimulation and standard proliferation as well as chromium release assays, as described herein.

It will be apparent to those of ordinary skill in the art having the benefit of the present teachings that a vaccine may contain pharmaceutically acceptable salts of the polynucleotides and peptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts). The phrases “pharmaceutically or pharmacologically acceptable” refer to molecular entities and compositions that do not produce an adverse, allergic or other significant untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, and general safety and purity standards as required by the Food and Drug Administration Office of Biologics standards. Supplementary active ingredients can also be incorporated into the compositions.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. For certain topical applications, formulation as a cream or lotion, using well-known components, is preferred.

Such compositions may also comprise buffers (e.g., neutral buffered saline or phosphate buffered saline), carbohydrates (e.g., glucose, mannose, sucrose or dextrans), mannitol, proteins, peptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate, or formulated with one or more liposomes, microspheres, nanoparticles, or micronized delivery systems using well-known technology.

Any of a variety of immunostimulants, such as adjuvants, may be employed in the preparation of vaccine compositions of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, alum-based adjuvants (e.g., Alhydrogel, Rehydragel, aluminum phosphate, Algammulin, aluminum hydroxide); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, may also be used as adjuvants.

Hemocyanins and hemoerythrins may also be used in the invention. The use of hemocyanin from keyhole limpet (KLH) is particularly preferred, although other molluscan and arthropod hemocyanins and hemoerythrins may be employed. Various polysaccharide adjuvants may also be used. Polyamine varieties of polysaccharides are particularly preferred, such as chitin and chitosan, including deacetylated chitin.

A further preferred group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide that is proposed for use in artificial liposomes formed from phosphatidyl choline and phosphatidyl glycerol. It is said to be effective in activating human monocytes and destroying tumor cells, but is non-toxic in generally high doses. The compounds of U.S. Pat. No. 4,950,645, and Intl. Pat. Appl. Publ. No. WO 91/16347 are also proposed for use in achieving particular aspects of the present invention.

BCG and BCG-cell wall skeleton (CWS) may also be used as adjuvants in the invention, with or without trehalose dimycolate. Trehalose dimycolate may be used itself. Azuma et al. (1988) show that trehalose dimycolate administration correlates with augmented resistance to influenza virus infection in mice. Trehalose dimycolate may be prepared as described in U.S. Pat. No. 4,579,945.

Amphipathic and surface-active agents, e.g., saponin and derivatives such as QS21 (Cambridge Biotech), form yet another group of preferred adjuvants for use with the immunogens of the present invention. Nonionic block copolymer surfactants (Rabinovich et al., 1994; Hunter et al., 1991) may also be employed. Oligonucleotides, as described by Yamamoto et al. (1988) are another useful group of adjuvants. Quil A and lentinen are also preferred adjuvants.

Superantigens are also contemplated for use as adjuvants in the present invention. “Superantigens” are generally bacterial products that stimulate a greater proportion of T lymphocytes than peptide antigens without a requirement for antigen processing (Mooney et. al., 1994). Superantigens include Staphylococcus exoproteins, such as the α, β, γ and δ enterotoxins from S. aureus and S. epidermidis, and the α, β, γ and δ E. coli exotoxins.

Common Staphylococcus enterotoxins are known as staphylococcal enterotoxin A (SEA) and staphylococcal enterotoxin B (SEB), with enterotoxins through E (SEE) being described (Rott et. al, 1992). Streptococcus pyogenes B (SEB), Clostridium perfringens enterotoxin (Bowness et. al., 1992), cytoplasmic membrane-associated protein (CAP) from S. pyogenes (Sato et. al., 1994) and toxic shock syndrome toxin-i (TSST-1) from S. aureus (Schwab et. al., 1993) are further useful superantigens.

One group of adjuvants particularly preferred for use in the invention are the detoxified endotoxins, such as the refined detoxified endotoxin of U.S. Pat. No. 4,866,034. These refined detoxified endotoxins are effective in producing adjuvant responses in mammals.

The detoxified endotoxins may be combined with other adjuvants. Combination of detoxified endotoxins with trehalose dimycolate is contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins are also envisioned to be useful, as described in U.S. Pat. No. 4,520,019.

MPL is currently one preferred immunopotentiating agent for use herein. References that concern the uses of MPL include Tomai et al. (1987), Chen et l. (1991) and Garg and Subbarao (1992), that each concern certain roles of MPL in the reactions of aging mice; Elliott et al. (1991), that concerns the D-galactosamine loaded mouse and its enhanced sensitivity to lipopolysaccharide and MPL; Chase et al. (1986), that relates to bacterial infections; and Masihi et al. (1988), that describes the effects of MPL and endotoxin on resistance of mice to Toxoplasma gondii. Fitzgerald (1991) also reported on the use of MPL to up-regulate the immunogenicty of a syphilis vaccine and to confer significant protection against challenge infection in rabbits.

Thus MPL is known to be safe for use, as shown in the above model systems. Phase-I clinical trials have also shown MPL to be safe for use (Vosika et al., 1984). Indeed, 100 μg/m² is known to be safe for human use, even on an outpatient basis (Vosika et al., 1984).

MPL generally induces polyclonal B cell activation (Baker et al., 1994), and has been shown to augment antibody production in many systems, for example, in immunologically immature mice (Baker et al., 1988); in aging mice (Tomai and Johnson, 1989); and in nude and Xid mice (Madonna and Vogel, 1986; Myers et al., 1995). Antibody production has been shown against erythrocytes (Hraba et al., 1993); T cell dependent and independent antigens; Pnu-immune vaccine (Garg and Subbarao, 1992); isolated tumor-associated antigens (U.S. Pat. No. 4,877,611); against syngeneic tumor cells (Livingston et al., 1985; Ravindranath et al., 1994a;b); and against tumor-associated gangliosides (Ravindranath et al., 1994a;b).

Another useful attribute of MPL is that is augments IgM responses, as shown by Baker et al. (1988a), who describe the ability of MPL to increase antibody responses in young mice. This is a particularly useful feature of an adjuvant for use in certain embodiments of the present invention. Myers et al. (1995) recently reported on the ability of MPL to induce IgM antibodies, by virtue T cell-independent antibody production.

In the Myers et al. (1995) studies, MPL was conjugated to the hapten, TNP. MPL was proposed for use as a carrier for other haptens, such as peptides.

MPL also activates and recruits macrophages (Verna et al., 1992). Tomai and Johnson (1989) showed that MPL-stimulated T cells enhance IL-1 secretion by macrophages. MPL is also known to activate superoxide production, lysozyme activity, phagocytosis, and killing of Candida in murine peritoneal macrophages (Chen et al., 1991).

The effects of MPL on T cells include the endogenous production of cytotoxic factors, such as TNF, in serum of BCG-primed mice by MPL (Bennett et al., 1988). Kovach et al. (1990) and Elliot et al. (1991) also show that MPL induces TNF activity. MPL is known to act with TNF-α to induce release of IFN-γ by NK cells. IFN-γ production by T cells in response to MPL was also documented by Tomai and Johnson (1989), and Odean et al. (1990).

MPL is also known to be a potent T cell adjuvant. For example, MPL stimulates proliferation of melanoma-antigen specific CTLs (Mitchell et al., 1988, 1993). Further, Baker et al. (1988b) showed that nontoxic MPL inactivated suppressor T cell activity. Naturally, in the physiological environment, the inactivation of T suppressor cells allows for increased benefit for the animal, as realized by, e.g., increased antibody production. Johnson and Tomai (1988) have reported on the possible cellular and molecular mediators of the adjuvant action of MPL.

MPL is also known to induce aggregation of platelets and to phosphorylate a platelet protein prior to induction of serotonin secretion (Grabarek et al., 1990). This study shows that MPL is involved in protein kinase C activation and signal transduction.

Many articles concern the structure and function of MPL include. These include Johnson et al. (1990), that describes the structural characterization of MPL homologs obtained from Salmonella minnesota Re595 lipopolysaccharide. The work of Johnson et al. (1990), in common with Grabarek et al. (1990), shows that the fatty acid moieties of MPL can vary, even in commercial species. In separating MPL into eight fractions by thin layer chromatography, Johnson et al. (1990) found that three were particularly active, as assessed using human platelet responses. The chemical components of the various MPL species were characterized by Johnson et al. (1990).

Baker et al. (1992) further analyzed the structural features that influence the ability of lipid A and its analogs to abolish expression of suppressor T cell activity. They reported that decreasing the number of phosphate groups in lipid A from two to one (i.e., creating monophosphoryl lipid A, MPL) as well as decreasing the fatty acyl content, primarily by removing the residue at the 3 position, resulted in a progressive reduction in toxicity; however, these structural modifications did not influence its ability to abolish the expression of Ts function (Baker et al., 1992). These types of MPL are ideal for use in the present invention.

Baker et al. (1992) also showed that reducing the fatty acyl content from five to four (lipid A precursor IV_{A} or Iₐ) eliminated the capacity to influence Ts function but not to induce polyclonal activation of B cells. These studies show that in order to be able to abolish the expression of Ts function, lipid A must be a glucosamine disaccharide; may have either one or two phosphate groups; and must have at least five fatty acyl groups. Also, the chain length of the nonhydroxylated fatty acid, as well as the location of acyloxyacyl groups (2′ versus 3′ position), may play an important role (Baker et al., 1992).

In examining the relationship between chain length and position of fatty acyl groups on the ability of lipid A to abolish the expression of suppressor T-cell (Ts) activity, Baker et al. (1994) found that fatty acyl chain lengths of C₁₂ to C₁₄ appeared to be optimal for bioactivity. Therefore, although their use is still possible, lipid A preparations with fatty acyl groups of relatively short chain length (C₁₀ to C₁₂ from Pseudomonas aeruginosa and Chromobacterium violaceum) or predominantly long chain length (C₁₈ from Helicobacter pylori) are less preferred for use in this invention.

Baker et al. (1994) also showed that the lipid A proximal inner core region oligosaccharides of some bacterial lipopolysaccharides increase the expression of Ts activity; due mainly to the capacity of such oligosaccharides, which are relatively conserved in structure among gram-negative bacterial, to enlarge or expand upon the population of CD8⁺ Ts generated during the course of a normal antibody response to unrelated microbial antigens. The minimal structure required for the expression of the added immunosuppression observed was reported to be a hexasaccharide containing one 2-keto-3-deoxyoctonate residue, two glucose residues, and three heptose residues to which are attached two pyrophosphorylethanolamine groups (Baker et al., 1994). This information may be considered in utilizing or even designing further adjuvants for use in the invention.

In a generally related line of work, Tanamoto et al. (1994a;b; 1995) described the dissociation of endotoxic activities in a chemically synthesized Lipid A precursor after acetylation or succinylation. Thus, compounds such as “acetyl 406” and “succinyl 516” (Tanamoto et al., 1994a;b; 1995) are also contemplated for use in the invention.

Synthetic MPLs form a particularly preferred group of antigens. For example, Brade et al. (1993) described an artificial glycoconjugate containing the bisphosphorylated glucosamine disaccharide backbone of lipid A that binds to anti-Lipid A MAbs. This is one candidate for use in certain aspects of the invention.

The MPL derivatives described in U.S. Pat. No. 4,987,237 are particularly contemplated for use in the present invention. U.S. Pat. No. 4,987,237 describes MPL derivatives that contain one or more free groups, such as amines, on a side chain attached to the primary hydroxyl groups of the monophosphoryl lipid A nucleus through an ester group. The derivatives provide a convenient method for coupling the lipid A through coupling agents to various biologically active materials. The immunostimulant properties of lipid A are maintained. All MPL derivatives in accordance with U.S. Pat. No. 4,987,237 are envisioned for use in the MPL adjuvant-incorporated cells of this invention.

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines see e.g., Mosmann and Coffman (1989).

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see e.g., U.S. Pat. Nos. 4,436,727; 4,877,611; 4,866,034 and 4,912,094, each of which is specifically incorporated herein by reference in its entirety). CpG-containing oligonucleotides (in which the CpG dinucleotide is unmethylated) also induce a predominantly Th1 response. Such oligonucleotides are well known and are described, for example, in Intl. Pat. Appl. Publ. No. WO 96/02555 and Intl. Pat. Appl. Publ. No. WO 99/33488. Immunostimulatory DNA sequences are also described, for example, by Sato et al. (1996). Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL (see e.g., Intl. Pat. Appl. Publ. No. WO 94/00153), or a less reactogenic composition where the QS21 is quenched with cholesterol (see e.g., Intl. Pat. Appl. Publ. No. WO 96/33739). Other preferred formulations comprise an oil-in-water emulsion and tocopherol. A particularly potent adjuvant formulation involving QS21, 3D-MPL and tocopherol in an oil-in-water emulsion has also been described (see e.g., Intl. Pat. Appl. Publ. No. WO 95/17210).

Other preferred adjuvants include Montanide ISA 720 (Seppic), SAF (Chiron), ISCOMS (CSL), MF-59 (Chiron), the SBAS series of adjuvants (e.g., SBAS-2 or SBAS-4, available from SmithKline Beecham, Rixensart, Belgium), Detox (Corixa Corporation), RC-529 (Corixa Corporation) and aminoalkyl glucosaminide 4-phosphates (AGPs).

Any vaccine provided herein may be prepared using well-known methods that result in a combination of one or more antigens, one or more immunostimulants or adjuvants and one or more suitable carriers, excipients, or pharmaceutically acceptable buffers. The compositions described herein may be administered as part of a sustained release formulation (i.e., a formulation such as a capsule, sponge or gel [composed of polysaccharides, for example] that effects a slow release of compound following administration). Such formulations may generally be prepared using well-known technology (Coombes et al., 1996) and administered by, for example, oral, rectal or subcutaneous implantation, or by implantation at the desired target site. Sustained-release formulations may contain a peptide, polynucleotide or antibody dispersed in a carrier matrix and/or contained within a reservoir surrounded by a rate-controlling membrane.

Carriers for use within such formulations are preferably biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (U.S. Pat. No. 5,151,254; Intl. Pat. Appl. Publ. No. WO 94/20078; Intl. Pat. Appl. Publ. No. WO/94/23701; and Intl. Pat. Appl. Publ. No. WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, 1998) and have been shown to be effective as a physiological adjuvant for eliciting prophylactic or therapeutic antitumor immunity (Timmerman and Levy, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al., 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a hematological malignancy related peptide, such that the peptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen-presenting cell may be administered to a patient, resulting in transfection that occurs in vivo. In vivo and ex vivo transfection of dendritic cells, for example, may generally be performed using any methods known in the art, such as those described in Intl. Pat. Appl. Publ. No. WO 97/24447, or the gene gun approach described by Mahvi et al. (1997). Antigen loading of dendritic cells may be achieved by incubating dendritic cells or progenitor cells with the hematological malignancy related peptide, DNA (naked or within a plasmid vector) or RNA; or with antigen-expressing recombinant bacterium or viruses (e.g., vaccinia, fowlpox, adenovirus or lentivirus vectors). Prior to loading, the peptide may be covalently conjugated to an immunological partner that provides T cell help (e.g., a carrier molecule). Alternatively, a dendritic cell may be pulsed with a non-conjugated immunological partner, separately or in the presence of the peptide.

Combined therapeutics is also contemplated, and the same type of underlying pharmaceutical compositions may be employed for both single and combined medicaments. Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Diagnostic and Prognostic Methods for Hematological Malignancy Diseases

The present invention further provides methods for detecting a malignant disease associated with one or more of the polypeptide or polynucleotide compositions disclosed herein, and for monitoring the effectiveness of an immunization or therapy for such a disease. To determine the presence or absence of a malignant disease associated with one or more of the polypeptide or polynucleotide compositions disclosed herein, a patient may be tested for the level of T cells specific for one or more of such compositions. Within certain methods, a biological sample comprising CD4⁺ and/or CD8⁺ T cells isolated from a patient is incubated with one or more of the polypeptide or polynucleotide compositions disclosed herein, and/or an APC that expresses one or more of such peptides or polypeptides, and the presence or absence of specific activation of the T cells is detected, as described herein. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with one or more of the disclosed peptide, polypeptide or polynucleotide compositions (e.g., 5-25 μg/ml). It may be desirable to incubate another aliquot of a T cell sample in the absence of the composition to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a malignant disease associated with expression or one or more of the disclosed polypeptide or polynucleotide compositions. Further correlation may be made, using methods well known in the art, between the level of proliferation and/or cytolytic activity and the predicted response to therapy. In particular, patients that display a higher antibody, proliferative and/or lytic response may be expected to show a greater response to therapy.

Within other methods, a biological sample obtained from a patient is tested for the level of antibody specific for one or more of the hematological malignancy-related peptides or polypeptide s disclosed herein. The biological sample is incubated with hematological malignancy-related peptide or polypeptide, or a polynucleotide encoding such a peptide or polypeptide, and/or an APC that expresses such a peptide or polypeptide under conditions and for a time sufficient to allow immunocomplexes to form. Immunocomplexes formed between the selected peptide or polypeptide and antibodies in the biological sample that specifically bind to the selected peptide or polypeptide are then detected. A biological sample for use within such methods may be any sample obtained from a patient that would be expected to contain antibodies. Suitable biological samples include blood, sera, ascites, bone marrow, pleural effusion, and cerebrospinal fluid.

The biological sample is incubated with the selected peptide or polypeptide in a reaction mixture under conditions and for a time sufficient to permit immunocomplexes to form between the selected peptide or polypeptide and antibodies that are immunospecific for such a peptide or polypeptide. For example, a biological sample and a selected peptide or polypeptide peptide may be incubated at 4° C. for 24-48 hrs.

Following the incubation, the reaction mixture is tested for the presence of immunocomplexes. Detection of immunocomplexes formed between the selected peptide or polypeptide and antibodies present in the biological sample may be accomplished by a variety of known techniques, such as radioimmunoassays (RIA) and enzyme linked immunosorbent assays (ELISA). Suitable assays are well known in the art and are amply described in the scientific and patent literature (Harlow and Lane, 1988). Assays that may be used include, but are not limited to, the double monoclonal antibody sandwich immunoassay technique (U.S. Pat. No. 4,376,110); monoclonal-polyclonal antibody sandwich assays (Wide et al., 1970); the “western blot” method (U.S. Pat. No. 4,452,901); immunoprecipitation of labeled ligand (Brown et al., 1980); enzyme-linked immunosorbent assays (Raines and Ross, 1982); immunocytochemical techniques, including the use of fluorochromes (Brooks et al., 1980); and neutralization of activity (Bowen-Pope et al., 1984). Other immunoassays include, but are not limited to, those described in U.S. Pat. Nos. 3,817,827; 3,850,752; 3,901,654; 3,935,074; 3,984,533; 3,996,345; 4,034,074; and 4,098,876.

For detection purposes, the selected peptide or polypeptide may either be labeled or unlabeled. Unlabeled polypeptide peptide may be used in agglutination assays or in combination with labeled detection reagents that bind to the immunocomplexes (e.g., anti-immunoglobulin, protein G, Protein A or a lectin and secondary antibodies, or antigen-binding fragments thereof, capable of binding to the antibodies that specifically bind to the selected hematological maliganacy-related peptide or polypeptide). If the selected peptide or polypeptide is labeled, the reporter group may be any suitable reporter group known in the art, including radioisotopes, fluorescent groups, luminescent groups, enzymes, biotin and dye particles.

Within certain assays, unlabeled peptide or polypeptide is immobilized on a solid support. The solid support may be any material known to those of ordinary skill in the art to which the peptide may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The peptide may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the antigen and functional groups on the support or may be a linkage by way of a cross-linking agent). Immobilization by adsorption to a well in a microtiter plate or to a membrane is preferred. In such cases, adsorption may be achieved by contacting the selected peptide or polypeptide, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of peptide ranging from about 10 ng to about 10 μg, and preferably about 100 ng to about 1 μg, is sufficient to immobilize an adequate amount of peptide.

Following immobilization, the remaining protein binding sites on the support are typically blocked. Any suitable blocking agent known to those of ordinary skill in the art, such as bovine serum albumin, Tween™ 20™ (Sigma Chemical Co., St. Louis, Mo.), heat-inactivated normal goat serum (NGS), or BLOTTO (buffered solution of nonfat dry milk which also contains a preservative, salts, and an antifoaming agent) may be used. The support is then incubated with a biological sample suspected of containing specific antibody. The sample can be applied neat, or, more often, it can be diluted, usually in a buffered solution which contains a small amount (0.1%-5.0% by weight) of protein, such as BSA, NGS, or BLOTTO. In general, an appropriate contact time (i.e., incubation time) is a period of time that is sufficient to detect the presence of antibody or an antigen binding fragment that is immunospecific for the selected peptide or polypeptide within a sample containing such an antibody or binding fragment thereof. Preferably, the contact time is sufficient to achieve a level of binding that is at least about 95% of that achieved at equilibrium between bound and unbound antibody or antibody fragment. Those of ordinary skill in the art will recognize that the time necessary to achieve equilibrium may be readily determined by assaying the level of binding that occurs over a period of time. At room temperature, an incubation time of about 30 min is generally sufficient.

Unbound sample may then be removed by washing the solid support with an appropriate buffer, such as PBS containing 0.1% Tween™ 20. A detection reagent that binds to the immunocomplexes and that comprises at least a first detectable labe or “reporter” molecule may then be added. The detection reagent is incubated with the immunocomplex for an amount of time sufficient to detect the bound antibody or antigen binding fragment thereof. An appropriate amount of time may generally be determined by assaying the level of binding that occurs over a period of time. Unbound label or detection reagent is then removed and bound label or detection reagent is detected using a suitable assay or analytical instrument. The method employed for detecting the reporter group depends upon the nature of the reporter group. For radioactive labels, scintillation counting or autoradiographic methods are generally appropriate. Spectroscopic methods may be used to detect dyes, luminescent or chemiluminescent moieties and various chromogens, fluorescent labels and such like. Biotin may be detected using avidin, coupled to a different reporter group (commonly a radioactive or fluorescent group or an enzyme). Enzyme reporter groups (e.g., horseradish peroxidase, β-galactosidase, alkaline phosphatase and glucose oxidase) may generally be detected by the addition of substrate (generally for a specific period of time), followed by spectroscopic or other analysis of the reaction products. Regardless of the specific method employed, a level of bound detection reagent that is at least two fold greater than background (i.e., the level observed for a biological sample obtained from a disease-free individual) indicates the presence of a malignant disease associated with expression of the selected peptide or polypeptide.

In general, methods for monitoring the effectiveness of an immunization or therapy involve monitoring changes in the level of antibodies or T cells specific for the selected peptide or polypeptide in a sample, or in an animal such as a human patient. Methods in which antibody levels are monitored may comprise the steps of: (a) incubating a first biological sample, obtained from a patient prior to a therapy or immunization, with a selected peptide or polypeptide, wherein the incubation is performed under conditions and for a time sufficient to allow immunocomplexes to form; (b) detecting immunocomplexes formed between the selected peptide or polypeptide and antibodies or antigen binding fragments in the biological sample that specifically bind to the selected peptide or polypeptide; (c) repeating steps (a) and (b) using a second biological sample taken from the patient at at later time, such as for example, following a given therapy or immunization; and (d) comparing the number of immunocomplexes detected in the first and second biological samples. Alternatively, a polynucleotide encoding the selected peptide or polypeptide, or an APC expressing the selected peptide or polypeptide may be employed in place of the selected peptide or polypeptide itself. Within such methods, immunocomplexes between the selected peptide or polypeptide encoded by a polynucleotide, or expressed by the APC, and antibodies and/or antigen binding fragments in the biological sample are detected.

Methods in which T cell activation and/or the number of hematological malignancy polypepide-specific precursors are monitored may comprise the steps of: (a) incubating a first biological sample comprising CD4⁺ and/or CD8⁺ cells (e.g., bone marrow, peripheral blood or a fraction thereof), obtained from a patient prior to a therapy or immunization, with a hematological malignancy peptide or polypeptide, wherein the incubation is performed under conditions and for a time sufficient to allow specific activation, proliferation and/or lysis of T cells; (b) detecting an amount of activation, proliferation and/or lysis of the T cells; (c) repeating steps (a) and (b) using a second biological sample comprising CD4⁺ and/or CD8⁺ T cells, and taken from the same patient following therapy or immunization; and (d) comparing the amount of activation, proliferation and/or lysis of T cells in the first and second biological samples. Alternatively, a polynucleotide encoding a hematological malignancy related peptide, or an APC expressing such a peptide may be employed in place of the hematological malignancy peptide itself.

A biological sample for use within such methods may be any sample obtained from a patient that would be expected to contain antibodies, CD4⁺ T cells and/or CD8⁺ T cells. Suitable biological samples include blood, sera, ascites, bone marrow, pleural effusion and cerebrospinal fluid. A first biological sample may be obtained prior to initiation of therapy or immunization or part way through a therapy or vaccination regime. The second biological sample should be obtained in a similar manner, but at a time following additional therapy or immunization. The second biological sample may be obtained at the completion of, or part way through, therapy or immunization, provided that at least a portion of therapy or immunization takes place between the isolation of the first and second biological samples.

Incubation and detection steps for both samples may generally be performed as described above. A statistically significant increase in the number of immunocomplexes in the second sample relative to the first sample reflects successful therapy or immunization.

Administration of Pharmaceutical Compositions and Formulations

In certain embodiments, the present invention concerns formulation of one or more of the polynucleotide, polypeptide, peptide, antibody, or antigen binding fragment compositions disclosed herein in pharmaceutically acceptable solutions for administration to a cell or an animal, either alone, or in combination with one or more other modalities of anti-cancer therapy, or in combination with one or more diagnostic or therapeutic agents.

It will also be understood that, if desired, the nucleic acid segment, RNA, or DNA compositions disclosed herein may be administered in combination with other agents as well, such as, e.g., proteins or peptides or various pharmaceutically-active agents. As long as the composition comprises at least one of the genetic expression constructs disclosed herein, there is virtually no limit to other components that may also be included, given that the additional agents do not cause a significant adverse effect upon contact with the target cells or host tissues. The RNA- or DNA-derived compositions may thus be delivered along with various other agents as required in the particular instance. Such RNA or DNA compositions may be purified from host cells or other biological sources, or alternatively may be chemically synthesized as described herein. Likewise, such compositions may comprise substituted or derivatized RNA or DNA compositions. Such compositions may include one or more therapeutic gene constructs, either alone, or in combination with one or more modified peptide or nucleic acid substituent derivatives, and/or other anticancer therapeutics.

The formulation of pharmaceutically-acceptable excipients and carrier solutions are well-known to those of skill in the art, as is the development of suitable dosing and treatment regimens for using the particular compositions described herein in a variety of treatment regimens, including e.g., oral, intravenous, intranasal, transdermal, intraprostatic, intratumoral, and/or intramuscular administration and formulation.

Injectable Delivery

For example, the pharmaceutical compositions disclosed herein may be administered parenterally, intravenously, intramuscularly, or even intraperitoneally as described in U.S. Pat. Nos. 5,543,158, 5,641,515 and 5,399,363 (each specifically incorporated herein by reference in its entirety). Solutions of the active compounds as free-base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, Hoover, 1975). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions may be prepared by incorporating the gene therapy constructs in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Intranasal Delivery

One may use nasal solutions or sprays, aerosols or even inhalants for the treatment of hematological malignancies with one of more of the disclosed peptides and polynucleotides. Nasal solutions are usually aqueous solutions designed for administration to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of from about 5.5 to about 6.5. In addition, antimicrobial preservatives, similar to those used in ophthalmic preparations, and appropriate drug stabilizers, if required, may be included in the formulation. Various commercial nasal preparations are known.

Inhalations and inhalants are pharmaceutical preparations designed for delivering a drug or compound into the respiratory tree of a patient. A vapor or mist is administered and reaches the affected area, often to give relief from symptoms of bronchial and nasal congestion. However, this route can also be employed to deliver agents into the systemic circulation. Inhalations may be administered by the nasal or oral respiratory routes. The administration of inhalation solutions is only effective if the droplets are sufficiently fine and uniform in size so that the mist reaches the bronchioles.

Another group of products, also known as inhalations, and sometimes called insufflations, consists of finely powdered or liquid drugs that are carried into the respiratory passages by the use of special delivery systems, such as pharmaceutical aerosols, that hold a solution or suspension of the drug in a liquefied gas propellant. When released through a suitable valve and oral adapter, a metered does of the inhalation is propelled into the respiratory tract of the patient.

Particle size is of importance in the administration of this type of preparation. It has been reported that the optimum particle size for penetration into the pulmonary cavity is of the order of about 0.5 to about 7 μm. Fine mists are produced by pressurized aerosols and hence their use in considered advantageous.

Lipsome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the polynucleotide compositions of the present invention into suitable host cells. In particular, the polynucleotide compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-lives (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes have been used successfully with a number of cell types that are normally resistant to transfection by other procedures including T cell suspensions, primary hepatocyte cultures and PC12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1 990a; Imaizumi et al., 1 990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars, and drugs.

Alternatively, the invention provides for pharmaceutically acceptable nanocapsule formulations of the polynucleotide compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety). In particular, methods of polynucleotide polynucleotide delivery to a target cell using either nanoparticles or nanospheres (Schwab et al., 1994; Truong-Le et al., 1998) are also particularly contemplated to be useful in formulating the disclosed compositions for administration to an animal, and to a human in particular.

Therapeutic Agents and Kits

The invention also provides one or more of the hematological malignancy-related compositions formulated with one or more pharmaceutically acceptable excipients, carriers, diluents, adjuvants, and/or other components for use in the preaparation of medicaments, or diagnostic reagents, as well as various kits comprising one or more of such compositions, medicaments, or formulations intended for administration to an animal in need thereof, or for use in one or more diagnostic assays for identifying polynucleotides, polypeptides, and/or antibodies that are specific for one or more hematological malignancy-related compounds as described herein. In addition to the disclosed epitopes, antibodies and antigen binding fragments, antibody- or antigen binding fragment-encoding polynucleotides or additional anticancer agents, polynucleotides, peptides, antigens, or other therapeutic compounds as may be employed in the formulation of particular compositions and formulations disclosed herein, and particularly in the preparation of anticancer agents or anti-hematological malignancies therapies for administration to the affected mammal.

As such, preferred animals for administration of the pharmaceutical compositions disclosed herein include mammals, and particularly humans. Other preferred animals include primates, sheep, goats, bovines, equines, porcines, lupines, canines, and felines, as well as any other mammalian species commonly considered pets, livestock, or commercially relevant animal species. The compositions and formulations may include partially or significantly purified polypeptide, polynucleotide, or antibody or antigen binding fragment compositions, either alone, or in combination with one or more additional active ingredients, anticancer agents, vaccines, adjuvants, or other therapeutics which may be obtained from natural or recombinant sources, or which may be obtainable naturally or either chemically synthesized, or alternatively produced in vitro from recombinant host cells expressing one or more nucleic acid segments that encode one or more such additional active ingredients, carriers, adjuvants, cofactors, or other therapeutic compound.

Diagnostic Reagents and Kits

The invention further provides diagnostic reagents and kits comprising one or more such reagents for use in a variety of diagnostic assays, including for example, immunoassays such as ELISA and “sandwich”-type immunoassays. Such kits may preferably include at least a first peptide, or a first antibody or antigen binding fragment of the invention, a functional fragment thereof, or a cocktail thereof, and means for signal generation. The kit's components may be pre-attached to a solid support, or may be applied to the surface of a solid support when the kit is used. The signal generating means may come pre-associated with an antibody of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of microtiter plates, microspheres, or other materials suitable for immobilizing proteins, peptides, or polypeptides. Preferably, an enzyme that catalyzes the formation of a chemiluminescent or chromogenic product or the reduction of a chemiluminescent or chromogenic substrate is a component of the signal generating means. Such enzymes are well known in the art.

Such kits are useful in the detection, monitoring and diagnosis of conditions characterized by over-expression or inappropriate expression of hematological malignancy-related peptides, polypeptides, antibodies, and/or polynucleotides, as well as hybridomas, host cells, and vectors comprising one or more such compositions as disclosed herein.

The therapeutic and diagnostic kits of the present invention may also be prepared that comprise at least one of the antibody, peptide, antigen binding fragment, hybridoma, vector, vaccine, polynucleotide, or cellular compositions disclosed herein and instructions for using the composition as a diagnostic reagent or therapeutic agent. Containers for use in such kits may typically comprise at least one vial, test tube, flask, bottle, syringe or other suitable container, into which one or more of the diagnostic and/or therapeutic composition(s) may be placed, and preferably suitably aliquoted. Where a second therapeutic agent is also provided, the kit may also contain a second distinct container into which this second diagnostic and/or therapeutic composition may be placed. Alternatively, a plurality of compounds may be prepared in a single pharmaceutical composition, and may be packaged in a single container means, such as a vial, flask, syringe, bottle, or other suitable single container. The kits of the present invention will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the diagnostic or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

Polynucleotide Compositions

As used herein, the terms “DNA segment” and “polynucleotide” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the DNA segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

“Isolated,” as used herein, means that a polynucleotide is substantially away from other coding sequences, and that the DNA segment does not contain large portions of unrelated coding DNA, such as large chromosomal fragments or other functional genes or polypeptide coding regions. Of course, this refers to the DNA segment as originally isolated, and does not exclude genes or coding regions later added to the segment by the hand of man.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a hematological malignancy-related tumor protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native tumor protein. The effect on the immunogenicity of the encoded polypeptide may generally be assessed as described herein. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, Mo. (1978) A model of evolutionary change in proteins—Matrices for detecting distant relationships. In Dayhoff, Mo. (ed.) Atlas of Protein Sequence and Structure, National Biomedical Research Foundation, Washington DC. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Probes and Primers

In other embodiments of the present invention, the polynucleotide sequences provided herein can be advantageously used as probes or primers for nucleic acid hybridization. As such, it is contemplated that nucleic acid segments that comprise a sequence region of at least about 15, 20, 25, 30, 35, 40, 45, 50, 55, 60, 65, 70, 75, 80, 85, 90, or 95 nucleotide long contiguous sequence that has the same sequence as, or is complementary to, at least a 15, 20, 25, 30, 35, 40, 45, 50, 55, 60, 65, 70, 75, 80, 85, 90, or 95 nucleotide long contiguous sequence as disclosed in any one of SEQ ID NO:1 to SEQ ID NO:668 will find particular utility in a variety of hybridization embodiments. Longer contiguous identical or complementary sequences, e.g., those of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 525, 550, 575, 600, 650, 700, 750, 800, 850, 900, 950, or even 1000 or so nucleotides (including all intermediate lengths) and all full-length sequences as disclosed in SEQ ID NO:1 to SEQ ID NO:668 will also be of use in certain embodiments as probes, primers, or amplification targets and such like.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers, for use in preparing other genetic constructions, and for identifying and characterizing full-length polynucleotides and full, or substantially full-length cDNAs, mRNAs, and such like.

Polynucleotide molecules having sequence regions consisting of contiguous nucleotide stretches identical or complementary to one or more polynucleotide sequences as disclosed herein, are particularly contemplated as hybridization probes for use in, e.g., Southern hybridization analyses and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15, 20, 25, 30, 35, 40, 45, 50, 55, 60 or so and up to and including larger contiguous complementary sequences, including those of about 70, 80, 90, 100, 120, 140, 160, 180, or 200 or so nucleotides in length may also be used, according to the given desired goal, and the particular length of the complementary sequences one wishes to detect by hybridization analysis.

The use of a hybridization probe of about between about 20 and about 500 nucleotides in length allows the formation of a duplex molecule that is both stable and selective. Molecules having contiguous complementary sequences over stretches greater than about 20 or so bases in length are generally preferred, though, in order to increase stability and selectivity of the hybrid, and thereby improve the quality and degree of specific hybrid molecules obtained. One will generally prefer to design nucleic acid molecules having gene-complementary stretches of between about 25 and 300 or so contiguous nucleotides, or even longer where desired.

Hybridization probes may be selected from any portion of any of the sequences disclosed herein. All that is required is to review the sequence set forth in any one of SEQ ID NO:1 through SEQ ID NO:668, or to any contiguous portion of such a sequence, from about 15 to 30 nucleotides in length up to and including the full length sequences disclosed in any one of SEQ ID NO:1 through SEQ ID NO:668, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

Polynucleotide Identification and Characterization

Polynucleotides may be identified, prepared and/or manipulated using any of a variety of well established techniques. For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al., Proc. Natl. Acad. Sci. USA 93:10614-10619, 1996 and Heller et al., Proc. Natl. Acad. Sci USA 94:2150-2155, 1997). Alternatively, polynucleotides may be amplified from cDNA prepared from cells expressing the proteins described herein, such as hematological malignancy-related tumor cells. Such polynucleotides may be amplified via polymerase chain reaction (PCR). For this approach, sequence-specific primers may be designed based on the sequences provided herein, and may be purchased or synthesized.

An amplified portion of a polynucleotide of the present invention may be used to isolate a full length gene from a suitable library (e.g., a hematological malignancy-related tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software or algorithms or formulas well known in the art.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

Polynucleotide Expression in Host Cells

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, W H Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of beta-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.- cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells which contain and express a desired polynucleotide sequence may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Site-Specific Mutagenesis

Site-specific mutagenesis is a technique useful in the preparation of individual peptides, or biologically functional equivalent polypeptides, through specific mutagenesis of the underlying polynucleotides that encode them. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the antigenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

Polynucleotide Amplification Techniques

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qbeta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992, incorporated herein by reference in its entirety), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Methods based on ligation of two (or more) oligonucleotides in the presence of nucleic acid having the sequence of the resulting “di-oligonucleotide”, thereby amplifying the di-oligonucleotide (Wu and Dean, 1996, incorporated herein by reference in its entirety), may also be used in the amplification of DNA sequences of the present invention.

In vivo Polynucleotide Delivery Techniques

In additional embodiments, genetic constructs comprising one or more of the polynucleotides of the invention are introduced into cells in vivo. This may be achieved using any of a variety or well known approaches, several of which are outlined below for the purpose of illustration.

Adenovirus

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Adeno-Associated Viruses

AAV (Ridgeway, 1988; Hermonat and Muzycska, 1984) is a parovirus, discovered as a contamination of adenoviral stocks. It is a ubiquitous virus (antibodies are present in 85% of the US human population) that has not been linked to any disease. It is also classified as a dependovirus, because its replications is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka and McLaughlin, 1988).

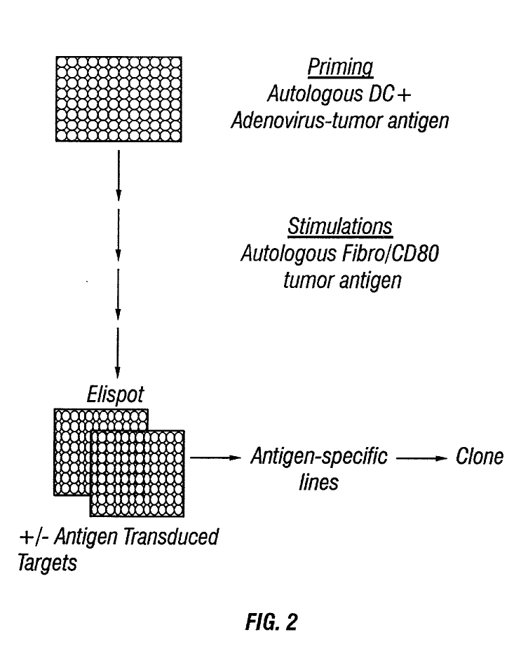

The AAV DNA is approximately 4.7 kilobases long. It contains two open reading frames and is flanked by two ITRs (FIG. 2). There are two major genes in the AAV genome: rep and cap. The rep gene codes for proteins responsible for viral replications, whereas cap codes for capsid protein VP 1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat and Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

Other Viral Vectors as Expression Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), lentiviruses, polio viruses and herpes viruses may be employed. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Non-viral Vectors

In order to effect expression of the oligonucleotide or polynucleotide sequences of the present invention, the expression construct must be delivered into a cell. This delivery may be accomplished in vitro, as in laboratory procedures for transforming cells lines, or in vivo or ex vivo, as in the treatment of certain disease states. As described above, one preferred mechanism for delivery is via viral infection where the expression construct is encapsulated in an infectious viral particle.

Once the expression construct has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments of the invention, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e. ex vivo treatment. Again, DNA encoding a particular gene may be delivered via this method and still be incorporated by the present invention.

Antisense Oligonucleotides

The end result of the flow of genetic information is the synthesis of protein. DNA is transcribed by polymerases into messenger RNA and translated on the ribosome to yield a folded, functional protein. Thus there are several steps along the route where protein synthesis can be inhibited. The native DNA segment coding for a polypeptide described herein, as all such mammalian DNA strands, has two strands: a sense strand and an antisense strand held together by hydrogen bonding. The messenger RNA coding for polypeptide has the same nucleotide sequence as the sense DNA strand except that the DNA thymidine is replaced by uridine. Thus, synthetic antisense nucleotide sequences will bind to a mRNA and inhibit expression of the protein encoded by that mRNA.

The targeting of antisense oligonucleotides to mRNA is thus one mechanism to shut down protein synthesis, and, consequently, represents a powerful and targeted therapeutic approach. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829, each specifically incorporated herein by reference in its entirety). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., 1988; Vasanthakumar and Ahmed, 1989; Peris et al., 1998; U.S. Pat. Nos. 5,801,154; 5,789,573; 5,718,709 and 5,610,288, each specifically incorporated herein by reference in its entirety). Antisense constructs have also been described that inhibit and can be used to treat a variety of abnormal cellular proliferations, e.g. cancer (U.S. Pat. Nos. 5,747,470; 5,591,317 and 5,783,683, each specifically incorporated herein by reference in its entirety).

Therefore, in exemplary embodiments, the invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein.

Selection of antisense compositions specific for a given gene sequence is based upon analysis of the chosen target sequence (i.e. in these illustrative examples the rat and human sequences) and determination of secondary structure, Tₘ, binding energy, relative stability, and antisense compositions were selected based upon their relative inability to form dimers, hairpins, or other secondary structures that would reduce or prohibit specific binding to the target mRNA in a host cell.

Highly preferred target regions of the mRNA, are those which are at or near the AUG translation initiation codon, and those sequences which were substantially complementary to 5′ regions of the mRNA. These secondary structure analyses and target site selection considerations were performed using v.4 of the OLIGO primer analysis software (Rychlik, 1997) and the BLASTN 2.0.5 algorithm software (Altschul et al., 1997).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., 1997). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane (Morris et al., 1997).

Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359 (specifically incorporated herein by reference). An example of the hepatitis 6 virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in (U.S. Pat. No. 4,987,071, specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In certain embodiments, it may be important to produce enzymatic cleaving agents which exhibit a high degree of specificity for the RNA of a desired target, such as one of the sequences disclosed herein. The enzymatic nucleic acid molecule is preferably targeted to a highly conserved sequence region of a target mRNA. Such enzymatic nucleic acid molecules can be delivered exogenously to specific cells as required. Alternatively, the ribozymes can be expressed from DNA or RNA vectors that are delivered to specific cells.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g., Usman and Cedergren, 1992). Ribozymes may be purified by gel electrophoresis using general methods or by high pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al., 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (e.g. Kashani-Saber et al., 1992; Ojwang et al., 1992; Chen et al., 1992; Yu et al., 1993; L'Huillier et al., 1992; Lisziewicz et al., 1993). Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

Ribozymes may be used as diagnostic tools to examine genetic drift and mutations within diseased cells. They can also be used to assess levels of the target RNA molecule. The close relationship between ribozyme activity and the structure of the target RNA allows the detection of mutations in any region of the molecule which alters the base-pairing and three-dimensional structure of the target RNA. By using multiple ribozymes, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These studies will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes are well known in the art, and include detection of the presence of mRNA associated with an IL-5 related condition. Such RNA is detected by determining the presence of a cleavage product after treatment with a ribozyme using standard methodology.

Peptide Nucleic Acids

In certain embodiments, the inventors contemplate the use of peptide nucleic acids (PNAs) in the practice of the methods of the invention. PNA is a DNA mimic in which the nucleobases are attached to a pseudopeptide backbone (Good and Nielsen, 1997). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (1997) and is incorporated herein by reference. As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., 1991; Hanvey et al., 1992; Hyrup and Nielsen, 1996; Neilsen, 1996). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc (Dueholm et al., 1994) or Fmoc (Thomson et al., 1995) protocols for solid-phase peptide synthesis, although other methods, including a modified Merrifield method, have been used (Christensen et al., 1995).

PNA monomers or ready-made oligomers are commercially available from PerSeptive Biosystems (Framingham, Mass.). PNA syntheses by either Boc or Fmoc protocols are straightforward using manual or automated protocols (Norton et al., 1995). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography (Norton et al., 1995) providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (Norton et al., 1995; Haaima et al., 1996; Stetsenko et al., 1996; Petersen et al., 1995; Ulmann et al., 1996; Koch et al., 1995; Orum et al., 1995; Footer et al., 1996; Griffith et al., 1995; Kremsky et al., 1996; Pardridge et al., 1995; Boffa et al., 1995; Landsdorp et al., 1996; Gambacorti-Passerini et al., 1996; Armitage et al., 1997; Seeger et al., 1997; Ruskowski et al., 1997). U.S. Pat. No. 5,700,922 discusses PNA-DNA-PNA chimeric molecules and their uses in diagnostics, modulating protein in organisms, and treatment of conditions susceptible to therapeutics.

In contrast to DNA and RNA, which contain negatively charged linkages, the PNA backbone is neutral. In spite of this dramatic alteration, PNAs recognize complementary DNA and RNA by Watson-Crick pairing (Egholm et al., 1993), validating the initial modeling by Nielsen et al. (1991). PNAs lack 3′ to 5′ polarity and can bind in either parallel or antiparallel fashion, with the antiparallel mode being preferred (Egholm et al., 1993).

Hybridization of DNA oligonucleotides to DNA and RNA is destabilized by electrostatic repulsion between the negatively charged phosphate backbones of the complementary strands. By contrast, the absence of charge repulsion in PNA-DNA or PNA-RNA duplexes increases the melting temperature (Tₘ) and reduces the dependence of Tₘ on the concentration of mono- or divalent cations (Nielsen et al., 1991). The enhanced rate and affinity of hybridization are significant because they are responsible for the surprising ability of PNAs to perform strand invasion of complementary sequences within relaxed double-stranded DNA. In addition, the efficient hybridization at inverted repeats suggests that PNAs can recognize secondary structure effectively within double-stranded DNA. Enhanced recognition also occurs with PNAs immobilized on surfaces, and Wang et al. have shown that support-bound PNAs can be used to detect hybridization events (Wang et al., 1996).

One might expect that tight binding of PNAs to complementary sequences would also increase binding to similar (but not identical) sequences, reducing the sequence specificity of PNA recognition. As with DNA hybridization, however, selective recognition can be achieved by balancing oligomer length and incubation temperature. Moreover, selective hybridization of PNAs is encouraged by PNA-DNA hybridization being less tolerant of base mismatches than DNA-DNA hybridization. For example, a single mismatch within a 16 bp PNA-DNA duplex can reduce the Tₘ by up to 15° C. (Egholm et al., 1993). This high level of discrimination has allowed the development of several PNA-based strategies for the analysis of point mutations (Wang et al., 1996; Carlsson et al., 1996; Thiede et al., 1996; Webb and Hurskainen, 1996; Perry-O'Keefe et al., 1996).

High-affinity binding provides clear advantages for molecular recognition and the development of new applications for PNAs. For example, 11-13 nucleotide PNAs inhibit the activity of telomerase, a ribonucleo-protein that extends telomere ends using an essential RNA template, while the analogous DNA oligomers do not (Norton et al., 1996).

Neutral PNAs are more hydrophobic than analogous DNA oligomers, and this can lead to difficulty solubilizing them at neutral pH, especially if the PNAs have a high purine content or if they have the potential to form secondary structures. Their solubility can be enhanced by attaching one or more positive charges to the PNA termini (Nielsen et al., 1991).

Findings by Allfrey and colleagues suggest that strand invasion will occur spontaneously at sequences within chromosomal DNA (Boffa et al., 1995; Boffa et al., 1996). These studies targeted PNAs to triplet repeats of the nucleotides CAG and used this recognition to purify transcriptionally active DNA (Boffa et al., 1995) and to inhibit transcription (Boffa et al., 1996). This result suggests that if PNAs can be delivered within cells then they will have the potential to be general sequence-specific regulators of gene expression. Studies and reviews concerning the use of PNAs as antisense and anti-gene agents include Nielsen et al. (1993b), Hanvey et al. (1992), and Good and Nielsen (1997). Koppelhus et al. (1997) have used PNAs to inhibit HIV-1 inverse transcription, showing that PNAs may be used for antiviral therapies.

Methods of characterizing the antisense binding properties of PNAs are discussed in Rose (1993) and Jensen et al. (1997). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs include use in DNA strand invasion (Nielsen et al., 1991), antisense inhibition (Hanvey et al., 1992), mutational analysis (Orum et al., 1993), enhancers of transcription (Mollegaard et al., 1994), nucleic acid purification (Orum et al., 1995), isolation of transcriptionally active genes (Boffa et al., 1995), blocking of transcription factor binding (Vickers et al., 1995), genome cleavage (Veselkov et al., 1996), biosensors (Wang et al., 1996), in situ hybridization (Thisted et al., 1996), and in a alternative to Southern blotting (Perry-O'Keefe, 1996).

Polypeptide, Peptides and Peptide Variants

The present invention, in other aspects, provides polypeptide compositions. Generally, a polypeptide of the invention will be an isolated polypeptide (or an epitope, variant, or active fragment thereof) derived from a mammalian species. Preferably, the polypeptide is encoded by a polynucleotide sequence disclosed herein or a sequence which hybridizes under moderately stringent conditions to a polynucleotide sequence disclosed herein. Alternatively, the polypeptide may be defined as a polypeptide which comprises a contiguous amino acid sequence from an amino acid sequence disclosed herein, or which polypeptide comprises an entire amino acid sequence disclosed herein.

In the present invention, a polypeptide composition is also understood to comprise one or more polypeptides that are immunologically reactive with antibodies generated against a polypeptide of the invention, particularly a polypeptide having the amino acid sequence encoded by the polynucleotides disclosed in SEQ ID NO:1-146, or to active fragments, or to variants or biological functional equivalents thereof.

Likewise, a polypeptide composition of the present invention is understood to comprise one or more polypeptides that are capable of eliciting antibodies that are immunologically reactive with one or more polypeptides encoded by one or more contiguous nucleic acid sequences contained in SEQ ID NO:1-146, or to active fragments, or to variants thereof, or to one or more nucleic acid sequences which hybridize to one or more of these sequences under conditions of moderate to high stringency. Particularly illustrative polypeptides include the amino acid sequences encoded by polynucleotides disclosed in SEQ ID NO:1-146.

As used herein, an active fragment of a polypeptide includes a whole or a portion of a polypeptide which is modified by conventional techniques, e.g., mutagenesis, or by addition, deletion, or substitution, but which active fragment exhibits substantially the same structure function, antigenicity, etc., as a polypeptide as described herein.

In certain illustrative embodiments, the polypeptides of the invention will comprise at least an immunogenic portion of a hematological malignancy-related tumor protein or a variant thereof, as described herein. As noted above, a “hematological malignancy-related tumor protein” is a protein that is expressed by hematological malignancy-related tumor cells. Proteins that are hematological malignancy-related tumor proteins also react detectably within an immunoassay (such as an ELISA) with antisera from a patient with hematological malignancy. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of a hematological malignancy-related tumor protein or a variant thereof. Certain preferred immunogenic portions include peptides in which an N-terminal leader sequence and/or transmembrane domain have been deleted. Other preferred immunogenic portions may contain a small N- and/or C-terminal deletion (e.g., 1-30 amino acids, preferably 5-15 amino acids), relative to the mature protein.

Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a native hematological malignancy-related tumor protein is a portion that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As noted above, a composition may comprise a variant of a native hematological malignancy-related tumor protein. A polypeptide “variant,” as used herein, is a polypeptide that differs from a native hematological malignancy-related tumor protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants encompassed by the present invention include those exhibiting at least about 70%, 75%, 80%, 85%, 90%, 91%, 92%, 93%, 94%, 95%, 96%, 97%, 98%, or 99% or more identity (determined as described above) to the polypeptides disclosed herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided. Such proteins comprise a polypeptide as described herein together with an unrelated immunogenic protein. Preferably the immunogenic protein is capable of eliciting a recall response. Examples of such proteins include tetanus, tuberculosis and hepatitis proteins (see, for example, Stoute et al. New Engl. J. Med., 336:86-91, 1997).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Binding Agents

The present invention further employs agents, such as antibodies and antigen-binding fragments thereof, that specifically bind to a hematological malignancy-related antigen. As used herein, an antibody, or antigen-binding fragment thereof, is said to “specifically bind” to a hematological malignancy-related antigen if it reacts at a detectable level (within, for example, an ELISA) with, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant maybe determined using methods well known in the art.

Binding agents may be further capable of differentiating between patients with and without a hematological malignancy. Such binding agents generate a signal indicating the presence of a hematological malignancy in at least about 20% of patients with the disease, and will generate a negative signal indicating the absence of the disease in at least about 90% of individuals without the disease. To determine whether a binding agent satisfies this requirement, biological samples (e.g., blood, sera, urine and/or tumor biopsies) from patients with and without a hematological malignancy (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies, and fragments thereof, of the present invention may be coupled to one or more therapeutic agents, such as radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein. For certain in vivo and ex vivo therapies, an antibody or fragment thereof is preferably coupled to a cytotoxic agent, such as a radioactive or chemotherapeutic moiety.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234), peptides and polysaccharides such as aminodextran (e.g., U.S. Pat. No. 4,699,784). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562 discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

Vaccines

In certain preferred embodiments of the present invention, vaccines are provided. The vaccines will generally comprise one or more pharmaceutical compositions, such as those discussed above, in combination with an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

Illustrative vaccines may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB2,200,651; EP0,345,242; WO91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Uhner et al., Science 259:1745-1749, 1993 and reviewed by Cohen, Science 259:1691-1692, 1993. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD 11) and costimulatory molecules (e.g., CD40, CD80, CD86 and 4-1BB).

APCs may generally be transfected with a polynucleotide encoding a hematological malignancy-related tumor protein (or portion or other variant thereof) such that the hematological malignancy-related tumor polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the hematological malignancy-related tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Cancer Therapy

In further aspects of the present invention, the compositions described herein may be used for immunotherapy of cancer, such as hematological malignancy. Within such methods, pharmaceutical compositions and vaccines are typically administered to a patient. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. A cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs. Administration may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a hematological malignancy-related tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnosis

In general, a cancer may be detected in a patient based on the presence of one or more hematological malignancy-related tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as hematological malignancy. In addition, such proteins may be useful for the detection of other cancers. The binding agents provided herein generally permit detection of the level of antigen that binds to the agent in the biological sample. Polynucleotide primers and probes may be used to detect the level of mRNA encoding a tumor protein, which is also indicative of the presence or absence of a cancer. In general, a hematological malignancy-related tumor sequence should be present at a level that is at least three fold higher in tumor tissue than in normal tissue

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length hematological malignancy-related tumor proteins and portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with hematological malignancy. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as hematological malignancy, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al, Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use hematological malignancy-related tumor polypeptides to detect antibodies that bind to such polypeptides in a biological sample. The detection of such hematological malignancy-related tumor protein specific antibodies may correlate with the presence of a cancer.

A cancer may also, or alternatively, be detected based on the presence of T cells that specifically react with a hematological malignancy-related tumor protein in a biological sample. Within certain methods, a biological sample comprising CD4⁺ and/or CD8⁺ T cells isolated from a patient is incubated with a hematological malignancy-related tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of hematological malignancy-related tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a hematological malignancy-related tumor protein in a biological sample. For example, at least two oligonucleotide primers may be employed in a polymerase chain reaction (PCR) based assay to amplify a portion of a hematological malignancy-related tumor cDNA derived from a biological sample, wherein at least one of the oligonucleotide primers is specific for (i.e., hybridizes to) a polynucleotide encoding the hematological malignancy-related tumor protein. The amplified cDNA is then separated and detected using techniques well known in the art, such as gel electrophoresis. Similarly, oligonucleotide probes that specifically hybridize to a polynucleotide encoding a hematological malignancy-related tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a hematological malignancy-related tumor protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least contiguous nucleotides, more preferably at least 15 contiguous nucleotides, of a DNA molecule having a sequence recited in SEQ ID NO:1-146. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple hematological malignancy-related tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Preparation of DNA Sequences

Certain nucleic acid sequences of cDNA molecules encoding portions of hematological malignancy-related antigens were isolated by PCR™-based subtraction. This technique serves to normalize differentially expressed cDNAs, facilitating the recovery of rare transcripts, and also has the advantage of permitting enrichment of cDNAs with small amounts of polyA RNA material and without multiple rounds of hybridization. To obtain antigens overexpressed in non-Hodgkin's lymphomas, two subtractions were performed with a tester library prepared from a pool of three T cell non-Hodgkin's lymphoma mRNAs. The two libraries were independently subtracted with different pools of driver cDNAs. Driver #1 contained cDNA prepared from specific normal tissues (lymph node, bone marrow, T cells, heart and brain), and this subtraction generated the library TCS-D1 (T cell non-Hodgkin's lymphoma subtracted library with driver #1). Driver #2 contained non-specific normal tissues (colon, large intestine, lung, pancreas, spinal cord, skeletal muscle, liver, kidney, skin and brain), and this subtraction generated the library TCS-D2 (T cell non-Hodgkin's lymphoma subtraction library with driver #2). Two other subtractions were performed with a tester library prepared from a pool of three B cell non-Hodgkin's lymphoma mRNAs. The two libraries were independently subtracted with different pools of driver cDNAs. Driver #1 contained cDNA prepared from specific normal tissues (lymph node, bone marrow, B cells, heart and brain), and this subtraction generated the library BCNHL/D1 (B cell non-Hodgkin's lymphoma subtracted library with driver #1). Driver #2 contained non-specific normal tissues (brain, lung, pancreas, spinal cord, skeletal muscle, colon, spleen, large intestine and PBMC), and this subtraction generated the library BCNHL/D2 (B cell non-Hodgkin's lymphoma subtraction library with driver #2). PCR™-amplified pools were generated from the subtracted libraries and clones were sequenced.

Hematological malignancy-related antigen sequences may be further characterized using any of a variety of well known techniques. For example, PCR™ amplified clones may be arrayed onto glass slides for microarray analysis. To determine tissue distribution, the arrayed clones may be used as targets to be hybridized with different first strand cDNA probes, including lymphoma probes, leukemia probes and probes from different normal tissues. Leukemia and lymphoma probes may be generated from cryopreserved samples obtained at the time of diagnosis from NHL, Hodgkin's disease, AML, CML, CLL, ALL, MDS and myeloma patients with poor outcome (patients who failed to achieve complete remission following conventional chemotherapy or relapsed) or good outcome (patients who achieved long term remission). To analyze gene expression during hematopoetic differentiation, probes may be generated from >95% pure fractions of CD34+, CD2+, CD14+, CD15+ and CD19+ cells derived from healthy individuals.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo (e.g., by transfecting antigen-presenting cells, such as dendritic cells, with a cDNA construct encoding a hematological malignancy-related antigen, and administering the transfected cells to the patient).

A portion of a sequence complementary to a coding sequence (i.e., an antisense polynucleotide) may also be used as a probe or to modulate hematological malignancy-related antigen expression. cDNA constructs that can be transcribed into antisense RNA may also be introduced into cells or tissues to facilitate the production of antisense RNA. An antisense polynucleotide may be used, as described herein, to inhibit expression of a hematological malignancy-related antigen. Antisense technology can be used to control gene expression through triple-helix formation, which compromises the ability of the double helix to open sufficiently for the binding of polymerases, transcription factors or regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a gene (e.g., promoter, enhancer or transcription initiation site), and block transcription of the gene; or to block translation by inhibiting binding of a transcript to ribosomes.

A portion of a coding sequence or of a complementary sequence may also be designed as a probe or primer to detect gene expression. Probes may be labeled with a variety of reporter groups, such as radionuclides and enzymes, and are preferably at least 10 nucleotides in length, more preferably at least 20 nucleotides in length and still more preferably at least 30 nucleotides in length. Primers, as noted above, are preferably 22-30 nucleotides in length.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Hematological malignancy-related antigen polynucleotides may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Therapeutic Methods

In further aspects of the present invention, the compositions described herein may be used for immunotherapy of hematological malignancies including adult and pediatric AML, CML, ALL, CLL, myelodysplastic syndromes (MDS), myeloproliferative syndromes (MPS), secondary leukemia, multiple myeloma, Hodgkin's lymphoma and Non-Hodgkin's lymphomas. In addition, compositions described herein may be used for therapy of diseases associated with an autoimmune response against hematopoetic precursor cells, such as severe aplastic anemia.

Immunotherapy may be performed using any of a variety of techniques, in which compounds or cells provided herein function to remove hematological malignancy-related antigen-expressing cells from a patient. Such removal may take place as a result of enhancing or inducing an immune response in a patient specific for hematological malignancy-related antigen or a cell expressing hematological malignancy-related antigen. Alternatively, hematological malignancy-related antigen-expressing cells may be removed ex vivo (e.g., by treatment of autologous bone marrow, peripheral blood or a fraction of bone marrow or peripheral blood). Fractions of bone marrow or peripheral blood may be obtained using any standard technique in the art.

Within such methods, pharmaceutical compositions and vaccines are typically administered to a patient. As used herein, a “patient” refers to any warm-blooded animal, preferably a human. A patient may or may not be afflicted with a hematological malignancy. Accordingly, the above pharmaceutical compositions and vaccines may be used to prevent the development of a malignancy or to treat a patient afflicted with a malignancy. A hematological malignancy may be diagnosed using criteria generally accepted in the art. Pharmaceutical compositions and vaccines may be administered either prior to or following surgical removal of primary tumors and/or treatment such as administration of radiotherapy or conventional chemotherapeutic drugs, or bone marrow transplantation (autologous, allogeneic or syngeneic).

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

The compositions provided herein may be used alone or in combination with conventional therapeutic regimens such as surgery, irradiation, chemotherapy and/or bone marrow transplantation (autologous, syngeneic, allogeneic or unrelated). As discussed in greater detail below, binding agents and T cells as provided herein may be used for purging of autologous stem cells. Such purging may be beneficial prior to, for example, bone marrow transplantation or transfusion of blood or components thereof. Binding agents, T cells, antigen presenting cells (APC) and compositions provided herein may further be used for expanding and stimulating (or priming) autologous, allogeneic, syngeneic or unrelated hematological malignancy-related antigen-specific T-cells in vitro and/or in vivo. Such hematological malignancy-related antigen-specific T cells may be used, for example, within donor lymphocyte infusions.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 100 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a hematological malignancy-related antigen generally correlate with an improved clinical outcome. Such immune responses may generally be evaluated using standard proliferation, cytotoxicity or cytokine assays, which may be performed using samples obtained from a patient before and after treatment.

Within further aspects, methods for inhibiting the development of a malignant disease associated with hematological malignancy-related antigen expression involve the administration of autologous T cells that have been activated in response to a hematological malignancy-related antigen polypeptide or hematological malignancy-related antigen-expressing APC, as described above. Such T cells may be CD4⁺ and/or CD8⁺, and may be proliferated as described above. The T cells may be administered to the individual in an amount effective to inhibit the development of a malignant disease. Typically, about 1×10⁹ to 1×10¹¹ T cells/M² are administered intravenously, intracavitary or in the bed of a resected tumor. It will be evident to those skilled in the art that the number of cells and the frequency of administration will be dependent upon the response of the patient.

Within certain embodiments, T cells may be stimulated prior to an autologous bone marrow transplantation. Such stimulation may take place in vivo or in vitro. For in vitro stimulation, bone marrow and/or peripheral blood (or a fraction of bone marrow or peripheral blood) obtained from a patient may be contacted with a hematological malignancy-related antigen polypeptide, a polynucleotide encoding a hematological malignancy-related antigen polypeptide and/or an APC that expresses a hematological malignancy-related antigen polypeptide under conditions and for a time sufficient to permit the stimulation of T cells as described above. Bone marrow, peripheral blood stem cells and/or hematological malignancy-related antigen-specific T cells may then be administered to a patient using standard techniques.

Within related embodiments, T cells of a related or unrelated donor may be stimulated prior to a syngeneic or allogeneic (related or unrelated) bone marrow transplantation. Such stimulation may take place in vivo or in vitro. For in vitro stimulation, bone marrow and/or peripheral blood (or a fraction of bone marrow or peripheral blood) obtained from a related or unrelated donor may be contacted with a hematological malignancy-related antigen polypeptide, hematological malignancy-related antigen polynucleotide and/or APC that expresses a hematological malignancy-related antigen polypeptide under conditions and for a time sufficient to permit the stimulation of T cells as described above. Bone marrow, peripheral blood stem cells and/or hematological malignancy-related antigen-specific T cells may then be administered to a patient using standard techniques.

Within other embodiments, hematological malignancy-related antigen-specific T cells, antibodies or antigen-binding fragments thereof as described herein may be used to remove cells expressing hematological malignancy-related antigen from a biological sample, such as autologous bone marrow, peripheral blood or a fraction of bone marrow or peripheral blood (e.g., CD34⁺ enriched peripheral blood (PB) prior to administration to a patient). Such methods may be performed by contacting the biological sample with such T cells, antibodies or antibody fragments under conditions and for a time sufficient to permit the reduction of hematological malignancy-related antigen expressing cells to less than 10%, preferably less than 5% and more preferably less than 1%, of the total number of myeloid or lymphatic cells in the bone marrow or peripheral blood. Such contact may be achieved, for example, using a column to which antibodies are attached using standard techniques. Antigen-expressing cells are retained on the column. The extent to which such cells have been removed may be readily determined by standard methods such as, for example, qualitative and quantitative PCR analysis, morphology, immunohistochemistry and FACS analysis. Bone marrow or PB (or a fraction thereof) may then be administered to a patient using standard techniques.

Diagnostic Methods

In general, a hematological malignancy may be detected in a patient based on the presence of hematological malignancy-related antigen and/or polynucleotide in a biological sample (such as blood, sera, urine and/or tumor biopsies) obtained from the patient. In other words, hematological malignancy-related antigens may be used as a marker to indicate the presence or absence of such a malignancy. The binding agents provided herein generally permit detection of the level of antigen that binds to the agent in the biological sample. Polynucleotide primers and probes may be used to detect the level of mRNA encoding hematological malignancy-related antigen, which is also indicative of the presence or absence of a hematological malignancy. In general, hematological malignancy-related antigen should be present at a level that is at least three fold higher in a sample obtained from a patient afflicted with a hematological malignancy than in the sample obtained from an individual not so afflicted.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a hematological malignancy in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length hematological malignancy-related antigens and portions thereof to which the binding agent binds, as described above.

The solid support may be any material known to those of ordinary skill in the art to which the hematological malignancy-related antigen polypeptide may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with a hematological malignancy. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a hematological malignancy, the signal detected from the reporter group that remains bound to the solid support is generally compared to a signal that corresponds to a predetermined cut-off value. In one preferred embodiment, the cut-off value for the detection of a hematological malignancy is the average mean signal obtained when the immobilized antibody is incubated with samples from patients without the malignancy. In general, a sample generating a signal that is three standard deviations above the predetermined cut-off value is considered positive for the malignancy. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a malignancy.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a hematological malignancy. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the hematological malignancy-related antigen sequences or binding agents of the present invention. The above descriptions are intended to be exemplary only. For example, it will be apparent to those of ordinary skill in the art that the above protocols may be readily modified to use hematological malignancy-related antigen polypeptides to detect antibodies that bind to such polypeptides in a biological sample. The detection of hematological malignancy-related antigen-specific antibodies may correlate with the presence of a hematological.

A malignancy may also, or alternatively, be detected based on the presence of T cells that specifically react with hematological malignancy-related antigen in a biological sample. Within certain methods, a biological sample comprising CD4⁺ and/or CD8⁺ T cells isolated from a patient is incubated with a hematological malignancy-related antigen polypeptide, a polynucleotide encoding such a polypeptide and/or an APC that expresses such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with Mtb-81 or Mtb-67.2 polypeptide (e.g., 5-25 μg/ml). It may be desirable to incubate another aliquot of a T cell sample in the absence of hematological malignancy-related antigen polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a hematological malignancy in the patient.

As noted above, a hematological malignancy may also, or alternatively, be detected based on the level of mRNA encoding hematological malignancy-related antigen in a biological sample. For example, at least two oligonucleotide primers may be employed in a polymerase chain reaction (PCR) based assay to amplify a portion of hematological malignancy-related antigen cDNA derived from a biological sample, wherein at least one of the oligonucleotide primers is specific for (i.e., hybridizes to) a polynucleotide encoding the hematological malignancy-related antigen protein. The amplified cDNA is then separated and detected using techniques well known in the art, such as gel electrophoresis. Similarly, oligonucleotide probes that specifically hybridize to a polynucleotide encoding hematological malignancy-related antigen may be used in a hybridization assay to detect the presence of polynucleotide encoding hematological malignancy-related antigen in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding hematological malignancy-related antigen that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample such as a biopsy tissue and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a hematological malignancy. The amplification reaction may be performed on several dilutions of cDNA spanning two orders of magnitude. A two-fold or greater increase in expression in several dilutions of the test patient sample as compared to the same dilutions of the sample from a normal individual is typically considered positive.

In preferred embodiments, such assays may be performed using samples enriched for cells expressing the hematological malignancy-related antigen(s) of interest. Such enrichment may be achieved, for example, using a binding agent as provided herein to remove the cells from the remainder of the biological sample. The removed cells may then be assayed as described above for biological samples.

In further embodiments, hematological malignancy-related antigens may be used as markers for monitoring disease progression or the response to therapy of a hematological malignancy. In this embodiment, assays as described above for the diagnosis of a hematological malignancy may be performed over time, and the change in the level of reactive polypeptide(s) evaluated. For example, the assays may be performed every 24-72 hours for a period of 6 months to 1 year, and thereafter performed as needed. In general, a malignancy is progressing in those patients in whom the level of polypeptide detected by the binding agent increases over time. In contrast, the malignancy is not progressing when the level of reactive polypeptide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple markers may be assayed within a given sample. It will be apparent that binding agents specific for different proteins provided herein may be combined within a single assay. Further, multiple primers or probes may be used concurrently. The selection of markers may be based on routine experiments to determine combinations that results in optimal sensitivity.

Further diagnostic applications include the detection of extramedullary disease (e.g., cerebral infiltration of blasts in leukemias). Within such methods, a binding agent may be coupled to a tracer substance, and the diagnosis is performed in vivo using well known techniques. Coupled binding agent may be administered as described above, and extramedullary disease may be detected based on assaying the presence of tracer substance. Alternatively, a tracer substance may be associated with a T cell specific for hematological malignancy-related antigen, permitting detection of extramedullary disease based on assays to detect the location of the tracer substance.

Exemplary Definitions

In accordance with the present invention, nucleic acid sequences include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from native sources, chemically synthesized, modified, or otherwise prepared in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription.

Structural gene: A gene or sequence region that is expressed to produce an encoded peptide or polypeptide.

Transformation: A process of introducing an exogenous polynucleotide sequence (e.g., a vector, a recombinant DNA or RNA molecule) into a host cell or protoplast in which that exogenous nucleic acid segment is incorporated into at least a first chromosome or is capable of autonomous replication within the transformed host cell. Transfection, electroporation, and naked nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Transgenic animal: An animal or a progeny or an offspring of any generation thereof that is derived from a transformed animal cell, wherein the animal's DNA contains an introduced exogenous nucleic acid molecule not originally present in a native, wild type, non-transgenic animal of the same species. The terms “transgenic animal” and “transformed animal” have sometimes been used in the art as synonymous terms to define an animal, the genetic contents of which has been modified to contain one or more exogenous nucleic acid segments.

Vector: A nucleic acid molecule, typically comprised of DNA, capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

As noted above, the present invention is generally directed to compositions and methods for using the compositions, for example in the therapy and diagnosis of cancer, such as hematological malignancy. Certain illustrative compositions described herein include hematological malignancy-related tumor polypeptides, polynucleotides encoding such polypeptides, binding agents such as antibodies, antigen presenting cells (APCs) and/or immune system cells (e.g., T cells). A “hematological malignancy-related tumor protein,” as the term is used herein, refers generally to a protein that is expressed in hematological malignancy-related tumor cells at a level that is at least two fold, and preferably at least five fold, greater than the level of expression in a normal tissue, as determined using a representative assay provided herein. Certain hematological malignancy-related tumor proteins are tumor proteins that react detectably (within an immunoassay, such as an ELISA or Western blot) with antisera of a patient afflicted with hematological malignancy.

Biological Functional Equivalents

Modification and changes may be made in the structure of the polynucleotides and peptides of the present invention and still obtain a functional molecule that encodes a peptide with desirable characteristics, or still obtain a genetic construct with the desirable expression specificity and/or properties. As it is often desirable to introduce one or more mutations into a specific polynucleotide sequence, various means of introducing mutations into a polynucleotide or peptide sequence known to those of skill in the art may be employed for the preparation of heterologous sequences that may be introduced into the selected cell or animal species. In certain circumstances, the resulting encoded peptide sequence is altered by this mutation, or in other cases, the sequence of the peptide is unchanged by one or more mutations in the encoding polynucleotide. In other circumstances, one or more changes are introduced into the promoter and/or enhancer regions of the polynucleotide constructs to alter the activity, or specificity of the expression elements and thus alter the expression of the heterologous therapeutic nucleic acid segment operably positioned under the control of the elements.

When it is desirable to alter the amino acid sequence of one or more of the heterologous peptides encoded by the expression construct to create an equivalent, or even an improved, second-generation molecules, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those that are within ±1 are particularly preferred, and those within ±0.5 are even more particularly preferred. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine.

### EXAMPLES

The following examples are included to demonstrate preferred embodiments of the invention. However, those of skill in the art should, in light of the present disclosure, appreciate that many changes can be made in the specific embodiments which are disclosed and still obtain a like or similar result without departing from the spirit and scope of the invention described in the appended claims.

### Example 1

### Identification of Hematological Malignancy-Related Antigen Polynucleotides

This Example illustrates the identification of hematological malignancy-related antigen polynucleotides from non-Hodgkin's lymphomas.

Hematological malignancy-related antigen polynucleotides were isolated by PCR-based subtraction. PolyA mRNA was prepared from T cell non-Hodgkin's lymphomas, B cell non-Hodgkin's lymphomas and normal tissues. Six cDNA libraries were constructed, PCR-subtracted and analyzed. Two libraries were constructed using pools of three T cell non-Hodgkin's lymphoma mRNAs (referred to herein as TCS libraries). Two others were constructed using pools of three B cell non-Hodgkin's lymphoma mRNAs (referred to herein as BCNHL libraries). Two other libraries were constructed using a pool of 2 Hodgkin's lymphoma mRNAs (referred to herein as HLS libraries. cDNA synthesis, hybridization and PCR amplification were performed according to Clontech's user manual (PCR-Select cDNA Subtraction), with the following changes: 1) cDNA was restricted with a mixture of enzymes, including MscI, PvuII, StuI and DraI, instead of the single enzyme RsaI; and 2) the ratio of driver to tester cDNA was increased in the hybridization steps (to 76:1) to give a more stringent subtraction.

The two TCS libraries were independently subtracted with different pools of driver cDNAs. Driver #1 contained cDNA prepared from specific normal tissues (lymph node, bone marrow, T cells, heart and brain), and this subtraction generated the library TCS-D1 (T cell non-Hodgkin's lymphoma subtracted library with driver #1). Driver #2 contained non-specific normal tissues (colon, large intestine, lung, pancreas, spinal cord, skeletal muscle, liver, kidney, skin and brain), and this subtraction generated the library TCS-D2 (T cell non-Hodgkin's lymphoma subtraction library with driver #2).

Similarly, the two BCNHL libraries were independently subtracted with different pools of driver cDNAs. Driver #1 contained cDNA prepared from specific normal tissues (lymph node, bone marrow, B cells, heart and brain), and this subtraction generated the library BCNHL/D1 (B cell non-Hodgkin's lymphoma subtracted library with driver #1). Driver #2 contained non-specific normal tissues (brain, lung, pancreas, spinal cord, skeletal muscle, colon, spleen, large intestine and PBMC), and this subtraction generated the library BCNHL/D2 (B cell non-Hodgkin's lymphoma subtraction library with driver #2).

The two HLS libraries were independently subtracted with different pools of driver cDNAs. Driver #1 contained cDNA prepared from specific normal tissues (lymph node, bone marrow, B cells and lung) and this subtraction generated HLS-D1 (Hodgkin's lymphoma subtraction library with driver #1). Driver #2 contained non-specific normal tissues (colon, large intestine, lung, pancreas, spinal cord, skeletal muscle, liver, kidney, skin and brain) and this generated the library HLS-D2 (Hodgkin's lymphoma subtraction library with driver #2).

To analyze the efficiency of the subtraction, actin (a housekeeping gene) was PCR amplified from dilutions of subtracted as well as unsubtracted PCR samples. Furthermore, the complexity and redundancy of each library was characterized by sequencing 96 clones from each of the PCR subtraction libraries (TCS-D1, TCS-D2, BCNHL/D1, BCNHL/D2, HLS-D1 and HLS-D2). These analyses indicated that the libraries are enriched for genes overexpressed in leukemia tissues and specifically T cell and B cell non-Hodgkin's lymphoma and M. Hodgkin's lymphoma samples.

Following PCR amplification, the cDNAs were cloned into the pCR2.1-TOPO plasmid vector (Invitrogen).

Sequences obtained from these analyses were searched against known sequences in the publicly available databases using the BLAST 2.0 release. The default BLAST parameters used were as follows: GAP PARAMETERS: Open Gap=0, Extended Gap=0; OUTPUT PARAMETERS: Expect=10.0, Threshold=0, Number of Alignments=250; For BLASTN, the search parameters were as follows: Mismatch=−3, Reward=1, Word size=0. The alignments were presented pair-wise, with a window percent identity=22. All available protein and nucleotide databases were searched, including, PIR, SwissPROT, GenBank, Mouse EST, Human EST, Other EST, Human repeat and high throughput sequences, and published patents and patent application database.

From these, a number of unique sequences were identified that represented novel polynucleotide sequences that had not previously been described in the GenBank and other sequence databases. A number of other sequences were identified that appeared to contain significant homology with one or more sequences previously identified in the databases, although they were described only as genomic or cDNA clones, and had no known function. The remaining sequences corresponded to known genes. The clones obtained from this analysis are summarized in Tables 2-5.

TABLE 2
T CELL NON-HODGKIN'S LYMPHOMA SUBTRACTED PCR ™
LIBRARY - SPECIFIC TISSUE DRIVER

| Clone No. | Comments |
|---|---|
| TCD1_F1 | Previously Unknown |
| TCD1_C2 | Previously Unknown |
| TCD1_D6 | Previously Unknown |
| TCD1_F8 | Previously Unknown |
| TCD1_G8 | Previously Unknown |
| TCD1_H12 | Previously Unknown |
| TCD1_B12 | Previously Unknown |
| TCD1_F12 | Previously Unknown |
| TCD1_H5 | Previously Unknown |
| TCD1_A6 | Previously Unknown |
| TCD1_B1 | Previously Unknown |
| TCD1_E1 | Previously Unknown |
| TCD1_D2 | Previously Unknown |
| TCD1_H2 | Previously Unknown |
| TCD1_C4 | Previously Unknown |
| TCD1_F5 | Previously Unknown |
| TCD1_C6 | Previously Unknown |
| TCD1_A7 | Previously Unknown |
| TCD1_B7 | Previously Unknown |
| TCD1_F7 | Previously Unknown |
| TCD1_A8 | Previously Unknown |
| TCD1_D8 | Previously Unknown |
| TCD1_E8 | Previously Unknown |
| TCD1_H9 | Previously Unknown |
| TCD1_C10 | Previously Unknown |
| TCD1_G11 | Previously Unknown |
| TCD1_A12 | Previously Unknown |
| TCD1_D12 | Previously Unknown |
| TCD1_G6 | H. sapiens mRNA; cDNA DKFZp566A201 |
| TCD1_C11 | H. sapiens mRNA; cDNA DKFZp566A201 |
| TCD1_F2 | H. sapiens chromosome 11 from 11p15.5 region |
| TCD1_G12 | H. sapiens chromosome 11 from 11p15.5 region |
| TCD1_D4 | H. sapiens mRNA for T cell leukemia/lymphoma 1 |
| TCD1_B6 | H. sapiens mRNA for T cell leukemia/lymphoma 1 |
| TCD1_A2 | Human chromosome 14 DNA sequence |
| TCD1_B2 | H. sapiens clone 25226 mLRNA sequence |
| TCD1_E3 | Human DNA sequence from clone 686C3 on chr. 20 |
| TCD1_C5 | H. sapiens upregulated by 1,25-dihydroxyvitamin D-3 |
| | (VDUP1) |
| TCD1_D5 | H. sapiens DNA sequence from PAC 63G5 on chr. |
| | 22q12.3-13.1 |
| TCD1_H6 | H. sapiens chr. 17, clone hRPK.318_A_15 |
| TCD1_G7 | Genomic sequence from human 9q34 |
| TCD1_D9 | Human mRNA for KIAA0386 gene |
| TCD1_E9 | H. sapiens DNA sequence from PAC 434O14 on chr. |
| | 1q32.3.-41 |
| TCD1_E11 | H. sapiens chr. 22q12 BAC clone bk256d12 in MDR region |
| TCD1_E12 | H. sapiens mRNA for KIAA1055 protein |
| TCD1_G3 | H. sapiens tumor necrosis factor receptor superfamily |
| | member 8 (TNFRSF8) |
| TCD1_B8 | H. sapiens tumor necrosis factor receptor superfamily |
| | member 8 (TNFRSF8) |
| TCD1_A1 | H. sapiens mRNA for GS3955 (putative serine/threonine |
| | kinase) |
| TCD1_C1 | H. sapiens mRNA for IRC1 protein |
| TCD1_D1 | H. sapiens nucleolar phosphoprotein p130 |
| TCD1_G1 | H. sapiens splicing factor (45kD) (SPF45) |
| TCD1_E2 | H. sapiens cAMP phosphodiesterase PDE7 (PDE7A1) |
| TCD1_A3 | H. sapiens CDC13 (cell division cycle 16, S. cerevisiae, |
| | homolog) |
| TCD1_B3 | H. sapiens cyclin Cd |
| TCD1_A4 | H. sapiens retinoblastoma-like 2 (P130) (RBL2) |
| TCD1_B5 | Human lymphocyte associated receptor of death 8 mRNA, |
| | altern. splice |
| TCD1_G5 | H. sapiens clathrin, heavy polypeptide-like 2 (CLTCL2) |
| TCD1_F6 | Human tumor necrosis factor type 1 receptor assoc. protein |
| | (TRAP1) |
| TCD1_C7 | H. sapiens phospholipase C, beta 2 (PLCB2) |
| TCD1_D7 | H. sapiens NADH: ubiquinone dehydrogenase 51 kDa |
| | subunit (NDUFV1) |
| TCD1_E7 | H. sapiens T-cell gamma receptor locus |
| TCD1_H8 | Rbr-2 = retinoblastoma susceptibility gene |
| TCD1_B9 | H. sapiens mRNA for eukaryotic initiation factor 4All |
| TCD1_C9 | H. sapiens asparaginyl-tRNA synthetase (NARS) |
| TCD1_F10 | H. sapiens coatomer protein complex, subunit alpha |
| | (COPA) mRNA |
| TCD1_G10 | H. sapiens enterocyte differentiation associated factor |
| | EDAF-1 mRNA |
| TCD1_A11 | H. sapiens ATP synthase, subunit b-like (ATP-BL) |
| TCD1_D11 | H. sapiens butyrophilin, subfamily 3, member A3 |
| | (BTN3A3) mRNA |
| TCD1_H11 | H. sapiens T cell receptor alpha delta locus |
| TCD1_H7 | H. sapiens ribosomal protein L31, exons |

TABLE 3
T CELL NON-HODGKIN'S LYMPHOMA SUBTRACTED
PCR ™ LIBRARY - NONSPECIFIC TISSUE DRIVER

| Clone No. | Comments |
|---|---|
| TCD2_D7 | Previously Unknown |
| TCD2_E7 | Previously Unknown |
| TCD2_H8 | Previously Unknown |
| TCD2_E5 | Previously Unknown |
| TCD2_B11 | Previously Unknown |
| TCD2_D1 | Previously Unknown |
| TCD2_B3 | Previously Unknown |
| TCD2_D3 | Previously Unknown |
| TCD2_D4 | Previously Unknown |
| TCD2_C5 | Previously Unknown |
| TCD2_G5 | Previously Unknown |
| TCD2_H5 | Previously Unknown |
| TCD2_A6 | Previously Unknown |
| TCD2_G6 | Previously Unknown |
| TCD2_B7 | Previously Unknown |
| TCD2_F8 | Previously Unknown |
| TCD2_G8 | Previously Unknown |
| TCD2_E9 | Previously Unknown |
| TCD2_D10 | Previously Unknown |
| TCD2_H10 | Previously Unknown |
| TCD2_D2 | H. sapiens mRNA for KIAA0855 protein |
| TCD2_D9 | H. sapiens mRNA for KIAA0855 protein |
| TCD2_H1 | H. sapiens mRNA for KIAA0810 protein |
| TCD2_A2 | Human DNA sequence from clone bG279B7 on chr. |
| | 1q25.1-31.1 |
| TCD2_B2 | H. sapiens mRNA for KIAA1049 protein |
| TCD2_H3 | H. sapiens mRNA for KIAA0955 protein |
| TCD2_A4 | H. sapiens chr. 17, clone hRPC.1171_I_10 |
| TCD2_B4 | H. sapiens mRNA for KIAA1068 protein |
| TCD2_B6 | H. sapiens chr. 4 clone B266E3 map 4q25 |
| TCD2_E8 | H. sapiens chr. 11 from 11p15.5 region |
| TCD2_F9 | H. sapiens mRNA for KIAA0926 protein |
| TCD2_E10 | Human DNA seq from clone 328E19 on chr. 1q12-21.2 |
| TCD2_D11 | H. sapiens clone DJ0876A24 |
| TCD2_E1 | Human mRNA for T cell receptor alpha chain (TCR-alpha) |
| TCD2_G3 | Human T-cell receptor active alpha-chain mRNA |
| TCD2_F7 | H. sapiens mRNA for T-cell antigen receptor alpha-chain |
| TCD2_A8 | H. sapiens mRNA for T-cell antigen receptor alpha-chain |
| TCD2_F10 | Human T-cell receptor rearranged alpha-chain V-region |
| TCD2_G10 | Human T-cell receptor active alpha-chain mRNA |
| TCD2_C11 | Human mRNA for T-cell receptor alpha chain |
| TCD2_E11 | Human mRNA for T-cell receptor alpha chain (TCR-alpha) |
| TCD2_G1 | Human T-cell receptor beta |
| TCD2_F4 | Human T-cell receptor beta |
| TCD2_B8 | H. sapiens (clone HVB 15) germline T-cell receptor beta |
| | chain variable seq. |
| TCD2_F3 | H. sapiens interleukin 16 |
| TCD2_C9 | H. sapiens interleukin 16 |
| TCD2_A11 | H. sapiens small inducible cytokine subfamily A (Cys-Cys), |
| | member 21 (SCYA21) |
| TCD2_E12 | H. sapiens small inducible cytokine subfamily A (Cys-Cys), |
| | member 21 (SCYA21) |
| TCD2_E4 | Human mRNA for CD8 beta-chain glycoprotein beta chain |
| TCD2_C8 | Human mRNA for CD8 T lymphocyte surface glycoprotein |
| | beta chain |
| TCD2_F1 | H. sapiens T cell receptor alpha delta locus |
| TCD2_C2 | H. sapiens WD repeat domain 1 (WDR1) mRNA |
| TCD2_E2 | H. sapiens gene for TMEM1 and PWP2 |
| TCD2_F2 | H. sapiens chemokine receptor-4 (CXCR4) mRNA |
| TCD2_G2 | H. sapiens glycogenin-2 like mRNA sequence |
| TCD2_H2 | H. sapiens core-binding factor, runt domain, alpha subunit |
| | 3 (CBFA3) mRNA |
| TCD2_C4 | H. sapiens EWS gene, intron 8 |
| TCD2_G4 | Human GT334 protein (GT334) gene mRNA |
| TCD2_H4 | H. sapiens mRNA for squamous cell carcinoma antigen |
| | SART-3 |
| TCD2_A5 | H. sapiens mRNA for leucocyte adhesion receptor, |
| | L-selectin |
| TCD2_D5 | H. sapiens nuclear factor related to kappa B binding |
| | protein (NFRKB) mRNA |
| TCD2_F5 | H. sapiens T-cell receptor alpha delta locus |
| TCD2_E6 | Human DNA for T-cell receptor constant region |
| | alpha-chain exon4 |
| TCD2_F6 | H. sapiens CD48 antigen |
| TCD2_G7 | H. sapiens CXCR4 gene |
| TCD2_A9 | Human APRT gene for adenine phosphoribosyltransferase |
| TCD2_B9 | Human nuclear pore complex-associated protein TPR (tpr) |
| | mRNA |
| TCD2_H9 | H. sapiens mRNA for YSK1 |
| TCD2_B10 | H. sapiens inositol polyphosphate-5-phosphatase, 145 kD |
| TCD2_C10 | H. sapiens FUS/TLS protein gene, altern. spliced products |
| TCD2_F11 | H. sapiens RH gene, promoter region |
| TCD2_G11 | H. sapiens IL2-inducible T-cell kinase (ITK) mRNA |
| TCD2_H11 | H. sapiens transcription factor 7 (T-cell specific, |
| | HMG-box) (TCF7) |
| TCD2_A12 | Human O-linked GlcNAc transferase mRNA |
| TCD2_B12 | Human tyrosine kinase TXK (txk) gene |
| TCD2_D12 | Human T-cell antigen receptor gene T3 delta |
| TCD2_G12 | H. sapiens proteasome subunit, alpha type, 3 (PSMA3) |
| | mRNA |
| TCD2_H12 | H. sapiens integrin, alpha L (antigen CD11A (p180), |
| | lymphocyte function-assoc.) |
| TCD2_C12 | H. sapiens ribosomal protein S20 (RPS20) mRNA |
| TCD2_H7 | Unknown (sequence withdrawn by NCBI) |
| TCD2_C3 | Human repeat |

TABLE 4
B CELL NON-HODGKIN'S LYMPHOMA SUBTRACTED
PCR ™ LIBRARY - DRIVER #1

| Clone No. | Comments |
|---|---|
| BCNHL/D1_B11 | Previously Unknown |
| BCNHL/D1_F7 | Previously Unknown |
| BCNHL/D1_H4 | Previously Unknown |
| BCNHL/D1_H10 | Previously Unknown |
| BCNHL/D1_H12 | Previously Unknown |
| BCNHL/D1_A3 | Previously Unknown |
| BCNHL/D1_A9 | Previously Unknown |
| BCNHL/D1_A12 | Previously Unknown |
| BCNHL/D1_B1 | Previously Unknown |
| BCNHL/D1_B5 | Previously Unknown |
| BCNHL/D1_B12 | Previously Unknown |
| BCNHL/D1_C1 | Previously Unknown |
| BCNHL/D1_C7 | Previously Unknown |
| BCNHL/D1_D7 | Previously Unknown |
| BCNHL/D1_D8 | Previously Unknown |
| BCNHL/D1_D11 | Previously Unknown |
| BCNHL/D1_E4 | Previously Unknown |
| BCNHL/D1_E7 | Previously Unknown |
| BCNHL/D1_E11 | Previously Unknown |
| BCNHL/D1_G4 | Previously Unknown |
| BCNHL/D1_G5 | Previously Unknown |
| BCNHL/D1_G8 | Previously Unknown |
| BCNHL/D1_H5 | Previously Unknown |
| BCNHL/D1_A4 | cDNA clone DKFZp564C1563, from fetal brain |
| BCNHL/D1_A6 | cDNA clone DKFZp586E1120, from uterus |
| BCNHL/D1_A8 | cDNA clone KIAA0663, from adult brain |
| BCNHL/D1_B9 | Chromosome 19, cosmid R29882 |
| BCNHL/D1_B10 | cDNA clone KIAA1082, from brain |
| BCNHL/D1_D3 | cDNA clone KIAA0084, from myeloblast cell line |
| | KG-1 |
| BCNHL/D1_D4 | cDNA clone 23851, from infant brain |
| BCNHL/D1_D12 | cDNA clone DKFZp434B103, from adult testis |
| BCNHL/D1_E3 | cDNA clone KIAA0008, from myeloblast cell line |
| | KG-1 |
| BCNHL/D1_E12 | cDNA clone DKFZp586J0917, from uterus |
| BCNHL/D1_F6 | Chromosome 1, clone 97P20, Previously Unknown |
| | CDS |
| BCNHL/D1_G3 | cDNA clone KIAA0981, from adult brain |
| BCNHL/D1_H2 | cDNA clone DKFZp434L1435, from adult testis |
| BCNHL/D1_H6 | cDNA clone DKFZp564B0262, from fetal brain |
| BCNHL/D1_H11 | cDNA clone KIAA0372, from brain |
| BCNHL/D1_A7 | CD20 (B1) B lymphocyte cell surface antigen |
| BCNHL/D1_G6 | CD20 (B1) B lymphocyte cell surface antigen |
| BCNHL/D1_H9 | CD20 (B1) B lymphocyte cell surface antigen |
| BCNHL/D1_D6 | Ig lambda light chain |
| BCNHL/D1_E5 | Ig lambda light chain |
| BCNHL/D1_D1 | Lymphoid-restricted membrane protein (LRMP) |
| BCNHL/D1_G12 | Lymphoid-restricted membrane protein (LRMP) |
| BCNHL/D1_A1 | Nucleoporin |
| BCNHL/D1_A5 | Kinesin-related protein |
| BCNHL/D1_B6 | Methyl-CpG binding protein 1 (MBD4) |
| BCNHL/D1_B7 | Heterogeneous nuclear ribonucleoprotein H1 (H) |
| BCNHL/D1_B8 | Ubiquitin-specific protease homolog (UPH) |
| BCNHL/D1_C2 | GTPase activating protein (GAP), 100% 86/423 bp |
| BCNHL/D1_C3 | TCP1 ring complex, polypeptide 5 (TRIC5), |
| | cytoplasmic chaperonin |
| BCNHL/D1_C5 | Nuclear distribution protein C homolog (NUDC) |
| BCNHL/D1_C6 | BAX (apoptosis regulator) |
| BCNHL/D1_C12 | Centromeric autoantigen (27 kD) (P27) |
| BCNHL/D1_D10 | Ig kappa light chain |
| BCNHL/D1_F1 | Serine/Threonine-protein kinase PRP4 homolog |
| BCNHL/D1_F4 | Myocyte-specific enhancer factor 2 (XMEF2) |
| BCNHL/D1_F9 | mRNA for 130 kD protein (p130), Rb family member |
| BCNHL/D1_F10 | CD53 cell surface glycoprotein |
| BCNHL/D1_F11 | Synovial sarcoma, translocated to X chromosome |
| | (SYT..SSXT) |
| BCNHL/D1_F12 | Cyclin B |
| BCNHL/D1_G7 | Regulator of G protein signaling (RGS13) |
| BCNHL/D1_G9 | DEAD/H box polypeptide 16 (DDX16), mRNA |
| | helicase |
| BCNHL/D1_G10 | Pre-mRNA splicing factor (PRP16), a putative helicase |
| BCNHL/D1_G11 | hn ribonucleoprotein D-like gene (JKTBP1/2) |
| BCNHL/D1_H1 | SH2 containing inositol-5-phosphatase (SHIP) |
| BCNHL/D1_H3 | Dystrophin-related protein, utrophin (UTRN) |
| BCNHL/D1_H7 | Inter-alpha-trypsin inhibitor H4 (ITIH4) |
| BCNHL/D1_H8 | Ig heavy chain |

TABLE 5
B CELL NON-HODGKIN'S LYMPHOMA SUBTRACTED
PCR ™ LIBRARY—DRIVER #2

| Clone No. | Comments |
|---|---|
| BCNHL/D2_A4 | Previously Unknown |
| BCNHL/D2_C12 | Previously Unknown |
| BCNHL/D2_D11 | Previously Unknown |
| BCNHL/D2_E6 | Previously Unknown |
| BCNHL/D2_E9 | Previously Unknown |
| BCNHL/D2_E12 | Previously Unknown |
| BCNHL/D2_F4 | Previously Unknown |
| BCNHL/D2_G11 | Previously Unknown |
| BCNHL/D2_H4 | Previously Unknown |
| BCNHL/D2_H11 | Previously Unknown |
| BCNHL/D2_A2 | Previously Unknown |
| BCNHL/D2_A7 | Previously Unknown |
| BCNHL/D2_B2 | Previously Unknown |
| BCNHL/D2_C5 | Previously Unknown |
| BCNHL/D2_C6 | Previously Unknown |
| BCNHL/D2_C11 | Previously Unknown |
| BCNHL/D2_D1 | Previously Unknown |
| BCNHL/D2_D3 | Previously Unknown |
| BCNHL/D2_D12 | Previously Unknown |
| BCNHL/D2_E4 | Previously Unknown |
| BCNHL/D2_E11 | Previously Unknown |
| BCNHL/D2_F3 | Previously Unknown |
| BCNHL/D2_F5 | Previously Unknown |
| BCNHL/D2_F10 | Previously Unknown |
| BCNHL/D2_G7 | Previously Unknown |
| BCNHL/D2_H12 | Previously Unknown |
| BCNHL/D2_B8 | cDNA clone DKFZp586E0518 from uterus |
| | (telomerase, hTLP2) |
| BCNHL/D2_C8 | cDNA clone DKFZp586E0518 from uterus |
| | (telomerase, hTLP2) |
| BCNHL/D2_A5 | cDNA clone KIAA0101 from myeloblast cell line |
| | KG-1 |
| BCNHL/D2_B6 | Chromosome 22 (also chromosome 21 and 4) |
| BCNHL/D2_C2 | cDNA clone DKFZp566L034, from fetal kidney |
| BCNHL/D2_C3 | Chromosome 16, clone RPCI-11 |
| BCNHL/D2_C10 | cDNA clone KJAA0121 from myeloblast cell line |
| | KG-1 |
| BCNHL/D2_F11 | cDNA clone KIAA0185 (KG-1); apoptosis-linked |
| | gene 4 (Alg-4) |
| BCNHL/D2_G8 | cDNA clone DKFZp434C171, from adult testis |
| BCNHL/D2_G9 | cDNA clone KIAA0209, from myeloblast cell line |
| | KG-1 |
| BCNHL/D2_H8 | cDNA clone KIAA0855, from adult brain |
| BCNHL/D2_H10 | Chromosome 19, cosmid R28051 |
| BCNHL/D2_B1 | Ig lambda light chain |
| BCNHL/D2_C1 | Ig lambda light chain |
| BCNHL/D2_C4 | Ig lambda light chain |
| BCNHL/D2_D8 | Ig lambda light chain |
| BCNHL/D2_E7 | Ig lambda light chain |
| BCNHL/D2_E8 | Ig lambda light chain |
| BCNHL/D2_F8 | Ig lambda light chain |
| BCNHL/D2_G4 | Ig lambda light chain |
| BCNHL/D2_H3 | Ig lambda light chain |
| BCNHL/D2_A8 | Ig kappa light chain (82% identity) |
| BCNHL/D2_H7 | Ig kappa light chain |
| BCNHL/D2_A10 | CD20 (B1) B lymphocyte cell-surface antigen |
| BCNHL/D2_E5 | CD20 (B1) B lymphocyte cell-surface antigen |
| BCNHL/D2_A6 | CD37 antigen (CD37) |
| BCNHL/D2_A12 | 5′-end (221/408) is 100% part of histone deacetylase |
| | (HD1) CDS |
| BCNHL/D2_B5 | p56Ick (Ick), protein tyrosine kinase (membrane) |
| BCNHL/D2_B7 | Lymphoid-restricted membrane protein |
| BCNHL/D2_B9 | Interferon consensus sequence binding protein 1 |
| | (ICSBP1) |
| BCNHL/D2_C7 | Dp-1 transcription factor (TFDP1) |
| BCNHL/D2_D10 | Transcription termination factor, RNA polymerase II |
| | (TTF2) |
| BCNHL/D2_E2 | BCL2-related protein A1 (BCL2A1) |
| BCNHL/D2_E10 | RNA helicase p68 (HUMP68) |
| BCNHL/D2_F7 | Phosphate carrier, mitochondrial (PHC), nt#1-138; |
| | SWAP-70 (Ig switching), nt#135-311 |
| BCNHL/D2_F9 | TNF-induced protein (GG2-1); dendritic cell |
| | differentiation factor |
| BCNHL/D2_G3 | Hepatocyte nuclear factor-3/forke head homolog |
| | 11B (HFH-11B) |
| BCNHL/D2_G5 | MHC class II HLA-DQA1 |
| BCNHL/D2_G6 | 90 kD heat shock protein |
| BCNHL/D2_G12 | 5′-end (120/347) is 100% part of Gamma 2-adaptin |
| | (G2AD) CDS |
| BCNHL/H5_H5 | Ras homolog gene family, member H (ARHH) |

TABLE 6
HODGKIN'S LYMPHOMA SUBTRACTED PCR ™ LIBRARY

| Clone No. | Comments |
|---|---|
| HLS_E3 | Previously Unknown |
| HLS_C4 | Previously Unknown |
| HLS_G8 | Previously Unknown |
| HLS_D11 | Previously Unknown |
| HLS_C1 | Previously Unknown |
| HLS_E1 | Previously Unknown |
| HLS_B2 | Previously Unknown |
| HLS_A3 | Previously Unknown |
| HLS_G3 | Previously Unknown |
| HLS_H4 | Previously Unknown |
| HLS_H5 | Previously Unknown |
| HLS_D6 | Previously Unknown |
| HLS_H7 | Previously Unknown |
| HLS_B8 | Previously Unknown |
| HLS_C8 | Previously Unknown |
| HLS_D8 | Previously Unknown |
| HLS_F9 | Previously Unknown |
| HLS_F11 | Previously Unknown |
| HLS_E5 | Previously Unknown |
| HLS_B7 | Previously Unknown |
| HLS_H9 | Previously Unknown |
| HLS_H10 | Previously Unknown |
| HLS_H1 | Human mRNA for KIAA0143 gene |
| HLS_E2 | H. sapiens DNA seq from PAC 163M9 on chr 1p35.1-p36.21. |
| HLS_H3 | Human DNA seq ft clone CTA-407F11 on chr. 22q12 |
| HLS_G5 | Human HMG-17 gene for non-histone chr. protein HMG-17 |
| HLS_B6 | Human Chr. 11q12.2 PAC clone pDJ606g6 |
| HLS_H6 | H. sapiens mRNA; cDNA DKFZp564A132 |
| HLS_D7 | Human DNA sequence from clone RP1-506 on chr 22q12 |
| HLS_E7 | H. sapiens chr. 17, clone hRPC.1028_K_7 |
| HLS_F8 | H. sapiens 12p13.3-2.7-4.6 BAC RP11-372B4 |
| HLS_H8 | Human Chr. 16 BAC clone CIT987SK-A-355G7 |
| HLS_A9 | H. sapiens PAC clone DJ0320J15 from Xq23 |
| HLS_B9 | Human interferon-inducible mRNA (cDNA 6-26) |
| HLS_C12 | Human DNA seq ft clone RP1-90L6 on chr. 22q11.21-11.23 |
| HLS_D12 | Human Chr. 16 BAC clone CIT987SK-A-735G6 |
| HLS_E12 | H. sapiens hypothetical protein SBBI42 mRNA |
| HLS_F12 | H. sapiens DNA sequence from PAC 747L4 on chr. 1 q23-24 |
| HLS_G12 | H. sapiens mRNA; cDNA DKFZp586H0519 |
| HLS_H12 | H. sapiens clone 25114 mRNA sequence |
| HLS_G1 | H. sapiens mRNA for KIAA0776 protein |
| HLS_A7 | H. sapiens mRNA for KIAA0776 protein |
| HLS_A1 | H. sapiens protective protein for beta-galactosidase |
| HLS_B1 | Human proliferating cell nuclear antigen (PCNA) gene |
| HLS_A2 | Human mRNA for myoblast cell surface antigen 24.1D5 |
| HLS_F2 | Human mRNA for interferon regulatory factor-2 (IRF-2) |
| HLS_C3 | H. sapiens ADP/ATP carrier protein (ANT-2) gene |
| HLS_F3 | Human GDP-dissociation inhibitor protein (Ly-GDI) mRNA |
| HLS_A4 | H. sapiens microfibrillar-associated protein 1 (MFAP1) |
| | mRNA |
| HLS_B4 | H. sapiens caspase 3, apoptosis-related cysteine protease |
| | (CASP3) |
| HLS_D4 | Human thymosin beta-4 mRNA, complete cds |
| HLS_E4 | Human lymphocyte specific INF regul. factor/INF reg. |
| | factor 4 (LSIRF/IRF4) |
| HLS_F4 | H. sapiens integrin, beta 1 (fibronectin receptor, antigen |
| | CD29) (ITGB1) |
| HLS_G4 | H. sapiens proteasome (prosome, macropain) subunit, alpha |
| | type, 3 (PSMA3) |
| HLS_A5 | H. sapiens mRNA for Prer protein |
| HLS_B5 | H. sapiens purinergic receptor P2X, ligand-gated ion |
| | channel, 5 (P2RX5) |
| HLS_D5 | H. sapiens IRLB gene (3′-region) |
| HLS_A6 | H. sapiens initiation factor 4B cDNA |
| HLS_C6 | Human poly(A)-binding protein (PABP) gene, exon 15 |
| HLS_G6 | Rat proto-oncogene (Ets-1) mRNA, complete cds |
| HLS_G7 | Human 78 kdalton glucose-regulated protein (GRP78) gene |
| HLS_A8 | Human t-complex polypeptide 1 gene |
| HLS_E8 | Human TRAF-interacting protein 1-TRAF mRNA |
| HLS_C9 | H. sapiens collagen, type III, alpha 1 (Ehlers-Danlos |
| | syndrome type IV) |
| HLS_D9 | H. sapiens E46 protein mRNA, complete cds |
| HLS_E9 | H. sapiens chromodomain helicase DNA binding protein 4 |
| | (CHD4) |
| HLS_G9 | H. sapiens DNA for monoamine oxidase type A (14) (partial) |
| HLS_A10 | H. sapiens ATP binding protein assoc. with cell |
| | differentiation (APACD) |
| HLS_D10 | Human non-histone dir. protein HMG-14 gene, complete cds |
| HLS_F10 | Human protein phosphatase-1 gamma 1 mRNA |
| HLS_C11 | Human hnRNP B1 protein mRNA |
| HLS_E11 | H. sapiens epithelial protein lost in neoplasm alpha (EPLIN) |
| HLS_G11 | Human ferritin heavy chain mRNA |
| HLS_H11 | H. sapiens foocen-s mRNA |
| HLS_B12 | Human myocyte-specific enhancer factor 2A (MEF2A) gene |
| HLS_D1 | H. sapiens osf-2 mRNA for osteoblast specific factor 2 |
| | (OSF-2p1) |
| HLS_H2 | H. sapiens osf-2 mRNA for osteoblast specific factor 2 |
| | (OSF-2p1) |
| HLS_D3 | H. sapiens osf-2 mRNA for osteoblast specific factor 2 |
| | (OSF-2p1) |
| HLS_B10 | H. sapiens osf-2 mRNA for osteoblast specific factor 2 |
| | (OSF-2p1) |
| HLS_C10 | H. sapiens osf-2 mRNA for osteoblast specific factor 2 |
| | (OSF-2p1) |
| HLS_G10 | H. sapiens osf-2 mRNA for osteoblast specific factor 2 |
| | (OSF-2p1) |
| HLS_F1 | Hu Ig superfamily cytotoxic T-lymphocyte-assoc. protein |
| | (CTLA-4) gene |
| HLS_C2 | Hu Ig superfamily cytotoxic T-lymphocyte-assoc. protein |
| | (CTLA-4) gene |
| HLS_G2 | H. sapiens beta-2-microglobulin (B2M) mRNA |
| HLS_F6 | H. sapiens beta-2-microglobulin (B2M) mRNA |
| HLS_C5 | Hu common acute lymphoblastic leukemia antigen (CALLA) |
| HLS_C7 | Hu common acute lymphoblastic leukemia antigen (CALLA) |
| HLS_E10 | H. sapiens B-cell-homing chemokine (ligand for Burkitt's |
| | lymp. Receptor-1) (BLC) |
| HLS_A11 | H. sapiens B-cell-homing chemokine (ligand for Burkitt's |
| | lymp. Receptor-1) (BLC) |
| HLS_D2 | H. sapiens genes for ribosomal protein L13a |
| HLS_F5 | H. sapiens ribosomal protein S7 (RPS7) |
| HLS_F7 | H. sapiens ribosomal protein S17 (RPS17) mRNA |
| HLS_A12 | H. sapiens ribosomal protein S17 (RPS17) mRNA |

### Example 2

### Analysis of Subtracted cDNA Sequences by Microarray Analysis

Subtracted cDNA sequences were analyzed by microarray analysis to evaluate their expression in hematological malignancies and normal tissues. Using this approach, cDNA sequences were PCR amplified and their mRNA expression profiles in hematological malignancies and normal tissues are examined using cDNA microarray technology essentially as described (Shena et al., 1995).

In brief, the clones identified from the subtracted cDNA libraries analyses were immobilized and arrayed onto glass slides as multiple replicas on microarray slides and the slides were hybridized with two different sets of probes. , with each location corresponding to a unique cDNA clone (as many as 5500 clones can be arrayed on a single slide, or chip). Each chip is hybridized with a pair of cDNA probes that are fluorescence-labeled with Cy3 and Cy5, respectively. The set of probes derived from the hematological malignancies was labeled with cy3 while the other set of probes derived from a pool of normal tissues was labeled with cy5. Typically, 1 μg of polyA⁺ RNA was used to generate each cDNA probe. After hybridization, the chips were scanned and the fluorescence intensity recorded for both Cy3 and Cy5 channels. The difference in intensities (i.e., cy3/cy5 ratios) following hybridization with both probe sets provided the information on the relative expression level of each cDNA sequences immobilized on the slide in tumor versus normal tissues. There are multiple built-in quality control steps. First, the probe quality is monitored using a panel of ubiquitously expressed genes. Secondly, the control plate also can include yeast DNA fragments of which complementary RNA may be spiked into the probe synthesis for measuring the quality of the probe and the sensitivity of the analysis. This methodology provides a sensitivity of 1 in 100,000 copies of mRNA, and the reproducibility of the technology may be ensured by including duplicated control cDNA elements at different locations.

Analysis of hematological malignancy subtracted clones by microarray analyses on a variety of microarray chips identified the sequences set forth in SEQ ID NO:1 through SEQ ID NO:668 as being at least two-fold overexpressed in hematological malignancies versus normal tissues.

### Example 3

### Polynucleotide and Polypeptide Compositions: Brief Description of the cDNA Clones and Open Reading Frames Identified by Subtractive Hybridization and Microarray Analysis

Table 7 lists the sequences of the polynucleotides obtained during the analyses of the present invention. Shown are the 669 polynucleotide sequences, along with their clone name identifiers, as well as the serial number and filing date of the priority provisional patent application in which the clone was first identified.

TABLE 7
POLYNUCLEOTIDE SEQUENCES OF THE PRESENT INVENTION

| | | Priority | |
|---|---|---|---|
| | | Application | |
| SEQ ID NO: | Clone Identifier | Number | Filing Date |
| SEQ ID NO:1 | ′41567.1_gaiger.ABI′ | 60/190,479 | Mar. 17, 2000 |
| SEQ ID NO:2 | ′41557.1_gaiger.ABI′ | 60/190,479 | Mar. 17, 2000 |
| SEQ ID NO:3 | ′41577.1_gaiger.ABI′ | 60/190,479 | Mar. 17, 2000 |
| SEQ ID NO:4 | ′41571.1_gaiger.ABI′ | 60/190,479 | Mar. 17, 2000 |
| SEQ ID NO:5 | ′41594.1_gaiger.ABI′ | 60/190,479 | Mar. 17, 2000 |
| SEQ ID NO:6 | ′41605.1_gaiger.ABI′ | 60/190,479 | Mar. 17, 2000 |
| SEQ ID NO:7 | ′41627.1_gaiger.ABI′ | 60/190,479 | Mar. 17, 2000 |
| SEQ ID NO:8 | ′41620.1_gaiger.ABI′ | 60/190,479 | Mar. 17, 2000 |
| SEQ ID NO:9 | ′41628.1_gaiger.ABI′ | 60/190,479 | Mar. 17, 2000 |
| SEQ ID NO:10 | ′41635.1_gaiger.ABI′ | 60/190,479 | Mar. 17, 2000 |
| SEQ ID NO:11 | ′41649.1_gaiger.ABI′ | 60/190,479 | Mar. 17, 2000 |
| SEQ ID NO:12 | ′41648.1_gaiger.ABI′ | 60/190,479 | Mar. 17, 2000 |
| SEQ ID NO:13 | ′41653.1_gaiger.ABI′ | 60/190,479 | Mar. 17, 2000 |
| SEQ ID NO:14 | ′41664.1_gaiger.ABI′ | 60/190,479 | Mar. 17, 2000 |
| SEQ ID NO:15 | ′41667.1_gaiger.ABI′ | 60/190,479 | Mar. 17, 2000 |
| SEQ ID NO:16 | ′41687.1_gaiger.ABI′ | 60/190,479 | Mar. 17, 2000 |
| SEQ ID NO:17 | ′41708.1_gaiger.ABI′ | 60/190,479 | Mar. 17, 2000 |
| SEQ ID NO:18 | ′41721.1_gaiger.ABI′ | 60/190,479 | Mar. 17, 2000 |
| SEQ ID NO:19 | ′41746.1_gaiger.ABI′ | 60/190,479 | Mar. 17, 2000 |
| SEQ ID NO:20 | ′41751.1_gaiger.ABI′ | 60/190,479 | Mar. 17, 2000 |
| SEQ ID NO:21 | ′41762.1_gaiger.ABI′ | 60/190,479 | Mar. 17, 2000 |
| SEQ ID NO:22 | ′41764.1_gaiger.ABI′ | 60/190,479 | Mar. 17, 2000 |
| SEQ ID NO:23 | ′41793.1_gaiger.ABI′ | 60/190,479 | Mar. 17, 2000 |
| SEQ ID NO:24 | ′41794.1_gaiger.ABI′ | 60/190,479 | Mar. 17, 2000 |
| SEQ ID NO:25 | ′41807.1_gaiger.ABI′ | 60/190,479 | Mar. 17, 2000 |
| SEQ ID NO:26 | ′41802.1_gaiger.ABI′ | 60/190,479 | Mar. 17, 2000 |
| SEQ ID NO:27 | ′41804.1_gaiger.ABI′ | 60/190,479 | Mar. 17, 2000 |
| SEQ ID NO:28 | ′41810.1_gaiger.ABI′ | 60/190,479 | Mar. 17, 2000 |
| SEQ ID NO:29 | ′41847.1_gaiger.ABI′ | 60/190,479 | Mar. 17, 2000 |
| SEQ ID NO:30 | ′41865.1_gaiger.ABI′ | 60/190,479 | Mar. 17, 2000 |
| SEQ ID NO:31 | ′41859.1_gaiger.ABI′ | 60/190,479 | Mar. 17, 2000 |
| SEQ ID NO:32 | ′41878.1_gaiger.ABI′ | 60/190,479 | Mar. 17, 2000 |
| SEQ ID NO:33 | ′41869.1_gaiger.ABI′ | 60/190,479 | Mar. 17, 2000 |
| SEQ ID NO:34 | ′41888.1_gaiger.ABI′ | 60/190,479 | Mar. 17, 2000 |
| SEQ ID NO:35 | ′41907.1_gaiger.ABI′ | 60/190,479 | Mar. 17, 2000 |
| SEQ ID NO:36 | ′41908.1_gaiger.ABI′ | 60/190,479 | Mar. 17, 2000 |
| SEQ ID NO:37 | ′41912.1_gaiger.ABI′ | 60/190,479 | Mar. 17, 2000 |
| SEQ ID NO:38 | ′41916.1_gaiger.ABI′ | 60/190,479 | Mar. 17, 2000 |
| SEQ ID NO:39 | ′41925.1_gaiger.ABI′ | 60/190,479 | Mar. 17, 2000 |
| SEQ ID NO:40 | ′41929.1_gaiger.ABI′ | 60/190,479 | Mar. 17, 2000 |
| SEQ ID NO:41 | ′41930.1_gaiger.ABI′ | 60/190,479 | Mar. 17, 2000 |
| SEQ ID NO:42 | ′41933.1_gaiger.ABI′ | 60/190,479 | Mar. 17, 2000 |
| SEQ ID NO:43 | ′41944.1_gaiger.ABI′ | 60/190,479 | Mar. 17, 2000 |
| SEQ ID NO:44 | ′41986.1_gaiger.ABI′ | 60/190,479 | Mar. 17, 2000 |
| SEQ ID NO:45 | ′42017.1_gaiger.ABI′ | 60/190,479 | Mar. 17, 2000 |
| SEQ ID NO:46 | ′42033.1_gaiger.ABI′ | 60/190,479 | Mar. 17, 2000 |
| SEQ ID NO:47 | ′42040.1_gaiger.ABI′ | 60/190,479 | Mar. 17, 2000 |
| SEQ ID NO:48 | ′42041.1_gaiger.ABI′ | 60/190,479 | Mar. 17, 2000 |
| SEQ ID NO:49 | ′42053.1_gaiger.ABI′ | 60/190,479 | Mar. 17, 2000 |
| SEQ ID NO:50 | ′42101.1_gaiger.ABI′ | 60/190,479 | Mar. 17, 2000 |
| SEQ ID NO:51 | ′42131.1_gaiger.ABI′ | 60/190,479 | Mar. 17, 2000 |
| SEQ ID NO:52 | R0232:A08 | 60/206,201 | May 22, 2000 |
| SEQ ID NO:53 | R0232:C10 | 60/206,201 | May 22, 2000 |
| SEQ ID NO:54 | R0232:H11 | 60/206,201 | May 22, 2000 |
| SEQ ID NO:55 | R0232:H03 | 60/206,201 | May 22, 2000 |
| SEQ ID NO:56 | R0233:A12 | 60/206,201 | May 22, 2000 |
| SEQ ID NO:57 | R0233:A06 | 60/206,201 | May 22, 2000 |
| SEQ ID NO:58 | R0233:A08 | 60/206,201 | May 22, 2000 |
| SEQ ID NO:59 | R0233:B10 | 60/206,201 | May 22, 2000 |
| SEQ ID NO:60 | R0233:B04 | 60/206,201 | May 22, 2000 |
| SEQ ID NO:61 | R0233:C04 | 60/206,201 | May 22, 2000 |
| SEQ ID NO:62 | R0233:D01 | 60/206,201 | May 22, 2000 |
| SEQ ID NO:63 | R0233:D02 | 60/206,201 | May 22, 2000 |
| SEQ ID NO:64 | R0233:F10 | 60/206,201 | May 22, 2000 |
| SEQ ID NO:65 | R0233:F05 | 60/206,201 | May 22, 2000 |
| SEQ ID NO:66 | R0233:F07 | 60/206,201 | May 22, 2000 |
| SEQ ID NO:67 | ′42324.1_gaiger.ABI′ | 60/200,779 | May 22, 2000 |
| SEQ ID NO:68 | ′42349.1_gaiger.ABI′ | 60/200,779 | May 22, 2000 |
| SEQ ID NO:69 | ′42379.1_gaiger.ABI′ | 60/200,779 | May 22, 2000 |
| SEQ ID NO:70 | ′42394.1_gaiger.ABI′ | 60/200,779 | May 22, 2000 |
| SEQ ID NO:71 | ′42387.1_gaiger.ABI′ | 60/200,779 | May 22, 2000 |
| SEQ ID NO:72 | ′42396.1_gaiger.ABI′ | 60/200,779 | May 22, 2000 |
| SEQ ID NO:73 | ′42424.1_gaiger.ABI′ | 60/200,779 | May 22, 2000 |
| SEQ ID NO:74 | ′42438.1_gaiger.ABI′ | 60/200,779 | May 22, 2000 |
| SEQ ID NO:75 | ′42447.1_gaiger.ABI′ | 60/200,779 | May 22, 2000 |
| SEQ ID NO:76 | ′42524.1;gaiger.ABI′ | 60/200,779 | May 22, 2000 |
| SEQ ID NO:77 | ′42555.1;gaiger.ABI′ | 60/200,779 | May 22, 2000 |
| SEQ ID NO:78 | ′42560.1;gaiger.ABI′ | 60/200,779 | May 22, 2000 |
| SEQ ID NO:79 | ′42594.1_gaiger.ABI′ | 60/200,779 | May 22, 2000 |
| SEQ ID NO:80 | ′42595.1_gaiger.ABI′ | 60/200,779 | May 22, 2000 |
| SEQ ID NO:81 | ′42602.1_gaiger.ABI′ | 60/200,779 | May 22, 2000 |
| SEQ ID NO:82 | ′42665.1_gaiger.ABI′ | 60/200,779 | May 22, 2000 |
| SEQ ID NO:83 | ′42703.1_gaiger.ABI′ | 60/200,779 | May 22, 2000 |
| SEQ ID NO:84 | ′42709.1_gaiger.ABI′ | 60/200,779 | May 22, 2000 |
| SEQ ID NO:85 | ′42756.1_gaiger.ABI′ | 60/200,779 | May 22, 2000 |
| SEQ ID NO:86 | ′42802.1_gaiger.ABI′ | 60/200,779 | May 22, 2000 |
| SEQ ID NO:87 | R0234:A06 | 60/206,201 | May 22, 2000 |
| SEQ ID NO:88 | R0234:A07 | 60/206,201 | May 22, 2000 |
| SEQ ID NO:89 | R0234:B03 | 60/206,201 | May 22, 2000 |
| SEQ ID NO:90 | R0234:B06 | 60/206,201 | May 22, 2000 |
| SEQ ID NO:91 | R0234:B09 | 60/206,201 | May 22, 2000 |
| SEQ ID NO:92 | R0234:C02 | 60/206,201 | May 22, 2000 |
| SEQ ID NO:93 | R0234:C06 | 60/206,201 | May 22, 2000 |
| SEQ ID NO:94 | R0234:D06 | 60/206,201 | May 22, 2000 |
| SEQ ID NO:95 | R0234:D08 | 60/206,201 | May 22, 2000 |
| SEQ ID NO:96 | R0234:E01 | 60/206,201 | May 22, 2000 |
| SEQ ID NO:97 | R0234:E12 | 60/206,201 | May 22, 2000 |
| SEQ ID NO:98 | R0234:E02 | 60/206,201 | May 22, 2000 |
| SEQ ID NO:99 | R0234:E04 | 60/206,201 | May 22, 2000 |
| SEQ ID NO:100 | R0234:E05 | 60/206,201 | May 22, 2000 |
| SEQ ID NO:101 | R0234:F01 | 60/206,201 | May 22, 2000 |
| SEQ ID NO:102 | R0234:F02 | 60/206,201 | May 22, 2000 |
| SEQ ID NO:103 | R0234:F04 | 60/206,201 | May 22, 2000 |
| SEQ ID NO:104 | R0234:G01 | 60/206,201 | May 22, 2000 |
| SEQ ID NO:105 | R0234:G11 | 60/206,201 | May 22, 2000 |
| SEQ ID NO:106 | R0234:G12 | 60/206,201 | May 22, 2000 |
| SEQ ID NO:107 | R0234:G02 | 60/206,201 | May 22, 2000 |
| SEQ ID NO:108 | R0234:G03 | 60/206,201 | May 22, 2000 |
| SEQ ID NO:109 | R0234:G04 | 60/206,201 | May 22, 2000 |
| SEQ ID NO:110 | R0234:G09 | 60/206,201 | May 22, 2000 |
| SEQ ID NO:111 | R0234:H01 | 60/206,201 | May 22, 2000 |
| SEQ ID NO:112 | R0234:H06 | 60/206,201 | May 22, 2000 |
| SEQ ID NO:113 | R0235:A11 | 60/206,201 | May 22, 2000 |
| SEQ ID NO:114 | R0235:A07 | 60/206,201 | May 22, 2000 |
| SEQ ID NO:115 | R0235:B01 | 60/206,201 | May 22, 2000 |
| SEQ ID NO:116 | R0235:B11 | 60/206,201 | May 22, 2000 |
| SEQ ID NO:117 | R0235:B04 | 60/206,201 | May 22, 2000 |
| SEQ ID NO:118 | R0235:B05 | 60/206,201 | May 22, 2000 |
| SEQ ID NO:119 | R0235:B07 | 60/206,201 | May 22, 2000 |
| SEQ ID NO:120 | R0235:B09 | 60/206,201 | May 22, 2000 |
| SEQ ID NO:121 | R0235:C07 | 60/206,201 | May 22, 2000 |
| SEQ ID NO:122 | R0235:C09 | 60/206,201 | May 22, 2000 |
| SEQ ID NO:123 | R0235:D11 | 60/206,201 | May 22, 2000 |
| SEQ ID NO:124 | R0235:E10 | 60/206,201 | May 22, 2000 |
| SEQ ID NO:125 | R0235:E12 | 60/206,201 | May 22, 2000 |
| SEQ ID NO:126 | R0235:E02 | 60/206,201 | May 22, 2000 |
| SEQ ID NO:127 | R0235:F01 | 60/206,201 | May 22, 2000 |
| SEQ ID NO:128 | R0235:F02 | 60/206,201 | May 22, 2000 |
| SEQ ID NO:129 | R0235:F06 | 60/206,201 | May 22, 2000 |
| SEQ ID NO:130 | R0235:F07 | 60/206,201 | May 22, 2000 |
| SEQ ID NO:131 | R0235:F09 | 60/206,201 | May 22, 2000 |
| SEQ ID NO:132 | R0235:G07 | 60/206,201 | May 22, 2000 |
| SEQ ID NO:133 | R0235:H06 | 60/206,201 | May 22, 2000 |
| SEQ ID NO:134 | R0235:H08 | 60/206,201 | May 22, 2000 |
| SEQ ID NO:135 | R0236:A06 | 60/206,201 | May 22, 2000 |
| SEQ ID NO:136 | R0236:A09 | 60/206,201 | May 22, 2000 |
| SEQ ID NO:137 | R0236:B06 | 60/206,201 | May 22, 2000 |
| SEQ ID NO:138 | R0236:C01 | 60/206,201 | May 22, 2000 |
| SEQ ID NO:139 | R0236:E05 | 60/206,201 | May 22, 2000 |
| SEQ ID NO:140 | R0236:F12 | 60/206,201 | May 22, 2000 |
| SEQ ID NO:141 | R0236:F05 | 60/206,201 | May 22, 2000 |
| SEQ ID NO:142 | R0236:F06 | 60/206,201 | May 22, 2000 |
| SEQ ID NO:143 | R0236:G08 | 60/206,201 | May 22, 2000 |
| SEQ ID NO:144 | R0249:A11 | 60/222,903 | Aug. 03, 2000 |
| SEQ ID NO:145 | R0249:B02 | 60/222,903 | Aug. 03, 2000 |
| SEQ ID NO:146 | R0249:B04 | 60/222,903 | Aug. 03, 2000 |
| SEQ ID NO:147 | R0249:B06 | 60/222,903 | Aug. 03, 2000 |
| SEQ ID NO:148 | R0249:D11 | 60/222,903 | Aug. 03, 2000 |
| SEQ ID NO:149 | R0249:E11 | 60/222,903 | Aug. 03, 2000 |
| SEQ ID NO:150 | R0249:E06 | 60/222,903 | Aug. 03, 2000 |
| SEQ ID NO:151 | R0249:H09 | 60/222,903 | Aug. 03, 2000 |
| SEQ ID NO:152 | R0250:C09 | 60/222,903 | Aug. 03, 2000 |
| SEQ ID NO:153 | R0250:D10 | 60/222,903 | Aug. 03, 2000 |
| SEQ ID NO:154 | R0250:D03 | 60/222,903 | Aug. 03, 2000 |
| SEQ ID NO:155 | R0250:E09 | 60/222,903 | Aug. 03, 2000 |
| SEQ ID NO:156 | R0250:F09 | 60/222,903 | Aug. 03, 2000 |
| SEQ ID NO:157 | R0250:G01 | 60/222,903 | Aug. 03, 2000 |
| SEQ ID NO:158 | R0251:A12 | 60/222,903 | Aug. 03, 2000 |
| SEQ ID NO:159 | R0251:A05 | 60/222,903 | Aug. 03, 2000 |
| SEQ ID NO:160 | R0251:B09 | 60/222,903 | Aug. 03, 2000 |
| SEQ ID NO:161 | R0251:D01 | 60/222,903 | Aug. 03, 2000 |
| SEQ ID NO:162 | R0251:E03 | 60/222,903 | Aug. 03, 2000 |
| SEQ ID NO:163 | R0251:E06 | 60/222,903 | Aug. 03, 2000 |
| SEQ ID NO:164 | R0251:F12 | 60/222,903 | Aug. 03, 2000 |
| SEQ ID NO:165 | R0251:G06 | 60/222,903 | Aug. 03, 2000 |
| SEQ ID NO:166 | R0252:A08 | 60/222,903 | Aug. 03, 2000 |
| SEQ ID NO:167 | R0252:D02 | 60/222,903 | Aug. 03, 2000 |
| SEQ ID NO:168 | R0252:E11 | 60/222,903 | Aug. 03, 2000 |
| SEQ ID NO:169 | R0252:E04 | 60/222,903 | Aug. 03, 2000 |
| SEQ ID NO:170 | R0252:E06 | 60/222,903 | Aug. 03, 2000 |
| SEQ ID NO:171 | R0252:E07 | 60/222,903 | Aug. 03, 2000 |
| SEQ ID NO:172 | R0252:F11 | 60/222,903 | Aug. 03, 2000 |
| SEQ ID NO:173 | R0252:F02 | 60/222,903 | Aug. 03, 2000 |
| SEQ ID NO:174 | R0252:F03 | 60/222,903 | Aug. 03, 2000 |
| SEQ ID NO:175 | R0252:H01 | 60/222,903 | Aug. 03, 2000 |
| SEQ ID NO:176 | R0252:H03 | 60/222,903 | Aug. 03, 2000 |
| SEQ ID NO:177 | R0253:B04 | 60/222,903 | Aug. 03, 2000 |
| SEQ ID NO:178 | R0253:C10 | 60/222,903 | Aug. 03, 2000 |
| SEQ ID NO:179 | R0253:C04 | 60/222,903 | Aug. 03, 2000 |
| SEQ ID NO:180 | R0253:C05 | 60/222,903 | Aug. 03, 2000 |
| SEQ ID NO:181 | R0253:C06 | 60/222,903 | Aug. 03, 2000 |
| SEQ ID NO:182 | R0253:D02 | 60/222,903 | Aug. 03, 2000 |
| SEQ ID NO:183 | R0253:D08 | 60/222,903 | Aug. 03, 2000 |
| SEQ ID NO:184 | R0253:E06 | 60/222,903 | Aug. 03, 2000 |
| SEQ ID NO:185 | R0253:E09 | 60/222,903 | Aug. 03, 2000 |
| SEQ ID NO:186 | R0253:F01 | 60/222,903 | Aug. 03, 2000 |
| SEQ ID NO:187 | R0253:F11 | 60/222,903 | Aug. 03, 2000 |
| SEQ ID NO:188 | R0253:F02 | 60/222,903 | Aug. 03, 2000 |
| SEQ ID NO:189 | R0253:F05 | 60/222,903 | Aug. 03, 2000 |
| SEQ ID NO:190 | R0253:F07 | 60/222,903 | Aug. 03, 2000 |
| SEQ ID NO:191 | R0253:G01 | 60/222,903 | Aug. 03, 2000 |
| SEQ ID NO:192 | R0253:G10 | 60/222,903 | Aug. 03, 2000 |
| SEQ ID NO:193 | R0253:G11 | 60/222,903 | Aug. 03, 2000 |
| SEQ ID NO:194 | R0253:G12 | 60/222,903 | Aug. 03, 2000 |
| SEQ ID NO:195 | R0253:G04 | 60/222,903 | Aug. 03, 2000 |
| SEQ ID NO:196 | R0253:G05 | 60/222,903 | Aug. 03, 2000 |
| SEQ ID NO:197 | R0253:G06 | 60/222,903 | Aug. 03, 2000 |
| SEQ ID NO:198 | R0253:H02 | 60/222,903 | Aug. 03, 2000 |
| SEQ ID NO:199 | R0253:H07 | 60/222,903 | Aug. 03, 2000 |
| SEQ ID NO:200 | R0254:F07 | 60/223,416 | Aug. 04, 2000 |
| SEQ ID NO:201 | R0254:G11 | 60/223,416 | Aug. 04, 2000 |
| SEQ ID NO:202 | R0254:G04 | 60/223,416 | Aug. 04, 2000 |
| SEQ ID NO:203 | R0254:H01 | 60/223,416 | Aug. 04, 2000 |
| SEQ ID NO:204 | R0238:C03 | 60/223,416 | Aug. 04, 2000 |
| SEQ ID NO:205 | R0255:C02 | 60/223,416 | Aug. 04, 2000 |
| SEQ ID NO:206 | R0255:F12 | 60/223,416 | Aug. 04, 2000 |
| SEQ ID NO:207 | R0258:G10 | 60/223,416 | Aug. 04, 2000 |
| SEQ ID NO:208 | R0261:A12 | 60/223,416 | Aug. 04, 2000 |
| SEQ ID NO:209 | R0261:A09 | 60/223,416 | Aug. 04, 2000 |
| SEQ ID NO:210 | R0261:B12 | 60/223,416 | Aug. 04, 2000 |
| SEQ ID NO:211 | R0261:C10 | 60/223,416 | Aug. 04, 2000 |
| SEQ ID NO:212 | R0261:D06 | 60/223,416 | Aug. 04, 2000 |
| SEQ ID NO:213 | R0261:E04 | 60/223,416 | Aug. 04, 2000 |
| SEQ ID NO:214 | R0261:F05 | 60/223,416 | Aug. 04, 2000 |
| SEQ ID NO:215 | R0261:G04 | 60/223,416 | Aug. 04, 2000 |
| SEQ ID NO:216 | R0261:H03 | 60/223,416 | Aug. 04, 2000 |
| SEQ ID NO:217 | R0262:A12 | 60/223,416 | Aug. 04, 2000 |
| SEQ ID NO:218 | R0262:A02 | 60/223,416 | Aug. 04, 2000 |
| SEQ ID NO:219 | R0262:D12 | 60/223,416 | Aug. 04, 2000 |
| SEQ ID NO:220 | R0262:D04 | 60/223,416 | Aug. 04, 2000 |
| SEQ ID NO:221 | R0262:D07 | 60/223,416 | Aug. 04, 2000 |
| SEQ ID NO:222 | R0262:E02 | 60/223,416 | Aug. 04, 2000 |
| SEQ ID NO:223 | R0262:E03 | 60/223,416 | Aug. 04, 2000 |
| SEQ ID NO:224 | R0262:F06 | 60/223,416 | Aug. 04, 2000 |
| SEQ ID NO:225 | R0263:B03 | 60/223,416 | Aug. 04, 2000 |
| SEQ ID NO:226 | R0263:B09 | 60/223,416 | Aug. 04, 2000 |
| SEQ ID NO:227 | R0263:E03 | 60/223,416 | Aug. 04, 2000 |
| SEQ ID NO:228 | R0263:F08 | 60/223,416 | Aug. 04, 2000 |
| SEQ ID NO:229 | R0263:G10 | 60/223,416 | Aug. 04, 2000 |
| SEQ ID NO:230 | R0263:G02 | 60/223,416 | Aug. 04, 2000 |
| SEQ ID NO:231 | R0263:G03 | 60/223,416 | Aug. 04, 2000 |
| SEQ ID NO:232 | R0263:H10 | 60/223,416 | Aug. 04, 2000 |
| SEQ ID NO:233 | R0264:A02 | 60/223,416 | Aug. 04, 2000 |
| SEQ ID NO:234 | R0264:B11 | 60/223,416 | Aug. 04, 2000 |
| SEQ ID NO:235 | R0264:E12 | 60/223,416 | Aug. 04, 2000 |
| SEQ ID NO:236 | R0264:F11 | 60/223,416 | Aug. 04, 2000 |
| SEQ ID NO:237 | R0264:F09 | 60/223,416 | Aug. 04, 2000 |
| SEQ ID NO:238 | R0264:G01 | 60/223,416 | Aug. 04, 2000 |
| SEQ ID NO:239 | R0264:G11 | 60/223,416 | Aug. 04, 2000 |
| SEQ ID NO:240 | R0264:G04 | 60/223,416 | Aug. 04, 2000 |
| SEQ ID NO:241 | R0265:F07 | 60/223,416 | Aug. 04, 2000 |
| SEQ ID NO:242 | R0265:G01 | 60/223,416 | Aug. 04, 2000 |
| SEQ ID NO:243 | R0265:G10 | 60/223,416 | Aug. 04, 2000 |
| SEQ ID NO:244 | R0265:G11 | 60/223,416 | Aug. 04, 2000 |
| SEQ ID NO:245 | R0265:H09 | 60/223,416 | Aug. 04, 2000 |
| SEQ ID NO:246 | R0266:A11 | 60/223,416 | Aug. 04, 2000 |
| SEQ ID NO:247 | R0266:A12 | 60/223,416 | Aug. 04, 2000 |
| SEQ ID NO:248 | R0266:B01 | 60/223,416 | Aug. 04, 2000 |
| SEQ ID NO:249 | R0266:C12 | 60/223,416 | Aug. 04, 2000 |
| SEQ ID NO:250 | R0266:E01 | 60/223,416 | Aug. 04, 2000 |
| SEQ ID NO:251 | R0266:E03 | 60/223,416 | Aug. 04, 2000 |
| SEQ ID NO:252 | R0266:F03 | 60/223,416 | Aug. 04, 2000 |
| SEQ ID NO:253 | R0266:F07 | 60/223,416 | Aug. 04, 2000 |
| SEQ ID NO:254 | R0266:G10 | 60/223,416 | Aug. 04, 2000 |
| SEQ ID NO:255 | R0266:G09 | 60/223,416 | Aug. 04, 2000 |
| SEQ ID NO:256 | R0266:H09 | 60/223,416 | Aug. 04, 2000 |
| SEQ ID NO:257 | R0243:F07 | 60/223,416 | Aug. 04, 2000 |
| SEQ ID NO:258 | R0244:C02 | 60/223,416 | Aug. 04, 2000 |
| SEQ ID NO:259 | R0244:C04 | 60/223,416 | Aug. 04, 2000 |
| SEQ ID NO:260 | R0245:A02 | 60/223,416 | Aug. 04, 2000 |
| SEQ ID NO:261 | ′46802.1_gaiger.ABI′ | 60/200,545 | Apr. 27, 2000 |
| SEQ ID NO:262 | ′46816.1_gaiger.ABI′ | 60/200,545 | Apr. 27, 2000 |
| SEQ ID NO:263 | ′46880.1_gaiger.ABI′ | 60/200,545 | Apr. 27, 2000 |
| SEQ ID NO:264 | ′47011.1_gaiger.ABI′ | 60/200,545 | Apr. 27, 2000 |
| SEQ ID NO:265 | ′51658.1_gaiger.ABI′ | 60/206,201 | May. 22, 2000 |
| SEQ ID NO:266 | ′51713.1_gaiger.ABI′ | 60/206,201 | May. 22, 2000 |
| SEQ ID NO:267 | ′51731.1_gaiger.ABI′ | 60/206,201 | May. 22, 2000 |
| SEQ ID NO:268 | ′51734.1_gaiger.ABI′ | 60/206,201 | May. 22, 2000 |
| SEQ ID NO:269 | ′51735.1_gaiger.ABI′ | 60/206,201 | May. 22, 2000 |
| SEQ ID NO:270 | ′51788.1_gaiger.ABI′ | 60/206,201 | May. 22, 2000 |
| SEQ ID NO:271 | ′51892.1_gaiger.ABI′ | 60/206,201 | May. 22, 2000 |
| SEQ ID NO:272 | ′51900.1_gaiger.ABI′ | 60/206,201 | May. 22, 2000 |
| SEQ ID NO:273 | ′51903.1_gaiger.ABI′ | 60/206,201 | May. 22, 2000 |
| SEQ ID NO:274 | 1404:D07 | 60/218,950 | Jul. 14, 2000 |
| SEQ ID NO:275 | 1405:C04 | 60/218,950 | Jul. 14, 2000 |
| SEQ ID NO:276 | 1405:D12 | 60/218,950 | Jul. 14, 2000 |
| SEQ ID NO:277 | 1405:E11 | 60/218,950 | Jul. 14, 2000 |
| SEQ ID NO:278 | ′52333.1_gaiger.ABI′ | 60/206,201 | May. 22, 2000 |
| SEQ ID NO:279 | ′41557.1_gaiger.ABI′ | 60/190,479 | Mar. 17, 2000 |
| SEQ ID NO:280 | ′41579.1_gaiger.ABI′ | 60/190,479 | Mar. 17, 2000 |
| SEQ ID NO:281 | ′41571.1_gaiger.ABI′ | 60/190,479 | Mar. 17, 2000 |
| SEQ ID NO:282 | ′41613.1_gaiger.ABI′ | 60/190,479 | Mar. 17, 2000 |
| SEQ ID NO:283 | ′41650.1_gaiger.ABI′ | 60/190,479 | Mar. 17, 2000 |
| SEQ ID NO:284 | ′41663.1_gaiger.ABI′ | 60/190,479 | Mar. 17, 2000 |
| SEQ ID NO:285 | ′41659.1_gaiger.ABI′ | 60/190,479 | Mar. 17, 2000 |
| SEQ ID NO:286 | ′41687.1_gaiger.ABI′ | 60/190,479 | Mar. 17, 2000 |
| SEQ ID NO:287 | ′41717.1_gaiger.ABI′ | 60/190,479 | Mar. 17, 2000 |
| SEQ ID NO:288 | ′41751.1_gaiger.ABI′ | 60/190,479 | Mar. 17, 2000 |
| SEQ ID NO:289 | ′41818.1_gaiger.ABI′ | 60/190,479 | Mar. 17, 2000 |
| SEQ ID NO:290 | ′41828.1_gaiger.ABI′ | 60/190,479 | Mar. 17, 2000 |
| SEQ ID NO:291 | ′41849.1_gaiger.ABI′ | 60/190,479 | Mar. 17, 2000 |
| SEQ ID NO:292 | ′41881.1_gaiger.ABI′ | 60/190,479 | Mar. 17, 2000 |
| SEQ ID NO:293 | ′41912.1_gaiger.ABI′ | 60/190,479 | Mar. 17, 2000 |
| SEQ ID NO:294 | ′41927.1_gaiger.ABI′ | 60/190,479 | Mar. 17, 2000 |
| SEQ ID NO:295 | ′41929.1_gaiger.ABI′ | 60/190,479 | Mar. 17, 2000 |
| SEQ ID NO:296 | ′41944.1_gaiger.ABI′ | 60/190,479 | Mar. 17, 2000 |
| SEQ ID NO:297 | ′41987.1_gaiger.ABI′ | 60/190,479 | Mar. 17, 2000 |
| SEQ ID NO:298 | ′41995.1_gaiger.ABI′ | 60/190,479 | Mar. 17, 2000 |
| SEQ ID NO:299 | ′42012.1_gaiger.ABI′ | 60/190,479 | Mar. 17, 2000 |
| SEQ ID NO:300 | ′42039.1_gaiger.ABI′ | 60/190,479 | Mar. 17, 2000 |
| SEQ ID NO:301 | ′42097.1_gaiger.ABI′ | 60/190,479 | Mar. 17, 2000 |
| SEQ ID NO:302 | ′42103.1_gaiger.ABI′ | 60/190,479 | Mar. 17, 2000 |
| SEQ ID NO:303 | ′42108.1_gaiger.ABI′ | 60/190,479 | Mar. 17, 2000 |
| SEQ ID NO:304 | R0233:A06 | 60/206,201 | May. 22, 2000 |
| SEQ ID NO:305 | R0233:A08 | 60/206,201 | May. 22, 2000 |
| SEQ ID NO:306 | R0233:C02 | 60/206,201 | May. 22, 2000 |
| SEQ ID NO:307 | R0233:E06 | 60/206,201 | May. 22, 2000 |
| SEQ ID NO:308 | R0233:F08 | 60/206,201 | May. 22, 2000 |
| SEQ ID NO:309 | ′42324.1_gaiger.ABI′ | 60/200,779 | May. 22, 2000 |
| SEQ ID NO:310 | ′42335.1_gaiger.ABI′ | 60/200,779 | May. 22, 2000 |
| SEQ ID NO:311 | ′42325.1_gaiger.ABI′ | 60/200,779 | May. 22, 2000 |
| SEQ ID NO:312 | ′42401.1_gaiger.ABI′ | 60/200,779 | May. 22, 2000 |
| SEQ ID NO:313 | ′42469.1;gaiger.ABI′ | 60/200,779 | May. 22, 2000 |
| SEQ ID NO:314 | ′42514.1;gaiger.ABI′ | 60/200,779 | May. 22, 2000 |
| SEQ ID NO:315 | ′42554.1;gaiger.ABI′ | 60/200,779 | May. 22, 2000 |
| SEQ ID NO:316 | ′42560.1;gaiger.ABI′ | 60/200,779 | May. 22, 2000 |
| SEQ ID NO:317 | ′42588.1_gaiger.ABI′ | 60/200,779 | May. 22, 2000 |
| SEQ ID NO:318 | ′42595.1_gaiger.ABI′ | 60/200,779 | May 22, 2000 |
| SEQ ID NO:319 | ′42609.1_gaiger.ABI′ | 60/200,779 | May 22, 2000 |
| SEQ ID NO:320 | ′42703.1_gaiger.ABI′ | 60/200,779 | May 22, 2000 |
| SEQ ID NO:321 | R0234:E06 | 60/206,201 | May 22, 2000 |
| SEQ ID NO:322 | R0234:F09 | 60/206,201 | May 22, 2000 |
| SEQ ID NO:323 | R0235:A09 | 60/206,201 | May 22, 2000 |
| SEQ ID NO:324 | R0235:D01 | 60/206,201 | May 22, 2000 |
| SEQ ID NO:325 | R0236:D04 | 60/206,201 | May 22, 2000 |
| SEQ ID NO:326 | R0236:F10 | 60/206,201 | May 22, 2000 |
| SEQ ID NO:327 | R0236:G10 | 60/206,201 | May 22, 2000 |
| SEQ ID NO:328 | R0236:G08 | 60/206,201 | May 22, 2000 |
| SEQ ID NO:329 | R0249:D01 | 60/222,903 | Aug. 03, 2000 |
| SEQ ID NO:330 | R0249:G04 | 60/222,903 | Aug. 03, 2000 |
| SEQ ID NO:331 | R0250:A10 | 60/222,903 | Aug. 03, 2000 |
| SEQ ID NO:332 | R0250:E12 | 60/222,903 | Aug. 03, 2000 |
| SEQ ID NO:333 | R0250:F12 | 60/222,903 | Aug. 03, 2000 |
| SEQ ID NO:334 | R0251:B08 | 60/222,903 | Aug. 03, 2000 |
| SEQ ID NO:335 | R0252:A08 | 60/222,903 | Aug. 03, 2000 |
| SEQ ID NO:336 | R0252:F11 | 60/222,903 | Aug. 03, 2000 |
| SEQ ID NO:337 | R0252:F02 | 60/222,903 | Aug. 03, 2000 |
| SEQ ID NO:338 | R0252:F08 | 60/222,903 | Aug. 03, 2000 |
| SEQ ID NO:339 | R0252:G11 | 60/222,903 | Aug. 03, 2000 |
| SEQ ID NO:340 | R0253:E10 | 60/222,903 | Aug. 03, 2000 |
| SEQ ID NO:341 | R0253:G11 | 60/222,903 | Aug. 03, 2000 |
| SEQ ID NO:342 | R0254:A08 | 60/223,416 | Aug. 04, 2000 |
| SEQ ID NO:343 | R0254:E04 | 60/223,416 | Aug. 04, 2000 |
| SEQ ID NO:344 | R0254:F07 | 60/223,416 | Aug. 04, 2000 |
| SEQ ID NO:345 | R0237:F12 | 60/206,201 | May 22, 2000 |
| SEQ ID NO:346 | R0238:B02 | 60/223,416 | Aug. 04, 2000 |
| SEQ ID NO:347 | R0238:D06 | 60/223,416 | Aug. 04, 2000 |
| SEQ ID NO:348 | R0238:F03 | 60/223,416 | Aug. 04, 2000 |
| SEQ ID NO:349 | R0239:H02 | 60/206,201 | May 22, 2000 |
| SEQ ID NO:350 | R0255:F12 | 60/223,416 | Aug. 04, 2000 |
| SEQ ID NO:351 | R0258:B10 | 60/223,416 | Aug. 04, 2000 |
| SEQ ID NO:352 | R0259:C06 | 60/223,416 | Aug. 04, 2000 |
| SEQ ID NO:353 | R0261:A09 | 60/223,416 | Aug. 04, 2000 |
| SEQ ID NO:354 | R0261:B10 | 60/223,416 | Aug. 04, 2000 |
| SEQ ID NO:355 | R0261:C10 | 60/223,416 | Aug. 04, 2000 |
| SEQ ID NO:356 | R0261:D03 | 60/223,416 | Aug. 04, 2000 |
| SEQ ID NO:357 | R0261:D06 | 60/223,416 | Aug. 04, 2000 |
| SEQ ID NO:358 | R0261:E10 | 60/223,416 | Aug. 04, 2000 |
| SEQ ID NO:359 | R0261:F10 | 60/223,416 | Aug. 04, 2000 |
| SEQ ID NO:360 | R0261:G04 | 60/223,416 | Aug. 04, 2000 |
| SEQ ID NO:361 | R0262:A12 | 60/223,416 | Aug. 04, 2000 |
| SEQ ID NO:362 | R0262:A03 | 60/223,416 | Aug. 04, 2000 |
| SEQ ID NO:363 | R0262:B09 | 60/223,416 | Aug. 04, 2000 |
| SEQ ID NO:364 | R0262:C04 | 60/223,416 | Aug. 04, 2000 |
| SEQ ID NO:365 | R0262:D11 | 60/223,416 | Aug. 04, 2000 |
| SEQ ID NO:366 | R0262:D12 | 60/223,416 | Aug. 04, 2000 |
| SEQ ID NO:367 | R0262:D04 | 60/223,416 | Aug. 04, 2000 |
| SEQ ID NO:368 | R0262:D07 | 60/223,416 | Aug. 04, 2000 |
| SEQ ID NO:369 | R0262:E02 | 60/223,416 | Aug. 04, 2000 |
| SEQ ID NO:370 | R0262:G05 | 60/223,416 | Aug. 04, 2000 |
| SEQ ID NO:371 | R0263:B10 | 60/223,416 | Aug. 04, 2000 |
| SEQ ID NO:372 | R0263:B06 | 60/223,416 | Aug. 04, 2000 |
| SEQ ID NO:373 | R0263:B09 | 60/223,416 | Aug. 04, 2000 |
| SEQ ID NO:374 | R0263:D11 | 60/223,416 | Aug. 04, 2000 |
| SEQ ID NO:375 | R0263:D07 | 60/223,416 | Aug. 04, 2000 |
| SEQ ID NO:376 | R0263:E03 | 60/223,416 | Aug. 04, 2000 |
| SEQ ID NO:377 | R0263:F08 | 60/223,416 | Aug. 04, 2000 |
| SEQ ID NO:378 | R0263:G03 | 60/223,416 | Aug. 04, 2000 |
| SEQ ID NO:379 | R0263:H10 | 60/223,416 | Aug. 04, 2000 |
| SEQ ID NO:380 | R0263:H02 | 60/223,416 | Aug. 04, 2000 |
| SEQ ID NO:381 | R0264:B11 | 60/223,416 | Aug. 04, 2000 |
| SEQ ID NO:382 | R0264:D03 | 60/223,416 | Aug. 04, 2000 |
| SEQ ID NO:383 | R0264:E12 | 60/223,416 | Aug. 04, 2000 |
| SEQ ID NO:384 | R0264:F11 | 60/223,416 | Aug. 04, 2000 |
| SEQ ID NO:385 | R0264:F09 | 60/223,416 | Aug. 04, 2000 |
| SEQ ID NO:386 | R0264:G03 | 60/223,416 | Aug. 04, 2000 |
| SEQ ID NO:387 | R0264:G04 | 60/223,416 | Aug. 04, 2000 |
| SEQ ID NO:388 | R0264:G06 | 60/223,416 | Aug. 04, 2000 |
| SEQ ID NO:389 | R0264:G09 | 60/223,416 | Aug. 04, 2000 |
| SEQ ID NO:390 | R0264:H04 | 60/223,416 | Aug. 04, 2000 |
| SEQ ID NO:391 | R0265:A09 | 60/223,416 | Aug. 04, 2000 |
| SEQ ID NO:392 | R0265:D10 | 60/223,416 | Aug. 04, 2000 |
| SEQ ID NO:393 | R0265:D07 | 60/223,416 | Aug. 04, 2000 |
| SEQ ID NO:394 | R0265:E12 | 60/223,416 | Aug. 04, 2000 |
| SEQ ID NO:395 | R0265:F12 | 60/223,416 | Aug. 04, 2000 |
| SEQ ID NO:396 | R0265:H04 | 60/223,416 | Aug. 04, 2000 |
| SEQ ID NO:397 | R0265:H09 | 60/223,416 | Aug. 04, 2000 |
| SEQ ID NO:398 | R0266:A10 | 60/223,416 | Aug. 04, 2000 |
| SEQ ID NO:399 | R0266:A12 | 60/223,416 | Aug. 04, 2000 |
| SEQ ID NO:400 | R0266:B02 | 60/223,416 | Aug. 04, 2000 |
| SEQ ID NO:401 | R0266:C12 | 60/223,416 | Aug. 04, 2000 |
| SEQ ID NO:402 | R0266:E08 | 60/223,416 | Aug. 04, 2000 |
| SEQ ID NO:403 | R0266:F03 | 60/223,416 | Aug. 04, 2000 |
| SEQ ID NO:404 | R0266:F06 | 60/223,416 | Aug. 04, 2000 |
| SEQ ID NO:405 | R0266:F07 | 60/223,416 | Aug. 04, 2000 |
| SEQ ID NO:406 | R0266:G12 | 60/223,416 | Aug. 04, 2000 |
| SEQ ID NO:407 | R0266:G09 | 60/223,416 | Aug. 04, 2000 |
| SEQ ID NO:408 | R0266:H06 | 60/223,416 | Aug. 04, 2000 |
| SEQ ID NO:409 | R0242:E03 | 60/223,416 | Aug. 04, 2000 |
| SEQ ID NO:410 | R0244:C04 | 60/223,416 | Aug. 04, 2000 |
| SEQ ID NO:411 | R0244:C06 | 60/223,416 | Aug. 04, 2000 |
| SEQ ID NO:412 | R0245:A02 | 60/223,416 | Aug. 04, 2000 |
| SEQ ID NO:413 | R0245:D12 | 60/223,416 | Aug. 04, 2000 |
| SEQ ID NO:414 | R0246:D10 | 60/223,416 | Aug. 04, 2000 |
| SEQ ID NO:415 | ′46377.1_gaiger.ABI′ | 60/200,545 | Apr. 27, 2000 |
| SEQ ID NO:416 | ′46403.1_gaiger.ABI′ | 60/200,545 | Apr. 27, 2000 |
| SEQ ID NO:417 | ′46489.1;gaiger.ABI′ | 60/200,545 | Apr. 27, 2000 |
| SEQ ID NO:418 | ′46872.1_gaiger.ABI′ | 60/200,545 | Apr. 27, 2000 |
| SEQ ID NO:419 | ′46883.1_gaiger.ABI′ | 60/200,545 | Apr. 27, 2000 |
| SEQ ID NO:420 | ′46880.1_gaiger.ABI′ | 60/200,545 | Apr. 27, 2000 |
| SEQ ID NO:421 | ′46977.1_gaiger.ABI′ | 60/200,545 | Apr. 27, 2000 |
| SEQ ID NO:422 | ′47011.1_gaiger.ABI′ | 60/200,545 | Apr. 27, 2000 |
| SEQ ID NO:423 | ′51658.1_gaiger.ABI′ | 60/206,201 | May 22, 2000 |
| SEQ ID NO:424 | ′51713.1_gaiger.ABI′ | 60/206,201 | May 22, 2000 |
| SEQ ID NO:425 | ′51734.1_gaiger.ABI′ | 60/206,201 | May 22, 2000 |
| SEQ ID NO:426 | ′51766.1_gaiger.ABI′ | 60/206,201 | May 22, 2000 |
| SEQ ID NO:427 | ′51870.1_gaiger.ABI′ | 60/206,201 | May 22, 2000 |
| SEQ ID NO:428 | ′51924.1_gaiger.ABI′ | 60/206,201 | May 22, 2000 |
| SEQ ID NO:429 | 1404:A06 | 60/218,950 | Jul. 14, 2000 |
| SEQ ID NO:430 | 1404:B12 | 60/218,950 | Jul. 14, 2000 |
| SEQ ID NO:431 | 1404:D12 | 60/218,950 | Jul. 14, 2000 |
| SEQ ID NO:432 | 1404:E11 | 60/218,950 | Jul. 14, 2000 |
| SEQ ID NO:433 | 1405:A11 | 60/218,950 | Jul. 14, 2000 |
| SEQ ID NO:434 | ′52280.1_gaiger.ABI′ | 60/206,201 | May 22, 2000 |
| SEQ ID NO:435 | ′52345.1_gaiger.ABI′ | 60/206,201 | May 22, 2000 |
| SEQ ID NO:436 | ′52373.1_gaiger.ABI′ | 60/206,20 1 | May 22, 2000 |
| SEQ ID NO:437 | R0238:F03 | 60/223,416 | Aug. 04, 2000 |
| SEQ ID NO:438 | R0263:E03 | 60/223,416 | Aug. 04, 2000 |
| SEQ ID NO:439 | ′41557.1_gaiger.ABI′ | 60/190,479 | Mar. 17, 2000 |
| SEQ ID NO:440 | ′41650.1_gaiger.ABI′ | 60/190,479 | Mar. 17, 2000 |
| SEQ ID NO:441 | ′41663.1_gaiger.ABI′ | 60/190,479 | Mar. 17, 2000 |
| SEQ ID NO:442 | ′41659.1_gaiger.ABI′ | 60/190,479 | Mar. 17, 2000 |
| SEQ ID NO:443 | ′41667.1_gaiger.ABI′ | 60/190,479 | Mar. 17, 2000 |
| SEQ ID NO:444 | ′41729.1_gaiger.ABI′ | 60/190,479 | Mar. 17, 2000 |
| SEQ ID NO:445 | ′41751.1_gaiger.ABI′ | 60/190,479 | Mar. 17, 2000 |
| SEQ ID NO:446 | ′41818.1_gaiger.ABI′ | 60/190,479 | Mar. 17, 2000 |
| SEQ ID NO:447 | ′41828.1_gaiger.ABI′ | 60/190,479 | Mar. 17, 2000 |
| SEQ ID NO:448 | ′41847.1_gaiger.ABI′ | 60/190,479 | Mar. 17, 2000 |
| SEQ ID NO:449 | ′41849.1_gaiger.ABI′ | 60/190,479 | Mar. 17, 2000 |
| SEQ ID NO:450 | ′41927.1_gaiger.ABI′ | 60/190,479 | Mar. 17, 2000 |
| SEQ ID NO:451 | ′41929.1_gaiger.ABI′ | 60/190,479 | Mar. 17, 2000 |
| SEQ ID NO:452 | ′41995.1_gaiger.ABI′ | 60/190,479 | Mar. 17, 2000 |
| SEQ ID NO:453 | ′42012.1_gaiger.ABI′ | 60/190,479 | Mar. 17, 2000 |
| SEQ ID NO:454 | ′42039.1_gaiger.ABI′ | 60/190,479 | Mar. 17, 2000 |
| SEQ ID NO:455 | ′42097.1_gaiger.ABI′ | 60/190,479 | Mar. 17, 2000 |
| SEQ ID NO:456 | ′42108.1_gaiger.ABI′ | 60/190,479 | Mar. 17, 2000 |
| SEQ ID NO:457 | R0233:A06 | 60/206,201 | May 22, 2000 |
| SEQ ID NO:458 | R0233:C02 | 60/206,201 | May 22, 2000 |
| SEQ ID NO:459 | R0233:E06 | 60/206,201 | May 22, 2000 |
| SEQ ID NO:460 | R0233:F08 | 60/206,201 | May 22, 2000 |
| SEQ ID NO:461 | ′42325.1_gaiger.ABI′ | 60/200,779 | May 22, 2000 |
| SEQ ID NO:462 | ′42328.1_gaiger.ABI′ | 60/200,779 | May 22, 2000 |
| SEQ ID NO:463 | ′42401.1_gaiger.ABI′ | 60/200,779 | May 22, 2000 |
| SEQ ID NO:464 | ′42588.1_gaiger.ABI′ | 60/200,779 | May 22, 2000 |
| SEQ ID NO:465 | ′42595.1_gaiger.ABI′ | 60/200,779 | May 22, 2000 |
| SEQ ID NO:466 | ′42703.1_gaiger.ABI′ | 60/200,779 | May 22, 2000 |
| SEQ ID NO:467 | R0234:B07 | 60/206,201 | May 22, 2000 |
| SEQ ID NO:468 | R0234:E06 | 60/206,201 | May 22, 2000 |
| SEQ ID NO:469 | R0234:F09 | 60/206,201 | May 22, 2000 |
| SEQ ID NO:470 | R0235:B03 | 60/206,201 | May 22, 2000 |
| SEQ ID NO:471 | R0235:E05 | 60/206,201 | May 22, 2000 |
| SEQ ID NO:472 | R0236:A06 | 60/206,201 | May 22, 2000 |
| SEQ ID NO:473 | R0236:D04 | 60/206,201 | May 22, 2000 |
| SEQ ID NO:474 | R0250:A10 | 60/222,903 | Aug. 03, 2000 |
| SEQ ID NO:475 | R0251:E09 | 60/222,903 | Aug. 03, 2000 |
| SEQ ID NO:476 | R0252:F11 | 60/222,903 | Aug. 03, 2000 |
| SEQ ID NO:477 | R0238:B02 | 60/223,416 | Aug. 04, 2000 |
| SEQ ID NO:478 | R0239:H02 | 60/206,201 | May 22, 2000 |
| SEQ ID NO:479 | R0255:F12 | 60/223,416 | Aug. 04, 2000 |
| SEQ ID NO:480 | R0259:C06 | 60/223,416 | Aug. 04, 2000 |
| SEQ ID NO:481 | R0261:B10 | 60/223,416 | Aug. 04, 2000 |
| SEQ ID NO:482 | R0261:D06 | 60/223,416 | Aug. 04, 2000 |
| SEQ ID NO:483 | R0261:E10 | 60/223,416 | Aug. 04, 2000 |
| SEQ ID NO:484 | R0261:H08 | 60/223,416 | Aug. 04, 2000 |
| SEQ ID NO:485 | R0262:A12 | 60/223,416 | Aug. 04, 2000 |
| SEQ ID NO:486 | R0262:A03 | 60/223,416 | Aug. 04, 2000 |
| SEQ ID NO:487 | R0262:D11 | 60/223,416 | Aug. 04, 2000 |
| SEQ ID NO:488 | R0262:E03 | 60/223,416 | Aug. 04, 2000 |
| SEQ ID NO:489 | R0262:G05 | 60/223,416 | Aug. 04, 2000 |
| SEQ ID NO:490 | R0263:B11 | 60/223,416 | Aug. 04, 2000 |
| SEQ ID NO:491 | R0263:D11 | 60/223,416 | Aug. 04, 2000 |
| SEQ ID NO:492 | R0263:D07 | 60/223,416 | Aug. 04, 2000 |
| SEQ ID NO:493 | R0263:F08 | 60/223,416 | Aug. 04, 2000 |
| SEQ ID NO:494 | R0263:H02 | 60/223,416 | Aug. 04, 2000 |
| SEQ ID NO:495 | R0264:D03 | 60/223,416 | Aug. 04, 2000 |
| SEQ ID NO:496 | R0264:E12 | 60/223,416 | Aug. 04, 2000 |
| SEQ ID NO:497 | R0264:F11 | 60/223,416 | Aug. 04, 2000 |
| SEQ ID NO:498 | R0264:H03 | 60/223,416 | Aug. 04, 2000 |
| SEQ ID NO:499 | R0265:D07 | 60/223,416 | Aug. 04, 2000 |
| SEQ ID NO:500 | R0265:E12 | 60/223,416 | Aug. 04, 2000 |
| SEQ ID NO:501 | R0265:F12 | 60/223,416 | Aug. 04, 2000 |
| SEQ ID NO:502 | R0265:H04 | 60/223,416 | Aug. 04, 2000 |
| SEQ ID NO:503 | R0265:H09 | 60/223,416 | Aug. 04, 2000 |
| SEQ ID NO:504 | R0266:A10 | 60/223,416 | Aug. 04, 2000 |
| SEQ ID NO:505 | R0266:A12 | 60/223,416 | Aug. 04, 2000 |
| SEQ ID NO:506 | R0266:F03 | 60/223,416 | Aug. 04, 2000 |
| SEQ ID NO:507 | R0266:F07 | 60/223,416 | Aug. 04, 2000 |
| SEQ ID NO:508 | R0266:G12 | 60/223,416 | Aug. 04, 2000 |
| SEQ ID NO:509 | R0266:G09 | 60/223,416 | Aug. 04, 2000 |
| SEQ ID NO:510 | R0266:H06 | 60/223,416 | Aug. 04, 2000 |
| SEQ ID NO:511 | R0244:C04 | 60/223,416 | Aug. 04, 2000 |
| SEQ ID NO:512 | R0245:A02 | 60/223,416 | Aug. 04, 2000 |
| SEQ ID NO:513 | R0246:D10 | 60/223,416 | Aug. 04, 2000 |
| SEQ ID NO:514 | ′46403.1_gaiger.ABI′ | 60/200,545 | Apr. 27, 2000 |
| SEQ ID NO:515 | ′46458.1_gaiger.ABI′ | 60/200,545 | Apr. 27, 2000 |
| SEQ ID NO:516 | ′46489.1;gaiger.ABI′ | 60/200,545 | Apr. 27, 2000 |
| SEQ ID NO:517 | ′46802.1_gaiger.ABI′ | 60/200,545 | Apr. 27, 2000 |
| SEQ ID NO:518 | ′46872.1_gaiger.ABI′ | 60/200,545 | Apr. 27, 2000 |
| SEQ ID NO:519 | ′46880.1_gaiger.ABI′ | 60/200,545 | Apr. 27, 2000 |
| SEQ ID NO:520 | ′46977.1_gaiger.ABI′ | 60/200,545 | Apr. 27, 2000 |
| SEQ ID NO:521 | ′51658.1_gaiger.ABI′ | 60/206,201 | May 22, 2000 |
| SEQ ID NO:522 | ′51713.1_gaiger.ABI′ | 60/206,201 | May 22, 2000 |
| SEQ ID NO:523 | ′51734.1_gaiger.ABI′ | 60/206,201 | May 22, 2000 |
| SEQ ID NO:524 | ′51924.1_gaiger.ABI′ | 60/206,201 | May 22, 2000 |
| SEQ ID NO:525 | 1405:C04 | 60/218,950 | Jul. 14, 2000 |
| SEQ ID NO:526 | 1405:E11 | 60/218,950 | Jul. 14, 2000 |
| SEQ ID NO:527 | ′52246.1_gaiger.ABI′ | 60/206,201 | May 22, 2000 |
| SEQ ID NO:528 | ′52333.1_gaiger.ABI′ | 60/206,201 | May 22, 2000 |
| SEQ ID NO:529 | ′41557.1_gaiger.ABI′ | 60/190,479 | Mar. 17, 2000 |
| SEQ ID NO:530 | ′41579.1_gaiger.ABI′ | 60/190,479 | Mar. 17, 2000 |
| SEQ ID NO:531 | ′41571.1_gaiger.ABI′ | 60/190,479 | Mar. 17, 2000 |
| SEQ ID NO:532 | ′41573.1_gaiger.ABI′ | 60/190,479 | Mar. 17, 2000 |
| SEQ ID NO:533 | ′41628.1_gaiger.ABI′ | 60/190,479 | Mar. 17, 2000 |
| SEQ ID NO:534 | ′41635.1_gaiger.ABI′ | 60/190,479 | Mar. 17, 2000 |
| SEQ ID NO:535 | ′41663.1_gaiger.ABI′ | 60/190,479 | Mar. 17, 2000 |
| SEQ ID NO:536 | ′41667.1_gaiger.ABI′ | 60/190,479 | Mar. 17, 2000 |
| SEQ ID NO:537 | ′41751.1_gaiger.ABI′ | 60/190,479 | Mar. 17, 2000 |
| SEQ ID NO:538 | ′41944.1_gaiger.ABI′ | 60/190,479 | Mar. 17, 2000 |
| SEQ ID NO:539 | ′41986.1_gaiger.ABI′ | 60/190,479 | Mar. 17, 2000 |
| SEQ ID NO:540 | ′42101.1_gaiger.ABI′ | 60/190,479 | Mar. 17, 2000 |
| SEQ ID NO:541 | R0232:E07 | 60/206,201 | May 22, 2000 |
| SEQ ID NO:542 | R0233:A06 | 60/206,201 | May 22, 2000 |
| SEQ ID NO:543 | ′42324.1_gaiger.ABI′ | 60/200,779 | May 22, 2000 |
| SEQ ID NO:544 | ′42438.1_gaiger.ABI′ | 60/200,779 | May 22, 2000 |
| SEQ ID NO:545 | ′42625.1_gaiger.ABI′ | 60/200,779 | May 22, 2000 |
| SEQ ID NO:546 | ′42702.1_gaiger.ABI′ | 60/200,779 | May 22, 2000 |
| SEQ ID NO:547 | ′42709.1_gaiger.ABI′ | 60/200,779 | May 22, 2000 |
| SEQ ID NO:548 | R0234:E07 | 60/206,201 | May 22, 2000 |
| SEQ ID NO:549 | R0234:G11 | 60/206,201 | May 22, 2000 |
| SEQ ID NO:550 | R0236:A09 | 60/206,201 | May 22, 2000 |
| SEQ ID NO:551 | R0250:A05 | 60/222,903 | Aug. 03, 2000 |
| SEQ ID NO:552 | R0251:A07 | 60/222,903 | Aug. 03, 2000 |
| SEQ ID NO:553 | R0251:D01 | 60/222,903 | Aug. 03, 2000 |
| SEQ ID NO:554 | R0252:A08 | 60/222,903 | Aug. 03, 2000 |
| SEQ ID NO:555 | R0252:F11 | 60/222,903 | Aug. 03, 2000 |
| SEQ ID NO:556 | R0252:H01 | 60/222,903 | Aug. 03, 2000 |
| SEQ ID NO:557 | R0253:E09 | 60/222,903 | Aug. 03, 2000 |
| SEQ ID NO:558 | R0253:G05 | 60/222,903 | Aug. 03, 2000 |
| SEQ ID NO:559 | R0253:G06 | 60/222,903 | Aug. 03, 2000 |
| SEQ ID NO:560 | R0254:F07 | 60/223,416 | Aug. 04, 2000 |
| SEQ ID NO:561 | R0238:D06 | 60/223,416 | Aug. 04, 2000 |
| SEQ ID NO:562 | R0255:F12 | 60/223,416 | Aug. 04, 2000 |
| SEQ ID NO:563 | R0259:C04 | 60/223,416 | Aug. 04, 2000 |
| SEQ ID NO:564 | R0261:A09 | 60/223,416 | Aug. 04, 2000 |
| SEQ ID NO:565 | R0261:C10 | 60/223,416 | Aug. 04, 2000 |
| SEQ ID NO:566 | R0261:D06 | 60/223,416 | Aug. 04, 2000 |
| SEQ ID NO:567 | R0262:D04 | 60/223,416 | Aug. 04, 2000 |
| SEQ ID NO:568 | R0262:E03 | 60/223,416 | Aug. 04, 2000 |
| SEQ ID NO:569 | R0263:B11 | 60/223,416 | Aug. 04, 2000 |
| SEQ ID NO:570 | R0263:B09 | 60/223,416 | Aug. 04, 2000 |
| SEQ ID NO:571 | R0263:C08 | 60/223,416 | Aug. 04, 2000 |
| SEQ ID NO:572 | R0263:D11 | 60/223,416 | Aug. 04, 2000 |
| SEQ ID NO:573 | R0263:H10 | 60/223,416 | Aug. 04, 2000 |
| SEQ ID NO:574 | R0264:A03 | 60/223,416 | Aug. 04, 2000 |
| SEQ ID NO:575 | R0264:B11 | 60/223,416 | Aug. 04, 2000 |
| SEQ ID NO:576 | R0264:F11 | 60/223,416 | Aug. 04, 2000 |
| SEQ ID NO:577 | R0264:F05 | 60/223,416 | Aug. 04, 2000 |
| SEQ ID NO:578 | R0264:F09 | 60/223,416 | Aug. 04, 2000 |
| SEQ ID NO:579 | R0266:B02 | 60/223,416 | Aug. 04, 2000 |
| SEQ ID NO:580 | R0266:B03 | 60/223,416 | Aug. 04, 2000 |
| SEQ ID NO:581 | R0266:B04 | 60/223,416 | Aug. 04, 2000 |
| SEQ ID NO:582 | R0266:B06 | 60/223,416 | Aug. 04, 2000 |
| SEQ ID NO:583 | R0266:D05 | 60/223,416 | Aug. 04, 2000 |
| SEQ ID NO:584 | R0266:E01 | 60/223,416 | Aug. 04, 2000 |
| SEQ ID NO:585 | R0266:E03 | 60/223,416 | Aug. 04, 2000 |
| SEQ ID NO:586 | R0266:F03 | 60/223,416 | Aug. 04, 2000 |
| SEQ ID NO:587 | R0266:F09 | 60/223,416 | Aug. 04, 2000 |
| SEQ ID NO:588 | R0245:A02 | 60/223,416 | Aug. 04, 2000 |
| SEQ ID NO:589 | ′46403.1_gaiger.ABI′ | 60/200,545 | Apr. 27, 2000 |
| SEQ ID NO:590 | ′46458.1_gaiger.ABI′ | 60/200,545 | Apr. 27, 2000 |
| SEQ ID NO:591 | ′46977.1_gaiger.ABI′ | 60/200,545 | Apr. 27, 2000 |
| SEQ ID NO:592 | ′51658.1_gaiger.ABI′ | 60/206,201 | May 22, 2000 |
| SEQ ID NO:593 | ′51713.1_gaiger.ABI′ | 60/206,201 | May 22, 2000 |
| SEQ ID NO:594 | ′51731.1_gaiger.ABI′ | 60/206,201 | May 22, 2000 |
| SEQ ID NO:595 | ′51788.1_gaiger.ABI′ | 60/206,201 | May 22, 2000 |
| SEQ ID NO:596 | ′51850.1_gaiger.ABI′ | 60/206,201 | May 22, 2000 |
| SEQ ID NO:597 | ′51892.1_gaiger.ABI′ | 60/206,201 | May 22, 2000 |
| SEQ ID NO:598 | ′51900.1_gaiger.ABI′ | 60/206,201 | May 22, 2000 |
| SEQ ID NO:599 | ′51903.1_gaiger.ABI′ | 60/206,201 | May 22, 2000 |
| SEQ ID NO:600 | ′51960.1_gaiger.ABI′ | 60/206,201 | May 22, 2000 |
| SEQ ID NO:601 | 1405:A09 | 60/218,950 | Jul. 14, 2000 |
| SEQ ID NO:602 | 1405:D12 | 60/218,950 | Jul. 14, 2000 |
| SEQ ID NO:603 | 1405:D09 | 60/218,950 | Jul. 14, 2000 |
| SEQ ID NO:604 | 1405:E11 | 60/218,950 | Jul. 14, 2000 |
| SEQ ID NO:605 | ′52246.1_gaiger.ABI′ | 60/206,201 | May 22, 2000 |
| SEQ ID NO:606 | ′52333.1_gaiger.ABI′ | 60/206,201 | May 22, 2000 |
| SEQ ID NO:607 | 1408:A09 | 60/218,950 | Jul. 14, 2000 |
| SEQ ID NO:608 | 1408:B02 | 60/218,950 | Jul. 14, 2000 |
| SEQ ID NO:609 | 1408:C12 | 60/218,950 | Jul. 14, 2000 |
| SEQ ID NO:610 | 1408:D06 | 60/218,950 | Jul. 14, 2000 |
| SEQ ID NO:611 | ′41663.1_gaiger.ABI′ | 60/190,479 | Mar. 17, 2000 |
| SEQ ID NO:612 | ′41729.1_gaiger.ABI′ | 60/190,479 | Mar. 17, 2000 |
| SEQ ID NO:613 | ′41888.1_gaiger.ABI′ | 60/190,479 | Mar. 17, 2000 |
| SEQ ID NO:614 | ′41925.1_gaiger.ABI′ | 60/190,479 | Mar. 17, 2000 |
| SEQ ID NO:615 | ′41639.1_gaiger.ABI′ | 60/190,479 | Mar. 17, 2000 |
| SEQ ID NO:616 | ′41853.1_gaiger.ABI′ | 60/190,479 | Mar. 17, 2000 |
| SEQ ID NO:617 | ′41876.1_gaiger.ABI′ | 60/190,479 | Mar. 17, 2000 |
| SEQ ID NO:618 | ′41924.1_gaiger.ABI′ | 60/190,479 | Mar. 17, 2000 |
| SEQ ID NO:619 | ′41638.1_gaiger.ABI′ | 60/190,479 | Mar. 17, 2000 |
| SEQ ID NO:620 | ′41581.1_gaiger.ABI′ | 60/190,479 | Mar. 17, 2000 |
| SEQ ID NO:621 | ′41629.1_gaiger.ABI′ | 60/190,479 | Mar. 17, 2000 |
| SEQ ID NO:622 | ′41678.1_gaiger.ABI′ | 60/190,479 | Mar. 17, 2000 |
| SEQ ID NO:623 | ′41717.1_gaiger.ABI′ | 60/190,479 | Mar. 17, 2000 |
| SEQ ID NO:624 | ′41987.1_gaiger.ABI′ | 60/190,479 | Mar. 17, 2000 |
| SEQ ID NO:625 | R0233:F02 | 60/206,201 | May 22, 2000 |
| SEQ ID NO:626 | R0232:A08 | 60/206,201 | May 22, 2000 |
| SEQ ID NO:627 | R0233:B04 | 60/206,201 | May 22, 2000 |
| SEQ ID NO:628 | ′42041.1_gaiger.ABI′ | 60/190,479 | Mar. 17, 2000 |
| SEQ ID NO:629 | ′42387.1_gaiger.ABI′ | 60/200,779 | May 22, 2000 |
| SEQ ID NO:630 | ′42460.1;gaiger.ABI′ | 60/200,779 | May 22, 2000 |
| SEQ ID NO:631 | ′42407.1_gaiger.ABI′ | 60/200,779 | May 22, 2000 |
| SEQ ID NO:632 | ′42483.1;gaiger.ABI′ | 60/200,779 | May 22, 2000 |
| SEQ ID NO:633 | ′42350.1_gaiger.ABI′ | 60/200,779 | May 22, 2000 |
| SEQ ID NO:634 | ′42530.1;gaiger.ABI′ | 60/200,779 | May 22, 2000 |
| SEQ ID NO:635 | ′42523.1;gaiger.ABI′ | 60/200,779 | May 22, 2000 |
| SEQ ID NO:636 | R0235:D07 | 60/206,201 | May 22, 2000 |
| SEQ ID NO:637 | R0235:D12 | 60/206,201 | May 22, 2000 |
| SEQ ID NO:638 | R0236:H02 | 60/206,201 | May 22, 2000 |
| SEQ ID NO:639 | R0251:B12 | 60/222,903 | Aug. 03, 2000 |
| SEQ ID NO:640 | R0253:D09 | 60/222,903 | Aug. 03, 2000 |
| SEQ ID NO:641 | R0254:F10 | 60/223,416 | Aug. 04, 2000 |
| SEQ ID NO:642 | R0253:G01 | 60/222,903 | Aug. 03, 2000 |
| SEQ ID NO:643 | R0254:D02 | 60/223,416 | Aug. 04, 2000 |
| SEQ ID NO:644 | R0238:B06 | 60/223,416 | Aug. 04, 2000 |
| SEQ ID NO:645 | R0255:D01 | 60/223,416 | Aug. 04, 2000 |
| SEQ ID NO:646 | R0255:C02 | 60/223,416 | Aug. 04, 2000 |
| SEQ ID NO:647 | R0261:H04 | 60/223,416 | Aug. 04, 2000 |
| SEQ ID NO:648 | R0259:C04 | 60/223,416 | Aug. 04, 2000 |
| SEQ ID NO:649 | R0259:C06 | 60/223,416 | Aug. 04, 2000 |
| SEQ ID NO:650 | R0261:H08 | 60/223,416 | Aug. 04, 2000 |
| SEQ ID NO:651 | R0261:D03 | 60/223,416 | Aug. 04, 2000 |
| SEQ ID NO:652 | R0262:C04 | 60/223,416 | Aug. 04, 2000 |
| SEQ ID NO:653 | R0264:B08 | 60/223,416 | Aug. 04, 2000 |
| SEQ ID NO:654 | R0266:D03 | 60/223,416 | Aug. 04, 2000 |
| SEQ ID NO:655 | R0265:F12 | 60/223,416 | Aug. 04, 2000 |
| SEQ ID NO:656 | R0264:C03 | 60/223,416 | Aug. 04, 2000 |
| SEQ ID NO:657 | R0264:C04 | 60/223,416 | Aug. 04, 2000 |
| SEQ ID NO:658 | R0244:C02 | 60/223,416 | Aug. 04, 2000 |
| SEQ ID NO:659 | R0245:A02 | 60/223,416 | Aug. 04, 2000 |
| SEQ ID NO:660 | ′51734.1_gaiger.ABI′ | 60/206,201 | May 22, 2000 |
| SEQ ID NO:661 | ′51870.1_gaiger.ABI′ | 60/206,201 | May 22, 2000 |
| SEQ ID NO:662 | ′51791.1_gaiger.ABI′ | 60/206,201 | May 22, 2000 |
| SEQ ID NO:663 | ′51975.1_gaiger.ABI′ | 60/206,201 | May 22, 2000 |
| SEQ ID NO:664 | ′52260.1_gaiger.ABI′ | 60/206,201 | May 22, 2000 |
| SEQ ID NO:665 | TCL1 DNA | | |
| SEQ ID NO:666 | TCL1 Protein | | |
| SEQ ID NO:667 | Coronin1A DNA | | |
| SEQ ID NO:668 | Coronin1A Protein | | |

Table 8 identifies the putative open reading frames obtained from analyses of the cDNA sequences obtained in SEQ ID NO:1-SEQ ID NO:668 as described above. Shown are the sequence identifiers, the clone name and translation frame, and the start and stop nucleotides in the corresponding DNA sequence used to generate the polypeptide sequence of the open reading frame.

TABLE 8
TRANSLATION OF OPEN READING FRAMES OF IDENTIFIED cDNAs

| Sequence Identifier | | Translation | Beginning and |
|---|---|---|---|
| Number | ORF Identifier | Frame | Ending |
| SEQ ID NO: 669 | ‘41567.1_gaiger.ABI’_1 | frame 1 | from 1 to 79 |
| SEQ ID NO: 670 | ‘41567.1_gaiger.ABI’_2 | frame 3 from 11 to 134 | |
| SEQ ID NO: 671 | ‘41567.1_gaiger.ABI’_3 | frame −1 | from 86 to 135 |
| SEQ ID NO: 672 | ‘41567.1_gaiger.ABI’_4 | frame −3 | from 1 to 108 |
| SEQ ID NO: 673 | ‘41557.1_gaiger.ABI’_1 | frame 1 | from 16 to 73 |
| SEQ ID NO: 674 | ‘41557.1_gaiger.ABI’_2 | frame 2 | from 1 to 109 |
| SEQ ID NO: 675 | ‘41557.1_gaiger.ABI’_3 | frame −1 | from 11 to 110 |
| SEQ ID NO: 676 | ‘41557.1_gaiger.ABI’_4 | frame −3 | from 1 to 103 |
| SEQ ID NO: 677 | ‘41571.1_gaiger.ABI’_1 | frame 3 | from 1 to 89 |
| SEQ ID NO: 678 | ‘41571.1_gaiger.ABI’_2 | frame −1 | from 1 to 89 |
| SEQ ID NO: 679 | ‘41571.1_gaiger.ABI’_3 | frame −2 | from 27 to 85 |
| SEQ ID NO: 680 | ‘41594.1_gaiger.ABI’_1 | frame 3 | from 1 to 123 |
| SEQ ID NO: 681 | ‘41594.1_gaiger.ABI’_2 | frame −2 | from 1 to 85 |
| SEQ ID NO: 682 | ‘41605.1_gaiger.ABI’_1 | frame 3 | from 1 to 85 |
| SEQ ID NO: 683 | ‘41605.1_gaiger.ABI’_2 | frame −3 | from 1 to 123 |
| SEQ ID NO: 684 | ‘41627.1_gaiger.ABI’_1 | frame 1 | from 1 to 161 |
| SEQ ID NO: 685 | ‘41627.1_gaiger.ABI’_2 | frame 2 | from 102 to 161 |
| SEQ ID NO: 686 | ‘41627.1_gaiger.ABI’_3 | frame 3 | from 1 to 67 |
| SEQ ID NO: 687 | ‘41627.1_gaiger.ABI’_4 | frame 3 from 69 to 136 | |
| SEQ ID NO: 688 | ‘41627.1_gaiger.ABI’_5 | frame −2 | from 1 to 106 |
| SEQ ID NO: 689 | ‘41627.1_gaiger.ABI’_6 | frame −3 | from 67 to 160 |
| SEQ ID NO: 690 | ‘41620.1_gaiger.ABI’_1 | frame 1 | from 1 to 151 |
| SEQ ID NO: 691 | ‘41620.1_gaiger.ABI’_2 | frame 3 | from 1 to 59 |
| SEQ ID NO: 692 | ‘41620.1_gaiger.ABI’_3 | frame −1 | from 1 to 85 |
| SEQ ID NO: 693 | ‘41620.1_gaiger.ABI’_4 | frame −1 | from 100 to 152 |
| SEQ ID NO: 694 | ‘41620.1_gaiger.ABI’_5 | frame −2 | from 48 to 109 |
| SEQ ID NO: 695 | ‘41620.1_gaiger.ABI’_6 | frame −3 | from 69 to 119 |
| SEQ ID NO: 696 | ‘41628.1_gaiger.ABI’_1 | frame 1 | from 51 to 121 |
| SEQ ID NO: 697 | ‘41628.1_gaiger.ABI’_2 | frame 2 | from 1 to 97 |
| SEQ ID NO: 698 | ‘41628.1_gaiger.ABI’_3 | frame −3 | from 47 to 98 |
| SEQ ID NO: 699 | ‘41635.1_gaiger.ABI’_1 | frame 1 | from 1 to 70 |
| SEQ ID NO: 700 | ‘41635.1_gaiger.ABI’_2 | frame 2 | from 31 to 127 |
| SEQ ID NO: 701 | ‘41635.1_gaiger.ABI’_3 | frame −1 | from 56 to 127 |
| SEQ ID NO: 702 | ‘41635.1_gaiger.ABI’_4 | frame −2 | from 76 to 126 |
| SEQ ID NO: 703 | ‘41649.1_gaiger.ABI’_1 | frame 1 | from 17 to 77 |
| SEQ ID NO: 704 | ‘41649.1_gaiger.ABI’_2 | frame 3 | from 1 to 56 |
| SEQ ID NO: 705 | ‘41649.1_gaiger.ABI’_3 | frame −2 | from 12 to 87 |
| SEQ ID NO: 706 | ‘41648.1_gaiger.ABI’_1 | frame 3 | from 1 to 154 |
| SEQ ID NO: 707 | ‘41648.1_gaiger.ABI’_2 | frame −1 | from 1 to 67 |
| SEQ ID NO: 708 | ‘41648.1_gaiger.ABI’_3 | frame −2 | from 1 to 116 |
| SEQ ID NO: 709 | ‘41664.1_gaiger.ABI’_1 | frame 3 | from 1 to 125 |
| SEQ ID NO: 710 | ‘41664.1_gaiger.ABI’_2 | frame −2 | from 18 to 87 |
| SEQ ID NO: 711 | ‘41664.1_gaiger.ABI’_3 | frame −3 | from 1 to 53 |
| SEQ ID NO: 712 | ‘41667.1_gaiger.ABI’_1 | frame 1 | from 1 to 56 |
| SEQ ID NO: 713 | ‘41667.1_gaiger.ABI’_2 | frame 2 | from 1 to 56 |
| SEQ ID NO: 714 | ‘41667.1_gaiger.ABI’_3 | frame −2 | from 1 to 56 |
| SEQ ID NO: 715 | ‘41687.1_gaiger.ABI’_1 | frame 1 | from 35 to 154 |
| SEQ ID NO: 716 | ‘41687.1_gaiger.ABI’_2 | frame 2 | from 102 to 153 |
| SEQ ID NO: 717 | ‘41687.1_gaiger.ABI’_3 | frame −1 | from 50 to 109 |
| SEQ ID NO: 718 | ‘41687.1_gaiger.ABI’_4 | frame −3 | from 102 to 153 |
| SEQ ID NO: 719 | ‘41708.1_gaiger.ABI’_1 | frame 1 | from 1 to 53 |
| SEQ ID NO: 720 | ‘41708.1_gaiger.ABI’_2 | frame 2 | from 1 to 59 |
| SEQ ID NO: 721 | ‘41708.1_gaiger.ABI’_3 | frame 3 | from 1 to 68 |
| SEQ ID NO: 722 | ‘41708.1_gaiger.ABI’_4 | frame −1 | from 1 to 51 |
| SEQ ID NO: 723 | ‘41708.1_gaiger.ABI’_5 | frame −2 | from 17 to 68 |
| SEQ ID NO: 724 | ‘41721.1_gaiger.ABI’_1 | frame −2 | from 1 to 57 |
| SEQ ID NO: 725 | ‘41721.1_gaiger.ABI’_2 | frame −3 | from 1 to 97 |
| SEQ ID NO: 726 | ‘41746.1_gaiger.ABI’_1 | frame 1 | from 1 to 65 |
| SEQ ID NO: 727 | ‘41746.1_gaiger.ABI’_2 | frame 2 | from 1 to 60 |
| SEQ ID NO: 728 | ‘41746.1_gaiger.ABI’_3 | frame −2 | from 7 to 65 |
| SEQ ID NO: 729 | ‘41751.1_gaiger.ABI’_1 | frame 1 | from 27 to 82 |
| SEQ ID NO: 730 | ‘41751.1_gaiger.ABI’_2 | frame 3 | from 1 to 50 |
| SEQ ID NO: 731 | ‘41751.1_gaiger.ABI’_3 | frame −2 | from 1 to 70 |
| SEQ ID NO: 732 | ‘41751.1_gaiger.ABI’_4 | frame −3 | from 1 to 53 |
| SEQ ID NO: 733 | ‘41762.1_gaiger.ABI’_1 | frame 1 | from 1 to 76 |
| SEQ ID NO: 734 | ‘41762.1_gaiger.ABI’_2 | frame 2 | from 1 to 96 |
| SEQ ID NO: 735 | ‘41793.1_gaiger.ABI’_1 | frame 3 | from 1 to 85 |
| SEQ ID NO: 736 | ‘41793.1_gaiger.ABI’_2 | frame −3 | from 1 to 87 |
| SEQ ID NO: 737 | ‘41794.1_gaiger.ABI’_1 | frame 1 | from 1 to 125 |
| SEQ ID NO: 738 | ‘41794.1_gaiger.ABI’_2 | frame −3 | from 1 to 85 |
| SEQ ID NO: 739 | ‘41807.1_gaiger.ABI’_1 | frame 1 | from 1 to 67 |
| SEQ ID NO: 740 | ‘41807.1_gaiger.ABI’_2 | frame 2 | from 11 to 107 |
| SEQ ID NO: 741 | ‘41807.1_gaiger.ABI’_3 | frame −1 | from 51 to 107 |
| SEQ ID NO: 742 | ‘41802.1_gaiger.ABI’_1 | frame 3 | from 1 to 143 |
| SEQ ID NO: 743 | ‘41802.1_gaiger.ABI’_2 | frame −2 | from 4 to 56 |
| SEQ ID NO: 744 | ‘41802.1_gaiger.ABI’_3 | frame −3 | from 1 to 105 |
| SEQ ID NO: 745 | ‘41804.1_gaiger.ABI’_1 | frame 1 | from 1 to 59 |
| SEQ ID NO: 746 | ‘41804.1_gaiger.ABI’_2 | frame 2 | from 15 to 92 |
| SEQ ID NO: 747 | ‘41804.1_gaiger.ABI’_3 | frame 3 | from 33 to 82 |
| SEQ ID NO: 748 | ‘41804.1_gaiger.ABI’_4 | frame 3 | from 84 to 139 |
| SEQ ID NO: 749 | ‘41804.1_gaiger.ABI’_5 | frame −2 | from 22 to 139 |
| SEQ ID NO: 750 | ‘41804.1_gaiger.ABI’_6 | frame −3 | from 1 to 60 |
| SEQ ID NO: 751 | ‘41810.1_gaiger.ABI’_1 | frame 1 | from 1 to 67 |
| SEQ ID NO: 752 | ‘41810.1_gaiger.ABI’_2 | frame −1 | from 1 to 67 |
| SEQ ID NO: 753 | ‘41847.1_gaiger.ABI’_1 | frame −1 | from 1 to 97 |
| SEQ ID NO: 754 | ‘41847.1_gaiger.ABI’_2 | frame −3 | from 1 to 56 |
| SEQ ID NO: 755 | ‘41865.1_gaiger.ABI’_1 | frame 1 | from 1 to 139 |
| SEQ ID NO: 756 | ‘41865.1_gaiger.ABI’_2 | frame 3 | from 58 to 108 |
| SEQ ID NO: 757 | ‘41865.1_gaiger.ABI’_3 | frame −2 | from 1 to 92 |
| SEQ ID NO: 758 | ‘41859.1_gaiger.ABI’_1 | frame 1 | from 86 to 138 |
| SEQ ID NO: 759 | ‘41859.1_gaiger.ABI’_2 | frame 3 | from 1 to 108 |
| SEQ ID NO: 760 | ‘41859.1_gaiger.ABI’_3 | frame −1 | from 18 to 95 |
| SEQ ID NO: 761 | ‘41859.1_gaiger.ABI’_4 | frame −3 | from 27 to 150 |
| SEQ ID NO: 762 | ‘41878.1_gaiger.ABI’_1 | frame 2 | from 70 to 131 |
| SEQ ID NO: 763 | ‘41878.1_gaiger.ABI’_2 | frame −3 | from 30 to 88 |
| SEQ ID NO: 764 | ‘41869.1_gaiger.ABI’_1 | frame 1 | from 41 to 127 |
| SEQ ID NO: 765 | ‘41869.1_gaiger.ABI’_2 | frame 3 | from 1 to 55 |
| SEQ ID NO: 766 | ‘41869.1_gaiger.ABI’_3 | frame −3 | from 1 to 121 |
| SEQ ID NO: 767 | ‘41888.1_gaiger.ABI’_1 | frame 3 | from 22 to 81 |
| SEQ ID NO: 768 | ‘41907.1_gaiger.ABI’_1 | frame 1 | from 1 to 73 |
| SEQ ID NO: 769 | ‘41907.1_gaiger.ABI’_2 | frame 2 | from 29 to 102 |
| SEQ ID NO: 770 | ‘41907.1_gaiger.ABI’_3 | frame 3 | from 47 to 96 |
| SEQ ID NO: 771 | ‘41907.1_gaiger.ABI’_4 | frame −1 | from 42 to 103 |
| SEQ ID NO: 772 | ‘41907.1_gaiger.ABI’_5 | frame −2 | from 44 to 102 |
| SEQ ID NO: 773 | ‘41907.1_gaiger.ABI’_6 | frame −3 | from 1 to 102 |
| SEQ ID NO: 774 | ‘41908.1_gaiger.ABI’_1 | frame 1 | from 1 to 102 |
| SEQ ID NO: 775 | ‘41908.1_gaiger.ABI’_2 | frame 3 | from 67 to 120 |
| SEQ ID NO: 776 | ‘41908.1_gaiger.ABI’_3 | frame −1 | from 54 to 121 |
| SEQ ID NO: 777 | ‘41908.1_gaiger.ABI’_4 | frame −2 | from 1 to 50 |
| SEQ ID NO: 778 | ‘41912.1_gaiger.ABI’_1 | frame 2 | from 1 to 138 |
| SEQ ID NO: 779 | ‘41912.1_gaiger.ABI’_2 | frame −2 | from 34 to 93 |
| SEQ ID NO: 780 | ‘41912.1_gaiger.ABI’_3 | frame −3 | from 60 to 125 |
| SEQ ID NO: 781 | ‘41916.1_gaiger.ABI’_1 | frame 2 | from 1 to 84 |
| SEQ ID NO: 782 | ‘41916.1_gaiger.ABI’_2 | frame −1 | from 1 to 84 |
| SEQ ID NO: 783 | ‘41925.1_gaiger.ABI’_1 | frame 1 | from 9 to 59 |
| SEQ ID NO: 784 | ‘41925.1_gaiger.ABI’_2 | frame 2 | from 1 to 59 |
| SEQ ID NO: 785 | ‘41925.1_gaiger.ABI’_3 | frame −2 | from 1 to 59 |
| SEQ ID NO: 786 | ‘41925.1_gaiger.ABI’_4 | frame −3 | from 1 to 58 |
| SEQ ID NO: 787 | ‘41929.1_gaiger.ABI’_1 | frame 1 | from 1 to 52 |
| SEQ ID NO: 788 | ‘41930.1_gaiger.ABI’_1 | frame −1 | from 1 to 55 |
| SEQ ID NO: 789 | ‘41930.1_gaiger.ABI’_2 | frame −2 | from 1 to 95 |
| SEQ ID NO: 790 | ‘41933.1_gaiger.ABI’_1 | frame 1 | from 1 to 90 |
| SEQ ID NO: 791 | ‘41933.1_gaiger.ABI’_2 | frame 2 from 36 to 90 | |
| SEQ ID NO: 792 | ‘41944.1_gaiger.ABI’_1 | frame 1 | from 1 to 56 |
| SEQ ID NO: 793 | ‘41944.1_gaiger.ABI’_2 | frame 2 from 1 to 177 | |
| SEQ ID NO: 794 | ‘41944.1_gaiger.ABI’_3 | frame 3 | from 37 to 92 |
| SEQ ID NO: 795 | ‘41944.1_gaiger.ABI’_4 | frame −1 | from 47 to 116 |
| SEQ ID NO: 796 | ‘41944.1_gaiger.ABI’_5 | frame −1 | from 125 to 177 |
| SEQ ID NO: 797 | ‘41944.1_gaiger.ABI’_6 | frame −2 | from 32 to 177 |
| SEQ ID NO: 798 | ‘41944.1_gaiger.ABI’_7 | frame −3 | from 120 to 177 |
| SEQ ID NO: 799 | ‘41986.1_gaiger.ABI’_1 | frame 3 | from 1 to 110 |
| SEQ ID NO: 800 | ‘41986.1_gaiger.ABI’_2 | frame −1 | from 1 to 110 |
| SEQ ID NO: 801 | ‘41986.1_gaiger.ABI’_3 | frame −3 | from 22 to 91 |
| SEQ ID NO: 802 | ‘42017.1_gaiger.ABI’_1 | frame 2 | from 78 to 130 |
| SEQ ID NO: 803 | ‘42017.1_gaiger.ABI’_2 | frame 3 | from 1 to 85 |
| SEQ ID NO: 804 | ‘42017.1_gaiger.ABI’_3 | frame −3 | from 1 to 129 |
| SEQ ID NO: 805 | ‘42033.1_gaiger.ABI’_1 | frame 3 | from 1 to 140 |
| SEQ ID NO: 806 | ‘42033.1_gaiger.ABI’_2 | frame −2 | from 1 to 71 |
| SEQ ID NO: 807 | ‘42033.1_gaiger.ABI’_3 | frame −3 | from 1 to 120 |
| SEQ ID NO: 808 | ‘42040.1_gaiger.ABI’_1 | frame 3 | from 1 to 80 |
| SEQ ID NO: 809 | ‘42041.1_gaiger.ABI’_1 | frame −3 | from 1 to 63 |
| SEQ ID NO: 810 | ‘42053.1_gaiger.ABI’_1 | frame 3 | from 1 to 123 |
| SEQ ID NO: 811 | ‘42053.1_gaiger.ABI’_2 | frame −1 | from 17 to 66 |
| SEQ ID NO: 812 | ‘42053.1_gaiger.ABI’_3 | frame −3 | from 1 to 85 |
| SEQ ID NO: 813 | ‘42101.1_gaiger.ABI’_1 | frame 3 | from 53 to 123 |
| SEQ ID NO: 814 | ‘42101.1_gaiger.ABI’_2 | frame −2 | from 1 to 124 |
| SEQ ID NO: 815 | ‘42131.1_gaiger.ABI’_1 | frame 3 | from 1 to 114 |
| SEQ ID NO: 816 | ‘42131.1_gaiger.ABI’_2 | frame −1 | from 8 to 77 |
| SEQ ID NO: 817 | R0232:A08_1 | frame −2 | from 4 to 64 |
| SEQ ID NO: 818 | R0232:C10_1 | frame 3 | from 1 to 65 |
| SEQ ID NO: 819 | R0232:C10_2 | frame −2 | from 1 to 61 |
| SEQ ID NO: 820 | R0233:A12_1 | frame 3 | from 1 to 141 |
| SEQ ID NO: 821 | R0233:A12_2 | frame −3 | from 24 to 124 |
| SEQ ID NO: 822 | R0233:A06_1 | frame 1 | from 12 to 77 |
| SEQ ID NO: 823 | R0233:A06_2 | frame 3 | from 2 to 76 |
| SEQ ID NO: 824 | R0233:A06_3 | frame −3 | from 1 to 59 |
| SEQ ID NO: 825 | R0233:A08_1 | frame 1 | from 1 to 59 |
| SEQ ID NO: 826 | R0233:A08_2 | frame −1 | from 1 to 63 |
| SEQ ID NO: 827 | R0233:B10_1 | frame 3 | from 1 to 85 |
| SEQ ID NO: 828 | R0233:B10_2 | frame −3 | from 1 to 85 |
| SEQ ID NO: 829 | R0233:B04_1 | frame 2 | from 76 to 136 |
| SEQ ID NO: 830 | R0233:B04_2 | frame −3 | from 1 to 103 |
| SEQ ID NO: 831 | R0233:C04_1 | frame 3 | from 1 to 83 |
| SEQ ID NO: 832 | R0233:C04_2 | frame −3 | from 1 to 119 |
| SEQ ID NO: 833 | R0233:D01_1 | frame 3 | from 1 to 85 |
| SEQ ID NO: 834 | R0233:D01_2 | frame −1 | from 2 to 122 |
| SEQ ID NO: 835 | R0233:D02_1 | frame 3 | from 1 to 127 |
| SEQ ID NO: 836 | R0233:D02_2 | frame −1 | from 1 to 127 |
| SEQ ID NO: 837 | R0233:F10_1 | frame 3 | from 1 to 85 |
| SEQ ID NO: 838 | R0233:F10_2 | frame −3 | from 1 to 123 |
| SEQ ID NO: 839 | R0233:F05_1 | frame 3 | from 1 to 85 |
| SEQ ID NO: 840 | R0233:F05_2 | frame −2 | from 58 to 111 |
| SEQ ID NO: 841 | R0233:F05_3 | frame −3 | from 1 to 110 |
| SEQ ID NO: 842 | R0233:F07_1 | frame 3 | from 1 to 85 |
| SEQ ID NO: 843 | R0233:F07_2 | frame −1 | from 1 to 125 |
| SEQ ID NO: 844 | ‘42324.1_gaiger.ABI’_1 | frame 1 | from 1 to 94 |
| SEQ ID NO: 845 | ‘42324.1_gaiger.ABI’_2 | frame 2 | from 1 to 57 |
| SEQ ID NO: 846 | ‘42324.1_gaiger.ABI’_3 | frame 3 | from 38 to 130 |
| SEQ ID NO: 847 | ‘42324.1_gaiger.ABI’_4 | frame −1 | from 10 to 130 |
| SEQ ID NO: 848 | ‘42324.1_gaiger.ABI’_5 | frame −2 | from 1 to 54 |
| SEQ ID NO: 849 | ‘42324.1_gaiger.ABI’_6 | frame −2 | from 72 to 130 |
| SEQ ID NO: 850 | ‘42324.1_gaiger.ABI’_7 | frame −3 | from 1 to 67 |
| SEQ ID NO: 851 | ‘42324.1_gaiger.ABI’_8 | frame −3 | from 76 to 130 |
| SEQ ID NO: 852 | ‘42349.1_gaiger.ABI’_1 | frame 3 | from 1 to 146 |
| SEQ ID NO: 853 | ‘42349.1_gaiger.ABI’_2 | frame −2 | from 1 to 137 |
| SEQ ID NO: 854 | ‘42379.1_gaiger.ABI’_1 | frame 3 | from 1 to 59 |
| SEQ ID NO: 855 | ‘42379.1_gaiger.ABI’_2 | frame −2 | from 1 to 59 |
| SEQ ID NO: 856 | ‘42396.1_gaiger.ABI’_1 | frame −1 | from 1 to 50 |
| SEQ ID NO: 857 | ‘42396.1_gaiger.ABI’_2 | frame −2 | from 22 to 82 |
| SEQ ID NO: 858 | ‘42424.1_gaiger.ABI’_1 | frame 3 | from 1 to 85 |
| SEQ ID NO: 859 | ‘42424.1_gaiger.ABI’_2 | frame −3 | from 1 to 123 |
| SEQ ID NO: 860 | ‘42438.1_gaiger.ABI’_1 | frame 1 | from 1 to 123 |
| SEQ ID NO: 861 | ‘42438.1_gaiger.ABI’_2 | frame −3 | from 53 to 123 |
| SEQ ID NO: 862 | ‘42447.1_gaiger.ABI’_1 | frame 1 | from 1 to 57 |
| SEQ ID NO: 863 | ‘42447.1_gaiger.ABI’_2 | frame 2 | from 33 to 97 |
| SEQ ID NO: 864 | ‘42447.1_gaiger.ABI’_3 | frame 3 | from 1 to 72 |
| SEQ ID NO: 865 | ‘42447.1_gaiger.ABI’_4 | frame −2 | from 26 to 97 |
| SEQ ID NO: 866 | ‘42524.1;gaiger.ABI’_1 | frame 2 | from 1 to 69 |
| SEQ ID NO: 867 | ‘42524.1;gaiger.ABI’_2 | frame 3 | from 1 to 59 |
| SEQ ID NO: 868 | ‘42555.1;gaiger.ABI’_1 | frame 3 | from 1 to 115 |
| SEQ ID NO: 869 | ‘42555.1;gaiger.ABI’_2 | frame −2 | from 35 to 131 |
| SEQ ID NO: 870 | ‘42555.1;gaiger.ABI’_3 | frame −3 | from 1 to 75 |
| SEQ ID NO: 871 | ‘42560.1;gaiger.ABI’_1 | frame 1 | from 1 to 67 |
| SEQ ID NO: 872 | ‘42560.1;gaiger.ABI’_2 | frame −3 | from 1 to 66 |
| SEQ ID NO: 873 | ‘42594.1_gaiger.ABI’_1 | frame 2 | from 56 to 118 |
| SEQ ID NO: 874 | ‘42594.1_gaiger.ABI’_2 | frame −1 | from 42 to 118 |
| SEQ ID NO: 875 | ‘42602.1_gaiger.ABI’_1 | frame 1 | from 1 to 97 |
| SEQ ID NO: 876 | ‘42602.1_gaiger.ABI’_2 | frame 3 | from 1 to 76 |
| SEQ ID NO: 877 | ‘42665.1_gaiger.ABI’_1 | frame 1 | from 1 to 94 |
| SEQ ID NO: 878 | ‘42665.1_gaiger.ABI’_2 | frame 3 | from 35 to 94 |
| SEQ ID NO: 879 | ‘42665.1_gaiger.ABI’_3 | frame −1 | from 35 to 94 |
| SEQ ID NO: 880 | ‘42665.1_gaiger.ABI’_4 | frame −3 | from 12 to 73 |
| SEQ ID NO: 881 | ‘42703.1_gaiger.ABI’_1 | frame 2 | from 25 to 95 |
| SEQ ID NO: 882 | ‘42703.1_gaiger.ABI’_2 | frame −2 | from 10 to 82 |
| SEQ ID NO: 883 | ‘42709.1_gaiger.ABI’_1 | frame 2 | from 1 to 118 |
| SEQ ID NO: 884 | ‘42709.1_gaiger.ABI’_2 | frame −3 | from 53 to 118 |
| SEQ ID NO: 885 | ‘42756.1_gaiger.ABI’_1 | frame 3 | from 1 to 109 |
| SEQ ID NO: 886 | ‘42756.1_gaiger.ABI’_2 | frame −2 | from 1 to 85 |
| SEQ ID NO: 887 | ‘42756.1_gaiger.ABI’_3 | frame −3 | from 1 to 51 |
| SEQ ID NO: 888 | R0234:A06_1 | frame 3 | from 1 to 118 |
| SEQ ID NO: 889 | R0234:A06_2 | frame −2 | from 1 to 80 |
| SEQ ID NO: 890 | R0234:A07_1 | frame 1 | from 1 to 62 |
| SEQ ID NO: 891 | R0234:A07_2 | frame 2 | from 6 to 102 |
| SEQ ID NO: 892 | R0234:A07_3 | frame −1 | from 51 to 102 |
| SEQ ID NO: 893 | R0234:B03_1 | frame 3 | from 1 to 68 |
| SEQ ID NO: 894 | R0234:B03_2 | frame −3 | from 2 to 63 |
| SEQ ID NO: 895 | R0234:B06_1 | frame 3 | from 1 to 85 |
| SEQ ID NO: 896 | R0234:B06_2 | frame −3 | from 1 to 123 |
| SEQ ID NO: 897 | R0234:B09_1 | frame 1 | from 1 to 115 |
| SEQ ID NO: 898 | R0234:B09_2 | frame −3 | from 53 to 115 |
| SEQ ID NO: 899 | R0234:C02_1 | frame 3 | from 1 to 85 |
| SEQ ID NO: 900 | R0234:C02_2 | frame 3 | from 87 to 139 |
| SEQ ID NO: 901 | R0234:C02_3 | frame −3 | from 1 to 139 |
| SEQ ID NO: 902 | R0234:C06_1 | frame 3 | from 1 to 85 |
| SEQ ID NO: 903 | R0234:C06_2 | frame −2 | from 1 to 107 |
| SEQ ID NO: 904 | R0234:D08_1 | frame 3 | from 1 to 55 |
| SEQ ID NO: 905 | R0234:D08_2 | frame −1 | from 1 to 55 |
| SEQ ID NO: 906 | R0234:E01_1 | frame 3 | from 1 to 101 |
| SEQ ID NO: 907 | R0234:E01_2 | frame −3 | from 1 to 101 |
| SEQ ID NO: 908 | R0234:E12_1 | frame 2 | from 78 to 134 |
| SEQ ID NO: 909 | R0234:E12_2 | frame 3 | from 1 to 189 |
| SEQ ID NO: 910 | R0234:E12_3 | frame −2 | from 8 to 120 |
| SEQ ID NO: 911 | R0234:E12_4 | frame −3 | from 28 to 77 |
| SEQ ID NO: 912 | R0234:E12_5 | frame −3 | from 105 to 189 |
| SEQ ID NO: 913 | R0234:E02_1 | frame 3 | from 1 to 85 |
| SEQ ID NO: 914 | R0234:E02_2 | frame −3 | from 1 to 111 |
| SEQ ID NO: 915 | R0234:E04_1 | frame 1 | from 40 to 114 |
| SEQ ID NO: 916 | R0234:E04_2 | frame 3 | from 1 to 54 |
| SEQ ID NO: 917 | R0234:E04_3 | frame −1 | from 1 to 109 |
| SEQ ID NO: 918 | R0234:E05_1 | frame 3 | from 1 to 85 |
| SEQ ID NO: 919 | R0234:E05_2 | frame −1 | from 1 to 52 |
| SEQ ID NO: 920 | R0234:E05_3 | frame −2 | from 1 to 121 |
| SEQ ID NO: 921 | R0234:F02_1 | frame 3 | from 1 to 109 |
| SEQ ID NO: 922 | R0234:F02_2 | frame −2 | from 1 to 109 |
| SEQ ID NO: 923 | R0234:F04_1 | frame 3 | from 1 to 83 |
| SEQ ID NO: 924 | R0234:F04_2 | frame −2 | from 1 to 122 |
| SEQ ID NO: 925 | R0234:G01_1 | frame 3 | from 1 to 84 |
| SEQ ID NO: 926 | R0234:G11_1 | frame 3 | from 1 to 121 |
| SEQ ID NO: 927 | R0234:G11_2 | frame −2 | from 51 to 121 |
| SEQ ID NO: 928 | R0234:G12_1 | frame 2 | from 1 to 150 |
| SEQ ID NO: 929 | R0234:G12_2 | frame −2 | from 61 to 113 |
| SEQ ID NO: 930 | R0234:G12_3 | frame −3 | from 24 to 124 |
| SEQ ID NO: 931 | R0234:G02_1 | frame 3 | from 1 to 123 |
| SEQ ID NO: 932 | R0234:G02_2 | frame −3 | from 1 to 85 |
| SEQ ID NO: 933 | R0234:G04_1 | frame 2 | from 1 to 150 |
| SEQ ID NO: 934 | R0234:G04_2 | frame −3 | from 24 to 124 |
| SEQ ID NO: 935 | R0234:G09_1 | frame 1 | from 1 to 61 |
| SEQ ID NO: 936 | R0234:G09_2 | frame 1 | from 74 to 187 |
| SEQ ID NO: 937 | R0234:G09_3 | frame 2 | from 123 to 186 |
| SEQ ID NO: 938 | R0234:G09_4 | frame 3 | from 1 to 82 |
| SEQ ID NO: 939 | R0234:G09_5 | frame 3 | from 84 to 171 |
| SEQ ID NO: 940 | R0234:G09_6 | frame −2 | from 90 to 155 |
| SEQ ID NO: 941 | R0234:G09_7 | frame −3 | from 29 to 164 |
| SEQ ID NO: 942 | R0234:H01_1 | frame 3 | from 1 to 84 |
| SEQ ID NO: 943 | R0234:H06_1 | frame 3 | from 1 to 85 |
| SEQ ID NO: 944 | R0234:H06_2 | frame −1 | from 1 to 121 |
| SEQ ID NO: 945 | R0235:B01_1 | frame 3 | from 1 to 119 |
| SEQ ID NO: 946 | R0235:B01_2 | frame −1 | from 8 to 128 |
| SEQ ID NO: 947 | R0235:B01_3 | frame −3 | from 3 to 58 |
| SEQ ID NO: 948 | R0235:B11_1 | frame 3 | from 1 to 62 |
| SEQ ID NO: 949 | R0235:B04_1 | frame 3 | from 1 to 101 |
| SEQ ID NO: 950 | R0235:B04_2 | frame −1 | from 1 to 102 |
| SEQ ID NO: 951 | R0235:B05_1 | frame 3 | from 1 to 67 |
| SEQ ID NO: 952 | R0235:B05_2 | frame −1 | from 1 to 67 |
| SEQ ID NO: 953 | R0235:B07_1 | frame 3 | from 1 to 83 |
| SEQ ID NO: 954 | R0235:B09_1 | frame 1 | from 1 to 58 |
| SEQ ID NO: 955 | R0235:B09_2 | frame 2 | from 2 to 78 |
| SEQ ID NO: 956 | R0235:B09_3 | frame 3 | from 34 to 88 |
| SEQ ID NO: 957 | R0235:C07_1 | frame 3 | from 1 to 69 |
| SEQ ID NO: 958 | R0235:C07_2 | frame −2 | from 1 to 69 |
| SEQ ID NO: 959 | R0235:C09_1 | frame −1 | from 1 to 97 |
| SEQ ID NO: 960 | R0235:C09_2 | frame −3 | from 1 to 56 |
| SEQ ID NO: 961 | R0235:D11_1 | frame 1 | from 1 to 87 |
| SEQ ID NO: 962 | R0235:D11_2 | frame 2 | from 74 to 136 |
| SEQ ID NO: 963 | R0235:D11_3 | frame 3 | from 1 to 76 |
| SEQ ID NO: 964 | R0235:D11_4 | frame −1 | from 15 to 85 |
| SEQ ID NO: 965 | R0235:D11_5 | frame −2 | from 6 to 94 |
| SEQ ID NO: 966 | R0235:E10_1 | frame 3 | from 1 to 66 |
| SEQ ID NO: 967 | R0235:E12_1 | frame 3 | from 1 to 51 |
| SEQ ID NO: 968 | R0235:E12_2 | frame −1 | from 1 to 51 |
| SEQ ID NO: 969 | R0235:E02_1 | frame 3 | from 1 to 52 |
| SEQ ID NO: 970 | R0235:F01_1 | frame 3 | from 1 to 66 |
| SEQ ID NO: 971 | R0235:F02_1 | frame 3 | from 1 to 56 |
| SEQ ID NO: 972 | R0235:F02_2 | frame −2 | from 11 to 65 |
| SEQ ID NO: 973 | R0235:F06_1 | frame 3 | from 24 to 124 |
| SEQ ID NO: 974 | R0235:F06_2 | frame −2 | from 1 to 150 |
| SEQ ID NO: 975 | R0235:F09_1 | frame 3 | from 1 to 53 |
| SEQ ID NO: 976 | R0235:F09_2 | frame −1 | from 1 to 53 |
| SEQ ID NO: 977 | R0235:G07_1 | frame 3 | from 1 to 97 |
| SEQ ID NO: 978 | R0235:G07_2 | frame −2 | from 1 to 59 |
| SEQ ID NO: 979 | R0235:H06_1 | frame 3 | from 1 to 83 |
| SEQ ID NO: 980 | R0235:H06_2 | frame −3 | from 1 to 60 |
| SEQ ID NO: 981 | R0235:H08_1 | frame 3 | from 1 to 123 |
| SEQ ID NO: 982 | R0235:H08_2 | frame −3 | from 1 to 123 |
| SEQ ID NO: 983 | R0236:A06_1 | frame 2 | from 1 to 150 |
| SEQ ID NO: 984 | R0236:A06_2 | frame −2 | from 25 to 125 |
| SEQ ID NO: 985 | R0236:A09_1 | frame 3 | from 1 to 122 |
| SEQ ID NO: 986 | R0236:A09_2 | frame −1 | from 54 to 122 |
| SEQ ID NO: 987 | R0236:C01_1 | frame 3 | from 1 to 118 |
| SEQ ID NO: 988 | R0236:C01_2 | frame −2 | from 1 to 80 |
| SEQ ID NO: 989 | R0236:F12_1 | frame 1 | from 17 to 79 |
| SEQ ID NO: 990 | R0236:F12_2 | frame 3 | from 1 to 56 |
| SEQ ID NO: 991 | R0236:F05_1 | frame 3 | from 1 to 123 |
| SEQ ID NO: 992 | R0236:F05_2 | frame −3 | from 1 to 85 |
| SEQ ID NO: 993 | R0236:F06_1 | frame 3 | from 1 to 123 |
| SEQ ID NO: 994 | R0236:F06_2 | frame −3 | from 1 to 85 |
| SEQ ID NO: 995 | R0236:G08_1 | frame 2 | from 1 to 88 |
| SEQ ID NO: 996 | R0236:G08_2 | frame 3 | from 34 to 88 |
| SEQ ID NO: 997 | R0249:A11_1 | frame 3 | from 1 to 83 |
| SEQ ID NO: 998 | R0249:A11_2 | frame −3 | from 1 to 121 |
| SEQ ID NO: 999 | R0249:B04_1 | frame −2 | from 1 to 56 |
| SEQ ID NO: 1000 | R0249:B04_2 | frame −3 | from 1 to 96 |
| SEQ ID NO: 1001 | R0249:B06_1 | frame −1 | from 1 to 81 |
| SEQ ID NO: 1002 | R0249:D11_1 | frame 2 | from 1 to 170 |
| SEQ ID NO: 1003 | R0249:D11_2 | frame 3 | from 41 to 101 |
| SEQ ID NO: 1004 | R0249:D11_3 | frame 3 | from 103 to 153 |
| SEQ ID NO: 1005 | R0249:D11_4 | frame −1 | from 1 to 59 |
| SEQ ID NO: 1006 | R0249:D11_5 | frame −1 | from 79 to 139 |
| SEQ ID NO: 1007 | R0249:D11_6 | frame −3 | from 65 to 170 |
| SEQ ID NO: 1008 | R0249:E11_1 | frame 3 | from 1 to 59 |
| SEQ ID NO: 1009 | R0249:E11_2 | frame −3 | from 1 to 59 |
| SEQ ID NO: 1010 | R0249:E06_1 | frame 3 | from 1 to 85 |
| SEQ ID NO: 1011 | R0249:E06_2 | frame −3 | from 1 to 123 |
| SEQ ID NO: 1012 | R0249:H09_1 | frame 3 | from 1 to 83 |
| SEQ ID NO: 1013 | R0249:H09_2 | frame −2 | from 1 to 83 |
| SEQ ID NO: 1014 | R0250:C09_1 | frame 1 | from 1 to 55 |
| SEQ ID NO: 1015 | R0250:C09_2 | frame 1 | from 117 to 166 |
| SEQ ID NO: 1016 | R0250:C09_3 | frame 3 | from 30 to 88 |
| SEQ ID NO: 1017 | R0250:C09_4 | frame 3 | from 90 to 165 |
| SEQ ID NO: 1018 | R0250:C09_5 | frame −1 | from 74 to 125 |
| SEQ ID NO: 1019 | R0250:C09_6 | frame −3 | from 1 to 165 |
| SEQ ID NO: 1020 | R0250:D10_1 | frame 3 | from 1 to 85 |
| SEQ ID NO: 1021 | R0250:D10_2 | frame −3 | from 1 to 123 |
| SEQ ID NO: 1022 | R0250:D03_1 | frame 1 | from 17 to 66 |
| SEQ ID NO: 1023 | R0250:D03_2 | frame 3 | from 1 to 80 |
| SEQ ID NO: 1024 | R0250:D03_3 | frame −2 | from 1 to 80 |
| SEQ ID NO: 1025 | R0250:E09_1 | frame 3 | from 1 to 101 |
| SEQ ID NO: 1026 | R0250:E09_2 | frame −3 | from 1 to 63 |
| SEQ ID NO: 1027 | R0250:F09_1 | frame 2 | from 62 to 136 |
| SEQ ID NO: 1028 | R0250:F09_2 | frame 3 | from 69 to 145 |
| SEQ ID NO: 1029 | R0250:F09_3 | frame −1 | from 1 to 82 |
| SEQ ID NO: 1030 | R0250:F09_4 | frame −1 | from 84 to 167 |
| SEQ ID NO: 1031 | R0250:F09_5 | frame −2 | from 1 to 60 |
| SEQ ID NO: 1032 | R0250:G01_1 | frame 1 | from 17 to 87 |
| SEQ ID NO: 1033 | R0250:G01_2 | frame 2 | from 1 to 77 |
| SEQ ID NO: 1034 | R0250:G01_3 | frame 2 | from 126 to 179 |
| SEQ ID NO: 1035 | R0250:G01_4 | frame −1 | from 111 to 160 |
| SEQ ID NO: 1036 | R0250:G01_5 | frame −2 | from 33 to 101 |
| SEQ ID NO: 1037 | R0250:G01_6 | frame −3 | from 1 to 61 |
| SEQ ID NO: 1038 | R0250:G01_7 | frame −3 | from 63 to 121 |
| SEQ ID NO: 1039 | R0250:G01_8 | frame −3 | from 123 to 178 |
| SEQ ID NO: 1040 | R0251:A12_1 | frame 3 | from 1 to 85 |
| SEQ ID NO: 1041 | R0251:A12_2 | frame −3 | from 1 to 123 |
| SEQ ID NO: 1042 | R0251:A05_1 | frame 3 | from 1 to 96 |
| SEQ ID NO: 1043 | R0251:A05_2 | frame −1 | from 1 to 96 |
| SEQ ID NO: 1044 | R0251:B09_1 | frame 3 | from 1 to 85 |
| SEQ ID NO: 1045 | R0251:B09_2 | frame −3 | from 1 to 90 |
| SEQ ID NO: 1046 | R0251:D01_1 | frame 2 | from 1 to 124 |
| SEQ ID NO: 1047 | R0251:D01_2 | frame −3 | from 53 to 123 |
| SEQ ID NO: 1048 | R0251:E03_1 | frame 3 | from 1 to 95 |
| SEQ ID NO: 1049 | R0251:E03_2 | frame −2 | from 1 to 57 |
| SEQ ID NO: 1050 | R0251:E06_1 | frame 3 | from 1 to 98 |
| SEQ ID NO: 1051 | R0251:E06_2 | frame −2 | from 1 to 60 |
| SEQ ID NO: 1052 | R0251:F12_1 | frame 1 | from 51 to 110 |
| SEQ ID NO: 1053 | R0251:F12_2 | frame −1 | from 32 to 111 |
| SEQ ID NO: 1054 | R0251:F12_3 | frame −2 | from 35 to 131 |
| SEQ ID NO: 1055 | R0251:G06_1 | frame −1 | from 1 to 97 |
| SEQ ID NO: 1056 | R0251:G06_2 | frame −3 | from 1 to 56 |
| SEQ ID NO: 1057 | R0252:A08_1 | frame 1 | from 1 to 64 |
| SEQ ID NO: 1058 | R0252:A08_2 | frame 2 | from 12 to 64 |
| SEQ ID NO: 1059 | R0252:A08_3 | frame −1 | from 1 to 51 |
| SEQ ID NO: 1060 | R0252:A08_4 | frame −2 | from 1 to 64 |
| SEQ ID NO: 1061 | R0252:D02_1 | frame 3 | from 1 to 85 |
| SEQ ID NO: 1062 | R0252:D02_2 | frame −3 | from 1 to 123 |
| SEQ ID NO: 1063 | R0252:E04_1 | frame 1 | from 1 to 59 |
| SEQ ID NO: 1064 | R0252:E04_2 | frame 2 from 57 to 107 | |
| SEQ ID NO: 1065 | R0252:E04_3 | frame 3 from 35 to 154 | |
| SEQ ID NO: 1066 | R0252:E04_4 | frame −1 | from 22 to 110 |
| SEQ ID NO: 1067 | R0252:E04_5 | frame −3 | from 1 to 60 |
| SEQ ID NO: 1068 | R0252:E04_6 | frame −3 | from 91 to 154 |
| SEQ ID NO: 1069 | R0252:E06_1 | frame 1 | from 1 to 59 |
| SEQ ID NO: 1070 | R0252:E06_2 | frame 2 | from 57 to 107 |
| SEQ ID NO: 1071 | R0252:E06_3 | frame 3 | from 35 to 142 |
| SEQ ID NO: 1072 | R0252:E06_4 | frame −2 | from 79 to 142 |
| SEQ ID NO: 1073 | R0252:E06_5 | frame −3 | from 9 to 97 |
| SEQ ID NO: 1074 | R0252:E07_1 | frame 1 | from 1 to 59 |
| SEQ ID NO: 1075 | R0252:E07_2 | frame 2 | from 57 to 107 |
| SEQ ID NO: 1076 | R0252:E07_3 | frame 2 | from 109 to 184 |
| SEQ ID NO: 1077 | R0252:E07_4 | frame 3 | from 35 to 183 |
| SEQ ID NO: 1078 | R0252:E07_5 | frame −1 | from 51 to 139 |
| SEQ ID NO: 1079 | R0252:E07_6 | frame −3 | from 28 to 89 |
| SEQ ID NO: 1080 | R0252:E07_7 | frame −3 | from 120 to 183 |
| SEQ ID NO: 1081 | R0252:F11_1 | frame 1 | from 1 to 94 |
| SEQ ID NO: 1082 | R0252:F11_2 | frame 3 | from 1 to 61 |
| SEQ ID NO: 1083 | R0252:F11_3 | frame −2 | from 12 to 69 |
| SEQ ID NO: 1084 | R0252:F11_4 | frame −3 | from 1 to 139 |
| SEQ ID NO: 1085 | R0252:F02_1 | frame 1 | from 1 to 66 |
| SEQ ID NO: 1086 | R0252:F02_2 | frame 2 | from 57 to 107 |
| SEQ ID NO: 1087 | R0252:F02_3 | frame 2 | from 109 to 160 |
| SEQ ID NO: 1088 | R0252:F02_4 | frame 3 | from 35 to 159 |
| SEQ ID NO: 1089 | R0252:F02_5 | frame −1 | from 27 to 115 |
| SEQ ID NO: 1090 | R0252:F02_6 | frame −3 | from 4 to 65 |
| SEQ ID NO: 1091 | R0252:F02_7 | frame −3 | from 96 to 159 |
| SEQ ID NO: 1092 | R0252:H01_1 | frame 2 | from 1 to 123 |
| SEQ ID NO: 1093 | R0252:H01_2 | frame −3 | from 53 to 123 |
| SEQ ID NO: 1094 | R0252:H03_1 | frame 3 | from 1 to 85 |
| SEQ ID NO: 1095 | R0252:H03_2 | frame −3 | from 1 to 123 |
| SEQ ID NO: 1096 | R0253:B04_1 | frame 3 | from 1 to 85 |
| SEQ ID NO: 1097 | R0253:B04_2 | frame −3 | from 1 to 102 |
| SEQ ID NO: 1098 | R0253:C04_1 | frame 3 | from 1 to 85 |
| SEQ ID NO: 1099 | R0253:C04_2 | frame −2 | from 1 to 108 |
| SEQ ID NO: 1100 R0253:C05_1 | frame 3 | from 1 to 56 | |
| SEQ ID NO: 1101 | R0253:C05_2 | frame −1 | from 1 to 54 |
| SEQ ID NO: 1102 | R0253:C05_3 | frame −2 | from 9 to 63 |
| SEQ ID NO: 1103 | R0253:C06_1 | frame 3 | from 1 to 56 |
| SEQ ID NO: 1104 | R0253:D02_1 | frame 3 | from 1 to 55 |
| SEQ ID NO: 1105 | R0253:D02_2 | frame −3 | from 1 to 123 |
| SEQ ID NO: 1106 | R0253:D08_1 | frame 2 | from 1 to 194 |
| SEQ ID NO: 1107 | R0253:D08_2 | frame 3 | from 102 to 153 |
| SEQ ID NO: 1108 | R0253:D08_3 | frame −1 | from 1 to 55 |
| SEQ ID NO: 1109 | R0253:D08_4 | frame −1 | from 117 to 182 |
| SEQ ID NO: 1110 | R0253:D08_5 | frame −3 | from 30 to 88 |
| SEQ ID NO: 1111 | R0253:D08_6 | frame −3 | from 90 to 149 |
| SEQ ID NO: 1112 | R0253:E06_1 | frame 1 | from 1 to 51 |
| SEQ ID NO: 1113 | R0253:E06_2 | frame 2 | from 1 to 51 |
| SEQ ID NO: 1114 | R0253:F11_1 | frame 1 | from 1 to 79 |
| SEQ ID NO: 1115 | R0253:F11_2 | frame 3 | from 26 to 79 |
| SEQ ID NO: 1116 | R0253:F11_3 | frame −3 | from 1 to 59 |
| SEQ ID NO: 1117 | R0253:F07_1 | frame 3 | from 1 to 85 |
| SEQ ID NO: 1118 | R0253:F07_2 | frame −3 | from 1 to 93 |
| SEQ ID NO: 1119 | R0253:G11_1 | frame 2 | from 1 to 194 |
| SEQ ID NO: 1120 | R0253:G11_2 | frame 3 | from 102 to 153 |
| SEQ ID NO: 1121 | R0253:G11_3 | frame −1 | from 1 to 55 |
| SEQ ID NO: 1122 | R0253:G11_4 | frame −1 | from 117 to 182 |
| SEQ ID NO: 1123 | R0253:G11_5 | frame −3 | from 30 to 88 |
| SEQ ID NO: 1124 | R0253:G11_6 | frame −3 | from 90 to 149 |
| SEQ ID NO: 1125 | R0253:G12_1 | frame 1 | from 1 to 94 |
| SEQ ID NO: 1126 | R0253:G12_2 | frame 3 | from 1 to 53 |
| SEQ ID NO: 1127 | R0253:G05_1 | frame 3 | from 53 to 123 |
| SEQ ID NO: 1128 | R0253:G05_2 | frame −2 | from 1 to 124 |
| SEQ ID NO: 1129 | R0253:H02_1 | frame 2 | from 1 to 63 |
| SEQ ID NO: 1130 | R0253:H07_1 | frame 2 | from 1 to 73 |
| SEQ ID NO: 1131 | R0253:H07_2 | frame 3 | from 1 to 57 |
| SEQ ID NO: 1132 | R0254:F07_1 | frame 1 | from 69 to 153 |
| SEQ ID NO: 1133 | R0254:F07_2 | frame −1 | from 87 to 142 |
| SEQ ID NO: 1134 | R0254:F07_3 | frame −2 | from 47 to 116 |
| SEQ ID NO: 1135 | R0254:F07_4 | frame −3 | from 1 to 82 |
| SEQ ID NO: 1136 | R0254:F07_5 | frame −3 | from 99 to 154 |
| SEQ ID NO: 1137 | R0254:G11_1 | frame 3 | from 1 to 85 |
| SEQ ID NO: 1138 | R0254:G11_2 | frame −3 | from 1 to 123 |
| SEQ ID NO: 1139 | R0254:G04_1 | frame 3 | from 1 to 123 |
| SEQ ID NO: 1140 | R0254:G04_2 | frame −3 | from 1 to 85 |
| SEQ ID NO: 1141 | R0254:H01_1 | frame 3 | from 1 to 85 |
| SEQ ID NO: 1142 | R0254:H01_2 | frame −3 | from 1 to 123 |
| SEQ ID NO: 1143 | R0238:C03_1 | frame 2 | from 6 to 120 |
| SEQ ID NO: 1144 | R0238:C03_2 | frame 3 | from 103 to 157 |
| SEQ ID NO: 1145 | R0238:C03_3 | frame −1 | from 28 to 78 |
| SEQ ID NO: 1146 | R0255:C02_1 | frame 1 | from 1 to 60 |
| SEQ ID NO: 1147 | R0255:C02_2 | frame 3 | from 23 to 96 |
| SEQ ID NO: 1148 | R0255:C02_3 | frame −1 | from 35 to 108 |
| SEQ ID NO: 1149 | R0255:F12_1 | frame 3 | from 1 to 57 |
| SEQ ID NO: 1150 | R0255:F12_2 | frame −2 | from 1 to 78 |
| SEQ ID NO: 1151 | R0258:G10_1 | frame 1 | from 7 to 121 |
| SEQ ID NO: 1152 | R0258:G10_2 | frame 2 | from 104 to 158 |
| SEQ ID NO: 1153 | R0258:G10_3 | frame −1 | from 34 to 84 |
| SEQ ID NO: 1154 | R0261:A12_1 | frame 2 | from 2 to 60 |
| SEQ ID NO: 1155 | R0261:A12_2 | frame 3 | from 1 to 110 |
| SEQ ID NO: 1156 | R0261:A12_3 | frame −1 | from 1 to 145 |
| SEQ ID NO: 1157 | R0261:A12_4 | frame −3 | from 13 to 144 |
| SEQ ID NO: 1158 | R0261:A09_1 | frame 1 | from 1 to 174 |
| SEQ ID NO: 1159 | R0261:A09_2 | frame 2 | from 34 to 89 |
| SEQ ID NO: 1160 | R0261:A09_3 | frame 3 | from 1 to 52 |
| SEQ ID NO: 1161 | R0261:A09_4 | frame −1 | from 121 to 174 |
| SEQ ID NO: 1162 | R0261:A09_5 | frame −2 | from 47 to 116 |
| SEQ ID NO: 1163 | R0261:A09_6 | frame −2 | from 125 to 174 |
| SEQ ID NO: 1164 | R0261:A09_7 | frame −3 | from 32 to 174 |
| SEQ ID NO: 1165 | R0261:B12_1 | frame 1 | from 51 to 113 |
| SEQ ID NO: 1166 | R0261:B12_2 | frame −2 | from 2 to 51 |
| SEQ ID NO: 1167 | R0261:C10_1 | frame 2 | from 6 to 120 |
| SEQ ID NO: 1168 | R0261:C10_2 | frame 3 | from 103 to 157 |
| SEQ ID NO: 1169 | R0261:C10_3 | frame −1 | from 25 to 75 |
| SEQ ID NO: 1170 | R0261:D06_1 | frame 2 | from 1 to 117 |
| SEQ ID NO: 1171 | R0261:D06_2 | frame −2 | from 1 to 117 |
| SEQ ID NO: 1172 | R0261:D06_3 | frame −3 | from 35 to 117 |
| SEQ ID NO: 1173 | R0261:E04_1 | frame 2 | from 1 to 170 |
| SEQ ID NO: 1174 | R0261:E04_2 | frame −2 | from 32 to 122 |
| SEQ ID NO: 1175 | R0261:E04_3 | frame −3 | from 36 to 144 |
| SEQ ID NO: 1176 | R0261:F05_1 | frame 2 | from 61 to 111 |
| SEQ ID NO: 1177 | R0261:F05_2 | frame −1 | from 1 to 78 |
| SEQ ID NO: 1178 | R0261:F05_3 | frame −1 | from 105 to 157 |
| SEQ ID NO: 1179 | R0261:F05_4 | frame −2 | from 61 to 115 |
| SEQ ID NO: 1180 | R0261:G04_1 | frame 2 | from 13 to 111 |
| SEQ ID NO: 1181 | R0261:G04_2 | frame 3 | from 91 to 147 |
| SEQ ID NO: 1182 | R0261:G04_3 | frame −1 | from 83 to 169 |
| SEQ ID NO: 1183 | R0261:G04_4 | frame −2 | from 4 to 56 |
| SEQ ID NO: 1184 | R0261:G04_5 | frame −3 | from 123 to 181 |
| SEQ ID NO: 1185 | R0261:H03_1 | frame 2 | from 6 to 120 |
| SEQ ID NO: 1186 | R0261:H03_2 | frame 3 | from 103 to 157 |
| SEQ ID NO: 1187 | R0261:H03_3 | frame −1 | from 33 to 83 |
| SEQ ID NO: 1188 | R0262:A12_1 | frame −1 | from 35 to 132 |
| SEQ ID NO: 1189 | R0262:A02_1 | frame 1 | from 1 to 142 |
| SEQ ID NO: 1190 | R0262:A02_2 | frame 2 | from 18 to 81 |
| SEQ ID NO: 1191 | R0262:A02_3 | frame 3 | from 1 to 86 |
| SEQ ID NO: 1192 | R0262:A02_4 | frame −2 | from 1 to 73 |
| SEQ ID NO: 1193 | R0262:A02_5 | frame −3 | from 1 to 52 |
| SEQ ID NO: 1194 | R0262:D12_1 | frame 2 | from 4 to 118 |
| SEQ ID NO: 1195 | R0262:D04_1 | frame 1 | from 26 to 95 |
| SEQ ID NO: 1196 | R0262:D04_2 | frame 3 | from 32 to 94 |
| SEQ ID NO: 1197 | R0262:D04_3 | frame −2 | from 16 to 65 |
| SEQ ID NO: 1198 | R0262:D04_4 | frame −3 | from 1 to 92 |
| SEQ ID NO: 1199 | R0262:D07_1 | frame 1 | from 102 to 156 |
| SEQ ID NO: 1200 | R0262:D07_2 | frame 3 | from 4 to 118 |
| SEQ ID NO: 1201 | R0262:D07_3 | frame −1 | from 20 to 70 |
| SEQ ID NO: 1202 | R0262:E02_1 | frame 1 | from 7 to 121 |
| SEQ ID NO: 1203 | R0262:E02_2 | frame 2 | from 104 to 158 |
| SEQ ID NO: 1204 | R0262:E02_3 | frame −2 | from 27 to 77 |
| SEQ ID NO: 1205 | R0262:E03_1 | frame 1 | from 127 to 176 |
| SEQ ID NO: 1206 | R0262:E03_2 | frame 2 | from 26 to 159 |
| SEQ ID NO: 1207 | R0262:E03_3 | frame 3 | from 1 to 67 |
| SEQ ID NO: 1208 | R0262:E03_4 | frame −1 | from 9 to 68 |
| SEQ ID NO: 1209 | R0262:E03_5 | frame −2 | from 113 to 176 |
| SEQ ID NO: 1210 | R0262:E03_6 | frame −3 | from 107 to 159 |
| SEQ ID NO: 1211 | R0262:F06_1 | frame 1 | from 1 to 99 |
| SEQ ID NO: 1212 | R0262:F06_2 | frame 3 | from 13 to 98 |
| SEQ ID NO: 1213 | R0262:F06_3 | frame −2 | from 1 to 64 |
| SEQ ID NO: 1214 | R0263:B03_1 | frame 1 | from 1 to 84 |
| SEQ ID NO: 1215 | R0263:B03_2 | frame 3 | from 13 to 83 |
| SEQ ID NO: 1216 | R0263:B09_1 | frame 2 | from 1 to 199 |
| SEQ ID NO: 1217 | R0263:B09_2 | frame −1 | from 1 to 76 |
| SEQ ID NO: 1218 | R0263:B09_3 | frame −1 | from 78 to 199 |
| SEQ ID NO: 1219 | R0263:B09_4 | frame −2 | from 140 to 195 |
| SEQ ID NO: 1220 | R0263:E03_1 | frame 3 | from 50 to 111 |
| SEQ ID NO: 1221 | R0263:E03_2 | frame −1 | from 119 to 204 |
| SEQ ID NO: 1222 | R0263:F08_1 | frame 3 | from 1 to 95 |
| SEQ ID NO: 1223 | R0263:G10_1 | frame 1 | from 7 to 121 |
| SEQ ID NO: 1224 | R0263:G10_2 | frame 1 from 148 to 198 | |
| SEQ ID NO: 1225 | R0263:G10_3 | frame 2 | from 14 to 77 |
| SEQ ID NO: 1226 | R0263:G10_4 | frame 2 | from 104 to 158 |
| SEQ ID NO: 1227 | R0263:G10_5 | frame −1 | from 37 to 87 |
| SEQ ID NO: 1228 | R0263:G02_1 | frame 1 | from 54 to 126 |
| SEQ ID NO: 1229 | R0263:G02_2 | frame 2 | from 1 to 70 |
| SEQ ID NO: 1230 | R0263:G02_3 | frame −2 | from 109 to 190 |
| SEQ ID NO: 1231 | R0263:G02_4 | frame −3 | from 34 to 105 |
| SEQ ID NO: 1232 | R0263:G03_1 | frame 1 | from 90 to 139 |
| SEQ ID NO: 1233 | R0263:G03_2 | frame 2 | from 13 to 106 |
| SEQ ID NO: 1234 | R0263:G03_3 | frame −1 | from 3 to 55 |
| SEQ ID NO: 1235 | R0263:G03_4 | frame −2 | from 122 to 180 |
| SEQ ID NO: 1236 | R0263:G03_5 | frame −3 | from 77 to 167 |
| SEQ ID NO: 1237 | R0263:H10_1 | frame 1 | from 1 to 55 |
| SEQ ID NO: 1238 | R0263:H10_2 | frame 1 | from 99 to 152 |
| SEQ ID NO: 1239 | R0263:H10_3 | frame 3 | from 1 to 147 |
| SEQ ID NO: 1240 | R0263:H10_4 | frame −1 | from 6 to 140 |
| SEQ ID NO: 1241 | R0263:H10_5 | frame −3 | from 1 to 151 |
| SEQ ID NO: 1242 | R0264:A02_1 | frame 1 | from 1 to 85 |
| SEQ ID NO: 1243 | R0264:A02_2 | frame 3 | from 13 to 84 |
| SEQ ID NO: 1244 | R0264:A02_3 | frame −3 | from 1 to 50 |
| SEQ ID NO: 1245 | R0264:B11_1 | frame 2 | from 6 to 120 |
| SEQ ID NO: 1246 | R0264:B11_2 | frame 3 | from 103 to 157 |
| SEQ ID NO: 1247 | R0264:B11_3 | frame −1 | from 30 to 80 |
| SEQ ID NO: 1248 | R0264:E12_1 | frame 3 | from 50 to 111 |
| SEQ ID NO: 1249 | R0264:E12_2 | frame −1 | from 78 to 163 |
| SEQ ID NO: 1250 | R0264:F11_1 | frame 1 | from 13 to 81 |
| SEQ ID NO: 1251 | R0264:F11_2 | frame −1 | from 1 to 102 |
| SEQ ID NO: 1252 | R0264:F11_3 | frame −2 | from 25 to 101 |
| SEQ ID NO: 1253 | R0264:F11_4 | frame −3 | from 42 to 101 |
| SEQ ID NO: 1254 | R0264:F09_1 | frame 1 | from 7 to 121 |
| SEQ ID NO: 1255 | R0264:F09_2 | frame 2 | from 104 to 158 |
| SEQ ID NO: 1256 | R0264:F09_3 | frame −3 | from 25 to 75 |
| SEQ ID NO: 1257 | R0264:G01_1 | frame 2 | from 61 to 124 |
| SEQ ID NO: 1258 | R0264:G01_2 | frame 3 | from 24 to 82 |
| SEQ ID NO: 1259 | R0264:G01_3 | frame −1 | from 80 to 150 |
| SEQ ID NO: 1260 | R0264:G01_4 | frame −2 | from 1 to 94 |
| SEQ ID NO: 1261 | R0264:G11_1 | frame 1 | from 1 to 164 |
| SEQ ID NO: 1262 | R0264:G11_2 | frame 2 | from 74 to 145 |
| SEQ ID NO: 1263 | R0264:G11_3 | frame −2 | from 1 to 120 |
| SEQ ID NO: 1264 | R0264:G11_4 | frame −3 | from 50 to 163 |
| SEQ ID NO: 1265 | R0264:G04_1 | frame 2 | from 6 to 93 |
| SEQ ID NO: 1266 | R0265:F07_1 | frame 1 | from 1 to 75 |
| SEQ ID NO: 1267 | R0265:F07_2 | frame 1 | from 102 to 154 |
| SEQ ID NO: 1268 | R0265:F07_3 | frame 2 | from 58 to 112 |
| SEQ ID NO: 1269 | R0265:F07_4 | frame 2 | from 116 to 167 |
| SEQ ID NO: 1270 | R0265:F07_5 | frame −2 | from 61 to 111 |
| SEQ ID NO: 1271 | R0265:G01_1 | frame 1 | from 1 to 112 |
| SEQ ID NO: 1272 | R0265:G01_2 | frame 3 | from 3 to 61 |
| SEQ ID NO: 1273 | R0265:G01_3 | frame −1 | from 1 to 146 |
| SEQ ID NO: 1274 | R0265:G01_4 | frame −3 | from 13 to 146 |
| SEQ ID NO: 1275 | R0265:G10_1 | frame 1 | from 1 to 115 |
| SEQ ID NO: 1276 | R0265:G10_2 | frame 3 | from 13 to 114 |
| SEQ ID NO: 1277 | R0265:G10_3 | frame −2 | from 1 to 80 |
| SEQ ID NO: 1278 | R0265:G11_1 | frame 1 | from 59 to 122 |
| SEQ ID NO: 1279 | R0265:G11_2 | frame 3 | from 25 to 103 |
| SEQ ID NO: 1280 | R0265:G11_3 | frame −1 | from 14 to 91 |
| SEQ ID NO: 1281 | R0265:H09_1 | frame 1 | from 1 to 191 |
| SEQ ID NO: 1282 | R0265:H09_2 | frame −1 | from 1 to 51 |
| SEQ ID NO: 1283 | R0265:H09_3 | frame −1 | from 91 to 141 |
| SEQ ID NO: 1284 | R0265:H09_4 | frame −2 | from 98 to 152 |
| SEQ ID NO: 1285 | R0266:A11_1 | frame 1 | from 1 to 107 |
| SEQ ID NO: 1286 | R0266:A11_2 | frame 3 | from 1 to 56 |
| SEQ ID NO: 1287 | R0266:A11_3 | frame −1 | from 1 to 141 |
| SEQ ID NO: 1288 | R0266:A11_4 | frame −3 | from 13 to 125 |
| SEQ ID NO: 1289 | R0266:A12_1 | frame 1 | from 1 to 106 |
| SEQ ID NO: 1290 | R0266:A12_2 | frame 1 from 133 to 185 | |
| SEQ ID NO: 1291 | R0266:A12_3 | frame 2 | from 89 to 143 |
| SEQ ID NO: 1292 | R0266:A12_4 | frame 2 | from 147 to 197 |
| SEQ ID NO: 1293 | R0266:A12_5 | frame −3 | from 51 to 101 |
| SEQ ID NO: 1294 | R0266:B01_1 | frame 1 | from 20 to 93 |
| SEQ ID NO: 1295 | R0266:B01_2 | frame 2 | from 1 to 56 |
| SEQ ID NO: 1296 | R0266:B01_3 | frame −3 | from 31 to 104 |
| SEQ ID NO: 1297 | R0266:C12_1 | frame 1 | from 7 to 121 |
| SEQ ID NO: 1298 | R0266:C12_2 | frame 1 | from 148 to 200 |
| SEQ ID NO: 1299 | R0266:C12_3 | frame 2 | from 104 to 158 |
| SEQ ID NO: 1300 | R0266:C12_4 | frame −3 | from 41 to 93 |
| SEQ ID NO: 1301 | R0266:E01_1 | frame 3 | from 1 to 125 |
| SEQ ID NO: 1302 | R0266:E01_2 | frame −1 | from 75 to 133 |
| SEQ ID NO: 1303 | R0266:E01_3 | frame −2 | from 34 to 133 |
| SEQ ID NO: 1304 | R0266:E03_1 | frame 3 | from 81 to 130 |
| SEQ ID NO: 1305 | R0266:E03_2 | frame −1 | from 1 to 131 |
| SEQ ID NO: 1306 | R0266:E03_3 | frame −3 | from 1 to 53 |
| SEQ ID NO: 1307 | R0266:F03_1 | frame 1 | from 64 to 141 |
| SEQ ID NO: 1308 | R0266:F03_2 | frame 2 | from 8 to 141 |
| SEQ ID NO: 1309 | R0266:F03_3 | frame 3 | from 39 to 104 |
| SEQ ID NO: 1310 | R0266:F03_4 | frame −2 | from 1 to 141 |
| SEQ ID NO: 1311 | R0266:F07_1 | frame −3 | from 37 to 97 |
| SEQ ID NO: 1312 | R0266:F07_2 | frame −3 | from 138 to 188 |
| SEQ ID NO: 1313 | R0266:G10_1 | frame 3 | from 24 to 124 |
| SEQ ID NO: 1314 | R0266:G10_2 | frame −2 | from 1 to 150 |
| SEQ ID NO: 1315 | R0266:G09_1 | frame 1 | from 7 to 121 |
| SEQ ID NO: 1316 | R0266:G09_2 | frame 2 | from 104 to 158 |
| SEQ ID NO: 1317 | R0266:G09_3 | frame −2 | from 28 to 78 |
| SEQ ID NO: 1318 | R0266:H09_1 | frame 1 | from 1 to 68 |
| SEQ ID NO: 1319 | R0266:H09_2 | frame 3 | from 48 to 148 |
| SEQ ID NO: 1320 | R0266:H09_3 | frame −2 | from 1 to 137 |
| SEQ ID NO: 1321 | R0243:F07_1 | frame 1 | from 19 to 77 |
| SEQ ID NO: 1322 | R0243:F07_2 | frame 2 | from 13 to 76 |
| SEQ ID NO: 1323 | R0243:F07_3 | frame 3 | from 20 to 76 |
| SEQ ID NO: 1324 | R0243:F07_4 | frame −1 | from 15 to 65 |
| SEQ ID NO: 1325 | R0244:C02_1 | frame 1 | from 1 to 64 |
| SEQ ID NO: 1326 | R0244:C02_2 | frame −1 | from 8 to 107 |
| SEQ ID NO: 1327 | R0244:C02_3 | frame −2 | from 19 to 70 |
| SEQ ID NO: 1328 | R0244:C04_1 | frame 1 | from 19 to 77 |
| SEQ ID NO: 1329 | R0244:C04_2 | frame 2 | from 13 to 76 |
| SEQ ID NO: 1330 | R0244:C04_3 | frame 3 | from 20 to 76 |
| SEQ ID NO: 1331 | R0244:C04_4 | frame −1 | from 15 to 65 |
| SEQ ID NO: 1332 | R0245:A02_1 | frame 2 | from 12 to 61 |
| SEQ ID NO: 1333 | R0245:A02_2 | frame −3 | from 42 to 92 |
| SEQ ID NO: 1334 | ‘46802.1_gaiger.ABI’_1 | frame 1 | from 1 to 90 |
| SEQ ID NO: 1335 | ‘46802.1_gaiger.ABI’_2 | frame −1 | from 1 to 52 |
| SEQ ID NO: 1336 | ‘46816.1_gaiger.ABI’_1 | frame 2 | from 1 to 166 |
| SEQ ID NO: 1337 | ‘46816.1_gaiger.ABI’_2 | frame −2 | from 16 to 91 |
| SEQ ID NO: 1338 | ‘46816.1_gaiger.ABI’_3 | frame −2 | from 94 to 166 |
| SEQ ID NO: 1339 | ‘46816.1_gaiger.ABI’_4 | frame −3 | from 99 to 166 |
| SEQ ID NO: 1340 | ‘46880.1_gaiger.ABI’_1 | frame 2 | from 36 to 95 |
| SEQ ID NO: 1341 | ‘46880.1_gaiger.ABI’_2 | frame 3 from 1 to 95 | |
| SEQ ID NO: 1342 | ‘46880.1_gaiger.ABI’_3 | frame −1 | from 32 to 81 |
| SEQ ID NO: 1343 | ‘47011.1_gaiger.ABI’_1 | frame 1 | from 1 to 102 |
| SEQ ID NO: 1344 | ‘47011.1_gaiger.ABI’_2 | frame 3 | from 42 to 101 |
| SEQ ID NO: 1345 | ‘47011.1_gaiger.ABI’_3 | frame −1 | from 32 to 81 |
| SEQ ID NO: 1346 | ‘51658.1_gaiger.ABI’_1 | frame 2 | from 5 to 80 |
| SEQ ID NO: 1347 | ‘51658.1_gaiger.ABI’_2 | frame 3 | from 10 to 77 |
| SEQ ID NO: 1348 | ‘51734.1_gaiger.ABI’_1 | frame 1 | from 12 to 98 |
| SEQ ID NO: 1349 | ‘51734.1_gaiger.ABI’_2 | frame 3 | from 22 to 76 |
| SEQ ID NO: 1350 | ‘51734.1_gaiger.ABI’_3 | frame −2 | from 18 to 137 |
| SEQ ID NO: 1351 | ‘51735.1_gaiger.ABI’_1 | frame 1 | from 30 to 153 |
| SEQ ID NO: 1352 | ‘51735.1_gaiger.ABI’_2 | frame 3 | from 1 to 69 |
| SEQ ID NO: 1353 | ‘51735.1_gaiger.ABI’_3 | frame −2 | from 44 to 123 |
| SEQ ID NO: 1354 | ‘51788.1_gaiger.ABI’_1 | frame 1 | from 1 to 59 |
| SEQ ID NO: 1355 | ‘51788.1_gaiger.ABI’_2 | frame −2 | from 1 to 68 |
| SEQ ID NO: 1356 | ‘51892.1_gaiger.ABI’_1 | frame 1 | from 1 to 158 |
| SEQ ID NO: 1357 | ‘51892.1_gaiger.ABI’_2 | frame 2 | from 2 to 69 |
| SEQ ID NO: 1358 | ‘51892.1_gaiger.ABI’_3 | frame −1 | from 76 to 139 |
| SEQ ID NO: 1359 | ‘51892.1_gaiger.ABI’_4 | frame −2 | from 35 to 137 |
| SEQ ID NO: 1360 | ‘51900.1_gaiger.ABI’_1 | frame 2 | from 1 to 123 |
| SEQ ID NO: 1361 | ‘51900.1_gaiger.ABI’_2 | frame 3 | from 3 to 70 |
| SEQ ID NO: 1362 | ‘51900.1_gaiger.ABI’_3 | frame −2 | from 78 to 141 |
| SEQ ID NO: 1363 | ‘51900.1_gaiger.ABI’_4 | frame −3 | from 36 to 139 |
| SEQ ID NO: 1364 | 1404:D07_1 | frame 1 | from 3 to 150 |
| SEQ ID NO: 1365 | 1404:D07_2 | frame 2 | from 8 to 75 |
| SEQ ID NO: 1366 | 1404:D07_3 | frame −1 | from 13 to 115 |
| SEQ ID NO: 1367 | 1404:D07_4 | frame −3 | from 53 to 116 |
| SEQ ID NO: 1368 | 1405:C04_1 | frame 2 | from 1 to 50 |
| SEQ ID NO: 1369 | 1405:C04_2 | frame 3 | from 10 to 102 |
| SEQ ID NO: 1370 | 1405:C04_3 | frame −2 | from 76 to 140 |
| SEQ ID NO: 1371 | 1405:D12_1 | frame 1 | from 4 to 71 |
| SEQ ID NO: 1372 | 1405:D12_2 | frame 3 | from 1 to 143 |
| SEQ ID NO: 1373 | 1405:D12_3 | frame −1 | from 52 to 115 |
| SEQ ID NO: 1374 | 1405:D12_4 | frame −2 | from 11 to 113 |
| SEQ ID NO: 1375 | 1405:E11_1 | frame 1 | from 87 to 159 |
| SEQ ID NO: 1376 | 1405:E11_2 | frame 3 | from 92 to 143 |
| SEQ ID NO: 1377 | 1405:E11_3 | frame −2 | from 48 to 111 |
| SEQ ID NO: 1378 | 1405:E11_4 | frame −3 | from 1 to 55 |
| SEQ ID NO: 1379 | ‘52333.1_gaiger.ABI’_1 | frame 1 | from 1 to 69 |
| SEQ ID NO: 1380 | ‘52333.1_gaiger.ABI’_2 | frame 2 from 1 to 66 | |
| SEQ ID NO: 1381 | ‘41557.1_gaiger.ABI’_1 | frame 1 | from 16 to 73 |
| SEQ ID NO: 1382 | ‘41557.1_gaiger.ABI’_2 | frame 2 | from 1 to 109 |
| SEQ ID NO: 1383 | ‘41557.1_gaiger.ABI’_3 | frame −1 | from 11 to 110 |
| SEQ ID NO: 1384 | ‘41557.1_gaiger.ABI’_4 | frame −3 | from 1 to 103 |
| SEQ ID NO: 1385 | ‘41579.1_gaiger.ABI’_1 | frame 3 | from 43 to 97 |
| SEQ ID NO: 1386 | ‘41579.1_gaiger.ABI’_2 | frame −2 | from 1 to 97 |
| SEQ ID NO: 1387 | ‘41579.1_gaiger.ABI’_3 | frame −3 | from 43 to 97 |
| SEQ ID NO: 1388 | ‘41571.1_gaiger.ABI’_1 | frame 3 | from 1 to 89 |
| SEQ ID NO: 1389 | ‘41571.1_gaiger.ABI’_2 | frame −1 | from 1 to 89 |
| SEQ ID NO: 1390 | ‘41571.1_gaiger.ABI’_3 | frame −2 | from 27 to 85 |
| SEQ ID NO: 1391 | ‘41613.1_gaiger.ABI’_1 | frame 3 | from 1 to 136 |
| SEQ ID NO: 1392 | ‘41613.1_gaiger.ABI’_2 | frame −1 | from 40 to 163 |
| SEQ ID NO: 1393 | ‘41613.1_gaiger.ABI’_3 | frame −2 | from 49 to 100 |
| SEQ ID NO: 1394 | ‘41613.1_gaiger.ABI’_4 | frame −3 | from 3 to 61 |
| SEQ ID NO: 1395 | ‘41650.1_gaiger.ABI’_1 | frame 1 | from 22 to 109 |
| SEQ ID NO: 1396 | ‘41650.1_gaiger.ABI’_2 | frame 2 | from 1 to 157 |
| SEQ ID NO: 1397 | ‘41650.1_gaiger.ABI’_3 | frame 3 | from 1 to 156 |
| SEQ ID NO: 1398 | ‘41650.1_gaiger.ABI’_4 | frame −1 | from 25 to 99 |
| SEQ ID NO: 1399 | ‘41650.1_gaiger.ABI’_5 | frame −2 | from 47 to 157 |
| SEQ ID NO: 1400 | ‘41650.1_gaiger.ABI’_6 | frame −3 | from 53 to 156 |
| SEQ ID NO: 1401 | ‘41663.1_gaiger.ABI’_1 | frame −2 | from 64 to 116 |
| SEQ ID NO: 1402 | ‘41663.1_gaiger.ABI’_2 | frame −3 | from 1 to 67 |
| SEQ ID NO: 1403 | ‘41687.1_gaiger.ABI’_1 | frame 1 | from 35 to 154 |
| SEQ ID NO: 1404 | ‘41687.1_gaiger.ABI’_2 | frame 2 | from 102 to 153 |
| SEQ ID NO: 1405 | ‘41687.1_gaiger.ABI’_3 | frame −1 | from 50 to 109 |
| SEQ ID NO: 1406 | ‘41687.1_gaiger.ABI’_4 | frame −3 | from 102 to 153 |
| SEQ ID NO: 1407 | ‘41717.1_gaiger.ABI’_1 | frame 1 | from 55 to 129 |
| SEQ ID NO: 1408 | ‘41717.1_gaiger.ABI’_2 | frame 2 | from 1 to 63 |
| SEQ ID NO: 1409 | ‘41717.1_gaiger.ABI’_3 | frame −3 | from 1 to 68 |
| SEQ ID NO: 1410 | ‘41751.1_gaiger.ABI’_1 | frame 1 | from 27 to 82 |
| SEQ ID NO: 1411 | ‘41751.1_gaiger.ABI’_2 | frame 3 | from 1 to 50 |
| SEQ ID NO: 1412 | ‘41751.1_gaiger.ABI’_3 | frame −2 | from 1 to 70 |
| SEQ ID NO: 1413 | ‘41751.1_gaiger.ABI’_4 | frame −3 | from 1 to 53 |
| SEQ ID NO: 1414 | ‘41818.1_gaiger.ABI’_1 | frame 2 | from 1 to 69 |
| SEQ ID NO: 1415 | ‘41818.1_gaiger.ABI’_2 | frame −1 | from 30 to 93 |
| SEQ ID NO: 1416 | ‘41818.1_gaiger.ABI’_3 | frame −3 | from 1 to 92 |
| SEQ ID NO: 1417 | ‘41828.1_gaiger.ABI’_1 | frame −3 | from 1 to 77 |
| SEQ ID NO: 1418 | ‘41849.1_gaiger.ABI’_1 | frame 1 | from 1 to 75 |
| SEQ ID NO: 1419 | ‘41849.1_gaiger.ABI’_2 | frame 3 | from 4 to 77 |
| SEQ ID NO: 1420 | ‘41849.1_gaiger.ABI’_3 | frame −1 | from 12 to 77 |
| SEQ ID NO: 1421 | ‘41881.1_gaiger.ABI’_1 | frame −1 | from 1 to 127 |
| SEQ ID NO: 1422 | ‘41881.1_gaiger.ABI’_2 | frame −2 | from 73 to 126 |
| SEQ ID NO: 1423 | ‘41881.1_gaiger.ABI’_3 | frame −3 | from 1 to 76 |
| SEQ ID NO: 1424 | ‘41912.1_gaiger.ABI’_1 | frame 2 | from 1 to 138 |
| SEQ ID NO: 1425 | ‘41912.1_gaiger.ABI’_2 | frame −2 | from 34 to 93 |
| SEQ ID NO: 1426 | ‘41912.1_gaiger.ABI’_3 | frame −3 | from 60 to 125 |
| SEQ ID NO: 1427 | ‘41927.1_gaiger.ABI’_1 | frame 3 | from 20 to 74 |
| SEQ ID NO: 1428 | ‘41929.1_gaiger.ABI’_1 | frame 1 | from 1 to 52 |
| SEQ ID NO: 1429 | ‘41944.1_gaiger.ABI’_1 | frame 1 | from 1 to 56 |
| SEQ ID NO: 1430 | ‘41944.1_gaiger.ABI’_2 | frame 2 | from 1 to 177 |
| SEQ ID NO: 1431 | ‘41944.1_gaiger.ABI’_3 | frame 3 | from 37 to 92 |
| SEQ ID NO: 1432 | ‘41944.1_gaiger.ABI’_4 | frame −1 | from 47 to 116 |
| SEQ ID NO: 1433 | ‘41944.1_gaiger.ABI’_5 | frame −1 | from 125 to 177 |
| SEQ ID NO: 1434 | ‘41944.1_gaiger.ABI’_6 | frame −2 | from 32 to 177 |
| SEQ ID NO: 1435 | ‘41944.1_gaiger.ABI’_7 | frame −3 | from 120 to 177 |
| SEQ ID NO: 1436 | ‘41987.1_gaiger.ABI’_1 | frame 1 | from 48 to 116 |
| SEQ ID NO: 1437 | ‘41987.1_gaiger.ABI’_2 | frame 2 | from 1 to 50 |
| SEQ ID NO: 1438 | ‘41987.1_gaiger.ABI’_3 | frame 2 | from 96 to 154 |
| SEQ ID NO: 1439 | ‘41987.1_gaiger.ABI’_4 | frame 3 | from 53 to 120 |
| SEQ ID NO: 1440 | ‘41987.1_gaiger.ABI’_5 | frame 3 | from 122 to 175 |
| SEQ ID NO: 1441 | ‘41987.1_gaiger.ABI’_6 | frame −1 | from 37 to 136 |
| SEQ ID NO: 1442 | ‘41987.1_gaiger.ABI’_7 | frame −2 | from 1 to 72 |
| SEQ ID NO: 1443 | ‘41995.1_gaiger.ABI’_1 | frame 1 | from 1 to 115 |
| SEQ ID NO: 1444 | ‘41995.1_gaiger.ABI’_2 | frame 3 | from 60 to 109 |
| SEQ ID NO: 1445 | ‘41995.1_gaiger.ABI’_3 | frame −2 | from 1 to 114 |
| SEQ ID NO: 1446 | ‘41995.1_gaiger.ABI’_4 | frame −3 | from 35 to 108 |
| SEQ ID NO: 1447 | ‘42012.1_gaiger.ABI’_1 | frame 2 | from 1 to 60 |
| SEQ ID NO: 1448 | ‘42012.1_gaiger.ABI’_2 | frame −3 | from 1 to 60 |
| SEQ ID NO: 1449 | ‘42039.1_gaiger.ABI’_1 | frame 2 | from 70 to 127 |
| SEQ ID NO: 1450 | ‘42039.1_gaiger.ABI’_2 | frame 3 | from 1 to 146 |
| SEQ ID NO: 1451 | ‘42039.1_gaiger.ABI’_3 | frame −2 | from 39 to 100 |
| SEQ ID NO: 1452 | ‘42097.1_gaiger.ABI’_1 | frame 1 | from 24 to 132 |
| SEQ ID NO: 1453 | ‘42097.1_gaiger.ABI’_2 | frame −1 | from 52 to 132 |
| SEQ ID NO: 1454 | ‘42097.1_gaiger.ABI’_3 | frame −3 | from 34 to 92 |
| SEQ ID NO: 1455 | ‘42103.1_gaiger.ABI’_1 | frame 1 | from 1 to 153 |
| SEQ ID NO: 1456 | ‘42103.1_gaiger.ABI’_2 | frame 2 | from 24 to 83 |
| SEQ ID NO: 1457 | ‘42103.1_gaiger.ABI’_3 | frame 2 | from 85 to 182 |
| SEQ ID NO: 1458 | ‘42103.1_gaiger.ABI’_4 | frame −2 | from 27 to 99 |
| SEQ ID NO: 1459 | ‘42103.1_gaiger.ABI’_5 | frame −2 | from 113 to 174 |
| SEQ ID NO: 1460 | ‘42103.1_gaiger.ABI’_6 | frame −3 | from 38 to 126 |
| SEQ ID NO: 1461 | ‘42108.1_gaiger.ABI’_1 | frame −2 | from 4 to 77 |
| SEQ ID NO: 1462 | R0233:A06_1 | frame 1 | from 12 to 77 |
| SEQ ID NO: 1463 | R0233:A06_2 | frame 3 | from 2 to 76 |
| SEQ ID NO: 1464 | R0233:A06_3 | frame −3 | from 1 to 59 |
| SEQ ID NO: 1465 | R0233:A08_1 | frame 1 | from 1 to 59 |
| SEQ ID NO: 1466 | R0233:A08_2 | frame −1 | from 1 to 63 |
| SEQ ID NO: 1467 | R0233:C02_1 | frame 3 | from 26 to 90 |
| SEQ ID NO: 1468 | R0233:C02_2 | frame −2 | from 1 to 107 |
| SEQ ID NO: 1469 | R0233:C02_3 | frame −3 | from 1 to 74 |
| SEQ ID NO: 1470 | R0233:E06_1 | frame 1 | from 84 to 146 |
| SEQ ID NO: 1471 | R0233:E06_2 | frame 3 | from 1 to 181 |
| SEQ ID NO: 1472 | R0233:E06_3 | frame −2 | from 49 to 157 |
| SEQ ID NO: 1473 | R0233:F08_1 | frame 1 | from 11 to 110 |
| SEQ ID NO: 1474 | R0233:F08_2 | frame 3 | from 1 to 103 |
| SEQ ID NO: 1475 | R0233:F08_3 | frame −1 | from 16 to 73 |
| SEQ ID NO: 1476 | R0233:F08_4 | frame −2 | from 1 to 109 |
| SEQ ID NO: 1477 | ‘42324.1_gaiger.ABI’_1 | frame 1 | from 1 to 94 |
| SEQ ID NO: 1478 | ‘42324.1_gaiger.ABI’_2 | frame 2 | from 1 to 57 |
| SEQ ID NO: 1479 | ‘42324.1_gaiger.ABI’_3 | frame 3 from 38 to 130 | |
| SEQ ID NO: 1480 | ‘42324.1_gaiger.ABI’_4 | frame −1 | from 10 to 130 |
| SEQ ID NO: 1481 | ‘42324.1_gaiger.ABI’_5 | frame −2 | from 1 to 54 |
| SEQ ID NO: 1482 | ‘42324.1_gaiger.ABI’_6 | frame −2 | from 72 to 130 |
| SEQ ID NO: 1483 | ‘42324.1_gaiger.ABI’_7 | frame −3 | from 1 to 67 |
| SEQ ID NO: 1484 | ‘42324.1_gaiger.ABI’_8 | frame −3 | from 76 to 130 |
| SEQ ID NO: 1485 | ‘42469.1;gaiger.ABI’_1 | frame 3 | from 11 to 90 |
| SEQ ID NO: 1486 | ‘42514.1;gaiger.ABI’_1 | frame 2 | from 14 to 89 |
| SEQ ID NO: 1487 | ‘42514.1;gaiger.ABI’_2 | frame −2 | from 10 to 76 |
| SEQ ID NO: 1488 | ‘42554.1;gaiger.ABI’_1 | frame 1 | from 1 to 67 |
| SEQ ID NO: 1489 | ‘42554.1;gaiger.ABI’_2 | frame 2 | from 6 to 63 |
| SEQ ID NO: 1490 | ‘42554.1;gaiger.ABI’_3 | frame −1 | from 7 to 67 |
| SEQ ID NO: 1491 | ‘42554.1;gaiger.ABI’_4 | frame −2 | from 1 to 56 |
| SEQ ID NO: 1492 | ‘42560.1;gaiger.ABI’_1 | frame 1 | from 1 to 67 |
| SEQ ID NO: 1493 | ‘42560.1;gaiger.ABI’_2 | frame −3 | from 1 to 66 |
| SEQ ID NO: 1494 | ‘42588.1_gaiger.ABI’_1 | frame 1 | from 1 to 60 |
| SEQ ID NO: 1495 | ‘42588.1_gaiger.ABI’_2 | frame 2 from 1 to 60 | |
| SEQ ID NO: 1496 | ‘42588.1_gaiger.ABI’_3 | frame 3 | from 1 to 60 |
| SEQ ID NO: 1497 | ‘42588.1_gaiger.ABI’_4 | frame −1 | from 1 to 60 |
| SEQ ID NO: 1498 | ‘42588.1_gaiger.ABI’_5 | frame −2 | from 1 to 53 |
| SEQ ID NO: 1499 | ‘42609.1_gaiger.ABI’_1 | frame 1 | from 1 to 51 |
| SEQ ID NO: 1500 | ‘42609.1_gaiger.ABI’_2 | frame 2 | from 1 to 79 |
| SEQ ID NO: 1501 | ‘42609.1_gaiger.ABI’_3 | frame −1 | from 10 to 80 |
| SEQ ID NO: 1502 | ‘42609.1_gaiger.ABI’_4 | frame −3 | from 2 to 68 |
| SEQ ID NO: 1503 | ‘42703.1_gaiger.ABI’_1 | frame 2 | from 25 to 95 |
| SEQ ID NO: 1504 | ‘42703.1_gaiger.ABI’_2 | frame −2 | from 10 to 82 |
| SEQ ID NO: 1505 | R0234:E06_1 | frame 3 | from 4 to 77 |
| SEQ ID NO: 1506 | R0234:E06_2 | frame −1 | from 1 to 66 |
| SEQ ID NO: 1507 | R0235:A09_1 | frame 3 | from 1 to 98 |
| SEQ ID NO: 1508 | R0235:A09_2 | frame −1 | from 15 to 76 |
| SEQ ID NO: 1509 | R0235:A09_3 | frame −2 | from 2 to 98 |
| SEQ ID NO: 1510 | R0235:A09_4 | frame −3 | from 1 to 54 |
| SEQ ID NO: 1511 | R0235:D01_1 | frame 1 | from 1 to 137 |
| SEQ ID NO: 1512 | R0235:D01_2 | frame 3 | from 1 to 67 |
| SEQ ID NO: 1513 | R0235:D01_3 | frame −1 | from 1 to 137 |
| SEQ ID NO: 1514 | R0235:D01_4 | frame −2 | from 1 to 61 |
| SEQ ID NO: 1515 | R0236:D04_1 | frame 1 | from 1 to 87 |
| SEQ ID NO: 1516 | R0236:D04_2 | frame 2 | from 1 to 113 |
| SEQ ID NO: 1517 | R0236:D04_3 | frame −1 | from 1 to 79 |
| SEQ ID NO: 1518 | R0236:F10_1 | frame 1 | from 1 to 51 |
| SEQ ID NO: 1519 | R0236:F10_2 | frame 2 | from 1 to 79 |
| SEQ ID NO: 1520 | R0236:F10_3 | frame −1 | from 10 to 80 |
| SEQ ID NO: 1521 | R0236:F10_4 | frame −3 | from 1 to 68 |
| SEQ ID NO: 1522 | R0236:G10_1 | frame 2 | from 1 to 117 |
| SEQ ID NO: 1523 | R0236:G10_2 | frame −3 | from 42 to 109 |
| SEQ ID NO: 1524 | R0236:G08_1 | frame 2 | from 1 to 88 |
| SEQ ID NO: 1525 | R0236:G08_2 | frame 3 | from 34 to 88 |
| SEQ ID NO: 1526 | R0249:D01_1 | frame 1 | from 25 to 76 |
| SEQ ID NO: 1527 | R0249:D01_2 | frame 2 | from 1 to 75 |
| SEQ ID NO: 1528 | R0249:D01_3 | frame −1 | from 1 to 76 |
| SEQ ID NO: 1529 | R0249:D01_4 | frame −2 | from 1 to 52 |
| SEQ ID NO: 1530 | R0249:G04_1 | frame 1 | from 1 to 96 |
| SEQ ID NO: 1531 | R0249:G04_2 | frame 2 | from 30 to 83 |
| SEQ ID NO: 1532 | R0249:G04_3 | frame 3 | from 13 to 71 |
| SEQ ID NO: 1533 | R0249:G04_4 | frame 3 | from 120 to 174 |
| SEQ ID NO: 1534 | R0249:G04_5 | frame −3 | from 1 to 66 |
| SEQ ID NO: 1535 | R0250:A10_1 | frame 1 | from 127 to 180 |
| SEQ ID NO: 1536 | R0250:A10_2 | frame −1 | from 1 to 55 |
| SEQ ID NO: 1537 | R0250:A10_3 | frame −2 | from 20 to 80 |
| SEQ ID NO: 1538 | R0250:A10_4 | frame −3 | from 1 to 96 |
| SEQ ID NO: 1539 | R0250:E12_1 | frame 1 | from 1 to 115 |
| SEQ ID NO: 1540 | R0250:E12_2 | frame 3 | from 60 to 109 |
| SEQ ID NO: 1541 | R0250:E12_3 | frame −2 | from 1 to 114 |
| SEQ ID NO: 1542 | R0250:E12_4 | frame −3 | from 35 to 108 |
| SEQ ID NO: 1543 | R0250:F12_1 | frame 1 | from 1 to 55 |
| SEQ ID NO: 1544 | R0250:F12_2 | frame 2 | from 20 to 80 |
| SEQ ID NO: 1545 | R0250:F12_3 | frame 3 | from 1 to 96 |
| SEQ ID NO: 1546 | R0250:F12_4 | frame −1 | from 127 to 180 |
| SEQ ID NO: 1547 | R0251:B08_1 | frame 1 | from 121 to 172 |
| SEQ ID NO: 1548 | R0251:B08_2 | frame −2 | from 61 to 122 |
| SEQ ID NO: 1549 | R0251:B08_3 | frame −3 | from 9 to 70 |
| SEQ ID NO: 1550 | R0251:B08_4 | frame −3 | from 72 to 133 |
| SEQ ID NO: 1551 | R0252:A08_1 | frame 1 | from 1 to 64 |
| SEQ ID NO: 1552 | R0252:A08_2 | frame 2 | from 12 to 64 |
| SEQ ID NO: 1553 | R0252:A08_3 | frame −1 | from 1 to 51 |
| SEQ ID NO: 1554 | R0252:A08_4 | frame −2 | from 1 to 64 |
| SEQ ID NO: 1555 | R0252:F11_1 | frame 1 | from 1 to 94 |
| SEQ ID NO: 1556 | R0252:F11_2 | frame 3 | from 1 to 61 |
| SEQ ID NO: 1557 | R0252:F11_3 | frame −2 | from 12 to 69 |
| SEQ ID NO: 1558 | R0252:F11_4 | frame −3 | from 1 to 139 |
| SEQ ID NO: 1559 | R0252:F02_1 | frame 1 | from 1 to 66 |
| SEQ ID NO: 1560 | R0252:F02_2 | frame 2 | from 57 to 107 |
| SEQ ID NO: 1561 | R0252:F02_3 | frame 2 | from 109 to 160 |
| SEQ ID NO: 1562 | R0252:F02_4 | frame 3 | from 35 to 159 |
| SEQ ID NO: 1563 | R0252:F02_5 | frame −1 | from 27 to 115 |
| SEQ ID NO: 1564 | R0252:F02_6 | frame −3 | from 4 to 65 |
| SEQ ID NO: 1565 | R0252:F02_7 | frame −3 | from 96 to 159 |
| SEQ ID NO: 1566 | R0252:G11_1 | frame 1 | from 1 to 131 |
| SEQ ID NO: 1567 | R0252:G11_2 | frame 2 | from 51 to 105 |
| SEQ ID NO: 1568 | R0252:G11_3 | frame −1 | from 13 to 131 |
| SEQ ID NO: 1569 | R0252:G11_4 | frame −2 | from 61 to 113 |
| SEQ ID NO: 1570 | R0253:E10_1 | frame 2 | from 46 to 118 |
| SEQ ID NO: 1571 | R0253:E10_2 | frame −1 | from 84 to 139 |
| SEQ ID NO: 1572 | R0253:G11_1 | frame 2 | from 1 to 194 |
| SEQ ID NO: 1573 | R0253:G11_2 | frame 3 | from 102 to 153 |
| SEQ ID NO: 1574 | R0253:G11_3 | frame −1 | from 1 to 55 |
| SEQ ID NO: 1575 | R0253:G11_4 | frame −1 | from 117 to 182 |
| SEQ ID NO: 1576 | R0253:G11_5 | frame −3 | from 30 to 88 |
| SEQ ID NO: 1577 | R0253:G11_6 | frame −3 | from 90 to 149 |
| SEQ ID NO: 1578 | R0254:A08_1 | frame 3 | from 1 to 85 |
| SEQ ID NO: 1579 | R0254:A08_2 | frame −1 | from 47 to 98 |
| SEQ ID NO: 1580 | R0254:E04_1 | frame 2 | from 12 to 65 |
| SEQ ID NO: 1581 | R0254:E04_2 | frame 3 | from 49 to 135 |
| SEQ ID NO: 1582 | R0254:F07_1 | frame 1 | from 69 to 153 |
| SEQ ID NO: 1583 | R0254:F07_2 | frame −1 | from 87 to 142 |
| SEQ ID NO: 1584 | R0254:F07_3 | frame −2 | from 47 to 116 |
| SEQ ID NO: 1585 | R0254:F07_4 | frame −3 | from 1 to 82 |
| SEQ ID NO: 1586 | R0254:F07_5 | frame −3 | from 99 to 154 |
| SEQ ID NO: 1587 | R0237:F12_1 | frame 2 | from 64 to 115 |
| SEQ ID NO: 1588 | R0237:F12_2 | frame 3 | from 1 to 99 |
| SEQ ID NO: 1589 | R0237:F12_3 | frame −1 | from 1 to 145 |
| SEQ ID NO: 1590 | R0237:F12_4 | frame −2 | from 19 to 134 |
| SEQ ID NO: 1591 | R0238:B02_1 | frame 3 | from 50 to 111 |
| SEQ ID NO: 1592 | R0238:B02_2 | frame −2 | from 102 to 187 |
| SEQ ID NO: 1593 | R0239:H02_1 | frame 3 | from 1 to 97 |
| SEQ ID NO: 1594 | R0255:F12_1 | frame 3 | from 1 to 57 |
| SEQ ID NO: 1595 | R0255:F12_2 | frame −2 | from 1 to 78 |
| SEQ ID NO: 1596 | R0258:B10_1 | frame 2 | from 1 to 130 |
| SEQ ID NO: 1597 | R0258:B10_2 | frame 3 | from 1 to 73 |
| SEQ ID NO: 1598 | R0258:B10_3 | frame −2 | from 86 to 142 |
| SEQ ID NO: 1599 | R0258:B10_4 | frame −3 | from 1 to 69 |
| SEQ ID NO: 1600 | R0259:C06_1 | frame −1 | from 36 to 100 |
| SEQ ID NO: 1601 | R0259:C06_2 | frame −2 | from 124 to 187 |
| SEQ ID NO: 1602 | R0261:A09_1 | frame 1 | from 1 to 174 |
| SEQ ID NO: 1603 | R0261:A09_2 | frame 2 | from 34 to 89 |
| SEQ ID NO: 1604 | R0261:A09_3 | frame 3 | from 1 to 52 |
| SEQ ID NO: 1605 | R0261:A09_4 | frame −1 | from 121 to 174 |
| SEQ ID NO: 1606 | R0261:A09_5 | frame −2 | from 47 to 116 |
| SEQ ID NO: 1607 | R0261:A09_6 | frame −2 | from 125 to 174 |
| SEQ ID NO: 1608 | R0261:A09_7 | frame −3 | from 32 to 174 |
| SEQ ID NO: 1609 | R0261:B10_1 | frame 1 | from 1 to 79 |
| SEQ ID NO: 1610 | R0261:B10_2 | frame −1 | from 1 to 87 |
| SEQ ID NO: 1611 | R0261:B10_3 | frame −2 | from 1 to 113 |
| SEQ ID NO: 1612 | R0261:C10_1 | frame 2 | from 6 to 120 |
| SEQ ID NO: 1613 | R0261:C10_2 | frame 3 | from 103 to 157 |
| SEQ ID NO: 1614 | R0261:C10_3 | frame −1 | from 25 to 75 |
| SEQ ID NO: 1615 | R0261:D03_1 | frame 1 | from 44 to 179 |
| SEQ ID NO: 1616 | R0261:D03_2 | frame 2 | from 12 to 90 |
| SEQ ID NO: 1617 | R0261:D03_3 | frame 2 | from 92 to 164 |
| SEQ ID NO: 1618 | R0261:D03_4 | frame 3 | from 40 to 96 |
| SEQ ID NO: 1619 | R0261:D03_5 | frame 3 | from 98 to 186 |
| SEQ ID NO: 1620 | R0261:D03_6 | frame −1 | from 37 to 160 |
| SEQ ID NO: 1621 | R0261:D03_7 | frame −2 | from 22 to 144 |
| SEQ ID NO: 1622 | R0261:D06_1 | frame 2 | from 1 to 117 |
| SEQ ID NO: 1623 | R0261:D06_2 | frame −2 | from 1 to 117 |
| SEQ ID NO: 1624 | R0261:D06_3 | frame −3 | from 35 to 117 |
| SEQ ID NO: 1625 | R0261:E10_1 | frame 1 | from 1 to 67 |
| SEQ ID NO: 1626 | R0261:F10_1 | frame 1 | from 103 to 154 |
| SEQ ID NO: 1627 | R0261:F10_2 | frame 2 | from 5 to 106 |
| SEQ ID NO: 1628 | R0261:F10_3 | frame 3 | from 24 to 109 |
| SEQ ID NO: 1629 | R0261:F10_4 | frame −1 | from 93 to 154 |
| SEQ ID NO: 1630 | R0261:G04_1 | frame 2 | from 13 to 111 |
| SEQ ID NO: 1631 | R0261:G04_2 | frame 3 | from 91 to 147 |
| SEQ ID NO: 1632 | R0261:G04_3 | frame −1 | from 83 to 169 |
| SEQ ID NO: 1633 | R0261:G04_4 | frame −2 | from 4 to 56 |
| SEQ ID NO: 1634 | R0261:G04_5 | frame −3 | from 123 to 181 |
| SEQ ID NO: 1635 | R0262:A12_1 | frame −1 | from 35 to 132 |
| SEQ ID NO: 1636 | R0262:A03_1 | frame 2 | from 1 to 66 |
| SEQ ID NO: 1637 | R0262:A03_2 | frame −1 | from 1 to 84 |
| SEQ ID NO: 1638 | R0262:A03_3 | frame −3 | from 1 to 64 |
| SEQ ID NO: 1639 | R0262:B09_1 | frame 1 | from 1 to 59 |
| SEQ ID NO: 1640 | R0262:B09_2 | frame 2 | from 57 to 107 |
| SEQ ID NO: 1641 | R0262:B09_3 | frame 2 | from 109 to 190 |
| SEQ ID NO: 1642 | R0262:B09_4 | frame 3 | from 35 to 189 |
| SEQ ID NO: 1643 | R0262:B09_5 | frame −1 | from 1 to 55 |
| SEQ ID NO: 1644 | R0262:B09_6 | frame −1 | from 57 to 145 |
| SEQ ID NO: 1645 | R0262:B09_7 | frame −3 | from 34 to 95 |
| SEQ ID NO: 1646 | R0262:B09_8 | frame −3 | from 126 to 189 |
| SEQ ID NO: 1647 | R0262:C04_1 | frame 1 | from 18 to 75 |
| SEQ ID NO: 1648 | R0262:C04_2 | frame 2 | from 7 to 77 |
| SEQ ID NO: 1649 | R0262:C04_3 | frame −2 | from 67 to 139 |
| SEQ ID NO: 1650 | R0262:C04_4 | frame −3 | from 1 to 88 |
| SEQ ID NO: 1651 | R0262:D11_1 | frame 1 | from 22 to 90 |
| SEQ ID NO: 1652 | R0262:D11_2 | frame 2 | from 1 to 57 |
| SEQ ID NO: 1653 | R0262:D11_3 | frame 2 | from 59 to 124 |
| SEQ ID NO: 1654 | R0262:D11_4 | frame −2 | from 1 to 67 |
| SEQ ID NO: 1655 | R0262:D11_5 | frame −3 | from 26 to 124 |
| SEQ ID NO: 1656 | R0262:D12_1 | frame 2 | from 4 to 118 |
| SEQ ID NO: 1657 | R0262:D04_1 | frame 1 | from 26 to 95 |
| SEQ ID NO: 1658 | R0262:D04_2 | frame 3 | from 32 to 94 |
| SEQ ID NO: 1659 | R0262:D04_3 | frame −2 | from 16 to 65 |
| SEQ ID NO: 1660 | R0262:D04_4 | frame −3 | from 1 to 92 |
| SEQ ID NO: 1661 | R0262:D07_1 | frame 1 | from 102 to 156 |
| SEQ ID NO: 1662 | R0262:D07_2 | frame 3 | from 4 to 118 |
| SEQ ID NO: 1663 | R0262:D07_3 | frame −1 | from 20 to 70 |
| SEQ ID NO: 1664 | R0262:E02_1 | frame 1 | from 7 to 121 |
| SEQ ID NO: 1665 | R0262:E02_2 | frame 2 | from 104 to 158 |
| SEQ ID NO: 1666 | R0262:E02_3 | frame −2 | from 27 to 77 |
| SEQ ID NO: 1667 | R0262:G05_1 | frame 3 | from 50 to 111 |
| SEQ ID NO: 1668 | R0262:G05_2 | frame −1 | from 49 to 134 |
| SEQ ID NO: 1669 | R0263:B10_1 | frame 2 | from 46 to 115 |
| SEQ ID NO: 1670 | R0263:B10_2 | frame −2 | from 12 to 61 |
| SEQ ID NO: 1671 | R0263:B06_1 | frame 3 | from 1 to 115 |
| SEQ ID NO: 1672 | R0263:B06_2 | frame −1 | from 52 to 116 |
| SEQ ID NO: 1673 | R0263:B06_3 | frame −2 | from 2 to 78 |
| SEQ ID NO: 1674 | R0263:B06_4 | frame −3 | from 15 to 87 |
| SEQ ID NO: 1675 | R0263:B09_1 | frame 2 | from 1 to 199 |
| SEQ ID NO: 1676 | R0263:B09_2 | frame −1 | from 1 to 76 |
| SEQ ID NO: 1677 | R0263:B09_3 | frame −1 | from 78 to 199 |
| SEQ ID NO: 1678 | R0263:B09_4 | frame −2 from 140 to 195 | |
| SEQ ID NO: 1679 | R0263:D11_1 | frame 1 | from 12 to 98 |
| SEQ ID NO: 1680 | R0263:D11_2 | frame 3 | from 22 to 76 |
| SEQ ID NO: 1681 | R0263:D11_3 | frame −2 | from 4 to 126 |
| SEQ ID NO: 1682 | R0263:D07_1 | frame 1 | from 1 to 191 |
| SEQ ID NO: 1683 | R0263:D07_2 | frame −1 | from 1 to 51 |
| SEQ ID NO: 1684 | R0263:D07_3 | frame −1 | from 91 to 141 |
| SEQ ID NO: 1685 | R0263:D07_4 | frame −2 | from 98 to 152 |
| SEQ ID NO: 1686 | R0263:E03_1 | frame 3 | from 50 to 111 |
| SEQ ID NO: 1687 | R0263:E03_2 | frame −1 | from 119 to 204 |
| SEQ ID NO: 1688 | R0263:F08_1 | frame 3 | from 1 to 95 |
| SEQ ID NO: 1689 | R0263:G03_1 | frame 1 | from 90 to 139 |
| SEQ ID NO: 1690 | R0263:G03_2 | frame 2 | from 13 to 106 |
| SEQ ID NO: 1691 | R0263:G03_3 | frame −1 | from 3 to 55 |
| SEQ ID NO: 1692 | R0263:G03_4 | frame −2 | from 122 to 180 |
| SEQ ID NO: 1693 | R0263:G03_5 | frame −3 | from 77 to 167 |
| SEQ ID NO: 1694 | R0263:H10_1 | frame 1 | from 1 to 55 |
| SEQ ID NO: 1695 | R0263:H10_2 | frame 1 | from 99 to 152 |
| SEQ ID NO: 1696 | R0263:H10_3 | frame 3 | from 1 to 147 |
| SEQ ID NO: 1697 | R0263:H10_4 | frame −1 | from 6 to 140 |
| SEQ ID NO: 1698 | R0263:H10_5 | frame −3 | from 1 to 151 |
| SEQ ID NO: 1699 | R0263:H02_1 | frame 1 | from 4 to 63 |
| SEQ ID NO: 1700 | R0263:H02_2 | frame 1 | from 65 to 121 |
| SEQ ID NO: 1701 | R0263:H02_3 | frame 2 | from 1 to 50 |
| SEQ ID NO: 1702 | R0263:H02_4 | frame 2 | from 52 to 104 |
| SEQ ID NO: 1703 | R0263:H02_5 | frame −1 | from 13 to 77 |
| SEQ ID NO: 1704 | R0263:H02_6 | frame −1 | from 98 to 161 |
| SEQ ID NO: 1705 | R0263:H02_7 | frame −2 | from 3 to 54 |
| SEQ ID NO: 1706 | R0263:H02_8 | frame −2 | from 56 to 122 |
| SEQ ID NO: 1707 | R0264:B11_1 | frame 2 | from 6 to 120 |
| SEQ ID NO: 1708 | R0264:B11_2 | frame 3 | from 103 to 157 |
| SEQ ID NO: 1709 | R0264:B11_3 | frame −1 | from 30 to 80 |
| SEQ ID NO: 1710 | R0264:D03_1 | frame 1 | from 100 to 157 |
| SEQ ID NO: 1711 | R0264:D03_2 | frame 3 | from 16 to 91 |
| SEQ ID NO: 1712 | R0264:D03_3 | frame 3 | from 94 to 156 |
| SEQ ID NO: 1713 | R0264:D03_4 | frame −2 | from 1 to 156 |
| SEQ ID NO: 1714 | R0264:E12_1 | frame 3 | from 50 to 111 |
| SEQ ID NO: 1715 | R0264:E12_2 | frame −1 | from 78 to 163 |
| SEQ ID NO: 1716 | R0264:F11_1 | frame 1 | from 13 to 81 |
| SEQ ID NO: 1717 | R0264:F11_2 | frame −1 | from 1 to 102 |
| SEQ ID NO: 1718 | R0264:F11_3 | frame −2 | from 25 to 101 |
| SEQ ID NO: 1719 | R0264:F11_4 | frame −3 | from 42 to 101 |
| SEQ ID NO: 1720 | R0264:F09_1 | frame 1 | from 7 to 121 |
| SEQ ID NO: 1721 | R0264:F09_2 | frame 2 | from 104 to 158 |
| SEQ ID NO: 1722 | R0264:F09_3 | frame −3 | from 25 to 75 |
| SEQ ID NO: 1723 | R0264:G03_1 | frame 1 | from 1 to 62 |
| SEQ ID NO: 1724 | R0264:G03_2 | frame −1 | from 1 to 62 |
| SEQ ID NO: 1725 | R0264:G03_3 | frame −2 | from 2 to 61 |
| SEQ ID NO: 1726 | R0264:G04_1 | frame 2 | from 6 to 93 |
| SEQ ID NO: 1727 | R0264:G06_1 | frame 1 | from 50 to 106 |
| SEQ ID NO: 1728 | R0264:G09_1 | frame 2 | from 46 to 110 |
| SEQ ID NO: 1729 | R0264:G09_2 | frame −2 | from 1 to 56 |
| SEQ ID NO: 1730 | R0264:H04_1 | frame 1 | from 58 to 130 |
| SEQ ID NO: 1731 | R0264:H04_2 | frame 3 | from 78 to 129 |
| SEQ ID NO: 1732 | R0264:H04_3 | frame −1 | from 67 to 130 |
| SEQ ID NO: 1733 | R0264:H04_4 | frame −3 | from 44 to 95 |
| SEQ ID NO: 1734 | R0265:A09_1 | frame 1 | from 1 to 59 |
| SEQ ID NO: 1735 | R0265:A09_2 | frame 2 | from 54 to 107 |
| SEQ ID NO: 1736 | R0265:A09_3 | frame 3 | from 35 to 153 |
| SEQ ID NO: 1737 | R0265:A09_4 | frame −2 | from 1 to 59 |
| SEQ ID NO: 1738 | R0265:A09_5 | frame −2 | from 90 to 153 |
| SEQ ID NO: 1739 | R0265:A09_6 | frame −3 | from 20 to 108 |
| SEQ ID NO: 1740 | R0265:D10_1 | frame 1 | from 118 to 175 |
| SEQ ID NO: 1741 | R0265:D10_2 | frame 3 | from 7 to 100 |
| SEQ ID NO: 1742 | R0265:D10_3 | frame −2 | from 77 to 169 |
| SEQ ID NO: 1743 | R0265:D07_1 | frame 3 | from 50 to 111 |
| SEQ ID NO: 1744 | R0265:D07_2 | frame −1 | from 70 to 185 |
| SEQ ID NO: 1745 | R0265:E12_1 | frame 1 | from 1 to 87 |
| SEQ ID NO: 1746 | R0265:E12_2 | frame 2 | from 1 to 113 |
| SEQ ID NO: 1747 | R0265:E12_3 | frame −1 | from 1 to 79 |
| SEQ ID NO: 1748 | R0265:F12_1 | frame 1 | from 75 to 126 |
| SEQ ID NO: 1749 | R0265:F12_2 | frame 2 | from 46 to 160 |
| SEQ ID NO: 1750 | R0265:F12_3 | frame −1 | from 36 to 87 |
| SEQ ID NO: 1751 | R0265:F12_4 | frame −3 | from 78 to 133 |
| SEQ ID NO: 1752 | R0265:H04_1 | frame 2 | from 64 to 149 |
| SEQ ID NO: 1753 | R0265:H04_2 | frame −3 | from 50 to 111 |
| SEQ ID NO: 1754 | R0265:H09_1 | frame 1 | from 1 to 191 |
| SEQ ID NO: 1755 | R0265:H09_2 | frame −1 | from 1 to 51 |
| SEQ ID NO: 1756 | R0265:H09_3 | frame −1 | from 91 to 141 |
| SEQ ID NO: 1757 | R0265:H09_4 | frame −2 | from 98 to 152 |
| SEQ ID NO: 1758 | R0266:A10_1 | frame 1 | from 1 to 87 |
| SEQ ID NO: 1759 | R0266:A10_2 | frame 2 | from 1 to 113 |
| SEQ ID NO: 1760 | R0266:A10_3 | frame −1 | from 1 to 79 |
| SEQ ID NO: 1761 | R0266:A12_1 | frame 1 | from 1 to 106 |
| SEQ ID NO: 1762 | R0266:A12_2 | frame 1 | from 133 to 185 |
| SEQ ID NO: 1763 | R0266:A12_3 | frame 2 | from 89 to 143 |
| SEQ ID NO: 1764 | R0266:A12_4 | frame 2 | from 147 to 197 |
| SEQ ID NO: 1765 | R0266:A12_5 | frame −3 | from 51 to 101 |
| SEQ ID NO: 1766 | R0266:B02_1 | frame 1 | from 68 to 168 |
| SEQ ID NO: 1767 | R0266:B02_2 | frame 2 | from 44 to 167 |
| SEQ ID NO: 1768 | R0266:B02_3 | frame −1 | from 1 to 100 |
| SEQ ID NO: 1769 | R0266:C12_1 | frame 1 | from 7 to 121 |
| SEQ ID NO: 1770 | R0266:C12_2 | frame 1 | from 148 to 200 |
| SEQ ID NO: 1771 | R0266:C12_3 | frame 2 | from 104 to 158 |
| SEQ ID NO: 1772 | R0266:C12_4 | frame −3 | from 41 to 93 |
| SEQ ID NO: 1773 | R0266:E08_1 | frame 1 | from 1 to 54 |
| SEQ ID NO: 1774 | R0266:E08_2 | frame 3 | from 1 to 63 |
| SEQ ID NO: 1775 | R0266:E08_3 | frame 3 | from 128 to 195 |
| SEQ ID NO: 1776 | R0266:E08_4 | frame −1 | from 51 to 122 |
| SEQ ID NO: 1777 | R0266:E08_5 | frame −1 | from 124 to 175 |
| SEQ ID NO: 1778 | R0266:E08_6 | frame −3 | from 9 to 68 |
| SEQ ID NO: 1779 | R0266:E08_7 | frame −3 | from 83 to 134 |
| SEQ ID NO: 1780 | R0266:F03_1 | frame 1 | from 64 to 141 |
| SEQ ID NO: 1781 | R0266:F03_2 | frame 2 | from 8 to 141 |
| SEQ ID NO: 1782 | R0266:F03_3 | frame 3 | from 39 to 104 |
| SEQ ID NO: 1783 | R0266:F03_4 | frame −2 | from 1 to 141 |
| SEQ ID NO: 1784 | R0266:F06_1 | frame 1 | from 23 to 75 |
| SEQ ID NO: 1785 | R0266:F06_2 | frame 3 | from 150 to 200 |
| SEQ ID NO: 1786 | R0266:F06_3 | frame −1 | from 109 to 175 |
| SEQ ID NO: 1787 | R0266:F06_4 | frame −2 | from 143 to 200 |
| SEQ ID NO: 1788 | R0266:F07_1 | frame −3 | from 37 to 97 |
| SEQ ID NO: 1789 | R0266:F07_2 | frame −3 | from 138 to 188 |
| SEQ ID NO: 1790 | R0266:G12_1 | frame 1 | from 1 to 192 |
| SEQ ID NO: 1791 | R0266:G12_2 | frame 2 | from 94 to 168 |
| SEQ ID NO: 1792 | R0266:G12_3 | frame −2 | from 65 to 128 |
| SEQ ID NO: 1793 | R0266:G12_4 | frame −3 | from 17 to 120 |
| SEQ ID NO: 1794 | R0266:G12_5 | frame −3 | from 122 to 192 |
| SEQ ID NO: 1795 | R0266:G09_1 | frame 1 | from 7 to 121 |
| SEQ ID NO: 1796 | R0266:G09_2 | frame 2 | from 104 to 158 |
| SEQ ID NO: 1797 | R0266:G09_3 | frame −2 | from 28 to 78 |
| SEQ ID NO: 1798 | R0242:E03_1 | frame 1 | from 1 to 54 |
| SEQ ID NO: 1799 | R0242:E03_2 | frame 2 | from 38 to 93 |
| SEQ ID NO: 1800 | R0244:C04_1 | frame 1 | from 19 to 77 |
| SEQ ID NO: 1801 | R0244:C04_2 | frame 2 | from 13 to 76 |
| SEQ ID NO: 1802 | R0244:C04_3 | frame 3 | from 20 to 76 |
| SEQ ID NO: 1803 | R0244:C04_4 | frame −1 | from 15 to 65 |
| SEQ ID NO: 1804 | R0244:C06_1 | frame 2 | from 38 to 111 |
| SEQ ID NO: 1805 | R0244:C06_2 | frame −1 | from 58 to 134 |
| SEQ ID NO: 1806 | R0244:C06_3 | frame −2 | from 14 to 98 |
| SEQ ID NO: 1807 | R0245:A02_1 | frame 2 | from 12 to 61 |
| SEQ ID NO: 1808 | R0245:A02_2 | frame −3 | from 42 to 92 |
| SEQ ID NO: 1809 | R0245:D12_1 | frame 2 | from 1 to 92 |
| SEQ ID NO: 1810 | R0245:D12_2 | frame −3 | from 4 to 53 |
| SEQ ID NO: 1811 | R0246:D10_1 | frame 3 | from 30 to 95 |
| SEQ ID NO: 1812 | R0246:D10_2 | frame −1 | from 1 to 83 |
| SEQ ID NO: 1813 | R0246:D10_3 | frame −2 | from 14 to 95 |
| SEQ ID NO: 1814 | R0246:D10_4 | frame −3 | from 1 to 53 |
| SEQ ID NO: 1815 | ‘46377.1_gaiger.ABI’_1 | frame 1 | from 1 to 116 |
| SEQ ID NO: 1816 | ‘46377.1_gaiger.ABI’_2 | frame −1 | from 16 to 66 |
| SEQ ID NO: 1817 | ‘46377.1_gaiger.ABI’_3 | frame −2 | from 23 to 77 |
| SEQ ID NO: 1818 | ‘46403.1_gaiger.ABI’_1 | frame 1 | from 1 to 63 |
| SEQ ID NO: 1819 | ‘46403.1_gaiger.ABI’_2 | frame 2 | from 25 to 94 |
| SEQ ID NO: 1820 | ‘46403.1_gaiger.ABI’_3 | frame −3 | from 19 to 94 |
| SEQ ID NO: 1821 | ‘46489.1;gaiger.ABI’_1 | frame 1 | from 1 to 70 |
| SEQ ID NO: 1822 | ‘46489.1;gaiger.ABI’_2 | frame −1 | from 1 to 70 |
| SEQ ID NO: 1823 | ‘46489.1;gaiger.ABI’_3 | frame −2 | from 1 to 64 |
| SEQ ID NO: 1824 | ‘46872.1_gaiger.ABI’_1 | frame 3 | from 4 to 77 |
| SEQ ID NO: 1825 | ‘46872.1_gaiger.ABI’_2 | frame −1 | from 1 to 66 |
| SEQ ID NO: 1826 | ‘46883.1_gaiger.ABI’_1 | frame −1 | from 1 to 76 |
| SEQ ID NO: 1827 | ‘46880.1_gaiger.ABI’_1 | frame 2 | from 36 to 95 |
| SEQ ID NO: 1828 | ‘46880.1_gaiger.ABI’_2 | frame 3 | from 1 to 95 |
| SEQ ID NO: 1829 | ‘46880.1_gaiger.ABI’_3 | frame −1 | from 32 to 81 |
| SEQ ID NO: 1830 | ‘46977.1_gaiger.ABI’_1 | frame −2 | from 1 to 62 |
| SEQ ID NO: 1831 | ‘46977.1_gaiger.ABI’_2 | frame −3 | from 1 to 94 |
| SEQ ID NO: 1832 | ‘47011.1_gaiger.ABI’_1 | frame 1 | from 1 to 102 |
| SEQ ID NO: 1833 | ‘47011.1_gaiger.ABI’_2 | frame 3 | from 42 to 101 |
| SEQ ID NO: 1834 | ‘47011.1_gaiger.ABI’_3 | frame −1 | from 32 to 81 |
| SEQ ID NO: 1835 | ‘51658.1_gaiger.ABI’_1 | frame 2 | from 5 to 80 |
| SEQ ID NO: 1836 | ‘51658.1_gaiger.ABI’_2 | frame 3 | from 10 to 77 |
| SEQ ID NO: 1837 | ‘51734.1_gaiger.ABI’_1 | frame 1 | from 12 to 98 |
| SEQ ID NO: 1838 | ‘51734.1_gaiger.ABI’_2 | frame 3 | from 22 to 76 |
| SEQ ID NO: 1839 | ‘51734.1_gaiger.ABI’_3 | frame −2 | from 18 to 137 |
| SEQ ID NO: 1840 | ‘51870.1_gaiger.ABI’_1 | frame 3 | from 33 to 107 |
| SEQ ID NO: 1841 | ‘51870.1_gaiger.ABI’_2 | frame −2 | from 27 to 85 |
| SEQ ID NO: 1842 | ‘51870.1_gaiger.ABI’_3 | frame −3 | from 65 to 115 |
| SEQ ID NO: 1843 | 1404:A06_1 | frame 1 | from 29 to 102 |
| SEQ ID NO: 1844 | 1404:A06_2 | frame −3 | from 10 to 59 |
| SEQ ID NO: 1845 | 1404:B12_1 | frame 2 | from 1 to 93 |
| SEQ ID NO: 1846 | 1404:B12_2 | frame −2 | from 76 to 131 |
| SEQ ID NO: 1847 | 1404:E11_1 | frame 3 | from 1 to 64 |
| SEQ ID NO: 1848 | 1404:E11_2 | frame 3 | from 106 to 158 |
| SEQ ID NO: 1849 | 1404:E11_3 | frame −2 | from 28 to 81 |
| SEQ ID NO: 1850 | 1405:A11_1 | frame 2 | from 1 to 188 |
| SEQ ID NO: 1851 | 1405:A11_2 | frame −1 | from 20 to 69 |
| SEQ ID NO: 1852 | 1405:A11_3 | frame −2 | from 34 to 99 |
| SEQ ID NO: 1853 | 1405:A11_4 | frame −3 | from 1 to 71 |
| SEQ ID NO: 1854 | ‘52280.1_gaiger.ABI’_1 | frame 2 | from 1 to 80 |
| SEQ ID NO: 1855 | ‘52280.1_gaiger.ABI’_2 | frame 3 | from 16 to 127 |
| SEQ ID NO: 1856 | ‘52280.1_gaiger.ABI’_3 | frame −2 | from 1 to 127 |
| SEQ ID NO: 1857 | ‘52280.1_gaiger.ABI’_4 | frame −3 | from 1 to 51 |
| SEQ ID NO: 1858 | ‘52345.1_gaiger.ABI’_1 | frame 3 | from 74 to 126 |
| SEQ ID NO: 1859 | ‘52345.1_gaiger.ABI’_2 | frame −2 | from 85 to 141 |
| SEQ ID NO: 1860 | R0263:E03_1 | frame 3 | from 50 to 111 |
| SEQ ID NO: 1861 | R0263:E03_2 | frame −1 | from 119 to 204 |
| SEQ ID NO: 1862 | ‘41557.1_gaiger.ABI’_1 | frame 1 | from 16 to 73 |
| SEQ ID NO: 1863 | ‘41557.1_gaiger.ABI’_2 | frame 2 | from 1 to 109 |
| SEQ ID NO: 1864 | ‘41557.1_gaiger.ABI’_3 | frame −1 | from 11 to 110 |
| SEQ ID NO: 1865 | ‘41557.1_gaiger.ABI’_4 | frame −3 | from 1 to 103 |
| SEQ ID NO: 1866 | ‘41650.1_gaiger.ABI’_1 | frame 1 | from 22 to 109 |
| SEQ ID NO: 1867 | ‘41650.1_gaiger.ABI’_2 | frame 2 | from 1 to 157 |
| SEQ ID NO: 1868 | ‘41650.1_gaiger.ABI’_3 | frame 3 | from 1 to 156 |
| SEQ ID NO: 1869 | ‘41650.1_gaiger.ABI’_4 | frame −1 | from 25 to 99 |
| SEQ ID NO: 1870 | ‘41650.1_gaiger.ABI’_5 | frame −2 | from 47 to 157 |
| SEQ ID NO: 1871 | ‘41650.1_gaiger.ABI’_6 | frame −3 | from 53 to 156 |
| SEQ ID NO: 1872 | ‘41663.1_gaiger.ABI’_1 | frame −2 | from 64 to 116 |
| SEQ ID NO: 1873 | ‘41663.1_gaiger.ABI’_2 | frame −3 | from 1 to 67 |
| SEQ ID NO: 1874 | ‘41667.1_gaiger.ABI’_1 | frame 1 | from 1 to 56 |
| SEQ ID NO: 1875 | ‘41667.1_gaiger.ABI’_2 | frame 2 | from 1 to 56 |
| SEQ ID NO: 1876 | ‘41667.1_gaiger.ABI’_3 | frame −2 | from 1 to 56 |
| SEQ ID NO: 1877 | ‘41729.1_gaiger.ABI’_1 | frame 1 | from 64 to 114 |
| SEQ ID NO: 1878 | ‘41751.1_gaiger.ABI’_1 | frame 1 | from 27 to 82 |
| SEQ ID NO: 1879 | ‘41751.1_gaiger.ABI’_2 | frame 3 | from 1 to 50 |
| SEQ ID NO: 1880 | ‘41751.1_gaiger.ABI’_3 | frame −2 | from 1 to 70 |
| SEQ ID NO: 1881 | ‘41751.1_gaiger.ABI’_4 | frame −3 | from 1 to 53 |
| SEQ ID NO: 1882 | ‘41818.1_gaiger.ABI’_1 | frame 2 | from 1 to 69 |
| SEQ ID NO: 1883 | ‘41818.1_gaiger.ABI’_2 | frame −1 | from 30 to 93 |
| SEQ ID NO: 1884 | ‘41818.1_gaiger.ABI’_3 | frame −3 | from 1 to 92 |
| SEQ ID NO: 1885 | ‘41828.1_gaiger.ABI’_1 | frame −3 | from 1 to 77 |
| SEQ ID NO: 1886 | ‘41847.1_gaiger.ABI’_1 | frame −1 | from 1 to 97 |
| SEQ ID NO: 1887 | ‘41847.1_gaiger.ABI’_2 | frame −3 | from 1 to 56 |
| SEQ ID NO: 1888 | ‘41849.1_gaiger.ABI’_1 | frame 1 | from 1 to 75 |
| SEQ ID NO: 1889 | ‘41849.1_gaiger.ABI’_2 | frame 3 | from 4 to 77 |
| SEQ ID NO: 1890 | ‘41849.1_gaiger.ABI’_3 | frame −1 | from 12 to 77 |
| SEQ ID NO: 1891 | ‘41927.1_gaiger.ABI’_1 | frame 3 | from 20 to 74 |
| SEQ ID NO: 1892 | ‘41929.1_gaiger.ABI’_1 | frame 1 | from 1 to 52 |
| SEQ ID NO: 1893 | ‘41995.1_gaiger.ABI’_1 | frame 1 | from 1 to 115 |
| SEQ ID NO: 1894 | ‘41995.1_gaiger.ABI’_2 | frame 3 | from 60 to 109 |
| SEQ ID NO: 1895 | ‘41995.1_gaiger.ABI’_3 | frame −2 | from 1 to 114 |
| SEQ ID NO: 1896 | ‘41995.1_gaiger.ABI’_4 | frame −3 | from 35 to 108 |
| SEQ ID NO: 1897 | ‘42012.1_gaiger.ABI’_1 | frame 2 | from 1 to 60 |
| SEQ ID NO: 1898 | ‘42012.1_gaiger.ABI’_2 | frame −3 | from 1 to 60 |
| SEQ ID NO: 1899 | ‘42039.1_gaiger.ABI’_1 | frame 2 | from 70 to 127 |
| SEQ ID NO: 1900 | ‘42039.1_gaiger.ABI’_2 | frame 3 | from 1 to 146 |
| SEQ ID NO: 1901 | ‘42039.1_gaiger.ABI’_3 | frame −2 | from 39 to 100 |
| SEQ ID NO: 1902 | ‘42097.1_gaiger.ABI’_1 | frame 1 | from 24 to 132 |
| SEQ ID NO: 1903 | ‘42097.1_gaiger.ABI’_2 | frame −1 | from 52 to 132 |
| SEQ ID NO: 1904 | ‘42097.1_gaiger.ABI’_3 | frame −3 | from 34 to 92 |
| SEQ ID NO: 1905 | ‘42108.1_gaiger.ABI’_1 | frame −2 | from 4 to 77 |
| SEQ ID NO: 1906 | R0233:A06_1 | frame 1 | from 12 to 77 |
| SEQ ID NO: 1907 | R0233:A06_2 | frame 3 | from 2 to 76 |
| SEQ ID NO: 1908 | R0233:A06_3 | frame −3 | from 1 to 59 |
| SEQ ID NO: 1909 | R0233:C02_1 | frame 3 | from 26 to 90 |
| SEQ ID NO: 1910 | R0233:C02_2 | frame −2 | from 1 to 107 |
| SEQ ID NO: 1911 | R0233:C02_3 | frame −3 | from 1 to 74 |
| SEQ ID NO: 1912 | R0233:E06_1 | frame 1 | from 84 to 146 |
| SEQ ID NO: 1913 | R0233:E06_2 | frame 3 | from 1 to 181 |
| SEQ ID NO: 1914 | R0233:E06_3 | frame −2 | from 49 to 157 |
| SEQ ID NO: 1915 | R0233:F08_1 | frame 1 | from 11 to 110 |
| SEQ ID NO: 1916 | R0233:F08_2 | frame 3 | from 1 to 103 |
| SEQ ID NO: 1917 | R0233:F08_3 | frame −1 | from 16 to 73 |
| SEQ ID NO: 1918 | R0233:F08_4 | frame −2 | from 1 to 109 |
| SEQ ID NO: 1919 | ‘42328.1_gaiger.ABI’_1 | frame 2 | from 26 to 103 |
| SEQ ID NO: 1920 | ‘42328.1_gaiger.ABI’_2 | frame −2 | from 1 to 103 |
| SEQ ID NO: 1921 | ‘42588.1_gaiger.ABI’_1 | frame 1 | from 1 to 60 |
| SEQ ID NO: 1922 | ‘42588.1_gaiger.ABI’_2 | frame 2 | from 1 to 60 |
| SEQ ID NO: 1923 | ‘42588.1_gaiger.ABI’_3 | frame 3 | from 1 to 60 |
| SEQ ID NO: 1924 | ‘42588.1_gaiger.ABI’_4 | frame −1 | from 1 to 60 |
| SEQ ID NO: 1925 | ‘42588.1_gaiger.ABI’_5 | frame −2 | from 1 to 53 |
| SEQ ID NO: 1926 | ‘42703.1_gaiger.ABI’_1 | frame 2 | from 25 to 95 |
| SEQ ID NO: 1927 | ‘42703.1_gaiger.ABI’_2 | frame −2 | from 10 to 82 |
| SEQ ID NO: 1928 | R0234:B07_1 | frame 1 | from 84 to 146 |
| SEQ ID NO: 1929 | R0234:B07_2 | frame 3 | from 1 to 181 |
| SEQ ID NO: 1930 | R0234:B07_3 | frame −2 | from 49 to 157 |
| SEQ ID NO: 1931 | R0234:E06_1 | frame 3 | from 4 to 77 |
| SEQ ID NO: 1932 | R0234:E06_2 | frame −1 | from 1 to 66 |
| SEQ ID NO: 1933 | R0235:B03_1 | frame 1 | from 84 to 146 |
| SEQ ID NO: 1934 | R0235:B03_2 | frame 3 | from 1 to 169 |
| SEQ ID NO: 1935 | R0235:B03_3 | frame −1 | from 38 to 146 |
| SEQ ID NO: 1936 | R0235:E05_1 | frame 2 | from 2 to 57 |
| SEQ ID NO: 1937 | R0235:E05_2 | frame 2 | from 67 to 145 |
| SEQ ID NO: 1938 | R0235:E05_3 | frame −1 | from 74 to 187 |
| SEQ ID NO: 1939 | R0235:E05_4 | frame −2 | from 64 to 121 |
| SEQ ID NO: 1940 | R0236:A06_1 | frame 2 | from 1 to 150 |
| SEQ ID NO: 1941 | R0236:A06_2 | frame −2 | from 25 to 125 |
| SEQ ID NO: 1942 | R0236:D04_1 | frame 1 | from 1 to 87 |
| SEQ ID NO: 1943 | R0236:D04_2 | frame 2 | from 1 to 113 |
| SEQ ID NO: 1944 | R0236:D04_3 | frame −1 | from 1 to 79 |
| SEQ ID NO: 1945 | R0250:A10_1 | frame 1 | from 127 to 180 |
| SEQ ID NO: 1946 | R0250:A10_2 | frame −1 | from 1 to 55 |
| SEQ ID NO: 1947 | R0250:A10_3 | frame −2 | from 20 to 80 |
| SEQ ID NO: 1948 | R0250:A10_4 | frame −3 | from 1 to 96 |
| SEQ ID NO: 1949 | R0251:E09_1 | frame 3 | from 26 to 90 |
| SEQ ID NO: 1950 | R0251:E09_2 | frame −2 | from 1 to 107 |
| SEQ ID NO: 1951 | R0251:E09_3 | frame −3 | from 1 to 74 |
| SEQ ID NO: 1952 | R0252:F11_1 | frame 1 | from 1 to 94 |
| SEQ ID NO: 1953 | R0252:F11_2 | frame 3 | from 1 to 61 |
| SEQ ID NO: 1954 | R0252:F11_3 | frame −2 | from 12 to 69 |
| SEQ ID NO: 1955 | R0252:F11_4 | frame −3 | from 1 to 139 |
| SEQ ID NO: 1956 | R0238:B02_1 | frame 3 | from 50 to 111 |
| SEQ ID NO: 1957 | R0238:B02_2 | frame −2 | from 102 to 187 |
| SEQ ID NO: 1958 | R0239:H02_1 | frame 3 | from 1 to 97 |
| SEQ ID NO: 1959 | R0255:F12_1 | frame 3 | from 1 to 57 |
| SEQ ID NO: 1960 | R0255:F12_2 | frame −2 | from 1 to 78 |
| SEQ ID NO: 1961 | R0259:C06_1 | frame −1 | from 36 to 100 |
| SEQ ID NO: 1962 | R0259:C06_2 | frame −2 | from 124 to 187 |
| SEQ ID NO: 1963 | R0261:B10_1 | frame 1 | from 1 to 79 |
| SEQ ID NO: 1964 | R0261:B10_2 | frame −1 | from 1 to 87 |
| SEQ ID NO: 1965 | R0261:B10_3 | frame −2 | from 1 to 113 |
| SEQ ID NO: 1966 | R0261:D06_1 | frame 2 | from 1 to 117 |
| SEQ ID NO: 1967 | R0261:D06_2 | frame −2 | from 1 to 117 |
| SEQ ID NO: 1968 | R0261:D06_3 | frame −3 | from 35 to 117 |
| SEQ ID NO: 1969 | R0261:E10_1 | frame 1 | from 1 to 67 |
| SEQ ID NO: 1970 | R0261:H08_1 | frame 1 | from 29 to 102 |
| SEQ ID NO: 1971 | R0261:H08_2 | frame 2 | from 1 to 101 |
| SEQ ID NO: 1972 | R0261:H08_3 | frame 3 | from 1 to 101 |
| SEQ ID NO: 1973 | R0261:H08_4 | frame −2 | from 1 to 74 |
| SEQ ID NO: 1974 | R0261:H08_5 | frame −3 | from 38 to 101 |
| SEQ ID NO: 1975 | R0262:A12_1 | frame −1 | from 35 to 132 |
| SEQ ID NO: 1976 | R0262:A03_1 | frame 2 | from 1 to 66 |
| SEQ ID NO: 1977 | R0262:A03_2 | frame −1 | from 1 to 84 |
| SEQ ID NO: 1978 | R0262:A03_3 | frame −3 | from 1 to 64 |
| SEQ ID NO: 1979 R0262:D11_1 | frame 1 | from 22 to 90 | |
| SEQ ID NO: 1980 R0262:D11_2 | frame 2 | from 1 to 57 | |
| SEQ ID NO: 1981 | R0262:D11_3 | frame 2 | from 59 to 124 |
| SEQ ID NO: 1982 | R0262:D11_4 | frame −2 | from 1 to 67 |
| SEQ ID NO: 1983 | R0262:D11_5 | frame −3 | from 26 to 124 |
| SEQ ID NO: 1984 | R0262:E03_1 | frame 1 | from 127 to 176 |
| SEQ ID NO: 1985 | R0262:E03_2 | frame 2 | from 26 to 159 |
| SEQ ID NO: 1986 | R0262:E03_3 | frame 3 | from 1 to 67 |
| SEQ ID NO: 1987 | R0262:E03_4 | frame −1 | from 9 to 68 |
| SEQ ID NO: 1988 | R0262:E03_5 | frame −2 | from 113 to 176 |
| SEQ ID NO: 1989 | R0262:E03_6 | frame −3 | from 107 to 159 |
| SEQ ID NO: 1990 | R0262:G05_1 | frame 3 | from 50 to 111 |
| SEQ ID NO: 1991 | R0262:G05_2 | frame −1 | from 49 to 134 |
| SEQ ID NO: 1992 | R0263:B11_1 | frame 2 | from 1 to 185 |
| SEQ ID NO: 1993 | R0263:B11_2 | frame 3 | from 42 to 106 |
| SEQ ID NO: 1994 | R0263:B11_3 | frame −1 | from 74 to 185 |
| SEQ ID NO: 1995 | R0263:D11_1 | frame 1 | from 12 to 98 |
| SEQ ID NO: 1996 | R0263:D11_2 | frame 3 | from 22 to 76 |
| SEQ ID NO: 1997 | R0263:D11_3 | frame −2 | from 4 to 126 |
| SEQ ID NO: 1998 | R0263:D07_1 | frame 1 | from 1 to 191 |
| SEQ ID NO: 1999 | R0263:D07_2 | frame −1 | from 1 to 51 |
| SEQ ID NO: 2000 | R0263:D07_3 | frame −1 | from 91 to 141 |
| SEQ ID NO: 2001 | R0263:D07_4 | frame −2 | from 98 to 152 |
| SEQ ID NO: 2002 | R0263:F08_1 | frame 3 | from 1 to 95 |
| SEQ ID NO: 2003 | R0263:H02_1 | frame 1 | from 4 to 63 |
| SEQ ID NO: 2004 | R0263:H02_2 | frame 1 | from 65 to 121 |
| SEQ ID NO: 2005 | R0263:H02_3 | frame 2 | from 1 to 50 |
| SEQ ID NO: 2006 | R0263:H02_4 | frame 2 | from 52 to 104 |
| SEQ ID NO: 2007 | R0263:H02_5 | frame −1 | from 13 to 77 |
| SEQ ID NO: 2008 | R0263:H02_6 | frame −1 | from 98 to 161 |
| SEQ ID NO: 2009 | R0263:H02_7 | frame −2 | from 3 to 54 |
| SEQ ID NO: 2010 | R0263:H02_8 | frame −2 | from 56 to 122 |
| SEQ ID NO: 2011 | R0264:D03_1 | frame 1 | from 100 to 157 |
| SEQ ID NO: 2012 | R0264:D03_2 | frame 3 | from 16 to 91 |
| SEQ ID NO: 2013 | R0264:D03_3 | frame 3 | from 94 to 156 |
| SEQ ID NO: 2014 | R0264:D03_4 | frame −2 | from 1 to 156 |
| SEQ ID NO: 2015 | R0264:E12_1 | frame 3 | from 50 to 111 |
| SEQ ID NO: 2016 | R0264:E12_2 | frame −1 | from 78 to 163 |
| SEQ ID NO: 2017 | R0264:F11_1 | frame 1 | from 13 to 81 |
| SEQ ID NO: 2018 | R0264:F11_2 | frame −1 | from 1 to 102 |
| SEQ ID NO: 2019 | R0264:F11_3 | frame −2 | from 25 to 101 |
| SEQ ID NO: 2020 | R0264:F11_4 | frame −3 | from 42 to 101 |
| SEQ ID NO: 2021 | R0264:H03_1 | frame 1 | from 1 to 115 |
| SEQ ID NO: 2022 | R0264:H03_2 | frame 3 | from 60 to 109 |
| SEQ ID NO: 2023 | R0264:H03_3 | frame −2 | from 1 to 114 |
| SEQ ID NO: 2024 | R0264:H03_4 | frame −3 | from 35 to 108 |
| SEQ ID NO: 2025 | R0265:D07_1 | frame 3 | from 50 to 111 |
| SEQ ID NO: 2026 | R0265:D07_2 | frame −1 | from 70 to 185 |
| SEQ ID NO: 2027 | R0265:E12_1 | frame 1 | from 1 to 87 |
| SEQ ID NO: 2028 | R0265:E12_2 | frame 2 | from 1 to 113 |
| SEQ ID NO: 2029 | R0265:E12_3 | frame −1 | from 1 to 79 |
| SEQ ID NO: 2030 | R0265:F12_1 | frame 1 | from 75 to 126 |
| SEQ ID NO: 2031 | R0265:F12_2 | frame 2 | from 46 to 160 |
| SEQ ID NO: 2032 | R0265:F12_3 | frame −1 | from 36 to 87 |
| SEQ ID NO: 2033 | R0265:F12_4 | frame −3 | from 78 to 133 |
| SEQ ID NO: 2034 | R0265:H04_1 | frame 2 | from 64 to 149 |
| SEQ ID NO: 2035 | R0265:H04_2 | frame −3 | from 50 to 111 |
| SEQ ID NO: 2036 | R0265:H09_1 | frame 1 | from 1 to 191 |
| SEQ ID NO: 2037 | R0265:H09_2 | frame −1 | from 1 to 51 |
| SEQ ID NO: 2038 | R0265:H09_3 | frame −1 | from 91 to 141 |
| SEQ ID NO: 2039 | R0265:H09_4 | frame −2 | from 98 to 152 |
| SEQ ID NO: 2040 | R0266:A10_1 | frame 1 | from 1 to 87 |
| SEQ ID NO: 2041 | R0266:A10_2 | frame 2 | from 1 to 113 |
| SEQ ID NO: 2042 | R0266:A10_3 | frame −1 | from 1 to 79 |
| SEQ ID NO: 2043 | R0266:A12_1 | frame 1 | from 1 to 106 |
| SEQ ID NO: 2044 | R0266:A12_2 | frame 1 | from 133 to 185 |
| SEQ ID NO: 2045 | R0266:A12_3 | frame 2 | from 89 to 143 |
| SEQ ID NO: 2046 | R0266:A12_4 | frame 2 | from 147 to 197 |
| SEQ ID NO: 2047 | R0266:A12_5 | frame −3 | from 51 to 101 |
| SEQ ID NO: 2048 | R0266:F03_1 | frame 1 | from 64 to 141 |
| SEQ ID NO: 2049 | R0266:F03_2 | frame 2 | from 8 to 141 |
| SEQ ID NO: 2050 | R0266:F03_3 | frame 3 | from 39 to 104 |
| SEQ ID NO: 2051 | R0266:F03_4 | frame −2 | from 1 to 141 |
| SEQ ID NO: 2052 | R0266:F07_1 | frame −3 | from 37 to 97 |
| SEQ ID NO: 2053 | R0266:F07_2 | frame −3 | from 138 to 188 |
| SEQ ID NO: 2054 | R0266:G12_1 | frame 1 | from 1 to 192 |
| SEQ ID NO: 2055 | R0266:G12_2 | frame 2 | from 94 to 168 |
| SEQ ID NO: 2056 | R0266:G12_3 | frame −2 | from 65 to 128 |
| SEQ ID NO: 2057 | R0266:G12_4 | frame −3 | from 17 to 120 |
| SEQ ID NO: 2058 | R0266:G12_5 | frame −3 | from 122 to 192 |
| SEQ ID NO: 2059 | R0266:G09_1 | frame 1 | from 7 to 121 |
| SEQ ID NO: 2060 | R0266:G09_2 | frame 2 | from 104 to 158 |
| SEQ ID NO: 2061 | R0266:G09_3 | frame −2 | from 28 to 78 |
| SEQ ID NO: 2062 | R0244:C04_1 | frame 1 | from 19 to 77 |
| SEQ ID NO: 2063 | R0244:C04_2 | frame 2 | from 13 to 76 |
| SEQ ID NO: 2064 | R0244:C04_3 | frame 3 | from 20 to 76 |
| SEQ ID NO: 2065 | R0244:C04_4 | frame −1 | from 15 to 65 |
| SEQ ID NO: 2066 | R0245:A02_1 | frame 2 | from 12 to 61 |
| SEQ ID NO: 2067 | R0245:A02_2 | frame −3 | from 42 to 92 |
| SEQ ID NO: 2068 | R0246:D10_1 | frame 3 | from 30 to 95 |
| SEQ ID NO: 2069 | R0246:D10_2 | frame −1 | from 1 to 83 |
| SEQ ID NO: 2070 | R0246:D10_3 | frame −2 | from 14 to 95 |
| SEQ ID NO: 2071 | R0246:D10_4 | frame −3 | from 1 to 53 |
| SEQ ID NO: 2072 | ‘46403.1_gaiger.ABI’_1 | frame 1 | from 1 to 63 |
| SEQ ID NO: 2073 | ‘46403.1_gaiger.ABI’_2 | frame 2 | from 25 to 94 |
| SEQ ID NO: 2074 | ‘46403.1_gaiger.ABI’_3 | frame −3 | from 19 to 94 |
| SEQ ID NO: 2075 | ‘46458.1_gaiger.ABI’_1 | frame −3 | from 1 to 67 |
| SEQ ID NO: 2076 | ‘46489.1;gaiger.ABI’_1 | frame 1 | from 1 to 70 |
| SEQ ID NO: 2077 | ‘46489.1;gaiger.ABI’_2 | frame −1 | from 1 to 70 |
| SEQ ID NO: 2078 | ‘46489.1;gaiger.ABI’_3 | frame −2 | from 1 to 64 |
| SEQ ID NO: 2079 | ‘46802.1_gaiger.ABI’_1 | frame 1 | from 1 to 90 |
| SEQ ID NO: 2080 | ‘46802.1_gaiger.ABI’_2 | frame −1 | from 1 to 52 |
| SEQ ID NO: 2081 | ‘46872.1_gaiger.ABI’_1 | frame 3 | from 4 to 77 |
| SEQ ID NO: 2082 | ‘46872.1_gaiger.ABI’_2 | frame −1 | from 1 to 66 |
| SEQ ID NO: 2083 | ‘46880.1_gaiger.ABI’_1 | frame 2 | from 36 to 95 |
| SEQ ID NO: 2084 | ‘46880.1_gaiger.ABI’_2 | frame 3 | from 1 to 95 |
| SEQ ID NO: 2085 | ‘46880.1_gaiger.ABI’_3 | frame −1 | from 32 to 81 |
| SEQ ID NO: 2086 | ‘46977.1_gaiger.ABI’_1 | frame −2 | from 1 to 62 |
| SEQ ID NO: 2087 | ‘46977.1_gaiger.ABI’_2 | frame −3 | from 1 to 94 |
| SEQ ID NO: 2088 | ‘51658.1_gaiger.ABI’_1 | frame 2 | from 5 to 80 |
| SEQ ID NO: 2089 | ‘51658.1_gaiger.ABI’_2 | frame 3 | from 10 to 77 |
| SEQ ID NO: 2090 | ‘51734.1_gaiger.ABI’_1 | frame 1 | from 12 to 98 |
| SEQ ID NO: 2091 | ‘51734.1_gaiger.ABI’_2 | frame 3 | from 22 to 76 |
| SEQ ID NO: 2092 | ‘51734.1_gaiger.ABI’_3 | frame −2 | from 18 to 137 |
| SEQ ID NO: 2093 | 1405:C04_1 | frame 2 | from 1 to 50 |
| SEQ ID NO: 2094 | 1405:C04_2 | frame 3 | from 10 to 102 |
| SEQ ID NO: 2095 | 1405:C04_3 | frame −2 | from 76 to 140 |
| SEQ ID NO: 2096 | 1405:E11_1 | frame 1 | from 87 to 159 |
| SEQ ID NO: 2097 | 1405:E11_2 | frame 3 | from 92 to 143 |
| SEQ ID NO: 2098 | 1405:E11_3 | frame −2 | from 48 to 111 |
| SEQ ID NO: 2099 | 1405:E11_4 | frame −3 | from 1 to 55 |
| SEQ ID NO: 2100 | ‘52246.1_gaiger.ABI’_1 | frame 1 | from 12 to 98 |
| SEQ ID NO: 2101 | ‘52246.1_gaiger.ABI’_2 | frame 3 | from 22 to 76 |
| SEQ ID NO: 2102 | ‘52246.1_gaiger.ABI’_3 | frame 3 | from 78 to 127 |
| SEQ ID NO: 2103 | ‘52246.1_gaiger.ABI’_4 | frame −2 | from 5 to 127 |
| SEQ ID NO: 2104 | ‘52333.1_gaiger.ABI’_1 | frame 1 | from 1 to 69 |
| SEQ ID NO: 2105 | ‘52333.1_gaiger.ABI’_2 | frame 2 | from 1 to 66 |
| SEQ ID NO: 2106 | ‘41557.1_gaiger.ABI’_1 | frame 1 | from 16 to 73 |
| SEQ ID NO: 2107 | ‘41557.1_gaiger.ABI’_2 | frame 2 | from 1 to 109 |
| SEQ ID NO: 2108 | ‘41557.1_gaiger.ABI’_3 | frame −1 | from 11 to 110 |
| SEQ ID NO: 2109 | ‘41557.1_gaiger.ABI’_4 | frame −3 | from 1 to 103 |
| SEQ ID NO: 2110 | ‘41579.1_gaiger.ABI’_1 | frame 3 | from 43 to 97 |
| SEQ ID NO: 2111 | ‘41579.1_gaiger.ABI’_2 | frame −2 | from 1 to 97 |
| SEQ ID NO: 2112 | ‘41579.1_gaiger.ABI’_3 | frame −3 | from 43 to 97 |
| SEQ ID NO: 2113 | ‘41571.1_gaiger.ABI’_1 | frame 3 | from 1 to 89 |
| SEQ ID NO: 2114 | ‘41571.1_gaiger.ABI’_2 | frame −1 | from 1 to 89 |
| SEQ ID NO: 2115 | ‘41571.1_gaiger.ABI’_3 | frame −2 | from 27 to 85 |
| SEQ ID NO: 2116 | ‘41628.1_gaiger.ABI’_1 | frame 1 | from 51 to 121 |
| SEQ ID NO: 2117 | ‘41628.1_gaiger.ABI’_2 | frame 2 | from 1 to 97 |
| SEQ ID NO: 2118 | ‘41628.1_gaiger.ABI’_3 | frame −3 | from 47 to 98 |
| SEQ ID NO: 2119 | ‘41635.1_gaiger.ABI’_1 | frame 1 | from 1 to 70 |
| SEQ ID NO: 2120 | ‘41635.1_gaiger.ABI’_2 | frame 2 | from 31 to 127 |
| SEQ ID NO: 2121 | ‘41635.1_gaiger.ABI’_3 | frame −1 | from 56 to 127 |
| SEQ ID NO: 2122 | ‘41635.1_gaiger.ABI’_4 | frame −2 | from 76 to 126 |
| SEQ ID NO: 2123 | ‘41663.1_gaiger.ABI’_1 | frame −2 | from 64 to 116 |
| SEQ ID NO: 2124 | ‘41663.1_gaiger.ABI’_2 | frame −3 | from 1 to 67 |
| SEQ ID NO: 2125 | ‘41667.1_gaiger.ABI’_1 | frame 1 | from 1 to 56 |
| SEQ ID NO: 2126 | ‘41667.1_gaiger.ABI’_2 | frame 2 | from 1 to 56 |
| SEQ ID NO: 2127 | ‘41667.1_gaiger.ABI’_3 | frame −2 | from 1 to 56 |
| SEQ ID NO: 2128 | ‘41751.1_gaiger.ABI’_1 | frame 1 | from 27 to 82 |
| SEQ ID NO: 2129 | ‘41751.1_gaiger.ABI’_2 | frame 3 | from 1 to 50 |
| SEQ ID NO: 2130 | ‘41751.1_gaiger.ABI’_3 | frame −2 | from 1 to 70 |
| SEQ ID NO: 2131 | ‘41751.1_gaiger.ABI’_4 | frame −3 | from 1 to 53 |
| SEQ ID NO: 2132 | ‘41944.1_gaiger.ABI’_1 | frame 1 | from 1 to 56 |
| SEQ ID NO: 2133 | ‘41944.1_gaiger.ABI’_2 | frame 2 | from 1 to 177 |
| SEQ ID NO: 2134 | ‘41944.1_gaiger.ABI’_3 | frame 3 | from 37 to 92 |
| SEQ ID NO: 2135 | ‘41944.1_gaiger.ABI’_4 | frame −1 | from 47 to 116 |
| SEQ ID NO: 2136 | ‘41944.1_gaiger.ABI’_5 | frame −1 | from 125 to 177 |
| SEQ ID NO: 2137 | ‘41944.1_gaiger.ABI’_6 | frame −2 | from 32 to 177 |
| SEQ ID NO: 2138 | ‘41944.1_gaiger.ABI’_7 | frame −3 | from 120 to 177 |
| SEQ ID NO: 2139 | ‘41986.1_gaiger.ABI’_1 | frame 3 | from 1 to 110 |
| SEQ ID NO: 2140 | ‘41986.1_gaiger.ABI’_2 | frame −1 | from 1 to 110 |
| SEQ ID NO: 2141 | ‘41986.1_gaiger.ABI’_3 | frame −3 | from 22 to 91 |
| SEQ ID NO: 2142 | ‘42101.1_gaiger.ABI’_1 | frame 3 | from 53 to 123 |
| SEQ ID NO: 2143 | ‘42101.1_gaiger.ABI’_2 | frame −2 | from 1 to 124 |
| SEQ ID NO: 2144 | R0232:E07_1 | frame 2 | from 1 to 51 |
| SEQ ID NO: 2145 | R0232:E07_2 | frame −1 | from 1 to 51 |
| SEQ ID NO: 2146 | R0233:A06_1 | frame 1 | from 12 to 77 |
| SEQ ID NO: 2147 | R0233:A06_2 | frame 3 | from 2 to 76 |
| SEQ ID NO: 2148 | R0233:A06_3 | frame −3 | from 1 to 59 |
| SEQ ID NO: 2149 | ‘42324.1_gaiger.ABI’_1 | frame 1 | from 1 to 94 |
| SEQ ID NO: 2150 | ‘42324.1_gaiger.ABI’_2 | frame 2 | from 1 to 57 |
| SEQ ID NO: 2151 | ‘42324.1_gaiger.ABI’_3 | frame 3 | from 38 to 130 |
| SEQ ID NO: 2152 | ‘42324.1_gaiger.ABI’_4 | frame −1 | from 10 to 130 |
| SEQ ID NO: 2153 | ‘42324.1_gaiger.ABI’_5 | frame −2 | from 1 to 54 |
| SEQ ID NO: 2154 | ‘42324.1_gaiger.ABI’_6 | frame −2 | from 72 to 130 |
| SEQ ID NO: 2155 | ‘42324.1_gaiger.ABI’_7 | frame −3 | from 1 to 67 |
| SEQ ID NO: 2156 | ‘42324.1_gaiger.ABI’_8 | frame −3 | from 76 to 130 |
| SEQ ID NO: 2157 | ‘42438.1_gaiger.ABI’_1 | frame 1 | from 1 to 123 |
| SEQ ID NO: 2158 | ‘42438.1_gaiger.ABI’_2 | frame −3 | from 53 to 123 |
| SEQ ID NO: 2159 | ‘42625.1_gaiger.ABI’_1 | frame 1 | from 1 to 62 |
| SEQ ID NO: 2160 | ‘42702.1_gaiger.ABI’_1 | frame 1 | from 1 to 53 |
| SEQ ID NO: 2161 | ‘42702.1_gaiger.ABI’_2 | frame 2 | from 19 to 101 |
| SEQ ID NO: 2162 | ‘42702.1_gaiger.ABI’_3 | frame 3 | from 35 to 149 |
| SEQ ID NO: 2163 | ‘42702.1_gaiger.ABI’_4 | frame −1 | from 4 to 168 |
| SEQ ID NO: 2164 | ‘42702.1_gaiger.ABI’_5 | frame −2 | from 28 to 118 |
| SEQ ID NO: 2165 | ‘42702.1_gaiger.ABI’_6 | frame −2 | from 120 to 185 |
| SEQ ID NO: 2166 | ‘42702.1_gaiger.ABI’_7 | frame −3 | from 104 to 185 |
| SEQ ID NO: 2167 | ‘42709.1_gaiger.ABI’_1 | frame 2 | from 1 to 118 |
| SEQ ID NO: 2168 | ‘42709.1_gaiger.ABI’_2 | frame −3 | from 53 to 118 |
| SEQ ID NO: 2169 | R0234:E07_1 | frame 2 | from 1 to 86 |
| SEQ ID NO: 2170 | R0234:E07_2 | frame 3 | from 27 to 86 |
| SEQ ID NO: 2171 | R0234:E07_3 | frame −1 | from 1 to 51 |
| SEQ ID NO: 2172 | R0234:G11_1 | frame 3 | from 1 to 121 |
| SEQ ID NO: 2173 | R0234:G11_2 | frame −2 | from 51 to 121 |
| SEQ ID NO: 2174 | R0236:A09_1 | frame 3 | from 1 to 122 |
| SEQ ID NO: 2175 | R0236:A09_2 | frame −1 | from 54 to 122 |
| SEQ ID NO: 2176 | R0250:A05_1 | frame 1 | from 22 to 85 |
| SEQ ID NO: 2177 | R0250:A05_2 | frame 3 | from 1 to 179 |
| SEQ ID NO: 2178 | R0250:A05_3 | frame −1 | from 96 to 159 |
| SEQ ID NO: 2179 | R0250:A05_4 | frame −2 | from 34 to 85 |
| SEQ ID NO: 2180 | R0250:A05_5 | frame −2 | from 87 to 150 |
| SEQ ID NO: 2181 | R0251:A07_1 | frame 1 | from 43 to 176 |
| SEQ ID NO: 2182 | R0251:A07_2 | frame 2 | from 48 to 176 |
| SEQ ID NO: 2183 | R0251:A07_3 | frame −2 | from 1 to 129 |
| SEQ ID NO: 2184 | R0251:A07_4 | frame −3 | from 80 to 134 |
| SEQ ID NO: 2185 | R0251:D01_1 | frame 2 | from 1 to 124 |
| SEQ ID NO: 2186 | R0251:D01_2 | frame −3 | from 53 to 123 |
| SEQ ID NO: 2187 | R0252:A08_1 | frame 1 | from 1 to 64 |
| SEQ ID NO: 2188 | R0252:A08_2 | frame 2 | from 12 to 64 |
| SEQ ID NO: 2189 | R0252:A08_3 | frame −1 | from 1 to 51 |
| SEQ ID NO: 2190 | R0252:A08_4 | frame −2 | from 1 to 64 |
| SEQ ID NO: 2191 | R0252:F11_1 | frame 1 | from 1 to 94 |
| SEQ ID NO: 2192 | R0252:F11_2 | frame 3 | from 1 to 61 |
| SEQ ID NO: 2193 | R0252:F11_3 | frame −2 | from 12 to 69 |
| SEQ ID NO: 2194 | R0252:F11_4 | frame −3 | from 1 to 139 |
| SEQ ID NO: 2195 | R0252:H01_1 | frame 2 | from 1 to 123 |
| SEQ ID NO: 2196 | R0252:H01_2 | frame −3 | from 53 to 123 |
| SEQ ID NO: 2197 | R0253:G05_1 | frame 3 | from 53 to 123 |
| SEQ ID NO: 2198 | R0253:G05_2 | frame −2 | from 1 to 124 |
| SEQ ID NO: 2199 | R0254:F07_1 | frame 1 | from 69 to 153 |
| SEQ ID NO: 2200 | R0254:F07_2 | frame −1 | from 87 to 142 |
| SEQ ID NO: 2201 | R0254:F07_3 | frame −2 | from 47 to 116 |
| SEQ ID NO: 2202 | R0254:F07_4 | frame −3 | from 1 to 82 |
| SEQ ID NO: 2203 | R0254:F07_5 | frame −3 | from 99 to 154 |
| SEQ ID NO: 2204 | R0255:F12_1 | frame 3 | from 1 to 57 |
| SEQ ID NO: 2205 | R0255:F12_2 | frame −2 | from 1 to 78 |
| SEQ ID NO: 2206 | R0259:C04_1 | frame 1 | from 16 to 78 |
| SEQ ID NO: 2207 | R0259:C04_2 | frame −2 | from 53 to 139 |
| SEQ ID NO: 2208 | R0261:A09_1 | frame 1 | from 1 to 174 |
| SEQ ID NO: 2209 | R0261:A09_2 | frame 2 | from 34 to 89 |
| SEQ ID NO: 2210 | R0261:A09_3 | frame 3 | from 1 to 52 |
| SEQ ID NO: 2211 | R0261:A09_4 | frame −1 | from 121 to 174 |
| SEQ ID NO: 2212 | R0261:A09_5 | frame −2 | from 47 to 116 |
| SEQ ID NO: 2213 | R0261:A09_6 | frame −2 | from 125 to 174 |
| SEQ ID NO: 2214 | R0261:A09_7 | frame −3 | from 32 to 174 |
| SEQ ID NO: 2215 | R0261:C10_1 | frame 2 | from 6 to 120 |
| SEQ ID NO: 2216 | R0261:C10_2 | frame 3 | from 103 to 157 |
| SEQ ID NO: 2217 | R0261:C10_3 | frame −1 | from 25 to 75 |
| SEQ ID NO: 2218 | R0261:D06_1 | frame 2 | from 1 to 117 |
| SEQ ID NO: 2219 | R0261:D06_2 | frame −2 | from 1 to 117 |
| SEQ ID NO: 2220 | R0261:D06_3 | frame −3 | from 35 to 117 |
| SEQ ID NO: 2221 | R0262:D04_1 | frame 1 | from 26 to 95 |
| SEQ ID NO: 2222 | R0262:D04_2 | frame 3 | from 32 to 94 |
| SEQ ID NO: 2223 | R0262:D04_3 | frame −2 | from 16 to 65 |
| SEQ ID NO: 2224 | R0262:D04_4 | frame −3 | from 1 to 92 |
| SEQ ID NO: 2225 | R0262:E03_1 | frame 1 | from 127 to 176 |
| SEQ ID NO: 2226 | R0262:E03_2 | frame 2 | from 26 to 159 |
| SEQ ID NO: 2227 | R0262:E03_3 | frame 3 | from 1 to 67 |
| SEQ ID NO: 2228 | R0262:E03_4 | frame −1 | from 9 to 68 |
| SEQ ID NO: 2229 | R0262:E03_5 | frame −2 | from 113 to 176 |
| SEQ ID NO: 2230 | R0262:E03_6 | frame −3 | from 107 to 159 |
| SEQ ID NO: 2231 | R0263:B11_1 | frame 2 | from 1 to 185 |
| SEQ ID NO: 2232 | R0263:B11_2 | frame 3 | from 42 to 106 |
| SEQ ID NO: 2233 | R0263:B11_3 | frame −1 | from 74 to 185 |
| SEQ ID NO: 2234 | R0263:B09_1 | frame 2 | from 1 to 199 |
| SEQ ID NO: 2235 | R0263:B09_2 | frame −1 | from 1 to 76 |
| SEQ ID NO: 2236 | R0263:B09_3 | frame −1 | from 78 to 199 |
| SEQ ID NO: 2237 | R0263:B09_4 | frame −2 | from 140 to 195 |
| SEQ ID NO: 2238 | R0263:C08_1 | frame 1 | from 1 to 50 |
| SEQ ID NO: 2239 | R0263:C08_2 | frame 1 | from 52 to 101 |
| SEQ ID NO: 2240 | R0263:C08_3 | frame 1 | from 161 to 215 |
| SEQ ID NO: 2241 | R0263:C08_4 | frame 2 | from 55 to 129 |
| SEQ ID NO: 2242 | R0263:C08_5 | frame 3 | from 45 to 147 |
| SEQ ID NO: 2243 | R0263:C08_6 | frame −1 | from 113 to 188 |
| SEQ ID NO: 2244 | R0263:C08_7 | frame −2 | from 1 to 194 |
| SEQ ID NO: 2245 | R0263:D11_1 | frame 1 | from 12 to 98 |
| SEQ ID NO: 2246 | R0263:D11_2 | frame 3 | from 22 to 76 |
| SEQ ID NO: 2247 | R0263:D11_3 | frame −2 | from 1 to 119 |
| SEQ ID NO: 2248 | R0263:H10_1 | frame 1 | from 1 to 55 |
| SEQ ID NO: 2249 | R0263:H10_2 | frame 1 | from 99 to 152 |
| SEQ ID NO: 2250 | R0263:H10_3 | frame 3 | from 1 to 147 |
| SEQ ID NO: 2251 | R0263:H10_4 | frame −1 | from 6 to 140 |
| SEQ ID NO: 2252 | R0263:H10_5 | frame −3 | from 1 to 151 |
| SEQ ID NO: 2253 | R0264:A03_1 | frame 2 | from 18 to 77 |
| SEQ ID NO: 2254 | R0264:A03_2 | frame 3 | from 59 to 128 |
| SEQ ID NO: 2255 | R0264:A03_3 | frame −2 | from 53 to 129 |
| SEQ ID NO: 2256 | R0264:B11_1 | frame 2 | from 6 to 120 |
| SEQ ID NO: 2257 | R0264:B11_2 | frame 3 | from 103 to 157 |
| SEQ ID NO: 2258 | R0264:B11_3 | frame −1 | from 30 to 80 |
| SEQ ID NO: 2259 | R0264:F11_1 | frame 1 | from 13 to 81 |
| SEQ ID NO: 2260 R0264:F11_2 | frame −1 | from 1 to 102 | |
| SEQ ID NO: 2261 | R0264:F11_3 | frame −2 | from 25 to 101 |
| SEQ ID NO: 2262 R0264:F11_4 | frame −3 | from 42 to 101 | |
| SEQ ID NO: 2263 | R0264:F05_1 | frame 2 | from 18 to 77 |
| SEQ ID NO: 2264 | R0264:F05_2 | frame 3 | from 59 to 113 |
| SEQ ID NO: 2265 | R0264:F05_3 | frame −1 | from 38 to 114 |
| SEQ ID NO: 2266 | R0264:F09_1 | frame 1 | from 7 to 121 |
| SEQ ID NO: 2267 | R0264:F09_2 | frame 2 | from 104 to 158 |
| SEQ ID NO: 2268 | R0264:F09_3 | frame −3 | from 25 to 75 |
| SEQ ID NO: 2269 | R0266:B02_1 | frame 1 | from 68 to 168 |
| SEQ ID NO: 2270 | R0266:B02_2 | frame 2 | from 44 to 167 |
| SEQ ID NO: 2271 | R0266:B02_3 | frame −1 | from 1 to 100 |
| SEQ ID NO: 2272 | R0266:B03_1 | frame 2 | from 47 to 100 |
| SEQ ID NO: 2273 | R0266:B03_2 | frame 3 | from 13 to 84 |
| SEQ ID NO: 2274 | R0266:B03_3 | frame −1 | from 47 to 101 |
| SEQ ID NO: 2275 | R0266:B03_4 | frame −2 | from 27 to 100 |
| SEQ ID NO: 2276 | R0266:B04_1 | frame 1 | from 53 to 121 |
| SEQ ID NO: 2277 | R0266:B04_2 | frame 3 | from 1 to 120 |
| SEQ ID NO: 2278 | R0266:B04_3 | frame −2 | from 1 to 100 |
| SEQ ID NO: 2279 | R0266:B04_4 | frame −3 | from 7 to 106 |
| SEQ ID NO: 2280 | R0266:B06_1 | frame 1 | from 28 to 102 |
| SEQ ID NO: 2281 | R0266:B06_2 | frame 1 | from 104 to 154 |
| SEQ ID NO: 2282 | R0266:B06_3 | frame 2 | from 1 to 57 |
| SEQ ID NO: 2283 | R0266:B06_4 | frame 3 | from 1 to 68 |
| SEQ ID NO: 2284 | R0266:B06_5 | frame −1 | from 7 to 72 |
| SEQ ID NO: 2285 | R0266:B06_6 | frame −1 | from 111 to 169 |
| SEQ ID NO: 2286 | R0266:B06_7 | frame −2 | from 1 to 50 |
| SEQ ID NO: 2287 | R0266:B06_8 | frame −3 | from 50 to 136 |
| SEQ ID NO: 2288 | R0266:D05_1 | frame 1 | from 71 to 158 |
| SEQ ID NO: 2289 | R0266:D05_2 | frame −1 | from 96 to 148 |
| SEQ ID NO: 2290 | R0266:D05_3 | frame −2 | from 56 to 106 |
| SEQ ID NO: 2291 | R0266:D05_4 | frame −3 | from 63 to 143 |
| SEQ ID NO: 2292 | R0266:E01_1 | frame 3 | from 1 to 125 |
| SEQ ID NO: 2293 | R0266:E01_2 | frame −1 | from 75 to 133 |
| SEQ ID NO: 2294 | R0266:E01_3 | frame −2 | from 34 to 133 |
| SEQ ID NO: 2295 | R0266:E03_1 | frame 3 | from 81 to 130 |
| SEQ ID NO: 2296 | R0266:E03_2 | frame −1 | from 1 to 131 |
| SEQ ID NO: 2297 | R0266:E03_3 | frame −3 | from 1 to 53 |
| SEQ ID NO: 2298 | R0266:F03_1 | frame 1 | from 64 to 141 |
| SEQ ID NO: 2299 | R0266:F03_2 | frame 2 | from 8 to 141 |
| SEQ ID NO: 2300 | R0266:F03_3 | frame 3 | from 39 to 104 |
| SEQ ID NO: 2301 | R0266:F03_4 | frame −2 | from 1 to 141 |
| SEQ ID NO: 2302 | R0266:F09_1 | frame 1 | from 1 to 50 |
| SEQ ID NO: 2303 | R0266:F09_2 | frame 1 | from 52 to 101 |
| SEQ ID NO: 2304 | R0266:F09_3 | frame 2 | from 55 to 129 |
| SEQ ID NO: 2305 | R0266:F09_4 | frame 2 | from 139 to 198 |
| SEQ ID NO: 2306 | R0266:F09_5 | frame 3 | from 45 to 147 |
| SEQ ID NO: 2307 | R0266:F09_6 | frame −1 | from 1 to 177 |
| SEQ ID NO: 2308 | R0266:F09_7 | frame −2 | from 29 to 97 |
| SEQ ID NO: 2309 | R0266:F09_8 | frame −3 | from 95 to 170 |
| SEQ ID NO: 2310 | R0245:A02_1 | frame 2 | from 12 to 61 |
| SEQ ID NO: 2311 | R0245:A02_2 | frame −3 | from 42 to 92 |
| SEQ ID NO: 2312 | ‘46403.1_gaiger.ABI’_1 | frame 1 | from 1 to 63 |
| SEQ ID NO: 2313 | ‘46403.1_gaiger.ABI’_2 | frame 2 | from 25 to 94 |
| SEQ ID NO: 2314 | ‘46403.1_gaiger.ABI’_3 | frame −3 | from 19 to 94 |
| SEQ ID NO: 2315 | ‘46458.1_gaiger.ABI’_1 | frame −3 | from 1 to 67 |
| SEQ ID NO: 2316 | ‘46977.1_gaiger.ABI’_1 | frame −2 | from 1 to 62 |
| SEQ ID NO: 2317 | ‘46977.1_gaiger.ABI’_2 | frame −3 | from 1 to 94 |
| SEQ ID NO: 2318 | ‘51658.1_gaiger.ABI’_1 | frame 2 | from 5 to 80 |
| SEQ ID NO: 2319 | ‘51658.1_gaiger.ABI’_2 | frame 3 | from 10 to 77 |
| SEQ ID NO: 2320 | ‘51788.1_gaiger.ABI’_1 | frame 1 | from 1 to 59 |
| SEQ ID NO: 2321 | ‘51788.1_gaiger.ABI’_2 | frame −2 | from 1 to 68 |
| SEQ ID NO: 2322 | ‘51850.1_gaiger.ABI’_1 | frame 2 | from 1 to 58 |
| SEQ ID NO: 2323 | ‘51850.1_gaiger.ABI’_2 | frame 3 | from 47 to 106 |
| SEQ ID NO: 2324 | ‘51850.1_gaiger.ABI’_3 | frame −3 | from 1 to 67 |
| SEQ ID NO: 2325 | ‘51892.1_gaiger.ABI’_1 | frame 1 | from 1 to 158 |
| SEQ ID NO: 2326 | ‘51892.1_gaiger.ABI’_2 | frame 2 | from 2 to 69 |
| SEQ ID NO: 2327 | ‘51892.1_gaiger.ABI’_3 | frame −1 | from 76 to 139 |
| SEQ ID NO: 2328 | ‘51892.1_gaiger.ABI’_4 | frame −2 | from 35 to 137 |
| SEQ ID NO: 2329 | ‘51900.1_gaiger.ABI’_1 | frame 2 | from 1 to 123 |
| SEQ ID NO: 2330 | ‘51900.1_gaiger.ABI’_2 | frame 3 | from 3 to 70 |
| SEQ ID NO: 2331 | ‘51900.1_gaiger.ABI’_3 | frame −2 | from 78 to 141 |
| SEQ ID NO: 2332 | ‘51900.1_gaiger.ABI’_4 | frame −3 | from 36 to 139 |
| SEQ ID NO: 2333 | ‘51960.1_gaiger.ABI’_1 | frame 3 | from 61 to 133 |
| SEQ ID NO: 2334 | 1405:A09_1 | frame 1 | from 66 to 131 |
| SEQ ID NO: 2335 | 1405:A09_2 | frame −1 | from 19 to 77 |
| SEQ ID NO: 2336 | 1405:A09_3 | frame −3 | from 1 to 68 |
| SEQ ID NO: 2337 | 1405:A09_4 | frame −3 | from 117 to 174 |
| SEQ ID NO: 2338 | 1405:D12_1 | frame 1 | from 4 to 71 |
| SEQ ID NO: 2339 | 1405:D12_2 | frame 3 | from 1 to 143 |
| SEQ ID NO: 2340 | 1405:D12_3 | frame −1 | from 52 to 115 |
| SEQ ID NO: 2341 | 1405:D12_4 | frame −2 | from 11 to 113 |
| SEQ ID NO: 2342 | 1405:D09_1 | frame 1 | from 9 to 170 |
| SEQ ID NO: 2343 | 1405:D09_2 | frame 2 | from 1 to 55 |
| SEQ ID NO: 2344 | 1405:D09_3 | frame 2 | from 104 to 169 |
| SEQ ID NO: 2345 | 1405:D09_4 | frame −1 | from 1 to 98 |
| SEQ ID NO: 2346 | 1405:D09_5 | frame −2 | from 5 to 104 |
| SEQ ID NO: 2347 | 1405:E11_1 | frame 1 | from 87 to 159 |
| SEQ ID NO: 2348 | 1405:E11_2 | frame 3 | from 92 to 143 |
| SEQ ID NO: 2349 | 1405:E11_3 | frame −2 | from 48 to 111 |
| SEQ ID NO: 2350 | 1405:E11_4 | frame −3 | from 1 to 55 |
| SEQ ID NO: 2351 | ‘52246.1_gaiger.ABI’_1 | frame 1 | from 12 to 98 |
| SEQ ID NO: 2352 | ‘52246.1_gaiger.ABI’_2 | frame 3 | from 22 to 76 |
| SEQ ID NO: 2353 | ‘52246.1_gaiger.ABI’_3 | frame 3 | from 78 to 127 |
| SEQ ID NO: 2354 | ‘52246.1_gaiger.ABI’_4 | frame −2 | from 5 to 127 |
| SEQ ID NO: 2355 | ‘52333.1_gaiger.ABI’_1 | frame 1 | from 1 to 69 |
| SEQ ID NO: 2356 | ‘52333.1_gaiger.ABI’_2 | frame 2 | from 1 to 66 |
| SEQ ID NO: 2357 | 1408:A09_1 | frame 2 | from 1 to 156 |
| SEQ ID NO: 2358 | 1408:A09_2 | frame 3 | from 1 to 67 |
| SEQ ID NO: 2359 | 1408:A09_3 | frame −2 | from 94 to 157 |
| SEQ ID NO: 2360 | 1408:A09_4 | frame −3 | from 53 to 155 |
| SEQ ID NO: 2361 | 1408:B02_1 | frame 2 | from 8 to 187 |
| SEQ ID NO: 2362 | 1408:B02_2 | frame −1 | from 9 to 80 |
| SEQ ID NO: 2363 | 1408:B02_3 | frame −1 | from 82 to 175 |
| SEQ ID NO: 2364 | 1408:B02_4 | frame −3 | from 29 to 78 |
| SEQ ID NO: 2365 | 1408:B02_5 | frame −3 | from 118 to 187 |
| SEQ ID NO: 2366 | 1408:C12_1 | frame 2 | from 122 to 175 |
| SEQ ID NO: 2367 | 1408:C12_2 | frame 3 | from 1 to 187 |
| SEQ ID NO: 2368 | 1408:C12_3 | frame −2 | from 1 to 71 |
| SEQ ID NO: 2369 | 1408:C12_4 | frame −3 | from 1 to 84 |
| SEQ ID NO: 2370 | 1408:C12_5 | frame −3 | from 86 to 137 |
| SEQ ID NO: 2371 | 1408:D06_1 | frame 1 | from 127 to 180 |
| SEQ ID NO: 2372 | 1408:D06_2 | frame 2 | from 1 to 161 |
| SEQ ID NO: 2373 | 1408:D06_3 | frame 3 | from 35 to 166 |
| SEQ ID NO: 2374 | 1408:D06_4 | frame −1 | from 61 to 183 |
| SEQ ID NO: 2375 | 1408:D06_5 | frame −3 | from 7 to 63 |
| SEQ ID NO: 2376 | ‘41663.1_gaiger.ABI’_1 | frame −2 | from 64 to 116 |
| SEQ ID NO: 2377 | ‘41663.1_gaiger.ABI’_2 | frame −3 | from 1 to 67 |
| SEQ ID NO: 2378 | ‘41729.1_gaiger.ABI’_1 | frame 1 | from 64 to 114 |
| SEQ ID NO: 2379 | ‘41888.1_gaiger.ABI’_1 | frame 3 | from 22 to 81 |
| SEQ ID NO: 2380 | ‘41925.1_gaiger.ABI’_1 | frame 1 | from 9 to 59 |
| SEQ ID NO: 2381 | ‘41925.1_gaiger.ABI’_2 | frame 2 | from 1 to 59 |
| SEQ ID NO: 2382 | ‘41925.1_gaiger.ABI’_3 | frame −2 | from 1 to 59 |
| SEQ ID NO: 2383 | ‘41925.1_gaiger.ABI’_4 | frame −3 | from 1 to 58 |
| SEQ ID NO: 2384 | ‘41639.1_gaiger.ABI’_1 | frame 1 | from 1 to 135 |
| SEQ ID NO: 2385 | ‘41639.1_gaiger.ABI’_2 | frame 2 | from 86 to 135 |
| SEQ ID NO: 2386 | ‘41639.1_gaiger.ABI’_3 | frame 3 | from 1 to 134 |
| SEQ ID NO: 2387 | ‘41639.1_gaiger.ABI’_4 | frame −1 | from 1 to 84 |
| SEQ ID NO: 2388 | ‘41639.1_gaiger.ABI’_5 | frame −1 | from 86 to 135 |
| SEQ ID NO: 2389 | ‘41639.1_gaiger.ABI’_6 | frame −3 | from 39 to 124 |
| SEQ ID NO: 2390 | ‘41853.1_gaiger.ABI’_1 | frame 1 | from 13 to 90 |
| SEQ ID NO: 2391 | ‘41853.1_gaiger.ABI’_2 | frame 2 | from 28 to 78 |
| SEQ ID NO: 2392 | ‘41853.1_gaiger.ABI’_3 | frame 3 | from 1 to 50 |
| SEQ ID NO: 2393 | ‘41853.1_gaiger.ABI’_4 | frame 3 | from 66 to 147 |
| SEQ ID NO: 2394 | ‘41853.1_gaiger.ABI’_5 | frame −3 | from 35 to 112 |
| SEQ ID NO: 2395 | ‘41924.1_gaiger.ABI’_1 | frame 2 | from 1 to 64 |
| SEQ ID NO: 2396 | ‘41924.1_gaiger.ABI’_2 | frame −2 | from 27 to 83 |
| SEQ ID NO: 2397 | ‘41638.1_gaiger.ABI’_1 | frame 1 | from 8 to 81 |
| SEQ ID NO: 2398 | ‘41638.1_gaiger.ABI’_2 | frame 3 | from 6 to 62 |
| SEQ ID NO: 2399 | ‘41638.1_gaiger.ABI’_3 | frame −2 | from 1 to 58 |
| SEQ ID NO: 2400 | ‘41629.1_gaiger.ABI’_1 | frame 1 | from 1 to 59 |
| SEQ ID NO: 2401 | ‘41629.1_gaiger.ABI’_2 | frame 2 | from 38 to 87 |
| SEQ ID NO: 2402 | ‘41629.1_gaiger.ABI’_3 | frame −3 | from 42 to 91 |
| SEQ ID NO: 2403 | ‘41678.1_gaiger.ABI’_1 | frame −2 | from 1 to 60 |
| SEQ ID NO: 2404 | ‘41717.1_gaiger.ABI’_1 | frame 1 | from 55 to 129 |
| SEQ ID NO: 2405 | ‘41717.1_gaiger.ABI’_2 | frame 2 | from 1 to 63 |
| SEQ ID NO: 2406 | ‘41717.1_gaiger.ABI’_3 | frame −3 | from 1 to 68 |
| SEQ ID NO: 2407 | ‘41987.1_gaiger.ABI’_1 | frame 1 | from 48 to 116 |
| SEQ ID NO: 2408 | ‘41987.1_gaiger.ABI’_2 | frame 2 | from 1 to 50 |
| SEQ ID NO: 2409 | ‘41987.1_gaiger.ABI’_3 | frame 2 | from 96 to 154 |
| SEQ ID NO: 2410 | ‘41987.1_gaiger.ABI’_4 | frame 3 | from 53 to 120 |
| SEQ ID NO: 2411 | ‘41987.1_gaiger.ABI’_5 | frame 3 | from 122 to 175 |
| SEQ ID NO: 2412 | ‘41987.1_gaiger.ABI’_6 | frame −1 | from 37 to 136 |
| SEQ ID NO: 2413 | ‘41987.1_gaiger.ABI’_7 | frame −2 | from 1 to 72 |
| SEQ ID NO: 2414 | R0233:F02_1 | frame 2 | from 119 to 190 |
| SEQ ID NO: 2415 | R0233:F02_2 | frame 3 | from 1 to 76 |
| SEQ ID NO: 2416 | R0233:F02_3 | frame −1 | from 31 to 82 |
| SEQ ID NO: 2417 | R0233:F02_4 | frame −1 | from 112 to 190 |
| SEQ ID NO: 2418 | R0232:A08_1 | frame −2 | from 4 to 64 |
| SEQ ID NO: 2419 | R0233:B04_1 | frame 2 | from 76 to 136 |
| SEQ ID NO: 2420 | R0233:B04_2 | frame −3 | from 1 to 103 |
| SEQ ID NO: 2421 | ‘42041.1_gaiger.ABI’_1 | frame −3 | from 1 to 63 |
| SEQ ID NO: 2422 | ‘42407.1_gaiger.ABI’_1 | frame 1 | from 32 to 83 |
| SEQ ID NO: 2423 | ‘42407.1_gaiger.ABI’_2 | frame 3 | from 1 to 90 |
| SEQ ID NO: 2424 | ‘42407.1_gaiger.ABI’_3 | frame −1 | from 32 to 87 |
| SEQ ID NO: 2425 | ‘42407.1_gaiger.ABI’_4 | frame −2 | from 10 to 100 |
| SEQ ID NO: 2426 | ‘42483.1;gaiger.ABI’_1 | frame 3 | from 4 to 81 |
| SEQ ID NO: 2427 | ‘42483.1;gaiger.ABI’_2 | frame −2 | from 12 to 75 |
| SEQ ID NO: 2428 | ‘42483.1;gaiger.ABI’_3 | frame −3 | from 1 to 51 |
| SEQ ID NO: 2429 | ‘42350.1_gaiger.ABI’_1 | frame −1 | from 12 to 90 |
| SEQ ID NO: 2430 | ‘42530.1;gaiger.ABI’_1 | frame 1 | from 7 to 73 |
| SEQ ID NO: 2431 | ‘42530.1;gaiger.ABI’_2 | frame −3 | from 3 to 72 |
| SEQ ID NO: 2432 | ‘42523.1;gaiger.ABI’_1 | frame 2 | from 19 to 74 |
| SEQ ID NO: 2433 | ‘42523.1;gaiger.ABI’_2 | frame 3 | from 2 to 51 |
| SEQ ID NO: 2434 | ‘42523.1;gaiger.ABI’_3 | frame −2 | from 1 to 58 |
| SEQ ID NO: 2435 | R0235:D07_1 | frame 3 | from 33 to 110 |
| SEQ ID NO: 2436 | R0235:D07_2 | frame −2 | from 64 to 113 |
| SEQ ID NO: 2437 | R0235:D07_3 | frame −3 | from 34 to 150 |
| SEQ ID NO: 2438 | R0235:D12_1 | frame −2 | from 18 to 71 |
| SEQ ID NO: 2439 | R0235:D12_2 | frame −3 | from 14 to 71 |
| SEQ ID NO: 2440 | R0236:H02_1 | frame 1 | from 1 to 70 |
| SEQ ID NO: 2441 | R0236:H02_2 | frame 2 | from 1 to 111 |
| SEQ ID NO: 2442 | R0236:H02_3 | frame 2 | from 113 to 165 |
| SEQ ID NO: 2443 | R0236:H02_4 | frame 3 | from 1 to 138 |
| SEQ ID NO: 2444 | R0236:H02_5 | frame −2 | from 10 to 110 |
| SEQ ID NO: 2445 | R0236:H02_6 | frame −3 | from 105 to 165 |
| SEQ ID NO: 2446 | R0251:B12_1 | frame 3 | from 1 to 196 |
| SEQ ID NO: 2447 | R0251:B12_2 | frame −2 | from 15 to 128 |
| SEQ ID NO: 2448 | R0251:B12_3 | frame −2 | from 130 to 196 |
| SEQ ID NO: 2449 | R0253:D09_1 | frame 2 | from 1 to 65 |
| SEQ ID NO: 2450 R0253:D09_2 | frame 2 | from 67 to 116 | |
| SEQ ID NO: 2451 | R0253:D09_3 | frame 3 | from 31 to 115 |
| SEQ ID NO: 2452 | R0253:D09_4 | frame −1 | from 1 to 116 |
| SEQ ID NO: 2453 | R0253:D09_5 | frame −3 | from 13 to 66 |
| SEQ ID NO: 2454 | R0254:F10_1 | frame 3 | from 54 to 103 |
| SEQ ID NO: 2455 | R0254:D02_1 | frame 1 | from 1 to 53 |
| SEQ ID NO: 2456 | R0254:D02_2 | frame 1 | from 55 to 135 |
| SEQ ID NO: 2457 | R0254:D02_3 | frame 2 | from 109 to 158 |
| SEQ ID NO: 2458 | R0254:D02_4 | frame −2 | from 1 to 193 |
| SEQ ID NO: 2459 | R0254:D02_5 | frame −3 | from 33 to 90 |
| SEQ ID NO: 2460 | R0238:B06_1 | frame 3 | from 31 to 139 |
| SEQ ID NO: 2461 | R0238:B06_2 | frame −1 | from 1 to 51 |
| SEQ ID NO: 2462 | R0238:B06_3 | frame −2 | from 69 to 119 |
| SEQ ID NO: 2463 | R0238:B06_4 | frame −3 | from 4 to 70 |
| SEQ ID NO: 2464 | R0255:D01_1 | frame 1 | from 9 to 90 |
| SEQ ID NO: 2465 | R0255:D01_2 | frame 1 | from 104 to 168 |
| SEQ ID NO: 2466 | R0255:D01_3 | frame 2 | from 88 to 168 |
| SEQ ID NO: 2467 | R0255:D01_4 | frame −1 | from 1 to 79 |
| SEQ ID NO: 2468 | R0255:D01_5 | frame −1 | from 81 to 130 |
| SEQ ID NO: 2469 | R0255:D01_6 | frame −2 | from 1 to 59 |
| SEQ ID NO: 2470 | R0255:D01_7 | frame −2 | from 62 to 168 |
| SEQ ID NO: 2471 | R0255:D01_8 | frame −3 | from 35 to 91 |
| SEQ ID NO: 2472 | R0255:D01_9 | frame −3 | from 93 to 156 |
| SEQ ID NO: 2473 | R0255:C02_1 | frame 1 | from 1 to 60 |
| SEQ ID NO: 2474 | R0255:C02_2 | frame 3 | from 23 to 96 |
| SEQ ID NO: 2475 | R0255:C02_3 | frame −1 | from 35 to 108 |
| SEQ ID NO: 2476 | R0261:H04_1 | frame 1 | from 100 to 159 |
| SEQ ID NO: 2477 R0261:H04_2 | frame 3 | from 1 to 126 | |
| SEQ ID NO: 2478 R0261:H04_3 | frame −1 | from 1 to 97 | |
| SEQ ID NO: 2479 | R0261:H04_4 | frame −2 | from 1 to 75 |
| SEQ ID NO: 2480 | R0261:H04_5 | frame −2 | from 77 to 128 |
| SEQ ID NO: 2481 | R0261:H04_6 | frame −3 | from 6 to 158 |
| SEQ ID NO: 2482 | R0259:C04_1 | frame 1 | from 16 to 78 |
| SEQ ID NO: 2483 | R0259:C04_2 | frame −2 | from 53 to 139 |
| SEQ ID NO: 2484 | R0259:C06_1 | frame −1 | from 36 to 100 |
| SEQ ID NO: 2485 | R0259:C06_2 | frame −2 | from 124 to 187 |
| SEQ ID NO: 2486 | R0261:H08_1 | frame 1 | from 29 to 102 |
| SEQ ID NO: 2487 | R0261:H08_2 | frame 2 | from 1 to 101 |
| SEQ ID NO: 2488 | R0261:H08_3 | frame 3 | from 1 to 101 |
| SEQ ID NO: 2489 | R0261:H08_4 | frame −2 | from 1 to 74 |
| SEQ ID NO: 2490 | R0261:H08_5 | frame −3 | from 38 to 101 |
| SEQ ID NO: 2491 | R0261:D03_1 | frame 1 | from 44 to 179 |
| SEQ ID NO: 2492 | R0261:D03_2 | frame 2 | from 12 to 90 |
| SEQ ID NO: 2493 | R0261:D03_3 | frame 2 | from 92 to 164 |
| SEQ ID NO: 2494 | R0261:D03_4 | frame 3 | from 40 to 96 |
| SEQ ID NO: 2495 | R0261:D03_5 | frame 3 | from 98 to 186 |
| SEQ ID NO: 2496 | R0261:D03_6 | frame −1 | from 37 to 160 |
| SEQ ID NO: 2497 | R0261:D03_7 | frame −2 | from 22 to 144 |
| SEQ ID NO: 2498 | R0262:C04_1 | frame 1 | from 18 to 75 |
| SEQ ID NO: 2499 | R0262:C04_2 | frame 2 | from 7 to 77 |
| SEQ ID NO: 2500 | R0262:C04_3 | frame −2 | from 67 to 139 |
| SEQ ID NO: 2501 | R0262:C04_4 | frame −3 | from 1 to 88 |
| SEQ ID NO: 2502 | R0264:B08_1 | frame 1 | from 1 to 59 |
| SEQ ID NO: 2503 | R0266:D03_1 | frame 1 | from 1 to 171 |
| SEQ ID NO: 2504 | R0266:D03_2 | frame 2 | from 94 to 193 |
| SEQ ID NO: 2505 | R0266:D03_3 | frame 3 | from 131 to 185 |
| SEQ ID NO: 2506 | R0266:D03_4 | frame −1 | from 89 to 160 |
| SEQ ID NO: 2507 | R0266:D03_5 | frame −3 | from 2 to 59 |
| SEQ ID NO: 2508 | R0266:D03_6 | frame −3 | from 141 to 193 |
| SEQ ID NO: 2509 | R0265:F12_1 | frame 1 | from 75 to 126 |
| SEQ ID NO: 2510 | R0265:F12_2 | frame 2 | from 46 to 160 |
| SEQ ID NO: 2511 | R0265:F12_3 | frame −1 | from 36 to 87 |
| SEQ ID NO: 2512 | R0265:F12_4 | frame −3 | from 78 to 133 |
| SEQ ID NO: 2513 | R0264:C03_1 | frame −2 | from 21 to 77 |
| SEQ ID NO: 2514 | R0264:C04_1 | frame −3 | from 48 to 122 |
| SEQ ID NO: 2515 | R0244:C02_1 | frame 1 | from 1 to 64 |
| SEQ ID NO: 2516 | R0244:C02_2 | frame −1 | from 8 to 107 |
| SEQ ID NO: 2517 | R0244:C02_3 | frame −2 | from 19 to 70 |
| SEQ ID NO: 2518 | R0245:A02_1 | frame 2 | from 12 to 61 |
| SEQ ID NO: 2519 | R0245:A02_2 | frame −3 | from 42 to 92 |
| SEQ ID NO: 2520 | ‘51734.1_gaiger.ABI’_1 | frame 1 | from 12 to 98 |
| SEQ ID NO: 2521 | ‘51734.1_gaiger.ABI’_2 | frame 3 | from 22 to 76 |
| SEQ ID NO: 2522 | ‘51734.1_gaiger.ABI’_3 | frame −2 | from 18 to 137 |
| SEQ ID NO: 2523 | ‘51870.1_gaiger.ABI’_1 | frame 3 | from 33 to 107 |
| SEQ ID NO: 2524 | ‘51870.1_gaiger.ABI’_2 | frame −2 | from 27 to 85 |
| SEQ ID NO: 2525 | ‘51870.1_gaiger.ABI’_3 | frame −3 | from 65 to 115 |
| SEQ ID NO: 2526 | ‘51975.1_gaiger.ABI’_1 | frame 1 | from 17 to 88 |
| SEQ ID NO: 2527 | ‘51975.1_gaiger.ABI’_2 | frame 1 | from 90 to 141 |
| SEQ ID NO: 2528 | ‘51975.1_gaiger.ABI’_3 | frame −1 | from 73 to 147 |
| SEQ ID NO: 2529 | ‘51975.1_gaiger.ABI’_4 | frame −3 | from 1 to 93 |
| SEQ ID NO: 2530 | ‘52260.1_gaiger.ABI’_1 | frame 1 | from 23 to 75 |
| SEQ ID NO: 2531 | ‘52260.1_gaiger.ABI’_2 | frame −1 | from 48 to 105 |
| SEQ ID NO: 2532 | ‘52260.1_gaiger.ABI’_3 | frame −3 | from 13 to 79 |

Table 9 identifies an additional set of particular hematological malignanacy-related cDNA sequences that were obtained using the subtractive library and microarray methods as described above. These sequences, designated SEQ ID NO:2533-SEQ ID NO:9597 in the present specification, are shown in the Table along with the original clone name, and the serial number and filing date of the priority provisional application in which the clone was first described.

TABLE 9
ADDITIONAL POLYNUCLEOTIDES IDENTIFIED BY
SUBTRACTIVE HYBRDIZATION ANALYSES

| Sequence Identifier | Priority | Priority |
|---|---|---|
| Used in the Present | application Ser. | application Filing |
| application | No. | Date |
| SEQ ID NO:2533 | 60/186,126 | 03/01/2000 |
| SEQ ID NO:2534 | 60/186,126 | 03/01/2000 |
| SEQ ID NO:2535 | 60/186,126 | 03/01/2000 |
| SEQ ID NO:2536 | 60/186,126 | 03/01/2000 |
| SEQ ID NO:2537 | 60/186,126 | 03/01/2000 |
| SEQ ID NO:2538 | 60/186,126 | 03/01/2000 |
| SEQ ID NO:2539 | 60/186,126 | 03/01/2000 |
| SEQ ID NO:2540 | 60/186,126 | 03/01/2000 |
| SEQ ID NO:2541 | 60/186,126 | 03/01/2000 |
| SEQ ID NO:2542 | 60/186,126 | 03/01/2000 |
| SEQ ID NO:2543 | 60/186,126 | 03/01/2000 |
| SEQ ID NO:2544 | 60/186,126 | 03/01/2000 |
| SEQ ID NO:2545 | 60/186,126 | 03/01/2000 |
| SEQ ID NO:2546 | 60/186,126 | 03/01/2000 |
| SEQ ID NO:2547 | 60/186,126 | 03/01/2000 |
| SEQ ID NO:2548 | 60/186,126 | 03/01/2000 |
| SEQ ID NO:2549 | 60/186,126 | 03/01/2000 |
| SEQ ID NO:2550 | 60/186,126 | 03/01/2000 |
| SEQ ID NO:2551 | 60/186,126 | 03/01/2000 |
| SEQ ID NO:2552 | 60/186,126 | 03/01/2000 |
| SEQ ID NO:2553 | 60/186,126 | 03/01/2000 |
| SEQ ID NO:2554 | 60/186,126 | 03/01/2000 |
| SEQ ID NO:2555 | 60/186,126 | 03/01/2000 |
| SEQ ID NO:2556 | 60/186,126 | 03/01/2000 |
| SEQ ID NO:2557 | 60/186,126 | 03/01/2000 |
| SEQ ID NO:2558 | 60/186,126 | 03/01/2000 |
| SEQ ID NO:2559 | 60/186,126 | 03/01/2000 |
| SEQ ID NO:2560 | 60/186,126 | 03/01/2000 |
| SEQ ID NO:2561 | 60/186,126 | 03/01/2000 |
| SEQ ID NO:2562 | 60/186,126 | 03/01/2000 |
| SEQ ID NO:2563 | 60/186,126 | 03/01/2000 |
| SEQ ID NO:2564 | 60/186,126 | 03/01/2000 |
| SEQ ID NO:2565 | 60/186,126 | 03/01/2000 |
| SEQ ID NO:2566 | 60/186,126 | 03/01/2000 |
| SEQ ID NO:2567 | 60/186,126 | 03/01/2000 |
| SEQ ID NO:2568 | 60/186,126 | 03/01/2000 |
| SEQ ID NO:2569 | 60/186,126 | 03/01/2000 |
| SEQ ID NO:2570 | 60/186,126 | 03/01/2000 |
| SEQ ID NO:2571 | 60/186,126 | 03/01/2000 |
| SEQ ID NO:2572 | 60/186,126 | 03/01/2000 |
| SEQ ID NO:2573 | 60/186,126 | 03/01/2000 |
| SEQ ID NO:2574 | 60/186,126 | 03/01/2000 |
| SEQ ID NO:2575 | 60/186,126 | 03/01/2000 |
| SEQ ID NO:2576 | 60/186,126 | 03/01/2000 |
| SEQ ID NO:2577 | 60/186,126 | 03/01/2000 |
| SEQ ID NO:2578 | 60/186,126 | 03/01/2000 |
| SEQ ID NO:2579 | 60/186,126 | 03/01/2000 |
| SEQ ID NO:2580 | 60/186,126 | 03/01/2000 |
| SEQ ID NO:2581 | 60/186,126 | 03/01/2000 |
| SEQ ID NO:2582 | 60/186,126 | 03/01/2000 |
| SEQ ID NO:2583 | 60/186,126 | 03/01/2000 |
| SEQ ID NO:2584 | 60/186,126 | 03/01/2000 |
| SEQ ID NO:2585 | 60/186,126 | 03/01/2000 |
| SEQ ID NO:2586 | 60/186,126 | 03/01/2000 |
| SEQ ID NO:2587 | 60/186,126 | 03/01/2000 |
| SEQ ID NO:2588 | 60/186,126 | 03/01/2000 |
| SEQ ID NO:2589 | 60/186,126 | 03/01/2000 |
| SEQ ID NO:2590 | 60/186,126 | 03/01/2000 |
| SEQ ID NO:2591 | 60/186,126 | 03/01/2000 |
| SEQ ID NO:2592 | 60/186,126 | 03/01/2000 |
| SEQ ID NO:2593 | 60/186,126 | 03/01/2000 |
| SEQ ID NO:2594 | 60/186,126 | 03/01/2000 |
| SEQ ID NO:2595 | 60/186,126 | 03/01/2000 |
| SEQ ID NO:2596 | 60/186,126 | 03/01/2000 |
| SEQ ID NO:2597 | 60/186,126 | 03/01/2000 |
| SEQ ID NO:2598 | 60/186,126 | 03/01/2000 |
| SEQ ID NO:2599 | 60/186,126 | 03/01/2000 |
| SEQ ID NO:2600 | 60/186,126 | 03/01/2000 |
| SEQ ID NO:2601 | 60/186,126 | 03/01/2000 |
| SEQ ID NO:2602 | 60/186,126 | 03/01/2000 |
| SEQ ID NO:2603 | 60/186,126 | 03/01/2000 |
| SEQ ID NO:2604 | 60/186,126 | 03/01/2000 |
| SEQ ID NO:2605 | 60/186,126 | 03/01/2000 |
| SEQ ID NO:2606 | 60/186,126 | 03/01/2000 |
| SEQ ID NO:2607 | 60/186,126 | 03/01/2000 |
| SEQ ID NO:2608 | 60/186,126 | 03/01/2000 |
| SEQ ID NO:2609 | 60/186,126 | 03/01/2000 |
| SEQ ID NO:2610 | 60/186,126 | 03/01/2000 |
| SEQ ID NO:2611 | 60/186,126 | 03/01/2000 |
| SEQ ID NO:2612 | 60/186,126 | 03/01/2000 |
| SEQ ID NO:2613 | 60/186,126 | 03/01/2000 |
| SEQ ID NO:2614 | 60/186,126 | 03/01/2000 |
| SEQ ID NO:2615 | 60/186,126 | 03/01/2000 |
| SEQ ID NO:2616 | 60/186,126 | 03/01/2000 |
| SEQ ID NO:2617 | 60/186,126 | 03/01/2000 |
| SEQ ID NO:2618 | 60/186,126 | 03/01/2000 |
| SEQ ID NO:2619 | 60/186,126 | 03/01/2000 |
| SEQ ID NO:2620 | 60/186,126 | 03/01/2000 |
| SEQ ID NO:2621 | 60/186,126 | 03/01/2000 |
| SEQ ID NO:2622 | 60/186,126 | 03/01/2000 |
| SEQ ID NO:2623 | 60/186,126 | 03/01/2000 |
| SEQ ID NO:2624 | 60/186,126 | 03/01/2000 |
| SEQ ID NO:2625 | 60/186,126 | 03/01/2000 |
| SEQ ID NO:2626 | 60/186,126 | 03/01/2000 |
| SEQ ID NO:2627 | 60/186,126 | 03/01/2000 |
| SEQ ID NO:2628 | 60/186,126 | 03/01/2000 |
| SEQ ID NO:2629 | 60/186,126 | 03/01/2000 |
| SEQ ID NO:2630 | 60/186,126 | 03/01/2000 |
| SEQ ID NO:2631 | 60/186,126 | 03/01/2000 |
| SEQ ID NO:2632 | 60/186,126 | 03/01/2000 |
| SEQ ID NO:2633 | 60/186,126 | 03/01/2000 |
| SEQ ID NO:2634 | 60/186,126 | 03/01/2000 |
| SEQ ID NO:2635 | 60/186,126 | 03/01/2000 |
| SEQ ID NO:2636 | 60/186,126 | 03/01/2000 |
| SEQ ID NO:2637 | 60/186,126 | 03/01/2000 |
| SEQ ID NO:2638 | 60/186,126 | 03/01/2000 |
| SEQ ID NO:2639 | 60/186,126 | 03/01/2000 |
| SEQ ID NO:2640 | 60/186,126 | 03/01/2000 |
| SEQ ID NO:2641 | 60/186,126 | 03/01/2000 |
| SEQ ID NO:2642 | 60/186,126 | 03/01/2000 |
| SEQ ID NO:2643 | 60/186,126 | 03/01/2000 |
| SEQ ID NO:2644 | 60/186,126 | 03/01/2000 |
| SEQ ID NO:2645 | 60/186,126 | 03/01/2000 |
| SEQ ID NO:2646 | 60/186,126 | 03/01/2000 |
| SEQ ID NO:2647 | 60/186,126 | 03/01/2000 |
| SEQ ID NO:2648 | 60/186,126 | 03/01/2000 |
| SEQ ID NO:2649 | 60/186,126 | 03/01/2000 |
| SEQ ID NO:2650 | 60/186,126 | 03/01/2000 |
| SEQ ID NO:2651 | 60/186,126 | 03/01/2000 |
| SEQ ID NO:2652 | 60/186,126 | 03/01/2000 |
| SEQ ID NO:2653 | 60/186,126 | 03/01/2000 |
| SEQ ID NO:2654 | 60/186,126 | 03/01/2000 |
| SEQ ID NO:2655 | 60/186,126 | 03/01/2000 |
| SEQ ID NO:2656 | 60/186,126 | 03/01/2000 |
| SEQ ID NO:2657 | 60/186,126 | 03/01/2000 |
| SEQ ID NO:2658 | 60/186,126 | 03/01/2000 |
| SEQ ID NO:2659 | 60/186,126 | 03/01/2000 |
| SEQ ID NO:2660 | 60/186,126 | 03/01/2000 |
| SEQ ID NO:2661 | 60/186,126 | 03/01/2000 |
| SEQ ID NO:2662 | 60/186,126 | 03/01/2000 |
| SEQ ID NO:2663 | 60/186,126 | 03/01/2000 |
| SEQ ID NO:2664 | 60/186,126 | 03/01/2000 |
| SEQ ID NO:2665 | 60/186,126 | 03/01/2000 |
| SEQ ID NO:2666 | 60/186,126 | 03/01/2000 |
| SEQ ID NO:2667 | 60/186,126 | 03/01/2000 |
| SEQ ID NO:2668 | 60/186,126 | 03/01/2000 |
| SEQ ID NO:2669 | 60/186,126 | 03/01/2000 |
| SEQ ID NO:2670 | 60/186,126 | 03/01/2000 |
| SEQ ID NO:2671 | 60/186,126 | 03/01/2000 |
| SEQ ID NO:2672 | 60/186,126 | 03/01/2000 |
| SEQ ID NO:2673 | 60/186,126 | 03/01/2000 |
| SEQ ID NO:2674 | 60/186,126 | 03/01/2000 |
| SEQ ID NO:2675 | 60/186,126 | 03/01/2000 |
| SEQ ID NO:2676 | 60/186,126 | 03/01/2000 |
| SEQ ID NO:2677 | 60/186,126 | 03/01/2000 |
| SEQ ID NO:2678 | 60/186,126 | 03/01/2000 |
| SEQ ID NO:2679 | 60/186,126 | 03/01/2000 |
| SEQ ID NO:2680 | 60/186,126 | 03/01/2000 |
| SEQ ID NO:2681 | 60/186,126 | 03/01/2000 |
| SEQ ID NO:2682 | 60/186,126 | 03/01/2000 |
| SEQ ID NO:2683 | 60/186,126 | 03/01/2000 |
| SEQ ID NO:2684 | 60/186,126 | 03/01/2000 |
| SEQ ID NO:2685 | 60/186,126 | 03/01/2000 |
| SEQ ID NO:2686 | 60/186,126 | 03/01/2000 |
| SEQ ID NO:2687 | 60/186,126 | 03/01/2000 |
| SEQ ID NO:2688 | 60/186,126 | 03/01/2000 |
| SEQ ID NO:2689 | 60/186,126 | 03/01/2000 |
| SEQ ID NO:2690 | 60/186,126 | 03/01/2000 |
| SEQ ID NO:2691 | 60/186,126 | 03/01/2000 |
| SEQ ID NO:2692 | 60/186,126 | 03/01/2000 |
| SEQ ID NO:2693 | 60/186,126 | 03/01/2000 |
| SEQ ID NO:2694 | 60/186,126 | 03/01/2000 |
| SEQ ID NO:2695 | 60/186,126 | 03/01/2000 |
| SEQ ID NO:2696 | 60/186,126 | 03/01/2000 |
| SEQ ID NO:2697 | 60/186,126 | 03/01/2000 |
| SEQ ID NO:2698 | 60/186,126 | 03/01/2000 |
| SEQ ID NO:2699 | 60/186,126 | 03/01/2000 |
| SEQ ID NO:2700 | 60/186,126 | 03/01/2000 |
| SEQ ID NO:2701 | 60/186,126 | 03/01/2000 |
| SEQ ID NO:2702 | 60/186,126 | 03/01/2000 |
| SEQ ID NO:2703 | 60/186,126 | 03/01/2000 |
| SEQ ID NO:2704 | 60/186,126 | 03/01/2000 |
| SEQ ID NO:2705 | 60/186,126 | 03/01/2000 |
| SEQ ID NO:2706 | 60/186,126 | 03/01/2000 |
| SEQ ID NO:2707 | 60/186,126 | 03/01/2000 |
| SEQ ID NO:2708 | 60/186,126 | 03/01/2000 |
| SEQ ID NO:2709 | 60/186,126 | 03/01/2000 |
| SEQ ID NO:2710 | 60/186,126 | 03/01/2000 |
| SEQ ID NO:2711 | 60/186,126 | 03/01/2000 |
| SEQ ID NO:2712 | 60/186,126 | 03/01/2000 |
| SEQ ID NO:2713 | 60/186,126 | 03/01/2000 |
| SEQ ID NO:2714 | 60/186,126 | 03/01/2000 |
| SEQ ID NO:2715 | 60/186,126 | 03/01/2000 |
| SEQ ID NO:2716 | 60/186,126 | 03/01/2000 |
| SEQ ID NO:2717 | 60/186,126 | 03/01/2000 |
| SEQ ID NO:2718 | 60/186,126 | 03/01/2000 |
| SEQ ID NO:2719 | 60/186,126 | 03/01/2000 |
| SEQ ID NO:2720 | 60/186,126 | 03/01/2000 |
| SEQ ID NO:2721 | 60/186,126 | 03/01/2000 |
| SEQ ID NO:2722 | 60/186,126 | 03/01/2000 |
| SEQ ID NO:2723 | 60/186,126 | 03/01/2000 |
| SEQ ID NO:2724 | 60/186,126 | 03/01/2000 |
| SEQ ID NO:2725 | 60/186,126 | 03/01/2000 |
| SEQ ID NO:2726 | 60/186,126 | 03/01/2000 |
| SEQ ID NO:2727 | 60/186,126 | 03/01/2000 |
| SEQ ID NO:2728 | 60/186,126 | 03/01/2000 |
| SEQ ID NO:2729 | 60/186,126 | 03/01/2000 |
| SEQ ID NO:2730 | 60/186,126 | 03/01/2000 |
| SEQ ID NO:2731 | 60/186,126 | 03/01/2000 |
| SEQ ID NO:2732 | 60/186,126 | 03/01/2000 |
| SEQ ID NO:2733 | 60/186,126 | 03/01/2000 |
| SEQ ID NO:2734 | 60/186,126 | 03/01/2000 |
| SEQ ID NO:2735 | 60/186,126 | 03/01/2000 |
| SEQ ID NO:2736 | 60/186,126 | 03/01/2000 |
| SEQ ID NO:2737 | 60/186,126 | 03/01/2000 |
| SEQ ID NO:2738 | 60/186,126 | 03/01/2000 |
| SEQ ID NO:2739 | 60/186,126 | 03/01/2000 |
| SEQ ID NO:2740 | 60/186,126 | 03/01/2000 |
| SEQ ID NO:2741 | 60/186,126 | 03/01/2000 |
| SEQ ID NO:2742 | 60/186,126 | 03/01/2000 |
| SEQ ID NO:2743 | 60/186,126 | 03/01/2000 |
| SEQ ID NO:2744 | 60/186,126 | 03/01/2000 |
| SEQ ID NO:2745 | 60/186,126 | 03/01/2000 |
| SEQ ID NO:2746 | 60/186,126 | 03/01/2000 |
| SEQ ID NO:2747 | 60/186,126 | 03/01/2000 |
| SEQ ID NO:2748 | 60/186,126 | 03/01/2000 |
| SEQ ID NO:2749 | 60/186,126 | 03/01/2000 |
| SEQ ID NO:2750 | 60/186,126 | 03/01/2000 |
| SEQ ID NO:2751 | 60/186,126 | 03/01/2000 |
| SEQ ID NO:2752 | 60/186,126 | 03/01/2000 |
| SEQ ID NO:2753 | 60/186,126 | 03/01/2000 |
| SEQ ID NO:2754 | 60/186,126 | 03/01/2000 |
| SEQ ID NO:2755 | 60/186,126 | 03/01/2000 |
| SEQ ID NO:2756 | 60/186,126 | 03/01/2000 |
| SEQ ID NO:2757 | 60/186,126 | 03/01/2000 |
| SEQ ID NO:2758 | 60/186,126 | 03/01/2000 |
| SEQ ID NO:2759 | 60/186,126 | 03/01/2000 |
| SEQ ID NO:2760 | 60/186,126 | 03/01/2000 |
| SEQ ID NO:2761 | 60/186,126 | 03/01/2000 |
| SEQ ID NO:2762 | 60/186,126 | 03/01/2000 |
| SEQ ID NO:2763 | 60/186,126 | 03/01/2000 |
| SEQ ID NO:2764 | 60/186,126 | 03/01/2000 |
| SEQ ID NO:2765 | 60/186,126 | 03/01/2000 |
| SEQ ID NO:2766 | 60/186,126 | 03/01/2000 |
| SEQ ID NO:2767 | 60/186,126 | 03/01/2000 |
| SEQ ID NO:2768 | 60/186,126 | 03/01/2000 |
| SEQ ID NO:2769 | 60/186,126 | 03/01/2000 |
| SEQ ID NO:2770 | 60/186,126 | 03/01/2000 |
| SEQ ID NO:2771 | 60/186,126 | 03/01/2000 |
| SEQ ID NO:2772 | 60/186,126 | 03/01/2000 |
| SEQ ID NO:2773 | 60/186,126 | 03/01/2000 |
| SEQ ID NO:2774 | 60/186,126 | 03/01/2000 |
| SEQ ID NO:2775 | 60/186,126 | 03/01/2000 |
| SEQ ID NO:2776 | 60/186,126 | 03/01/2000 |
| SEQ ID NO:2777 | 60/186,126 | 03/01/2000 |
| SEQ ID NO:2778 | 60/186,126 | 03/01/2000 |
| SEQ ID NO:2779 | 60/186,126 | 03/01/2000 |
| SEQ ID NO:2780 | 60/186,126 | 03/01/2000 |
| SEQ ID NO:2781 | 60/186,126 | 03/01/2000 |
| SEQ ID NO:2782 | 60/186,126 | 03/01/2000 |
| SEQ ID NO:2783 | 60/186,126 | 03/01/2000 |
| SEQ ID NO:2784 | 60/186,126 | 03/01/2000 |
| SEQ ID NO:2785 | 60/186,126 | 03/01/2000 |
| SEQ ID NO:2786 | 60/186,126 | 03/01/2000 |
| SEQ ID NO:2787 | 60/186,126 | 03/01/2000 |
| SEQ ID NO:2788 | 60/186,126 | 03/01/2000 |
| SEQ ID NO:2789 | 60/186,126 | 03/01/2000 |
| SEQ ID NO:2790 | 60/186,126 | 03/01/2000 |
| SEQ ID NO:2791 | 60/186,126 | 03/01/2000 |
| SEQ ID NO:2792 | 60/186,126 | 03/01/2000 |
| SEQ ID NO:2793 | 60/186,126 | 03/01/2000 |
| SEQ ID NO:2794 | 60/186,126 | 03/01/2000 |
| SEQ ID NO:2795 | 60/186,126 | 03/01/2000 |
| SEQ ID NO:2796 | 60/186,126 | 03/01/2000 |
| SEQ ID NO:2797 | 60/186,126 | 03/01/2000 |
| SEQ ID NO:2798 | 60/186,126 | 03/01/2000 |
| SEQ ID NO:2799 | 60/186,126 | 03/01/2000 |
| SEQ ID NO:2800 | 60/186,126 | 03/01/2000 |
| SEQ ID NO:2801 | 60/186,126 | 03/01/2000 |
| SEQ ID NO:2802 | 60/186,126 | 03/01/2000 |
| SEQ ID NO:2803 | 60/186,126 | 03/01/2000 |
| SEQ ID NO:2804 | 60/186,126 | 03/01/2000 |
| SEQ ID NO:2805 | 60/186,126 | 03/01/2000 |
| SEQ ID NO:2806 | 60/186,126 | 03/01/2000 |
| SEQ ID NO:2807 | 60/186,126 | 03/01/2000 |
| SEQ ID NO:2808 | 60/186,126 | 03/01/2000 |
| SEQ ID NO:2809 | 60/186,126 | 03/01/2000 |
| SEQ ID NO:2810 | 60/186,126 | 03/01/2000 |
| SEQ ID NO:2811 | 60/186,126 | 03/01/2000 |
| SEQ ID NO:2812 | 60/186,126 | 03/01/2000 |
| SEQ ID NO:2813 | 60/186,126 | 03/01/2000 |
| SEQ ID NO:2814 | 60/186,126 | 03/01/2000 |
| SEQ ID NO:2815 | 60/186,126 | 03/01/2000 |
| SEQ ID NO:2816 | 60/186,126 | 03/01/2000 |
| SEQ ID NO:2817 | 60/186,126 | 03/01/2000 |
| SEQ ID NO:2818 | 60/186,126 | 03/01/2000 |
| SEQ ID NO:2819 | 60/186,126 | 03/01/2000 |
| SEQ ID NO:2820 | 60/186,126 | 03/01/2000 |
| SEQ ID NO:2821 | 60/186,126 | 03/01/2000 |
| SEQ ID NO:2822 | 60/186,126 | 03/01/2000 |
| SEQ ID NO:2823 | 60/186,126 | 03/01/2000 |
| SEQ ID NO:2824 | 60/186,126 | 03/01/2000 |
| SEQ ID NO:2825 | 60/186,126 | 03/01/2000 |
| SEQ ID NO:2826 | 60/186,126 | 03/01/2000 |
| SEQ ID NO:2827 | 60/186,126 | 03/01/2000 |
| SEQ ID NO:2828 | 60/186,126 | 03/01/2000 |
| SEQ ID NO:2829 | 60/186,126 | 03/01/2000 |
| SEQ ID NO:2830 | 60/186,126 | 03/01/2000 |
| SEQ ID NO:2831 | 60/186,126 | 03/01/2000 |
| SEQ ID NO:2832 | 60/186,126 | 03/01/2000 |
| SEQ ID NO:2833 | 60/186,126 | 03/01/2000 |
| SEQ ID NO:2834 | 60/186,126 | 03/01/2000 |
| SEQ ID NO:2835 | 60/186,126 | 03/01/2000 |
| SEQ ID NO:2836 | 60/186,126 | 03/01/2000 |
| SEQ ID NO:2837 | 60/186,126 | 03/01/2000 |
| SEQ ID NO:2838 | 60/186,126 | 03/01/2000 |
| SEQ ID NO:2839 | 60/186,126 | 03/01/2000 |
| SEQ ID NO:2840 | 60/186,126 | 03/01/2000 |
| SEQ ID NO:2841 | 60/186,126 | 03/01/2000 |
| SEQ ID NO:2842 | 60/186,126 | 03/01/2000 |
| SEQ ID NO:2843 | 60/186,126 | 03/01/2000 |
| SEQ ID NO:2844 | 60/186,126 | 03/01/2000 |
| SEQ ID NO:2845 | 60/186,126 | 03/01/2000 |
| SEQ ID NO:2846 | 60/186,126 | 03/01/2000 |
| SEQ ID NO:2847 | 60/186,126 | 03/01/2000 |
| SEQ ID NO:2848 | 60/186,126 | 03/01/2000 |
| SEQ ID NO:2849 | 60/186,126 | 03/01/2000 |
| SEQ ID NO:2850 | 60/186,126 | 03/01/2000 |
| SEQ ID NO:2851 | 60/186,126 | 03/01/2000 |
| SEQ ID NO:2852 | 60/186,126 | 03/01/2000 |
| SEQ ID NO:2853 | 60/186,126 | 03/01/2000 |
| SEQ ID NO:2854 | 60/186,126 | 03/01/2000 |
| SEQ ID NO:2855 | 60/186,126 | 03/01/2000 |
| SEQ ID NO:2856 | 60/186,126 | 03/01/2000 |
| SEQ ID NO:2857 | 60/186,126 | 03/01/2000 |
| SEQ ID NO:2858 | 60/186,126 | 03/01/2000 |
| SEQ ID NO:2859 | 60/186,126 | 03/01/2000 |
| SEQ ID NO:2860 | 60/186,126 | 03/01/2000 |
| SEQ ID NO:2861 | 60/186,126 | 03/01/2000 |
| SEQ ID NO:2862 | 60/186,126 | 03/01/2000 |
| SEQ ID NO:2863 | 60/186,126 | 03/01/2000 |
| SEQ ID NO:2864 | 60/186,126 | 03/01/2000 |
| SEQ ID NO:2865 | 60/186,126 | 03/01/2000 |
| SEQ ID NO:2866 | 60/186,126 | 03/01/2000 |
| SEQ ID NO:2867 | 60/186,126 | 03/01/2000 |
| SEQ ID NO:2868 | 60/186,126 | 03/01/2000 |
| SEQ ID NO:2869 | 60/186,126 | 03/01/2000 |
| SEQ ID NO:2870 | 60/186,126 | 03/01/2000 |
| SEQ ID NO:2871 | 60/186,126 | 03/01/2000 |
| SEQ ID NO:2872 | 60/186,126 | 03/01/2000 |
| SEQ ID NO:2873 | 60/186,126 | 03/01/2000 |
| SEQ ID NO:2874 | 60/186,126 | 03/01/2000 |
| SEQ ID NO:2875 | 60/186,126 | 03/01/2000 |
| SEQ ID NO:2876 | 60/186,126 | 03/01/2000 |
| SEQ ID NO:2877 | 60/186,126 | 03/01/2000 |
| SEQ ID NO:2878 | 60/186,126 | 03/01/2000 |
| SEQ ID NO:2879 | 60/186,126 | 03/01/2000 |
| SEQ ID NO:2880 | 60/186,126 | 03/01/2000 |
| SEQ ID NO:2881 | 60/186,126 | 03/01/2000 |
| SEQ ID NO:2882 | 60/186,126 | 03/01/2000 |
| SEQ ID NO:2883 | 60/186,126 | 03/01/2000 |
| SEQ ID NO:2884 | 60/186,126 | 03/01/2000 |
| SEQ ID NO:2885 | 60/186,126 | 03/01/2000 |
| SEQ ID NO:2886 | 60/186,126 | 03/01/2000 |
| SEQ ID NO:2887 | 60/186,126 | 03/01/2000 |
| SEQ ID NO:2888 | 60/186,126 | 03/01/2000 |
| SEQ ID NO:2889 | 60/186,126 | 03/01/2000 |
| SEQ ID NO:2890 | 60/186,126 | 03/01/2000 |
| SEQ ID NO:2891 | 60/186,126 | 03/01/2000 |
| SEQ ID NO:2892 | 60/186,126 | 03/01/2000 |
| SEQ ID NO:2893 | 60/186,126 | 03/01/2000 |
| SEQ ID NO:2894 | 60/186,126 | 03/01/2000 |
| SEQ ID NO:2895 | 60/186,126 | 03/01/2000 |
| SEQ ID NO:2896 | 60/186,126 | 03/01/2000 |
| SEQ ID NO:2897 | 60/186,126 | 03/01/2000 |
| SEQ ID NO:2898 | 60/186,126 | 03/01/2000 |
| SEQ ID NO:2899 | 60/186,126 | 03/01/2000 |
| SEQ ID NO:2900 | 60/186,126 | 03/01/2000 |
| SEQ ID NO:2901 | 60/186,126 | 03/01/2000 |
| SEQ ID NO:2902 | 60/186,126 | 03/01/2000 |
| SEQ ID NO:2903 | 60/186,126 | 03/01/2000 |
| SEQ ID NO:2904 | 60/186,126 | 03/01/2000 |
| SEQ ID NO:2905 | 60/186,126 | 03/01/2000 |
| SEQ ID NO:2906 | 60/186,126 | 03/01/2000 |
| SEQ ID NO:2907 | 60/186,126 | 03/01/2000 |
| SEQ ID NO:2908 | 60/186,126 | 03/01/2000 |
| SEQ ID NO:2909 | 60/186,126 | 03/01/2000 |
| SEQ ID NO:2910 | 60/186,126 | 03/01/2000 |
| SEQ ID NO:2911 | 60/186,126 | 03/01/2000 |
| SEQ ID NO:2912 | 60/186,126 | 03/01/2000 |
| SEQ ID NO:2913 | 60/186,126 | 03/01/2000 |
| SEQ ID NO:2914 | 60/190,479 | 03/17/2000 |
| SEQ ID NO:2915 | 60/190,479 | 03/17/2000 |
| SEQ ID NO:2916 | 60/190,479 | 03/17/2000 |
| SEQ ID NO:2917 | 60/190,479 | 03/17/2000 |
| SEQ ID NO:2918 | 60/190,479 | 03/17/2000 |
| SEQ ID NO:2919 | 60/190,479 | 03/17/2000 |
| SEQ ID NO:2920 | 60/190,479 | 03/17/2000 |
| SEQ ID NO:2921 | 60/190,479 | 03/17/2000 |
| SEQ ID NO:2922 | 60/190,479 | 03/17/2000 |
| SEQ ID NO:2923 | 60/190,479 | 03/17/2000 |
| SEQ ID NO:2924 | 60/190,479 | 03/17/2000 |
| SEQ ID NO:2925 | 60/190,479 | 03/17/2000 |
| SEQ ID NO:2926 | 60/190,479 | 03/17/2000 |
| SEQ ID NO:2927 | 60/190,479 | 03/17/2000 |
| SEQ ID NO:2928 | 60/190,479 | 03/17/2000 |
| SEQ ID NO:2929 | 60/190,479 | 03/17/2000 |
| SEQ ID NO:2930 | 60/190,479 | 03/17/2000 |
| SEQ ID NO:2931 | 60/190,479 | 03/17/2000 |
| SEQ ID NO:2932 | 60/190,479 | 03/17/2000 |
| SEQ ID NO:2933 | 60/190,479 | 03/17/2000 |
| SEQ ID NO:2934 | 60/190,479 | 03/17/2000 |
| SEQ ID NO:2935 | 60/190,479 | 03/17/2000 |
| SEQ ID NO:2936 | 60/190,479 | 03/17/2000 |
| SEQ ID NO:2937 | 60/190,479 | 03/17/2000 |
| SEQ ID NO:2938 | 60/190,479 | 03/17/2000 |
| SEQ ID NO:2939 | 60/190,479 | 03/17/2000 |
| SEQ ID NO:2940 | 60/190,479 | 03/17/2000 |
| SEQ ID NO:2941 | 60/190,479 | 03/17/2000 |
| SEQ ID NO:2942 | 60/190,479 | 03/17/2000 |
| SEQ ID NO:2943 | 60/190,479 | 03/17/2000 |
| SEQ ID NO:2944 | 60/190,479 | 03/17/2000 |
| SEQ ID NO:2945 | 60/190,479 | 03/17/2000 |
| SEQ ID NO:2946 | 60/190,479 | 03/17/2000 |
| SEQ ID NO:2947 | 60/190,479 | 03/17/2000 |
| SEQ ID NO:2948 | 60/190,479 | 03/17/2000 |
| SEQ ID NO:2949 | 60/190,479 | 03/17/2000 |
| SEQ ID NO:2950 | 60/190,479 | 03/17/2000 |
| SEQ ID NO:2951 | 60/190,479 | 03/17/2000 |
| SEQ ID NO:2952 | 60/190,479 | 03/17/2000 |
| SEQ ID NO:2953 | 60/190,479 | 03/17/2000 |
| SEQ ID NO:2954 | 60/190,479 | 03/17/2000 |
| SEQ ID NO:2955 | 60/190,479 | 03/17/2000 |
| SEQ ID NO:2956 | 60/190,479 | 03/17/2000 |
| SEQ ID NO:2957 | 60/190,479 | 03/17/2000 |
| SEQ ID NO:2958 | 60/190,479 | 03/17/2000 |
| SEQ ID NO:2959 | 60/190,479 | 03/17/2000 |
| SEQ ID NO:2960 | 60/190,479 | 03/17/2000 |
| SEQ ID NO:2961 | 60/190,479 | 03/17/2000 |
| SEQ ID NO:2962 | 60/190,479 | 03/17/2000 |
| SEQ ID NO:2963 | 60/190,479 | 03/17/2000 |
| SEQ ID NO:2964 | 60/190,479 | 03/17/2000 |
| SEQ ID NO:2965 | 60/190,479 | 03/17/2000 |
| SEQ ID NO:2966 | 60/190,479 | 03/17/2000 |
| SEQ ID NO:2967 | 60/190,479 | 03/17/2000 |
| SEQ ID NO:2968 | 60/190,479 | 03/17/2000 |
| SEQ ID NO:2969 | 60/190,479 | 03/17/2000 |
| SEQ ID NO:2970 | 60/190,479 | 03/17/2000 |
| SEQ ID NO:2971 | 60/190,479 | 03/17/2000 |
| SEQ ID NO:2972 | 60/190,479 | 03/17/2000 |
| SEQ ID NO:2973 | 60/190,479 | 03/17/2000 |
| SEQ ID NO:2974 | 60/190,479 | 03/17/2000 |
| SEQ ID NO:2975 | 60/190,479 | 03/17/2000 |
| SEQ ID NO:2976 | 60/190,479 | 03/17/2000 |
| SEQ ID NO:2977 | 60/190,479 | 03/17/2000 |
| SEQ ID NO:2978 | 60/190,479 | 03/17/2000 |
| SEQ ID NO:2979 | 60/190,479 | 03/17/2000 |
| SEQ ID NO:2980 | 60/190,479 | 03/17/2000 |
| SEQ ID NO:2981 | 60/190,479 | 03/17/2000 |
| SEQ ID NO:2982 | 60/190,479 | 03/17/2000 |
| SEQ ID NO:2983 | 60/190,479 | 03/17/2000 |
| SEQ ID NO:2984 | 60/190,479 | 03/17/2000 |
| SEQ ID NO:2985 | 60/190,479 | 03/17/2000 |
| SEQ ID NO:2986 | 60/190,479 | 03/17/2000 |
| SEQ ID NO:2987 | 60/190,479 | 03/17/2000 |
| SEQ ID NO:2988 | 60/190,479 | 03/17/2000 |
| SEQ ID NO:2989 | 60/190,479 | 03/17/2000 |
| SEQ ID NO:2990 | 60/190,479 | 03/17/2000 |
| SEQ ID NO:2991 | 60/190,479 | 03/17/2000 |
| SEQ ID NO:2992 | 60/190,479 | 03/17/2000 |
| SEQ ID NO:2993 | 60/190,479 | 03/17/2000 |
| SEQ ID NO:2994 | 60/190,479 | 03/17/2000 |
| SEQ ID NO:2995 | 60/190,479 | 03/17/2000 |
| SEQ ID NO:2996 | 60/190,479 | 03/17/2000 |
| SEQ ID NO:2997 | 60/190,479 | 03/17/2000 |
| SEQ ID NO:2998 | 60/190,479 | 03/17/2000 |
| SEQ ID NO:2999 | 60/190,479 | 03/17/2000 |
| SEQ ID NO:3000 | 60/190,479 | 03/17/2000 |
| SEQ ID NO:3001 | 60/190,479 | 03/17/2000 |
| SEQ ID NO:3002 | 60/190,479 | 03/17/2000 |
| SEQ ID NO:3003 | 60/190,479 | 03/17/2000 |
| SEQ ID NO:3004 | 60/190,479 | 03/17/2000 |
| SEQ ID NO:3005 | 60/190,479 | 03/17/2000 |
| SEQ ID NO:3006 | 60/190,479 | 03/17/2000 |
| SEQ ID NO:3007 | 60/190,479 | 03/17/2000 |
| SEQ ID NO:3008 | 60/190,479 | 03/17/2000 |
| SEQ ID NO:3009 | 60/190,479 | 03/17/2000 |
| SEQ ID NO:3010 | 60/190,479 | 03/17/2000 |
| SEQ ID NO:3011 | 60/190,479 | 03/17/2000 |
| SEQ ID NO:3012 | 60/190,479 | 03/17/2000 |
| SEQ ID NO:3013 | 60/190,479 | 03/17/2000 |
| SEQ ID NO:3014 | 60/190,479 | 03/17/2000 |
| SEQ ID NO:3015 | 60/190,479 | 03/17/2000 |
| SEQ ID NO:3016 | 60/190,479 | 03/17/2000 |
| SEQ ID NO:3017 | 60/190,479 | 03/17/2000 |
| SEQ ID NO:3018 | 60/190,479 | 03/17/2000 |
| SEQ ID NO:3019 | 60/190,479 | 03/17/2000 |
| SEQ ID NO:3020 | 60/190,479 | 03/17/2000 |
| SEQ ID NO:3021 | 60/190,479 | 03/17/2000 |
| SEQ ID NO:3022 | 60/190,479 | 03/17/2000 |
| SEQ ID NO:3023 | 60/190,479 | 03/17/2000 |
| SEQ ID NO:3024 | 60/190,479 | 03/17/2000 |
| SEQ ID NO:3025 | 60/190,479 | 03/17/2000 |
| SEQ ID NO:3026 | 60/190,479 | 03/17/2000 |
| SEQ ID NO:3027 | 60/190,479 | 03/17/2000 |
| SEQ ID NO:3028 | 60/190,479 | 03/17/2000 |
| SEQ ID NO:3029 | 60/190,479 | 03/17/2000 |
| SEQ ID NO:3030 | 60/190,479 | 03/17/2000 |
| SEQ ID NO:3031 | 60/190,479 | 03/17/2000 |
| SEQ ID NO:3032 | 60/190,479 | 03/17/2000 |
| SEQ ID NO:3033 | 60/190,479 | 03/17/2000 |
| SEQ ID NO:3034 | 60/190,479 | 03/17/2000 |
| SEQ ID NO:3035 | 60/190,479 | 03/17/2000 |
| SEQ ID NO:3036 | 60/190,479 | 03/17/2000 |
| SEQ ID NO:3037 | 60/190,479 | 03/17/2000 |
| SEQ ID NO:3038 | 60/190,479 | 03/17/2000 |
| SEQ ID NO:3039 | 60/190,479 | 03/17/2000 |
| SEQ ID NO:3040 | 60/190,479 | 03/17/2000 |
| SEQ ID NO:3041 | 60/190,479 | 03/17/2000 |
| SEQ ID NO:3042 | 60/190,479 | 03/17/2000 |
| SEQ ID NO:3043 | 60/190,479 | 03/17/2000 |
| SEQ ID NO:3044 | 60/190,479 | 03/17/2000 |
| SEQ ID NO:3045 | 60/190,479 | 03/17/2000 |
| SEQ ID NO:3046 | 60/190,479 | 03/17/2000 |
| SEQ ID NO:3047 | 60/190,479 | 03/17/2000 |
| SEQ ID NO:3048 | 60/190,479 | 03/17/2000 |
| SEQ ID NO:3049 | 60/190,479 | 03/17/2000 |
| SEQ ID NO:3050 | 60/190,479 | 03/17/2000 |
| SEQ ID NO:3051 | 60/190,479 | 03/17/2000 |
| SEQ ID NO:3052 | 60/190,479 | 03/17/2000 |
| SEQ ID NO:3053 | 60/190,479 | 03/17/2000 |
| SEQ ID NO:3054 | 60/190,479 | 03/17/2000 |
| SEQ ID NO:3055 | 60/190,479 | 03/17/2000 |
| SEQ ID NO:3056 | 60/190,479 | 03/17/2000 |
| SEQ ID NO:3057 | 60/190,479 | 03/17/2000 |
| SEQ ID NO:3058 | 60/190,479 | 03/17/2000 |
| SEQ ID NO:3059 | 60/190,479 | 03/17/2000 |
| SEQ ID NO:3060 | 60/190,479 | 03/17/2000 |
| SEQ ID NO:3061 | 60/190,479 | 03/17/2000 |
| SEQ ID NO:3062 | 60/190,479 | 03/17/2000 |
| SEQ ID NO:3063 | 60/190,479 | 03/17/2000 |
| SEQ ID NO:3064 | 60/190,479 | 03/17/2000 |
| SEQ ID NO:3065 | 60/190,479 | 03/17/2000 |
| SEQ ID NO:3066 | 60/190,479 | 03/17/2000 |
| SEQ ID NO:3067 | 60/190,479 | 03/17/2000 |
| SEQ ID NO:3068 | 60/190,479 | 03/17/2000 |
| SEQ ID NO:3069 | 60/190,479 | 03/17/2000 |
| SEQ ID NO:3070 | 60/190,479 | 03/17/2000 |
| SEQ ID NO:3071 | 60/190,479 | 03/17/2000 |
| SEQ ID NO:3072 | 60/190,479 | 03/17/2000 |
| SEQ ID NO:3073 | 60/190,479 | 03/17/2000 |
| SEQ ID NO:3074 | 60/190,479 | 03/17/2000 |
| SEQ ID NO:3075 | 60/190,479 | 03/17/2000 |
| SEQ ID NO:3076 | 60/190,479 | 03/17/2000 |
| SEQ ID NO:3077 | 60/190,479 | 03/17/2000 |
| SEQ ID NO:3078 | 60/190,479 | 03/17/2000 |
| SEQ ID NO:3079 | 60/190,479 | 03/17/2000 |
| SEQ ID NO:3080 | 60/190,479 | 03/17/2000 |
| SEQ ID NO:3081 | 60/190,479 | 03/17/2000 |
| SEQ ID NO:3082 | 60/190,479 | 03/17/2000 |
| SEQ ID NO:3083 | 60/190,479 | 03/17/2000 |
| SEQ ID NO:3084 | 60/190,479 | 03/17/2000 |
| SEQ ID NO:3085 | 60/190,479 | 03/17/2000 |
| SEQ ID NO:3086 | 60/190,479 | 03/17/2000 |
| SEQ ID NO:3087 | 60/190,479 | 03/17/2000 |
| SEQ ID NO:3088 | 60/190,479 | 03/17/2000 |
| SEQ ID NO:3089 | 60/190,479 | 03/17/2000 |
| SEQ ID NO:3090 | 60/190,479 | 03/17/2000 |
| SEQ ID NO:3091 | 60/190,479 | 03/17/2000 |
| SEQ ID NO:3092 | 60/190,479 | 03/17/2000 |
| SEQ ID NO:3093 | 60/190,479 | 03/17/2000 |
| SEQ ID NO:3094 | 60/190,479 | 03/17/2000 |
| SEQ ID NO:3095 | 60/190,479 | 03/17/2000 |
| SEQ ID NO:3096 | 60/190,479 | 03/17/2000 |
| SEQ ID NO:3097 | 60/190,479 | 03/17/2000 |
| SEQ ID NO:3098 | 60/190,479 | 03/17/2000 |
| SEQ ID NO:3099 | 60/190,479 | 03/17/2000 |
| SEQ ID NO:3100 | 60/190,479 | 03/17/2000 |
| SEQ ID NO:3101 | 60/190,479 | 03/17/2000 |
| SEQ ID NO:3102 | 60/190,479 | 03/17/2000 |
| SEQ ID NO:3103 | 60/190,479 | 03/17/2000 |
| SEQ ID NO:3104 | 60/190,479 | 03/17/2000 |
| SEQ ID NO:3105 | 60/190,479 | 03/17/2000 |
| SEQ ID NO:3106 | 60/190,479 | 03/17/2000 |
| SEQ ID NO:3107 | 60/190,479 | 03/17/2000 |
| SEQ ID NO:3108 | 60/190,479 | 03/17/2000 |
| SEQ ID NO:3109 | 60/190,479 | 03/17/2000 |
| SEQ ID NO:3110 | 60/190,479 | 03/17/2000 |
| SEQ ID NO:3111 | 60/190,479 | 03/17/2000 |
| SEQ ID NO:3112 | 60/190,479 | 03/17/2000 |
| SEQ ID NO:3113 | 60/190,479 | 03/17/2000 |
| SEQ ID NO:3114 | 60/190,479 | 03/17/2000 |
| SEQ ID NO:3115 | 60/190,479 | 03/17/2000 |
| SEQ ID NO:3116 | 60/190,479 | 03/17/2000 |
| SEQ ID NO:3117 | 60/190,479 | 03/17/2000 |
| SEQ ID NO:3118 | 60/190,479 | 03/17/2000 |
| SEQ ID NO:3119 | 60/190,479 | 03/17/2000 |
| SEQ ID NO:3120 | 60/190,479 | 03/17/2000 |
| SEQ ID NO:3121 | 60/190,479 | 03/17/2000 |
| SEQ ID NO:3122 | 60/190,479 | 03/17/2000 |
| SEQ ID NO:3123 | 60/190,479 | 03/17/2000 |
| SEQ ID NO:3124 | 60/190,479 | 03/17/2000 |
| SEQ ID NO:3125 | 60/190,479 | 03/17/2000 |
| SEQ ID NO:3126 | 60/190,479 | 03/17/2000 |
| SEQ ID NO:3127 | 60/190,479 | 03/17/2000 |
| SEQ ID NO:3128 | 60/190,479 | 03/17/2000 |
| SEQ ID NO:3129 | 60/190,479 | 03/17/2000 |
| SEQ ID NO:3130 | 60/190,479 | 03/17/2000 |
| SEQ ID NO:3131 | 60/190,479 | 03/17/2000 |
| SEQ ID NO:3132 | 60/190,479 | 03/17/2000 |
| SEQ ID NO:3133 | 60/190,479 | 03/17/2000 |
| SEQ ID NO:3134 | 60/190,479 | 03/17/2000 |
| SEQ ID NO:3135 | 60/190,479 | 03/17/2000 |
| SEQ ID NO:3136 | 60/190,479 | 03/17/2000 |
| SEQ ID NO:3137 | 60/190,479 | 03/17/2000 |
| SEQ ID NO:3138 | 60/190,479 | 03/17/2000 |
| SEQ ID NO:3139 | 60/190,479 | 03/17/2000 |
| SEQ ID NO:3140 | 60/190,479 | 03/17/2000 |
| SEQ ID NO:3141 | 60/190,479 | 03/17/2000 |
| SEQ ID NO:3142 | 60/190,479 | 03/17/2000 |
| SEQ ID NO:3143 | 60/190,479 | 03/17/2000 |
| SEQ ID NO:3144 | 60/190,479 | 03/17/2000 |
| SEQ ID NO:3145 | 60/190,479 | 03/17/2000 |
| SEQ ID NO:3146 | 60/190,479 | 03/17/2000 |
| SEQ ID NO:3147 | 60/190,479 | 03/17/2000 |
| SEQ ID NO:3148 | 60/190,479 | 03/17/2000 |
| SEQ ID NO:3149 | 60/190,479 | 03/17/2000 |
| SEQ ID NO:3150 | 60/190,479 | 03/17/2000 |
| SEQ ID NO:3151 | 60/190,479 | 03/17/2000 |
| SEQ ID NO:3152 | 60/190,479 | 03/17/2000 |
| SEQ ID NO:3153 | 60/190,479 | 03/17/2000 |
| SEQ ID NO:3154 | 60/190,479 | 03/17/2000 |
| SEQ ID NO:3155 | 60/190,479 | 03/17/2000 |
| SEQ ID NO:3156 | 60/190,479 | 03/17/2000 |
| SEQ ID NO:3157 | 60/190,479 | 03/17/2000 |
| SEQ ID NO:3158 | 60/190,479 | 03/17/2000 |
| SEQ ID NO:3159 | 60/190,479 | 03/17/2000 |
| SEQ ID NO:3160 | 60/190,479 | 03/17/2000 |
| SEQ ID NO:3161 | 60/190,479 | 03/17/2000 |
| SEQ ID NO:3162 | 60/190,479 | 03/17/2000 |
| SEQ ID NO:3163 | 60/190,479 | 03/17/2000 |
| SEQ ID NO:3164 | 60/190,479 | 03/17/2000 |
| SEQ ID NO:3165 | 60/190,479 | 03/17/2000 |
| SEQ ID NO:3166 | 60/190,479 | 03/17/2000 |
| SEQ ID NO:3167 | 60/190,479 | 03/17/2000 |
| SEQ ID NO:3168 | 60/190,479 | 03/17/2000 |
| SEQ ID NO:3169 | 60/190,479 | 03/17/2000 |
| SEQ ID NO:3170 | 60/190,479 | 03/17/2000 |
| SEQ ID NO:3171 | 60/190,479 | 03/17/2000 |
| SEQ ID NO:3172 | 60/190,479 | 03/17/2000 |
| SEQ ID NO:3173 | 60/190,479 | 03/17/2000 |
| SEQ ID NO:3174 | 60/190,479 | 03/17/2000 |
| SEQ ID NO:3175 | 60/190,479 | 03/17/2000 |
| SEQ ID NO:3176 | 60/190,479 | 03/17/2000 |
| SEQ ID NO:3177 | 60/190,479 | 03/17/2000 |
| SEQ ID NO:3178 | 60/190,479 | 03/17/2000 |
| SEQ ID NO:3179 | 60/190,479 | 03/17/2000 |
| SEQ ID NO:3180 | 60/190,479 | 03/17/2000 |
| SEQ ID NO:3181 | 60/190,479 | 03/17/2000 |
| SEQ ID NO:3182 | 60/190,479 | 03/17/2000 |
| SEQ ID NO:3183 | 60/190,479 | 03/17/2000 |
| SEQ ID NO:3184 | 60/190,479 | 03/17/2000 |
| SEQ ID NO:3185 | 60/190,479 | 03/17/2000 |
| SEQ ID NO:3186 | 60/190,479 | 03/17/2000 |
| SEQ ID NO:3187 | 60/190,479 | 03/17/2000 |
| SEQ ID NO:3188 | 60/190,479 | 03/17/2000 |
| SEQ ID NO:3189 | 60/190,479 | 03/17/2000 |
| SEQ ID NO:3190 | 60/190,479 | 03/17/2000 |
| SEQ ID NO:3191 | 60/190,479 | 03/17/2000 |
| SEQ ID NO:3192 | 60/190,479 | 03/17/2000 |
| SEQ ID NO:3193 | 60/190,479 | 03/17/2000 |
| SEQ ID NO:3194 | 60/190,479 | 03/17/2000 |
| SEQ ID NO:3195 | 60/190,479 | 03/17/2000 |
| SEQ ID NO:3196 | 60/190,479 | 03/17/2000 |
| SEQ ID NO:3197 | 60/190,479 | 03/17/2000 |
| SEQ ID NO:3198 | 60/190,479 | 03/17/2000 |
| SEQ ID NO:3199 | 60/190,479 | 03/17/2000 |
| SEQ ID NO:3200 | 60/190,479 | 03/17/2000 |
| SEQ ID NO:3201 | 60/190,479 | 03/17/2000 |
| SEQ ID NO:3202 | 60/190,479 | 03/17/2000 |
| SEQ ID NO:3203 | 60/190,479 | 03/17/2000 |
| SEQ ID NO:3204 | 60/190,479 | 03/17/2000 |
| SEQ ID NO:3205 | 60/190,479 | 03/17/2000 |
| SEQ ID NO:3206 | 60/190,479 | 03/17/2000 |
| SEQ ID NO:3207 | 60/190,479 | 03/17/2000 |
| SEQ ID NO:3208 | 60/190,479 | 03/17/2000 |
| SEQ ID NO:3209 | 60/190,479 | 03/17/2000 |
| SEQ ID NO:3210 | 60/190,479 | 03/17/2000 |
| SEQ ID NO:3211 | 60/190,479 | 03/17/2000 |
| SEQ ID NO:3212 | 60/190,479 | 03/17/2000 |
| SEQ ID NO:3213 | 60/190,479 | 03/17/2000 |
| SEQ ID NO:3214 | 60/190,479 | 03/17/2000 |
| SEQ ID NO:3215 | 60/190,479 | 03/17/2000 |
| SEQ ID NO:3216 | 60/190,479 | 03/17/2000 |
| SEQ ID NO:3217 | 60/190,479 | 03/17/2000 |
| SEQ ID NO:3218 | 60/190,479 | 03/17/2000 |
| SEQ ID NO:3219 | 60/190,479 | 03/17/2000 |
| SEQ ID NO:3220 | 60/190,479 | 03/17/2000 |
| SEQ ID NO:3221 | 60/190,479 | 03/17/2000 |
| SEQ ID NO:3222 | 60/190,479 | 03/17/2000 |
| SEQ ID NO:3223 | 60/190,479 | 03/17/2000 |
| SEQ ID NO:3224 | 60/190,479 | 03/17/2000 |
| SEQ ID NO:3225 | 60/190,479 | 03/17/2000 |
| SEQ ID NO:3226 | 60/190,479 | 03/17/2000 |
| SEQ ID NO:3227 | 60/190,479 | 03/17/2000 |
| SEQ ID NO:3228 | 60/190,479 | 03/17/2000 |
| SEQ ID NO:3229 | 60/190,479 | 03/17/2000 |
| SEQ ID NO:3230 | 60/190,479 | 03/17/2000 |
| SEQ ID NO:3231 | 60/190,479 | 03/17/2000 |
| SEQ ID NO:3232 | 60/190,479 | 03/17/2000 |
| SEQ ID NO:3233 | 60/190,479 | 03/17/2000 |
| SEQ ID NO:3234 | 60/190,479 | 03/17/2000 |
| SEQ ID NO:3235 | 60/190,479 | 03/17/2000 |
| SEQ ID NO:3236 | 60/190,479 | 03/17/2000 |
| SEQ ID NO:3237 | 60/190,479 | 03/17/2000 |
| SEQ ID NO:3238 | 60/190,479 | 03/17/2000 |
| SEQ ID NO:3239 | 60/190,479 | 03/17/2000 |
| SEQ ID NO:3240 | 60/190,479 | 03/17/2000 |
| SEQ ID NO:3241 | 60/190,479 | 03/17/2000 |
| SEQ ID NO:3242 | 60/190,479 | 03/17/2000 |
| SEQ ID NO:3243 | 60/190,479 | 03/17/2000 |
| SEQ ID NO:3244 | 60/190,479 | 03/17/2000 |
| SEQ ID NO:3245 | 60/190,479 | 03/17/2000 |
| SEQ ID NO:3246 | 60/190,479 | 03/17/2000 |
| SEQ ID NO:3247 | 60/190,479 | 03/17/2000 |
| SEQ ID NO:3248 | 60/190,479 | 03/17/2000 |
| SEQ ID NO:3249 | 60/190,479 | 03/17/2000 |
| SEQ ID NO:3250 | 60/190,479 | 03/17/2000 |
| SEQ ID NO:3251 | 60/190,479 | 03/17/2000 |
| SEQ ID NO:3252 | 60/190,479 | 03/17/2000 |
| SEQ ID NO:3253 | 60/190,479 | 03/17/2000 |
| SEQ ID NO:3254 | 60/190,479 | 03/17/2000 |
| SEQ ID NO:3255 | 60/190,479 | 03/17/2000 |
| SEQ ID NO:3256 | 60/190,479 | 03/17/2000 |
| SEQ ID NO:3257 | 60/190,479 | 03/17/2000 |
| SEQ ID NO:3258 | 60/190,479 | 03/17/2000 |
| SEQ ID NO:3259 | 60/190,479 | 03/17/2000 |
| SEQ ID NO:3260 | 60/190,479 | 03/17/2000 |
| SEQ ID NO:3261 | 60/190,479 | 03/17/2000 |
| SEQ ID NO:3262 | 60/190,479 | 03/17/2000 |
| SEQ ID NO:3263 | 60/190,479 | 03/17/2000 |
| SEQ ID NO:3264 | 60/190,479 | 03/17/2000 |
| SEQ ID NO:3265 | 60/190,479 | 03/17/2000 |
| SEQ ID NO:3266 | 60/190,479 | 03/17/2000 |
| SEQ ID NO:3267 | 60/190,479 | 03/17/2000 |
| SEQ ID NO:3268 | 60/190,479 | 03/17/2000 |
| SEQ ID NO:3269 | 60/190,479 | 03/17/2000 |
| SEQ ID NO:3270 | 60/190,479 | 03/17/2000 |
| SEQ ID NO:3271 | 60/190,479 | 03/17/2000 |
| SEQ ID NO:3272 | 60/190,479 | 03/17/2000 |
| SEQ ID NO:3273 | 60/190,479 | 03/17/2000 |
| SEQ ID NO:3274 | 60/190,479 | 03/17/2000 |
| SEQ ID NO:3275 | 60/190,479 | 03/17/2000 |
| SEQ ID NO:3276 | 60/190,479 | 03/17/2000 |
| SEQ ID NO:3277 | 60/190,479 | 03/17/2000 |
| SEQ ID NO:3278 | 60/190,479 | 03/17/2000 |
| SEQ ID NO:3279 | 60/190,479 | 03/17/2000 |
| SEQ ID NO:3280 | 60/190,479 | 03/17/2000 |
| SEQ ID NO:3281 | 60/190,479 | 03/17/2000 |
| SEQ ID NO:3282 | 60/190,479 | 03/17/2000 |
| SEQ ID NO:3283 | 60/190,479 | 03/17/2000 |
| SEQ ID NO:3284 | 60/190,479 | 03/17/2000 |
| SEQ ID NO:3285 | 60/190,479 | 03/17/2000 |
| SEQ ID NO:3286 | 60/190,479 | 03/17/2000 |
| SEQ ID NO:3287 | 60/190,479 | 03/17/2000 |
| SEQ ID NO:3288 | 60/190,479 | 03/17/2000 |
| SEQ ID NO:3289 | 60/190,479 | 03/17/2000 |
| SEQ ID NO:3290 | 60/190,479 | 03/17/2000 |
| SEQ ID NO:3291 | 60/190,479 | 03/17/2000 |
| SEQ ID NO:3292 | 60/190,479 | 03/17/2000 |
| SEQ ID NO:3293 | 60/190,479 | 03/17/2000 |
| SEQ ID NO:3294 | 60/190,479 | 03/17/2000 |
| SEQ ID NO:3295 | 60/190,479 | 03/17/2000 |
| SEQ ID NO:3296 | 60/190,479 | 03/17/2000 |
| SEQ ID NO:3297 | 60/190,479 | 03/17/2000 |
| SEQ ID NO:3298 | 60/190,479 | 03/17/2000 |
| SEQ ID NO:3299 | 60/190,479 | 03/17/2000 |
| SEQ ID NO:3300 | 60/190,479 | 03/17/2000 |
| SEQ ID NO:3301 | 60/190,479 | 03/17/2000 |
| SEQ ID NO:3302 | 60/190,479 | 03/17/2000 |
| SEQ ID NO:3303 | 60/190,479 | 03/17/2000 |
| SEQ ID NO:3304 | 60/190,479 | 03/17/2000 |
| SEQ ID NO:3305 | 60/190,479 | 03/17/2000 |
| SEQ ID NO:3306 | 60/190,479 | 03/17/2000 |
| SEQ ID NO:3307 | 60/190,479 | 03/17/2000 |
| SEQ ID NO:3308 | 60/190,479 | 03/17/2000 |
| SEQ ID NO:3309 | 60/190,479 | 03/17/2000 |
| SEQ ID NO:3310 | 60/190,479 | 03/17/2000 |
| SEQ ID NO:3311 | 60/190,479 | 03/17/2000 |
| SEQ ID NO:3312 | 60/190,479 | 03/17/2000 |
| SEQ ID NO:3313 | 60/190,479 | 03/17/2000 |
| SEQ ID NO:3314 | 60/190,479 | 03/17/2000 |
| SEQ ID NO:3315 | 60/190,479 | 03/17/2000 |
| SEQ ID NO:3316 | 60/190,479 | 03/17/2000 |
| SEQ ID NO:3317 | 60/190,479 | 03/17/2000 |
| SEQ ID NO:3318 | 60/190,479 | 03/17/2000 |
| SEQ ID NO:3319 | 60/190,479 | 03/17/2000 |
| SEQ ID NO:3320 | 60/190,479 | 03/17/2000 |
| SEQ ID NO:3321 | 60/190,479 | 03/17/2000 |
| SEQ ID NO:3322 | 60/190,479 | 03/17/2000 |
| SEQ ID NO:3323 | 60/190,479 | 03/17/2000 |
| SEQ ID NO:3324 | 60/190,479 | 03/17/2000 |
| SEQ ID NO:3325 | 60/190,479 | 03/17/2000 |
| SEQ ID NO:3326 | 60/190,479 | 03/17/2000 |
| SEQ ID NO:3327 | 60/190,479 | 03/17/2000 |
| SEQ ID NO:3328 | 60/190,479 | 03/17/2000 |
| SEQ ID NO:3329 | 60/190,479 | 03/17/2000 |
| SEQ ID NO:3330 | 60/190,479 | 03/17/2000 |
| SEQ ID NO:3331 | 60/190,479 | 03/17/2000 |
| SEQ ID NO:3332 | 60/190,479 | 03/17/2000 |
| SEQ ID NO:3333 | 60/190,479 | 03/17/2000 |
| SEQ ID NO:3334 | 60/190,479 | 03/17/2000 |
| SEQ ID NO:3335 | 60/190,479 | 03/17/2000 |
| SEQ ID NO:3336 | 60/190,479 | 03/17/2000 |
| SEQ ID NO:3337 | 60/190,479 | 03/17/2000 |
| SEQ ID NO:3338 | 60/190,479 | 03/17/2000 |
| SEQ ID NO:3339 | 60/190,479 | 03/17/2000 |
| SEQ ID NO:3340 | 60/190,479 | 03/17/2000 |
| SEQ ID NO:3341 | 60/190,479 | 03/17/2000 |
| SEQ ID NO:3342 | 60/190,479 | 03/17/2000 |
| SEQ ID NO:3343 | 60/190,479 | 03/17/2000 |
| SEQ ID NO:3344 | 60/190,479 | 03/17/2000 |
| SEQ ID NO:3345 | 60/190,479 | 03/17/2000 |
| SEQ ID NO:3346 | 60/190,479 | 03/17/2000 |
| SEQ ID NO:3347 | 60/190,479 | 03/17/2000 |
| SEQ ID NO:3348 | 60/190,479 | 03/17/2000 |
| SEQ ID NO:3349 | 60/190,479 | 03/17/2000 |
| SEQ ID NO:3350 | 60/190,479 | 03/17/2000 |
| SEQ ID NO:3351 | 60/190,479 | 03/17/2000 |
| SEQ ID NO:3352 | 60/190,479 | 03/17/2000 |
| SEQ ID NO:3353 | 60/190,479 | 03/17/2000 |
| SEQ ID NO:3354 | 60/190,479 | 03/17/2000 |
| SEQ ID NO:3355 | 60/190,479 | 03/17/2000 |
| SEQ ID NO:3356 | 60/190,479 | 03/17/2000 |
| SEQ ID NO:3357 | 60/190,479 | 03/17/2000 |
| SEQ ID NO:3358 | 60/190,479 | 03/17/2000 |
| SEQ ID NO:3359 | 60/190,479 | 03/17/2000 |
| SEQ ID NO:3360 | 60/190,479 | 03/17/2000 |
| SEQ ID NO:3361 | 60/190,479 | 03/17/2000 |
| SEQ ID NO:3362 | 60/190,479 | 03/17/2000 |
| SEQ ID NO:3363 | 60/190,479 | 03/17/2000 |
| SEQ ID NO:3364 | 60/190,479 | 03/17/2000 |
| SEQ ID NO:3365 | 60/190,479 | 03/17/2000 |
| SEQ ID NO:3366 | 60/190,479 | 03/17/2000 |
| SEQ ID NO:3367 | 60/190,479 | 03/17/2000 |
| SEQ ID NO:3368 | 60/190,479 | 03/17/2000 |
| SEQ ID NO:3369 | 60/190,479 | 03/17/2000 |
| SEQ ID NO:3370 | 60/190,479 | 03/17/2000 |
| SEQ ID NO:3371 | 60/190,479 | 03/17/2000 |
| SEQ ID NO:3372 | 60/190,479 | 03/17/2000 |
| SEQ ID NO:3373 | 60/190,479 | 03/17/2000 |
| SEQ ID NO:3374 | 60/190,479 | 03/17/2000 |
| SEQ ID NO:3375 | 60/190,479 | 03/17/2000 |
| SEQ ID NO:3376 | 60/190,479 | 03/17/2000 |
| SEQ ID NO:3377 | 60/190,479 | 03/17/2000 |
| SEQ ID NO:3378 | 60/190,479 | 03/17/2000 |
| SEQ ID NO:3379 | 60/190,479 | 03/17/2000 |
| SEQ ID NO:3380 | 60/190,479 | 03/17/2000 |
| SEQ ID NO:3381 | 60/190,479 | 03/17/2000 |
| SEQ ID NO:3382 | 60/190,479 | 03/17/2000 |
| SEQ ID NO:3383 | 60/190,479 | 03/17/2000 |
| SEQ ID NO:3384 | 60/190,479 | 03/17/2000 |
| SEQ ID NO:3385 | 60/190,479 | 03/17/2000 |
| SEQ ID NO:3386 | 60/190,479 | 03/17/2000 |
| SEQ ID NO:3387 | 60/190,479 | 03/17/2000 |
| SEQ ID NO:3388 | 60/190,479 | 03/17/2000 |
| SEQ ID NO:3389 | 60/190,479 | 03/17/2000 |
| SEQ ID NO:3390 | 60/190,479 | 03/17/2000 |
| SEQ ID NO:3391 | 60/190,479 | 03/17/2000 |
| SEQ ID NO:3392 | 60/190,479 | 03/17/2000 |
| SEQ ID NO:3393 | 60/190,479 | 03/17/2000 |
| SEQ ID NO:3394 | 60/190,479 | 03/17/2000 |
| SEQ ID NO:3395 | 60/190,479 | 03/17/2000 |
| SEQ ID NO:3396 | 60/190,479 | 03/17/2000 |
| SEQ ID NO:3397 | 60/190,479 | 03/17/2000 |
| SEQ ID NO:3398 | 60/190,479 | 03/17/2000 |
| SEQ ID NO:3399 | 60/190,479 | 03/17/2000 |
| SEQ ID NO:3400 | 60/190,479 | 03/17/2000 |
| SEQ ID NO:3401 | 60/190,479 | 03/17/2000 |
| SEQ ID NO:3402 | 60/190,479 | 03/17/2000 |
| SEQ ID NO:3403 | 60/190,479 | 03/17/2000 |
| SEQ ID NO:3404 | 60/190,479 | 03/17/2000 |
| SEQ ID NO:3405 | 60/190,479 | 03/17/2000 |
| SEQ ID NO:3406 | 60/190,479 | 03/17/2000 |
| SEQ ID NO:3407 | 60/190,479 | 03/17/2000 |
| SEQ ID NO:3408 | 60/190,479 | 03/17/2000 |
| SEQ ID NO:3409 | 60/190,479 | 03/17/2000 |
| SEQ ID NO:3410 | 60/190,479 | 03/17/2000 |
| SEQ ID NO:3411 | 60/190,479 | 03/17/2000 |
| SEQ ID NO:3412 | 60/190,479 | 03/17/2000 |
| SEQ ID NO:3413 | 60/190,479 | 03/17/2000 |
| SEQ ID NO:3414 | 60/190,479 | 03/17/2000 |
| SEQ ID NO:3415 | 60/190,479 | 03/17/2000 |
| SEQ ID NO:3416 | 60/190,479 | 03/17/2000 |
| SEQ ID NO:3417 | 60/190,479 | 03/17/2000 |
| SEQ ID NO:3418 | 60/190,479 | 03/17/2000 |
| SEQ ID NO:3419 | 60/190,479 | 03/17/2000 |
| SEQ ID NO:3420 | 60/190,479 | 03/17/2000 |
| SEQ ID NO:3421 | 60/190,479 | 03/17/2000 |
| SEQ ID NO:3422 | 60/190,479 | 03/17/2000 |
| SEQ ID NO:3423 | 60/190,479 | 03/17/2000 |
| SEQ ID NO:3424 | 60/190,479 | 03/17/2000 |
| SEQ ID NO:3425 | 60/190,479 | 03/17/2000 |
| SEQ ID NO:3426 | 60/190,479 | 03/17/2000 |
| SEQ ID NO:3427 | 60/190,479 | 03/17/2000 |
| SEQ ID NO:3428 | 60/190,479 | 03/17/2000 |
| SEQ ID NO:3429 | 60/190,479 | 03/17/2000 |
| SEQ ID NO:3430 | 60/190,479 | 03/17/2000 |
| SEQ ID NO:3431 | 60/190,479 | 03/17/2000 |
| SEQ ID NO:3432 | 60/190,479 | 03/17/2000 |
| SEQ ID NO:3433 | 60/190,479 | 03/17/2000 |
| SEQ ID NO:3434 | 60/190,479 | 03/17/2000 |
| SEQ ID NO:3435 | 60/190,479 | 03/17/2000 |
| SEQ ID NO:3436 | 60/190,479 | 03/17/2000 |
| SEQ ID NO:3437 | 60/190,479 | 03/17/2000 |
| SEQ ID NO:3438 | 60/190,479 | 03/17/2000 |
| SEQ ID NO:3439 | 60/190,479 | 03/17/2000 |
| SEQ ID NO:3440 | 60/190,479 | 03/17/2000 |
| SEQ ID NO:3441 | 60/190,479 | 03/17/2000 |
| SEQ ID NO:3442 | 60/190,479 | 03/17/2000 |
| SEQ ID NO:3443 | 60/190,479 | 03/17/2000 |
| SEQ ID NO:3444 | 60/190,479 | 03/17/2000 |
| SEQ ID NO:3445 | 60/190,479 | 03/17/2000 |
| SEQ ID NO:3446 | 60/190,479 | 03/17/2000 |
| SEQ ID NO:3447 | 60/190,479 | 03/17/2000 |
| SEQ ID NO:3448 | 60/190,479 | 03/17/2000 |
| SEQ ID NO:3449 | 60/190,479 | 03/17/2000 |
| SEQ ID NO:3450 | 60/190,479 | 03/17/2000 |
| SEQ ID NO:3451 | 60/190,479 | 03/17/2000 |
| SEQ ID NO:3452 | 60/190,479 | 03/17/2000 |
| SEQ ID NO:3453 | 60/190,479 | 03/17/2000 |
| SEQ ID NO:3454 | 60/190,479 | 03/17/2000 |
| SEQ ID NO:3455 | 60/190,479 | 03/17/2000 |
| SEQ ID NO:3456 | 60/190,479 | 03/17/2000 |
| SEQ ID NO:3457 | 60/190,479 | 03/17/2000 |
| SEQ ID NO:3458 | 60/190,479 | 03/17/2000 |
| SEQ ID NO:3459 | 60/190,479 | 03/17/2000 |
| SEQ ID NO:3460 | 60/190,479 | 03/17/2000 |
| SEQ ID NO:3461 | 60/190,479 | 03/17/2000 |
| SEQ ID NO:3462 | 60/190,479 | 03/17/2000 |
| SEQ ID NO:3463 | 60/190,479 | 03/17/2000 |
| SEQ ID NO:3464 | 60/190,479 | 03/17/2000 |
| SEQ ID NO:3465 | 60/190,479 | 03/17/2000 |
| SEQ ID NO:3466 | 60/190,479 | 03/17/2000 |
| SEQ ID NO:3467 | 60/190,479 | 03/17/2000 |
| SEQ ID NO:3468 | 60/190,479 | 03/17/2000 |
| SEQ ID NO:3469 | 60/190,479 | 03/17/2000 |
| SEQ ID NO:3470 | 60/190,479 | 03/17/2000 |
| SEQ ID NO:3471 | 60/190,479 | 03/17/2000 |
| SEQ ID NO:3472 | 60/190,479 | 03/17/2000 |
| SEQ ID NO:3473 | 60/190,479 | 03/17/2000 |
| SEQ ID NO:3474 | 60/190,479 | 03/17/2000 |
| SEQ ID NO:3475 | 60/190,479 | 03/17/2000 |
| SEQ ID NO:3476 | 60/190,479 | 03/17/2000 |
| SEQ ID NO:3477 | 60/190,479 | 03/17/2000 |
| SEQ ID NO:3478 | 60/190,479 | 03/17/2000 |
| SEQ ID NO:3479 | 60/190,479 | 03/17/2000 |
| SEQ ID NO:3480 | 60/190,479 | 03/17/2000 |
| SEQ ID NO:3481 | 60/190,479 | 03/17/2000 |
| SEQ ID NO:3482 | 60/190,479 | 03/17/2000 |
| SEQ ID NO:3483 | 60/190,479 | 03/17/2000 |
| SEQ ID NO:3484 | 60/190,479 | 03/17/2000 |
| SEQ ID NO:3485 | 60/190,479 | 03/17/2000 |
| SEQ ID NO:3486 | 60/190,479 | 03/17/2000 |
| SEQ ID NO:3487 | 60/190,479 | 03/17/2000 |
| SEQ ID NO:3488 | 60/190,479 | 03/17/2000 |
| SEQ ID NO:3489 | 60/190,479 | 03/17/2000 |
| SEQ ID NO:3490 | 60/190,479 | 03/17/2000 |
| SEQ ID NO:3491 | 60/190,479 | 03/17/2000 |
| SEQ ID NO:3492 | 60/190,479 | 03/17/2000 |
| SEQ ID NO:3493 | 60/190,479 | 03/17/2000 |
| SEQ ID NO:3494 | 60/190,479 | 03/17/2000 |
| SEQ ID NO:3495 | 60/190,479 | 03/17/2000 |
| SEQ ID NO:3496 | 60/190,479 | 03/17/2000 |
| SEQ ID NO:3497 | 60/190,479 | 03/17/2000 |
| SEQ ID NO:3498 | 60/190,479 | 03/17/2000 |
| SEQ ID NO:3499 | 60/190,479 | 03/17/2000 |
| SEQ ID NO:3500 | 60/190,479 | 03/17/2000 |
| SEQ ID NO:3501 | 60/190,479 | 03/17/2000 |
| SEQ ID NO:3502 | 60/190,479 | 03/17/2000 |
| SEQ ID NO:3503 | 60/190,479 | 03/17/2000 |
| SEQ ID NO:3504 | 60/190,479 | 03/17/2000 |
| SEQ ID NO:3505 | 60/190,479 | 03/17/2000 |
| SEQ ID NO:3506 | 60/190,479 | 03/17/2000 |
| SEQ ID NO:3507 | 60/190,479 | 03/17/2000 |
| SEQ ID NO:3508 | 60/190,479 | 03/17/2000 |
| SEQ ID NO:3509 | 60/190,479 | 03/17/2000 |
| SEQ ID NO:3510 | 60/190,479 | 03/17/2000 |
| SEQ ID NO:3511 | 60/190,479 | 03/17/2000 |
| SEQ ID NO:3512 | 60/190,479 | 03/17/2000 |
| SEQ ID NO:3513 | 60/190,479 | 03/17/2000 |
| SEQ ID NO:3514 | 60/190,479 | 03/17/2000 |
| SEQ ID NO:3515 | 60/190,479 | 03/17/2000 |
| SEQ ID NO:3516 | 60/190,479 | 03/17/2000 |
| SEQ ID NO:3517 | 60/190,479 | 03/17/2000 |
| SEQ ID NO:3518 | 60/190,479 | 03/17/2000 |
| SEQ ID NO:3519 | 60/190,479 | 03/17/2000 |
| SEQ ID NO:3520 | 60/190,479 | 03/17/2000 |
| SEQ ID NO:3521 | 60/190,479 | 03/17/2000 |
| SEQ ID NO:3522 | 60/190,479 | 03/17/2000 |
| SEQ ID NO:3523 | 60/190,479 | 03/17/2000 |
| SEQ ID NO:3524 | 60/190,479 | 03/17/2000 |
| SEQ ID NO:3525 | 60/190,479 | 03/17/2000 |
| SEQ ID NO:3526 | 60/190,479 | 03/17/2000 |
| SEQ ID NO:3527 | 60/190,479 | 03/17/2000 |
| SEQ ID NO:3528 | 60/190,479 | 03/17/2000 |
| SEQ ID NO:3529 | 60/190,479 | 03/17/2000 |
| SEQ ID NO:3530 | 60/190,479 | 03/17/2000 |
| SEQ ID NO:3531 | 60/190,479 | 03/17/2000 |
| SEQ ID NO:3532 | 60/190,479 | 03/17/2000 |
| SEQ ID NO:3533 | 60/190,479 | 03/17/2000 |
| SEQ ID NO:3534 | 60/190,479 | 03/17/2000 |
| SEQ ID NO:3535 | 60/190,479 | 03/17/2000 |
| SEQ ID NO:3536 | 60/190,479 | 03/17/2000 |
| SEQ ID NO:3537 | 60/190,479 | 03/17/2000 |
| SEQ ID NO:3538 | 60/190,479 | 03/17/2000 |
| SEQ ID NO:3539 | 60/190,479 | 03/17/2000 |
| SEQ ID NO:3540 | 60/190,479 | 03/17/2000 |
| SEQ ID NO:3541 | 60/190,479 | 03/17/2000 |
| SEQ ID NO:3542 | 60/190,479 | 03/17/2000 |
| SEQ ID NO:3543 | 60/190,479 | 03/17/2000 |
| SEQ ID NO:3544 | 60/190,479 | 03/17/2000 |
| SEQ ID NO:3545 | 60/190,479 | 03/17/2000 |
| SEQ ID NO:3546 | 60/190,479 | 03/17/2000 |
| SEQ ID NO:3547 | 60/190,479 | 03/17/2000 |
| SEQ ID NO:3548 | 60/190,479 | 03/17/2000 |
| SEQ ID NO:3549 | 60/190,479 | 03/17/2000 |
| SEQ ID NO:3550 | 60/190,479 | 03/17/2000 |
| SEQ ID NO:3551 | 60/190,479 | 03/17/2000 |
| SEQ ID NO:3552 | 60/190,479 | 03/17/2000 |
| SEQ ID NO:3553 | 60/190,479 | 03/17/2000 |
| SEQ ID NO:3554 | 60/190,479 | 03/17/2000 |
| SEQ ID NO:3555 | 60/190,479 | 03/17/2000 |
| SEQ ID NO:3556 | 60/190,479 | 03/17/2000 |
| SEQ ID NO:3557 | 60/190,479 | 03/17/2000 |
| SEQ ID NO:3558 | 60/190,479 | 03/17/2000 |
| SEQ ID NO:3559 | 60/190,479 | 03/17/2000 |
| SEQ ID NO:3560 | 60/190,479 | 03/17/2000 |
| SEQ ID NO:3561 | 60/190,479 | 03/17/2000 |
| SEQ ID NO:3562 | 60/190,479 | 03/17/2000 |
| SEQ ID NO:3563 | 60/190,479 | 03/17/2000 |
| SEQ ID NO:3564 | 60/190,479 | 03/17/2000 |
| SEQ ID NO:3565 | 60/190,479 | 03/17/2000 |
| SEQ ID NO:3566 | 60/190,479 | 03/17/2000 |
| SEQ ID NO:3567 | 60/190,479 | 03/17/2000 |
| SEQ ID NO:3568 | 60/190,479 | 03/17/2000 |
| SEQ ID NO:3569 | 60/190,479 | 03/17/2000 |
| SEQ ID NO:3570 | 60/190,479 | 03/17/2000 |
| SEQ ID NO:3571 | 60/190,479 | 03/17/2000 |
| SEQ ID NO:3572 | 60/190,479 | 03/17/2000 |
| SEQ ID NO:3573 | 60/190,479 | 03/17/2000 |
| SEQ ID NO:3574 | 60/190,479 | 03/17/2000 |
| SEQ ID NO:3575 | 60/190,479 | 03/17/2000 |
| SEQ ID NO:3576 | 60/190,479 | 03/17/2000 |
| SEQ ID NO:3577 | 60/190,479 | 03/17/2000 |
| SEQ ID NO:3578 | 60/190,479 | 03/17/2000 |
| SEQ ID NO:3579 | 60/190,479 | 03/17/2000 |
| SEQ ID NO:3580 | 60/190,479 | 03/17/2000 |
| SEQ ID NO:3581 | 60/190,479 | 03/17/2000 |
| SEQ ID NO:3582 | 60/190,479 | 03/17/2000 |
| SEQ ID NO:3583 | 60/190,479 | 03/17/2000 |
| SEQ ID NO:3584 | 60/190,479 | 03/17/2000 |
| SEQ ID NO:3585 | 60/190,479 | 03/17/2000 |
| SEQ ID NO:3586 | 60/190,479 | 03/17/2000 |
| SEQ ID NO:3587 | 60/190,479 | 03/17/2000 |
| SEQ ID NO:3588 | 60/190,479 | 03/17/2000 |
| SEQ ID NO:3589 | 60/190,479 | 03/17/2000 |
| SEQ ID NO:3590 | 60/190,479 | 03/17/2000 |
| SEQ ID NO:3591 | 60/190,479 | 03/17/2000 |
| SEQ ID NO:3592 | 60/190,479 | 03/17/2000 |
| SEQ ID NO:3593 | 60/190,479 | 03/17/2000 |
| SEQ ID NO:3594 | 60/190,479 | 03/17/2000 |
| SEQ ID NO:3595 | 60/190,479 | 03/17/2000 |
| SEQ ID NO:3596 | 60/190,479 | 03/17/2000 |
| SEQ ID NO:3597 | 60/190,479 | 03/17/2000 |
| SEQ ID NO:3598 | 60/190,479 | 03/17/2000 |
| SEQ ID NO:3599 | 60/190,479 | 03/17/2000 |
| SEQ ID NO:3600 | 60/190,479 | 03/17/2000 |
| SEQ ID NO:3601 | 60/190,479 | 03/17/2000 |
| SEQ ID NO:3602 | 60/190,479 | 03/17/2000 |
| SEQ ID NO:3603 | 60/190,479 | 03/17/2000 |
| SEQ ID NO:3604 | 60/190,479 | 03/17/2000 |
| SEQ ID NO:3605 | 60/190,479 | 03/17/2000 |
| SEQ ID NO:3606 | 60/190,479 | 03/17/2000 |
| SEQ ID NO:3607 | 60/190,479 | 03/17/2000 |
| SEQ ID NO:3608 | 60/190,479 | 03/17/2000 |
| SEQ ID NO:3609 | 60/190,479 | 03/17/2000 |
| SEQ ID NO:3610 | 60/190,479 | 03/17/2000 |
| SEQ ID NO:3611 | 60/190,479 | 03/17/2000 |
| SEQ ID NO:3612 | 60/190,479 | 03/17/2000 |
| SEQ ID NO:3613 | 60/190,479 | 03/17/2000 |
| SEQ ID NO:3614 | 60/190,479 | 03/17/2000 |
| SEQ ID NO:3615 | 60/190,479 | 03/17/2000 |
| SEQ ID NO:3616 | 60/190,479 | 03/17/2000 |
| SEQ ID NO:3617 | 60/190,479 | 03/17/2000 |
| SEQ ID NO:3618 | 60/190,479 | 03/17/2000 |
| SEQ ID NO:3619 | 60/190,479 | 03/17/2000 |
| SEQ ID NO:3620 | 60/190,479 | 03/17/2000 |
| SEQ ID NO:3621 | 60/190,479 | 03/17/2000 |
| SEQ ID NO:3622 | 60/190,479 | 03/17/2000 |
| SEQ ID NO:3623 | 60/190,479 | 03/17/2000 |
| SEQ ID NO:3624 | 60/190,479 | 03/17/2000 |
| SEQ ID NO:3625 | 60/190,479 | 03/17/2000 |
| SEQ ID NO:3626 | 60/190,479 | 03/17/2000 |
| SEQ ID NO:3627 | 60/190,479 | 03/17/2000 |
| SEQ ID NO:3628 | 60/190,479 | 03/17/2000 |
| SEQ ID NO:3629 | 60/190,479 | 03/17/2000 |
| SEQ ID NO:3630 | 60/190,479 | 03/17/2000 |
| SEQ ID NO:3631 | 60/190,479 | 03/17/2000 |
| SEQ ID NO:3632 | 60/190,479 | 03/17/2000 |
| SEQ ID NO:3633 | 60/190,479 | 03/17/2000 |
| SEQ ID NO:3634 | 60/190,479 | 03/17/2000 |
| SEQ ID NO:3635 | 60/190,479 | 03/17/2000 |
| SEQ ID NO:3636 | 60/190,479 | 03/17/2000 |
| SEQ ID NO:3637 | 60/190,479 | 03/17/2000 |
| SEQ ID NO:3638 | 60/190,479 | 03/17/2000 |
| SEQ ID NO:3639 | 60/190,479 | 03/17/2000 |
| SEQ ID NO:3640 | 60/190,479 | 03/17/2000 |
| SEQ ID NO:3641 | 60/190,479 | 03/17/2000 |
| SEQ ID NO:3642 | 60/190,479 | 03/17/2000 |
| SEQ ID NO:3643 | 60/190,479 | 03/17/2000 |
| SEQ ID NO:3644 | 60/190,479 | 03/17/2000 |
| SEQ ID NO:3645 | 60/190,479 | 03/17/2000 |
| SEQ ID NO:3646 | 60/190,479 | 03/17/2000 |
| SEQ ID NO:3647 | 60/190,479 | 03/17/2000 |
| SEQ ID NO:3648 | 60/190,479 | 03/17/2000 |
| SEQ ID NO:3649 | 60/190,479 | 03/17/2000 |
| SEQ ID NO:3650 | 60/190,479 | 03/17/2000 |
| SEQ ID NO:3651 | 60/190,479 | 03/17/2000 |
| SEQ ID NO:3652 | 60/190,479 | 03/17/2000 |
| SEQ ID NO:3653 | 60/190,479 | 03/17/2000 |
| SEQ ID NO:3654 | 60/190,479 | 03/17/2000 |
| SEQ ID NO:3655 | 60/190,479 | 03/17/2000 |
| SEQ ID NO:3656 | 60/190,479 | 03/17/2000 |
| SEQ ID NO:3657 | 60/190,479 | 03/17/2000 |
| SEQ ID NO:3658 | 60/190,479 | 03/17/2000 |
| SEQ ID NO:3659 | 60/190,479 | 03/17/2000 |
| SEQ ID NO:3660 | 60/190,479 | 03/17/2000 |
| SEQ ID NO:3661 | 60/190,479 | 03/17/2000 |
| SEQ ID NO:3662 | 60/190,479 | 03/17/2000 |
| SEQ ID NO:3663 | 60/190,479 | 03/17/2000 |
| SEQ ID NO:3664 | 60/190,479 | 03/17/2000 |
| SEQ ID NO:3665 | 60/190,479 | 03/17/2000 |
| SEQ ID NO:3666 | 60/190,479 | 03/17/2000 |
| SEQ ID NO:3667 | 60/190,479 | 03/17/2000 |
| SEQ ID NO:3668 | 60/190,479 | 03/17/2000 |
| SEQ ID NO:3669 | 60/190,479 | 03/17/2000 |
| SEQ ID NO:3670 | 60/190,479 | 03/17/2000 |
| SEQ ID NO:3671 | 60/190,479 | 03/17/2000 |
| SEQ ID NO:3672 | 60/190,479 | 03/17/2000 |
| SEQ ID NO:3673 | 60/190,479 | 03/17/2000 |
| SEQ ID NO:3674 | 60/190,479 | 03/17/2000 |
| SEQ ID NO:3675 | 60/190,479 | 03/17/2000 |
| SEQ ID NO:3676 | 60/190,479 | 03/17/2000 |
| SEQ ID NO:3677 | 60/190,479 | 03/17/2000 |
| SEQ ID NO:3678 | 60/190,479 | 03/17/2000 |
| SEQ ID NO:3679 | 60/190,479 | 03/17/2000 |
| SEQ ID NO:3680 | 60/190,479 | 03/17/2000 |
| SEQ ID NO:3681 | 60/190,479 | 03/17/2000 |
| SEQ ID NO:3682 | 60/190,479 | 03/17/2000 |
| SEQ ID NO:3683 | 60/190,479 | 03/17/2000 |
| SEQ ID NO:3684 | 60/190,479 | 03/17/2000 |
| SEQ ID NO:3685 | 60/190,479 | 03/17/2000 |
| SEQ ID NO:3686 | 60/190,479 | 03/17/2000 |
| SEQ ID NO:3687 | 60/190,479 | 03/17/2000 |
| SEQ ID NO:3688 | 60/190,479 | 03/17/2000 |
| SEQ ID NO:3689 | 60/190,479 | 03/17/2000 |
| SEQ ID NO:3690 | 60/190,479 | 03/17/2000 |
| SEQ ID NO:3691 | 60/190,479 | 03/17/2000 |
| SEQ ID NO:3692 | 60/190,479 | 03/17/2000 |
| SEQ ID NO:3693 | 60/190,479 | 03/17/2000 |
| SEQ ID NO:3694 | 60/190,479 | 03/17/2000 |
| SEQ ID NO:3695 | 60/190,479 | 03/17/2000 |
| SEQ ID NO:3696 | 60/190,479 | 03/17/2000 |
| SEQ ID NO:3697 | 60/190,479 | 03/17/2000 |
| SEQ ID NO:3698 | 60/190,479 | 03/17/2000 |
| SEQ ID NO:3699 | 60/190,479 | 03/17/2000 |
| SEQ ID NO:3700 | 60/190,479 | 03/17/2000 |
| SEQ ID NO:3701 | 60/190,479 | 03/17/2000 |
| SEQ ID NO:3702 | 60/190,479 | 03/17/2000 |
| SEQ ID NO:3703 | 60/190,479 | 03/17/2000 |
| SEQ ID NO:3704 | 60/190,479 | 03/17/2000 |
| SEQ ID NO:3705 | 60/190,479 | 03/17/2000 |
| SEQ ID NO:3706 | 60/190,479 | 03/17/2000 |
| SEQ ID NO:3707 | 60/190,479 | 03/17/2000 |
| SEQ ID NO:3708 | 60/190,479 | 03/17/2000 |
| SEQ ID NO:3709 | 60/190,479 | 03/17/2000 |
| SEQ ID NO:3710 | 60/190,479 | 03/17/2000 |
| SEQ ID NO:3711 | 60/190,479 | 03/17/2000 |
| SEQ ID NO:3712 | 60/190,479 | 03/17/2000 |
| SEQ ID NO:3713 | 60/190,479 | 03/17/2000 |
| SEQ ID NO:3714 | 60/190,479 | 03/17/2000 |
| SEQ ID NO:3715 | 60/190,479 | 03/17/2000 |
| SEQ ID NO:3716 | 60/190,479 | 03/17/2000 |
| SEQ ID NO:3717 | 60/190,479 | 03/17/2000 |
| SEQ ID NO:3718 | 60/190,479 | 03/17/2000 |
| SEQ ID NO:3719 | 60/190,479 | 03/17/2000 |
| SEQ ID NO:3720 | 60/190,479 | 03/17/2000 |
| SEQ ID NO:3721 | 60/190,479 | 03/17/2000 |
| SEQ ID NO:3722 | 60/190,479 | 03/17/2000 |
| SEQ ID NO:3723 | 60/190,479 | 03/17/2000 |
| SEQ ID NO:3724 | 60/190,479 | 03/17/2000 |
| SEQ ID NO:3725 | 60/190,479 | 03/17/2000 |
| SEQ ID NO:3726 | 60/190,479 | 03/17/2000 |
| SEQ ID NO:3727 | 60/190,479 | 03/17/2000 |
| SEQ ID NO:3728 | 60/190,479 | 03/17/2000 |
| SEQ ID NO:3729 | 60/190,479 | 03/17/2000 |
| SEQ ID NO:3730 | 60/190,479 | 03/17/2000 |
| SEQ ID NO:3731 | 60/190,479 | 03/17/2000 |
| SEQ ID NO:3732 | 60/190,479 | 03/17/2000 |
| SEQ ID NO:3733 | 60/190,479 | 03/17/2000 |
| SEQ ID NO:3734 | 60/190,479 | 03/17/2000 |
| SEQ ID NO:3735 | 60/190,479 | 03/17/2000 |
| SEQ ID NO:3736 | 60/190,479 | 03/17/2000 |
| SEQ ID NO:3737 | 60/190,479 | 03/17/2000 |
| SEQ ID NO:3738 | 60/190,479 | 03/17/2000 |
| SEQ ID NO:3739 | 60/190,479 | 03/17/2000 |
| SEQ ID NO:3740 | 60/190,479 | 03/17/2000 |
| SEQ ID NO:3741 | 60/190,479 | 03/17/2000 |
| SEQ ID NO:3742 | 60/190,479 | 03/17/2000 |
| SEQ ID NO:3743 | 60/190,479 | 03/17/2000 |
| SEQ ID NO:3744 | 60/190,479 | 03/17/2000 |
| SEQ ID NO:3745 | 60/190,479 | 03/17/2000 |
| SEQ ID NO:3746 | 60/190,479 | 03/17/2000 |
| SEQ ID NO:3747 | 60/190,479 | 03/17/2000 |
| SEQ ID NO:3748 | 60/190,479 | 03/17/2000 |
| SEQ ID NO:3749 | 60/190,479 | 03/17/2000 |
| SEQ ID NO:3750 | 60/190,479 | 03/17/2000 |
| SEQ ID NO:3751 | 60/190,479 | 03/17/2000 |
| SEQ ID NO:3752 | 60/190,479 | 03/17/2000 |
| SEQ ID NO:3753 | 60/190,479 | 03/17/2000 |
| SEQ ID NO:3754 | 60/190,479 | 03/17/2000 |
| SEQ ID NO:3755 | 60/190,479 | 03/17/2000 |
| SEQ ID NO:3756 | 60/190,479 | 03/17/2000 |
| SEQ ID NO:3757 | 60/190,479 | 03/17/2000 |
| SEQ ID NO:3758 | 60/190,479 | 03/17/2000 |
| SEQ ID NO:3759 | 60/190,479 | 03/17/2000 |
| SEQ ID NO:3760 | 60/190,479 | 03/17/2000 |
| SEQ ID NO:3761 | 60/190,479 | 03/17/2000 |
| SEQ ID NO:3762 | 60/190,479 | 03/17/2000 |
| SEQ ID NO:3763 | 60/190,479 | 03/17/2000 |
| SEQ ID NO:3764 | 60/190,479 | 03/17/2000 |
| SEQ ID NO:3765 | 60/190,479 | 03/17/2000 |
| SEQ ID NO:3766 | 60/190,479 | 03/17/2000 |
| SEQ ID NO:3767 | 60/190,479 | 03/17/2000 |
| SEQ ID NO:3768 | 60/190,479 | 03/17/2000 |
| SEQ ID NO:3769 | 60/190,479 | 03/17/2000 |
| SEQ ID NO:3770 | 60/190,479 | 03/17/2000 |
| SEQ ID NO:3771 | 60/190,479 | 03/17/2000 |
| SEQ ID NO:3772 | 60/190,479 | 03/17/2000 |
| SEQ ID NO:3773 | 60/190,479 | 03/17/2000 |
| SEQ ID NO:3774 | 60/190,479 | 03/17/2000 |
| SEQ ID NO:3775 | 60/190,479 | 03/17/2000 |
| SEQ ID NO:3776 | 60/190,479 | 03/17/2000 |
| SEQ ID NO:3777 | 60/190,479 | 03/17/2000 |
| SEQ ID NO:3778 | 60/190,479 | 03/17/2000 |
| SEQ ID NO:3779 | 60/190,479 | 03/17/2000 |
| SEQ ID NO:3780 | 60/190,479 | 03/17/2000 |
| SEQ ID NO:3781 | 60/190,479 | 03/17/2000 |
| SEQ ID NO:3782 | 60/190,479 | 03/17/2000 |
| SEQ ID NO:3783 | 60/190,479 | 03/17/2000 |
| SEQ ID NO:3784 | 60/190,479 | 03/17/2000 |
| SEQ ID NO:3785 | 60/190,479 | 03/17/2000 |
| SEQ ID NO:3786 | 60/190,479 | 03/17/2000 |
| SEQ ID NO:3787 | 60/190,479 | 03/17/2000 |
| SEQ ID NO:3788 | 60/190,479 | 03/17/2000 |
| SEQ ID NO:3789 | 60/190,479 | 03/17/2000 |
| SEQ ID NO:3790 | 60/190,479 | 03/17/2000 |
| SEQ ID NO:3791 | 60/190,479 | 03/17/2000 |
| SEQ ID NO:3792 | 60/190,479 | 03/17/2000 |
| SEQ ID NO:3793 | 60/190,479 | 03/17/2000 |
| SEQ ID NO:3794 | 60/190,479 | 03/17/2000 |
| SEQ ID NO:3795 | 60/190,479 | 03/17/2000 |
| SEQ ID NO:3796 | 60/190,479 | 03/17/2000 |
| SEQ ID NO:3797 | 60/190,479 | 03/17/2000 |
| SEQ ID NO:3798 | 60/190,479 | 03/17/2000 |
| SEQ ID NO:3799 | 60/190,479 | 03/17/2000 |
| SEQ ID NO:3800 | 60/190,479 | 03/17/2000 |
| SEQ ID NO:3801 | 60/190,479 | 03/17/2000 |
| SEQ ID NO:3802 | 60/190,479 | 03/17/2000 |
| SEQ ID NO:3803 | 60/190,479 | 03/17/2000 |
| SEQ ID NO:3804 | 60/190,479 | 03/17/2000 |
| SEQ ID NO:3805 | 60/190,479 | 03/17/2000 |
| SEQ ID NO:3806 | 60/190,479 | 03/17/2000 |
| SEQ ID NO:3807 | 60/190,479 | 03/17/2000 |
| SEQ ID NO:3808 | 60/190,479 | 03/17/2000 |
| SEQ ID NO:3809 | 60/190,479 | 03/17/2000 |
| SEQ ID NO:3810 | 60/190,479 | 03/17/2000 |
| SEQ ID NO:3811 | 60/190,479 | 03/17/2000 |
| SEQ ID NO:3812 | 60/190,479 | 03/17/2000 |
| SEQ ID NO:3813 | 60/190,479 | 03/17/2000 |
| SEQ ID NO:3814 | 60/190,479 | 03/17/2000 |
| SEQ ID NO:3815 | 60/190,479 | 03/17/2000 |
| SEQ ID NO:3816 | 60/190,479 | 03/17/2000 |
| SEQ ID NO:3817 | 60/190,479 | 03/17/2000 |
| SEQ ID NO:3818 | 60/190,479 | 03/17/2000 |
| SEQ ID NO:3819 | 60/190,479 | 03/17/2000 |
| SEQ ID NO:3820 | 60/190,479 | 03/17/2000 |
| SEQ ID NO:3821 | 60/190,479 | 03/17/2000 |
| SEQ ID NO:3822 | 60/190,479 | 03/17/2000 |
| SEQ ID NO:3823 | 60/190,479 | 03/17/2000 |
| SEQ ID NO:3824 | 60/190,479 | 03/17/2000 |
| SEQ ID NO:3825 | 60/190,479 | 03/17/2000 |
| SEQ ID NO:3826 | 60/190,479 | 03/17/2000 |
| SEQ ID NO:3827 | 60/190,479 | 03/17/2000 |
| SEQ ID NO:3828 | 60/190,479 | 03/17/2000 |
| SEQ ID NO:3829 | 60/190,479 | 03/17/2000 |
| SEQ ID NO:3830 | 60/190,479 | 03/17/2000 |
| SEQ ID NO:3831 | 60/190,479 | 03/17/2000 |
| SEQ ID NO:3832 | 60/190,479 | 03/17/2000 |
| SEQ ID NO:3833 | 60/190,479 | 03/17/2000 |
| SEQ ID NO:3834 | 60/190,479 | 03/17/2000 |
| SEQ ID NO:3835 | 60/190,479 | 03/17/2000 |
| SEQ ID NO:3836 | 60/190,479 | 03/17/2000 |
| SEQ ID NO:3837 | 60/190,479 | 03/17/2000 |
| SEQ ID NO:3838 | 60/190,479 | 03/17/2000 |
| SEQ ID NO:3839 | 60/190,479 | 03/17/2000 |
| SEQ ID NO:3840 | 60/190,479 | 03/17/2000 |
| SEQ ID NO:3841 | 60/190,479 | 03/17/2000 |
| SEQ ID NO:3842 | 60/190,479 | 03/17/2000 |
| SEQ ID NO:3843 | 60/190,479 | 03/17/2000 |
| SEQ ID NO:3844 | 60/190,479 | 03/17/2000 |
| SEQ ID NO:3845 | 60/190,479 | 03/17/2000 |
| SEQ ID NO:3846 | 60/190,479 | 03/17/2000 |
| SEQ ID NO:3847 | 60/190,479 | 03/17/2000 |
| SEQ ID NO:3848 | 60/190,479 | 03/17/2000 |
| SEQ ID NO:3849 | 60/190,479 | 03/17/2000 |
| SEQ ID NO:3850 | 60/190,479 | 03/17/2000 |
| SEQ ID NO:3851 | 60/190,479 | 03/17/2000 |
| SEQ ID NO:3852 | 60/190,479 | 03/17/2000 |
| SEQ ID NO:3853 | 60/190,479 | 03/17/2000 |
| SEQ ID NO:3854 | 60/190,479 | 03/17/2000 |
| SEQ ID NO:3855 | 60/190,479 | 03/17/2000 |
| SEQ ID NO:3856 | 60/190,479 | 03/17/2000 |
| SEQ ID NO:3857 | 60/190,479 | 03/17/2000 |
| SEQ ID NO:3858 | 60/190,479 | 03/17/2000 |
| SEQ ID NO:3859 | 60/190,479 | 03/17/2000 |
| SEQ ID NO:3860 | 60/190,479 | 03/17/2000 |
| SEQ ID NO:3861 | 60/190,479 | 03/17/2000 |
| SEQ ID NO:3862 | 60/190,479 | 03/17/2000 |
| SEQ ID NO:3863 | 60/190,479 | 03/17/2000 |
| SEQ ID NO:3864 | 60/190,479 | 03/17/2000 |
| SEQ ID NO:3865 | 60/190,479 | 03/17/2000 |
| SEQ ID NO:3866 | 60/190,479 | 03/17/2000 |
| SEQ ID NO:3867 | 60/190,479 | 03/17/2000 |
| SEQ ID NO:3868 | 60/190,479 | 03/17/2000 |
| SEQ ID NO:3869 | 60/190,479 | 03/17/2000 |
| SEQ ID NO:3870 | 60/190,479 | 03/17/2000 |
| SEQ ID NO:3871 | 60/190,479 | 03/17/2000 |
| SEQ ID NO:3872 | 60/190,479 | 03/17/2000 |
| SEQ ID NO:3873 | 60/190,479 | 03/17/2000 |
| SEQ ID NO:3874 | 60/190,479 | 03/17/2000 |
| SEQ ID NO:3875 | 60/190,479 | 03/17/2000 |
| SEQ ID NO:3876 | 60/190,479 | 03/17/2000 |
| SEQ ID NO:3877 | 60/190,479 | 03/17/2000 |
| SEQ ID NO:3878 | 60/190,479 | 03/17/2000 |
| SEQ ID NO:3879 | 60/190,479 | 03/17/2000 |
| SEQ ID NO:3880 | 60/190,479 | 03/17/2000 |
| SEQ ID NO:3881 | 60/190,479 | 03/17/2000 |
| SEQ ID NO:3882 | 60/190,479 | 03/17/2000 |
| SEQ ID NO:3883 | 60/190,479 | 03/17/2000 |
| SEQ ID NO:3884 | 60/190,479 | 03/17/2000 |
| SEQ ID NO:3885 | 60/190,479 | 03/17/2000 |
| SEQ ID NO:3886 | 60/190,479 | 03/17/2000 |
| SEQ ID NO:3887 | 60/190,479 | 03/17/2000 |
| SEQ ID NO:3888 | 60/223,416 | 08/04/2000 |
| SEQ ID NO:3889 | 60/223,416 | 08/04/2000 |
| SEQ ID NO:3890 | 60/223,416 | 08/04/2000 |
| SEQ ID NO:3891 | 60/223,416 | 08/04/2000 |
| SEQ ID NO:3892 | 60/223,416 | 08/04/2000 |
| SEQ ID NO:3893 | 60/223,416 | 08/04/2000 |
| SEQ ID NO:3894 | 60/223,416 | 08/04/2000 |
| SEQ ID NO:3895 | 60/223,416 | 08/04/2000 |
| SEQ ID NO:3896 | 60/223,416 | 08/04/2000 |
| SEQ ID NO:3897 | 60/223,416 | 08/04/2000 |
| SEQ ID NO:3898 | 60/223,416 | 08/04/2000 |
| SEQ ID NO:3899 | 60/223,416 | 08/04/2000 |
| SEQ ID NO:3900 | 60/223,416 | 08/04/2000 |
| SEQ ID NO:3901 | 60/223,416 | 08/04/2000 |
| SEQ ID NO:3902 | 60/223,416 | 08/04/2000 |
| SEQ ID NO:3903 | 60/223,416 | 08/04/2000 |
| SEQ ID NO:3904 | 60/223,416 | 08/04/2000 |
| SEQ ID NO:3905 | 60/223,416 | 08/04/2000 |
| SEQ ID NO:3906 | 60/223,416 | 08/04/2000 |
| SEQ ID NO:3907 | 60/223,416 | 08/04/2000 |
| SEQ ID NO:3908 | 60/223,416 | 08/04/2000 |
| SEQ ID NO:3909 | 60/223,416 | 08/04/2000 |
| SEQ ID NO:3910 | 60/223,416 | 08/04/2000 |
| SEQ ID NO:3911 | 60/223,416 | 08/04/2000 |
| SEQ ID NO:3912 | 60/223,416 | 08/04/2000 |
| SEQ ID NO:3913 | 60/223,416 | 08/04/2000 |
| SEQ ID NO:3914 | 60/223,416 | 08/04/2000 |
| SEQ ID NO:3915 | 60/223,416 | 08/04/2000 |
| SEQ ID NO:3916 | 60/223,416 | 08/04/2000 |
| SEQ ID NO:3917 | 60/223,416 | 08/04/2000 |
| SEQ ID NO:3918 | 60/223,416 | 08/04/2000 |
| SEQ ID NO:3919 | 60/223,416 | 08/04/2000 |
| SEQ ID NO:3920 | 60/223,416 | 08/04/2000 |
| SEQ ID NO:3921 | 60/223,416 | 08/04/2000 |
| SEQ ID NO:3922 | 60/223,416 | 08/04/2000 |
| SEQ ID NO:3923 | 60/223,416 | 08/04/2000 |
| SEQ ID NO:3924 | 60/223,416 | 08/04/2000 |
| SEQ ID NO:3925 | 60/223,416 | 08/04/2000 |
| SEQ ID NO:3926 | 60/223,416 | 08/04/2000 |
| SEQ ID NO:3927 | 60/223,416 | 08/04/2000 |
| SEQ ID NO:3928 | 60/223,416 | 08/04/2000 |
| SEQ ID NO:3929 | 60/223,416 | 08/04/2000 |
| SEQ ID NO:3930 | 60/223,416 | 08/04/2000 |
| SEQ ID NO:3931 | 60/223,416 | 08/04/2000 |
| SEQ ID NO:3932 | 60/223,416 | 08/04/2000 |
| SEQ ID NO:3933 | 60/223,416 | 08/04/2000 |
| SEQ ID NO:3934 | 60/223,416 | 08/04/2000 |
| SEQ ID NO:3935 | 60/223,416 | 08/04/2000 |
| SEQ ID NO:3936 | 60/223,416 | 08/04/2000 |
| SEQ ID NO:3937 | 60/223,416 | 08/04/2000 |
| SEQ ID NO:3938 | 60/223,416 | 08/04/2000 |
| SEQ ID NO:3939 | 60/223,416 | 08/04/2000 |
| SEQ ID NO:3940 | 60/223,416 | 08/04/2000 |
| SEQ ID NO:3941 | 60/223,416 | 08/04/2000 |
| SEQ ID NO:3942 | 60/223,416 | 08/04/2000 |
| SEQ ID NO:3943 | 60/223,416 | 08/04/2000 |
| SEQ ID NO:3944 | 60/223,416 | 08/04/2000 |
| SEQ ID NO:3945 | 60/223,416 | 08/04/2000 |
| SEQ ID NO:3946 | 60/223,416 | 08/04/2000 |
| SEQ ID NO:3947 | 60/223,416 | 08/04/2000 |
| SEQ ID NO:3948 | 60/223,416 | 08/04/2000 |
| SEQ ID NO:3949 | 60/223,416 | 08/04/2000 |
| SEQ ID NO:3950 | 60/223,416 | 08/04/2000 |
| SEQ ID NO:3951 | 60/223,416 | 08/04/2000 |
| SEQ ID NO:3952 | 60/223,416 | 08/04/2000 |
| SEQ ID NO:3953 | 60/223,416 | 08/04/2000 |
| SEQ ID NO:3954 | 60/223,416 | 08/04/2000 |
| SEQ ID NO:3955 | 60/223,416 | 08/04/2000 |
| SEQ ID NO:3956 | 60/223,416 | 08/04/2000 |
| SEQ ID NO:3957 | 60/223,416 | 08/04/2000 |
| SEQ ID NO:3958 | 60/223,416 | 08/04/2000 |
| SEQ ID NO:3959 | 60/223,416 | 08/04/2000 |
| SEQ ID NO:3960 | 60/223,416 | 08/04/2000 |
| SEQ ID NO:3961 | 60/223,416 | 08/04/2000 |
| SEQ ID NO:3962 | 60/223,416 | 08/04/2000 |
| SEQ ID NO:3963 | 60/223,416 | 08/04/2000 |
| SEQ ID NO:3964 | 60/223,416 | 08/04/2000 |
| SEQ ID NO:3965 | 60/223,416 | 08/04/2000 |
| SEQ ID NO:3966 | 60/223,416 | 08/04/2000 |
| SEQ ID NO:3967 | 60/223,416 | 08/04/2000 |
| SEQ ID NO:3968 | 60/223,416 | 08/04/2000 |
| SEQ ID NO:3969 | 60/223,416 | 08/04/2000 |
| SEQ ID NO:3970 | 60/223,416 | 08/04/2000 |
| SEQ ID NO:3971 | 60/223,416 | 08/04/2000 |
| SEQ ID NO:3972 | 60/223,416 | 08/04/2000 |
| SEQ ID NO:3973 | 60/223,416 | 08/04/2000 |
| SEQ ID NO:3974 | 60/223,416 | 08/04/2000 |
| SEQ ID NO:3975 | 60/223,416 | 08/04/2000 |
| SEQ ID NO:3976 | 60/223,416 | 08/04/2000 |
| SEQ ID NO:3977 | 60/223,416 | 08/04/2000 |
| SEQ ID NO:3978 | 60/223,416 | 08/04/2000 |
| SEQ ID NO:3979 | 60/223,416 | 08/04/2000 |
| SEQ ID NO:3980 | 60/223,416 | 08/04/2000 |
| SEQ ID NO:3981 | 60/223,416 | 08/04/2000 |
| SEQ ID NO:3982 | 60/223,416 | 08/04/2000 |
| SEQ ID NO:3983 | 60/223,416 | 08/04/2000 |
| SEQ ID NO:3984 | 60/223,416 | 08/04/2000 |
| SEQ ID NO:3985 | 60/223,416 | 08/04/2000 |
| SEQ ID NO:3986 | 60/223,416 | 08/04/2000 |
| SEQ ID NO:3987 | 60/223,416 | 08/04/2000 |
| SEQ ID NO:3988 | 60/223,416 | 08/04/2000 |
| SEQ ID NO:3989 | 60/223,416 | 08/04/2000 |
| SEQ ID NO:3990 | 60/223,416 | 08/04/2000 |
| SEQ ID NO:3991 | 60/223,416 | 08/04/2000 |
| SEQ ID NO:3992 | 60/223,416 | 08/04/2000 |
| SEQ ID NO:3993 | 60/223,416 | 08/04/2000 |
| SEQ ID NO:3994 | 60/223,416 | 08/04/2000 |
| SEQ ID NO:3995 | 60/223,416 | 08/04/2000 |
| SEQ ID NO:3996 | 60/223,416 | 08/04/2000 |
| SEQ ID NO:3997 | 60/223,416 | 08/04/2000 |
| SEQ ID NO:3998 | 60/223,416 | 08/04/2000 |
| SEQ ID NO:3999 | 60/223,416 | 08/04/2000 |
| SEQ ID NO:4000 | 60/223,416 | 08/04/2000 |
| SEQ ID NO:4001 | 60/223,416 | 08/04/2000 |
| SEQ ID NO:4002 | 60/223,416 | 08/04/2000 |
| SEQ ID NO:4003 | 60/223,416 | 08/04/2000 |
| SEQ ID NO:4004 | 60/223,416 | 08/04/2000 |
| SEQ ID NO:4005 | 60/223,416 | 08/04/2000 |
| SEQ ID NO:4006 | 60/223,416 | 08/04/2000 |
| SEQ ID NO:4007 | 60/223,416 | 08/04/2000 |
| SEQ ID NO:4008 | 60/223,416 | 08/04/2000 |
| SEQ ID NO:4009 | 60/223,416 | 08/04/2000 |
| SEQ ID NO:4010 | 60/223,416 | 08/04/2000 |
| SEQ ID NO:4011 | 60/223,416 | 08/04/2000 |
| SEQ ID NO:4012 | 60/223,416 | 08/04/2000 |
| SEQ ID NO:4013 | 60/223,416 | 08/04/2000 |
| SEQ ID NO:4014 | 60/223,416 | 08/04/2000 |
| SEQ ID NO:4015 | 60/223,416 | 08/04/2000 |
| SEQ ID NO:4016 | 60/223,416 | 08/04/2000 |
| SEQ ID NO:4017 | 60/223,416 | 08/04/2000 |
| SEQ ID NO:4018 | 60/223,416 | 08/04/2000 |
| SEQ ID NO:4019 | 60/223,416 | 08/04/2000 |
| SEQ ID NO:4020 | 60/223,416 | 08/04/2000 |
| SEQ ID NO:4021 | 60/223,416 | 08/04/2000 |
| SEQ ID NO:4022 | 60/223,416 | 08/04/2000 |
| SEQ ID NO:4023 | 60/223,416 | 08/04/2000 |
| SEQ ID NO:4024 | 60/223,416 | 08/04/2000 |
| SEQ ID NO:4025 | 60/223,416 | 08/04/2000 |
| SEQ ID NO:4026 | 60/223,416 | 08/04/2000 |
| SEQ ID NO:4027 | 60/223,416 | 08/04/2000 |
| SEQ ID NO:4028 | 60/223,416 | 08/04/2000 |
| SEQ ID NO:4029 | 60/223,416 | 08/04/2000 |
| SEQ ID NO:4030 | 60/223,416 | 08/04/2000 |
| SEQ ID NO:4031 | 60/223,416 | 08/04/2000 |
| SEQ ID NO:4032 | 60/223,416 | 08/04/2000 |
| SEQ ID NO:4033 | 60/223,416 | 08/04/2000 |
| SEQ ID NO:4034 | 60/223,416 | 08/04/2000 |
| SEQ ID NO:4035 | 60/223,416 | 08/04/2000 |
| SEQ ID NO:4036 | 60/223,416 | 08/04/2000 |
| SEQ ID NO:4037 | 60/223,416 | 08/04/2000 |
| SEQ ID NO:4038 | 60/223,416 | 08/04/2000 |
| SEQ ID NO:4039 | 60/223,416 | 08/04/2000 |
| SEQ ID NO:4040 | 60/223,416 | 08/04/2000 |
| SEQ ID NO:4041 | 60/223,416 | 08/04/2000 |
| SEQ ID NO:4042 | 60/223,416 | 08/04/2000 |
| SEQ ID NO:4043 | 60/223,416 | 08/04/2000 |
| SEQ ID NO:4044 | 60/223,416 | 08/04/2000 |
| SEQ ID NO:4045 | 60/223,416 | 08/04/2000 |
| SEQ ID NO:4046 | 60/223,416 | 08/04/2000 |
| SEQ ID NO:4047 | 60/223,416 | 08/04/2000 |
| SEQ ID NO:4048 | 60/223,416 | 08/04/2000 |
| SEQ ID NO:4049 | 60/223,416 | 08/04/2000 |
| SEQ ID NO:4050 | 60/223,416 | 08/04/2000 |
| SEQ ID NO:4051 | 60/223,416 | 08/04/2000 |
| SEQ ID NO:4052 | 60/223,416 | 08/04/2000 |
| SEQ ID NO:4053 | 60/223,416 | 08/04/2000 |
| SEQ ID NO:4054 | 60/223,416 | 08/04/2000 |
| SEQ ID NO:4055 | 60/223,416 | 08/04/2000 |
| SEQ ID NO:4056 | 60/223,416 | 08/04/2000 |
| SEQ ID NO:4057 | 60/223,416 | 08/04/2000 |
| SEQ ID NO:4058 | 60/223,416 | 08/04/2000 |
| SEQ ID NO:4059 | 60/223,416 | 08/04/2000 |
| SEQ ID NO:4060 | 60/223,416 | 08/04/2000 |
| SEQ ID NO:4061 | 60/223,416 | 08/04/2000 |
| SEQ ID NO:4062 | 60/223,416 | 08/04/2000 |
| SEQ ID NO:4063 | 60/223,416 | 08/04/2000 |
| SEQ ID NO:4064 | 60/223,416 | 08/04/2000 |
| SEQ ID NO:4065 | 60/223,416 | 08/04/2000 |
| SEQ ID NO:4066 | 60/223,416 | 08/04/2000 |
| SEQ ID NO:4067 | 60/223,416 | 08/04/2000 |
| SEQ ID NO:4068 | 60/223,416 | 08/04/2000 |
| SEQ ID NO:4069 | 60/223,416 | 08/04/2000 |
| SEQ ID NO:4070 | 60/223,416 | 08/04/2000 |
| SEQ ID NO:4071 | 60/223,416 | 08/04/2000 |
| SEQ ID NO:4072 | 60/223,416 | 08/04/2000 |
| SEQ ID NO:4073 | 60/223,416 | 08/04/2000 |
| SEQ ID NO:4074 | 60/223,416 | 08/04/2000 |
| SEQ ID NO:4075 | 60/223,416 | 08/04/2000 |
| SEQ ID NO:4076 | 60/223,416 | 08/04/2000 |
| SEQ ID NO:4077 | 60/223,416 | 08/04/2000 |
| SEQ ID NO:4078 | 60/223,416 | 08/04/2000 |
| SEQ ID NO:4079 | 60/223,416 | 08/04/2000 |
| SEQ ID NO:4080 | 60/223,416 | 08/04/2000 |
| SEQ ID NO:4081 | 60/223,416 | 08/04/2000 |
| SEQ ID NO:4082 | 60/223,416 | 08/04/2000 |
| SEQ ID NO:4083 | 60/223,416 | 08/04/2000 |
| SEQ ID NO:4084 | 60/223,416 | 08/04/2000 |
| SEQ ID NO:4085 | 60/223,416 | 08/04/2000 |
| SEQ ID NO:4086 | 60/223,416 | 08/04/2000 |
| SEQ ID NO:4087 | 60/223,416 | 08/04/2000 |
| SEQ ID NO:4088 | 60/223,416 | 08/04/2000 |
| SEQ ID NO:4089 | 60/223,416 | 08/04/2000 |
| SEQ ID NO:4090 | 60/223,416 | 08/04/2000 |
| SEQ ID NO:4091 | 60/223,416 | 08/04/2000 |
| SEQ ID NO:4092 | 60/223,416 | 08/04/2000 |
| SEQ ID NO:4093 | 60/223,416 | 08/04/2000 |
| SEQ ID NO:4094 | 60/223,416 | 08/04/2000 |
| SEQ ID NO:4095 | 60/223,416 | 08/04/2000 |
| SEQ ID NO:4096 | 60/223,416 | 08/04/2000 |
| SEQ ID NO:4097 | 60/223,416 | 08/04/2000 |
| SEQ ID NO:4098 | 60/223,416 | 08/04/2000 |
| SEQ ID NO:4099 | 60/223,416 | 08/04/2000 |
| SEQ ID NO:4100 | 60/223,416 | 08/04/2000 |
| SEQ ID NO:4101 | 60/223,416 | 08/04/2000 |
| SEQ ID NO:4102 | 60/223,416 | 08/04/2000 |
| SEQ ID NO:4103 | 60/223,416 | 08/04/2000 |
| SEQ ID NO:4104 | 60/223,416 | 08/04/2000 |
| SEQ ID NO:4105 | 60/223,416 | 08/04/2000 |
| SEQ ID NO:4106 | 60/223,416 | 08/04/2000 |
| SEQ ID NO:4107 | 60/223,416 | 08/04/2000 |
| SEQ ID NO:4108 | 60/223,416 | 08/04/2000 |
| SEQ ID NO:4109 | 60/223,416 | 08/04/2000 |
| SEQ ID NO:4110 | 60/223,416 | 08/04/2000 |
| SEQ ID NO:4111 | 60/223,416 | 08/04/2000 |
| SEQ ID NO:4112 | 60/223,416 | 08/04/2000 |
| SEQ ID NO:4113 | 60/223,416 | 08/04/2000 |
| SEQ ID NO:4114 | 60/223,416 | 08/04/2000 |
| SEQ ID NO:4115 | 60/223,416 | 08/04/2000 |
| SEQ ID NO:4116 | 60/223,416 | 08/04/2000 |
| SEQ ID NO:4117 | 60/223,416 | 08/04/2000 |
| SEQ ID NO:4118 | 60/223,416 | 08/04/2000 |
| SEQ ID NO:4119 | 60/223,416 | 08/04/2000 |
| SEQ ID NO:4120 | 60/223,416 | 08/04/2000 |
| SEQ ID NO:4121 | 60/223,416 | 08/04/2000 |
| SEQ ID NO:4122 | 60/223,416 | 08/04/2000 |
| SEQ ID NO:4123 | 60/223,416 | 08/04/2000 |
| SEQ ID NO:4124 | 60/223,416 | 08/04/2000 |
| SEQ ID NO:4125 | 60/223,416 | 08/04/2000 |
| SEQ ID NO:4126 | 60/223,416 | 08/04/2000 |
| SEQ ID NO:4127 | 60/223,416 | 08/04/2000 |
| SEQ ID NO:4128 | 60/223,416 | 08/04/2000 |
| SEQ ID NO:4129 | 60/223,416 | 08/04/2000 |
| SEQ ID NO:4130 | 60/223,416 | 08/04/2000 |
| SEQ ID NO:4131 | 60/223,416 | 08/04/2000 |
| SEQ ID NO:4132 | 60/223,416 | 08/04/2000 |
| SEQ ID NO:4133 | 60/223,416 | 08/04/2000 |
| SEQ ID NO:4134 | 60/223,416 | 08/04/2000 |
| SEQ ID NO:4135 | 60/223,416 | 08/04/2000 |
| SEQ ID NO:4136 | 60/223,416 | 08/04/2000 |
| SEQ ID NO:4137 | 60/223,416 | 08/04/2000 |
| SEQ ID NO:4138 | 60/223,416 | 08/04/2000 |
| SEQ ID NO:4139 | 60/223,416 | 08/04/2000 |
| SEQ ID NO:4140 | 60/223,416 | 08/04/2000 |
| SEQ ID NO:4141 | 60/223,416 | 08/04/2000 |
| SEQ ID NO:4142 | 60/223,416 | 08/04/2000 |
| SEQ ID NO:4143 | 60/223,416 | 08/04/2000 |
| SEQ ID NO:4144 | 60/223,416 | 08/04/2000 |
| SEQ ID NO:4145 | 60/223,416 | 08/04/2000 |
| SEQ ID NO:4146 | 60/223,416 | 08/04/2000 |
| SEQ ID NO:4147 | 60/223,416 | 08/04/2000 |
| SEQ ID NO:4148 | 60/223,416 | 08/04/2000 |
| SEQ ID NO:4149 | 60/223,416 | 08/04/2000 |
| SEQ ID NO:4150 | 60/223,416 | 08/04/2000 |
| SEQ ID NO:4151 | 60/223,416 | 08/04/2000 |
| SEQ ID NO:4152 | 60/223,416 | 08/04/2000 |
| SEQ ID NO:4153 | 60/223,416 | 08/04/2000 |
| SEQ ID NO:4154 | 60/223,416 | 08/04/2000 |
| SEQ ID NO:4155 | 60/223,416 | 08/04/2000 |
| SEQ ID NO:4156 | 60/223,416 | 08/04/2000 |
| SEQ ID NO:4157 | 60/223,416 | 08/04/2000 |
| SEQ ID NO:4158 | 60/223,416 | 08/04/2000 |
| SEQ ID NO:4159 | 60/223,416 | 08/04/2000 |
| SEQ ID NO:4160 | 60/223,416 | 08/04/2000 |
| SEQ ID NO:4161 | 60/223,416 | 08/04/2000 |
| SEQ ID NO:4162 | 60/223,416 | 08/04/2000 |
| SEQ ID NO:4163 | 60/223,416 | 08/04/2000 |
| SEQ ID NO:4164 | 60/223,416 | 08/04/2000 |
| SEQ ID NO:4165 | 60/223,416 | 08/04/2000 |
| SEQ ID NO:4166 | 60/223,416 | 08/04/2000 |
| SEQ ID NO:4167 | 60/223,416 | 08/04/2000 |
| SEQ ID NO:4168 | 60/223,416 | 08/04/2000 |
| SEQ ID NO:4169 | 60/223,416 | 08/04/2000 |
| SEQ ID NO:4170 | 60/223,416 | 08/04/2000 |
| SEQ ID NO:4171 | 60/223,416 | 08/04/2000 |
| SEQ ID NO:4172 | 60/223,416 | 08/04/2000 |
| SEQ ID NO:4173 | 60/223,416 | 08/04/2000 |
| SEQ ID NO:4174 | 60/223,416 | 08/04/2000 |
| SEQ ID NO:4175 | 60/223,416 | 08/04/2000 |
| SEQ ID NO:4176 | 60/223,416 | 08/04/2000 |
| SEQ ID NO:4177 | 60/223,416 | 08/04/2000 |
| SEQ ID NO:4178 | 60/223,416 | 08/04/2000 |
| SEQ ID NO:4179 | 60/223,416 | 08/04/2000 |
| SEQ ID NO:4180 | 60/223,416 | 08/04/2000 |
| SEQ ID NO:4181 | 60/223,416 | 08/04/2000 |
| SEQ ID NO:4182 | 60/223,416 | 08/04/2000 |
| SEQ ID NO:4183 | 60/223,416 | 08/04/2000 |
| SEQ ID NO:4184 | 60/223,416 | 08/04/2000 |
| SEQ ID NO:4185 | 60/223,416 | 08/04/2000 |
| SEQ ID NO:4186 | 60/223,416 | 08/04/2000 |
| SEQ ID NO:4187 | 60/223,416 | 08/04/2000 |
| SEQ ID NO:4188 | 60/223,416 | 08/04/2000 |
| SEQ ID NO:4189 | 60/223,416 | 08/04/2000 |
| SEQ ID NO:4190 | 60/223,416 | 08/04/2000 |
| SEQ ID NO:4191 | 60/223,416 | 08/04/2000 |
| SEQ ID NO:4192 | 60/223,416 | 08/04/2000 |
| SEQ ID NO:4193 | 60/223,416 | 08/04/2000 |
| SEQ ID NO:4194 | 60/223,416 | 08/04/2000 |
| SEQ ID NO:4195 | 60/223,416 | 08/04/2000 |
| SEQ ID NO:4196 | 60/223,416 | 08/04/2000 |
| SEQ ID NO:4197 | 60/223,416 | 08/04/2000 |
| SEQ ID NO:4198 | 60/223,416 | 08/04/2000 |
| SEQ ID NO:4199 | 60/223,416 | 08/04/2000 |
| SEQ ID NO:4200 | 60/223,416 | 08/04/2000 |
| SEQ ID NO:4201 | 60/223,416 | 08/04/2000 |
| SEQ ID NO:4202 | 60/223,416 | 08/04/2000 |
| SEQ ID NO:4203 | 60/223,416 | 08/04/2000 |
| SEQ ID NO:4204 | 60/223,416 | 08/04/2000 |
| SEQ ID NO:4205 | 60/223,416 | 08/04/2000 |
| SEQ ID NO:4206 | 60/223,416 | 08/04/2000 |
| SEQ ID NO:4207 | 60/223,416 | 08/04/2000 |
| SEQ ID NO:4208 | 60/223,416 | 08/04/2000 |
| SEQ ID NO:4209 | 60/223,416 | 08/04/2000 |
| SEQ ID NO:4210 | 60/223,416 | 08/04/2000 |
| SEQ ID NO:4211 | 60/223,416 | 08/04/2000 |
| SEQ ID NO:4212 | 60/223,416 | 08/04/2000 |
| SEQ ID NO:4213 | 60/223,416 | 08/04/2000 |
| SEQ ID NO:4214 | 60/223,416 | 08/04/2000 |
| SEQ ID NO:4215 | 60/223,416 | 08/04/2000 |
| SEQ ID NO:4216 | 60/223,416 | 08/04/2000 |
| SEQ ID NO:4217 | 60/223,416 | 08/04/2000 |
| SEQ ID NO:4218 | 60/223,416 | 08/04/2000 |
| SEQ ID NO:4219 | 60/223,416 | 08/04/2000 |
| SEQ ID NO:4220 | 60/223,416 | 08/04/2000 |
| SEQ ID NO:4221 | 60/223,416 | 08/04/2000 |
| SEQ ID NO:4222 | 60/223,416 | 08/04/2000 |
| SEQ ID NO:4223 | 60/223,416 | 08/04/2000 |
| SEQ ID NO:4224 | 60/223,416 | 08/04/2000 |
| SEQ ID NO:4225 | 60/223,416 | 08/04/2000 |
| SEQ ID NO:4226 | 60/223,416 | 08/04/2000 |
| SEQ ID NO:4227 | 60/223,416 | 08/04/2000 |
| SEQ ID NO:4228 | 60/223,416 | 08/04/2000 |
| SEQ ID NO:4229 | 60/223,416 | 08/04/2000 |
| SEQ ID NO:4230 | 60/223,416 | 08/04/2000 |
| SEQ ID NO:4231 | 60/223,416 | 08/04/2000 |
| SEQ ID NO:4232 | 60/223,416 | 08/04/2000 |
| SEQ ID NO:4233 | 60/223,416 | 08/04/2000 |
| SEQ ID NO:4234 | 60/223,416 | 08/04/2000 |
| SEQ ID NO:4235 | 60/223,416 | 08/04/2000 |
| SEQ ID NO:4236 | 60/223,416 | 08/04/2000 |
| SEQ ID NO:4237 | 60/223,416 | 08/04/2000 |
| SEQ ID NO:4238 | 60/223,416 | 08/04/2000 |
| SEQ ID NO:4239 | 60/223,416 | 08/04/2000 |
| SEQ ID NO:4240 | 60/223,416 | 08/04/2000 |
| SEQ ID NO:4241 | 60/223,416 | 08/04/2000 |
| SEQ ID NO:4242 | 60/223,416 | 08/04/2000 |
| SEQ ID NO:4243 | 60/223,416 | 08/04/2000 |
| SEQ ID NO:4244 | 60/223,416 | 08/04/2000 |
| SEQ ID NO:4245 | 60/223,416 | 08/04/2000 |
| SEQ ID NO:4246 | 60/223,416 | 08/04/2000 |
| SEQ ID NO:4247 | 60/223,416 | 08/04/2000 |
| SEQ ID NO:4248 | 60/223,416 | 08/04/2000 |
| SEQ ID NO:4249 | 60/223,416 | 08/04/2000 |
| SEQ ID NO:4250 | 60/223,416 | 08/04/2000 |
| SEQ ID NO:4251 | 60/223,416 | 08/04/2000 |
| SEQ ID NO:4252 | 60/223,416 | 08/04/2000 |
| SEQ ID NO:4253 | 60/223,416 | 08/04/2000 |
| SEQ ID NO:4254 | 60/223,416 | 08/04/2000 |
| SEQ ID NO:4255 | 60/223,416 | 08/04/2000 |
| SEQ ID NO:4256 | 60/223,416 | 08/04/2000 |
| SEQ ID NO:4257 | 60/223,416 | 08/04/2000 |
| SEQ ID NO:4258 | 60/223,416 | 08/04/2000 |
| SEQ ID NO:4259 | 60/223,416 | 08/04/2000 |
| SEQ ID NO:4260 | 60/223,416 | 08/04/2000 |
| SEQ ID NO:4261 | 60/223,416 | 08/04/2000 |
| SEQ ID NO:4262 | 60/223,416 | 08/04/2000 |
| SEQ ID NO:4263 | 60/223,416 | 08/04/2000 |
| SEQ ID NO:4264 | 60/223,416 | 08/04/2000 |
| SEQ ID NO:4265 | 60/223,416 | 08/04/2000 |
| SEQ ID NO:4266 | 60/223,416 | 08/04/2000 |
| SEQ ID NO:4267 | 60/223,416 | 08/04/2000 |
| SEQ ID NO:4268 | 60/223,416 | 08/04/2000 |
| SEQ ID NO:4269 | 60/223,416 | 08/04/2000 |
| SEQ ID NO:4270 | 60/223,416 | 08/04/2000 |
| SEQ ID NO:4271 | 60/223,416 | 08/04/2000 |
| SEQ ID NO:4272 | 60/223,416 | 08/04/2000 |
| SEQ ID NO:4273 | 60/223,416 | 08/04/2000 |
| SEQ ID NO:4274 | 60/223,416 | 08/04/2000 |
| SEQ ID NO:4275 | 60/223,416 | 08/04/2000 |
| SEQ ID NO:4276 | 60/223,416 | 08/04/2000 |
| SEQ ID NO:4277 | 60/223,416 | 08/04/2000 |
| SEQ ID NO:4278 | 60/223,416 | 08/04/2000 |
| SEQ ID NO:4279 | 60/223,416 | 08/04/2000 |
| SEQ ID NO:4280 | 60/223,416 | 08/04/2000 |
| SEQ ID NO:4281 | 60/223,416 | 08/04/2000 |
| SEQ ID NO:4282 | 60/223,416 | 08/04/2000 |
| SEQ ID NO:4283 | 60/223,416 | 08/04/2000 |
| SEQ ID NO:4284 | 60/223,416 | 08/04/2000 |
| SEQ ID NO:4285 | 60/223,416 | 08/04/2000 |
| SEQ ID NO:4286 | 60/223,416 | 08/04/2000 |
| SEQ ID NO:4287 | 60/223,416 | 08/04/2000 |
| SEQ ID NO:4288 | 60/223,416 | 08/04/2000 |
| SEQ ID NO:4289 | 60/223,416 | 08/04/2000 |
| SEQ ID NO:4290 | 60/223,416 | 08/04/2000 |
| SEQ ID NO:4291 | 60/223,416 | 08/04/2000 |
| SEQ ID NO:4292 | 60/223,416 | 08/04/2000 |
| SEQ ID NO:4293 | 60/223,416 | 08/04/2000 |
| SEQ ID NO:4294 | 60/223,416 | 08/04/2000 |
| SEQ ID NO:4295 | 60/223,416 | 08/04/2000 |
| SEQ ID NO:4296 | 60/223,416 | 08/04/2000 |
| SEQ ID NO:4297 | 60/223,416 | 08/04/2000 |
| SEQ ID NO:4298 | 60/223,416 | 08/04/2000 |
| SEQ ID NO:4299 | 60/223,416 | 08/04/2000 |
| SEQ ID NO:4300 | 60/223,416 | 08/04/2000 |
| SEQ ID NO:4301 | 60/223,416 | 08/04/2000 |
| SEQ ID NO:4302 | 60/223,416 | 08/04/2000 |
| SEQ ID NO:4303 | 60/223,416 | 08/04/2000 |
| SEQ ID NO:4304 | 60/223,416 | 08/04/2000 |
| SEQ ID NO:4305 | 60/223,416 | 08/04/2000 |
| SEQ ID NO:4306 | 60/223,416 | 08/04/2000 |
| SEQ ID NO:4307 | 60/223,416 | 08/04/2000 |
| SEQ ID NO:4308 | 60/223,416 | 08/04/2000 |
| SEQ ID NO:4309 | 60/223,416 | 08/04/2000 |
| SEQ ID NO:4310 | 60/223,416 | 08/04/2000 |
| SEQ ID NO:4311 | 60/223,416 | 08/04/2000 |
| SEQ ID NO:4312 | 60/223,416 | 08/04/2000 |
| SEQ ID NO:4313 | 60/223,416 | 08/04/2000 |
| SEQ ID NO:4314 | 60/223,416 | 08/04/2000 |
| SEQ ID NO:4315 | 60/223,416 | 08/04/2000 |
| SEQ ID NO:4316 | 60/223,416 | 08/04/2000 |
| SEQ ID NO:4317 | 60/223,416 | 08/04/2000 |
| SEQ ID NO:4318 | 60/223,416 | 08/04/2000 |
| SEQ ID NO:4319 | 60/223,416 | 08/04/2000 |
| SEQ ID NO:4320 | 60/223,416 | 08/04/2000 |
| SEQ ID NO:4321 | 60/223,416 | 08/04/2000 |
| SEQ ID NO:4322 | 60/223,416 | 08/04/2000 |
| SEQ ID NO:4323 | 60/223,416 | 08/04/2000 |
| SEQ ID NO:4324 | 60/223,416 | 08/04/2000 |
| SEQ ID NO:4325 | 60/223,416 | 08/04/2000 |
| SEQ ID NO:4326 | 60/223,416 | 08/04/2000 |
| SEQ ID NO:4327 | 60/223,416 | 08/04/2000 |
| SEQ ID NO:4328 | 60/223,416 | 08/04/2000 |
| SEQ ID NO:4329 | 60/223,416 | 08/04/2000 |
| SEQ ID NO:4330 | 60/223,416 | 08/04/2000 |
| SEQ ID NO:4331 | 60/223,416 | 08/04/2000 |
| SEQ ID NO:4332 | 60/223,416 | 08/04/2000 |
| SEQ ID NO:4333 | 60/223,416 | 08/04/2000 |
| SEQ ID NO:4334 | 60/223,416 | 08/04/2000 |
| SEQ ID NO:4335 | 60/223,416 | 08/04/2000 |
| SEQ ID NO:4336 | 60/223,416 | 08/04/2000 |
| SEQ ID NO:4337 | 60/223,416 | 08/04/2000 |
| SEQ ID NO:4338 | 60/223,416 | 08/04/2000 |
| SEQ ID NO:4339 | 60/223,416 | 08/04/2000 |
| SEQ ID NO:4340 | 60/223,416 | 08/04/2000 |
| SEQ ID NO:4341 | 60/223,416 | 08/04/2000 |
| SEQ ID NO:4342 | 60/223,416 | 08/04/2000 |
| SEQ ID NO:4343 | 60/223,416 | 08/04/2000 |
| SEQ ID NO:4344 | 60/223,416 | 08/04/2000 |
| SEQ ID NO:4345 | 60/223,416 | 08/04/2000 |
| SEQ ID NO:4346 | 60/223,416 | 08/04/2000 |
| SEQ ID NO:4347 | 60/223,416 | 08/04/2000 |
| SEQ ID NO:4348 | 60/223,416 | 08/04/2000 |
| SEQ ID NO:4349 | 60/223,416 | 08/04/2000 |
| SEQ ID NO:4350 | 60/223,416 | 08/04/2000 |
| SEQ ID NO:4351 | 60/223,416 | 08/04/2000 |
| SEQ ID NO:4352 | 60/223,416 | 08/04/2000 |
| SEQ ID NO:4353 | 60/223,416 | 08/04/2000 |
| SEQ ID NO:4354 | 60/223,416 | 08/04/2000 |
| SEQ ID NO:4355 | 60/223,416 | 08/04/2000 |
| SEQ ID NO:4356 | 60/223,416 | 08/04/2000 |
| SEQ ID NO:4357 | 60/223,416 | 08/04/2000 |
| SEQ ID NO:4358 | 60/223,416 | 08/04/2000 |
| SEQ ID NO:4359 | 60/223,416 | 08/04/2000 |
| SEQ ID NO:4360 | 60/223,416 | 08/04/2000 |
| SEQ ID NO:4361 | 60/223,416 | 08/04/2000 |
| SEQ ID NO:4362 | 60/223,416 | 08/04/2000 |
| SEQ ID NO:4363 | 60/223,416 | 08/04/2000 |
| SEQ ID NO:4364 | 60/223,416 | 08/04/2000 |
| SEQ ID NO:4365 | 60/223,416 | 08/04/2000 |
| SEQ ID NO:4366 | 60/223,416 | 08/04/2000 |
| SEQ ID NO:4367 | 60/223,416 | 08/04/2000 |
| SEQ ID NO:4368 | 60/223,416 | 08/04/2000 |
| SEQ ID NO:4369 | 60/223,416 | 08/04/2000 |
| SEQ ID NO:4370 | 60/223,416 | 08/04/2000 |
| SEQ ID NO:4371 | 60/223,416 | 08/04/2000 |
| SEQ ID NO:4372 | 60/223,416 | 08/04/2000 |
| SEQ ID NO:4373 | 60/223,416 | 08/04/2000 |
| SEQ ID NO:4374 | 60/223,416 | 08/04/2000 |
| SEQ ID NO:4375 | 60/223,416 | 08/04/2000 |
| SEQ ID NO:4376 | 60/223,416 | 08/04/2000 |
| SEQ ID NO:4377 | 60/223,416 | 08/04/2000 |
| SEQ ID NO:4378 | 60/223,416 | 08/04/2000 |
| SEQ ID NO:4379 | 60/223,416 | 08/04/2000 |
| SEQ ID NO:4380 | 60/223,416 | 08/04/2000 |
| SEQ ID NO:4381 | 60/223,416 | 08/04/2000 |
| SEQ ID NO:4382 | 60/223,416 | 08/04/2000 |
| SEQ ID NO:4383 | 60/223,416 | 08/04/2000 |
| SEQ ID NO:4384 | 60/223,416 | 08/04/2000 |
| SEQ ID NO:4385 | 60/223,416 | 08/04/2000 |
| SEQ ID NO:4386 | 60/223,416 | 08/04/2000 |
| SEQ ID NO:4387 | 60/223,416 | 08/04/2000 |
| SEQ ID NO:4388 | 60/223,416 | 08/04/2000 |
| SEQ ID NO:4389 | 60/223,416 | 08/04/2000 |
| SEQ ID NO:4390 | 60/223,416 | 08/04/2000 |
| SEQ ID NO:4391 | 60/223,416 | 08/04/2000 |
| SEQ ID NO:4392 | 60/223,416 | 08/04/2000 |
| SEQ ID NO:4393 | 60/223,416 | 08/04/2000 |
| SEQ ID NO:4394 | 60/223,416 | 08/04/2000 |
| SEQ ID NO:4395 | 60/223,416 | 08/04/2000 |
| SEQ ID NO:4396 | 60/223,416 | 08/04/2000 |
| SEQ ID NO:4397 | 60/223,416 | 08/04/2000 |
| SEQ ID NO:4398 | 60/223,416 | 08/04/2000 |
| SEQ ID NO:4399 | 60/223,416 | 08/04/2000 |
| SEQ ID NO:4400 | 60/223,416 | 08/04/2000 |
| SEQ ID NO:4401 | 60/223,416 | 08/04/2000 |
| SEQ ID NO:4402 | 60/223,416 | 08/04/2000 |
| SEQ ID NO:4403 | 60/223,416 | 08/04/2000 |
| SEQ ID NO:4404 | 60/223,416 | 08/04/2000 |
| SEQ ID NO:4405 | 60/223,416 | 08/04/2000 |
| SEQ ID NO:4406 | 60/223,416 | 08/04/2000 |
| SEQ ID NO:4407 | 60/223,416 | 08/04/2000 |
| SEQ ID NO:4408 | 60/223,416 | 08/04/2000 |
| SEQ ID NO:4409 | 60/223,416 | 08/04/2000 |
| SEQ ID NO:4410 | 60/223,416 | 08/04/2000 |
| SEQ ID NO:4411 | 60/223,416 | 08/04/2000 |
| SEQ ID NO:4412 | 60/223,416 | 08/04/2000 |
| SEQ ID NO:4413 | 60/223,416 | 08/04/2000 |
| SEQ ID NO:4414 | 60/223,416 | 08/04/2000 |
| SEQ ID NO:4415 | 60/223,416 | 08/04/2000 |
| SEQ ID NO:4416 | 60/223,416 | 08/04/2000 |
| SEQ ID NO:4417 | 60/223,416 | 08/04/2000 |
| SEQ ID NO:4418 | 60/223,416 | 08/04/2000 |
| SEQ ID NO:4419 | 60/223,416 | 08/04/2000 |
| SEQ ID NO:4420 | 60/223,416 | 08/04/2000 |
| SEQ ID NO:4421 | 60/223,416 | 08/04/2000 |
| SEQ ID NO:4422 | 60/223,416 | 08/04/2000 |
| SEQ ID NO:4423 | 60/223,416 | 08/04/2000 |
| SEQ ID NO:4424 | 60/223,416 | 08/04/2000 |
| SEQ ID NO:4425 | 60/223,416 | 08/04/2000 |
| SEQ ID NO:4426 | 60/223,416 | 08/04/2000 |
| SEQ ID NO:4427 | 60/223,416 | 08/04/2000 |
| SEQ ID NO:4428 | 60/223,416 | 08/04/2000 |
| SEQ ID NO:4429 | 60/223,416 | 08/04/2000 |
| SEQ ID NO:4430 | 60/223,416 | 08/04/2000 |
| SEQ ID NO:4431 | 60/223,416 | 08/04/2000 |
| SEQ ID NO:4432 | 60/223,416 | 08/04/2000 |
| SEQ ID NO:4433 | 60/223,416 | 08/04/2000 |
| SEQ ID NO:4434 | 60/223,416 | 08/04/2000 |
| SEQ ID NO:4435 | 60/223,416 | 08/04/2000 |
| SEQ ID NO:4436 | 60/223,416 | 08/04/2000 |
| SEQ ID NO:4437 | 60/223,416 | 08/04/2000 |
| SEQ ID NO:4438 | 60/223,416 | 08/04/2000 |
| SEQ ID NO:4439 | 60/223,416 | 08/04/2000 |
| SEQ ID NO:4440 | 60/223,416 | 08/04/2000 |
| SEQ ID NO:4441 | 60/223,416 | 08/04/2000 |
| SEQ ID NO:4442 | 60/223,416 | 08/04/2000 |
| SEQ ID NO:4443 | 60/223,416 | 08/04/2000 |
| SEQ ID NO:4444 | 60/223,416 | 08/04/2000 |
| SEQ ID NO:4445 | 60/223,416 | 08/04/2000 |
| SEQ ID NO:4446 | 60/223,416 | 08/04/2000 |
| SEQ ID NO:4447 | 60/223,416 | 08/04/2000 |
| SEQ ID NO:4448 | 60/223,416 | 08/04/2000 |
| SEQ ID NO:4449 | 60/223,416 | 08/04/2000 |
| SEQ ID NO:4450 | 60/223,416 | 08/04/2000 |
| SEQ ID NO:4451 | 60/223,416 | 08/04/2000 |
| SEQ ID NO:4452 | 60/223,416 | 08/04/2000 |
| SEQ ID NO:4453 | 60/223,416 | 08/04/2000 |
| SEQ ID NO:4454 | 60/223,416 | 08/04/2000 |
| SEQ ID NO:4455 | 60/223,416 | 08/04/2000 |
| SEQ ID NO:4456 | 60/223,416 | 08/04/2000 |
| SEQ ID NO:4457 | 60/223,416 | 08/04/2000 |
| SEQ ID NO:4458 | 60/223,416 | 08/04/2000 |
| SEQ ID NO:4459 | 60/223,416 | 08/04/2000 |
| SEQ ID NO:4460 | 60/223,416 | 08/04/2000 |
| SEQ ID NO:4461 | 60/223,416 | 08/04/2000 |
| SEQ ID NO:4462 | 60/223,416 | 08/04/2000 |
| SEQ ID NO:4463 | 60/223,416 | 08/04/2000 |
| SEQ ID NO:4464 | 60/223,416 | 08/04/2000 |
| SEQ ID NO:4465 | 60/223,416 | 08/04/2000 |
| SEQ ID NO:4466 | 60/223,416 | 08/04/2000 |
| SEQ ID NO:4467 | 60/223,416 | 08/04/2000 |
| SEQ ID NO:4468 | 60/223,416 | 08/04/2000 |
| SEQ ID NO:4469 | 60/223,416 | 08/04/2000 |
| SEQ ID NO:4470 | 60/223,416 | 08/04/2000 |
| SEQ ID NO:4471 | 60/223,416 | 08/04/2000 |
| SEQ ID NO:4472 | 60/223,416 | 08/04/2000 |
| SEQ ID NO:4473 | 60/223,416 | 08/04/2000 |
| SEQ ID NO:4474 | 60/223,416 | 08/04/2000 |
| SEQ ID NO:4475 | 60/223,416 | 08/04/2000 |
| SEQ ID NO:4476 | 60/223,416 | 08/04/2000 |
| SEQ ID NO:4477 | 60/223,416 | 08/04/2000 |
| SEQ ID NO:4478 | 60/223,416 | 08/04/2000 |
| SEQ ID NO:4479 | 60/223,416 | 08/04/2000 |
| SEQ ID NO:4480 | 60/223,416 | 08/04/2000 |
| SEQ ID NO:4481 | 60/223,416 | 08/04/2000 |
| SEQ ID NO:4482 | 60/223,416 | 08/04/2000 |
| SEQ ID NO:4483 | 60/223,416 | 08/04/2000 |
| SEQ ID NO:4484 | 60/223,416 | 08/04/2000 |
| SEQ ID NO:4485 | 60/223,416 | 08/04/2000 |
| SEQ ID NO:4486 | 60/223,416 | 08/04/2000 |
| SEQ ID NO:4487 | 60/223,416 | 08/04/2000 |
| SEQ ID NO:4488 | 60/223,416 | 08/04/2000 |
| SEQ ID NO:4489 | 60/223,416 | 08/04/2000 |
| SEQ ID NO:4490 | 60/223,416 | 08/04/2000 |
| SEQ ID NO:4491 | 60/223,416 | 08/04/2000 |
| SEQ ID NO:4492 | 60/223,416 | 08/04/2000 |
| SEQ ID NO:4493 | 60/223,416 | 08/04/2000 |
| SEQ ID NO:4494 | 60/223,416 | 08/04/2000 |
| SEQ ID NO:4495 | 60/223,416 | 08/04/2000 |
| SEQ ID NO:4496 | 60/223,416 | 08/04/2000 |
| SEQ ID NO:4497 | 60/223,416 | 08/04/2000 |
| SEQ ID NO:4498 | 60/223,416 | 08/04/2000 |
| SEQ ID NO:4499 | 60/223,416 | 08/04/2000 |
| SEQ ID NO:4500 | 60/223,416 | 08/04/2000 |
| SEQ ID NO:4501 | 60/223,416 | 08/04/2000 |
| SEQ ID NO:4502 | 60/223,416 | 08/04/2000 |
| SEQ ID NO:4503 | 60/223,416 | 08/04/2000 |
| SEQ ID NO:4504 | 60/223,416 | 08/04/2000 |
| SEQ ID NO:4505 | 60/223,416 | 08/04/2000 |
| SEQ ID NO:4506 | 60/223,416 | 08/04/2000 |
| SEQ ID NO:4507 | 60/223,416 | 08/04/2000 |
| SEQ ID NO:4508 | 60/223,416 | 08/04/2000 |
| SEQ ID NO:4509 | 60/223,416 | 08/04/2000 |
| SEQ ID NO:4510 | 60/223,416 | 08/04/2000 |
| SEQ ID NO:4511 | 60/223,416 | 08/04/2000 |
| SEQ ID NO:4512 | 60/223,416 | 08/04/2000 |
| SEQ ID NO:4513 | 60/223,416 | 08/04/2000 |
| SEQ ID NO:4514 | 60/223,416 | 08/04/2000 |
| SEQ ID NO:4515 | 60/223,416 | 08/04/2000 |
| SEQ ID NO:4516 | 60/223,416 | 08/04/2000 |
| SEQ ID NO:4517 | 60/223,416 | 08/04/2000 |
| SEQ ID NO:4518 | 60/223,416 | 08/04/2000 |
| SEQ ID NO:4519 | 60/223,416 | 08/04/2000 |
| SEQ ID NO:4520 | 60/223,416 | 08/04/2000 |
| SEQ ID NO:4521 | 60/223,416 | 08/04/2000 |
| SEQ ID NO:4522 | 60/223,416 | 08/04/2000 |
| SEQ ID NO:4523 | 60/223,416 | 08/04/2000 |
| SEQ ID NO:4524 | 60/223,416 | 08/04/2000 |
| SEQ ID NO:4525 | 60/223,416 | 08/04/2000 |
| SEQ ID NO:4526 | 60/223,416 | 08/04/2000 |
| SEQ ID NO:4527 | 60/223,416 | 08/04/2000 |
| SEQ ID NO:4528 | 60/223,416 | 08/04/2000 |
| SEQ ID NO:4529 | 60/223,416 | 08/04/2000 |
| SEQ ID NO:4530 | 60/223,416 | 08/04/2000 |
| SEQ ID NO:4531 | 60/223,416 | 08/04/2000 |
| SEQ ID NO:4532 | 60/223,416 | 08/04/2000 |
| SEQ ID NO:4533 | 60/223,416 | 08/04/2000 |
| SEQ ID NO:4534 | 60/223,416 | 08/04/2000 |
| SEQ ID NO:4535 | 60/223,416 | 08/04/2000 |
| SEQ ID NO:4536 | 60/223,416 | 08/04/2000 |
| SEQ ID NO:4537 | 60/223,416 | 08/04/2000 |
| SEQ ID NO:4538 | 60/223,416 | 08/04/2000 |
| SEQ ID NO:4539 | 60/223,416 | 08/04/2000 |
| SEQ ID NO:4540 | 60/223,416 | 08/04/2000 |
| SEQ ID NO:4541 | 60/223,416 | 08/04/2000 |
| SEQ ID NO:4542 | 60/223,416 | 08/04/2000 |
| SEQ ID NO:4543 | 60/223,416 | 08/04/2000 |
| SEQ ID NO:4544 | 60/223,416 | 08/04/2000 |
| SEQ ID NO:4545 | 60/223,416 | 08/04/2000 |
| SEQ ID NO:4546 | 60/223,416 | 08/04/2000 |
| SEQ ID NO:4547 | 60/223,416 | 08/04/2000 |
| SEQ ID NO:4548 | 60/223,416 | 08/04/2000 |
| SEQ ID NO:4549 | 60/223,416 | 08/04/2000 |
| SEQ ID NO:4550 | 60/223,416 | 08/04/2000 |
| SEQ ID NO:4551 | 60/223,416 | 08/04/2000 |
| SEQ ID NO:4552 | 60/223,416 | 08/04/2000 |
| SEQ ID NO:4553 | 60/223,416 | 08/04/2000 |
| SEQ ID NO:4554 | 60/223,416 | 08/04/2000 |
| SEQ ID NO:4555 | 60/223,416 | 08/04/2000 |
| SEQ ID NO:4556 | 60/223,416 | 08/04/2000 |
| SEQ ID NO:4557 | 60/223,416 | 08/04/2000 |
| SEQ ID NO:4558 | 60/223,416 | 08/04/2000 |
| SEQ ID NO:4559 | 60/223,416 | 08/04/2000 |
| SEQ ID NO:4560 | 60/223,416 | 08/04/2000 |
| SEQ ID NO:4561 | 60/223,416 | 08/04/2000 |
| SEQ ID NO:4562 | 60/223,416 | 08/04/2000 |
| SEQ ID NO:4563 | 60/223,416 | 08/04/2000 |
| SEQ ID NO:4564 | 60/223,416 | 08/04/2000 |
| SEQ ID NO:4565 | 60/223,416 | 08/04/2000 |
| SEQ ID NO:4566 | 60/223,416 | 08/04/2000 |
| SEQ ID NO:4567 | 60/223,416 | 08/04/2000 |
| SEQ ID NO:4568 | 60/223,416 | 08/04/2000 |
| SEQ ID NO:4569 | 60/223,416 | 08/04/2000 |
| SEQ ID NO:4570 | 60/223,416 | 08/04/2000 |
| SEQ ID NO:4571 | 60/223,416 | 08/04/2000 |
| SEQ ID NO:4572 | 60/223,416 | 08/04/2000 |
| SEQ ID NO:4573 | 60/223,416 | 08/04/2000 |
| SEQ ID NO:4574 | 60/223,416 | 08/04/2000 |
| SEQ ID NO:4575 | 60/223,416 | 08/04/2000 |
| SEQ ID NO:4576 | 60/223,416 | 08/04/2000 |
| SEQ ID NO:4577 | 60/223,416 | 08/04/2000 |
| SEQ ID NO:4578 | 60/223,416 | 08/04/2000 |
| SEQ ID NO:4579 | 60/223,416 | 08/04/2000 |
| SEQ ID NO:4580 | 60/223,416 | 08/04/2000 |
| SEQ ID NO:4581 | 60/223,416 | 08/04/2000 |
| SEQ ID NO:4582 | 60/223,416 | 08/04/2000 |
| SEQ ID NO:4583 | 60/223,416 | 08/04/2000 |
| SEQ ID NO:4584 | 60/223,416 | 08/04/2000 |
| SEQ ID NO:4585 | 60/223,416 | 08/04/2000 |
| SEQ ID NO:4586 | 60/223,416 | 08/04/2000 |
| SEQ ID NO:4587 | 60/223,416 | 08/04/2000 |
| SEQ ID NO:4588 | 60/223,416 | 08/04/2000 |
| SEQ ID NO:4589 | 60/223,416 | 08/04/2000 |
| SEQ ID NO:4590 | 60/223,416 | 08/04/2000 |
| SEQ ID NO:4591 | 60/223,416 | 08/04/2000 |
| SEQ ID NO:4592 | 60/223,416 | 08/04/2000 |
| SEQ ID NO:4593 | 60/223,416 | 08/04/2000 |
| SEQ ID NO:4594 | 60/223,416 | 08/04/2000 |
| SEQ ID NO:4595 | 60/223,416 | 08/04/2000 |
| SEQ ID NO:4596 | 60/223,416 | 08/04/2000 |
| SEQ ID NO:4597 | 60/223,416 | 08/04/2000 |
| SEQ ID NO:4598 | 60/223,416 | 08/04/2000 |
| SEQ ID NO:4599 | 60/223,416 | 08/04/2000 |
| SEQ ID NO:4600 | 60/223,416 | 08/04/2000 |
| SEQ ID NO:4601 | 60/223,416 | 08/04/2000 |
| SEQ ID NO:4602 | 60/223,416 | 08/04/2000 |
| SEQ ID NO:4603 | 60/223,416 | 08/04/2000 |
| SEQ ID NO:4604 | 60/223,416 | 08/04/2000 |
| SEQ ID NO:4605 | 60/223,416 | 08/04/2000 |
| SEQ ID NO:4606 | 60/223,416 | 08/04/2000 |
| SEQ ID NO:4607 | 60/223,416 | 08/04/2000 |
| SEQ ID NO:4608 | 60/223,416 | 08/04/2000 |
| SEQ ID NO:4609 | 60/223,416 | 08/04/2000 |
| SEQ ID NO:4610 | 60/223,416 | 08/04/2000 |
| SEQ ID NO:4611 | 60/223,416 | 08/04/2000 |
| SEQ ID NO:4612 | 60/223,416 | 08/04/2000 |
| SEQ ID NO:4613 | 60/223,416 | 08/04/2000 |
| SEQ ID NO:4614 | 60/223,416 | 08/04/2000 |
| SEQ ID NO:4615 | 60/223,416 | 08/04/2000 |
| SEQ ID NO:4616 | 60/223,416 | 08/04/2000 |
| SEQ ID NO:4617 | 60/223,416 | 08/04/2000 |
| SEQ ID NO:4618 | 60/223,416 | 08/04/2000 |
| SEQ ID NO:4619 | 60/223,416 | 08/04/2000 |
| SEQ ID NO:4620 | 60/223,416 | 08/04/2000 |
| SEQ ID NO:4621 | 60/223,416 | 08/04/2000 |
| SEQ ID NO:4622 | 60/223,416 | 08/04/2000 |
| SEQ ID NO:4623 | 60/223,416 | 08/04/2000 |
| SEQ ID NO:4624 | 60/223,416 | 08/04/2000 |
| SEQ ID NO:4625 | 60/223,416 | 08/04/2000 |
| SEQ ID NO:4626 | 60/223,416 | 08/04/2000 |
| SEQ ID NO:4627 | 60/223,416 | 08/04/2000 |
| SEQ ID NO:4628 | 60/223,416 | 08/04/2000 |
| SEQ ID NO:4629 | 60/223,416 | 08/04/2000 |
| SEQ ID NO:4630 | 60/223,416 | 08/04/2000 |
| SEQ ID NO:4631 | 60/223,416 | 08/04/2000 |
| SEQ ID NO:4632 | 60/223,416 | 08/04/2000 |
| SEQ ID NO:4633 | 60/223,416 | 08/04/2000 |
| SEQ ID NO:4634 | 60/223,416 | 08/04/2000 |
| SEQ ID NO:4635 | 60/223,416 | 08/04/2000 |
| SEQ ID NO:4636 | 60/223,416 | 08/04/2000 |
| SEQ ID NO:4637 | 60/223,416 | 08/04/2000 |
| SEQ ID NO:4638 | 60/223,416 | 08/04/2000 |
| SEQ ID NO:4639 | 60/223,416 | 08/04/2000 |
| SEQ ID NO:4640 | 60/223,416 | 08/04/2000 |
| SEQ ID NO:4641 | 60/223,416 | 08/04/2000 |
| SEQ ID NO:4642 | 60/223,416 | 08/04/2000 |
| SEQ ID NO:4643 | 60/223,416 | 08/04/2000 |
| SEQ ID NO:4644 | 60/223,416 | 08/04/2000 |
| SEQ ID NO:4645 | 60/223,416 | 08/04/2000 |
| SEQ ID NO:4646 | 60/223,416 | 08/04/2000 |
| SEQ ID NO:4647 | 60/223,416 | 08/04/2000 |
| SEQ ID NO:4648 | 60/223,416 | 08/04/2000 |
| SEQ ID NO:4649 | 60/223,416 | 08/04/2000 |
| SEQ ID NO:4650 | 60/223,416 | 08/04/2000 |
| SEQ ID NO:4651 | 60/223,416 | 08/04/2000 |
| SEQ ID NO:4652 | 60/223,416 | 08/04/2000 |
| SEQ ID NO:4653 | 60/223,416 | 08/04/2000 |
| SEQ ID NO:4654 | 60/223,416 | 08/04/2000 |
| SEQ ID NO:4655 | 60/223,416 | 08/04/2000 |
| SEQ ID NO:4656 | 60/223,416 | 08/04/2000 |
| SEQ ID NO:4657 | 60/223,416 | 08/04/2000 |
| SEQ ID NO:4658 | 60/223,416 | 08/04/2000 |
| SEQ ID NO:4659 | 60/223,416 | 08/04/2000 |
| SEQ ID NO:4660 | 60/223,416 | 08/04/2000 |
| SEQ ID NO:4661 | 60/223,416 | 08/04/2000 |
| SEQ ID NO:4662 | 60/223,416 | 08/04/2000 |
| SEQ ID NO:4663 | 60/223,416 | 08/04/2000 |
| SEQ ID NO:4664 | 60/223,416 | 08/04/2000 |
| SEQ ID NO:4665 | 60/223,416 | 08/04/2000 |
| SEQ ID NO:4666 | 60/223,416 | 08/04/2000 |
| SEQ ID NO:4667 | 60/223,416 | 08/04/2000 |
| SEQ ID NO:4668 | 60/223,416 | 08/04/2000 |
| SEQ ID NO:4669 | 60/223,416 | 08/04/2000 |
| SEQ ID NO:4670 | 60/223,416 | 08/04/2000 |
| SEQ ID NO:4671 | 60/223,416 | 08/04/2000 |
| SEQ ID NO:4672 | 60/223,416 | 08/04/2000 |
| SEQ ID NO:4673 | 60/223,416 | 08/04/2000 |
| SEQ ID NO:4674 | 60/223,416 | 08/04/2000 |
| SEQ ID NO:4675 | 60/223,416 | 08/04/2000 |
| SEQ ID NO:4676 | 60/223,416 | 08/04/2000 |
| SEQ ID NO:4677 | 60/223,416 | 08/04/2000 |
| SEQ ID NO:4678 | 60/223,416 | 08/04/2000 |
| SEQ ID NO:4679 | 60/223,416 | 08/04/2000 |
| SEQ ID NO:4680 | 60/223,416 | 08/04/2000 |
| SEQ ID NO:4681 | 60/223,416 | 08/04/2000 |
| SEQ ID NO:4682 | 60/223,416 | 08/04/2000 |
| SEQ ID NO:4683 | 60/223,416 | 08/04/2000 |
| SEQ ID NO:4684 | 60/223,416 | 08/04/2000 |
| SEQ ID NO:4685 | 60/223,416 | 08/04/2000 |
| SEQ ID NO:4686 | 60/223,416 | 08/04/2000 |
| SEQ ID NO:4687 | 60/223,416 | 08/04/2000 |
| SEQ ID NO:4688 | 60/223,416 | 08/04/2000 |
| SEQ ID NO:4689 | 60/223,416 | 08/04/2000 |
| SEQ ID NO:4690 | 60/223,416 | 08/04/2000 |
| SEQ ID NO:4691 | 60/223,416 | 08/04/2000 |
| SEQ ID NO:4692 | 60/223,416 | 08/04/2000 |
| SEQ ID NO:4693 | 60/223,416 | 08/04/2000 |
| SEQ ID NO:4694 | 60/223,416 | 08/04/2000 |
| SEQ ID NO:4695 | 60/223,416 | 08/04/2000 |
| SEQ ID NO:4696 | 60/223,416 | 08/04/2000 |
| SEQ ID NO:4697 | 60/223,416 | 08/04/2000 |
| SEQ ID NO:4698 | 60/223,416 | 08/04/2000 |
| SEQ ID NO:4699 | 60/223,416 | 08/04/2000 |
| SEQ ID NO:4700 | 60/223,416 | 08/04/2000 |
| SEQ ID NO:4701 | 60/223,416 | 08/04/2000 |
| SEQ ID NO:4702 | 60/223,416 | 08/04/2000 |
| SEQ ID NO:4703 | 60/223,416 | 08/04/2000 |
| SEQ ID NO:4704 | 60/223,416 | 08/04/2000 |
| SEQ ID NO:4705 | 60/223,416 | 08/04/2000 |
| SEQ ID NO:4706 | 60/223,416 | 08/04/2000 |
| SEQ ID NO:4707 | 60/223,416 | 08/04/2000 |
| SEQ ID NO:4708 | 60/223,416 | 08/04/2000 |
| SEQ ID NO:4709 | 60/223,416 | 08/04/2000 |
| SEQ ID NO:4710 | 60/223,416 | 08/04/2000 |
| SEQ ID NO:4711 | 60/223,416 | 08/04/2000 |
| SEQ ID NO:4712 | 60/223,416 | 08/04/2000 |
| SEQ ID NO:4713 | 60/223,416 | 08/04/2000 |
| SEQ ID NO:4714 | 60/223,416 | 08/04/2000 |
| SEQ ID NO:4715 | 60/223,416 | 08/04/2000 |
| SEQ ID NO:4716 | 60/223,416 | 08/04/2000 |
| SEQ ID NO:4717 | 60/223,416 | 08/04/2000 |
| SEQ ID NO:4718 | 60/223,416 | 08/04/2000 |
| SEQ ID NO:4719 | 60/223,416 | 08/04/2000 |
| SEQ ID NO:4720 | 60/223,416 | 08/04/2000 |
| SEQ ID NO:4721 | 60/223,416 | 08/04/2000 |
| SEQ ID NO:4722 | 60/223,416 | 08/04/2000 |
| SEQ ID NO:4723 | 60/223,416 | 08/04/2000 |
| SEQ ID NO:4724 | 60/223,416 | 08/04/2000 |
| SEQ ID NO:4725 | 60/223,416 | 08/04/2000 |
| SEQ ID NO:4726 | 60/223,416 | 08/04/2000 |
| SEQ ID NO:4727 | 60/223,416 | 08/04/2000 |
| SEQ ID NO:4728 | 60/223,416 | 08/04/2000 |
| SEQ ID NO:4729 | 60/223,416 | 08/04/2000 |
| SEQ ID NO:4730 | 60/223,416 | 08/04/2000 |
| SEQ ID NO:4731 | 60/223,416 | 08/04/2000 |
| SEQ ID NO:4732 | 60/223,416 | 08/04/2000 |
| SEQ ID NO:4733 | 60/223,416 | 08/04/2000 |
| SEQ ID NO:4734 | 60/223,416 | 08/04/2000 |
| SEQ ID NO:4735 | 60/223,416 | 08/04/2000 |
| SEQ ID NO:4736 | 60/223,416 | 08/04/2000 |
| SEQ ID NO:4737 | 60/223,416 | 08/04/2000 |
| SEQ ID NO:4738 | 60/223,416 | 08/04/2000 |
| SEQ ID NO:4739 | 60/223,416 | 08/04/2000 |
| SEQ ID NO:4740 | 60/223,416 | 08/04/2000 |
| SEQ ID NO:4741 | 60/223,416 | 08/04/2000 |
| SEQ ID NO:4742 | 60/223,416 | 08/04/2000 |
| SEQ ID NO:4743 | 60/223,416 | 08/04/2000 |
| SEQ ID NO:4744 | 60/223,416 | 08/04/2000 |
| SEQ ID NO:4745 | 60/223,416 | 08/04/2000 |
| SEQ ID NO:4746 | 60/223,416 | 08/04/2000 |
| SEQ ID NO:4747 | 60/223,416 | 08/04/2000 |
| SEQ ID NO:4748 | 60/223,416 | 08/04/2000 |
| SEQ ID NO:4749 | 60/223,416 | 08/04/2000 |
| SEQ ID NO:4750 | 60/223,416 | 08/04/2000 |
| SEQ ID NO:4751 | 60/223,416 | 08/04/2000 |
| SEQ ID NO:4752 | 60/223,416 | 08/04/2000 |
| SEQ ID NO:4753 | 60/223,416 | 08/04/2000 |
| SEQ ID NO:4754 | 60/223,416 | 08/04/2000 |
| SEQ ID NO:4755 | 60/223,416 | 08/04/2000 |
| SEQ ID NO:4756 | 60/223,416 | 08/04/2000 |
| SEQ ID NO:4757 | 60/223,416 | 08/04/2000 |
| SEQ ID NO:4758 | 60/223,416 | 08/04/2000 |
| SEQ ID NO:4759 | 60/223,416 | 08/04/2000 |
| SEQ ID NO:4760 | 60/223,416 | 08/04/2000 |
| SEQ ID NO:4761 | 60/223,416 | 08/04/2000 |
| SEQ ID NO:4762 | 60/223,416 | 08/04/2000 |
| SEQ ID NO:4763 | 60/223,416 | 08/04/2000 |
| SEQ ID NO:4764 | 60/223,416 | 08/04/2000 |
| SEQ ID NO:4765 | 60/223,416 | 08/04/2000 |
| SEQ ID NO:4766 | 60/223,416 | 08/04/2000 |
| SEQ ID NO:4767 | 60/223,416 | 08/04/2000 |
| SEQ ID NO:4768 | 60/223,416 | 08/04/2000 |
| SEQ ID NO:4769 | 60/223,416 | 08/04/2000 |
| SEQ ID NO:4770 | 60/223,416 | 08/04/2000 |
| SEQ ID NO:4771 | 60/223,416 | 08/04/2000 |
| SEQ ID NO:4772 | 60/223,416 | 08/04/2000 |
| SEQ ID NO:4773 | 60/223,416 | 08/04/2000 |
| SEQ ID NO:4774 | 60/223,416 | 08/04/2000 |
| SEQ ID NO:4775 | 60/223,416 | 08/04/2000 |
| SEQ ID NO:4776 | 60/223,416 | 08/04/2000 |
| SEQ ID NO:4777 | 60/223,416 | 08/04/2000 |
| SEQ ID NO:4778 | 60/223,416 | 08/04/2000 |
| SEQ ID NO:4779 | 60/223,416 | 08/04/2000 |
| SEQ ID NO:4780 | 60/223,416 | 08/04/2000 |
| SEQ ID NO:4781 | 60/223,416 | 08/04/2000 |
| SEQ ID NO:4782 | 60/223,416 | 08/04/2000 |
| SEQ ID NO:4783 | 60/223,416 | 08/04/2000 |
| SEQ ID NO:4784 | 60/223,416 | 08/04/2000 |
| SEQ ID NO:4785 | 60/223,416 | 08/04/2000 |
| SEQ ID NO:4786 | 60/223,416 | 08/04/2000 |
| SEQ ID NO:4787 | 60/223,416 | 08/04/2000 |
| SEQ ID NO:4788 | 60/223,416 | 08/04/2000 |
| SEQ ID NO:4789 | 60/223,416 | 08/04/2000 |
| SEQ ID NO:4790 | 60/223,416 | 08/04/2000 |
| SEQ ID NO:4791 | 60/223,416 | 08/04/2000 |
| SEQ ID NO:4792 | 60/223,416 | 08/04/2000 |
| SEQ ID NO:4793 | 60/223,416 | 08/04/2000 |
| SEQ ID NO:4794 | 60/223,416 | 08/04/2000 |
| SEQ ID NO:4795 | 60/223,416 | 08/04/2000 |
| SEQ ID NO:4796 | 60/223,416 | 08/04/2000 |
| SEQ ID NO:4797 | 60/223,416 | 08/04/2000 |
| SEQ ID NO:4798 | 60/223,416 | 08/04/2000 |
| SEQ ID NO:4799 | 60/223,416 | 08/04/2000 |
| SEQ ID NO:4800 | 60/223,416 | 08/04/2000 |
| SEQ ID NO:4801 | 60/223,416 | 08/04/2000 |
| SEQ ID NO:4802 | 60/223,416 | 08/04/2000 |
| SEQ ID NO:4803 | 60/223,416 | 08/04/2000 |
| SEQ ID NO:4804 | 60/223,416 | 08/04/2000 |
| SEQ ID NO:4805 | 60/223,416 | 08/04/2000 |
| SEQ ID NO:4806 | 60/223,416 | 08/04/2000 |
| SEQ ID NO:4807 | 60/223,416 | 08/04/2000 |
| SEQ ID NO:4808 | 60/223,416 | 08/04/2000 |
| SEQ ID NO:4809 | 60/223,416 | 08/04/2000 |
| SEQ ID NO:4810 | 60/223,416 | 08/04/2000 |
| SEQ ID NO:4811 | 60/223,416 | 08/04/2000 |
| SEQ ID NO:4812 | 60/223,416 | 08/04/2000 |
| SEQ ID NO:4813 | 60/223,416 | 08/04/2000 |
| SEQ ID NO:4814 | 60/223,416 | 08/04/2000 |
| SEQ ID NO:4815 | 60/223,416 | 08/04/2000 |
| SEQ ID NO:4816 | 60/223,416 | 08/04/2000 |
| SEQ ID NO:4817 | 60/223,416 | 08/04/2000 |
| SEQ ID NO:4818 | 60/223,416 | 08/04/2000 |
| SEQ ID NO:4819 | 60/223,416 | 08/04/2000 |
| SEQ ID NO:4820 | 60/223,416 | 08/04/2000 |
| SEQ ID NO:4821 | 60/223,416 | 08/04/2000 |
| SEQ ID NO:4822 | 60/223,416 | 08/04/2000 |
| SEQ ID NO:4823 | 60/223,416 | 08/04/2000 |
| SEQ ID NO:4824 | 60/223,416 | 08/04/2000 |
| SEQ ID NO:4825 | 60/223,416 | 08/04/2000 |
| SEQ ID NO:4826 | 60/223,416 | 08/04/2000 |
| SEQ ID NO:4827 | 60/223,416 | 08/04/2000 |
| SEQ ID NO:4828 | 60/223,416 | 08/04/2000 |
| SEQ ID NO:4829 | 60/223,416 | 08/04/2000 |
| SEQ ID NO:4830 | 60/223,416 | 08/04/2000 |
| SEQ ID NO:4831 | 60/223,416 | 08/04/2000 |
| SEQ ID NO:4832 | 60/223,416 | 08/04/2000 |
| SEQ ID NO:4833 | 60/223,416 | 08/04/2000 |
| SEQ ID NO:4834 | 60/223,416 | 08/04/2000 |
| SEQ ID NO:4835 | 60/223,416 | 08/04/2000 |
| SEQ ID NO:4836 | 60/223,416 | 08/04/2000 |
| SEQ ID NO:4837 | 60/223,416 | 08/04/2000 |
| SEQ ID NO:4838 | 60/223,416 | 08/04/2000 |
| SEQ ID NO:4839 | 60/223,416 | 08/04/2000 |
| SEQ ID NO:4840 | 60/223,416 | 08/04/2000 |
| SEQ ID NO:4841 | 60/223,416 | 08/04/2000 |
| SEQ ID NO:4842 | 60/223,416 | 08/04/2000 |
| SEQ ID NO:4843 | 60/223,416 | 08/04/2000 |
| SEQ ID NO:4844 | 60/223,416 | 08/04/2000 |
| SEQ ID NO:4845 | 60/223,416 | 08/04/2000 |
| SEQ ID NO:4846 | 60/223,416 | 08/04/2000 |
| SEQ ID NO:4847 | 60/223,416 | 08/04/2000 |
| SEQ ID NO:4848 | 60/223,416 | 08/04/2000 |
| SEQ ID NO:4849 | 60/223,416 | 08/04/2000 |
| SEQ ID NO:4850 | 60/223,416 | 08/04/2000 |
| SEQ ID NO:4851 | 60/223,416 | 08/04/2000 |
| SEQ ID NO:4852 | 60/223,416 | 08/04/2000 |
| SEQ ID NO:4853 | 60/223,416 | 08/04/2000 |
| SEQ ID NO:4854 | 60/223,416 | 08/04/2000 |
| SEQ ID NO:4855 | 60/223,416 | 08/04/2000 |
| SEQ ID NO:4856 | 60/223,416 | 08/04/2000 |
| SEQ ID NO:4857 | 60/223,416 | 08/04/2000 |
| SEQ ID NO:4858 | 60/223,416 | 08/04/2000 |
| SEQ ID NO:4859 | 60/223,416 | 08/04/2000 |
| SEQ ID NO:4860 | 60/223,416 | 08/04/2000 |
| SEQ ID NO:4861 | 60/223,416 | 08/04/2000 |
| SEQ ID NO:4862 | 60/223,416 | 08/04/2000 |
| SEQ ID NO:4863 | 60/223,416 | 08/04/2000 |
| SEQ ID NO:4864 | 60/223,416 | 08/04/2000 |
| SEQ ID NO:4865 | 60/223,416 | 08/04/2000 |
| SEQ ID NO:4866 | 60/223,416 | 08/04/2000 |
| SEQ ID NO:4867 | 60/223,416 | 08/04/2000 |
| SEQ ID NO:4868 | 60/223,416 | 08/04/2000 |
| SEQ ID NO:4869 | 60/223,416 | 08/04/2000 |
| SEQ ID NO:4870 | 60/223,416 | 08/04/2000 |
| SEQ ID NO:4871 | 60/223,416 | 08/04/2000 |
| SEQ ID NO:4872 | 60/223,416 | 08/04/2000 |
| SEQ ID NO:4873 | 60/223,416 | 08/04/2000 |
| SEQ ID NO:4874 | 60/223,416 | 08/04/2000 |
| SEQ ID NO:4875 | 60/223,416 | 08/04/2000 |
| SEQ ID NO:4876 | 60/223,416 | 08/04/2000 |
| SEQ ID NO:4877 | 60/223,416 | 08/04/2000 |
| SEQ ID NO:4878 | 60/223,416 | 08/04/2000 |
| SEQ ID NO:4879 | 60/223,416 | 08/04/2000 |
| SEQ ID NO:4880 | 60/223,416 | 08/04/2000 |
| SEQ ID NO:4881 | 60/223,416 | 08/04/2000 |
| SEQ ID NO:4882 | 60/223,416 | 08/04/2000 |
| SEQ ID NO:4883 | 60/223,416 | 08/04/2000 |
| SEQ ID NO:4884 | 60/223,416 | 08/04/2000 |
| SEQ ID NO:4885 | 60/223,416 | 08/04/2000 |
| SEQ ID NO:4886 | 60/223,416 | 08/04/2000 |
| SEQ ID NO:4887 | 60/223,416 | 08/04/2000 |
| SEQ ID NO:4888 | 60/223,416 | 08/04/2000 |
| SEQ ID NO:4889 | 60/223,416 | 08/04/2000 |
| SEQ ID NO:4890 | 60/223,416 | 08/04/2000 |
| SEQ ID NO:4891 | 60/223,416 | 08/04/2000 |
| SEQ ID NO:4892 | 60/223,416 | 08/04/2000 |
| SEQ ID NO:4893 | 60/223,416 | 08/04/2000 |
| SEQ ID NO:4894 | 60/223,416 | 08/04/2000 |
| SEQ ID NO:4895 | 60/223,416 | 08/04/2000 |
| SEQ ID NO:4896 | 60/223,416 | 08/04/2000 |
| SEQ ID NO:4897 | 60/223,416 | 08/04/2000 |
| SEQ ID NO:4898 | 60/223,416 | 08/04/2000 |
| SEQ ID NO:4899 | 60/223,416 | 08/04/2000 |
| SEQ ID NO:4900 | 60/223,416 | 08/04/2000 |
| SEQ ID NO:4901 | 60/223,416 | 08/04/2000 |
| SEQ ID NO:4902 | 60/223,416 | 08/04/2000 |
| SEQ ID NO:4903 | 60/223,416 | 08/04/2000 |
| SEQ ID NO:4904 | 60/223,416 | 08/04/2000 |
| SEQ ID NO:4905 | 60/223,416 | 08/04/2000 |
| SEQ ID NO:4906 | 60/223,416 | 08/04/2000 |
| SEQ ID NO:4907 | 60/223,416 | 08/04/2000 |
| SEQ ID NO:4908 | 60/223,416 | 08/04/2000 |
| SEQ ID NO:4909 | 60/223,416 | 08/04/2000 |
| SEQ ID NO:4910 | 60/223,416 | 08/04/2000 |
| SEQ ID NO:4911 | 60/223,416 | 08/04/2000 |
| SEQ ID NO:4912 | 60/223,416 | 08/04/2000 |
| SEQ ID NO:4913 | 60/223,416 | 08/04/2000 |
| SEQ ID NO:4914 | 60/223,416 | 08/04/2000 |
| SEQ ID NO:4915 | 60/223,416 | 08/04/2000 |
| SEQ ID NO:4916 | 60/223,416 | 08/04/2000 |
| SEQ ID NO:4917 | 60/223,416 | 08/04/2000 |
| SEQ ID NO:4918 | 60/223,416 | 08/04/2000 |
| SEQ ID NO:4919 | 60/223,416 | 08/04/2000 |
| SEQ ID NO:4920 | 60/223,416 | 08/04/2000 |
| SEQ ID NO:4921 | 60/223,416 | 08/04/2000 |
| SEQ ID NO:4922 | 60/223,416 | 08/04/2000 |
| SEQ ID NO:4923 | 60/223,416 | 08/04/2000 |
| SEQ ID NO:4924 | 60/223,416 | 08/04/2000 |
| SEQ ID NO:4925 | 60/223,416 | 08/04/2000 |
| SEQ ID NO:4926 | 60/223,416 | 08/04/2000 |
| SEQ ID NO:4927 | 60/223,416 | 08/04/2000 |
| SEQ ID NO:4928 | 60/223,416 | 08/04/2000 |
| SEQ ID NO:4929 | 60/223,416 | 08/04/2000 |
| SEQ ID NO:4930 | 60/223,416 | 08/04/2000 |
| SEQ ID NO:4931 | 60/223,416 | 08/04/2000 |
| SEQ ID NO:4932 | 60/223,416 | 08/04/2000 |
| SEQ ID NO:4933 | 60/223,416 | 08/04/2000 |
| SEQ ID NO:4934 | 60/223,416 | 08/04/2000 |
| SEQ ID NO:4935 | 60/223,416 | 08/04/2000 |
| SEQ ID NO:4936 | 60/223,416 | 08/04/2000 |
| SEQ ID NO:4937 | 60/223,416 | 08/04/2000 |
| SEQ ID NO:4938 | 60/223,416 | 08/04/2000 |
| SEQ ID NO:4939 | 60/223,416 | 08/04/2000 |
| SEQ ID NO:4940 | 60/223,416 | 08/04/2000 |
| SEQ ID NO:4941 | 60/223,416 | 08/04/2000 |
| SEQ ID NO:4942 | 60/223,416 | 08/04/2000 |
| SEQ ID NO:4943 | 60/223,416 | 08/04/2000 |
| SEQ ID NO:4944 | 60/223,416 | 08/04/2000 |
| SEQ ID NO:4945 | 60/223,416 | 08/04/2000 |
| SEQ ID NO:4946 | 60/223,416 | 08/04/2000 |
| SEQ ID NO:4947 | 60/223,416 | 08/04/2000 |
| SEQ ID NO:4948 | 60/223,416 | 08/04/2000 |
| SEQ ID NO:4949 | 60/223,416 | 08/04/2000 |
| SEQ ID NO:4950 | 60/223,416 | 08/04/2000 |
| SEQ ID NO:4951 | 60/223,416 | 08/04/2000 |
| SEQ ID NO:4952 | 60/223,416 | 08/04/2000 |
| SEQ ID NO:4953 | 60/223,416 | 08/04/2000 |
| SEQ ID NO:4954 | 60/223,416 | 08/04/2000 |
| SEQ ID NO:4955 | 60/223,416 | 08/04/2000 |
| SEQ ID NO:4956 | 60/223,416 | 08/04/2000 |
| SEQ ID NO:4957 | 60/223,416 | 08/04/2000 |
| SEQ ID NO:4958 | 60/223,416 | 08/04/2000 |
| SEQ ID NO:4959 | 60/223,416 | 08/04/2000 |
| SEQ ID NO:4960 | 60/223,416 | 08/04/2000 |
| SEQ ID NO:4961 | 60/223,416 | 08/04/2000 |
| SEQ ID NO:4962 | 60/223,416 | 08/04/2000 |
| SEQ ID NO:4963 | 60/223,416 | 08/04/2000 |
| SEQ ID NO:4964 | 60/223,416 | 08/04/2000 |
| SEQ ID NO:4965 | 60/223,416 | 08/04/2000 |
| SEQ ID NO:4966 | 60/223,416 | 08/04/2000 |
| SEQ ID NO:4967 | 60/223,416 | 08/04/2000 |
| SEQ ID NO:4968 | 60/223,416 | 08/04/2000 |
| SEQ ID NO:4969 | 60/223,416 | 08/04/2000 |
| SEQ ID NO:4970 | 60/223,416 | 08/04/2000 |
| SEQ ID NO:4971 | 60/223,416 | 08/04/2000 |
| SEQ ID NO:4972 | 60/223,416 | 08/04/2000 |
| SEQ ID NO:4973 | 60/223,416 | 08/04/2000 |
| SEQ ID NO:4974 | 60/223,416 | 08/04/2000 |
| SEQ ID NO:4975 | 60/223,416 | 08/04/2000 |
| SEQ ID NO:4976 | 60/223,416 | 08/04/2000 |
| SEQ ID NO:4977 | 60/223,416 | 08/04/2000 |
| SEQ ID NO:4978 | 60/223,416 | 08/04/2000 |
| SEQ ID NO:4979 | 60/223,416 | 08/04/2000 |
| SEQ ID NO:4980 | 60/223,416 | 08/04/2000 |
| SEQ ID NO:4981 | 60/223,416 | 08/04/2000 |
| SEQ ID NO:4982 | 60/223,416 | 08/04/2000 |
| SEQ ID NO:4983 | 60/223,416 | 08/04/2000 |
| SEQ ID NO:4984 | 60/223,416 | 08/04/2000 |
| SEQ ID NO:4985 | 60/223,416 | 08/04/2000 |
| SEQ ID NO:4986 | 60/223,416 | 08/04/2000 |
| SEQ ID NO:4987 | 60/223,416 | 08/04/2000 |
| SEQ ID NO:4988 | 60/223,416 | 08/04/2000 |
| SEQ ID NO:4989 | 60/223,416 | 08/04/2000 |
| SEQ ID NO:4990 | 60/223,416 | 08/04/2000 |
| SEQ ID NO:4991 | 60/223,416 | 08/04/2000 |
| SEQ ID NO:4992 | 60/223,416 | 08/04/2000 |
| SEQ ID NO:4993 | 60/223,416 | 08/04/2000 |
| SEQ ID NO:4994 | 60/223,416 | 08/04/2000 |
| SEQ ID NO:4995 | 60/223,416 | 08/04/2000 |
| SEQ ID NO:4996 | 60/223,416 | 08/04/2000 |
| SEQ ID NO:4997 | 60/223,416 | 08/04/2000 |
| SEQ ID NO:4998 | 60/223,416 | 08/04/2000 |
| SEQ ID NO:4999 | 60/223,416 | 08/04/2000 |
| SEQ ID NO:5000 | 60/223,416 | 08/04/2000 |
| SEQ ID NO:5001 | 60/223,416 | 08/04/2000 |
| SEQ ID NO:5002 | 60/223,416 | 08/04/2000 |
| SEQ ID NO:5003 | 60/223,416 | 08/04/2000 |
| SEQ ID NO:5004 | 60/223,416 | 08/04/2000 |
| SEQ ID NO:5005 | 60/223,416 | 08/04/2000 |
| SEQ ID NO:5006 | 60/223,416 | 08/04/2000 |
| SEQ ID NO:5007 | 60/223,416 | 08/04/2000 |
| SEQ ID NO:5008 | 60/223,416 | 08/04/2000 |
| SEQ ID NO:5009 | 60/223,416 | 08/04/2000 |
| SEQ ID NO:5010 | 60/223,416 | 08/04/2000 |
| SEQ ID NO:5011 | 60/223,416 | 08/04/2000 |
| SEQ ID NO:5012 | 60/223,416 | 08/04/2000 |
| SEQ ID NO:5013 | 60/223,416 | 08/04/2000 |
| SEQ ID NO:5014 | 60/223,416 | 08/04/2000 |
| SEQ ID NO:5015 | 60/223,416 | 08/04/2000 |
| SEQ ID NO:5016 | 60/223,416 | 08/04/2000 |
| SEQ ID NO:5017 | 60/223,416 | 08/04/2000 |
| SEQ ID NO:5018 | 60/223,416 | 08/04/2000 |
| SEQ ID NO:5019 | 60/223,416 | 08/04/2000 |
| SEQ ID NO:5020 | 60/223,416 | 08/04/2000 |
| SEQ ID NO:5021 | 60/223,416 | 08/04/2000 |
| SEQ ID NO:5022 | 60/223,416 | 08/04/2000 |
| SEQ ID NO:5023 | 60/223,416 | 08/04/2000 |
| SEQ ID NO:5024 | 60/223,416 | 08/04/2000 |
| SEQ ID NO:5025 | 60/223,416 | 08/04/2000 |
| SEQ ID NO:5026 | 60/223,416 | 08/04/2000 |
| SEQ ID NO:5027 | 60/223,416 | 08/04/2000 |
| SEQ ID NO:5028 | 60/223,416 | 08/04/2000 |
| SEQ ID NO:5029 | 60/223,416 | 08/04/2000 |
| SEQ ID NO:5030 | 60/223,416 | 08/04/2000 |
| SEQ ID NO:5031 | 60/223,416 | 08/04/2000 |
| SEQ ID NO:5032 | 60/223,416 | 08/04/2000 |
| SEQ ID NO:5033 | 60/223,416 | 08/04/2000 |
| SEQ ID NO:5034 | 60/223,416 | 08/04/2000 |
| SEQ ID NO:5035 | 60/223,416 | 08/04/2000 |
| SEQ ID NO:5036 | 60/223,416 | 08/04/2000 |
| SEQ ID NO:5037 | 60/223,416 | 08/04/2000 |
| SEQ ID NO:5038 | 60/223,416 | 08/04/2000 |
| SEQ ID NO:5039 | 60/223,416 | 08/04/2000 |
| SEQ ID NO:5040 | 60/223,416 | 08/04/2000 |
| SEQ ID NO:5041 | 60/223,416 | 08/04/2000 |
| SEQ ID NO:5042 | 60/223,416 | 08/04/2000 |
| SEQ ID NO:5043 | 60/223,416 | 08/04/2000 |
| SEQ ID NO:5044 | 60/223,416 | 08/04/2000 |
| SEQ ID NO:5045 | 60/223,416 | 08/04/2000 |
| SEQ ID NO:5046 | 60/223,416 | 08/04/2000 |
| SEQ ID NO:5047 | 60/223,416 | 08/04/2000 |
| SEQ ID NO:5048 | 60/223,416 | 08/04/2000 |
| SEQ ID NO:5049 | 60/223,416 | 08/04/2000 |
| SEQ ID NO:5050 | 60/223,416 | 08/04/2000 |
| SEQ ID NO:5051 | 60/223,416 | 08/04/2000 |
| SEQ ID NO:5052 | 60/223,416 | 08/04/2000 |
| SEQ ID NO:5053 | 60/223,416 | 08/04/2000 |
| SEQ ID NO:5054 | 60/223,416 | 08/04/2000 |
| SEQ ID NO:5055 | 60/223,416 | 08/04/2000 |
| SEQ ID NO:5056 | 60/223,416 | 08/04/2000 |
| SEQ ID NO:5057 | 60/223,416 | 08/04/2000 |
| SEQ ID NO:5058 | 60/223,416 | 08/04/2000 |
| SEQ ID NO:5059 | 60/223,416 | 08/04/2000 |
| SEQ ID NO:5060 | 60/223,416 | 08/04/2000 |
| SEQ ID NO:5061 | 60/223,416 | 08/04/2000 |
| SEQ ID NO:5062 | 60/223,416 | 08/04/2000 |
| SEQ ID NO:5063 | 60/223,416 | 08/04/2000 |
| SEQ ID NO:5064 | 60/223,416 | 08/04/2000 |
| SEQ ID NO:5065 | 60/223,416 | 08/04/2000 |
| SEQ ID NO:5066 | 60/223,416 | 08/04/2000 |
| SEQ ID NO:5067 | 60/223,416 | 08/04/2000 |
| SEQ ID NO:5068 | 60/223,416 | 08/04/2000 |
| SEQ ID NO:5069 | 60/223,416 | 08/04/2000 |
| SEQ ID NO:5070 | 60/223,416 | 08/04/2000 |
| SEQ ID NO:5071 | 60/223,416 | 08/04/2000 |
| SEQ ID NO:5072 | 60/223,416 | 08/04/2000 |
| SEQ ID NO:5080 | 60/223,416 | 08/04/2000 |
| SEQ ID NO:5081 | 60/223,416 | 08/04/2000 |
| SEQ ID NO:5082 | 60/223,416 | 08/04/2000 |
| SEQ ID NO:5083 | 60/223,416 | 08/04/2000 |
| SEQ ID NO:5084 | 60/223,416 | 08/04/2000 |
| SEQ ID NO:5085 | 60/223,416 | 08/04/2000 |
| SEQ ID NO:5086 | 60/223,416 | 08/04/2000 |
| SEQ ID NO:5087 | 60/223,416 | 08/04/2000 |
| SEQ ID NO:5088 | 60/223,416 | 08/04/2000 |
| SEQ ID NO:5089 | 60/223,416 | 08/04/2000 |
| SEQ ID NO:5090 | 60/223,416 | 08/04/2000 |
| SEQ ID NO:5091 | 60/223,416 | 08/04/2000 |
| SEQ ID NO:5092 | 60/223,416 | 08/04/2000 |
| SEQ ID NO:5093 | 60/223,416 | 08/04/2000 |
| SEQ ID NO:5094 | 60/223,416 | 08/04/2000 |
| SEQ ID NO:5095 | 60/223,416 | 08/04/2000 |
| SEQ ID NO:5096 | 60/223,416 | 08/04/2000 |
| SEQ ID NO:5097 | 60/223,416 | 08/04/2000 |
| SEQ ID NO:5098 | 60/223,416 | 08/04/2000 |
| SEQ ID NO:5099 | 60/223,416 | 08/04/2000 |
| SEQ ID NO:5100 | 60/223,416 | 08/04/2000 |
| SEQ ID NO:5101 | 60/223,416 | 08/04/2000 |
| SEQ ID NO:5102 | 60/223,416 | 08/04/2000 |
| SEQ ID NO:5103 | 60/223,416 | 08/04/2000 |
| SEQ ID NO:5104 | 60/223,416 | 08/04/2000 |
| SEQ ID NO:5105 | 60/223,416 | 08/04/2000 |
| SEQ ID NO:5106 | 60/223,416 | 08/04/2000 |
| SEQ ID NO:5107 | 60/223,416 | 08/04/2000 |
| SEQ ID NO:5108 | 60/223,416 | 08/04/2000 |
| SEQ ID NO:5109 | 60/223,416 | 08/04/2000 |
| SEQ ID NO:5110 | 60/223,416 | 08/04/2000 |
| SEQ ID NO:5111 | 60/223,416 | 08/04/2000 |
| SEQ ID NO:5112 | 60/223,416 | 08/04/2000 |
| SEQ ID NO:5113 | 60/223,416 | 08/04/2000 |
| SEQ ID NO:5114 | 60/223,416 | 08/04/2000 |
| SEQ ID NO:5115 | 60/223,416 | 08/04/2000 |
| SEQ ID NO:5116 | 60/223,416 | 08/04/2000 |
| SEQ ID NO:5117 | 60/223,416 | 08/04/2000 |
| SEQ ID NO:5118 | 60/223,416 | 08/04/2000 |
| SEQ ID NO:5119 | 60/223,416 | 08/04/2000 |
| SEQ ID NO:5120 | 60/223,416 | 08/04/2000 |
| SEQ ID NO:5121 | 60/223,416 | 08/04/2000 |
| SEQ ID NO:5122 | 60/223,416 | 08/04/2000 |
| SEQ ID NO:5123 | 60/223,416 | 08/04/2000 |
| SEQ ID NO:5124 | 60/223,416 | 08/04/2000 |
| SEQ ID NO:5125 | 60/223,416 | 08/04/2000 |
| SEQ ID NO:5126 | 60/223,416 | 08/04/2000 |
| SEQ ID NO:5127 | 60/223,416 | 08/04/2000 |
| SEQ ID NO:5128 | 60/223,416 | 08/04/2000 |
| SEQ ID NO:5129 | 60/223,416 | 08/04/2000 |
| SEQ ID NO:5130 | 60/223,416 | 08/04/2000 |
| SEQ ID NO:5131 | 60/223,416 | 08/04/2000 |
| SEQ ID NO:5132 | 60/223,416 | 08/04/2000 |
| SEQ ID NO:5133 | 60/223,416 | 08/04/2000 |
| SEQ ID NO:5134 | 60/223,416 | 08/04/2000 |
| SEQ ID NO:5135 | 60/223,416 | 08/04/2000 |
| SEQ ID NO:5136 | 60/223,416 | 08/04/2000 |
| SEQ ID NO:5137 | 60/223,416 | 08/04/2000 |
| SEQ ID NO:5138 | 60/223,416 | 08/04/2000 |
| SEQ ID NO:5139 | 60/223,416 | 08/04/2000 |
| SEQ ID NO:5140 | 60/223,416 | 08/04/2000 |
| SEQ ID NO:5141 | 60/223,416 | 08/04/2000 |
| SEQ ID NO:5142 | 60/223,416 | 08/04/2000 |
| SEQ ID NO:5143 | 60/223,416 | 08/04/2000 |
| SEQ ID NO:5144 | 60/223,416 | 08/04/2000 |
| SEQ ID NO:5145 | 60/223,416 | 08/04/2000 |
| SEQ ID NO:5146 | 60/223,416 | 08/04/2000 |
| SEQ ID NO:5147 | 60/223,416 | 08/04/2000 |
| SEQ ID NO:5148 | 60/223,416 | 08/04/2000 |
| SEQ ID NO:5149 | 60/223,416 | 08/04/2000 |
| SEQ ID NO:5150 | 60/223,416 | 08/04/2000 |
| SEQ ID NO:5151 | 60/223,416 | 08/04/2000 |
| SEQ ID NO:5152 | 60/223,416 | 08/04/2000 |
| SEQ ID NO:5153 | 60/223,416 | 08/04/2000 |
| SEQ ID NO:5154 | 60/223,416 | 08/04/2000 |
| SEQ ID NO:5155 | 60/223,416 | 08/04/2000 |
| SEQ ID NO:5156 | 60/223,416 | 08/04/2000 |
| SEQ ID NO:5157 | 60/223,416 | 08/04/2000 |
| SEQ ID NO:5158 | 60/223,416 | 08/04/2000 |
| SEQ ID NO:5159 | 60/223,416 | 08/04/2000 |
| SEQ ID NO:5160 | 60/223,416 | 08/04/2000 |
| SEQ ID NO:5161 | 60/223,416 | 08/04/2000 |
| SEQ ID NO:5162 | 60/223,416 | 08/04/2000 |
| SEQ ID NO:5163 | 60/223,416 | 08/04/2000 |
| SEQ ID NO:5164 | 60/223,416 | 08/04/2000 |
| SEQ ID NO:5165 | 60/223,416 | 08/04/2000 |
| SEQ ID NO:5166 | 60/223,416 | 08/04/2000 |
| SEQ ID NO:5167 | 60/223,416 | 08/04/2000 |
| SEQ ID NO:5168 | 60/223,416 | 08/04/2000 |
| SEQ ID NO:5169 | 60/223,416 | 08/04/2000 |
| SEQ ID NO:5170 | 60/223,416 | 08/04/2000 |
| SEQ ID NO:5171 | 60/223,416 | 08/04/2000 |
| SEQ ID NO:5172 | 60/223,416 | 08/04/2000 |
| SEQ ID NO:5173 | 60/223,416 | 08/04/2000 |
| SEQ ID NO:5174 | 60/223,416 | 08/04/2000 |
| SEQ ID NO:5175 | 60/223,416 | 08/04/2000 |
| SEQ ID NO:5176 | 60/223,416 | 08/04/2000 |
| SEQ ID NO:5177 | 60/223,416 | 08/04/2000 |
| SEQ ID NO:5178 | 60/223,416 | 08/04/2000 |
| SEQ ID NO:5179 | 60/223,416 | 08/04/2000 |
| SEQ ID NO:5180 | 60/223,416 | 08/04/2000 |
| SEQ ID NO:5181 | 60/223,416 | 08/04/2000 |
| SEQ ID NO:5182 | 60/223,416 | 08/04/2000 |
| SEQ ID NO:5183 | 60/223,416 | 08/04/2000 |
| SEQ ID NO:5184 | 60/223,416 | 08/04/2000 |
| SEQ ID NO:5185 | 60/223,416 | 08/04/2000 |
| SEQ ID NO:5186 | 60/223,416 | 08/04/2000 |
| SEQ ID NO:5187 | 60/223,416 | 08/04/2000 |
| SEQ ID NO:5188 | 60/223,416 | 08/04/2000 |
| SEQ ID NO:5189 | 60/223,416 | 08/04/2000 |
| SEQ ID NO:5190 | 60/223,416 | 08/04/2000 |
| SEQ ID NO:5191 | 60/223,416 | 08/04/2000 |
| SEQ ID NO:5192 | 60/223,416 | 08/04/2000 |
| SEQ ID NO:5193 | 60/223,416 | 08/04/2000 |
| SEQ ID NO:5194 | 60/223,416 | 08/04/2000 |
| SEQ ID NO:5195 | 60/223,416 | 08/04/2000 |
| SEQ ID NO:5196 | 60/223,416 | 08/04/2000 |
| SEQ ID NO:5197 | 60/223,416 | 08/04/2000 |
| SEQ ID NO:5198 | 60/223,416 | 08/04/2000 |
| SEQ ID NO:5199 | 60/223,416 | 08/04/2000 |
| SEQ ID NO:5200 | 60/223,416 | 08/04/2000 |
| SEQ ID NO:5201 | 60/223,416 | 08/04/2000 |
| SEQ ID NO:5202 | 60/223,416 | 08/04/2000 |
| SEQ ID NO:5203 | 60/223,416 | 08/04/2000 |
| SEQ ID NO:5204 | 60/223,416 | 08/04/2000 |
| SEQ ID NO:5205 | 60/223,416 | 08/04/2000 |
| SEQ ID NO:5206 | 60/223,416 | 08/04/2000 |
| SEQ ID NO:5207 | 60/223,416 | 08/04/2000 |
| SEQ ID NO:5208 | 60/223,416 | 08/04/2000 |
| SEQ ID NO:5209 | 60/223,416 | 08/04/2000 |
| SEQ ID NO:5210 | 60/223,416 | 08/04/2000 |
| SEQ ID NO:5211 | 60/223,416 | 08/04/2000 |
| SEQ ID NO:5212 | 60/223,416 | 08/04/2000 |
| SEQ ID NO:5213 | 60/223,416 | 08/04/2000 |
| SEQ ID NO:5214 | 60/223,416 | 08/04/2000 |
| SEQ ID NO:5215 | 60/223,416 | 08/04/2000 |
| SEQ ID NO:5216 | 60/223,416 | 08/04/2000 |
| SEQ ID NO:5217 | 60/223,416 | 08/04/2000 |
| SEQ ID NO:5218 | 60/223,416 | 08/04/2000 |
| SEQ ID NO:5219 | 60/223,416 | 08/04/2000 |
| SEQ ID NO:5220 | 60/223,416 | 08/04/2000 |
| SEQ ID NO:5221 | 60/223,416 | 08/04/2000 |
| SEQ ID NO:5222 | 60/223,416 | 08/04/2000 |
| SEQ ID NO:5223 | 60/223,416 | 08/04/2000 |
| SEQ ID NO:5224 | 60/223,416 | 08/04/2000 |
| SEQ ID NO:5225 | 60/223,416 | 08/04/2000 |
| SEQ ID NO:5226 | 60/223,416 | 08/04/2000 |
| SEQ ID NO:5227 | 60/223,416 | 08/04/2000 |
| SEQ ID NO:5228 | 60/223,416 | 08/04/2000 |
| SEQ ID NO:5229 | 60/223,416 | 08/04/2000 |
| SEQ ID NO:5230 | 60/223,416 | 08/04/2000 |
| SEQ ID NO:5231 | 60/223,416 | 08/04/2000 |
| SEQ ID NO:5232 | 60/223,416 | 08/04/2000 |
| SEQ ID NO:5233 | 60/223,416 | 08/04/2000 |
| SEQ ID NO:5234 | 60/223,416 | 08/04/2000 |
| SEQ ID NO:5235 | 60/223,416 | 08/04/2000 |
| SEQ ID NO:5236 | 60/223,416 | 08/04/2000 |
| SEQ ID NO:5237 | 60/223,416 | 08/04/2000 |
| SEQ ID NO:5238 | 60/223,416 | 08/04/2000 |
| SEQ ID NO:5239 | 60/223,416 | 08/04/2000 |
| SEQ ID NO:5240 | 60/223,416 | 08/04/2000 |
| SEQ ID NO:5241 | 60/223,416 | 08/04/2000 |
| SEQ ID NO:5242 | 60/223,416 | 08/04/2000 |
| SEQ ID NO:5243 | 60/223,416 | 08/04/2000 |
| SEQ ID NO:5244 | 60/223,416 | 08/04/2000 |
| SEQ ID NO:5245 | 60/223,416 | 08/04/2000 |
| SEQ ID NO:5246 | 60/223,416 | 08/04/2000 |
| SEQ ID NO:5247 | 60/223,416 | 08/04/2000 |
| SEQ ID NO:5248 | 60/223,416 | 08/04/2000 |
| SEQ ID NO:5249 | 60/223,416 | 08/04/2000 |
| SEQ ID NO:5250 | 60/223,416 | 08/04/2000 |
| SEQ ID NO:5251 | 60/223,416 | 08/04/2000 |
| SEQ ID NO:5252 | 60/223,416 | 08/04/2000 |
| SEQ ID NO:5253 | 60/223,416 | 08/04/2000 |
| SEQ ID NO:5254 | 60/223,416 | 08/04/2000 |
| SEQ ID NO:5255 | 60/223,416 | 08/04/2000 |
| SEQ ID NO:5256 | 60/223,416 | 08/04/2000 |
| SEQ ID NO:5257 | 60/223,416 | 08/04/2000 |
| SEQ ID NO:5258 | 60/223,416 | 08/04/2000 |
| SEQ ID NO:5259 | 60/223,416 | 08/04/2000 |
| SEQ ID NO:5260 | 60/223,416 | 08/04/2000 |
| SEQ ID NO:5261 | 60/223,416 | 08/04/2000 |
| SEQ ID NO:5262 | 60/223,416 | 08/04/2000 |
| SEQ ID NO:5263 | 60/223,416 | 08/04/2000 |
| SEQ ID NO:5264 | 60/223,416 | 08/04/2000 |
| SEQ ID NO:5265 | 60/223,416 | 08/04/2000 |
| SEQ ID NO:5266 | 60/223,416 | 08/04/2000 |
| SEQ ID NO:5267 | 60/223,416 | 08/04/2000 |
| SEQ ID NO:5268 | 60/223,416 | 08/04/2000 |
| SEQ ID NO:5269 | 60/223,416 | 08/04/2000 |
| SEQ ID NO:5270 | 60/223,416 | 08/04/2000 |
| SEQ ID NO:5271 | 60/223,416 | 08/04/2000 |
| SEQ ID NO:5272 | 60/223,416 | 08/04/2000 |
| SEQ ID NO:5273 | 60/223,416 | 08/04/2000 |
| SEQ ID NO:5274 | 60/223,416 | 08/04/2000 |
| SEQ ID NO:5275 | 60/223,416 | 08/04/2000 |
| SEQ ID NO:5276 | 60/223,416 | 08/04/2000 |
| SEQ ID NO:5277 | 60/223,416 | 08/04/2000 |
| SEQ ID NO:5278 | 60/223,416 | 08/04/2000 |
| SEQ ID NO:5279 | 60/223,416 | 08/04/2000 |
| SEQ ID NO:5280 | 60/223,416 | 08/04/2000 |
| SEQ ID NO:5281 | 60/223,416 | 08/04/2000 |
| SEQ ID NO:5282 | 60/223,416 | 08/04/2000 |
| SEQ ID NO:5283 | 60/223,416 | 08/04/2000 |
| SEQ ID NO:5284 | 60/223,416 | 08/04/2000 |
| SEQ ID NO:5285 | 60/223,416 | 08/04/2000 |
| SEQ ID NO:5286 | 60/223,416 | 08/04/2000 |
| SEQ ID NO:5287 | 60/223,416 | 08/04/2000 |
| SEQ ID NO:5288 | 60/223,416 | 08/04/2000 |
| SEQ ID NO:5289 | 60/223,416 | 08/04/2000 |
| SEQ ID NO:5290 | 60/223,416 | 08/04/2000 |
| SEQ ID NO:5291 | 60/223,416 | 08/04/2000 |
| SEQ ID NO:5292 | 60/223,416 | 08/04/2000 |
| SEQ ID NO:5293 | 60/223,416 | 08/04/2000 |
| SEQ ID NO:5294 | 60/223,416 | 08/04/2000 |
| SEQ ID NO:5295 | 60/223,416 | 08/04/2000 |
| SEQ ID NO:5296 | 60/223,416 | 08/04/2000 |
| SEQ ID NO:5297 | 60/223,416 | 08/04/2000 |
| SEQ ID NO:5298 | 60/223,416 | 08/04/2000 |
| SEQ ID NO:5299 | 60/223,416 | 08/04/2000 |
| SEQ ID NO:5300 | 60/223,416 | 08/04/2000 |
| SEQ ID NO:5301 | 60/223,416 | 08/04/2000 |
| SEQ ID NO:5302 | 60/223,416 | 08/04/2000 |
| SEQ ID NO:5303 | 60/223,416 | 08/04/2000 |
| SEQ ID NO:5304 | 60/223,416 | 08/04/2000 |
| SEQ ID NO:5305 | 60/223,416 | 08/04/2000 |
| SEQ ID NO:5306 | 60/223,416 | 08/04/2000 |
| SEQ ID NO:5307 | 60/223,416 | 08/04/2000 |
| SEQ ID NO:5308 | 60/223,416 | 08/04/2000 |
| SEQ ID NO:5309 | 60/223,416 | 08/04/2000 |
| SEQ ID NO:5310 | 60/223,416 | 08/04/2000 |
| SEQ ID NO:5311 | 60/223,416 | 08/04/2000 |
| SEQ ID NO:5312 | 60/223,416 | 08/04/2000 |
| SEQ ID NO:5313 | 60/223,416 | 08/04/2000 |
| SEQ ID NO:5314 | 60/223,416 | 08/04/2000 |
| SEQ ID NO:5315 | 60/223,416 | 08/04/2000 |
| SEQ ID NO:5316 | 60/223,416 | 08/04/2000 |
| SEQ ID NO:5317 | 60/223,416 | 08/04/2000 |
| SEQ ID NO:5318 | 60/223,416 | 08/04/2000 |
| SEQ ID NO:5319 | 60/223,416 | 08/04/2000 |
| SEQ ID NO:5320 | 60/223,416 | 08/04/2000 |
| SEQ ID NO:5321 | 60/223,416 | 08/04/2000 |
| SEQ ID NO:5322 | 60/223,416 | 08/04/2000 |
| SEQ ID NO:5323 | 60/223,416 | 08/04/2000 |
| SEQ ID NO:5324 | 60/223,416 | 08/04/2000 |
| SEQ ID NO:5325 | 60/223,416 | 08/04/2000 |
| SEQ ID NO:5326 | 60/223,416 | 08/04/2000 |
| SEQ ID NO:5327 | 60/223,416 | 08/04/2000 |
| SEQ ID NO:5328 | 60/223,416 | 08/04/2000 |
| SEQ ID NO:5329 | 60/223,416 | 08/04/2000 |
| SEQ ID NO:5330 | 60/223,416 | 08/04/2000 |
| SEQ ID NO:5331 | 60/223,416 | 08/04/2000 |
| SEQ ID NO:5332 | 60/223,416 | 08/04/2000 |
| SEQ ID NO:5333 | 60/223,416 | 08/04/2000 |
| SEQ ID NO:5334 | 60/223,416 | 08/04/2000 |
| SEQ ID NO:5335 | 60/223,416 | 08/04/2000 |
| SEQ ID NO:5336 | 60/223,416 | 08/04/2000 |
| SEQ ID NO:5337 | 60/223,416 | 08/04/2000 |
| SEQ ID NO:5338 | 60/223,416 | 08/04/2000 |
| SEQ ID NO:5339 | 60/223,416 | 08/04/2000 |
| SEQ ID NO:5340 | 60/223,416 | 08/04/2000 |
| SEQ ID NO:5341 | 60/223,416 | 08/04/2000 |
| SEQ ID NO:5342 | 60/223,416 | 08/04/2000 |
| SEQ ID NO:5343 | 60/223,416 | 08/04/2000 |
| SEQ ID NO:5344 | 60/223,416 | 08/04/2000 |
| SEQ ID NO:5345 | 60/223,416 | 08/04/2000 |
| SEQ ID NO:5346 | 60/223,416 | 08/04/2000 |
| SEQ ID NO:5347 | 60/223,416 | 08/04/2000 |
| SEQ ID NO:5348 | 60/223,416 | 08/04/2000 |
| SEQ ID NO:5349 | 60/223,416 | 08/04/2000 |
| SEQ ID NO:5350 | 60/223,416 | 08/04/2000 |
| SEQ ID NO:5351 | 60/223,416 | 08/04/2000 |
| SEQ ID NO:5352 | 60/223,416 | 08/04/2000 |
| SEQ ID NO:5353 | 60/223,416 | 08/04/2000 |
| SEQ ID NO:5354 | 60/223,416 | 08/04/2000 |
| SEQ ID NO:5355 | 60/223,416 | 08/04/2000 |
| SEQ ID NO:5356 | 60/223,416 | 08/04/2000 |
| SEQ ID NO:5357 | 60/223,416 | 08/04/2000 |
| SEQ ID NO:5358 | 60/223,416 | 08/04/2000 |
| SEQ ID NO:5359 | 60/223,416 | 08/04/2000 |
| SEQ ID NO:5360 | 60/223,416 | 08/04/2000 |
| SEQ ID NO:5361 | 60/223,416 | 08/04/2000 |
| SEQ ID NO:5362 | 60/223,416 | 08/04/2000 |
| SEQ ID NO:5363 | 60/223,416 | 08/04/2000 |
| SEQ ID NO:5364 | 60/223,416 | 08/04/2000 |
| SEQ ID NO:5365 | 60/223,416 | 08/04/2000 |
| SEQ ID NO:5366 | 60/223,416 | 08/04/2000 |
| SEQ ID NO:5367 | 60/223,416 | 08/04/2000 |
| SEQ ID NO:5368 | 60/223,416 | 08/04/2000 |
| SEQ ID NO:5369 | 60/223,416 | 08/04/2000 |
| SEQ ID NO:5370 | 60/223,416 | 08/04/2000 |
| SEQ ID NO:5371 | 60/223,416 | 08/04/2000 |
| SEQ ID NO:5372 | 60/223,416 | 08/04/2000 |
| SEQ ID NO:5373 | 60/223,416 | 08/04/2000 |
| SEQ ID NO:5374 | 60/223,416 | 08/04/2000 |
| SEQ ID NO:5375 | 60/223,416 | 08/04/2000 |
| SEQ ID NO:5376 | 60/223,416 | 08/04/2000 |
| SEQ ID NO:5377 | 60/223,416 | 08/04/2000 |
| SEQ ID NO:5378 | 60/223,416 | 08/04/2000 |
| SEQ ID NO:5379 | 60/223,416 | 08/04/2000 |
| SEQ ID NO:5380 | 60/223,416 | 08/04/2000 |
| SEQ ID NO:5381 | 60/223,416 | 08/04/2000 |
| SEQ ID NO:5382 | 60/223,416 | 08/04/2000 |
| SEQ ID NO:5383 | 60/223,416 | 08/04/2000 |
| SEQ ID NO:5384 | 60/223,416 | 08/04/2000 |
| SEQ ID NO:5385 | 60/223,416 | 08/04/2000 |
| SEQ ID NO:5386 | 60/223,416 | 08/04/2000 |
| SEQ ID NO:5387 | 60/223,416 | 08/04/2000 |
| SEQ ID NO:5388 | 60/223,416 | 08/04/2000 |
| SEQ ID NO:5389 | 60/223,416 | 08/04/2000 |
| SEQ ID NO:5390 | 60/223,416 | 08/04/2000 |
| SEQ ID NO:5391 | 60/223,416 | 08/04/2000 |
| SEQ ID NO:5392 | 60/223,416 | 08/04/2000 |
| SEQ ID NO:5393 | 60/223,416 | 08/04/2000 |
| SEQ ID NO:5394 | 60/223,416 | 08/04/2000 |
| SEQ ID NO:5395 | 60/223,416 | 08/04/2000 |
| SEQ ID NO:5396 | 60/223,416 | 08/04/2000 |
| SEQ ID NO:5397 | 60/223,416 | 08/04/2000 |
| SEQ ID NO:5398 | 60/223,416 | 08/04/2000 |
| SEQ ID NO:5399 | 60/223,416 | 08/04/2000 |
| SEQ ID NO:5400 | 60/223,416 | 08/04/2000 |
| SEQ ID NO:5401 | 60/223,416 | 08/04/2000 |
| SEQ ID NO:5402 | 60/223,416 | 08/04/2000 |
| SEQ ID NO:5403 | 60/223,416 | 08/04/2000 |
| SEQ ID NO:5404 | 60/223,416 | 08/04/2000 |
| SEQ ID NO:5405 | 60/223,416 | 08/04/2000 |
| SEQ ID NO:5406 | 60/223,416 | 08/04/2000 |
| SEQ ID NO:5407 | 60/223,416 | 08/04/2000 |
| SEQ ID NO:5408 | 60/223,416 | 08/04/2000 |
| SEQ ID NO:5409 | 60/223,416 | 08/04/2000 |
| SEQ ID NO:5410 | 60/223,416 | 08/04/2000 |
| SEQ ID NO:5411 | 60/223,416 | 08/04/2000 |
| SEQ ID NO:5412 | 60/223,416 | 08/04/2000 |
| SEQ ID NO:5413 | 60/223,416 | 08/04/2000 |
| SEQ ID NO:5414 | 60/223,416 | 08/04/2000 |
| SEQ ID NO:5415 | 60/223,416 | 08/04/2000 |
| SEQ ID NO:5416 | 60/223,416 | 08/04/2000 |
| SEQ ID NO:5417 | 60/223,416 | 08/04/2000 |
| SEQ ID NO:5418 | 60/223,416 | 08/04/2000 |
| SEQ ID NO:5419 | 60/223,416 | 08/04/2000 |
| SEQ ID NO:5420 | 60/223,416 | 08/04/2000 |
| SEQ ID NO:5421 | 60/223,416 | 08/04/2000 |
| SEQ ID NO:5422 | 60/223,416 | 08/04/2000 |
| SEQ ID NO:5423 | 60/223,416 | 08/04/2000 |
| SEQ ID NO:5424 | 60/223,416 | 08/04/2000 |
| SEQ ID NO:5425 | 60/223,416 | 08/04/2000 |
| SEQ ID NO:5426 | 60/223,416 | 08/04/2000 |
| SEQ ID NO:5427 | 60/223,416 | 08/04/2000 |
| SEQ ID NO:5428 | 60/223,416 | 08/04/2000 |
| SEQ ID NO:5429 | 60/223,416 | 08/04/2000 |
| SEQ ID NO:5430 | 60/223,416 | 08/04/2000 |
| SEQ ID NO:5431 | 60/223,416 | 08/04/2000 |
| SEQ ID NO:5432 | 60/223,416 | 08/04/2000 |
| SEQ ID NO:5433 | 60/223,416 | 08/04/2000 |
| SEQ ID NO:5434 | 60/223,416 | 08/04/2000 |
| SEQ ID NO:5435 | 60/223,416 | 08/04/2000 |
| SEQ ID NO:5436 | 60/223,416 | 08/04/2000 |
| SEQ ID NO:5437 | 60/223,416 | 08/04/2000 |
| SEQ ID NO:5438 | 60/223,416 | 08/04/2000 |
| SEQ ID NO:5439 | 60/223,416 | 08/04/2000 |
| SEQ ID NO:5440 | 60/223,416 | 08/04/2000 |
| SEQ ID NO:5441 | 60/223,416 | 08/04/2000 |
| SEQ ID NO:5442 | 60/223,416 | 08/04/2000 |
| SEQ ID NO:5443 | 60/223,416 | 08/04/2000 |
| SEQ ID NO:5444 | 60/223,416 | 08/04/2000 |
| SEQ ID NO:5445 | 60/223,416 | 08/04/2000 |
| SEQ ID NO:5446 | 60/223,416 | 08/04/2000 |
| SEQ ID NO:5447 | 60/223,416 | 08/04/2000 |
| SEQ ID NO:5448 | 60/223,416 | 08/04/2000 |
| SEQ ID NO:5449 | 60/223,416 | 08/04/2000 |
| SEQ ID NO:5450 | 60/223,416 | 08/04/2000 |
| SEQ ID NO:5451 | 60/223,416 | 08/04/2000 |
| SEQ ID NO:5452 | 60/223,416 | 08/04/2000 |
| SEQ ID NO:5453 | 60/223,416 | 08/04/2000 |
| SEQ ID NO:5454 | 60/223,416 | 08/04/2000 |
| SEQ ID NO:5455 | 60/223,416 | 08/04/2000 |
| SEQ ID NO:5456 | 60/223,416 | 08/04/2000 |
| SEQ ID NO:5457 | 60/223,416 | 08/04/2000 |
| SEQ ID NO:5458 | 60/223,416 | 08/04/2000 |
| SEQ ID NO:5459 | 60/223,416 | 08/04/2000 |
| SEQ ID NO:5460 | 60/223,416 | 08/04/2000 |
| SEQ ID NO:5461 | 60/223,416 | 08/04/2000 |
| SEQ ID NO:5462 | 60/223,416 | 08/04/2000 |
| SEQ ID NO:5463 | 60/223,416 | 08/04/2000 |
| SEQ ID NO:5464 | 60/223,416 | 08/04/2000 |
| SEQ ID NO:5465 | 60/223,416 | 08/04/2000 |
| SEQ ID NO:5466 | 60/223,416 | 08/04/2000 |
| SEQ ID NO:5467 | 60/223,416 | 08/04/2000 |
| SEQ ID NO:5468 | 60/223,416 | 08/04/2000 |
| SEQ ID NO:5469 | 60/223,416 | 08/04/2000 |
| SEQ ID NO:5470 | 60/223,416 | 08/04/2000 |
| SEQ ID NO:5471 | 60/223,416 | 08/04/2000 |
| SEQ ID NO:5472 | 60/223,416 | 08/04/2000 |
| SEQ ID NO:5473 | 60/223,416 | 08/04/2000 |
| SEQ ID NO:5474 | 60/223,416 | 08/04/2000 |
| SEQ ID NO:5475 | 60/223,416 | 08/04/2000 |
| SEQ ID NO:5476 | 60/223,416 | 08/04/2000 |
| SEQ ID NO:5477 | 60/223,416 | 08/04/2000 |
| SEQ ID NO:5478 | 60/223,416 | 08/04/2000 |
| SEQ ID NO:5479 | 60/223,416 | 08/04/2000 |
| SEQ ID NO:5480 | 60/223,416 | 08/04/2000 |
| SEQ ID NO:5481 | 60/223,416 | 08/04/2000 |
| SEQ ID NO:5482 | 60/223,416 | 08/04/2000 |
| SEQ ID NO:5483 | 60/223,416 | 08/04/2000 |
| SEQ ID NO:5484 | 60/223,416 | 08/04/2000 |
| SEQ ID NO:5485 | 60/223,416 | 08/04/2000 |
| SEQ ID NO:5486 | 60/223,416 | 08/04/2000 |
| SEQ ID NO:5487 | 60/223,416 | 08/04/2000 |
| SEQ ID NO:5488 | 60/223,416 | 08/04/2000 |
| SEQ ID NO:5489 | 60/223,416 | 08/04/2000 |
| SEQ ID NO:5490 | 60/223,416 | 08/04/2000 |
| SEQ ID NO:5491 | 60/223,416 | 08/04/2000 |
| SEQ ID NO:5492 | 60/223,416 | 08/04/2000 |
| SEQ ID NO:5493 | 60/223,416 | 08/04/2000 |
| SEQ ID NO:5494 | 60/223,416 | 08/04/2000 |
| SEQ ID NO:5495 | 60/223,416 | 08/04/2000 |
| SEQ ID NO:5496 | 60/223,416 | 08/04/2000 |
| SEQ ID NO:5497 | 60/223,416 | 08/04/2000 |
| SEQ ID NO:5498 | 60/223,416 | 08/04/2000 |
| SEQ ID NO:5499 | 60/223,416 | 08/04/2000 |
| SEQ ID NO:5500 | 60/223,416 | 08/04/2000 |
| SEQ ID NO:5501 | 60/223,416 | 08/04/2000 |
| SEQ ID NO:5502 | 60/223,416 | 08/04/2000 |
| SEQ ID NO:5503 | 60/223,416 | 08/04/2000 |
| SEQ ID NO:5504 | 60/223,416 | 08/04/2000 |
| SEQ ID NO:5505 | 60/223,416 | 08/04/2000 |
| SEQ ID NO:5506 | 60/223,416 | 08/04/2000 |
| SEQ ID NO:5507 | 60/223,416 | 08/04/2000 |
| SEQ ID NO:5508 | 60/223,416 | 08/04/2000 |
| SEQ ID NO:5509 | 60/223,416 | 08/04/2000 |
| SEQ ID NO:5510 | 60/223,416 | 08/04/2000 |
| SEQ ID NO:5511 | 60/223,416 | 08/04/2000 |
| SEQ ID NO:5512 | 60/223,416 | 08/04/2000 |
| SEQ ID NO:5513 | 60/223,416 | 08/04/2000 |
| SEQ ID NO:5514 | 60/223,416 | 08/04/2000 |
| SEQ ID NO:5515 | 60/223,416 | 08/04/2000 |
| SEQ ID NO:5516 | 60/223,416 | 08/04/2000 |
| SEQ ID NO:5517 | 60/223,416 | 08/04/2000 |
| SEQ ID NO:5518 | 60/223,416 | 08/04/2000 |
| SEQ ID NO:5519 | 60/223,416 | 08/04/2000 |
| SEQ ID NO:5520 | 60/223,416 | 08/04/2000 |
| SEQ ID NO:5521 | 60/223,416 | 08/04/2000 |
| SEQ ID NO:5522 | 60/223,416 | 08/04/2000 |
| SEQ ID NO:5523 | 60/223,416 | 08/04/2000 |
| SEQ ID NO:5524 | 60/223,416 | 08/04/2000 |
| SEQ ID NO:5525 | 60/223,416 | 08/04/2000 |
| SEQ ID NO:5526 | 60/223,416 | 08/04/2000 |
| SEQ ID NO:5527 | 60/223,416 | 08/04/2000 |
| SEQ ID NO:5528 | 60/223,416 | 08/04/2000 |
| SEQ ID NO:5529 | 60/223,416 | 08/04/2000 |
| SEQ ID NO:5530 | 60/223,416 | 08/04/2000 |
| SEQ ID NO:5531 | 60/223,416 | 08/04/2000 |
| SEQ ID NO:5532 | 60/223,416 | 08/04/2000 |
| SEQ ID NO:5533 | 60/223,416 | 08/04/2000 |
| SEQ ID NO:5534 | 60/223,416 | 08/04/2000 |
| SEQ ID NO:5535 | 60/223,416 | 08/04/2000 |
| SEQ ID NO:5536 | 60/223,416 | 08/04/2000 |
| SEQ ID NO:5537 | 60/223,416 | 08/04/2000 |
| SEQ ID NO:5538 | 60/223,416 | 08/04/2000 |
| SEQ ID NO:5539 | 60/223,416 | 08/04/2000 |
| SEQ ID NO:5540 | 60/223,416 | 08/04/2000 |
| SEQ ID NO:5541 | 60/223,416 | 08/04/2000 |
| SEQ ID NO:5542 | 60/223,416 | 08/04/2000 |
| SEQ ID NO:5543 | 60/223,416 | 08/04/2000 |
| SEQ ID NO:5544 | 60/223,416 | 08/04/2000 |
| SEQ ID NO:5545 | 60/223,416 | 08/04/2000 |
| SEQ ID NO:5546 | 60/223,416 | 08/04/2000 |
| SEQ ID NO:5547 | 60/223,416 | 08/04/2000 |
| SEQ ID NO:5548 | 60/223,416 | 08/04/2000 |
| SEQ ID NO:5549 | 60/223,416 | 08/04/2000 |
| SEQ ID NO:5550 | 60/223,416 | 08/04/2000 |
| SEQ ID NO:5551 | 60/223,416 | 08/04/2000 |
| SEQ ID NO:5552 | 60/223,416 | 08/04/2000 |
| SEQ ID NO:5553 | 60/223,416 | 08/04/2000 |
| SEQ ID NO:5554 | 60/223,416 | 08/04/2000 |
| SEQ ID NO:5555 | 60/223,416 | 08/04/2000 |
| SEQ ID NO:5556 | 60/223,416 | 08/04/2000 |
| SEQ ID NO:5557 | 60/223,416 | 08/04/2000 |
| SEQ ID NO:5558 | 60/223,416 | 08/04/2000 |
| SEQ ID NO:5559 | 60/223,416 | 08/04/2000 |
| SEQ ID NO:5560 | 60/223,416 | 08/04/2000 |
| SEQ ID NO:5561 | 60/223,416 | 08/04/2000 |
| SEQ ID NO:5562 | 60/223,416 | 08/04/2000 |
| SEQ ID NO:5563 | 60/223,416 | 08/04/2000 |
| SEQ ID NO:5564 | 60/223,416 | 08/04/2000 |
| SEQ ID NO:5565 | 60/223,416 | 08/04/2000 |
| SEQ ID NO:5566 | 60/223,416 | 08/04/2000 |
| SEQ ID NO:5567 | 60/223,416 | 08/04/2000 |
| SEQ ID NO:5568 | 60/223,416 | 08/04/2000 |
| SEQ ID NO:5569 | 60/223,416 | 08/04/2000 |
| SEQ ID NO:5570 | 60/223,416 | 08/04/2000 |
| SEQ ID NO:5571 | 60/223,416 | 08/04/2000 |
| SEQ ID NO:5572 | 60/223,416 | 08/04/2000 |
| SEQ ID NO:5573 | 60/223,416 | 08/04/2000 |
| SEQ ID NO:5574 | 60/223,416 | 08/04/2000 |
| SEQ ID NO:5575 | 60/223,416 | 08/04/2000 |
| SEQ ID NO:5576 | 60/223,416 | 08/04/2000 |
| SEQ ID NO:5577 | 60/223,416 | 08/04/2000 |
| SEQ ID NO:5578 | 60/223,416 | 08/04/2000 |
| SEQ ID NO:5579 | 60/223,416 | 08/04/2000 |
| SEQ ID NO:5580 | 60/223,416 | 08/04/2000 |
| SEQ ID NO:5581 | 60/223,416 | 08/04/2000 |
| SEQ ID NO:5582 | 60/223,416 | 08/04/2000 |
| SEQ ID NO:5583 | 60/223,416 | 08/04/2000 |
| SEQ ID NO:5584 | 60/223,416 | 08/04/2000 |
| SEQ ID NO:5585 | 60/223,416 | 08/04/2000 |
| SEQ ID NO:5586 | 60/223,416 | 08/04/2000 |
| SEQ ID NO:5587 | 60/223,416 | 08/04/2000 |
| SEQ ID NO:5588 | 60/223,416 | 08/04/2000 |
| SEQ ID NO:5589 | 60/223,416 | 08/04/2000 |
| SEQ ID NO:5590 | 60/223,416 | 08/04/2000 |
| SEQ ID NO:5591 | 60/223,416 | 08/04/2000 |
| SEQ ID NO:5592 | 60/223,416 | 08/04/2000 |
| SEQ ID NO:5593 | 60/223,416 | 08/04/2000 |
| SEQ ID NO:5594 | 60/223,416 | 08/04/2000 |
| SEQ ID NO:5595 | 60/223,416 | 08/04/2000 |
| SEQ ID NO:5596 | 60/223,416 | 08/04/2000 |
| SEQ ID NO:5597 | 60/223,416 | 08/04/2000 |
| SEQ ID NO:5598 | 60/223,416 | 08/04/2000 |
| SEQ ID NO:5599 | 60/223,416 | 08/04/2000 |
| SEQ ID NO:5600 | 60/223,416 | 08/04/2000 |
| SEQ ID NO:5601 | 60/223,416 | 08/04/2000 |
| SEQ ID NO:5602 | 60/223,416 | 08/04/2000 |
| SEQ ID NO:5603 | 60/223,416 | 08/04/2000 |
| SEQ ID NO:5604 | 60/223,416 | 08/04/2000 |
| SEQ ID NO:5605 | 60/223,416 | 08/04/2000 |
| SEQ ID NO:5606 | 60/223,416 | 08/04/2000 |
| SEQ ID NO:5607 | 60/223,416 | 08/04/2000 |
| SEQ ID NO:5608 | 60/223,416 | 08/04/2000 |
| SEQ ID NO:5609 | 60/223,416 | 08/04/2000 |
| SEQ ID NO:5610 | 60/223,416 | 08/04/2000 |
| SEQ ID NO:5611 | 60/223,416 | 08/04/2000 |
| SEQ ID NO:5612 | 60/223,416 | 08/04/2000 |
| SEQ ID NO:5613 | 60/223,416 | 08/04/2000 |
| SEQ ID NO:5614 | 60/223,416 | 08/04/2000 |
| SEQ ID NO:5615 | 60/223,416 | 08/04/2000 |
| SEQ ID NO:5616 | 60/223,416 | 08/04/2000 |
| SEQ ID NO:5617 | 60/223,416 | 08/04/2000 |
| SEQ ID NO:5618 | 60/223,416 | 08/04/2000 |
| SEQ ID NO:5619 | 60/223,416 | 08/04/2000 |
| SEQ ID NO:5620 | 60/223,416 | 08/04/2000 |
| SEQ ID NO:5621 | 60/223,416 | 08/04/2000 |
| SEQ ID NO:5622 | 60/223,416 | 08/04/2000 |
| SEQ ID NO:5623 | 60/223,416 | 08/04/2000 |
| SEQ ID NO:5624 | 60/223,416 | 08/04/2000 |
| SEQ ID NO:5625 | 60/223,416 | 08/04/2000 |
| SEQ ID NO:5626 | 60/223,416 | 08/04/2000 |
| SEQ ID NO:5627 | 60/223,416 | 08/04/2000 |
| SEQ ID NO:5628 | 60/223,416 | 08/04/2000 |
| SEQ ID NO:5629 | 60/223,416 | 08/04/2000 |
| SEQ ID NO:5630 | 60/223,416 | 08/04/2000 |
| SEQ ID NO:5631 | 60/223,416 | 08/04/2000 |
| SEQ ID NO:5632 | 60/223,416 | 08/04/2000 |
| SEQ ID NO:5633 | 60/223,416 | 08/04/2000 |
| SEQ ID NO:5634 | 60/223,416 | 08/04/2000 |
| SEQ ID NO:5635 | 60/223,416 | 08/04/2000 |
| SEQ ID NO:5636 | 60/223,416 | 08/04/2000 |
| SEQ ID NO:5637 | 60/223,416 | 08/04/2000 |
| SEQ ID NO:5638 | 60/223,416 | 08/04/2000 |
| SEQ ID NO:5639 | 60/223,416 | 08/04/2000 |
| SEQ ID NO:5640 | 60/223,416 | 08/04/2000 |
| SEQ ID NO:5641 | 60/223,416 | 08/04/2000 |
| SEQ ID NO:5642 | 60/223,416 | 08/04/2000 |
| SEQ ID NO:5643 | 60/223,416 | 08/04/2000 |
| SEQ ID NO:5644 | 60/223,416 | 08/04/2000 |
| SEQ ID NO:5645 | 60/223,416 | 08/04/2000 |
| SEQ ID NO:5646 | 60/223,416 | 08/04/2000 |
| SEQ ID NO:5647 | 60/223,416 | 08/04/2000 |
| SEQ ID NO:5648 | 60/223,416 | 08/04/2000 |
| SEQ ID NO:5649 | 60/223,416 | 08/04/2000 |
| SEQ ID NO:5650 | 60/223,416 | 08/04/2000 |
| SEQ ID NO:5651 | 60/223,416 | 08/04/2000 |
| SEQ ID NO:5652 | 60/223,416 | 08/04/2000 |
| SEQ ID NO:5653 | 60/223,416 | 08/04/2000 |
| SEQ ID NO:5654 | 60/223,416 | 08/04/2000 |
| SEQ ID NO:5655 | 60/223,416 | 08/04/2000 |
| SEQ ID NO:5656 | 60/223,416 | 08/04/2000 |
| SEQ ID NO:5657 | 60/223,416 | 08/04/2000 |
| SEQ ID NO:5658 | 60/223,416 | 08/04/2000 |
| SEQ ID NO:5659 | 60/223,416 | 08/04/2000 |
| SEQ ID NO:5660 | 60/223,416 | 08/04/2000 |
| SEQ ID NO:5661 | 60/223,416 | 08/04/2000 |
| SEQ ID NO:5662 | 60/223,416 | 08/04/2000 |
| SEQ ID NO:5663 | 60/223,416 | 08/04/2000 |
| SEQ ID NO:5664 | 60/223,416 | 08/04/2000 |
| SEQ ID NO:5665 | 60/223,416 | 08/04/2000 |
| SEQ ID NO:5666 | 60/223,416 | 08/04/2000 |
| SEQ ID NO:5667 | 60/223,416 | 08/04/2000 |
| SEQ ID NO:5668 | 60/223,416 | 08/04/2000 |
| SEQ ID NO:5669 | 60/223,416 | 08/04/2000 |
| SEQ ID NO:5670 | 60/223,416 | 08/04/2000 |
| SEQ ID NO:5671 | 60/223,416 | 08/04/2000 |
| SEQ ID NO:5672 | 60/223,416 | 08/04/2000 |
| SEQ ID NO:5673 | 60/223,416 | 08/04/2000 |
| SEQ ID NO:5674 | 60/223,416 | 08/04/2000 |
| SEQ ID NO:5675 | 60/223,416 | 08/04/2000 |
| SEQ ID NO:5676 | 60/223,416 | 08/04/2000 |
| SEQ ID NO:5677 | 60/223,416 | 08/04/2000 |
| SEQ ID NO:5678 | 60/223,416 | 08/04/2000 |
| SEQ ID NO:5679 | 60/223,416 | 08/04/2000 |
| SEQ ID NO:5680 | 60/223,416 | 08/04/2000 |
| SEQ ID NO:5681 | 60/223,416 | 08/04/2000 |
| SEQ ID NO:5682 | 60/223,416 | 08/04/2000 |
| SEQ ID NO:5683 | 60/223,416 | 08/04/2000 |
| SEQ ID NO:5684 | 60/223,416 | 08/04/2000 |
| SEQ ID NO:5685 | 60/223,416 | 08/04/2000 |
| SEQ ID NO:5686 | 60/223,416 | 08/04/2000 |
| SEQ ID NO:5687 | 60/223,416 | 08/04/2000 |
| SEQ ID NO:5688 | 60/223,416 | 08/04/2000 |
| SEQ ID NO:5689 | 60/223,416 | 08/04/2000 |
| SEQ ID NO:5690 | 60/223,416 | 08/04/2000 |
| SEQ ID NO:5691 | 60/223,416 | 08/04/2000 |
| SEQ ID NO:5692 | 60/223,416 | 08/04/2000 |
| SEQ ID NO:5693 | 60/223,416 | 08/04/2000 |
| SEQ ID NO:5694 | 60/223,416 | 08/04/2000 |
| SEQ ID NO:5695 | 60/223,416 | 08/04/2000 |
| SEQ ID NO:5696 | 60/223,416 | 08/04/2000 |
| SEQ ID NO:5697 | 60/223,416 | 08/04/2000 |
| SEQ ID NO:5698 | 60/223,416 | 08/04/2000 |
| SEQ ID NO:5699 | 60/223,416 | 08/04/2000 |
| SEQ ID NO:5700 | 60/223,416 | 08/04/2000 |
| SEQ ID NO:5701 | 60/223,416 | 08/04/2000 |
| SEQ ID NO:5702 | 60/223,416 | 08/04/2000 |
| SEQ ID NO:5703 | 60/223,416 | 08/04/2000 |
| SEQ ID NO:5704 | 60/223,416 | 08/04/2000 |
| SEQ ID NO:5705 | 60/223,416 | 08/04/2000 |
| SEQ ID NO:5706 | 60/223,416 | 08/04/2000 |
| SEQ ID NO:5707 | 60/223,416 | 08/04/2000 |
| SEQ ID NO:5708 | 60/223,416 | 08/04/2000 |
| SEQ ID NO:5709 | 60/223,416 | 08/04/2000 |
| SEQ ID NO:5710 | 60/223,416 | 08/04/2000 |
| SEQ ID NO:5711 | 60/223,416 | 08/04/2000 |
| SEQ ID NO:5712 | 60/223,416 | 08/04/2000 |
| SEQ ID NO:5713 | 60/223,416 | 08/04/2000 |
| SEQ ID NO:5714 | 60/223,416 | 08/04/2000 |
| SEQ ID NO:5715 | 60/223,416 | 08/04/2000 |
| SEQ ID NO:5716 | 60/223,416 | 08/04/2000 |
| SEQ ID NO:5717 | 60/223,416 | 08/04/2000 |
| SEQ ID NO:5718 | 60/223,416 | 08/04/2000 |
| SEQ ID NO:5719 | 60/223,416 | 08/04/2000 |
| SEQ ID NO:5720 | 60/223,416 | 08/04/2000 |
| SEQ ID NO:5721 | 60/223,416 | 08/04/2000 |
| SEQ ID NO:5722 | 60/223,416 | 08/04/2000 |
| SEQ ID NO:5723 | 60/223,416 | 08/04/2000 |
| SEQ ID NO:5724 | 60/223,416 | 08/04/2000 |
| SEQ ID NO:5725 | 60/223,416 | 08/04/2000 |
| SEQ ID NO:5726 | 60/223,416 | 08/04/2000 |
| SEQ ID NO:5727 | 60/223,416 | 08/04/2000 |
| SEQ ID NO:5728 | 60/223,416 | 08/04/2000 |
| SEQ ID NO:5729 | 60/223,416 | 08/04/2000 |
| SEQ ID NO:5730 | 60/223,416 | 08/04/2000 |
| SEQ ID NO:5731 | 60/223,416 | 08/04/2000 |
| SEQ ID NO:5732 | 60/223,416 | 08/04/2000 |
| SEQ ID NO:5733 | 60/223,416 | 08/04/2000 |
| SEQ ID NO:5734 | 60/223,416 | 08/04/2000 |
| SEQ ID NO:5735 | 60/223,416 | 08/04/2000 |
| SEQ ID NO:5736 | 60/223,416 | 08/04/2000 |
| SEQ ID NO:5737 | 60/223,416 | 08/04/2000 |
| SEQ ID NO:5738 | 60/223,416 | 08/04/2000 |
| SEQ ID NO:5739 | 60/223,416 | 08/04/2000 |
| SEQ ID NO:5740 | 60/223,416 | 08/04/2000 |
| SEQ ID NO:5741 | 60/223,416 | 08/04/2000 |
| SEQ ID NO:5742 | 60/223,416 | 08/04/2000 |
| SEQ ID NO:5743 | 60/223,416 | 08/04/2000 |
| SEQ ID NO:5744 | 60/223,416 | 08/04/2000 |
| SEQ ID NO:5745 | 60/223,416 | 08/04/2000 |
| SEQ ID NO:5746 | 60/223,416 | 08/04/2000 |
| SEQ ID NO:5747 | 60/223,416 | 08/04/2000 |
| SEQ ID NO:5748 | 60/223,416 | 08/04/2000 |
| SEQ ID NO:5749 | 60/223,416 | 08/04/2000 |
| SEQ ID NO:5750 | 60/223,416 | 08/04/2000 |
| SEQ ID NO:5751 | 60/223,416 | 08/04/2000 |
| SEQ ID NO:5752 | 60/223,416 | 08/04/2000 |
| SEQ ID NO:5753 | 60/223,416 | 08/04/2000 |
| SEQ ID NO:5754 | 60/223,416 | 08/04/2000 |
| SEQ ID NO:5755 | 60/223,416 | 08/04/2000 |
| SEQ ID NO:5756 | 60/223,416 | 08/04/2000 |
| SEQ ID NO:5757 | 60/223,416 | 08/04/2000 |
| SEQ ID NO:5758 | 60/223,416 | 08/04/2000 |
| SEQ ID NO:5759 | 60/223,416 | 08/04/2000 |
| SEQ ID NO:5760 | 60/223,416 | 08/04/2000 |
| SEQ ID NO:5761 | 60/223,416 | 08/04/2000 |
| SEQ ID NO:5762 | 60/223,416 | 08/04/2000 |
| SEQ ID NO:5763 | 60/223,416 | 08/04/2000 |
| SEQ ID NO:5764 | 60/223,416 | 08/04/2000 |
| SEQ ID NO:5765 | 60/223,416 | 08/04/2000 |
| SEQ ID NO:5766 | 60/223,416 | 08/04/2000 |
| SEQ ID NO:5767 | 60/223,416 | 08/04/2000 |
| SEQ ID NO:5768 | 60/223,416 | 08/04/2000 |
| SEQ ID NO:5769 | 60/223,416 | 08/04/2000 |
| SEQ ID NO:5770 | 60/223,416 | 08/04/2000 |
| SEQ ID NO:5771 | 60/223,416 | 08/04/2000 |
| SEQ ID NO:5772 | 60/223,416 | 08/04/2000 |
| SEQ ID NO:5773 | 60/223,416 | 08/04/2000 |
| SEQ ID NO:5774 | 60/223,416 | 08/04/2000 |
| SEQ ID NO:5775 | 60/223,416 | 08/04/2000 |
| SEQ ID NO:5776 | 60/223,416 | 08/04/2000 |
| SEQ ID NO:5777 | 60/223,416 | 08/04/2000 |
| SEQ ID NO:5778 | 60/223,416 | 08/04/2000 |
| SEQ ID NO:5779 | 60/223,416 | 08/04/2000 |
| SEQ ID NO:5780 | 60/223,416 | 08/04/2000 |
| SEQ ID NO:5781 | 60/223,416 | 08/04/2000 |
| SEQ ID NO:5782 | 60/223,416 | 08/04/2000 |
| SEQ ID NO:5783 | 60/223,416 | 08/04/2000 |
| SEQ ID NO:5784 | 60/223,416 | 08/04/2000 |
| SEQ ID NO:5785 | 60/223,416 | 08/04/2000 |
| SEQ ID NO:5786 | 60/223,416 | 08/04/2000 |
| SEQ ID NO:5787 | 60/223,416 | 08/04/2000 |
| SEQ ID NO:5788 | 60/223,416 | 08/04/2000 |
| SEQ ID NO:5789 | 60/223,416 | 08/04/2000 |
| SEQ ID NO:5790 | 60/200,303 | 04/28/2000 |
| SEQ ID NO:5791 | 60/200,303 | 04/28/2000 |
| SEQ ID NO:5792 | 60/200,303 | 04/28/2000 |
| SEQ ID NO:5793 | 60/200,303 | 04/28/2000 |
| SEQ ID NO:5794 | 60/200,303 | 04/28/2000 |
| SEQ ID NO:5795 | 60/200,303 | 04/28/2000 |
| SEQ ID NO:5796 | 60/200,303 | 04/28/2000 |
| SEQ ID NO:5797 | 60/200,303 | 04/28/2000 |
| SEQ ID NO:5798 | 60/200,303 | 04/28/2000 |
| SEQ ID NO:5799 | 60/200,303 | 04/28/2000 |
| SEQ ID NO:5800 | 60/200,303 | 04/28/2000 |
| SEQ ID NO:5801 | 60/200,303 | 04/28/2000 |
| SEQ ID NO:5802 | 60/200,303 | 04/28/2000 |
| SEQ ID NO:5803 | 60/200,303 | 04/28/2000 |
| SEQ ID NO:5804 | 60/200,303 | 04/28/2000 |
| SEQ ID NO:5805 | 60/200,303 | 04/28/2000 |
| SEQ ID NO:5806 | 60/200,303 | 04/28/2000 |
| SEQ ID NO:5807 | 60/200,303 | 04/28/2000 |
| SEQ ID NO:5808 | 60/200,303 | 04/28/2000 |
| SEQ ID NO:5809 | 60/200,303 | 04/28/2000 |
| SEQ ID NO:5810 | 60/200,303 | 04/28/2000 |
| SEQ ID NO:5811 | 60/200,303 | 04/28/2000 |
| SEQ ID NO:5812 | 60/200,303 | 04/28/2000 |
| SEQ ID NO:5813 | 60/200,303 | 04/28/2000 |
| SEQ ID NO:5814 | 60/200,303 | 04/28/2000 |
| SEQ ID NO:5815 | 60/200,303 | 04/28/2000 |
| SEQ ID NO:5816 | 60/200,303 | 04/28/2000 |
| SEQ ID NO:5817 | 60/200,303 | 04/28/2000 |
| SEQ ID NO:5818 | 60/200,303 | 04/28/2000 |
| SEQ ID NO:5819 | 60/200,303 | 04/28/2000 |
| SEQ ID NO:5820 | 60/200,303 | 04/28/2000 |
| SEQ ID NO:5821 | 60/200,303 | 04/28/2000 |
| SEQ ID NO:5822 | 60/200,303 | 04/28/2000 |
| SEQ ID NO:5823 | 60/200,303 | 04/28/2000 |
| SEQ ID NO:5824 | 60/200,303 | 04/28/2000 |
| SEQ ID NO:5825 | 60/200,303 | 04/28/2000 |
| SEQ ID NO:5826 | 60/200,303 | 04/28/2000 |
| SEQ ID NO:5827 | 60/200,303 | 04/28/2000 |
| SEQ ID NO:5828 | 60/200,303 | 04/28/2000 |
| SEQ ID NO:5829 | 60/200,303 | 04/28/2000 |
| SEQ ID NO:5830 | 60/200,303 | 04/28/2000 |
| SEQ ID NO:5831 | 60/200,303 | 04/28/2000 |
| SEQ ID NO:5832 | 60/200,303 | 04/28/2000 |
| SEQ ID NO:5833 | 60/200,303 | 04/28/2000 |
| SEQ ID NO:5834 | 60/200,303 | 04/28/2000 |
| SEQ ID NO:5835 | 60/200,303 | 04/28/2000 |
| SEQ ID NO:5836 | 60/200,303 | 04/28/2000 |
| SEQ ID NO:5837 | 60/200,303 | 04/28/2000 |
| SEQ ID NO:5838 | 60/200,303 | 04/28/2000 |
| SEQ ID NO:5839 | 60/200,303 | 04/28/2000 |
| SEQ ID NO:5840 | 60/200,303 | 04/28/2000 |
| SEQ ID NO:5841 | 60/200,303 | 04/28/2000 |
| SEQ ID NO:5842 | 60/200,303 | 04/28/2000 |
| SEQ ID NO:5843 | 60/200,303 | 04/28/2000 |
| SEQ ID NO:5844 | 60/200,303 | 04/28/2000 |
| SEQ ID NO:5845 | 60/200,303 | 04/28/2000 |
| SEQ ID NO:5846 | 60/200,303 | 04/28/2000 |
| SEQ ID NO:5847 | 60/200,303 | 04/28/2000 |
| SEQ ID NO:5848 | 60/200,303 | 04/28/2000 |
| SEQ ID NO:5849 | 60/200,303 | 04/28/2000 |
| SEQ ID NO:5850 | 60/200,303 | 04/28/2000 |
| SEQ ID NO:5851 | 60/200,303 | 04/28/2000 |
| SEQ ID NO:5852 | 60/200,303 | 04/28/2000 |
| SEQ ID NO:5853 | 60/200,303 | 04/28/2000 |
| SEQ ID NO:5854 | 60/200,303 | 04/28/2000 |
| SEQ ID NO:5855 | 60/200,303 | 04/28/2000 |
| SEQ ID NO:5856 | 60/200,303 | 04/28/2000 |
| SEQ ID NO:5857 | 60/200,303 | 04/28/2000 |
| SEQ ID NO:5858 | 60/200,303 | 04/28/2000 |
| SEQ ID NO:5859 | 60/200,303 | 04/28/2000 |
| SEQ ID NO:5860 | 60/200,303 | 04/28/2000 |
| SEQ ID NO:5861 | 60/200,303 | 04/28/2000 |
| SEQ ID NO:5862 | 60/200,303 | 04/28/2000 |
| SEQ ID NO:5863 | 60/200,303 | 04/28/2000 |
| SEQ ID NO:5864 | 60/200,303 | 04/28/2000 |
| SEQ ID NO:5865 | 60/200,303 | 04/28/2000 |
| SEQ ID NO:5866 | 60/200,303 | 04/28/2000 |
| SEQ ID NO:5867 | 60/200,303 | 04/28/2000 |
| SEQ ID NO:5868 | 60/200,303 | 04/28/2000 |
| SEQ ID NO:5869 | 60/200,303 | 04/28/2000 |
| SEQ ID NO:5870 | 60/200,303 | 04/28/2000 |
| SEQ ID NO:5871 | 60/200,303 | 04/28/2000 |
| SEQ ID NO:5872 | 60/200,303 | 04/28/2000 |
| SEQ ID NO:5873 | 60/200,303 | 04/28/2000 |
| SEQ ID NO:5874 | 60/200,303 | 04/28/2000 |
| SEQ ID NO:5875 | 60/200,303 | 04/28/2000 |
| SEQ ID NO:5876 | 60/200,779 | 05/22/2000 |
| SEQ ID NO:5877 | 60/200,779 | 05/22/2000 |
| SEQ ID NO:5878 | 60/200,779 | 05/22/2000 |
| SEQ ID NO:5879 | 60/200,779 | 05/22/2000 |
| SEQ ID NO:5880 | 60/200,779 | 05/22/2000 |
| SEQ ID NO:5881 | 60/200,779 | 05/22/2000 |
| SEQ ID NO:5882 | 60/200,779 | 05/22/2000 |
| SEQ ID NO:5883 | 60/200,779 | 05/22/2000 |
| SEQ ID NO:5884 | 60/200,779 | 05/22/2000 |
| SEQ ID NO:5885 | 60/200,779 | 05/22/2000 |
| SEQ ID NO:5886 | 60/200,779 | 05/22/2000 |
| SEQ ID NO:5887 | 60/200,779 | 05/22/2000 |
| SEQ ID NO:5888 | 60/200,779 | 05/22/2000 |
| SEQ ID NO:5889 | 60/200,779 | 05/22/2000 |
| SEQ ID NO:5890 | 60/200,779 | 05/22/2000 |
| SEQ ID NO:5891 | 60/200,779 | 05/22/2000 |
| SEQ ID NO:5892 | 60/200,779 | 05/22/2000 |
| SEQ ID NO:5893 | 60/200,779 | 05/22/2000 |
| SEQ ID NO:5894 | 60/200,779 | 05/22/2000 |
| SEQ ID NO:5895 | 60/200,779 | 05/22/2000 |
| SEQ ID NO:5896 | 60/200,779 | 05/22/2000 |
| SEQ ID NO:5897 | 60/200,779 | 05/22/2000 |
| SEQ ID NO:5898 | 60/200,779 | 05/22/2000 |
| SEQ ID NO:5899 | 60/200,779 | 05/22/2000 |
| SEQ ID NO:5900 | 60/200,779 | 05/22/2000 |
| SEQ ID NO:5901 | 60/200,779 | 05/22/2000 |
| SEQ ID NO:5902 | 60/200,779 | 05/22/2000 |
| SEQ ID NO:5903 | 60/200,779 | 05/22/2000 |
| SEQ ID NO:5904 | 60/200,779 | 05/22/2000 |
| SEQ ID NO:5905 | 60/200,779 | 05/22/2000 |
| SEQ ID NO:5906 | 60/200,779 | 05/22/2000 |
| SEQ ID NO:5907 | 60/200,779 | 05/22/2000 |
| SEQ ID NO:5908 | 60/200,779 | 05/22/2000 |
| SEQ ID NO:5909 | 60/200,779 | 05/22/2000 |
| SEQ ID NO:5910 | 60/200,779 | 05/22/2000 |
| SEQ ID NO:5911 | 60/200,779 | 05/22/2000 |
| SEQ ID NO:5912 | 60/200,779 | 05/22/2000 |
| SEQ ID NO:5913 | 60/200,779 | 05/22/2000 |
| SEQ ID NO:5914 | 60/200,779 | 05/22/2000 |
| SEQ ID NO:5915 | 60/200,779 | 05/22/2000 |
| SEQ ID NO:5916 | 60/200,779 | 05/22/2000 |
| SEQ ID NO:5917 | 60/200,779 | 05/22/2000 |
| SEQ ID NO:5918 | 60/200,779 | 05/22/2000 |
| SEQ ID NO:5919 | 60/200,779 | 05/22/2000 |
| SEQ ID NO:5920 | 60/200,779 | 05/22/2000 |
| SEQ ID NO:5921 | 60/200,779 | 05/22/2000 |
| SEQ ID NO:5922 | 60/200,779 | 05/22/2000 |
| SEQ ID NO:5923 | 60/200,779 | 05/22/2000 |
| SEQ ID NO:5924 | 60/200,779 | 05/22/2000 |
| SEQ ID NO:5925 | 60/200,779 | 05/22/2000 |
| SEQ ID NO:5926 | 60/200,779 | 05/22/2000 |
| SEQ ID NO:5927 | 60/200,779 | 05/22/2000 |
| SEQ ID NO:5928 | 60/200,779 | 05/22/2000 |
| SEQ ID NO:5929 | 60/200,779 | 05/22/2000 |
| SEQ ID NO:5930 | 60/200,779 | 05/22/2000 |
| SEQ ID NO:5931 | 60/200,779 | 05/22/2000 |
| SEQ ID NO:5932 | 60/200,779 | 05/22/2000 |
| SEQ ID NO:5933 | 60/200,779 | 05/22/2000 |
| SEQ ID NO:5934 | 60/200,779 | 05/22/2000 |
| SEQ ID NO:5935 | 60/200,779 | 05/22/2000 |
| SEQ ID NO:5936 | 60/200,779 | 05/22/2000 |
| SEQ ID NO:5937 | 60/200,779 | 05/22/2000 |
| SEQ ID NO:5938 | 60/200,779 | 05/22/2000 |
| SEQ ID NO:5939 | 60/200,779 | 05/22/2000 |
| SEQ ID NO:5940 | 60/200,779 | 05/22/2000 |
| SEQ ID NO:5941 | 60/200,779 | 05/22/2000 |
| SEQ ID NO:5942 | 60/200,779 | 05/22/2000 |
| SEQ ID NO:5943 | 60/200,779 | 05/22/2000 |
| SEQ ID NO:5944 | 60/200,779 | 05/22/2000 |
| SEQ ID NO:5945 | 60/200,779 | 05/22/2000 |
| SEQ ID NO:5946 | 60/200,779 | 05/22/2000 |
| SEQ ID NO:5947 | 60/200,779 | 05/22/2000 |
| SEQ ID NO:5948 | 60/200,779 | 05/22/2000 |
| SEQ ID NO:5949 | 60/200,779 | 05/22/2000 |
| SEQ ID NO:5950 | 60/200,779 | 05/22/2000 |
| SEQ ID NO:5951 | 60/200,779 | 05/22/2000 |
| SEQ ID NO:5952 | 60/200,779 | 05/22/2000 |
| SEQ ID NO:5953 | 60/200,779 | 05/22/2000 |
| SEQ ID NO:5954 | 60/200,779 | 05/22/2000 |
| SEQ ID NO:5955 | 60/200,779 | 05/22/2000 |
| SEQ ID NO:5956 | 60/200,779 | 05/22/2000 |
| SEQ ID NO:5957 | 60/200,779 | 05/22/2000 |
| SEQ ID NO:5958 | 60/200,779 | 05/22/2000 |
| SEQ ID NO:5959 | 60/200,779 | 05/22/2000 |
| SEQ ID NO:5960 | 60/200,779 | 05/22/2000 |
| SEQ ID NO:5961 | 60/200,779 | 05/22/2000 |
| SEQ ID NO:5962 | 60/200,779 | 05/22/2000 |
| SEQ ID NO:5963 | 60/200,779 | 05/22/2000 |
| SEQ ID NO:5964 | 60/200,779 | 05/22/2000 |
| SEQ ID NO:5965 | 60/200,779 | 05/22/2000 |
| SEQ ID NO:5966 | 60/200,779 | 05/22/2000 |
| SEQ ID NO:5967 | 60/200,779 | 05/22/2000 |
| SEQ ID NO:5968 | 60/200,779 | 05/22/2000 |
| SEQ ID NO:5969 | 60/200,779 | 05/22/2000 |
| SEQ ID NO:5970 | 60/200,779 | 05/22/2000 |
| SEQ ID NO:5971 | 60/200,779 | 05/22/2000 |
| SEQ ID NO:5972 | 60/200,779 | 05/22/2000 |
| SEQ ID NO:5973 | 60/200,779 | 05/22/2000 |
| SEQ ID NO:5974 | 60/200,779 | 05/22/2000 |
| SEQ ID NO:5975 | 60/200,779 | 05/22/2000 |
| SEQ ID NO:5976 | 60/200,779 | 05/22/2000 |
| SEQ ID NO:5977 | 60/200,779 | 05/22/2000 |
| SEQ ID NO:5978 | 60/200,779 | 05/22/2000 |
| SEQ ID NO:5979 | 60/200,779 | 05/22/2000 |
| SEQ ID NO:5980 | 60/200,779 | 05/22/2000 |
| SEQ ID NO:5981 | 60/200,779 | 05/22/2000 |
| SEQ ID NO:5982 | 60/200,779 | 05/22/2000 |
| SEQ ID NO:5983 | 60/200,779 | 05/22/2000 |
| SEQ ID NO:5984 | 60/200,779 | 05/22/2000 |
| SEQ ID NO:5985 | 60/200,779 | 05/22/2000 |
| SEQ ID NO:5986 | 60/200,779 | 05/22/2000 |
| SEQ ID NO:5987 | 60/200,779 | 05/22/2000 |
| SEQ ID NO:5988 | 60/200,779 | 05/22/2000 |
| SEQ ID NO:5989 | 60/200,779 | 05/22/2000 |
| SEQ ID NO:5990 | 60/200,779 | 05/22/2000 |
| SEQ ID NO:5991 | 60/200,779 | 05/22/2000 |
| SEQ ID NO:5992 | 60/200,779 | 05/22/2000 |
| SEQ ID NO:5993 | 60/200,779 | 05/22/2000 |
| SEQ ID NO:5994 | 60/200,779 | 05/22/2000 |
| SEQ ID NO:5995 | 60/200,779 | 05/22/2000 |
| SEQ ID NO:5996 | 60/200,779 | 05/22/2000 |
| SEQ ID NO:5997 | 60/200,779 | 05/22/2000 |
| SEQ ID NO:5998 | 60/200,779 | 05/22/2000 |
| SEQ ID NO:5999 | 60/200,779 | 05/22/2000 |
| SEQ ID NO:6000 | 60/200,779 | 05/22/2000 |
| SEQ ID NO:6001 | 60/200,779 | 05/22/2000 |
| SEQ ID NO:6002 | 60/200,779 | 05/22/2000 |
| SEQ ID NO:6003 | 60/200,779 | 05/22/2000 |
| SEQ ID NO:6004 | 60/200,779 | 05/22/2000 |
| SEQ ID NO:6005 | 60/200,779 | 05/22/2000 |
| SEQ ID NO:6006 | 60/200,779 | 05/22/2000 |
| SEQ ID NO:6007 | 60/200,779 | 05/22/2000 |
| SEQ ID NO:6008 | 60/200,779 | 05/22/2000 |
| SEQ ID NO:6009 | 60/200,779 | 05/22/2000 |
| SEQ ID NO:6010 | 60/200,779 | 05/22/2000 |
| SEQ ID NO:6011 | 60/200,779 | 05/22/2000 |
| SEQ ID NO:6012 | 60/200,779 | 05/22/2000 |
| SEQ ID NO:6013 | 60/200,779 | 05/22/2000 |
| SEQ ID NO:6014 | 60/200,779 | 05/22/2000 |
| SEQ ID NO:6015 | 60/200,779 | 05/22/2000 |
| SEQ ID NO:6016 | 60/200,779 | 05/22/2000 |
| SEQ ID NO:6017 | 60/200,779 | 05/22/2000 |
| SEQ ID NO:6018 | 60/200,779 | 05/22/2000 |
| SEQ ID NO:6019 | 60/200,779 | 05/22/2000 |
| SEQ ID NO:6020 | 60/200,779 | 05/22/2000 |
| SEQ ID NO:6021 | 60/200,779 | 05/22/2000 |
| SEQ ID NO:6022 | 60/200,779 | 05/22/2000 |
| SEQ ID NO:6023 | 60/200,779 | 05/22/2000 |
| SEQ ID NO:6024 | 60/200,779 | 05/22/2000 |
| SEQ ID NO:6025 | 60/200,779 | 05/22/2000 |
| SEQ ID NO:6026 | 60/200,779 | 05/22/2000 |
| SEQ ID NO:6027 | 60/200,779 | 05/22/2000 |
| SEQ ID NO:6028 | 60/200,779 | 05/22/2000 |
| SEQ ID NO:6029 | 60/200,779 | 05/22/2000 |
| SEQ ID NO:6030 | 60/200,779 | 05/22/2000 |
| SEQ ID NO:6031 | 60/200,779 | 05/22/2000 |
| SEQ ID NO:6032 | 60/200,779 | 05/22/2000 |
| SEQ ID NO:6033 | 60/200,779 | 05/22/2000 |
| SEQ ID NO:6034 | 60/200,779 | 05/22/2000 |
| SEQ ID NO:6035 | 60/200,779 | 05/22/2000 |
| SEQ ID NO:6036 | 60/200,779 | 05/22/2000 |
| SEQ ID NO:6037 | 60/200,779 | 05/22/2000 |
| SEQ ID NO:6038 | 60/200,779 | 05/22/2000 |
| SEQ ID NO:6039 | 60/200,779 | 05/22/2000 |
| SEQ ID NO:6040 | 60/200,779 | 05/22/2000 |
| SEQ ID NO:6041 | 60/200,779 | 05/22/2000 |
| SEQ ID NO:6042 | 60/200,779 | 05/22/2000 |
| SEQ ID NO:6043 | 60/200,779 | 05/22/2000 |
| SEQ ID NO:6044 | 60/200,779 | 05/22/2000 |
| SEQ ID NO:6045 | 60/200,779 | 05/22/2000 |
| SEQ ID NO:6046 | 60/200,779 | 05/22/2000 |
| SEQ ID NO:6047 | 60/200,779 | 05/22/2000 |
| SEQ ID NO:6048 | 60/200,779 | 05/22/2000 |
| SEQ ID NO:6049 | 60/200,779 | 05/22/2000 |
| SEQ ID NO:6050 | 60/200,779 | 05/22/2000 |
| SEQ ID NO:6051 | 60/200,779 | 05/22/2000 |
| SEQ ID NO:6052 | 60/200,779 | 05/22/2000 |
| SEQ ID NO:6053 | 60/200,779 | 05/22/2000 |
| SEQ ID NO:6054 | 60/200,779 | 05/22/2000 |
| SEQ ID NO:6055 | 60/200,779 | 05/22/2000 |
| SEQ ID NO:6056 | 60/200,779 | 05/22/2000 |
| SEQ ID NO:6057 | 60/200,779 | 05/22/2000 |
| SEQ ID NO:6058 | 60/200,779 | 05/22/2000 |
| SEQ ID NO:6059 | 60/200,779 | 05/22/2000 |
| SEQ ID NO:6060 | 60/200,779 | 05/22/2000 |
| SEQ ID NO:6061 | 60/200,779 | 05/22/2000 |
| SEQ ID NO:6062 | 60/200,779 | 05/22/2000 |
| SEQ ID NO:6063 | 60/200,779 | 05/22/2000 |
| SEQ ID NO:6064 | 60/200,779 | 05/22/2000 |
| SEQ ID NO:6065 | 60/200,779 | 05/22/2000 |
| SEQ ID NO:6066 | 60/200,779 | 05/22/2000 |
| SEQ ID NO:6067 | 60/200,779 | 05/22/2000 |
| SEQ ID NO:6068 | 60/200,779 | 05/22/2000 |
| SEQ ID NO:6069 | 60/200,779 | 05/22/2000 |
| SEQ ID NO:6070 | 60/200,779 | 05/22/2000 |
| SEQ ID NO:6071 | 60/200,779 | 05/22/2000 |
| SEQ ID NO:6072 | 60/200,779 | 05/22/2000 |
| SEQ ID NO:6073 | 60/200,779 | 05/22/2000 |
| SEQ ID NO:6074 | 60/200,779 | 05/22/2000 |
| SEQ ID NO:6075 | 60/200,779 | 05/22/2000 |
| SEQ ID NO:6076 | 60/200,779 | 05/22/2000 |
| SEQ ID NO:6077 | 60/200,779 | 05/22/2000 |
| SEQ ID NO:6078 | 60/200,779 | 05/22/2000 |
| SEQ ID NO:6079 | 60/200,779 | 05/22/2000 |
| SEQ ID NO:6080 | 60/200,779 | 05/22/2000 |
| SEQ ID NO:6081 | 60/200,779 | 05/22/2000 |
| SEQ ID NO:6082 | 60/200,779 | 05/22/2000 |
| SEQ ID NO:6083 | 60/200,779 | 05/22/2000 |
| SEQ ID NO:6084 | 60/200,779 | 05/22/2000 |
| SEQ ID NO:6085 | 60/200,779 | 05/22/2000 |
| SEQ ID NO:6086 | 60/200,779 | 05/22/2000 |
| SEQ ID NO:6087 | 60/200,779 | 05/22/2000 |
| SEQ ID NO:6088 | 60/200,779 | 05/22/2000 |
| SEQ ID NO:6089 | 60/200,779 | 05/22/2000 |
| SEQ ID NO:6090 | 60/200,779 | 05/22/2000 |
| SEQ ID NO:6091 | 60/200,779 | 05/22/2000 |
| SEQ ID NO:6092 | 60/200,779 | 05/22/2000 |
| SEQ ID NO:6093 | 60/200,779 | 05/22/2000 |
| SEQ ID NO:6094 | 60/200,779 | 05/22/2000 |
| SEQ ID NO:6095 | 60/200,779 | 05/22/2000 |
| SEQ ID NO:6096 | 60/200,779 | 05/22/2000 |
| SEQ ID NO:6097 | 60/200,779 | 05/22/2000 |
| SEQ ID NO:6098 | 60/200,779 | 05/22/2000 |
| SEQ ID NO:6099 | 60/200,779 | 05/22/2000 |
| SEQ ID NO:6100 | 60/200,779 | 05/22/2000 |
| SEQ ID NO:6101 | 60/200,779 | 05/22/2000 |
| SEQ ID NO:6102 | 60/200,779 | 05/22/2000 |
| SEQ ID NO:6103 | 60/200,779 | 05/22/2000 |
| SEQ ID NO:6104 | 60/200,779 | 05/22/2000 |
| SEQ ID NO:6105 | 60/200,779 | 05/22/2000 |
| SEQ ID NO:6106 | 60/200,779 | 05/22/2000 |
| SEQ ID NO:6107 | 60/200,779 | 05/22/2000 |
| SEQ ID NO:6108 | 60/200,779 | 05/22/2000 |
| SEQ ID NO:6109 | 60/200,779 | 05/22/2000 |
| SEQ ID NO:6110 | 60/200,779 | 05/22/2000 |
| SEQ ID NO:6111 | 60/200,779 | 05/22/2000 |
| SEQ ID NO:6112 | 60/200,779 | 05/22/2000 |
| SEQ ID NO:6113 | 60/200,779 | 05/22/2000 |
| SEQ ID NO:6114 | 60/200,779 | 05/22/2000 |
| SEQ ID NO:6115 | 60/200,779 | 05/22/2000 |
| SEQ ID NO:6116 | 60/200,779 | 05/22/2000 |
| SEQ ID NO:6117 | 60/200,779 | 05/22/2000 |
| SEQ ID NO:6118 | 60/200,779 | 05/22/2000 |
| SEQ ID NO:6119 | 60/200,779 | 05/22/2000 |
| SEQ ID NO:6120 | 60/200,779 | 05/22/2000 |
| SEQ ID NO:6121 | 60/200,779 | 05/22/2000 |
| SEQ ID NO:6122 | 60/200,779 | 05/22/2000 |
| SEQ ID NO:6123 | 60/200,779 | 05/22/2000 |
| SEQ ID NO:6124 | 60/200,779 | 05/22/2000 |
| SEQ ID NO:6125 | 60/200,779 | 05/22/2000 |
| SEQ ID NO:6126 | 60/200,779 | 05/22/2000 |
| SEQ ID NO:6127 | 60/200,779 | 05/22/2000 |
| SEQ ID NO:6128 | 60/200,779 | 05/22/2000 |
| SEQ ID NO:6129 | 60/200,779 | 05/22/2000 |
| SEQ ID NO:6130 | 60/200,779 | 05/22/2000 |
| SEQ ID NO:6131 | 60/200,779 | 05/22/2000 |
| SEQ ID NO:6132 | 60/200,779 | 05/22/2000 |
| SEQ ID NO:6133 | 60/200,779 | 05/22/2000 |
| SEQ ID NO:6134 | 60/200,779 | 05/22/2000 |
| SEQ ID NO:6135 | 60/200,779 | 05/22/2000 |
| SEQ ID NO:6136 | 60/200,779 | 05/22/2000 |
| SEQ ID NO:6137 | 60/200,779 | 05/22/2000 |
| SEQ ID NO:6138 | 60/200,779 | 05/22/2000 |
| SEQ ID NO:6139 | 60/200,779 | 05/22/2000 |
| SEQ ID NO:6140 | 60/200,779 | 05/22/2000 |
| SEQ ID NO:6141 | 60/200,779 | 05/22/2000 |
| SEQ ID NO:6142 | 60/200,779 | 05/22/2000 |
| SEQ ID NO:6143 | 60/200,779 | 05/22/2000 |
| SEQ ID NO:6144 | 60/200,779 | 05/22/2000 |
| SEQ ID NO:6145 | 60/200,779 | 05/22/2000 |
| SEQ ID NO:6146 | 60/200,779 | 05/22/2000 |
| SEQ ID NO:6147 | 60/200,779 | 05/22/2000 |
| SEQ ID NO:6148 | 60/200,779 | 05/22/2000 |
| SEQ ID NO:6149 | 60/200,779 | 05/22/2000 |
| SEQ ID NO:6150 | 60/200,779 | 05/22/2000 |
| SEQ ID NO:6151 | 60/200,779 | 05/22/2000 |
| SEQ ID NO:6152 | 60/200,779 | 05/22/2000 |
| SEQ ID NO:6153 | 60/200,779 | 05/22/2000 |
| SEQ ID NO:6154 | 60/200,779 | 05/22/2000 |
| SEQ ID NO:6155 | 60/200,779 | 05/22/2000 |
| SEQ ID NO:6156 | 60/200,779 | 05/22/2000 |
| SEQ ID NO:6157 | 60/200,779 | 05/22/2000 |
| SEQ ID NO:6158 | 60/200,779 | 05/22/2000 |
| SEQ ID NO:6159 | 60/200,779 | 05/22/2000 |
| SEQ ID NO:6160 | 60/200,779 | 05/22/2000 |
| SEQ ID NO:6161 | 60/200,779 | 05/22/2000 |
| SEQ ID NO:6162 | 60/200,779 | 05/22/2000 |
| SEQ ID NO:6163 | 60/200,779 | 05/22/2000 |
| SEQ ID NO:6164 | 60/200,779 | 05/22/2000 |
| SEQ ID NO:6165 | 60/200,779 | 05/22/2000 |
| SEQ ID NO:6166 | 60/200,779 | 05/22/2000 |
| SEQ ID NO:6167 | 60/200,779 | 05/22/2000 |
| SEQ ID NO:6168 | 60/200,779 | 05/22/2000 |
| SEQ ID NO:6169 | 60/200,779 | 05/22/2000 |
| SEQ ID NO:6170 | 60/200,779 | 05/22/2000 |
| SEQ ID NO:6171 | 60/200,779 | 05/22/2000 |
| SEQ ID NO:6172 | 60/200,779 | 05/22/2000 |
| SEQ ID NO:6173 | 60/200,779 | 05/22/2000 |
| SEQ ID NO:6174 | 60/200,779 | 05/22/2000 |
| SEQ ID NO:6175 | 60/200,779 | 05/22/2000 |
| SEQ ID NO:6176 | 60/200,779 | 05/22/2000 |
| SEQ ID NO:6177 | 60/200,779 | 05/22/2000 |
| SEQ ID NO:6178 | 60/200,779 | 05/22/2000 |
| SEQ ID NO:6179 | 60/200,779 | 05/22/2000 |
| SEQ ID NO:6180 | 60/200,779 | 05/22/2000 |
| SEQ ID NO:6181 | 60/200,779 | 05/22/2000 |
| SEQ ID NO:6182 | 60/200,779 | 05/22/2000 |
| SEQ ID NO:6183 | 60/200,779 | 05/22/2000 |
| SEQ ID NO:6184 | 60/200,779 | 05/22/2000 |
| SEQ ID NO:6185 | 60/200,779 | 05/22/2000 |
| SEQ ID NO:6186 | 60/200,779 | 05/22/2000 |
| SEQ ID NO:6187 | 60/200,779 | 05/22/2000 |
| SEQ ID NO:6188 | 60/200,779 | 05/22/2000 |
| SEQ ID NO:6189 | 60/200,779 | 05/22/2000 |
| SEQ ID NO:6190 | 60/200,779 | 05/22/2000 |
| SEQ ID NO:6191 | 60/200,779 | 05/22/2000 |
| SEQ ID NO:6192 | 60/200,779 | 05/22/2000 |
| SEQ ID NO:6193 | 60/200,779 | 05/22/2000 |
| SEQ ID NO:6194 | 60/200,779 | 05/22/2000 |
| SEQ ID NO:6195 | 60/200,779 | 05/22/2000 |
| SEQ ID NO:6196 | 60/200,779 | 05/22/2000 |
| SEQ ID NO:6197 | 60/200,779 | 05/22/2000 |
| SEQ ID NO:6198 | 60/200,779 | 05/22/2000 |
| SEQ ID NO:6199 | 60/200,779 | 05/22/2000 |
| SEQ ID NO:6200 | 60/200,779 | 05/22/2000 |
| SEQ ID NO:6201 | 60/200,779 | 05/22/2000 |
| SEQ ID NO:6202 | 60/200,999 | 05/01/2000 |
| SEQ ID NO:6203 | 60/200,999 | 05/01/2000 |
| SEQ ID NO:6204 | 60/200,999 | 05/01/2000 |
| SEQ ID NO:6205 | 60/200,999 | 05/01/2000 |
| SEQ ID NO:6206 | 60/200,999 | 05/01/2000 |
| SEQ ID NO:6207 | 60/200,999 | 05/01/2000 |
| SEQ ID NO:6208 | 60/200,999 | 05/01/2000 |
| SEQ ID NO:6209 | 60/200,999 | 05/01/2000 |
| SEQ ID NO:6210 | 60/200,999 | 05/01/2000 |
| SEQ ID NO:6211 | 60/200,999 | 05/01/2000 |
| SEQ ID NO:6212 | 60/200,999 | 05/01/2000 |
| SEQ ID NO:6213 | 60/200,999 | 05/01/2000 |
| SEQ ID NO:6214 | 60/200,999 | 05/01/2000 |
| SEQ ID NO:6215 | 60/200,999 | 05/01/2000 |
| SEQ ID NO:6216 | 60/200,999 | 05/01/2000 |
| SEQ ID NO:6217 | 60/200,999 | 05/01/2000 |
| SEQ ID NO:6218 | 60/200,999 | 05/01/2000 |
| SEQ ID NO:6219 | 60/200,999 | 05/01/2000 |
| SEQ ID NO:6220 | 60/200,999 | 05/01/2000 |
| SEQ ID NO:6221 | 60/200,999 | 05/01/2000 |
| SEQ ID NO:6222 | 60/202,084 | 05/04/2000 |
| SEQ ID NO:6223 | 60/200,545 | 04/27/2000 |
| SEQ ID NO:6224 | 60/200,545 | 04/27/2000 |
| SEQ ID NO:6225 | 60/200,545 | 04/27/2000 |
| SEQ ID NO:6226 | 60/200,545 | 04/27/2000 |
| SEQ ID NO:6227 | 60/200,545 | 04/27/2000 |
| SEQ ID NO:6228 | 60/200,545 | 04/27/2000 |
| SEQ ID NO:6229 | 60/200,545 | 04/27/2000 |
| SEQ ID NO:6230 | 60/200,545 | 04/27/2000 |
| SEQ ID NO:6231 | 60/200,545 | 04/27/2000 |
| SEQ ID NO:6232 | 60/200,545 | 04/27/2000 |
| SEQ ID NO:6233 | 60/200,545 | 04/27/2000 |
| SEQ ID NO:6234 | 60/200,545 | 04/27/2000 |
| SEQ ID NO:6235 | 60/200,545 | 04/27/2000 |
| SEQ ID NO:6236 | 60/200,545 | 04/27/2000 |
| SEQ ID NO:6237 | 60/200,545 | 04/27/2000 |
| SEQ ID NO:6238 | 60/200,545 | 04/27/2000 |
| SEQ ID NO:6239 | 60/200,545 | 04/27/2000 |
| SEQ ID NO:6240 | 60/200,545 | 04/27/2000 |
| SEQ ID NO:6241 | 60/200,545 | 04/27/2000 |
| SEQ ID NO:6242 | 60/200,545 | 04/27/2000 |
| SEQ ID NO:6243 | 60/200,545 | 04/27/2000 |
| SEQ ID NO:6244 | 60/200,545 | 04/27/2000 |
| SEQ ID NO:6245 | 60/200,545 | 04/27/2000 |
| SEQ ID NO:6246 | 60/200,545 | 04/27/2000 |
| SEQ ID NO:6247 | 60/200,545 | 04/27/2000 |
| SEQ ID NO:6248 | 60/200,545 | 04/27/2000 |
| SEQ ID NO:6249 | 60/200,545 | 04/27/2000 |
| SEQ ID NO:6250 | 60/200,545 | 04/27/2000 |
| SEQ ID NO:6251 | 60/200,545 | 04/27/2000 |
| SEQ ID NO:6252 | 60/200,545 | 04/27/2000 |
| SEQ ID NO:6253 | 60/200,545 | 04/27/2000 |
| SEQ ID NO:6254 | 60/200,545 | 04/27/2000 |
| SEQ ID NO:6255 | 60/200,545 | 04/27/2000 |
| SEQ ID NO:6256 | 60/200,545 | 04/27/2000 |
| SEQ ID NO:6257 | 60/200,545 | 04/27/2000 |
| SEQ ID NO:6258 | 60/200,545 | 04/27/2000 |
| SEQ ID NO:6259 | 60/200,545 | 04/27/2000 |
| SEQ ID NO:6260 | 60/200,545 | 04/27/2000 |
| SEQ ID NO:6261 | 60/200,545 | 04/27/2000 |
| SEQ ID NO:6262 | 60/200,545 | 04/27/2000 |
| SEQ ID NO:6263 | 60/200,545 | 04/27/2000 |
| SEQ ID NO:6264 | 60/200,545 | 04/27/2000 |
| SEQ ID NO:6265 | 60/200,545 | 04/27/2000 |
| SEQ ID NO:6266 | 60/200,545 | 04/27/2000 |
| SEQ ID NO:6267 | 60/200,545 | 04/27/2000 |
| SEQ ID NO:6268 | 60/200,545 | 04/27/2000 |
| SEQ ID NO:6269 | 60/200,545 | 04/27/2000 |
| SEQ ID NO:6270 | 60/200,545 | 04/27/2000 |
| SEQ ID NO:6271 | 60/200,545 | 04/27/2000 |
| SEQ ID NO:6272 | 60/200,545 | 04/27/2000 |
| SEQ ID NO:6273 | 60/200,545 | 04/27/2000 |
| SEQ ID NO:6274 | 60/200,545 | 04/27/2000 |
| SEQ ID NO:6275 | 60/200,545 | 04/27/2000 |
| SEQ ID NO:6276 | 60/200,545 | 04/27/2000 |
| SEQ ID NO:6277 | 60/200,545 | 04/27/2000 |
| SEQ ID NO:6278 | 60/200,545 | 04/27/2000 |
| SEQ ID NO:6279 | 60/200,545 | 04/27/2000 |
| SEQ ID NO:6280 | 60/200,545 | 04/27/2000 |
| SEQ ID NO:6281 | 60/200,545 | 04/27/2000 |
| SEQ ID NO:6282 | 60/200,545 | 04/27/2000 |
| SEQ ID NO:6283 | 60/200,545 | 04/27/2000 |
| SEQ ID NO:6284 | 60/200,545 | 04/27/2000 |
| SEQ ID NO:6285 | 60/200,545 | 04/27/2000 |
| SEQ ID NO:6286 | 60/200,545 | 04/27/2000 |
| SEQ ID NO:6287 | 60/200,545 | 04/27/2000 |
| SEQ ID NO:6288 | 60/200,545 | 04/27/2000 |
| SEQ ID NO:6289 | 60/200,545 | 04/27/2000 |
| SEQ ID NO:6290 | 60/200,545 | 04/27/2000 |
| SEQ ID NO:6291 | 60/200,545 | 04/27/2000 |
| SEQ ID NO:6292 | 60/200,545 | 04/27/2000 |
| SEQ ID NO:6293 | 60/200,545 | 04/27/2000 |
| SEQ ID NO:6294 | 60/200,545 | 04/27/2000 |
| SEQ ID NO:6295 | 60/200,545 | 04/27/2000 |
| SEQ ID NO:6296 | 60/200,545 | 04/27/2000 |
| SEQ ID NO:6297 | 60/200,545 | 04/27/2000 |
| SEQ ID NO:6298 | 60/200,545 | 04/27/2000 |
| SEQ ID NO:6299 | 60/200,545 | 04/27/2000 |
| SEQ ID NO:6300 | 60/200,545 | 04/27/2000 |
| SEQ ID NO:6301 | 60/200,545 | 04/27/2000 |
| SEQ ID NO:6302 | 60/200,545 | 04/27/2000 |
| SEQ ID NO:6303 | 60/200,545 | 04/27/2000 |
| SEQ ID NO:6304 | 60/200,545 | 04/27/2000 |
| SEQ ID NO:6305 | 60/200,545 | 04/27/2000 |
| SEQ ID NO:6306 | 60/200,545 | 04/27/2000 |
| SEQ ID NO:6307 | 60/200,545 | 04/27/2000 |
| SEQ ID NO:6308 | 60/200,545 | 04/27/2000 |
| SEQ ID NO:6309 | 60/200,545 | 04/27/2000 |
| SEQ ID NO:6310 | 60/200,545 | 04/27/2000 |
| SEQ ID NO:6311 | 60/200,545 | 04/27/2000 |
| SEQ ID NO:6312 | 60/200,545 | 04/27/2000 |
| SEQ ID NO:6313 | 60/200,545 | 04/27/2000 |
| SEQ ID NO:6314 | 60/200,545 | 04/27/2000 |
| SEQ ID NO:6315 | 60/200,545 | 04/27/2000 |
| SEQ ID NO:6316 | 60/200,545 | 04/27/2000 |
| SEQ ID NO:6317 | 60/200,545 | 04/27/2000 |
| SEQ ID NO:6318 | 60/200,545 | 04/27/2000 |
| SEQ ID NO:6319 | 60/200,545 | 04/27/2000 |
| SEQ ID NO:6320 | 60/200,545 | 04/27/2000 |
| SEQ ID NO:6321 | 60/200,545 | 04/27/2000 |
| SEQ ID NO:6322 | 60/200,545 | 04/27/2000 |
| SEQ ID NO:6323 | 60/200,545 | 04/27/2000 |
| SEQ ID NO:6324 | 60/200,545 | 04/27/2000 |
| SEQ ID NO:6325 | 60/200,545 | 04/27/2000 |
| SEQ ID NO:6326 | 60/200,545 | 04/27/2000 |
| SEQ ID NO:6327 | 60/200,545 | 04/27/2000 |
| SEQ ID NO:6328 | 60/200,545 | 04/27/2000 |
| SEQ ID NO:6329 | 60/200,545 | 04/27/2000 |
| SEQ ID NO:6330 | 60/200,545 | 04/27/2000 |
| SEQ ID NO:6331 | 60/200,545 | 04/27/2000 |
| SEQ ID NO:6332 | 60/200,545 | 04/27/2000 |
| SEQ ID NO:6333 | 60/200,545 | 04/27/2000 |
| SEQ ID NO:6334 | 60/200,545 | 04/27/2000 |
| SEQ ID NO:6335 | 60/200,545 | 04/27/2000 |
| SEQ ID NO:6336 | 60/200,545 | 04/27/2000 |
| SEQ ID NO:6337 | 60/200,545 | 04/27/2000 |
| SEQ ID NO:6338 | 60/200,545 | 04/27/2000 |
| SEQ ID NO:6339 | 60/200,545 | 04/27/2000 |
| SEQ ID NO:6340 | 60/200,545 | 04/27/2000 |
| SEQ ID NO:6341 | 60/200,545 | 04/27/2000 |
| SEQ ID NO:6342 | 60/200,545 | 04/27/2000 |
| SEQ ID NO:6343 | 60/200,545 | 04/27/2000 |
| SEQ ID NO:6344 | 60/200,545 | 04/27/2000 |
| SEQ ID NO:6345 | 60/200,545 | 04/27/2000 |
| SEQ ID NO:6346 | 60/200,545 | 04/27/2000 |
| SEQ ID NO:6347 | 60/200,545 | 04/27/2000 |
| SEQ ID NO:6348 | 60/200,545 | 04/27/2000 |
| SEQ ID NO:6349 | 60/200,545 | 04/27/2000 |
| SEQ ID NO:6350 | 60/200,545 | 04/27/2000 |
| SEQ ID NO:6351 | 60/200,545 | 04/27/2000 |
| SEQ ID NO:6352 | 60/200,545 | 04/27/2000 |
| SEQ ID NO:6353 | 60/200,545 | 04/27/2000 |
| SEQ ID NO:6354 | 60/200,545 | 04/27/2000 |
| SEQ ID NO:6355 | 60/200,545 | 04/27/2000 |
| SEQ ID NO:6356 | 60/200,545 | 04/27/2000 |
| SEQ ID NO:6357 | 60/200,545 | 04/27/2000 |
| SEQ ID NO:6358 | 60/200,545 | 04/27/2000 |
| SEQ ID NO:6359 | 60/200,545 | 04/27/2000 |
| SEQ ID NO:6360 | 60/200,545 | 04/27/2000 |
| SEQ ID NO:6361 | 60/200,545 | 04/27/2000 |
| SEQ ID NO:6362 | 60/200,545 | 04/27/2000 |
| SEQ ID NO:6363 | 60/200,545 | 04/27/2000 |
| SEQ ID NO:6364 | 60/200,545 | 04/27/2000 |
| SEQ ID NO:6365 | 60/200,545 | 04/27/2000 |
| SEQ ID NO:6366 | 60/200,545 | 04/27/2000 |
| SEQ ID NO:6367 | 60/200,545 | 04/27/2000 |
| SEQ ID NO:6368 | 60/200,545 | 04/27/2000 |
| SEQ ID NO:6369 | 60/200,545 | 04/27/2000 |
| SEQ ID NO:6370 | 60/200,545 | 04/27/2000 |
| SEQ ID NO:6371 | 60/200,545 | 04/27/2000 |
| SEQ ID NO:6372 | 60/200,545 | 04/27/2000 |
| SEQ ID NO:6373 | 60/200,545 | 04/27/2000 |
| SEQ ID NO:6374 | 60/200,545 | 04/27/2000 |
| SEQ ID NO:6375 | 60/200,545 | 04/27/2000 |
| SEQ ID NO:6376 | 60/200,545 | 04/27/2000 |
| SEQ ID NO:6377 | 60/200,545 | 04/27/2000 |
| SEQ ID NO:6378 | 60/200,545 | 04/27/2000 |
| SEQ ID NO:6379 | 60/200,545 | 04/27/2000 |
| SEQ ID NO:6380 | 60/200,545 | 04/27/2000 |
| SEQ ID NO:6381 | 60/200,545 | 04/27/2000 |
| SEQ ID NO:6382 | 60/200,545 | 04/27/2000 |
| SEQ ID NO:6383 | 60/200,545 | 04/27/2000 |
| SEQ ID NO:6384 | 60/200,545 | 04/27/2000 |
| SEQ ID NO:6385 | 60/200,545 | 04/27/2000 |
| SEQ ID NO:6386 | 60/200,545 | 04/27/2000 |
| SEQ ID NO:6387 | 60/200,545 | 04/27/2000 |
| SEQ ID NO:6388 | 60/200,545 | 04/27/2000 |
| SEQ ID NO:6389 | 60/200,545 | 04/27/2000 |
| SEQ ID NO:6390 | 60/200,545 | 04/27/2000 |
| SEQ ID NO:6391 | 60/200,545 | 04/27/2000 |
| SEQ ID NO:6392 | 60/200,545 | 04/27/2000 |
| SEQ ID NO:6393 | 60/200,545 | 04/27/2000 |
| SEQ ID NO:6394 | 60/200,545 | 04/27/2000 |
| SEQ ID NO:6395 | 60/200,545 | 04/27/2000 |
| SEQ ID NO:6396 | 60/200,545 | 04/27/2000 |
| SEQ ID NO:6397 | 60/200,545 | 04/27/2000 |
| SEQ ID NO:6398 | 60/200,545 | 04/27/2000 |
| SEQ ID NO:6399 | 60/200,545 | 04/27/2000 |
| SEQ ID NO:6400 | 60/200,545 | 04/27/2000 |
| SEQ ID NO:6401 | 60/200,545 | 04/27/2000 |
| SEQ ID NO:6402 | 60/200,545 | 04/27/2000 |
| SEQ ID NO:6403 | 60/200,545 | 04/27/2000 |
| SEQ ID NO:6404 | 60/200,545 | 04/27/2000 |
| SEQ ID NO:6405 | 60/200,545 | 04/27/2000 |
| SEQ ID NO:6406 | 60/200,545 | 04/27/2000 |
| SEQ ID NO:6407 | 60/200,545 | 04/27/2000 |
| SEQ ID NO:6408 | 60/200,545 | 04/27/2000 |
| SEQ ID NO:6409 | 60/200,545 | 04/27/2000 |
| SEQ ID NO:6410 | 60/200,545 | 04/27/2000 |
| SEQ ID NO:6411 | 60/200,545 | 04/27/2000 |
| SEQ ID NO:6412 | 60/200,545 | 04/27/2000 |
| SEQ ID NO:6413 | 60/200,545 | 04/27/2000 |
| SEQ ID NO:6414 | 60/200,545 | 04/27/2000 |
| SEQ ID NO:6415 | 60/200,545 | 04/27/2000 |
| SEQ ID NO:6416 | 60/200,545 | 04/27/2000 |
| SEQ ID NO:6417 | 60/200,545 | 04/27/2000 |
| SEQ ID NO:6418 | 60/200,545 | 04/27/2000 |
| SEQ ID NO:6419 | 60/200,545 | 04/27/2000 |
| SEQ ID NO:6420 | 60/200,545 | 04/27/2000 |
| SEQ ID NO:6421 | 60/200,545 | 04/27/2000 |
| SEQ ID NO:6422 | 60/200,545 | 04/27/2000 |
| SEQ ID NO:6423 | 60/200,545 | 04/27/2000 |
| SEQ ID NO:6424 | 60/200,545 | 04/27/2000 |
| SEQ ID NO:6425 | 60/200,545 | 04/27/2000 |
| SEQ ID NO:6426 | 60/200,545 | 04/27/2000 |
| SEQ ID NO:6427 | 60/200,545 | 04/27/2000 |
| SEQ ID NO:6428 | 60/200,545 | 04/27/2000 |
| SEQ ID NO:6429 | 60/200,545 | 04/27/2000 |
| SEQ ID NO:6430 | 60/200,545 | 04/27/2000 |
| SEQ ID NO:6431 | 60/200,545 | 04/27/2000 |
| SEQ ID NO:6432 | 60/200,545 | 04/27/2000 |
| SEQ ID NO:6433 | 60/200,545 | 04/27/2000 |
| SEQ ID NO:6434 | 60/200,545 | 04/27/2000 |
| SEQ ID NO:6435 | 60/200,545 | 04/27/2000 |
| SEQ ID NO:6436 | 60/200,545 | 04/27/2000 |
| SEQ ID NO:6437 | 60/200,545 | 04/27/2000 |
| SEQ ID NO:6438 | 60/200,545 | 04/27/2000 |
| SEQ ID NO:6439 | 60/200,545 | 04/27/2000 |
| SEQ ID NO:6440 | 60/200,545 | 04/27/2000 |
| SEQ ID NO:6441 | 60/200,545 | 04/27/2000 |
| SEQ ID NO:6442 | 60/200,545 | 04/27/2000 |
| SEQ ID NO:6443 | 60/200,545 | 04/27/2000 |
| SEQ ID NO:6444 | 60/200,545 | 04/27/2000 |
| SEQ ID NO:6445 | 60/200,545 | 04/27/2000 |
| SEQ ID NO:6446 | 60/200,545 | 04/27/2000 |
| SEQ ID NO:6447 | 60/200,545 | 04/27/2000 |
| SEQ ID NO:6448 | 60/200,545 | 04/27/2000 |
| SEQ ID NO:6449 | 60/200,545 | 04/27/2000 |
| SEQ ID NO:6450 | 60/200,545 | 04/27/2000 |
| SEQ ID NO:6451 | 60/200,545 | 04/27/2000 |
| SEQ ID NO:6452 | 60/200,545 | 04/27/2000 |
| SEQ ID NO:6453 | 60/200,545 | 04/27/2000 |
| SEQ ID NO:6454 | 60/200,545 | 04/27/2000 |
| SEQ ID NO:6455 | 60/200,545 | 04/27/2000 |
| SEQ ID NO:6456 | 60/200,545 | 04/27/2000 |
| SEQ ID NO:6457 | 60/200,545 | 04/27/2000 |
| SEQ ID NO:6458 | 60/200,545 | 04/27/2000 |
| SEQ ID NO:6459 | 60/200,545 | 04/27/2000 |
| SEQ ID NO:6460 | 60/200,545 | 04/27/2000 |
| SEQ ID NO:6461 | 60/200,545 | 04/27/2000 |
| SEQ ID NO:6462 | 60/200,545 | 04/27/2000 |
| SEQ ID NO:6463 | 60/200,545 | 04/27/2000 |
| SEQ ID NO:6464 | 60/200,545 | 04/27/2000 |
| SEQ ID NO:6465 | 60/200,545 | 04/27/2000 |
| SEQ ID NO:6466 | 60/200,545 | 04/27/2000 |
| SEQ ID NO:6467 | 60/200,545 | 04/27/2000 |
| SEQ ID NO:6468 | 60/200,545 | 04/27/2000 |
| SEQ ID NO:6469 | 60/200,545 | 04/27/2000 |
| SEQ ID NO:6470 | 60/200,545 | 04/27/2000 |
| SEQ ID NO:6471 | 60/200,545 | 04/27/2000 |
| SEQ ID NO:6472 | 60/200,545 | 04/27/2000 |
| SEQ ID NO:6473 | 60/200,545 | 04/27/2000 |
| SEQ ID NO:6474 | 60/200,545 | 04/27/2000 |
| SEQ ID NO:6475 | 60/200,545 | 04/27/2000 |
| SEQ ID NO:6476 | 60/200,545 | 04/27/2000 |
| SEQ ID NO:6477 | 60/200,545 | 04/27/2000 |
| SEQ ID NO:6478 | 60/200,545 | 04/27/2000 |
| SEQ ID NO:6479 | 60/200,545 | 04/27/2000 |
| SEQ ID NO:6480 | 60/200,545 | 04/27/2000 |
| SEQ ID NO:6481 | 60/200,545 | 04/27/2000 |
| SEQ ID NO:6482 | 60/200,545 | 04/27/2000 |
| SEQ ID NO:6483 | 60/200,545 | 04/27/2000 |
| SEQ ID NO:6484 | 60/200,545 | 04/27/2000 |
| SEQ ID NO:6485 | 60/200,545 | 04/27/2000 |
| SEQ ID NO:6486 | 60/200,545 | 04/27/2000 |
| SEQ ID NO:6487 | 60/200,545 | 04/27/2000 |
| SEQ ID NO:6488 | 60/200,545 | 04/27/2000 |
| SEQ ID NO:6489 | 60/200,545 | 04/27/2000 |
| SEQ ID NO:6490 | 60/200,545 | 04/27/2000 |
| SEQ ID NO:6491 | 60/200,545 | 04/27/2000 |
| SEQ ID NO:6492 | 60/200,545 | 04/27/2000 |
| SEQ ID NO:6493 | 60/200,545 | 04/27/2000 |
| SEQ ID NO:6494 | 60/200,545 | 04/27/2000 |
| SEQ ID NO:6495 | 60/200,545 | 04/27/2000 |
| SEQ ID NO:6496 | 60/200,545 | 04/27/2000 |
| SEQ ID NO:6497 | 60/200,545 | 04/27/2000 |
| SEQ ID NO:6498 | 60/200,545 | 04/27/2000 |
| SEQ ID NO:6499 | 60/200,545 | 04/27/2000 |
| SEQ ID NO:6500 | 60/200,545 | 04/27/2000 |
| SEQ ID NO:6501 | 60/200,545 | 04/27/2000 |
| SEQ ID NO:6502 | 60/200,545 | 04/27/2000 |
| SEQ ID NO:6503 | 60/200,545 | 04/27/2000 |
| SEQ ID NO:6504 | 60/200,545 | 04/27/2000 |
| SEQ ID NO:6505 | 60/200,545 | 04/27/2000 |
| SEQ ID NO:6506 | 60/200,545 | 04/27/2000 |
| SEQ ID NO:6507 | 60/200,545 | 04/27/2000 |
| SEQ ID NO:6508 | 60/200,545 | 04/27/2000 |
| SEQ ID NO:6509 | 60/200,545 | 04/27/2000 |
| SEQ ID NO:6510 | 60/200,545 | 04/27/2000 |
| SEQ ID NO:6511 | 60/200,545 | 04/27/2000 |
| SEQ ID NO:6512 | 60/200,545 | 04/27/2000 |
| SEQ ID NO:6513 | 60/200,545 | 04/27/2000 |
| SEQ ID NO:6514 | 60/200,545 | 04/27/2000 |
| SEQ ID NO:6515 | 60/200,545 | 04/27/2000 |
| SEQ ID NO:6516 | 60/200,545 | 04/27/2000 |
| SEQ ID NO:6517 | 60/200,545 | 04/27/2000 |
| SEQ ID NO:6518 | 60/200,545 | 04/27/2000 |
| SEQ ID NO:6519 | 60/200,545 | 04/27/2000 |
| SEQ ID NO:6520 | 60/200,545 | 04/27/2000 |
| SEQ ID NO:6521 | 60/200,545 | 04/27/2000 |
| SEQ ID NO:6522 | 60/200,545 | 04/27/2000 |
| SEQ ID NO:6523 | 60/200,545 | 04/27/2000 |
| SEQ ID NO:6524 | 60/200,545 | 04/27/2000 |
| SEQ ID NO:6525 | 60/200,545 | 04/27/2000 |
| SEQ ID NO:6526 | 60/200,545 | 04/27/2000 |
| SEQ ID NO:6527 | 60/200,545 | 04/27/2000 |
| SEQ ID NO:6528 | 60/200,545 | 04/27/2000 |
| SEQ ID NO:6529 | 60/200,545 | 04/27/2000 |
| SEQ ID NO:6530 | 60/200,545 | 04/27/2000 |
| SEQ ID NO:6531 | 60/200,545 | 04/27/2000 |
| SEQ ID NO:6532 | 60/200,545 | 04/27/2000 |
| SEQ ID NO:6533 | 60/200,545 | 04/27/2000 |
| SEQ ID NO:6534 | 60/200,545 | 04/27/2000 |
| SEQ ID NO:6535 | 60/200,545 | 04/27/2000 |
| SEQ ID NO:6536 | 60/200,545 | 04/27/2000 |
| SEQ ID NO:6537 | 60/200,545 | 04/27/2000 |
| SEQ ID NO:6538 | 60/200,545 | 04/27/2000 |
| SEQ ID NO:6539 | 60/200,545 | 04/27/2000 |
| SEQ ID NO:6540 | 60/200,545 | 04/27/2000 |
| SEQ ID NO:6541 | 60/200,545 | 04/27/2000 |
| SEQ ID NO:6542 | 60/200,545 | 04/27/2000 |
| SEQ ID NO:6543 | 60/200,545 | 04/27/2000 |
| SEQ ID NO:6544 | 60/200,545 | 04/27/2000 |
| SEQ ID NO:6545 | 60/200,545 | 04/27/2000 |
| SEQ ID NO:6546 | 60/200,545 | 04/27/2000 |
| SEQ ID NO:6547 | 60/200,545 | 04/27/2000 |
| SEQ ID NO:6548 | 60/200,545 | 04/27/2000 |
| SEQ ID NO:6549 | 60/200,545 | 04/27/2000 |
| SEQ ID NO:6550 | 60/200,545 | 04/27/2000 |
| SEQ ID NO:6551 | 60/200,545 | 04/27/2000 |
| SEQ ID NO:6552 | 60/200,545 | 04/27/2000 |
| SEQ ID NO:6553 | 60/200,545 | 04/27/2000 |
| SEQ ID NO:6554 | 60/200,545 | 04/27/2000 |
| SEQ ID NO:6555 | 60/200,545 | 04/27/2000 |
| SEQ ID NO:6556 | 60/200,545 | 04/27/2000 |
| SEQ ID NO:6557 | 60/200,545 | 04/27/2000 |
| SEQ ID NO:6558 | 60/200,545 | 04/27/2000 |
| SEQ ID NO:6559 | 60/200,545 | 04/27/2000 |
| SEQ ID NO:6560 | 60/200,545 | 04/27/2000 |
| SEQ ID NO:6561 | 60/200,545 | 04/27/2000 |
| SEQ ID NO:6562 | 60/200,545 | 04/27/2000 |
| SEQ ID NO:6563 | 60/200,545 | 04/27/2000 |
| SEQ ID NO:6564 | 60/200,545 | 04/27/2000 |
| SEQ ID NO:6565 | 60/200,545 | 04/27/2000 |
| SEQ ID NO:6566 | 60/200,545 | 04/27/2000 |
| SEQ ID NO:6567 | 60/200,545 | 04/27/2000 |
| SEQ ID NO:6568 | 60/200,545 | 04/27/2000 |
| SEQ ID NO:6569 | 60/200,545 | 04/27/2000 |
| SEQ ID NO:6570 | 60/200,545 | 04/27/2000 |
| SEQ ID NO:6571 | 60/200,545 | 04/27/2000 |
| SEQ ID NO:6572 | 60/200,545 | 04/27/2000 |
| SEQ ID NO:6573 | 60/200,545 | 04/27/2000 |
| SEQ ID NO:6574 | 60/200,545 | 04/27/2000 |
| SEQ ID NO:6575 | 60/200,545 | 04/27/2000 |
| SEQ ID NO:6576 | 60/200,545 | 04/27/2000 |
| SEQ ID NO:6577 | 60/200,545 | 04/27/2000 |
| SEQ ID NO:6578 | 60/200,545 | 04/27/2000 |
| SEQ ID NO:6579 | 60/200,545 | 04/27/2000 |
| SEQ ID NO:6580 | 60/200,545 | 04/27/2000 |
| SEQ ID NO:658l | 60/200,545 | 04/27/2000 |
| SEQ ID NO:6582 | 60/200,545 | 04/27/2000 |
| SEQ ID NO:6583 | 60/200,545 | 04/27/2000 |
| SEQ ID NO:6584 | 60/200,545 | 04/27/2000 |
| SEQ ID NO:6585 | 60/200,545 | 04/27/2000 |
| SEQ ID NO:6586 | 60/200,545 | 04/27/2000 |
| SEQ ID NO:6587 | 60/200,545 | 04/27/2000 |
| SEQ ID NO:6588 | 60/200,545 | 04/27/2000 |
| SEQ ID NO:6589 | 60/200,545 | 04/27/2000 |
| SEQ ID NO:6590 | 60/200,545 | 04/27/2000 |
| SEQ ID NO:6591 | 60/200,545 | 04/27/2000 |
| SEQ ID NO:6592 | 60/200,545 | 04/27/2000 |
| SEQ ID NO:6593 | 60/200,545 | 04/27/2000 |
| SEQ ID NO:6594 | 60/200,545 | 04/27/2000 |
| SEQ ID NO:6595 | 60/200,545 | 04/27/2000 |
| SEQ ID NO:6596 | 60/200,545 | 04/27/2000 |
| SEQ ID NO:6597 | 60/200,545 | 04/27/2000 |
| SEQ ID NO:6598 | 60/200,545 | 04/27/2000 |
| SEQ ID NO:6599 | 60/200,545 | 04/27/2000 |
| SEQ ID NO:6600 | 60/200,545 | 04/27/2000 |
| SEQ ID NO:6601 | 60/200,545 | 04/27/2000 |
| SEQ ID NO:6602 | 60/200,545 | 04/27/2000 |
| SEQ ID NO:6603 | 60/200,545 | 04/27/2000 |
| SEQ ID NO:6604 | 60/200,545 | 04/27/2000 |
| SEQ ID NO:6605 | 60/200,545 | 04/27/2000 |
| SEQ ID NO:6606 | 60/200,545 | 04/27/2000 |
| SEQ ID NO:6607 | 60/200,545 | 04/27/2000 |
| SEQ ID NO:6608 | 60/200,545 | 04/27/2000 |
| SEQ ID NO:6609 | 60/200,545 | 04/27/2000 |
| SEQ ID NO:6610 | 60/200,545 | 04/27/2000 |
| SEQ ID NO:6611 | 60/200,545 | 04/27/2000 |
| SEQ ID NO:6612 | 60/200,545 | 04/27/2000 |
| SEQ ID NO:6613 | 60/200,545 | 04/27/2000 |
| SEQ ID NO:6614 | 60/200,545 | 04/27/2000 |
| SEQ ID NO:6615 | 60/200,545 | 04/27/2000 |
| SEQ ID NO:6616 | 60/200,545 | 04/27/2000 |
| SEQ ID NO:6617 | 60/200,545 | 04/27/2000 |
| SEQ ID NO:6618 | 60/200,545 | 04/27/2000 |
| SEQ ID NO:6619 | 60/200,545 | 04/27/2000 |
| SEQ ID NO:6620 | 60/200,545 | 04/27/2000 |
| SEQ ID NO:6621 | 60/200,545 | 04/27/2000 |
| SEQ ID NO:6622 | 60/200,545 | 04/27/2000 |
| SEQ ID NO:6623 | 60/200,545 | 04/27/2000 |
| SEQ ID NO:6624 | 60/200,545 | 04/27/2000 |
| SEQ ID NO:6625 | 60/200,545 | 04/27/2000 |
| SEQ ID NO:6626 | 60/200,545 | 04/27/2000 |
| SEQ ID NO:6627 | 60/200,545 | 04/27/2000 |
| SEQ ID NO:6628 | 60/200,545 | 04/27/2000 |
| SEQ ID NO:6629 | 60/200,545 | 04/27/2000 |
| SEQ ID NO:6630 | 60/200,545 | 04/27/2000 |
| SEQ ID NO:6631 | 60/200,545 | 04/27/2000 |
| SEQ ID NO:6632 | 60/200,545 | 04/27/2000 |
| SEQ ID NO:6633 | 60/200,545 | 04/27/2000 |
| SEQ ID NO:6634 | 60/200,545 | 04/27/2000 |
| SEQ ID NO:6635 | 60/200,545 | 04/27/2000 |
| SEQ ID NO:6636 | 60/200,545 | 04/27/2000 |
| SEQ ID NO:6637 | 60/200,545 | 04/27/2000 |
| SEQ ID NO:6638 | 60/200,545 | 04/27/2000 |
| SEQ ID NO:6639 | 60/200,545 | 04/27/2000 |
| SEQ ID NO:6640 | 60/200,545 | 04/27/2000 |
| SEQ ID NO:6641 | 60/200,545 | 04/27/2000 |
| SEQ ID NO:6642 | 60/200,545 | 04/27/2000 |
| SEQ ID NO:6643 | 60/200,545 | 04/27/2000 |
| SEQ ID NO:6644 | 60/200,545 | 04/27/2000 |
| SEQ ID NO:6645 | 60/200,545 | 04/27/2000 |
| SEQ ID NO:6646 | 60/200,545 | 04/27/2000 |
| SEQ ID NO:6647 | 60/200,545 | 04/27/2000 |
| SEQ ID NO:6648 | 60/200,545 | 04/27/2000 |
| SEQ ID NO:6649 | 60/200,545 | 04/27/2000 |
| SEQ ID NO:6650 | 60/200,545 | 04/27/2000 |
| SEQ ID NO:6651 | 60/200,545 | 04/27/2000 |
| SEQ ID NO:6652 | 60/200,545 | 04/27/2000 |
| SEQ ID NO:6653 | 60/200,545 | 04/27/2000 |
| SEQ ID NO:6654 | 60/200,545 | 04/27/2000 |
| SEQ ID NO:6655 | 60/200,545 | 04/27/2000 |
| SEQ ID NO:6656 | 60/200,545 | 04/27/2000 |
| SEQ ID NO:6657 | 60/200,545 | 04/27/2000 |
| SEQ ID NO:6658 | 60/200,545 | 04/27/2000 |
| SEQ ID NO:6659 | 60/200,545 | 04/27/2000 |
| SEQ ID NO:6660 | 60/200,545 | 04/27/2000 |
| SEQ ID NO:6661 | 60/200,545 | 04/27/2000 |
| SEQ ID NO:6662 | 60/200,545 | 04/27/2000 |
| SEQ ID NO:6663 | 60/200,545 | 04/27/2000 |
| SEQ ID NO:6664 | 60/200,545 | 04/27/2000 |
| SEQ ID NO:6665 | 60/200,545 | 04/27/2000 |
| SEQ ID NO:6666 | 60/200,545 | 04/27/2000 |
| SEQ ID NO:6667 | 60/200,545 | 04/27/2000 |
| SEQ ID NO:6668 | 60/200,545 | 04/27/2000 |
| SEQ ID NO:6669 | 60/200,545 | 04/27/2000 |
| SEQ ID NO:6670 | 60/200,545 | 04/27/2000 |
| SEQ ID NO:6671 | 60/200,545 | 04/27/2000 |
| SEQ ID NO:6672 | 60/200,545 | 04/27/2000 |
| SEQ ID NO:6673 | 60/200,545 | 04/27/2000 |
| SEQ ID NO:6674 | 60/200,545 | 04/27/2000 |
| SEQ ID NO:6675 | 60/200,545 | 04/27/2000 |
| SEQ ID NO:6676 | 60/200,545 | 04/27/2000 |
| SEQ ID NO:6677 | 60/200,545 | 04/27/2000 |
| SEQ ID NO:6678 | 60/200,545 | 04/27/2000 |
| SEQ ID NO:6679 | 60/200,545 | 04/27/2000 |
| SEQ ID NO:6680 | 60/200,545 | 04/27/2000 |
| SEQ ID NO:6681 | 60/200,545 | 04/27/2000 |
| SEQ ID NO:6682 | 60/200,545 | 04/27/2000 |
| SEQ ID NO:6683 | 60/200,545 | 04/27/2000 |
| SEQ ID NO:6684 | 60/200,545 | 04/27/2000 |
| SEQ ID NO:6685 | 60/200,545 | 04/27/2000 |
| SEQ ID NO:6686 | 60/200,545 | 04/27/2000 |
| SEQ ID NO:6687 | 60/200,545 | 04/27/2000 |
| SEQ ID NO:6688 | 60/200,545 | 04/27/2000 |
| SEQ ID NO:6689 | 60/200,545 | 04/27/2000 |
| SEQ ID NO:6690 | 60/206,201 | 05/22/2000 |
| SEQ ID NO:6691 | 60/206,201 | 05/22/2000 |
| SEQ ID NO:6692 | 60/206,201 | 05/22/2000 |
| SEQ ID NO:6693 | 60/206,201 | 05/22/2000 |
| SEQ ID NO:6694 | 60/206,201 | 05/22/2000 |
| SEQ ID NO:6695 | 60/206,201 | 05/22/2000 |
| SEQ ID NO:6696 | 60/206,201 | 05/22/2000 |
| SEQ ID NO:6697 | 60/206,201 | 05/22/2000 |
| SEQ ID NO:6698 | 60/206,201 | 05/22/2000 |
| SEQ ID NO:6699 | 60/206,201 | 05/22/2000 |
| SEQ ID NO:6700 | 60/206,201 | 05/22/2000 |
| SEQ ID NO:6701 | 60/206,201 | 05/22/2000 |
| SEQ ID NO:6702 | 60/206,201 | 05/22/2000 |
| SEQ ID NO:6703 | 60/206,201 | 05/22/2000 |
| SEQ ID NO:6704 | 60/206,201 | 05/22/2000 |
| SEQ ID NO:6705 | 60/206,201 | 05/22/2000 |
| SEQ ID NO:6706 | 60/206,201 | 05/22/2000 |
| SEQ ID NO:6707 | 60/206,201 | 05/22/2000 |
| SEQ ID NO:6708 | 60/206,201 | 05/22/2000 |
| SEQ ID NO:6709 | 60/206,201 | 05/22/2000 |
| SEQ ID NO:6710 | 60/206,201 | 05/22/2000 |
| SEQ ID NO:6711 | 60/206,201 | 05/22/2000 |
| SEQ ID NO:6712 | 60/206,201 | 05/22/2000 |
| SEQ ID NO:6713 | 60/206,201 | 05/22/2000 |
| SEQ ID NO:6714 | 60/206,201 | 05/22/2000 |
| SEQ ID NO:6715 | 60/206,201 | 05/22/2000 |
| SEQ ID NO:6716 | 60/206,201 | 05/22/2000 |
| SEQ ID NO:6717 | 60/206,201 | 05/22/2000 |
| SEQ ID NO:6718 | 60/206,201 | 05/22/2000 |
| SEQ ID NO:6719 | 60/206,201 | 05/22/2000 |
| SEQ ID NO:6720 | 60/206,201 | 05/22/2000 |
| SEQ ID NO:6721 | 60/206,201 | 05/22/2000 |
| SEQ ID NO:6722 | 60/206,201 | 05/22/2000 |
| SEQ ID NO:6723 | 60/206,201 | 05/22/2000 |
| SEQ ID NO:6724 | 60/206,201 | 05/22/2000 |
| SEQ ID NO:6725 | 60/206,201 | 05/22/2000 |
| SEQ ID NO:6726 | 60/206,201 | 05/22/2000 |
| SEQ ID NO:6727 | 60/206,201 | 05/22/2000 |
| SEQ ID NO:6728 | 60/206,201 | 05/22/2000 |
| SEQ ID NO:6729 | 60/206,201 | 05/22/2000 |
| SEQ ID NO:6730 | 60/206,201 | 05/22/2000 |
| SEQ ID NO:6731 | 60/206,201 | 05/22/2000 |
| SEQ ID NO:6732 | 60/206,201 | 05/22/2000 |
| SEQ ID NO:6733 | 60/206,201 | 05/22/2000 |
| SEQ ID NO:6734 | 60/206,201 | 05/22/2000 |
| SEQ ID NO:6735 | 60/206,201 | 05/22/2000 |
| SEQ ID NO:6736 | 60/206,201 | 05/22/2000 |
| SEQ ID NO:6737 | 60/206,201 | 05/22/2000 |
| SEQ ID NO:6738 | 60/206,201 | 05/22/2000 |
| SEQ ID NO:6739 | 60/206,201 | 05/22/2000 |
| SEQ ID NO:6740 | 60/206,201 | 05/22/2000 |
| SEQ ID NO:6741 | 60/206,201 | 05/22/2000 |
| SEQ ID NO:6742 | 60/206,201 | 05/22/2000 |
| SEQ ID NO:6743 | 60/206,201 | 05/22/2000 |
| SEQ ID NO:6744 | 60/206,201 | 05/22/2000 |
| SEQ ID NO:6745 | 60/206,201 | 05/22/2000 |
| SEQ ID NO:6746 | 60/206,201 | 05/22/2000 |
| SEQ ID NO:6747 | 60/206,201 | 05/22/2000 |
| SEQ ID NO:6748 | 60/206,201 | 05/22/2000 |
| SEQ ID NO:6749 | 60/206,201 | 05/22/2000 |
| SEQ ID NO:6750 | 60/206,201 | 05/22/2000 |
| SEQ ID NO:6751 | 60/206,201 | 05/22/2000 |
| SEQ ID NO:6752 | 60/206,201 | 05/22/2000 |
| SEQ ID NO:6753 | 60/206,201 | 05/22/2000 |
| SEQ ID NO:6754 | 60/206,201 | 05/22/2000 |
| SEQ ID NO:6755 | 60/206,201 | 05/22/2000 |
| SEQ ID NO:6756 | 60/206,201 | 05/22/2000 |
| SEQ ID NO:6757 | 60/206,201 | 05/22/2000 |
| SEQ ID NO:6758 | 60/206,201 | 05/22/2000 |
| SEQ ID NO:6759 | 60/206,201 | 05/22/2000 |
| SEQ ID NO:6760 | 60/206,201 | 05/22/2000 |
| SEQ ID NO:6761 | 60/206,201 | 05/22/2000 |
| SEQ ID NO:6762 | 60/206,201 | 05/22/2000 |
| SEQ ID NO:6763 | 60/206,201 | 05/22/2000 |
| SEQ ID NO:6764 | 60/206,201 | 05/22/2000 |
| SEQ ID NO:6765 | 60/206,201 | 05/22/2000 |
| SEQ ID NO:6766 | 60/206,201 | 05/22/2000 |
| SEQ ID NO:6767 | 60/206,201 | 05/22/2000 |
| SEQ ID NO:6768 | 60/206,201 | 05/22/2000 |
| SEQ ID NO:6769 | 60/206,201 | 05/22/2000 |
| SEQ ID NO:6770 | 60/206,201 | 05/22/2000 |
| SEQ ID NO:6771 | 60/206,201 | 05/22/2000 |
| SEQ ID NO:6772 | 60/206,201 | 05/22/2000 |
| SEQ ID NO:6773 | 60/206,201 | 05/22/2000 |
| SEQ ID NO:6774 | 60/206,201 | 05/22/2000 |
| SEQ ID NO:6775 | 60/206,201 | 05/22/2000 |
| SEQ ID NO:6776 | 60/206,201 | 05/22/2000 |
| SEQ ID NO:6777 | 60/206,201 | 05/22/2000 |
| SEQ ID NO:6778 | 60/206,201 | 05/22/2000 |
| SEQ ID NO:6779 | 60/206,201 | 05/22/2000 |
| SEQ ID NO:6780 | 60/206,201 | 05/22/2000 |
| SEQ ID NO:6781 | 60/206,201 | 05/22/2000 |
| SEQ ID NO:6782 | 60/206,201 | 05/22/2000 |
| SEQ ID NO:6783 | 60/206,201 | 05/22/2000 |
| SEQ ID NO:6784 | 60/206,201 | 05/22/2000 |
| SEQ ID NO:6785 | 60/206,201 | 05/22/2000 |
| SEQ ID NO:6786 | 60/206,201 | 05/22/2000 |
| SEQ ID NO:6787 | 60/206,201 | 05/22/2000 |
| SEQ ID NO:6788 | 60/206,201 | 05/22/2000 |
| SEQ ID NO:6789 | 60/206,201 | 05/22/2000 |
| SEQ ID NO:6790 | 60/206,201 | 05/22/2000 |
| SEQ ID NO:6791 | 60/206,201 | 05/22/2000 |
| SEQ ID NO:6792 | 60/206,201 | 05/22/2000 |
| SEQ ID NO:6793 | 60/206,201 | 05/22/2000 |
| SEQ ID NO:6794 | 60/206,201 | 05/22/2000 |
| SEQ ID NO:6795 | 60/206,201 | 05/22/2000 |
| SEQ ID NO:6796 | 60/206,201 | 05/22/2000 |
| SEQ ID NO:6797 | 60/206,201 | 05/22/2000 |
| SEQ ID NO:6798 | 60/206,201 | 05/22/2000 |
| SEQ ID NO:6799 | 60/206,201 | 05/22/2000 |
| SEQ ID NO:6800 | 60/206,201 | 05/22/2000 |
| SEQ ID NO:6801 | 60/206,201 | 05/22/2000 |
| SEQ ID NO:6802 | 60/206,201 | 05/22/2000 |
| SEQ ID NO:6803 | 60/206,201 | 05/22/2000 |
| SEQ ID NO:6804 | 60/206,201 | 05/22/2000 |
| SEQ ID NO:6805 | 60/206,201 | 05/22/2000 |
| SEQ ID NO:6806 | 60/206,201 | 05/22/2000 |
| SEQ ID NO:6807 | 60/206,201 | 05/22/2000 |
| SEQ ID NO:6808 | 60/206,201 | 05/22/2000 |
| SEQ ID NO:6809 | 60/206,201 | 05/22/2000 |
| SEQ ID NO:6810 | 60/206,201 | 05/22/2000 |
| SEQ ID NO:6811 | 60/206,201 | 05/22/2000 |
| SEQ ID NO:6812 | 60/206,201 | 05/22/2000 |
| SEQ ID NO:6813 | 60/206,201 | 05/22/2000 |
| SEQ ID NO:6814 | 60/206,201 | 05/22/2000 |
| SEQ ID NO:6815 | 60/206,201 | 05/22/2000 |
| SEQ ID NO:6816 | 60/206,201 | 05/22/2000 |
| SEQ ID NO:6817 | 60/206,201 | 05/22/2000 |
| SEQ ID NO:6818 | 60/206,201 | 05/22/2000 |
| SEQ ID NO:6819 | 60/206,201 | 05/22/2000 |
| SEQ ID NO:6820 | 60/206,201 | 05/22/2000 |
| SEQ ID NO:6821 | 60/206,201 | 05/22/2000 |
| SEQ ID NO:6822 | 60/206,201 | 05/22/2000 |
| SEQ ID NO:6823 | 60/206,201 | 05/22/2000 |
| SEQ ID NO:6824 | 60/206,201 | 05/22/2000 |
| SEQ ID NO:6825 | 60/206,201 | 05/22/2000 |
| SEQ ID NO:6826 | 60/206,201 | 05/22/2000 |
| SEQ ID NO:6827 | 60/206,201 | 05/22/2000 |
| SEQ ID NO:6828 | 60/206,201 | 05/22/2000 |
| SEQ ID NO:6829 | 60/206,201 | 05/22/2000 |
| SEQ ID NO:6830 | 60/206,201 | 05/22/2000 |
| SEQ ID NO:6831 | 60/206,201 | 05/22/2000 |
| SEQ ID NO:6832 | 60/206,201 | 05/22/2000 |
| SEQ ID NO:6833 | 60/206,201 | 05/22/2000 |
| SEQ ID NO:6834 | 60/206,201 | 05/22/2000 |
| SEQ ID NO:6835 | 60/206,201 | 05/22/2000 |
| SEQ ID NO:6836 | 60/206,201 | 05/22/2000 |
| SEQ ID NO:6837 | 60/206,201 | 05/22/2000 |
| SEQ ID NO:6838 | 60/206,201 | 05/22/2000 |
| SEQ ID NO:6839 | 60/206,201 | 05/22/2000 |
| SEQ ID NO:6840 | 60/206,201 | 05/22/2000 |
| SEQ ID NO:6841 | 60/206,201 | 05/22/2000 |
| SEQ ID NO:6842 | 60/206,201 | 05/22/2000 |
| SEQ ID NO:6843 | 60/206,201 | 05/22/2000 |
| SEQ ID NO:6844 | 60/206,201 | 05/22/2000 |
| SEQ ID NO:6845 | 60/206,201 | 05/22/2000 |
| SEQ ID NO:6846 | 60/206,201 | 05/22/2000 |
| SEQ ID NO:6847 | 60/206,201 | 05/22/2000 |
| SEQ ID NO:6848 | 60/206,201 | 05/22/2000 |
| SEQ ID NO:6849 | 60/206,201 | 05/22/2000 |
| SEQ ID NO:6850 | 60/206,201 | 05/22/2000 |
| SEQ ID NO:6851 | 60/206,201 | 05/22/2000 |
| SEQ ID NO:6852 | 60/206,201 | 05/22/2000 |
| SEQ ID NO:6853 | 60/206,201 | 05/22/2000 |
| SEQ ID NO:6854 | 60/206,201 | 05/22/2000 |
| SEQ ID NO:6855 | 60/206,201 | 05/22/2000 |
| SEQ ID NO:6856 | 60/206,201 | 05/22/2000 |
| SEQ ID NO:6857 | 60/206,201 | 05/22/2000 |
| SEQ ID NO:6858 | 60/206,201 | 05/22/2000 |
| SEQ ID NO:6859 | 60/206,201 | 05/22/2000 |
| SEQ ID NO:6860 | 60/206,201 | 05/22/2000 |
| SEQ ID NO:6861 | 60/206,201 | 05/22/2000 |
| SEQ ID NO:6862 | 60/206,201 | 05/22/2000 |
| SEQ ID NO:6863 | 60/206,201 | 05/22/2000 |
| SEQ ID NO:6864 | 60/206,201 | 05/22/2000 |
| SEQ ID NO:6865 | 60/206,201 | 05/22/2000 |
| SEQ ID NO:6866 | 60/206,201 | 05/22/2000 |
| SEQ ID NO:6867 | 60/206,201 | 05/22/2000 |
| SEQ ID NO:6868 | 60/206,201 | 05/22/2000 |
| SEQ ID NO:6869 | 60/206,201 | 05/22/2000 |
| SEQ ID NO:6870 | 60/206,201 | 05/22/2000 |
| SEQ ID NO:6871 | 60/206,201 | 05/22/2000 |
| SEQ ID NO:6872 | 60/206,201 | 05/22/2000 |
| SEQ ID NO:6873 | 60/206,201 | 05/22/2000 |
| SEQ ID NO:6874 | 60/206,201 | 05/22/2000 |
| SEQ ID NO:6875 | 60/206,201 | 05/22/2000 |
| SEQ ID NO:6876 | 60/206,201 | 05/22/2000 |
| SEQ ID NO:6877 | 60/206,201 | 05/22/2000 |
| SEQ ID NO:6878 | 60/206,201 | 05/22/2000 |
| SEQ ID NO:6879 | 60/206,201 | 05/22/2000 |
| SEQ ID NO:6880 | 60/206,201 | 05/22/2000 |
| SEQ ID NO:6881 | 60/206,201 | 05/22/2000 |
| SEQ ID NO:6882 | 60/206,201 | 05/22/2000 |
| SEQ ID NO:6883 | 60/206,201 | 05/22/2000 |
| SEQ ID NO:6884 | 60/206,201 | 05/22/2000 |
| SEQ ID NO:6885 | 60/206,201 | 05/22/2000 |
| SEQ ID NO:6886 | 60/206,201 | 05/22/2000 |
| SEQ ID NO:6887 | 60/206,201 | 05/22/2000 |
| SEQ ID NO:6888 | 60/206,201 | 05/22/2000 |
| SEQ ID NO:6889 | 60/206,201 | 05/22/2000 |
| SEQ ID NO:6890 | 60/206,201 | 05/22/2000 |
| SEQ ID NO:6891 | 60/206,201 | 05/22/2000 |
| SEQ ID NO:6892 | 60/206,201 | 05/22/2000 |
| SEQ ID NO:6893 | 60/206,201 | 05/22/2000 |
| SEQ ID NO:6894 | 60/206,201 | 05/22/2000 |
| SEQ ID NO:6895 | 60/206,201 | 05/22/2000 |
| SEQ ID NO:6896 | 60/206,201 | 05/22/2000 |
| SEQ ID NO:6897 | 60/206,201 | 05/22/2000 |
| SEQ ID NO:6898 | 60/206,201 | 05/22/2000 |
| SEQ ID NO:6899 | 60/206,201 | 05/22/2000 |
| SEQ ID NO:6900 | 60/206,201 | 05/22/2000 |
| SEQ ID NO:6901 | 60/206,201 | 05/22/2000 |
| SEQ ID NO:6902 | 60/206,201 | 05/22/2000 |
| SEQ ID NO:6903 | 60/206,201 | 05/22/2000 |
| SEQ ID NO:6904 | 60/206,201 | 05/22/2000 |
| SEQ ID NO:6905 | 60/206,201 | 05/22/2000 |
| SEQ ID NO:6906 | 60/206,201 | 05/22/2000 |
| SEQ ID NO:6907 | 60/206,201 | 05/22/2000 |
| SEQ ID NO:6908 | 60/206,201 | 05/22/2000 |
| SEQ ID NO:6909 | 60/206,201 | 05/22/2000 |
| SEQ ID NO:6910 | 60/206,201 | 05/22/2000 |
| SEQ ID NO:6911 | 60/206,201 | 05/22/2000 |
| SEQ ID NO:6912 | 60/206,201 | 05/22/2000 |
| SEQ ID NO:6913 | 60/206,201 | 05/22/2000 |
| SEQ ID NO:6914 | 60/206,201 | 05/22/2000 |
| SEQ ID NO:6915 | 60/206,201 | 05/22/2000 |
| SEQ ID NO:6916 | 60/206,201 | 05/22/2000 |
| SEQ ID NO:6917 | 60/206,201 | 05/22/2000 |
| SEQ ID NO:6918 | 60/206,201 | 05/22/2000 |
| SEQ ID NO:6919 | 60/206,201 | 05/22/2000 |
| SEQ ID NO:6920 | 60/206,201 | 05/22/2000 |
| SEQ ID NO:6921 | 60/206,201 | 05/22/2000 |
| SEQ ID NO:6922 | 60/206,201 | 05/22/2000 |
| SEQ ID NO:6923 | 60/206,201 | 05/22/2000 |
| SEQ ID NO:6924 | 60/206,201 | 05/22/2000 |
| SEQ ID NO:6925 | 60/206,201 | 05/22/2000 |
| SEQ ID NO:6926 | 60/206,201 | 05/22/2000 |
| SEQ ID NO:6927 | 60/206,201 | 05/22/2000 |
| SEQ ID NO:6928 | 60/206,201 | 05/22/2000 |
| SEQ ID NO:6929 | 60/206,201 | 05/22/2000 |
| SEQ ID NO:6930 | 60/206,201 | 05/22/2000 |
| SEQ ID NO:6931 | 60/206,201 | 05/22/2000 |
| SEQ ID NO:6932 | 60/206,201 | 05/22/2000 |
| SEQ ID NO:6933 | 60/206,201 | 05/22/2000 |
| SEQ ID NO:6934 | 60/206,201 | 05/22/2000 |
| SEQ ID NO:6935 | 60/206,201 | 05/22/2000 |
| SEQ ID NO:6936 | 60/206,201 | 05/22/2000 |
| SEQ ID NO:6937 | 60/206,201 | 05/22/2000 |
| SEQ ID NO:6938 | 60/206,201 | 05/22/2000 |
| SEQ ID NO:6939 | 60/206,201 | 05/22/2000 |
| SEQ ID NO:6940 | 60/206,201 | 05/22/2000 |
| SEQ ID NO:6941 | 60/206,201 | 05/22/2000 |
| SEQ ID NO:6942 | 60/206,201 | 05/22/2000 |
| SEQ ID NO:6943 | 60/206,201 | 05/22/2000 |
| SEQ ID NO:6944 | 60/206,201 | 05/22/2000 |
| SEQ ID NO:6945 | 60/206,201 | 05/22/2000 |
| SEQ ID NO:6946 | 60/206,201 | 05/22/2000 |
| SEQ ID NO:6947 | 60/206,201 | 05/22/2000 |
| SEQ ID NO:6948 | 60/206,201 | 05/22/2000 |
| SEQ ID NO:6949 | 60/206,201 | 05/22/2000 |
| SEQ ID NO:6950 | 60/206,201 | 05/22/2000 |
| SEQ ID NO:6951 | 60/206,201 | 05/22/2000 |
| SEQ ID NO:6952 | 60/206,201 | 05/22/2000 |
| SEQ ID NO:6953 | 60/206,201 | 05/22/2000 |
| SEQ ID NO:6954 | 60/206,201 | 05/22/2000 |
| SEQ ID NO:6955 | 60/206,201 | 05/22/2000 |
| SEQ ID NO:6956 | 60/206,201 | 05/22/2000 |
| SEQ ID NO:6957 | 60/206,201 | 05/22/2000 |
| SEQ ID NO:6958 | 60/206,201 | 05/22/2000 |
| SEQ ID NO:6959 | 60/206,201 | 05/22/2000 |
| SEQ ID NO:6960 | 60/206,201 | 05/22/2000 |
| SEQ ID NO:6961 | 60/206,201 | 05/22/2000 |
| SEQ ID NO:6962 | 60/206,201 | 05/22/2000 |
| SEQ ID NO:6963 | 60/206,201 | 05/22/2000 |
| SEQ ID NO:6964 | 60/206,201 | 05/22/2000 |
| SEQ ID NO:6965 | 60/206,201 | 05/22/2000 |
| SEQ ID NO:6966 | 60/206,201 | 05/22/2000 |
| SEQ ID NO:6967 | 60/206,201 | 05/22/2000 |
| SEQ ID NO:6968 | 60/206,201 | 05/22/2000 |
| SEQ ID NO:6969 | 60/206,201 | 05/22/2000 |
| SEQ ID NO:6970 | 60/206,201 | 05/22/2000 |
| SEQ ID NO:6971 | 60/206,201 | 05/22/2000 |
| SEQ ID NO:6972 | 60/206,201 | 05/22/2000 |
| SEQ ID NO:6973 | 60/206,201 | 05/22/2000 |
| SEQ ID NO:6974 | 60/206,201 | 05/22/2000 |
| SEQ ID NO:6975 | 60/206,201 | 05/22/2000 |
| SEQ ID NO:6976 | 60/206,201 | 05/22/2000 |
| SEQ ID NO:6977 | 60/206,201 | 05/22/2000 |
| SEQ ID NO:6978 | 60/206,201 | 05/22/2000 |
| SEQ ID NO:6979 | 60/206,201 | 05/22/2000 |
| SEQ ID NO:6980 | 60/206,201 | 05/22/2000 |
| SEQ ID NO:6981 | 60/206,201 | 05/22/2000 |
| SEQ ID NO:6982 | 60/206,201 | 05/22/2000 |
| SEQ ID NO:6983 | 60/206,201 | 05/22/2000 |
| SEQ ID NO:6984 | 60/206,201 | 05/22/2000 |
| SEQ ID NO:6985 | 60/206,201 | 05/22/2000 |
| SEQ ID NO:6986 | 60/206,201 | 05/22/2000 |
| SEQ ID NO:6987 | 60/206,201 | 05/22/2000 |
| SEQ ID NO:6988 | 60/206,201 | 05/22/2000 |
| SEQ ID NO:6989 | 60/206,201 | 05/22/2000 |
| SEQ ID NO:6990 | 60/206,201 | 05/22/2000 |
| SEQ ID NO:6991 | 60/206,201 | 05/22/2000 |
| SEQ ID NO:6992 | 60/206,201 | 05/22/2000 |
| SEQ ID NO:6993 | 60/206,201 | 05/22/2000 |
| SEQ ID NO:6994 | 60/206,201 | 05/22/2000 |
| SEQ ID NO:6995 | 60/206,201 | 05/22/2000 |
| SEQ ID NO:6996 | 60/206,201 | 05/22/2000 |
| SEQ ID NO:6997 | 60/206,201 | 05/22/2000 |
| SEQ ID NO:6998 | 60/206,201 | 05/22/2000 |
| SEQ ID NO:6999 | 60/206,201 | 05/22/2000 |
| SEQ ID NO:7000 | 60/206,201 | 05/22/2000 |
| SEQ ID NO:7001 | 60/206,201 | 05/22/2000 |
| SEQ ID NO:7002 | 60/206,201 | 05/22/2000 |
| SEQ ID NO:7003 | 60/206,201 | 05/22/2000 |
| SEQ ID NO:7004 | 60/206,201 | 05/22/2000 |
| SEQ ID NO:7005 | 60/206,201 | 05/22/2000 |
| SEQ ID NO:7006 | 60/206,201 | 05/22/2000 |
| SEQ ID NO:7007 | 60/206,201 | 05/22/2000 |
| SEQ ID NO:7008 | 60/206,201 | 05/22/2000 |
| SEQ ID NO:7009 | 60/206,201 | 05/22/2000 |
| SEQ ID NO:7010 | 60/206,201 | 05/22/2000 |
| SEQ ID NO:7011 | 60/206,201 | 05/22/2000 |
| SEQ ID NO:7012 | 60/206,201 | 05/22/2000 |
| SEQ ID NO:7013 | 60/206,201 | 05/22/2000 |
| SEQ ID NO:7014 | 60/206,201 | 05/22/2000 |
| SEQ ID NO:7015 | 60/206,201 | 05/22/2000 |
| SEQ ID NO:7016 | 60/206,201 | 05/22/2000 |
| SEQ ID NO:7017 | 60/206,201 | 05/22/2000 |
| SEQ ID NO:7018 | 60/206,201 | 05/22/2000 |
| SEQ ID NO:7019 | 60/206,201 | 05/22/2000 |
| SEQ ID NO:7020 | 60/206,201 | 05/22/2000 |
| SEQ ID NO:7021 | 60/206,201 | 05/22/2000 |
| SEQ ID NO:7022 | 60/206,201 | 05/22/2000 |
| SEQ ID NO:7023 | 60/206,201 | 05/22/2000 |
| SEQ ID NO:7024 | 60/206,201 | 05/22/2000 |
| SEQ ID NO:7025 | 60/206,201 | 05/22/2000 |
| SEQ ID NO:7026 | 60/206,201 | 05/22/2000 |
| SEQ ID NO:7027 | 60/206,201 | 05/22/2000 |
| SEQ ID NO:7028 | 60/206,201 | 05/22/2000 |
| SEQ ID NO:7029 | 60/206,201 | 05/22/2000 |
| SEQ ID NO:7030 | 60/206,201 | 05/22/2000 |
| SEQ ID NO:7031 | 60/206,201 | 05/22/2000 |
| SEQ ID NO:7032 | 60/206,201 | 05/22/2000 |
| SEQ ID NO:7033 | 60/206,201 | 05/22/2000 |
| SEQ ID NO:7034 | 60/206,201 | 05/22/2000 |
| SEQ ID NO:7035 | 60/206,201 | 05/22/2000 |
| SEQ ID NO:7036 | 60/206,201 | 05/22/2000 |
| SEQ ID NO:7037 | 60/206,201 | 05/22/2000 |
| SEQ ID NO:7038 | 60/206,201 | 05/22/2000 |
| SEQ ID NO:7039 | 60/206,201 | 05/22/2000 |
| SEQ ID NO:7040 | 60/206,201 | 05/22/2000 |
| SEQ ID NO:7041 | 60/206,201 | 05/22/2000 |
| SEQ ID NO:7042 | 60/206,201 | 05/22/2000 |
| SEQ ID NO:7043 | 60/206,201 | 05/22/2000 |
| SEQ ID NO:7044 | 60/206,201 | 05/22/2000 |
| SEQ ID NO:7045 | 60/206,201 | 05/22/2000 |
| SEQ ID NO:7046 | 60/206,201 | 05/22/2000 |
| SEQ ID NO:7047 | 60/206,201 | 05/22/2000 |
| SEQ ID NO:7048 | 60/206,201 | 05/22/2000 |
| SEQ ID NO:7049 | 60/206,201 | 05/22/2000 |
| SEQ ID NO:7050 | 60/206,201 | 05/22/2000 |
| SEQ ID NO:7051 | 60/206,201 | 05/22/2000 |
| SEQ ID NO:7052 | 60/206,201 | 05/22/2000 |
| SEQ ID NO:7053 | 60/206,201 | 05/22/2000 |
| SEQ ID NO:7054 | 60/206,201 | 05/22/2000 |
| SEQ ID NO:7055 | 60/206,201 | 05/22/2000 |
| SEQ ID NO:7056 | 60/206,201 | 05/22/2000 |
| SEQ ID NO:7057 | 60/206,201 | 05/22/2000 |
| SEQ ID NO:7058 | 60/206,201 | 05/22/2000 |
| SEQ ID NO:7059 | 60/206,201 | 05/22/2000 |
| SEQ ID NO:7060 | 60/206,201 | 05/22/2000 |
| SEQ ID NO:7061 | 60/206,201 | 05/22/2000 |
| SEQ ID NO:7062 | 60/206,201 | 05/22/2000 |
| SEQ ID NO:7063 | 60/206,201 | 05/22/2000 |
| SEQ ID NO:7064 | 60/206,201 | 05/22/2000 |
| SEQ ID NO:7065 | 60/206,201 | 05/22/2000 |
| SEQ ID NO:7066 | 60/206,201 | 05/22/2000 |
| SEQ ID NO:7067 | 60/206,201 | 05/22/2000 |
| SEQ ID NO:7068 | 60/206,201 | 05/22/2000 |
| SEQ ID NO:7069 | 60/206,201 | 05/22/2000 |
| SEQ ID NO:7070 | 60/206,201 | 05/22/2000 |
| SEQ ID NO:7071 | 60/206,201 | 05/22/2000 |
| SEQ ID NO:7072 | 60/206,201 | 05/22/2000 |
| SEQ ID NO:7073 | 60/206,201 | 05/22/2000 |
| SEQ ID NO:7074 | 60/206,201 | 05/22/2000 |
| SEQ ID NO:7075 | 60/206,201 | 05/22/2000 |
| SEQ ID NO:7076 | 60/206,201 | 05/22/2000 |
| SEQ ID NO:7077 | 60/206,201 | 05/22/2000 |
| SEQ ID NO:7078 | 60/206,201 | 05/22/2000 |
| SEQ ID NO:7079 | 60/206,201 | 05/22/2000 |
| SEQ ID NO:7080 | 60/206,201 | 05/22/2000 |
| SEQ ID NO:7081 | 60/206,201 | 05/22/2000 |
| SEQ ID NO:7082 | 60/206,201 | 05/22/2000 |
| SEQ ID NO:7083 | 60/206,201 | 05/22/2000 |
| SEQ ID NO:7084 | 60/206,201 | 05/22/2000 |
| SEQ ID NO:7085 | 60/206,201 | 05/22/2000 |
| SEQ ID NO:7086 | 60/206,201 | 05/22/2000 |
| SEQ ID NO:7087 | 60/206,201 | 05/22/2000 |
| SEQ ID NO:7088 | 60/206,201 | 05/22/2000 |
| SEQ ID NO:7089 | 60/206,201 | 05/22/2000 |
| SEQ ID NO:7090 | 60/206,201 | 05/22/2000 |
| SEQ ID NO:7091 | 60/206,201 | 05/22/2000 |
| SEQ ID NO:7092 | 60/206,201 | 05/22/2000 |
| SEQ ID NO:7093 | 60/206,201 | 05/22/2000 |
| SEQ ID NO:7094 | 60/206,201 | 05/22/2000 |
| SEQ ID NO:7095 | 60/206,201 | 05/22/2000 |
| SEQ ID NO:7096 | 60/206,201 | 05/22/2000 |
| SEQ ID NO:7097 | 60/206,201 | 05/22/2000 |
| SEQ ID NO:7098 | 60/206,201 | 05/22/2000 |
| SEQ ID NO:7099 | 60/206,201 | 05/22/2000 |
| SEQ ID NO:7100 | 60/206,201 | 05/22/2000 |
| SEQ ID NO:7101 | 60/206,201 | 05/22/2000 |
| SEQ ID NO:7102 | 60/206,201 | 05/22/2000 |
| SEQ ID NO:7103 | 60/206,201 | 05/22/2000 |
| SEQ ID NO:7104 | 60/206,201 | 05/22/2000 |
| SEQ ID NO:7105 | 60/206,201 | 05/22/2000 |
| SEQ ID NO:7106 | 60/206,201 | 05/22/2000 |
| SEQ ID NO:7107 | 60/206,201 | 05/22/2000 |
| SEQ ID NO:7108 | 60/206,201 | 05/22/2000 |
| SEQ ID NO:7109 | 60/206,201 | 05/22/2000 |
| SEQ ID NO:7110 | 60/206,201 | 05/22/2000 |
| SEQ ID NO:7111 | 60/206,201 | 05/22/2000 |
| SEQ ID NO:7112 | 60/206,201 | 05/22/2000 |
| SEQ ID NO:7113 | 60/206,201 | 05/22/2000 |
| SEQ ID NO:7114 | 60/206,201 | 05/22/2000 |
| SEQ ID NO:7115 | 60/206,201 | 05/22/2000 |
| SEQ ID NO:7116 | 60/206,201 | 05/22/2000 |
| SEQ ID NO:7117 | 60/206,201 | 05/22/2000 |
| SEQ ID NO:7118 | 60/206,201 | 05/22/2000 |
| SEQ ID NO:7119 | 60/206,201 | 05/22/2000 |
| SEQ ID NO:7120 | 60/206,201 | 05/22/2000 |
| SEQ ID NO:7121 | 60/206,201 | 05/22/2000 |
| SEQ ID NO:7122 | 60/206,201 | 05/22/2000 |
| SEQ ID NO:7123 | 60/206,201 | 05/22/2000 |
| SEQ ID NO:7124 | 60/206,201 | 05/22/2000 |
| SEQ ID NO:7125 | 60/206,201 | 05/22/2000 |
| SEQ ID NO:7126 | 60/206,201 | 05/22/2000 |
| SEQ ID NO:7127 | 60/206,201 | 05/22/2000 |
| SEQ ID NO:7128 | 60/206,201 | 05/22/2000 |
| SEQ ID NO:7129 | 60/206,201 | 05/22/2000 |
| SEQ ID NO:7130 | 60/206,201 | 05/22/2000 |
| SEQ ID NO:7131 | 60/206,201 | 05/22/2000 |
| SEQ ID NO:7132 | 60/206,201 | 05/22/2000 |
| SEQ ID NO:7133 | 60/206,201 | 05/22/2000 |
| SEQ ID NO:7134 | 60/206,201 | 05/22/2000 |
| SEQ ID NO:7135 | 60/206,201 | 05/22/2000 |
| SEQ ID NO:7136 | 60/206,201 | 05/22/2000 |
| SEQ ID NO:7137 | 60/206,201 | 05/22/2000 |
| SEQ ID NO:7138 | 60/206,201 | 05/22/2000 |
| SEQ ID NO:7139 | 60/206,201 | 05/22/2000 |
| SEQ ID NO:7140 | 60/206,201 | 05/22/2000 |
| SEQ ID NO:7141 | 60/206,201 | 05/22/2000 |
| SEQ ID NO:7142 | 60/206,201 | 05/22/2000 |
| SEQ ID NO:7143 | 60/206,201 | 05/22/2000 |
| SEQ ID NO:7144 | 60/206,201 | 05/22/2000 |
| SEQ ID NO:7145 | 60/206,201 | 05/22/2000 |
| SEQ ID NO:7146 | 60/206,201 | 05/22/2000 |
| SEQ ID NO:7147 | 60/206,201 | 05/22/2000 |
| SEQ ID NO:7148 | 60/206,201 | 05/22/2000 |
| SEQ ID NO:7149 | 60/206,201 | 05/22/2000 |
| SEQ ID NO:7150 | 60/206,201 | 05/22/2000 |
| SEQ ID NO:7151 | 60/206,201 | 05/22/2000 |
| SEQ ID NO:7152 | 60/206,201 | 05/22/2000 |
| SEQ ID NO:7153 | 60/206,201 | 05/22/2000 |
| SEQ ID NO:7154 | 60/206,201 | 05/22/2000 |
| SEQ ID NO:7155 | 60/206,201 | 05/22/2000 |
| SEQ ID NO:7156 | 60/206,201 | 05/22/2000 |
| SEQ ID NO:7157 | 60/206,201 | 05/22/2000 |
| SEQ ID NO:7158 | 60/206,201 | 05/22/2000 |
| SEQ ID NO:7159 | 60/206,201 | 05/22/2000 |
| SEQ ID NO:7160 | 60/206,201 | 05/22/2000 |
| SEQ ID NO:7161 | 60/206,201 | 05/22/2000 |
| SEQ ID NO:7162 | 60/206,201 | 05/22/2000 |
| SEQ ID NO:7163 | 60/206,201 | 05/22/2000 |
| SEQ ID NO:7164 | 60/206,201 | 05/22/2000 |
| SEQ ID NO:7165 | 60/206,201 | 05/22/2000 |
| SEQ ID NO:7166 | 60/206,201 | 05/22/2000 |
| SEQ ID NO:7167 | 60/206,201 | 05/22/2000 |
| SEQ ID NO:7168 | 60/206,201 | 05/22/2000 |
| SEQ ID NO:7169 | 60/206,201 | 05/22/2000 |
| SEQ ID NO:7170 | 60/206,201 | 05/22/2000 |
| SEQ ID NO:7171 | 60/206,201 | 05/22/2000 |
| SEQ ID NO:7172 | 60/206,201 | 05/22/2000 |
| SEQ ID NO:7173 | 60/206,201 | 05/22/2000 |
| SEQ ID NO:7174 | 60/206,201 | 05/22/2000 |
| SEQ ID NO:7175 | 60/206,201 | 05/22/2000 |
| SEQ ID NO:7176 | 60/206,201 | 05/22/2000 |
| SEQ ID NO:7177 | 60/206,201 | 05/22/2000 |
| SEQ ID NO:7178 | 60/206,201 | 05/22/2000 |
| SEQ ID NO:7179 | 60/206,201 | 05/22/2000 |
| SEQ ID NO:7180 | 60/206,201 | 05/22/2000 |
| SEQ ID NO:7181 | 60/206,201 | 05/22/2000 |
| SEQ ID NO:7182 | 60/206,201 | 05/22/2000 |
| SEQ ID NO:7183 | 60/206,201 | 05/22/2000 |
| SEQ ID NO:7184 | 60/206,201 | 05/22/2000 |
| SEQ ID NO:7185 | 60/206,201 | 05/22/2000 |
| SEQ ID NO:7186 | 60/206,201 | 05/22/2000 |
| SEQ ID NO:7187 | 60/206,201 | 05/22/2000 |
| SEQ ID NO:7188 | 60/206,201 | 05/22/2000 |
| SEQ ID NO:7189 | 60/206,201 | 05/22/2000 |
| SEQ ID NO:7190 | 60/206,201 | 05/22/2000 |
| SEQ ID NO:7191 | 60/206,201 | 05/22/2000 |
| SEQ ID NO:7192 | 60/206,201 | 05/22/2000 |
| SEQ ID NO:7193 | 60/206,201 | 05/22/2000 |
| SEQ ID NO:7194 | 60/206,201 | 05/22/2000 |
| SEQ ID NO:7195 | 60/206,201 | 05/22/2000 |
| SEQ ID NO:7196 | 60/206,201 | 05/22/2000 |
| SEQ ID NO:7197 | 60/206,201 | 05/22/2000 |
| SEQ ID NO:7198 | 60/206,201 | 05/22/2000 |
| SEQ ID NO:7199 | 60/206,201 | 05/22/2000 |
| SEQ ID NO:7200 | 60/206,201 | 05/22/2000 |
| SEQ ID NO:7201 | 60/206,201 | 05/22/2000 |
| SEQ ID NO:7202 | 60/206,201 | 05/22/2000 |
| SEQ ID NO:7203 | 60/206,201 | 05/22/2000 |
| SEQ ID NO:7204 | 60/206,201 | 05/22/2000 |
| SEQ ID NO:7205 | 60/206,201 | 05/22/2000 |
| SEQ ID NO:7206 | 60/206,201 | 05/22/2000 |
| SEQ ID NO:7207 | 60/206,201 | 05/22/2000 |
| SEQ ID NO:7208 | 60/206,201 | 05/22/2000 |
| SEQ ID NO:7209 | 60/206,201 | 05/22/2000 |
| SEQ ID NO:7210 | 60/206,201 | 05/22/2000 |
| SEQ ID NO:7211 | 60/206,201 | 05/22/2000 |
| SEQ ID NO:7212 | 60/206,201 | 05/22/2000 |
| SEQ ID NO:7213 | 60/206,201 | 05/22/2000 |
| SEQ ID NO:7214 | 60/206,201 | 05/22/2000 |
| SEQ ID NO:7215 | 60/206,201 | 05/22/2000 |
| SEQ ID NO:7216 | 60/206,201 | 05/22/2000 |
| SEQ ID NO:7217 | 60/206,201 | 05/22/2000 |
| SEQ ID NO:7218 | 60/206,201 | 05/22/2000 |
| SEQ ID NO:7219 | 60/206,201 | 05/22/2000 |
| SEQ ID NO:7220 | 60/206,201 | 05/22/2000 |
| SEQ ID NO:7221 | 60/206,201 | 05/22/2000 |
| SEQ ID NO:7222 | 60/206,201 | 05/22/2000 |
| SEQ ID NO:7223 | 60/206,201 | 05/22/2000 |
| SEQ ID NO:7224 | 60/206,201 | 05/22/2000 |
| SEQ ID NO:7225 | 60/206,201 | 05/22/2000 |
| SEQ ID NO:7226 | 60/206,201 | 05/22/2000 |
| SEQ ID NO:7227 | 60/206,201 | 05/22/2000 |
| SEQ ID NO:7228 | 60/206,201 | 05/22/2000 |
| SEQ ID NO:7229 | 60/206,201 | 05/22/2000 |
| SEQ ID NO:7230 | 60/206,201 | 05/22/2000 |
| SEQ ID NO:7231 | 60/206,201 | 05/22/2000 |
| SEQ ID NO:7232 | 60/206,201 | 05/22/2000 |
| SEQ ID NO:7233 | 60/206,201 | 05/22/2000 |
| SEQ ID NO:7234 | 60/206,201 | 05/22/2000 |
| SEQ ID NO:7235 | 60/206,201 | 05/22/2000 |
| SEQ ID NO:7236 | 60/206,201 | 05/22/2000 |
| SEQ ID NO:7237 | 60/206,201 | 05/22/2000 |
| SEQ ID NO:7238 | 60/206,201 | 05/22/2000 |
| SEQ ID NO:7239 | 60/206,201 | 05/22/2000 |
| SEQ ID NO:7240 | 60/206,201 | 05/22/2000 |
| SEQ ID NO:7241 | 60/206,201 | 05/22/2000 |
| SEQ ID NO:7242 | 60/206,201 | 05/22/2000 |
| SEQ ID NO:7243 | 60/206,201 | 05/22/2000 |
| SEQ ID NO:7244 | 60/206,201 | 05/22/2000 |
| SEQ ID NO:7245 | 60/206,201 | 05/22/2000 |
| SEQ ID NO:7246 | 60/206,201 | 05/22/2000 |
| SEQ ID NO:7247 | 60/206,201 | 05/22/2000 |
| SEQ ID NO:7248 | 60/206,201 | 05/22/2000 |
| SEQ ID NO:7249 | 60/206,201 | 05/22/2000 |
| SEQ ID NO:7250 | 60/206,201 | 05/22/2000 |
| SEQ ID NO:7251 | 60/206,201 | 05/22/2000 |
| SEQ ID NO:7252 | 60/206,201 | 05/22/2000 |
| SEQ ID NO:7253 | 60/206,201 | 05/22/2000 |
| SEQ ID NO:7254 | 60/206,201 | 05/22/2000 |
| SEQ ID NO:7255 | 60/206,201 | 05/22/2000 |
| SEQ ID NO:7256 | 60/206,201 | 05/22/2000 |
| SEQ ID NO:7257 | 60/206,201 | 05/22/2000 |
| SEQ ID NO:7258 | 60/206,201 | 05/22/2000 |
| SEQ ID NO:7259 | 60/206,201 | 05/22/2000 |
| SEQ ID NO:7260 | 60/206,201 | 05/22/2000 |
| SEQ ID NO:7261 | 60/206,201 | 05/22/2000 |
| SEQ ID NO:7262 | 60/206,201 | 05/22/2000 |
| SEQ ID NO:7263 | 60/206,201 | 05/22/2000 |
| SEQ ID NO:7264 | 60/206,201 | 05/22/2000 |
| SEQ ID NO:7265 | 60/206,201 | 05/22/2000 |
| SEQ ID NO:7266 | 60/206,201 | 05/22/2000 |
| SEQ ID NO:7267 | 60/206,201 | 05/22/2000 |
| SEQ ID NO:7268 | 60/206,201 | 05/22/2000 |
| SEQ ID NO:7269 | 60/206,201 | 05/22/2000 |
| SEQ ID NO:7270 | 60/206,201 | 05/22/2000 |
| SEQ ID NO:7271 | 60/206,201 | 05/22/2000 |
| SEQ ID NO:7272 | 60/206,201 | 05/22/2000 |
| SEQ ID NO:7273 | 60/206,201 | 05/22/2000 |
| SEQ ID NO:7274 | 60/206,201 | 05/22/2000 |
| SEQ ID NO:7275 | 60/206,201 | 05/22/2000 |
| SEQ ID NO:7276 | 60/206,201 | 05/22/2000 |
| SEQ ID NO:7277 | 60/206,201 | 05/22/2000 |
| SEQ ID NO:7278 | 60/206,201 | 05/22/2000 |
| SEQ ID NO:7279 | 60/206,201 | 05/22/2000 |
| SEQ ID NO:7280 | 60/206,201 | 05/22/2000 |
| SEQ ID NO:7281 | 60/206,201 | 05/22/2000 |
| SEQ ID NO:7282 | 60/206,201 | 05/22/2000 |
| SEQ ID NO:7283 | 60/206,201 | 05/22/2000 |
| SEQ ID NO:7284 | 60/206,201 | 05/22/2000 |
| SEQ ID NO:7285 | 60/206,201 | 05/22/2000 |
| SEQ ID NO:7286 | 60/206,201 | 05/22/2000 |
| SEQ ID NO:7287 | 60/206,201 | 05/22/2000 |
| SEQ ID NO:7288 | 60/206,201 | 05/22/2000 |
| SEQ ID NO:7289 | 60/206,201 | 05/22/2000 |
| SEQ ID NO:7290 | 60/206,201 | 05/22/2000 |
| SEQ ID NO:7291 | 60/206,201 | 05/22/2000 |
| SEQ ID NO:7292 | 60/206,201 | 05/22/2000 |
| SEQ ID NO:7293 | 60/206,201 | 05/22/2000 |
| SEQ ID NO:7294 | 60/206,201 | 05/22/2000 |
| SEQ ID NO:7295 | 60/206,201 | 05/22/2000 |
| SEQ ID NO:7296 | 60/206,201 | 05/22/2000 |
| SEQ ID NO:7297 | 60/206,201 | 05/22/2000 |
| SEQ ID NO:7298 | 60/206,201 | 05/22/2000 |
| SEQ ID NO:7299 | 60/206,201 | 05/22/2000 |
| SEQ ID NO:7300 | 60/206,201 | 05/22/2000 |
| SEQ ID NO:7301 | 60/206,201 | 05/22/2000 |
| SEQ ID NO:7302 | 60/206,201 | 05/22/2000 |
| SEQ ID NO:7303 | 60/206,201 | 05/22/2000 |
| SEQ ID NO:7304 | 60/206,201 | 05/22/2000 |
| SEQ ID NO:7305 | 60/206,201 | 05/22/2000 |
| SEQ ID NO:7306 | 60/206,201 | 05/22/2000 |
| SEQ ID NO:7307 | 60/206,201 | 05/22/2000 |
| SEQ ID NO:7308 | 60/206,201 | 05/22/2000 |
| SEQ ID NO:7309 | 60/206,201 | 05/22/2000 |
| SEQ ID NO:7310 | 60/206,201 | 05/22/2000 |
| SEQ ID NO:7311 | 60/206,201 | 05/22/2000 |
| SEQ ID NO:7312 | 60/206,201 | 05/22/2000 |
| SEQ ID NO:7313 | 60/206,201 | 05/22/2000 |
| SEQ ID NO:7314 | 60/206,201 | 05/22/2000 |
| SEQ ID NO:7315 | 60/206,201 | 05/22/2000 |
| SEQ ID NO:7316 | 60/206,201 | 05/22/2000 |
| SEQ ID NO:7317 | 60/206,201 | 05/22/2000 |
| SEQ ID NO:7318 | 60/206,201 | 05/22/2000 |
| SEQ ID NO:7319 | 60/206,201 | 05/22/2000 |
| SEQ ID NO:7320 | 60/206,201 | 05/22/2000 |
| SEQ ID NO:7321 | 60/206,201 | 05/22/2000 |
| SEQ ID NO:7322 | 60/206,201 | 05/22/2000 |
| SEQ ID NO:7323 | 60/206,201 | 05/22/2000 |
| SEQ ID NO:7324 | 60/206,201 | 05/22/2000 |
| SEQ ID NO:7325 | 60/206,201 | 05/22/2000 |
| SEQ ID NO:7326 | 60/206,201 | 05/22/2000 |
| SEQ ID NO:7327 | 60/206,201 | 05/22/2000 |
| SEQ ID NO:7328 | 60/206,201 | 05/22/2000 |
| SEQ ID NO:7329 | 60/206,201 | 05/22/2000 |
| SEQ ID NO:7330 | 60/206,201 | 05/22/2000 |
| SEQ ID NO:7331 | 60/206,201 | 05/22/2000 |
| SEQ ID NO:7332 | 60/206,201 | 05/22/2000 |
| SEQ ID NO:7333 | 60/206,201 | 05/22/2000 |
| SEQ ID NO:7334 | 60/206,201 | 05/22/2000 |
| SEQ ID NO:7335 | 60/206,201 | 05/22/2000 |
| SEQ ID NO:7336 | 60/206,201 | 05/22/2000 |
| SEQ ID NO:7337 | 60/206,201 | 05/22/2000 |
| SEQ ID NO:7338 | 60/206,201 | 05/22/2000 |
| SEQ ID NO:7339 | 60/206,201 | 05/22/2000 |
| SEQ ID NO:7340 | 60/206,201 | 05/22/2000 |
| SEQ ID NO:7341 | 60/206,201 | 05/22/2000 |
| SEQ ID NO:7342 | 60/206,201 | 05/22/2000 |
| SEQ ID NO:7343 | 60/206,201 | 05/22/2000 |
| SEQ ID NO:7344 | 60/206,201 | 05/22/2000 |
| SEQ ID NO:7345 | 60/206,201 | 05/22/2000 |
| SEQ ID NO:7346 | 60/206,201 | 05/22/2000 |
| SEQ ID NO:7347 | 60/206,201 | 05/22/2000 |
| SEQ ID NO:7348 | 60/206,201 | 05/22/2000 |
| SEQ ID NO:7349 | 60/206,201 | 05/22/2000 |
| SEQ ID NO:7350 | 60/206,201 | 05/22/2000 |
| SEQ ID NO:7351 | 60/206,201 | 05/22/2000 |
| SEQ ID NO:7352 | 60/206,201 | 05/22/2000 |
| SEQ ID NO:7353 | 60/206,201 | 05/22/2000 |
| SEQ ID NO:7354 | 60/206,201 | 05/22/2000 |
| SEQ ID NO:7355 | 60/206,201 | 05/22/2000 |
| SEQ ID NO:7356 | 60/206,201 | 05/22/2000 |
| SEQ ID NO:7357 | 60/206,201 | 05/22/2000 |
| SEQ ID NO:7358 | 60/206,201 | 05/22/2000 |
| SEQ ID NO:7359 | 60/206,201 | 05/22/2000 |
| SEQ ID NO:7360 | 60/206,201 | 05/22/2000 |
| SEQ ID NO:7361 | 60/206,201 | 05/22/2000 |
| SEQ ID NO:7362 | 60/206,201 | 05/22/2000 |
| SEQ ID NO:7363 | 60/206,201 | 05/22/2000 |
| SEQ ID NO:7364 | 60/206,201 | 05/22/2000 |
| SEQ ID NO:7365 | 60/206,201 | 05/22/2000 |
| SEQ ID NO:7366 | 60/206,201 | 05/22/2000 |
| SEQ ID NO:7367 | 60/206,201 | 05/22/2000 |
| SEQ ID NO:7368 | 60/206,201 | 05/22/2000 |
| SEQ ID NO:7369 | 60/206,201 | 05/22/2000 |
| SEQ ID NO:7370 | 60/206,201 | 05/22/2000 |
| SEQ ID NO:7371 | 60/206,201 | 05/22/2000 |
| SEQ ID NO:7372 | 60/206,201 | 05/22/2000 |
| SEQ ID NO:7373 | 60/206,201 | 05/22/2000 |
| SEQ ID NO:7374 | 60/206,201 | 05/22/2000 |
| SEQ ID NO:7375 | 60/206,201 | 05/22/2000 |
| SEQ ID NO:7376 | 60/206,201 | 05/22/2000 |
| SEQ ID NO:7377 | 60/206,201 | 05/22/2000 |
| SEQ ID NO:7378 | 60/206,201 | 05/22/2000 |
| SEQ ID NO:7379 | 60/206,201 | 05/22/2000 |
| SEQ ID NO:7380 | 60/206,201 | 05/22/2000 |
| SEQ ID NO:7381 | 60/206,201 | 05/22/2000 |
| SEQ ID NO:7382 | 60/206,201 | 05/22/2000 |
| SEQ ID NO:7383 | 60/206,201 | 05/22/2000 |
| SEQ ID NO:7384 | 60/206,201 | 05/22/2000 |
| SEQ ID NO:7385 | 60/206,201 | 05/22/2000 |
| SEQ ID NO:7386 | 60/206,201 | 05/22/2000 |
| SEQ ID NO:7387 | 60/206,201 | 05/22/2000 |
| SEQ ID NO:7388 | 60/206,201 | 05/22/2000 |
| SEQ ID NO:7389 | 60/206,201 | 05/22/2000 |
| SEQ ID NO:7390 | 60/206,201 | 05/22/2000 |
| SEQ ID NO:7391 | 60/206,201 | 05/22/2000 |
| SEQ ID NO:7392 | 60/206,201 | 05/22/2000 |
| SEQ ID NO:7393 | 60/206,201 | 05/22/2000 |
| SEQ ID NO:7394 | 60/206,201 | 05/22/2000 |
| SEQ ID NO:7395 | 60/206,201 | 05/22/2000 |
| SEQ ID NO:7396 | 60/206,201 | 05/22/2000 |
| SEQ ID NO:7397 | 60/206,201 | 05/22/2000 |
| SEQ ID NO:7398 | 60/206,201 | 05/22/2000 |
| SEQ ID NO:7399 | 60/206,201 | 05/22/2000 |
| SEQ ID NO:7400 | 60/206,201 | 05/22/2000 |
| SEQ ID NO:7401 | 60/206,201 | 05/22/2000 |
| SEQ ID NO:7402 | 60/206,201 | 05/22/2000 |
| SEQ ID NO:7403 | 60/206,201 | 05/22/2000 |
| SEQ ID NO:7404 | 60/206,201 | 05/22/2000 |
| SEQ ID NO:7405 | 60/206,201 | 05/22/2000 |
| SEQ ID NO:7406 | 60/206,201 | 05/22/2000 |
| SEQ ID NO:7407 | 60/206,201 | 05/22/2000 |
| SEQ ID NO:7408 | 60/206,201 | 05/22/2000 |
| SEQ ID NO:7409 | 60/206,201 | 05/22/2000 |
| SEQ ID NO:7410 | 60/206,201 | 05/22/2000 |
| SEQ ID NO:7411 | 60/206,201 | 05/22/2000 |
| SEQ ID NO:7412 | 60/206,201 | 05/22/2000 |
| SEQ ID NO:7413 | 60/206,201 | 05/22/2000 |
| SEQ ID NO:7414 | 60/206,201 | 05/22/2000 |
| SEQ ID NO:7415 | 60/206,201 | 05/22/2000 |
| SEQ ID NO:7416 | 60/206,201 | 05/22/2000 |
| SEQ ID NO:7417 | 60/206,201 | 05/22/2000 |
| SEQ ID NO:7418 | 60/206,201 | 05/22/2000 |
| SEQ ID NO:7419 | 60/206,201 | 05/22/2000 |
| SEQ ID NO:7420 | 60/206,201 | 05/22/2000 |
| SEQ ID NO:7421 | 60/206,201 | 05/22/2000 |
| SEQ ID NO:7422 | 60/206,201 | 05/22/2000 |
| SEQ ID NO:7423 | 60/206,201 | 05/22/2000 |
| SEQ ID NO:7424 | 60/206,201 | 05/22/2000 |
| SEQ ID NO:7425 | 60/206,201 | 05/22/2000 |
| SEQ ID NO:7426 | 60/206,201 | 05/22/2000 |
| SEQ ID NO:7427 | 60/206,201 | 05/22/2000 |
| SEQ ID NO:7428 | 60/206,201 | 05/22/2000 |
| SEQ ID NO:7429 | 60/206,201 | 05/22/2000 |
| SEQ ID NO:7430 | 60/206,201 | 05/22/2000 |
| SEQ ID NO:7431 | 60/206,201 | 05/22/2000 |
| SEQ ID NO:7432 | 60/206,201 | 05/22/2000 |
| SEQ ID NO:7433 | 60/206,201 | 05/22/2000 |
| SEQ ID NO:7434 | 60/206,201 | 05/22/2000 |
| SEQ ID NO:7435 | 60/206,201 | 05/22/2000 |
| SEQ ID NO:7436 | 60/206,201 | 05/22/2000 |
| SEQ ID NO:7437 | 60/206,201 | 05/22/2000 |
| SEQ ID NO:7438 | 60/206,201 | 05/22/2000 |
| SEQ ID NO:7439 | 60/206,201 | 05/22/2000 |
| SEQ ID NO:7440 | 60/206,201 | 05/22/2000 |
| SEQ ID NO:7441 | 60/206,201 | 05/22/2000 |
| SEQ ID NO:7442 | 60/206,201 | 05/22/2000 |
| SEQ ID NO:7443 | 60/206,201 | 05/22/2000 |
| SEQ ID NO:7444 | 60/206,201 | 05/22/2000 |
| SEQ ID NO:7445 | 60/206,201 | 05/22/2000 |
| SEQ ID NO:7446 | 60/206,201 | 05/22/2000 |
| SEQ ID NO:7447 | 60/206,201 | 05/22/2000 |
| SEQ ID NO:7448 | 60/206,201 | 05/22/2000 |
| SEQ ID NO:7449 | 60/206,201 | 05/22/2000 |
| SEQ ID NO:7450 | 60/206,201 | 05/22/2000 |
| SEQ ID NO:7451 | 60/206,201 | 05/22/2000 |
| SEQ ID NO:7452 | 60/206,201 | 05/22/2000 |
| SEQ ID NO:7453 | 60/206,201 | 05/22/2000 |
| SEQ ID NO:7454 | 60/206,201 | 05/22/2000 |
| SEQ ID NO:7455 | 60/206,201 | 05/22/2000 |
| SEQ ID NO:7456 | 60/206,201 | 05/22/2000 |
| SEQ ID NO:7457 | 60/206,201 | 05/22/2000 |
| SEQ ID NO:7458 | 60/206,201 | 05/22/2000 |
| SEQ ID NO:7459 | 60/206,201 | 05/22/2000 |
| SEQ ID NO:7460 | 60/206,201 | 05/22/2000 |
| SEQ ID NO:7461 | 60/206,201 | 05/22/2000 |
| SEQ ID NO:7462 | 60/206,201 | 05/22/2000 |
| SEQ ID NO:7463 | 60/206,201 | 05/22/2000 |
| SEQ ID NO:7464 | 60/206,201 | 05/22/2000 |
| SEQ ID NO:7465 | 60/206,201 | 05/22/2000 |
| SEQ ID NO:7466 | 60/206,201 | 05/22/2000 |
| SEQ ID NO:7467 | 60/206,201 | 05/22/2000 |
| SEQ ID NO:7468 | 60/206,201 | 05/22/2000 |
| SEQ ID NO:7469 | 60/206,201 | 05/22/2000 |
| SEQ ID NO:7470 | 60/206,201 | 05/22/2000 |
| SEQ ID NO:7471 | 60/206,201 | 05/22/2000 |
| SEQ ID NO:7472 | 60/206,201 | 05/22/2000 |
| SEQ ID NO:7473 | 60/206,201 | 05/22/2000 |
| SEQ ID NO:7474 | 60/206,201 | 05/22/2000 |
| SEQ ID NO:7475 | 60/206,201 | 05/22/2000 |
| SEQ ID NO:7476 | 60/206,201 | 05/22/2000 |
| SEQ ID NO:7477 | 60/206,201 | 05/22/2000 |
| SEQ ID NO:7478 | 60/206,201 | 05/22/2000 |
| SEQ ID NO:7479 | 60/206,201 | 05/22/2000 |
| SEQ ID NO:7480 | 60/206,201 | 05/22/2000 |
| SEQ ID NO:7481 | 60/206,201 | 05/22/2000 |
| SEQ ID NO:7482 | 60/206,201 | 05/22/2000 |
| SEQ ID NO:7483 | 60/206,201 | 05/22/2000 |
| SEQ ID NO:7484 | 60/206,201 | 05/22/2000 |
| SEQ ID NO:7485 | 60/206,201 | 05/22/2000 |
| SEQ ID NO:7486 | 60/206,201 | 05/22/2000 |
| SEQ ID NO:7487 | 60/206,201 | 05/22/2000 |
| SEQ ID NO:7488 | 60/206,201 | 05/22/2000 |
| SEQ ID NO:7489 | 60/206,201 | 05/22/2000 |
| SEQ ID NO:7490 | 60/206,201 | 05/22/2000 |
| SEQ ID NO:7491 | 60/206,201 | 05/22/2000 |
| SEQ ID NO:7492 | 60/206,201 | 05/22/2000 |
| SEQ ID NO:7493 | 60/206,201 | 05/22/2000 |
| SEQ ID NO:7494 | 60/206,201 | 05/22/2000 |
| SEQ ID NO:7495 | 60/206,201 | 05/22/2000 |
| SEQ ID NO:7496 | 60/206,201 | 05/22/2000 |
| SEQ ID NO:7497 | 60/206,201 | 05/22/2000 |
| SEQ ID NO:7498 | 60/206,201 | 05/22/2000 |
| SEQ ID NO:7499 | 60/206,201 | 05/22/2000 |
| SEQ ID NO:7500 | 60/206,201 | 05/22/2000 |
| SEQ ID NO:7501 | 60/206,201 | 05/22/2000 |
| SEQ ID NO:7502 | 60/206,201 | 05/22/2000 |
| SEQ ID NO:7503 | 60/206,201 | 05/22/2000 |
| SEQ ID NO:7504 | 60/206,201 | 05/22/2000 |
| SEQ ID NO:7505 | 60/206,201 | 05/22/2000 |
| SEQ ID NO:7506 | 60/206,201 | 05/22/2000 |
| SEQ ID NO:7507 | 60/206,201 | 05/22/2000 |
| SEQ ID NO:7508 | 60/206,201 | 05/22/2000 |
| SEQ ID NO:7509 | 60/206,201 | 05/22/2000 |
| SEQ ID NO:7510 | 60/206,201 | 05/22/2000 |
| SEQ ID NO:7511 | 60/206,201 | 05/22/2000 |
| SEQ ID NO:7512 | 60/206,201 | 05/22/2000 |
| SEQ ID NO:7513 | 60/206,201 | 05/22/2000 |
| SEQ ID NO:7514 | 60/206,201 | 05/22/2000 |
| SEQ ID NO:7515 | 60/206,201 | 05/22/2000 |
| SEQ ID NO:7516 | 60/206,201 | 05/22/2000 |
| SEQ ID NO:7517 | 60/206,201 | 05/22/2000 |
| SEQ ID NO:7518 | 60/206,201 | 05/22/2000 |
| SEQ ID NO:7519 | 60/206,201 | 05/22/2000 |
| SEQ ID NO:7520 | 60/206,201 | 05/22/2000 |
| SEQ ID NO:7521 | 60/206,201 | 05/22/2000 |
| SEQ ID NO:7522 | 60/206,201 | 05/22/2000 |
| SEQ ID NO:7523 | 60/206,201 | 05/22/2000 |
| SEQ ID NO:7524 | 60/206,201 | 05/22/2000 |
| SEQ ID NO:7525 | 60/206,201 | 05/22/2000 |
| SEQ ID NO:7526 | 60/206,201 | 05/22/2000 |
| SEQ ID NO:7527 | 60/206,201 | 05/22/2000 |
| SEQ ID NO:7528 | 60/206,201 | 05/22/2000 |
| SEQ ID NO:7529 | 60/206,201 | 05/22/2000 |
| SEQ ID NO:7530 | 60/206,201 | 05/22/2000 |
| SEQ ID NO:7531 | 60/206,201 | 05/22/2000 |
| SEQ ID NO:7532 | 60/206,201 | 05/22/2000 |
| SEQ ID NO:7533 | 60/206,201 | 05/22/2000 |
| SEQ ID NO:7534 | 60/206,201 | 05/22/2000 |
| SEQ ID NO:7535 | 60/206,201 | 05/22/2000 |
| SEQ ID NO:7536 | 60/206,201 | 05/22/2000 |
| SEQ ID NO:7537 | 60/206,201 | 05/22/2000 |
| SEQ ID NO:7538 | 60/206,201 | 05/22/2000 |
| SEQ ID NO:7539 | 60/206,201 | 05/22/2000 |
| SEQ ID NO:7540 | 60/206,201 | 05/22/2000 |
| SEQ ID NO:7541 | 60/206,201 | 05/22/2000 |
| SEQ ID NO:7542 | 60/206,201 | 05/22/2000 |
| SEQ ID NO:7543 | 60/206,201 | 05/22/2000 |
| SEQ ID NO:7544 | 60/206,201 | 05/22/2000 |
| SEQ ID NO:7545 | 60/206,201 | 05/22/2000 |
| SEQ ID NO:7546 | 60/206,201 | 05/22/2000 |
| SEQ ID NO:7547 | 60/206,201 | 05/22/2000 |
| SEQ ID NO:7548 | 60/206,201 | 05/22/2000 |
| SEQ ID NO:7549 | 60/206,201 | 05/22/2000 |
| SEQ ID NO:7550 | 60/206,201 | 05/22/2000 |
| SEQ ID NO:7551 | 60/206,201 | 05/22/2000 |
| SEQ ID NO:7552 | 60/206,201 | 05/22/2000 |
| SEQ ID NO:7553 | 60/206,201 | 05/22/2000 |
| SEQ ID NO:7554 | 60/206,201 | 05/22/2000 |
| SEQ ID NO:7555 | 60/206,201 | 05/22/2000 |
| SEQ ID NO:7556 | 60/206,201 | 05/22/2000 |
| SEQ ID NO:7557 | 60/206,201 | 05/22/2000 |
| SEQ ID NO:7558 | 60/206,201 | 05/22/2000 |
| SEQ ID NO:7559 | 60/206,201 | 05/22/2000 |
| SEQ ID NO:7560 | 60/206,201 | 05/22/2000 |
| SEQ ID NO:7561 | 60/206,201 | 05/22/2000 |
| SEQ ID NO:7562 | 60/206,201 | 05/22/2000 |
| SEQ ID NO:7563 | 60/206,201 | 05/22/2000 |
| SEQ ID NO:7564 | 60/206,201 | 05/22/2000 |
| SEQ ID NO:7565 | 60/206,201 | 05/22/2000 |
| SEQ ID NO:7566 | 60/206,201 | 05/22/2000 |
| SEQ ID NO:7567 | 60/206,201 | 05/22/2000 |
| SEQ ID NO:7568 | 60/206,201 | 05/22/2000 |
| SEQ ID NO:7569 | 60/206,201 | 05/22/2000 |
| SEQ ID NO:7570 | 60/206,201 | 05/22/2000 |
| SEQ ID NO:7571 | 60/206,201 | 05/22/2000 |
| SEQ ID NO:7572 | 60/206,201 | 05/22/2000 |
| SEQ ID NO:7573 | 60/206,201 | 05/22/2000 |
| SEQ ID NO:7574 | 60/206,201 | 05/22/2000 |
| SEQ ID NO:7575 | 60/206,201 | 05/22/2000 |
| SEQ ID NO:7576 | 60/206,201 | 05/22/2000 |
| SEQ ID NO:7577 | 60/206,201 | 05/22/2000 |
| SEQ ID NO:7578 | 60/206,201 | 05/22/2000 |
| SEQ ID NO:7579 | 60/206,201 | 05/22/2000 |
| SEQ ID NO:7580 | 60/206,201 | 05/22/2000 |
| SEQ ID NO:7581 | 60/206,201 | 05/22/2000 |
| SEQ ID NO:7582 | 60/206,201 | 05/22/2000 |
| SEQ ID NO:7583 | 60/206,201 | 05/22/2000 |
| SEQ ID NO:7584 | 60/206,201 | 05/22/2000 |
| SEQ ID NO:7585 | 60/206,201 | 05/22/2000 |
| SEQ ID NO:7586 | 60/206,201 | 05/22/2000 |
| SEQ ID NO:7587 | 60/206,201 | 05/22/2000 |
| SEQ ID NO:7588 | 60/206,201 | 05/22/2000 |
| SEQ ID NO:7589 | 60/206,201 | 05/22/2000 |
| SEQ ID NO:7590 | 60/206,201 | 05/22/2000 |
| SEQ ID NO:7591 | 60/206,201 | 05/22/2000 |
| SEQ ID NO:7592 | 60/206,201 | 05/22/2000 |
| SEQ ID NO:7593 | 60/206,201 | 05/22/2000 |
| SEQ ID NO:7594 | 60/206,201 | 05/22/2000 |
| SEQ ID NO:7595 | 60/206,201 | 05/22/2000 |
| SEQ ID NO:7596 | 60/206,201 | 05/22/2000 |
| SEQ ID NO:7597 | 60/206,201 | 05/22/2000 |
| SEQ ID NO:7598 | 60/206,201 | 05/22/2000 |
| SEQ ID NO:7599 | 60/206,201 | 05/22/2000 |
| SEQ ID NO:7600 | 60/206,201 | 05/22/2000 |
| SEQ ID NO:7601 | 60/206,201 | 05/22/2000 |
| SEQ ID NO:7602 | 60/206,201 | 05/22/2000 |
| SEQ ID NO:7603 | 60/206,201 | 05/22/2000 |
| SEQ ID NO:7604 | 60/206,201 | 05/22/2000 |
| SEQ ID NO:7605 | 60/206,201 | 05/22/2000 |
| SEQ ID NO:7606 | 60/206,201 | 05/22/2000 |
| SEQ ID NO:7607 | 60/206,201 | 05/22/2000 |
| SEQ ID NO:7608 | 60/206,201 | 05/22/2000 |
| SEQ ID NO:7609 | 60/206,201 | 05/22/2000 |
| SEQ ID NO:7610 | 60/206,201 | 05/22/2000 |
| SEQ ID NO:7611 | 60/206,201 | 05/22/2000 |
| SEQ ID NO:7612 | 60/206,201 | 05/22/2000 |
| SEQ ID NO:7613 | 60/206,201 | 05/22/2000 |
| SEQ ID NO:7614 | 60/206,201 | 05/22/2000 |
| SEQ ID NO:7615 | 60/206,201 | 05/22/2000 |
| SEQ ID NO:7616 | 60/206,201 | 05/22/2000 |
| SEQ ID NO:7617 | 60/206,201 | 05/22/2000 |
| SEQ ID NO:7618 | 60/206,201 | 05/22/2000 |
| SEQ ID NO:7619 | 60/206,201 | 05/22/2000 |
| SEQ ID NO:7620 | 60/206,201 | 05/22/2000 |
| SEQ ID NO:7621 | 60/206,201 | 05/22/2000 |
| SEQ ID NO:7622 | 60/206,201 | 05/22/2000 |
| SEQ ID NO:7623 | 60/206,201 | 05/22/2000 |
| SEQ ID NO:7624 | 60/206,201 | 05/22/2000 |
| SEQ ID NO:7625 | 60/206,201 | 05/22/2000 |
| SEQ ID NO:7626 | 60/206,201 | 05/22/2000 |
| SEQ ID NO:7627 | 60/206,201 | 05/22/2000 |
| SEQ ID NO:7628 | 60/206,201 | 05/22/2000 |
| SEQ ID NO:7629 | 60/206,201 | 05/22/2000 |
| SEQ ID NO:7630 | 60/206,201 | 05/22/2000 |
| SEQ ID NO:7631 | 60/206,201 | 05/22/2000 |
| SEQ ID NO:7632 | 60/206,201 | 05/22/2000 |
| SEQ ID NO:7633 | 60/206,201 | 05/22/2000 |
| SEQ ID NO:7634 | 60/206,201 | 05/22/2000 |
| SEQ ID NO:7635 | 60/206,201 | 05/22/2000 |
| SEQ ID NO:7636 | 60/206,201 | 05/22/2000 |
| SEQ ID NO:7637 | 60/206,201 | 05/22/2000 |
| SEQ ID NO:7638 | 60/206,201 | 05/22/2000 |
| SEQ ID NO:7639 | 60/206,201 | 05/22/2000 |
| SEQ ID NO:7640 | 60/206,201 | 05/22/2000 |
| SEQ ID NO:7641 | 60/206,201 | 05/22/2000 |
| SEQ ID NO:7642 | 60/206,201 | 05/22/2000 |
| SEQ ID NO:7643 | 60/206,201 | 05/22/2000 |
| SEQ ID NO:7644 | 60/206,201 | 05/22/2000 |
| SEQ ID NO:7645 | 60/206,201 | 05/22/2000 |
| SEQ ID NO:7646 | 60/206,201 | 05/22/2000 |
| SEQ ID NO:7647 | 60/206,201 | 05/22/2000 |
| SEQ ID NO:7648 | 60/206,201 | 05/22/2000 |
| SEQ ID NO:7649 | 60/206,201 | 05/22/2000 |
| SEQ ID NO:7650 | 60/206,201 | 05/22/2000 |
| SEQ ID NO:7651 | 60/206,201 | 05/22/2000 |
| SEQ ID NO:7652 | 60/206,201 | 05/22/2000 |
| SEQ ID NO:7653 | 60/206,201 | 05/22/2000 |
| SEQ ID NO:7654 | 60/206,201 | 05/22/2000 |
| SEQ ID NO:7655 | 60/206,201 | 05/22/2000 |
| SEQ ID NO:7656 | 60/206,201 | 05/22/2000 |
| SEQ ID NO:7657 | 60/206,201 | 05/22/2000 |
| SEQ ID NO:7658 | 60/206,201 | 05/22/2000 |
| SEQ ID NO:7659 | 60/206,201 | 05/22/2000 |
| SEQ ID NO:7660 | 60/206,201 | 05/22/2000 |
| SEQ ID NO:7661 | 60/206,201 | 05/22/2000 |
| SEQ ID NO:7662 | 60/206,201 | 05/22/2000 |
| SEQ ID NO:7663 | 60/206,201 | 05/22/2000 |
| SEQ ID NO:7664 | 60/206,201 | 05/22/2000 |
| SEQ ID NO:7665 | 60/206,201 | 05/22/2000 |
| SEQ ID NO:7666 | 60/206,201 | 05/22/2000 |
| SEQ ID NO:7667 | 60/206,201 | 05/22/2000 |
| SEQ ID NO:7668 | 60/206,201 | 05/22/2000 |
| SEQ ID NO:7669 | 60/206,201 | 05/22/2000 |
| SEQ ID NO:7670 | 60/206,201 | 05/22/2000 |
| SEQ ID NO:7671 | 60/206,201 | 05/22/2000 |
| SEQ ID NO:7672 | 60/206,201 | 05/22/2000 |
| SEQ ID NO:7673 | 60/206,201 | 05/22/2000 |
| SEQ ID NO:7674 | 60/206,201 | 05/22/2000 |
| SEQ ID NO:7675 | 60/206,201 | 05/22/2000 |
| SEQ ID NO:7676 | 60/206,201 | 05/22/2000 |
| SEQ ID NO:7677 | 60/206,201 | 05/22/2000 |
| SEQ ID NO:7678 | 60/206,201 | 05/22/2000 |
| SEQ ID NO:7679 | 60/206,201 | 05/22/2000 |
| SEQ ID NO:7680 | 60/206,201 | 05/22/2000 |
| SEQ ID NO:7681 | 60/206,201 | 05/22/2000 |
| SEQ ID NO:7682 | 60/206,201 | 05/22/2000 |
| SEQ ID NO:7683 | 60/206,201 | 05/22/2000 |
| SEQ ID NO:7684 | 60/206,201 | 05/22/2000 |
| SEQ ID NO:7685 | 60/206,201 | 05/22/2000 |
| SEQ ID NO:7686 | 60/206,201 | 05/22/2000 |
| SEQ ID NO:7687 | 60/206,201 | 05/22/2000 |
| SEQ ID NO:7688 | 60/206,201 | 05/22/2000 |
| SEQ ID NO:7689 | 60/206,201 | 05/22/2000 |
| SEQ ID NO:7690 | 60/206,201 | 05/22/2000 |
| SEQ ID NO:7691 | 60/206,201 | 05/22/2000 |
| SEQ ID NO:7692 | 60/206,201 | 05/22/2000 |
| SEQ ID NO:7693 | 60/206,201 | 05/22/2000 |
| SEQ ID NO:7694 | 60/206,201 | 05/22/2000 |
| SEQ ID NO:7695 | 60/206,201 | 05/22/2000 |
| SEQ ID NO:7696 | 60/206,201 | 05/22/2000 |
| SEQ ID NO:7697 | 60/206,201 | 05/22/2000 |
| SEQ ID NO:7698 | 60/206,201 | 05/22/2000 |
| SEQ ID NO:7699 | 60/206,201 | 05/22/2000 |
| SEQ ID NO:7700 | 60/206,201 | 05/22/2000 |
| SEQ ID NO:7701 | 60/206,201 | 05/22/2000 |
| SEQ ID NO:7702 | 60/206,201 | 05/22/2000 |
| SEQ ID NO:7703 | 60/206,201 | 05/22/2000 |
| SEQ ID NO:7704 | 60/206,201 | 05/22/2000 |
| SEQ ID NO:7705 | 60/206,201 | 05/22/2000 |
| SEQ ID NO:7706 | 60/206,201 | 05/22/2000 |
| SEQ ID NO:7707 | 60/206,201 | 05/22/2000 |
| SEQ ID NO:7708 | 60/206,201 | 05/22/2000 |
| SEQ ID NO:7709 | 60/206,201 | 05/22/2000 |
| SEQ ID NO:7710 | 60/206,201 | 05/22/2000 |
| SEQ ID NO:7711 | 60/206,201 | 05/22/2000 |
| SEQ ID NO:7712 | 60/206,201 | 05/22/2000 |
| SEQ ID NO:7713 | 60/206,201 | 05/22/2000 |
| SEQ ID NO:7714 | 60/206,201 | 05/22/2000 |
| SEQ ID NO:7715 | 60/206,201 | 05/22/2000 |
| SEQ ID NO:7716 | 60/206,201 | 05/22/2000 |
| SEQ ID NO:7717 | 60/206,201 | 05/22/2000 |
| SEQ ID NO:7718 | 60/206,201 | 05/22/2000 |
| SEQ ID NO:7719 | 60/206,201 | 05/22/2000 |
| SEQ ID NO:7720 | 60/206,201 | 05/22/2000 |
| SEQ ID NO:7721 | 60/206,201 | 05/22/2000 |
| SEQ ID NO:7722 | 60/206,201 | 05/22/2000 |
| SEQ ID NO:7723 | 60/206,201 | 05/22/2000 |
| SEQ ID NO:7724 | 60/206,201 | 05/22/2000 |
| SEQ ID NO:7725 | 60/206,201 | 05/22/2000 |
| SEQ ID NO:7726 | 60/206,201 | 05/22/2000 |
| SEQ ID NO:7727 | 60/206,201 | 05/22/2000 |
| SEQ ID NO:7728 | 60/206,201 | 05/22/2000 |
| SEQ ID NO:7729 | 60/206,201 | 05/22/2000 |
| SEQ ID NO:7730 | 60/206,201 | 05/22/2000 |
| SEQ ID NO:7731 | 60/206,201 | 05/22/2000 |
| SEQ ID NO:7732 | 60/218,950 | 07/14/2000 |
| SEQ ID NO:7733 | 60/218,950 | 07/14/2000 |
| SEQ ID NO:7734 | 60/218,950 | 07/14/2000 |
| SEQ ID NO:7735 | 60/218,950 | 07/14/2000 |
| SEQ ID NO:7736 | 60/218,950 | 07/14/2000 |
| SEQ ID NO:7737 | 60/218,950 | 07/14/2000 |
| SEQ ID NO:7738 | 60/218,950 | 07/14/2000 |
| SEQ ID NO:7739 | 60/218,950 | 07/14/2000 |
| SEQ ID NO:7740 | 60/218,950 | 07/14/2000 |
| SEQ ID NO:7741 | 60/218,950 | 07/14/2000 |
| SEQ ID NO:7742 | 60/218,950 | 07/14/2000 |
| SEQ ID NO:7743 | 60/218,950 | 07/14/2000 |
| SEQ ID NO:7744 | 60/218,950 | 07/14/2000 |
| SEQ ID NO:7745 | 60/218,950 | 07/14/2000 |
| SEQ ID NO:7746 | 60/218,950 | 07/14/2000 |
| SEQ ID NO:7747 | 60/218,950 | 07/14/2000 |
| SEQ ID NO:7748 | 60/218,950 | 07/14/2000 |
| SEQ ID NO:7749 | 60/218,950 | 07/14/2000 |
| SEQ ID NO:7750 | 60/218,950 | 07/14/2000 |
| SEQ ID NO:7751 | 60/218,950 | 07/14/2000 |
| SEQ ID NO:7752 | 60/218,950 | 07/14/2000 |
| SEQ ID NO:7753 | 60/218,950 | 07/14/2000 |
| SEQ ID NO:7754 | 60/218,950 | 07/14/2000 |
| SEQ ID NO:7755 | 60/218,950 | 07/14/2000 |
| SEQ ID NO:7756 | 60/218,950 | 07/14/2000 |
| SEQ ID NO:7757 | 60/218,950 | 07/14/2000 |
| SEQ ID NO:7758 | 60/218,950 | 07/14/2000 |
| SEQ ID NO:7759 | 60/218,950 | 07/14/2000 |
| SEQ ID NO:7760 | 60/218,950 | 07/14/2000 |
| SEQ ID NO:7761 | 60/218,950 | 07/14/2000 |
| SEQ ID NO:7762 | 60/218,950 | 07/14/2000 |
| SEQ ID NO:7763 | 60/218,950 | 07/14/2000 |
| SEQ ID NO:7764 | 60/218,950 | 07/14/2000 |
| SEQ ID NO:7765 | 60/218,950 | 07/14/2000 |
| SEQ ID NO:7766 | 60/218,950 | 07/14/2000 |
| SEQ ID NO:7767 | 60/218,950 | 07/14/2000 |
| SEQ ID NO:7768 | 60/218,950 | 07/14/2000 |
| SEQ ID NO:7769 | 60/218,950 | 07/14/2000 |
| SEQ ID NO:7770 | 60/218,950 | 07/14/2000 |
| SEQ ID NO:7771 | 60/218,950 | 07/14/2000 |
| SEQ ID NO:7772 | 60/218,950 | 07/14/2000 |
| SEQ ID NO:7773 | 60/218,950 | 07/14/2000 |
| SEQ ID NO:7774 | 60/218,950 | 07/14/2000 |
| SEQ ID NO:7775 | 60/218,950 | 07/14/2000 |
| SEQ ID NO:7776 | 60/218,950 | 07/14/2000 |
| SEQ ID NO:7777 | 60/218,950 | 07/14/2000 |
| SEQ ID NO:7778 | 60/218,950 | 07/14/2000 |
| SEQ ID NO:7779 | 60/218,950 | 07/14/2000 |
| SEQ ID NO:7780 | 60/218,950 | 07/14/2000 |
| SEQ ID NO:7781 | 60/218,950 | 07/14/2000 |
| SEQ ID NO:7782 | 60/218,950 | 07/14/2000 |
| SEQ ID NO:7783 | 60/218,950 | 07/14/2000 |
| SEQ ID NO:7784 | 60/218,950 | 07/14/2000 |
| SEQ ID NO:7785 | 60/218,950 | 07/14/2000 |
| SEQ ID NO:7786 | 60/218,950 | 07/14/2000 |
| SEQ ID NO:7787 | 60/218,950 | 07/14/2000 |
| SEQ ID NO:7788 | 60/218,950 | 07/14/2000 |
| SEQ ID NO:7789 | 60/218,950 | 07/14/2000 |
| SEQ ID NO:7790 | 60/218,950 | 07/14/2000 |
| SEQ ID NO:7791 | 60/218,950 | 07/14/2000 |
| SEQ ID NO:7792 | 60/218,950 | 07/14/2000 |
| SEQ ID NO:7793 | 60/218,950 | 07/14/2000 |
| SEQ ID NO:7794 | 60/218,950 | 07/14/2000 |
| SEQ ID NO:7795 | 60/218,950 | 07/14/2000 |
| SEQ ID NO:7796 | 60/218,950 | 07/14/2000 |
| SEQ ID NO:7797 | 60/218,950 | 07/14/2000 |
| SEQ ID NO:7798 | 60/218,950 | 07/14/2000 |
| SEQ ID NO:7799 | 60/218,950 | 07/14/2000 |
| SEQ ID NO:7800 | 60/218,950 | 07/14/2000 |
| SEQ ID NO:7801 | 60/218,950 | 07/14/2000 |
| SEQ ID NO:7802 | 60/218,950 | 07/14/2000 |
| SEQ ID NO:7803 | 60/218,950 | 07/14/2000 |
| SEQ ID NO:7804 | 60/218,950 | 07/14/2000 |
| SEQ ID NO:7805 | 60/218,950 | 07/14/2000 |
| SEQ ID NO:7806 | 60/218,950 | 07/14/2000 |
| SEQ ID NO:7807 | 60/218,950 | 07/14/2000 |
| SEQ ID NO:7808 | 60/218,950 | 07/14/2000 |
| SEQ ID NO:7809 | 60/218,950 | 07/14/2000 |
| SEQ ID NO:7810 | 60/218,950 | 07/14/2000 |
| SEQ ID NO:7811 | 60/218,950 | 07/14/2000 |
| SEQ ID NO:7812 | 60/218,950 | 07/14/2000 |
| SEQ ID NO:7813 | 60/218,950 | 07/14/2000 |
| SEQ ID NO:7814 | 60/218,950 | 07/14/2000 |
| SEQ ID NO:7815 | 60/218,950 | 07/14/2000 |
| SEQ ID NO:7816 | 60/218,950 | 07/14/2000 |
| SEQ ID NO:7817 | 60/218,950 | 07/14/2000 |
| SEQ ID NO:7818 | 60/218,950 | 07/14/2000 |
| SEQ ID NO:7819 | 60/218,950 | 07/14/2000 |
| SEQ ID NO:7820 | 60/218,950 | 07/14/2000 |
| SEQ ID NO:7821 | 60/218,950 | 07/14/2000 |
| SEQ ID NO:7822 | 60/218,950 | 07/14/2000 |
| SEQ ID NO:7823 | 60/218,950 | 07/14/2000 |
| SEQ ID NO:7824 | 60/218,950 | 07/14/2000 |
| SEQ ID NO:7825 | 60/218,950 | 07/14/2000 |
| SEQ ID NO:7826 | 60/218,950 | 07/14/2000 |
| SEQ ID NO:7827 | 60/218,950 | 07/14/2000 |
| SEQ ID NO:7828 | 60/218,950 | 07/14/2000 |
| SEQ ID NO:7829 | 60/218,950 | 07/14/2000 |
| SEQ ID NO:7830 | 60/218,950 | 07/14/2000 |
| SEQ ID NO:7831 | 60/218,950 | 07/14/2000 |
| SEQ ID NO:7832 | 60/218,950 | 07/14/2000 |
| SEQ ID NO:7833 | 60/218,950 | 07/14/2000 |
| SEQ ID NO:7834 | 60/218,950 | 07/14/2000 |
| SEQ ID NO:7835 | 60/218,950 | 07/14/2000 |
| SEQ ID NO:7836 | 60/218,950 | 07/14/2000 |
| SEQ ID NO:7837 | 60/218,950 | 07/14/2000 |
| SEQ ID NO:7838 | 60/218,950 | 07/14/2000 |
| SEQ ID NO:7839 | 60/218,950 | 07/14/2000 |
| SEQ ID NO:7840 | 60/218,950 | 07/14/2000 |
| SEQ ID NO:7841 | 60/218,950 | 07/14/2000 |
| SEQ ID NO:7842 | 60/218,950 | 07/14/2000 |
| SEQ ID NO:7843 | 60/218,950 | 07/14/2000 |
| SEQ ID NO:7844 | 60/218,950 | 07/14/2000 |
| SEQ ID NO:7845 | 60/218,950 | 07/14/2000 |
| SEQ ID NO:7846 | 60/218,950 | 07/14/2000 |
| SEQ ID NO:7847 | 60/218,950 | 07/14/2000 |
| SEQ ID NO:7848 | 60/218,950 | 07/14/2000 |
| SEQ ID NO:7849 | 60/218,950 | 07/14/2000 |
| SEQ ID NO:7850 | 60/218,950 | 07/14/2000 |
| SEQ ID NO:7851 | 60/218,950 | 07/14/2000 |
| SEQ ID NO:7852 | 60/218,950 | 07/14/2000 |
| SEQ ID NO:7853 | 60/218,950 | 07/14/2000 |
| SEQ ID NO:7854 | 60/218,950 | 07/14/2000 |
| SEQ ID NO:7855 | 60/218,950 | 07/14/2000 |
| SEQ ID NO:7856 | 60/218,950 | 07/14/2000 |
| SEQ ID NO:7857 | 60/218,950 | 07/14/2000 |
| SEQ ID NO:7858 | 60/218,950 | 07/14/2000 |
| SEQ ID NO:7859 | 60/218,950 | 07/14/2000 |
| SEQ ID NO:7860 | 60/218,950 | 07/14/2000 |
| SEQ ID NO:7861 | 60/218,950 | 07/14/2000 |
| SEQ ID NO:7862 | 60/218,950 | 07/14/2000 |
| SEQ ID NO:7863 | 60/218,950 | 07/14/2000 |
| SEQ ID NO:7864 | 60/218,950 | 07/14/2000 |
| SEQ ID NO:7865 | 60/218,950 | 07/14/2000 |
| SEQ ID NO:7866 | 60/218,950 | 07/14/2000 |
| SEQ ID NO:7867 | 60/218,950 | 07/14/2000 |
| SEQ ID NO:7868 | 60/218,950 | 07/14/2000 |
| SEQ ID NO:7869 | 60/218,950 | 07/14/2000 |
| SEQ ID NO:7870 | 60/218,950 | 07/14/2000 |
| SEQ ID NO:7871 | 60/218,950 | 07/14/2000 |
| SEQ ID NO:7872 | 60/218,950 | 07/14/2000 |
| SEQ ID NO:7873 | 60/218,950 | 07/14/2000 |
| SEQ ID NO:7874 | 60/218,950 | 07/14/2000 |
| SEQ ID NO:7875 | 60/218,950 | 07/14/2000 |
| SEQ ID NO:7876 | 60/218,950 | 07/14/2000 |
| SEQ ID NO:7877 | 60/218,950 | 07/14/2000 |
| SEQ ID NO:7878 | 60/218,950 | 07/14/2000 |
| SEQ ID NO:7879 | 60/218,950 | 07/14/2000 |
| SEQ ID NO:7880 | 60/218,950 | 07/14/2000 |
| SEQ ID NO:7881 | 60/218,950 | 07/14/2000 |
| SEQ ID NO:7882 | 60/218,950 | 07/14/2000 |
| SEQ ID NO:7883 | 60/218,950 | 07/14/2000 |
| SEQ ID NO:7884 | 60/218,950 | 07/14/2000 |
| SEQ ID NO:7885 | 60/218,950 | 07/14/2000 |
| SEQ ID NO:7886 | 60/218,950 | 07/14/2000 |
| SEQ ID NO:7887 | 60/218,950 | 07/14/2000 |
| SEQ ID NO:7888 | 60/218,950 | 07/14/2000 |
| SEQ ID NO:7889 | 60/218,950 | 07/14/2000 |
| SEQ ID NO:7890 | 60/218,950 | 07/14/2000 |
| SEQ ID NO:7891 | 60/218,950 | 07/14/2000 |
| SEQ ID NO:7892 | 60/218,950 | 07/14/2000 |
| SEQ ID NO:7893 | 60/218,950 | 07/14/2000 |
| SEQ ID NO:7894 | 60/218,950 | 07/14/2000 |
| SEQ ID NO:7895 | 60/218,950 | 07/14/2000 |
| SEQ ID NO:7896 | 60/218,950 | 07/14/2000 |
| SEQ ID NO:7897 | 60/218,950 | 07/14/2000 |
| SEQ ID NO:7898 | 60/218,950 | 07/14/2000 |
| SEQ ID NO:7899 | 60/218,950 | 07/14/2000 |
| SEQ ID NO:7900 | 60/218,950 | 07/14/2000 |
| SEQ ID NO:7901 | 60/218,950 | 07/14/2000 |
| SEQ ID NO:7902 | 60/218,950 | 07/14/2000 |
| SEQ ID NO:7903 | 60/218,950 | 07/14/2000 |
| SEQ ID NO:7904 | 60/218,950 | 07/14/2000 |
| SEQ ID NO:7905 | 60/218,950 | 07/14/2000 |
| SEQ ID NO:7906 | 60/218,950 | 07/14/2000 |
| SEQ ID NO:7907 | 60/218,950 | 07/14/2000 |
| SEQ ID NO:7908 | 60/218,950 | 07/14/2000 |
| SEQ ID NO:7909 | 60/218,950 | 07/14/2000 |
| SEQ ID NO:7910 | 60/218,950 | 07/14/2000 |
| SEQ ID NO:7911 | 60/218,950 | 07/14/2000 |
| SEQ ID NO:7912 | 60/218,950 | 07/14/2000 |
| SEQ ID NO:7913 | 60/218,950 | 07/14/2000 |
| SEQ ID NO:7914 | 60/218,950 | 07/14/2000 |
| SEQ ID NO:7915 | 60/218,950 | 07/14/2000 |
| SEQ ID NO:7916 | 60/218,950 | 07/14/2000 |
| SEQ ID NO:7917 | 60/218,950 | 07/14/2000 |
| SEQ ID NO:7918 | 60/218,950 | 07/14/2000 |
| SEQ ID NO:7919 | 60/218,950 | 07/14/2000 |
| SEQ ID NO:7920 | 60/218,950 | 07/14/2000 |
| SEQ ID NO:7921 | 60/218,950 | 07/14/2000 |
| SEQ ID NO:7922 | 60/218,950 | 07/14/2000 |
| SEQ ID NO:7923 | 60/218,950 | 07/14/2000 |
| SEQ ID NO:7924 | 60/218,950 | 07/14/2000 |
| SEQ ID NO:7925 | 60/218,950 | 07/14/2000 |
| SEQ ID NO:7926 | 60/218,950 | 07/14/2000 |
| SEQ ID NO:7927 | 60/218,950 | 07/14/2000 |
| SEQ ID NO:7928 | 60/218,950 | 07/14/2000 |
| SEQ ID NO:7929 | 60/218,950 | 07/14/2000 |
| SEQ ID NO:7930 | 60/218,950 | 07/14/2000 |
| SEQ ID NO:7931 | 60/218,950 | 07/14/2000 |
| SEQ ID NO:7932 | 60/218,950 | 07/14/2000 |
| SEQ ID NO:7933 | 60/218,950 | 07/14/2000 |
| SEQ ID NO:7934 | 60/218,950 | 07/14/2000 |
| SEQ ID NO:7935 | 60/218,950 | 07/14/2000 |
| SEQ ID NO:7936 | 60/218,950 | 07/14/2000 |
| SEQ ID NO:7937 | 60/218,950 | 07/14/2000 |
| SEQ ID NO:7938 | 60/218,950 | 07/14/2000 |
| SEQ ID NO:7939 | 60/218,950 | 07/14/2000 |
| SEQ ID NO:7940 | 60/218,950 | 07/14/2000 |
| SEQ ID NO:7941 | 60/218,950 | 07/14/2000 |
| SEQ ID NO:7942 | 60/218,950 | 07/14/2000 |
| SEQ ID NO:7943 | 60/218,950 | 07/14/2000 |
| SEQ ID NO:7944 | 60/218,950 | 07/14/2000 |
| SEQ ID NO:7945 | 60/218,950 | 07/14/2000 |
| SEQ ID NO:7946 | 60/218,950 | 07/14/2000 |
| SEQ ID NO:7947 | 60/218,950 | 07/14/2000 |
| SEQ ID NO:7948 | 60/218,950 | 07/14/2000 |
| SEQ ID NO:7949 | 60/218,950 | 07/14/2000 |
| SEQ ID NO:7950 | 60/218,950 | 07/14/2000 |
| SEQ ID NO:7951 | 60/218,950 | 07/14/2000 |
| SEQ ID NO:7952 | 60/218,950 | 07/14/2000 |
| SEQ ID NO:7953 | 60/218,950 | 07/14/2000 |
| SEQ ID NO:7954 | 60/218,950 | 07/14/2000 |
| SEQ ID NO:7955 | 60/218,950 | 07/14/2000 |
| SEQ ID NO:7956 | 60/218,950 | 07/14/2000 |
| SEQ ID NO:7957 | 60/218,950 | 07/14/2000 |
| SEQ ID NO:7958 | 60/218,950 | 07/14/2000 |
| SEQ ID NO:7959 | 60/218,950 | 07/14/2000 |
| SEQ ID NO:7960 | 60/218,950 | 07/14/2000 |
| SEQ ID NO:7961 | 60/218,950 | 07/14/2000 |
| SEQ ID NO:7962 | 60/218,950 | 07/14/2000 |
| SEQ ID NO:7963 | 60/218,950 | 07/14/2000 |
| SEQ ID NO:7964 | 60/218,950 | 07/14/2000 |
| SEQ ID NO:7965 | 60/218,950 | 07/14/2000 |
| SEQ ID NO:7966 | 60/218,950 | 07/14/2000 |
| SEQ ID NO:7967 | 60/218,950 | 07/14/2000 |
| SEQ ID NO:7968 | 60/218,950 | 07/14/2000 |
| SEQ ID NO:7969 | 60/218,950 | 07/14/2000 |
| SEQ ID NO:7970 | 60/218,950 | 07/14/2000 |
| SEQ ID NO:7971 | 60/218,950 | 07/14/2000 |
| SEQ ID NO:7972 | 60/218,950 | 07/14/2000 |
| SEQ ID NO:7973 | 60/218,950 | 07/14/2000 |
| SEQ ID NO:7974 | 60/218,950 | 07/14/2000 |
| SEQ ID NO:7975 | 60/218,950 | 07/14/2000 |
| SEQ ID NO:7976 | 60/218,950 | 07/14/2000 |
| SEQ ID NO:7977 | 60/218,950 | 07/14/2000 |
| SEQ ID NO:7978 | 60/218,950 | 07/14/2000 |
| SEQ ID NO:7979 | 60/218,950 | 07/14/2000 |
| SEQ ID NO:7980 | 60/218,950 | 07/14/2000 |
| SEQ ID NO:7981 | 60/218,950 | 07/14/2000 |
| SEQ ID NO:7982 | 60/218,950 | 07/14/2000 |
| SEQ ID NO:7983 | 60/218,950 | 07/14/2000 |
| SEQ ID NO:7984 | 60/218,950 | 07/14/2000 |
| SEQ ID NO:7985 | 60/218,950 | 07/14/2000 |
| SEQ ID NO:7986 | 60/218,950 | 07/14/2000 |
| SEQ ID NO:7987 | 60/218,950 | 07/14/2000 |
| SEQ ID NO:7988 | 60/218,950 | 07/14/2000 |
| SEQ ID NO:7989 | 60/218,950 | 07/14/2000 |
| SEQ ID NO:7990 | 60/218,950 | 07/14/2000 |
| SEQ ID NO:7991 | 60/218,950 | 07/14/2000 |
| SEQ ID NO:7992 | 60/218,950 | 07/14/2000 |
| SEQ ID NO:7993 | 60/218,950 | 07/14/2000 |
| SEQ ID NO:7994 | 60/218,950 | 07/14/2000 |
| SEQ ID NO:7995 | 60/218,950 | 07/14/2000 |
| SEQ ID NO:7996 | 60/218,950 | 07/14/2000 |
| SEQ ID NO:7997 | 60/218,950 | 07/14/2000 |
| SEQ ID NO:7998 | 60/218,950 | 07/14/2000 |
| SEQ ID NO:7999 | 60/218,950 | 07/14/2000 |
| SEQ ID NO:8000 | 60/218,950 | 07/14/2000 |
| SEQ ID NO:8001 | 60/218,950 | 07/14/2000 |
| SEQ ID NO:8002 | 60/218,950 | 07/14/2000 |
| SEQ ID NO:8003 | 60/218,950 | 07/14/2000 |
| SEQ ID NO:8004 | 60/218,950 | 07/14/2000 |
| SEQ ID NO:8005 | 60/218,950 | 07/14/2000 |
| SEQ ID NO:8006 | 60/218,950 | 07/14/2000 |
| SEQ ID NO:8007 | 60/218,950 | 07/14/2000 |
| SEQ ID NO:8008 | 60/218,950 | 07/14/2000 |
| SEQ ID NO:8009 | 60/218,950 | 07/14/2000 |
| SEQ ID NO:8010 | 60/218,950 | 07/14/2000 |
| SEQ ID NO:8011 | 60/218,950 | 07/14/2000 |
| SEQ ID NO:8012 | 60/218,950 | 07/14/2000 |
| SEQ ID NO:8013 | 60/218,950 | 07/14/2000 |
| SEQ ID NO:8014 | 60/218,950 | 07/14/2000 |
| SEQ ID NO:8015 | 60/218,950 | 07/14/2000 |
| SEQ ID NO:8016 | 60/218,950 | 07/14/2000 |
| SEQ ID NO:8017 | 60/218,950 | 07/14/2000 |
| SEQ ID NO:8018 | 60/218,950 | 07/14/2000 |
| SEQ ID NO:8019 | 60/218,950 | 07/14/2000 |
| SEQ ID NO:8020 | 60/218,950 | 07/14/2000 |
| SEQ ID NO:8021 | 60/218,950 | 07/14/2000 |
| SEQ ID NO:8022 | 60/218,950 | 07/14/2000 |
| SEQ ID NO:8023 | 60/218,950 | 07/14/2000 |
| SEQ ID NO:8024 | 60/218,950 | 07/14/2000 |
| SEQ ID NO:8025 | 60/218,950 | 07/14/2000 |
| SEQ ID NO:8026 | 60/218,950 | 07/14/2000 |
| SEQ ID NO:8027 | 60/218,950 | 07/14/2000 |
| SEQ ID NO:8028 | 60/218,950 | 07/14/2000 |
| SEQ ID NO:8029 | 60/218,950 | 07/14/2000 |
| SEQ ID NO:8030 | 60/218,950 | 07/14/2000 |
| SEQ ID NO:8031 | 60/218,950 | 07/14/2000 |
| SEQ ID NO:8032 | 60/218,950 | 07/14/2000 |
| SEQ ID NO:8033 | 60/218,950 | 07/14/2000 |
| SEQ ID NO:8034 | 60/218,950 | 07/14/2000 |
| SEQ ID NO:8035 | 60/218,950 | 07/14/2000 |
| SEQ ID NO:8036 | 60/218,950 | 07/14/2000 |
| SEQ ID NO:8037 | 60/218,950 | 07/14/2000 |
| SEQ ID NO:8038 | 60/218,950 | 07/14/2000 |
| SEQ ID NO:8039 | 60/218,950 | 07/14/2000 |
| SEQ ID NO:8040 | 60/218,950 | 07/14/2000 |
| SEQ ID NO:8041 | 60/218,950 | 07/14/2000 |
| SEQ ID NO:8042 | 60/218,950 | 07/14/2000 |
| SEQ ID NO:8043 | 60/218,950 | 07/14/2000 |
| SEQ ID NO:8044 | 60/218,950 | 07/14/2000 |
| SEQ ID NO:8045 | 60/218,950 | 07/14/2000 |
| SEQ ID NO:8046 | 60/218,950 | 07/14/2000 |
| SEQ ID NO:8047 | 60/218,950 | 07/14/2000 |
| SEQ ID NO:8048 | 60/218,950 | 07/14/2000 |
| SEQ ID NO:8049 | 60/218,950 | 07/14/2000 |
| SEQ ID NO:8050 | 60/218,950 | 07/14/2000 |
| SEQ ID NO:8051 | 60/218,950 | 07/14/2000 |
| SEQ ID NO:8052 | 60/218,950 | 07/14/2000 |
| SEQ ID NO:8053 | 60/218,950 | 07/14/2000 |
| SEQ ID NO:8054 | 60/218,950 | 07/14/2000 |
| SEQ ID NO:8055 | 60/218,950 | 07/14/2000 |
| SEQ ID NO:8056 | 60/218,950 | 07/14/2000 |
| SEQ ID NO:8057 | 60/218,950 | 07/14/2000 |
| SEQ ID NO:8058 | 60/218,950 | 07/14/2000 |
| SEQ ID NO:8059 | 60/218,950 | 07/14/2000 |
| SEQ ID NO:8060 | 60/218,950 | 07/14/2000 |
| SEQ ID NO:8061 | 60/218,950 | 07/14/2000 |
| SEQ ID NO:8062 | 60/218,950 | 07/14/2000 |
| SEQ ID NO:8063 | 60/218,950 | 07/14/2000 |
| SEQ ID NO:8064 | 60/218,950 | 07/14/2000 |
| SEQ ID NO:8065 | 60/218,950 | 07/14/2000 |
| SEQ ID NO:8066 | 60/218,950 | 07/14/2000 |
| SEQ ID NO:8067 | 60/218,950 | 07/14/2000 |
| SEQ ID NO:8068 | 60/218,950 | 07/14/2000 |
| SEQ ID NO:8069 | 60/218,950 | 07/14/2000 |
| SEQ ID NO:8070 | 60/218,950 | 07/14/2000 |
| SEQ ID NO:8071 | 60/218,950 | 07/14/2000 |
| SEQ ID NO:8072 | 60/218,950 | 07/14/2000 |
| SEQ ID NO:8073 | 60/218,950 | 07/14/2000 |
| SEQ ID NO:8074 | 60/218,950 | 07/14/2000 |
| SEQ ID NO:8075 | 60/218,950 | 07/14/2000 |
| SEQ ID NO:8076 | 60/218,950 | 07/14/2000 |
| SEQ ID NO:8077 | 60/218,950 | 07/14/2000 |
| SEQ ID NO:8078 | 60/218,950 | 07/14/2000 |
| SEQ ID NO:8079 | 60/218,950 | 07/14/2000 |
| SEQ ID NO:8080 | 60/218,950 | 07/14/2000 |
| SEQ ID NO:8081 | 60/218,950 | 07/14/2000 |
| SEQ ID NO:8082 | 60/218,950 | 07/14/2000 |
| SEQ ID NO:8083 | 60/218,950 | 07/14/2000 |
| SEQ ID NO:8084 | 60/218,950 | 07/14/2000 |
| SEQ ID NO:8085 | 60/218,950 | 07/14/2000 |
| SEQ ID NO:8086 | 60/218,950 | 07/14/2000 |
| SEQ ID NO:8087 | 60/218,950 | 07/14/2000 |
| SEQ ID NO:8088 | 60/218,950 | 07/14/2000 |
| SEQ ID NO:8089 | 60/218,950 | 07/14/2000 |
| SEQ ID NO:8090 | 60/218,950 | 07/14/2000 |
| SEQ ID NO:8091 | 60/218,950 | 07/14/2000 |
| SEQ ID NO:8092 | 60/218,950 | 07/14/2000 |
| SEQ ID NO:8093 | 60/218,950 | 07/14/2000 |
| SEQ ID NO:8094 | 60/218,950 | 07/14/2000 |
| SEQ ID NO:8095 | 60/218,950 | 07/14/2000 |
| SEQ ID NO:8096 | 60/218,950 | 07/14/2000 |
| SEQ ID NO:8097 | 60/218,950 | 07/14/2000 |
| SEQ ID NO:8098 | 60/218,950 | 07/14/2000 |
| SEQ ID NO:8099 | 60/218,950 | 07/14/2000 |
| SEQ ID NO:8100 | 60/218,950 | 07/14/2000 |
| SEQ ID NO:8101 | 60/218,950 | 07/14/2000 |
| SEQ ID NO:8102 | 60/218,950 | 07/14/2000 |
| SEQ ID NO:8103 | 60/218,950 | 07/14/2000 |
| SEQ ID NO:8104 | 60/218,950 | 07/14/2000 |
| SEQ ID NO:8105 | 60/218,950 | 07/14/2000 |
| SEQ ID NO:8106 | 60/218,950 | 07/14/2000 |
| SEQ ID NO:8107 | 60/218,950 | 07/14/2000 |
| SEQ ID NO:8108 | 60/218,950 | 07/14/2000 |
| SEQ ID NO:8109 | 60/218,950 | 07/14/2000 |
| SEQ ID NO:8110 | 60/218,950 | 07/14/2000 |
| SEQ ID NO:8111 | 60/218,950 | 07/14/2000 |
| SEQ ID NO:8112 | 60/218,950 | 07/14/2000 |
| SEQ ID NO:8113 | 60/218,950 | 07/14/2000 |
| SEQ ID NO:8114 | 60/218,950 | 07/14/2000 |
| SEQ ID NO:8115 | 60/218,950 | 07/14/2000 |
| SEQ ID NO:8116 | 60/218,950 | 07/14/2000 |
| SEQ ID NO:8117 | 60/218,950 | 07/14/2000 |
| SEQ ID NO:8118 | 60/218,950 | 07/14/2000 |
| SEQ ID NO:8119 | 60/218,950 | 07/14/2000 |
| SEQ ID NO:8120 | 60/218,950 | 07/14/2000 |
| SEQ ID NO:8121 | 60/218,950 | 07/14/2000 |
| SEQ ID NO:8122 | 60/218,950 | 07/14/2000 |
| SEQ ID NO:8123 | 60/218,950 | 07/14/2000 |
| SEQ ID NO:8124 | 60/218,950 | 07/14/2000 |
| SEQ ID NO:8125 | 60/218,950 | 07/14/2000 |
| SEQ ID NO:8126 | 60/218,950 | 07/14/2000 |
| SEQ ID NO:8127 | 60/218,950 | 07/14/2000 |
| SEQ ID NO:8128 | 60/218,950 | 07/14/2000 |
| SEQ ID NO:8129 | 60/218,950 | 07/14/2000 |
| SEQ ID NO:8130 | 60/218,950 | 07/14/2000 |
| SEQ ID NO:8131 | 60/218,950 | 07/14/2000 |
| SEQ ID NO:8132 | 60/218,950 | 07/14/2000 |
| SEQ ID NO:8133 | 60/218,950 | 07/14/2000 |
| SEQ ID NO:8134 | 60/218,950 | 07/14/2000 |
| SEQ ID NO:8135 | 60/218,950 | 07/14/2000 |
| SEQ ID NO:8136 | 60/218,950 | 07/14/2000 |
| SEQ ID NO:8137 | 60/218,950 | 07/14/2000 |
| SEQ ID NO:8138 | 60/218,950 | 07/14/2000 |
| SEQ ID NO:8139 | 60/218,950 | 07/14/2000 |
| SEQ ID NO:8140 | 60/218,950 | 07/14/2000 |
| SEQ ID NO:8141 | 60/218,950 | 07/14/2000 |
| SEQ ID NO:8142 | 60/218,950 | 07/14/2000 |
| SEQ ID NO:8143 | 60/218,950 | 07/14/2000 |
| SEQ ID NO:8144 | 60/218,950 | 07/14/2000 |
| SEQ ID NO:8145 | 60/218,950 | 07/14/2000 |
| SEQ ID NO:8146 | 60/218,950 | 07/14/2000 |
| SEQ ID NO:8147 | 60/218,950 | 07/14/2000 |
| SEQ ID NO:8148 | 60/218,950 | 07/14/2000 |
| SEQ ID NO:8149 | 60/218,950 | 07/14/2000 |
| SEQ ID NO:8150 | 60/218,950 | 07/14/2000 |
| SEQ ID NO:8151 | 60/218,950 | 07/14/2000 |
| SEQ ID NO:8152 | 60/218,950 | 07/14/2000 |
| SEQ ID NO:8153 | 60/218,950 | 07/14/2000 |
| SEQ ID NO:8154 | 60/218,950 | 07/14/2000 |
| SEQ ID NO:8155 | 60/218,950 | 07/14/2000 |
| SEQ ID NO:8156 | 60/218,950 | 07/14/2000 |
| SEQ ID NO:8157 | 60/218,950 | 07/14/2000 |
| SEQ ID NO:8158 | 60/218,950 | 07/14/2000 |
| SEQ ID NO:8159 | 60/218,950 | 07/14/2000 |
| SEQ ID NO:8160 | 60/218,950 | 07/14/2000 |
| SEQ ID NO:8161 | 60/218,950 | 07/14/2000 |
| SEQ ID NO:8162 | 60/218,950 | 07/14/2000 |
| SEQ ID NO:8163 | 60/218,950 | 07/14/2000 |
| SEQ ID NO:8164 | 60/218,950 | 07/14/2000 |
| SEQ ID NO:8165 | 60/218,950 | 07/14/2000 |
| SEQ ID NO:8166 | 60/218,950 | 07/14/2000 |
| SEQ ID NO:8167 | 60/218,950 | 07/14/2000 |
| SEQ ID NO:8168 | 60/218,950 | 07/14/2000 |
| SEQ ID NO:8169 | 60/218,950 | 07/14/2000 |
| SEQ ID NO:8170 | 60/218,950 | 07/14/2000 |
| SEQ ID NO:8171 | 60/218,950 | 07/14/2000 |
| SEQ ID NO:8172 | 60/218,950 | 07/14/2000 |
| SEQ ID NO:8173 | 60/218,950 | 07/14/2000 |
| SEQ ID NO:8174 | 60/218,950 | 07/14/2000 |
| SEQ ID NO:8175 | 60/218,950 | 07/14/2000 |
| SEQ ID NO:8176 | 60/218,950 | 07/14/2000 |
| SEQ ID NO:8177 | 60/218,950 | 07/14/2000 |
| SEQ ID NO:8178 | 60/218,950 | 07/14/2000 |
| SEQ ID NO:8179 | 60/218,950 | 07/14/2000 |
| SEQ ID NO:8180 | 60/218,950 | 07/14/2000 |
| SEQ ID NO:8181 | 60/218,950 | 07/14/2000 |
| SEQ ID NO:8182 | 60/218,950 | 07/14/2000 |
| SEQ ID NO:8183 | 60/218,950 | 07/14/2000 |
| SEQ ID NO:8184 | 60/218,950 | 07/14/2000 |
| SEQ ID NO:8185 | 60/218,950 | 07/14/2000 |
| SEQ ID NO:8186 | 60/218,950 | 07/14/2000 |
| SEQ ID NO:8187 | 60/218,950 | 07/14/2000 |
| SEQ ID NO:8188 | 60/218,950 | 07/14/2000 |
| SEQ ID NO:8189 | 60/218,950 | 07/14/2000 |
| SEQ ID NO:8190 | 60/218,950 | 07/14/2000 |
| SEQ ID NO:8191 | 60/218,950 | 07/14/2000 |
| SEQ ID NO:8192 | 60/218,950 | 07/14/2000 |
| SEQ ID NO:8193 | 60/218,950 | 07/14/2000 |
| SEQ ID NO:8194 | 60/218,950 | 07/14/2000 |
| SEQ ID NO:8195 | 60/218,950 | 07/14/2000 |
| SEQ ID NO:8196 | 60/218,950 | 07/14/2000 |
| SEQ ID NO:8197 | 60/218,950 | 07/14/2000 |
| SEQ ID NO:8198 | 60/218,950 | 07/14/2000 |
| SEQ ID NO:8199 | 60/218,950 | 07/14/2000 |
| SEQ ID NO:8200 | 60/218,950 | 07/14/2000 |
| SEQ ID NO:8201 | 60/218,950 | 07/14/2000 |
| SEQ ID NO:8202 | 60/218,950 | 07/14/2000 |
| SEQ ID NO:8203 | 60/218,950 | 07/14/2000 |
| SEQ ID NO:8204 | 60/218,950 | 07/14/2000 |
| SEQ ID NO:8205 | 60/218,950 | 07/14/2000 |
| SEQ ID NO:8206 | 60/218,950 | 07/14/2000 |
| SEQ ID NO:8207 | 60/218,950 | 07/14/2000 |
| SEQ ID NO:8208 | 60/218,950 | 07/14/2000 |
| SEQ ID NO:8209 | 60/218,950 | 07/14/2000 |
| SEQ ID NO:8210 | 60/218,950 | 07/14/2000 |
| SEQ ID NO:8211 | 60/218,950 | 07/14/2000 |
| SEQ ID NO:8212 | 60/218,950 | 07/14/2000 |
| SEQ ID NO:8213 | 60/218,950 | 07/14/2000 |
| SEQ ID NO:8214 | 60/218,950 | 07/14/2000 |
| SEQ ID NO:8215 | 60/218,950 | 07/14/2000 |
| SEQ ID NO:8216 | 60/218,950 | 07/14/2000 |
| SEQ ID NO:8217 | 60/218,950 | 07/14/2000 |
| SEQ ID NO:8218 | 60/218,950 | 07/14/2000 |
| SEQ ID NO:8219 | 60/218,950 | 07/14/2000 |
| SEQ ID NO:8220 | 60/218,950 | 07/14/2000 |
| SEQ ID NO:8221 | 60/218,950 | 07/14/2000 |
| SEQ ID NO:8222 | 60/218,950 | 07/14/2000 |
| SEQ ID NO:8223 | 60/218,950 | 07/14/2000 |
| SEQ ID NO:8224 | 60/218,950 | 07/14/2000 |
| SEQ ID NO:8225 | 60/218,950 | 07/14/2000 |
| SEQ ID NO:8226 | 60/218,950 | 07/14/2000 |
| SEQ ID NO:8227 | 60/218,950 | 07/14/2000 |
| SEQ ID NO:8228 | 60/218,950 | 07/14/2000 |
| SEQ ID NO:8229 | 60/218,950 | 07/14/2000 |
| SEQ ID NO:8230 | 60/218,950 | 07/14/2000 |
| SEQ ID NO:8231 | 60/218,950 | 07/14/2000 |
| SEQ ID NO:8232 | 60/218,950 | 07/14/2000 |
| SEQ ID NO:8233 | 60/218,950 | 07/14/2000 |
| SEQ ID NO:8234 | 60/218,950 | 07/14/2000 |
| SEQ ID NO:8235 | 60/218,950 | 07/14/2000 |
| SEQ ID NO:8236 | 60/218,950 | 07/14/2000 |
| SEQ ID NO:8237 | 60/218,950 | 07/14/2000 |
| SEQ ID NO:8238 | 60/218,950 | 07/14/2000 |
| SEQ ID NO:8239 | 60/218,950 | 07/14/2000 |
| SEQ ID NO:8240 | 60/218,950 | 07/14/2000 |
| SEQ ID NO:8241 | 60/218,950 | 07/14/2000 |
| SEQ ID NO:8242 | 60/218,950 | 07/14/2000 |
| SEQ ID NO:8243 | 60/218,950 | 07/14/2000 |
| SEQ ID NO:8244 | 60/218,950 | 07/14/2000 |
| SEQ ID NO:8245 | 60/218,950 | 07/14/2000 |
| SEQ ID NO:8246 | 60/218,950 | 07/14/2000 |
| SEQ ID NO:8247 | 60/218,950 | 07/14/2000 |
| SEQ ID NO:8248 | 60/218,950 | 07/14/2000 |
| SEQ ID NO:8249 | 60/218,950 | 07/14/2000 |
| SEQ ID NO:8250 | 60/218,950 | 07/14/2000 |
| SEQ ID NO:8251 | 60/218,950 | 07/14/2000 |
| SEQ ID NO:8252 | 60/218,950 | 07/14/2000 |
| SEQ ID NO:8253 | 60/218,950 | 07/14/2000 |
| SEQ ID NO:8254 | 60/218,950 | 07/14/2000 |
| SEQ ID NO:8255 | 60/218,950 | 07/14/2000 |
| SEQ ID NO:8256 | 60/218,950 | 07/14/2000 |
| SEQ ID NO:8257 | 60/218,950 | 07/14/2000 |
| SEQ ID NO:8258 | 60/218,950 | 07/14/2000 |
| SEQ ID NO:8259 | 60/218,950 | 07/14/2000 |
| SEQ ID NO:8260 | 60/218,950 | 07/14/2000 |
| SEQ ID NO:8261 | 60/218,950 | 07/14/2000 |
| SEQ ID NO:8262 | 60/218,950 | 07/14/2000 |
| SEQ ID NO:8263 | 60/218,950 | 07/14/2000 |
| SEQ ID NO:8264 | 60/218,950 | 07/14/2000 |
| SEQ ID NO:8265 | 60/218,950 | 07/14/2000 |
| SEQ ID NO:8266 | 60/218,950 | 07/14/2000 |
| SEQ ID NO:8267 | 60/218,950 | 07/14/2000 |
| SEQ ID NO:8268 | 60/218,950 | 07/14/2000 |
| SEQ ID NO:8269 | 60/218,950 | 07/14/2000 |
| SEQ ID NO:8270 | 60/218,950 | 07/14/2000 |
| SEQ ID NO:8271 | 60/218,950 | 07/14/2000 |
| SEQ ID NO:8272 | 60/218,950 | 07/14/2000 |
| SEQ ID NO:8273 | 60/218,950 | 07/14/2000 |
| SEQ ID NO:8274 | 60/218,950 | 07/14/2000 |
| SEQ ID NO:8275 | 60/218,950 | 07/14/2000 |
| SEQ ID NO:8276 | 60/218,950 | 07/14/2000 |
| SEQ ID NO:8277 | 60/218,950 | 07/14/2000 |
| SEQ ID NO:8278 | 60/218,950 | 07/14/2000 |
| SEQ ID NO:8279 | 60/218,950 | 07/14/2000 |
| SEQ ID NO:8280 | 60/218,950 | 07/14/2000 |
| SEQ ID NO:8281 | 60/218,950 | 07/14/2000 |
| SEQ ID NO:8282 | 60/218,950 | 07/14/2000 |
| SEQ ID NO:8283 | 60/218,950 | 07/14/2000 |
| SEQ ID NO:8284 | 60/218,950 | 07/14/2000 |
| SEQ ID NO:8285 | 60/218,950 | 07/14/2000 |
| SEQ ID NO:8286 | 60/218,950 | 07/14/2000 |
| SEQ ID NO:8287 | 60/218,950 | 07/14/2000 |
| SEQ ID NO:8288 | 60/218,950 | 07/14/2000 |
| SEQ ID NO:8289 | 60/218,950 | 07/14/2000 |
| SEQ ID NO:8290 | 60/218,950 | 07/14/2000 |
| SEQ ID NO:8291 | 60/218,950 | 07/14/2000 |
| SEQ ID NO:8292 | 60/218,950 | 07/14/2000 |
| SEQ ID NO:8293 | 60/218,950 | 07/14/2000 |
| SEQ ID NO:8294 | 60/218,950 | 07/14/2000 |
| SEQ ID NO:8295 | 60/218,950 | 07/14/2000 |
| SEQ ID NO:8296 | 60/218,950 | 07/14/2000 |
| SEQ ID NO:8297 | 60/218,950 | 07/14/2000 |
| SEQ ID NO:8298 | 60/218,950 | 07/14/2000 |
| SEQ ID NO:8299 | 60/218,950 | 07/14/2000 |
| SEQ ID NO:8300 | 60/218,950 | 07/14/2000 |
| SEQ ID NO:8301 | 60/218,950 | 07/14/2000 |
| SEQ ID NO:8302 | 60/218,950 | 07/14/2000 |
| SEQ ID NO:8303 | 60/218,950 | 07/14/2000 |
| SEQ ID NO:8304 | 60/218,950 | 07/14/2000 |
| SEQ ID NO:8305 | 60/218,950 | 07/14/2000 |
| SEQ ID NO:8306 | 60/218,950 | 07/14/2000 |
| SEQ ID NO:8307 | 60/218,950 | 07/14/2000 |
| SEQ ID NO:8308 | 60/218,950 | 07/14/2000 |
| SEQ ID NO:8309 | 60/218,950 | 07/14/2000 |
| SEQ ID NO:8310 | 60/218,950 | 07/14/2000 |
| SEQ ID NO:8311 | 60/218,950 | 07/14/2000 |
| SEQ ID NO:8312 | 60/218,950 | 07/14/2000 |
| SEQ ID NO:8313 | 60/218,950 | 07/14/2000 |
| SEQ ID NO:8314 | 60/218,950 | 07/14/2000 |
| SEQ ID NO:8315 | 60/218,950 | 07/14/2000 |
| SEQ ID NO:8316 | 60/218,950 | 07/14/2000 |
| SEQ ID NO:8317 | 60/218,950 | 07/14/2000 |
| SEQ ID NO:8318 | 60/218,950 | 07/14/2000 |
| SEQ ID NO:8319 | 60/218,950 | 07/14/2000 |
| SEQ ID NO:8320 | 60/218,950 | 07/14/2000 |
| SEQ ID NO:8321 | 60/218,950 | 07/14/2000 |
| SEQ ID NO:8322 | 60/218,950 | 07/14/2000 |
| SEQ ID NO:8323 | 60/218,950 | 07/14/2000 |
| SEQ ID NO:8324 | 60/218,950 | 07/14/2000 |
| SEQ ID NO:8325 | 60/218,950 | 07/14/2000 |
| SEQ ID NO:8326 | 60/218,950 | 07/14/2000 |
| SEQ ID NO:8327 | 60/218,950 | 07/14/2000 |
| SEQ ID NO:8328 | 60/218,950 | 07/14/2000 |
| SEQ ID NO:8329 | 60/218,950 | 07/14/2000 |
| SEQ ID NO:8330 | 60/218,950 | 07/14/2000 |
| SEQ ID NO:8331 | 60/218,950 | 07/14/2000 |
| SEQ ID NO:8332 | 60/218,950 | 07/14/2000 |
| SEQ ID NO:8333 | 60/218,950 | 07/14/2000 |
| SEQ ID NO:8334 | 60/218,950 | 07/14/2000 |
| SEQ ID NO:8335 | 60/218,950 | 07/14/2000 |
| SEQ ID NO:8336 | 60/218,950 | 07/14/2000 |
| SEQ ID NO:8337 | 60/218,950 | 07/14/2000 |
| SEQ ID NO:8338 | 60/218,950 | 07/14/2000 |
| SEQ ID NO:8339 | 60/218,950 | 07/14/2000 |
| SEQ ID NO:8340 | 60/218,950 | 07/14/2000 |
| SEQ ID NO:8341 | 60/218,950 | 07/14/2000 |
| SEQ ID NO:8342 | 60/218,950 | 07/14/2000 |
| SEQ ID NO:8343 | 60/218,950 | 07/14/2000 |
| SEQ ID NO:8344 | 60/218,950 | 07/14/2000 |
| SEQ ID NO:8345 | 60/218,950 | 07/14/2000 |
| SEQ ID NO:8346 | 60/218,950 | 07/14/2000 |
| SEQ ID NO:8347 | 60/218,950 | 07/14/2000 |
| SEQ ID NO:8348 | 60/218,950 | 07/14/2000 |
| SEQ ID NO:8349 | 60/218,950 | 07/14/2000 |
| SEQ ID NO:8350 | 60/218,950 | 07/14/2000 |
| SEQ ID NO:8351 | 60/218,950 | 07/14/2000 |
| SEQ ID NO:8352 | 60/218,950 | 07/14/2000 |
| SEQ ID NO:8353 | 60/218,950 | 07/14/2000 |
| SEQ ID NO:8354 | 60/218,950 | 07/14/2000 |
| SEQ ID NO:8355 | 60/218,950 | 07/14/2000 |
| SEQ ID NO:8356 | 60/218,950 | 07/14/2000 |
| SEQ ID NO:8357 | 60/218,950 | 07/14/2000 |
| SEQ ID NO:8358 | 60/218,950 | 07/14/2000 |
| SEQ ID NO:8359 | 60/218,950 | 07/14/2000 |
| SEQ ID NO:8360 | 60/218,950 | 07/14/2000 |
| SEQ ID NO:8361 | 60/218,950 | 07/14/2000 |
| SEQ ID NO:8362 | 60/218,950 | 07/14/2000 |
| SEQ ID NO:8363 | 60/218,950 | 07/14/2000 |
| SEQ ID NO:8364 | 60/218,950 | 07/14/2000 |
| SEQ ID NO:8365 | 60/218,950 | 07/14/2000 |
| SEQ ID NO:8366 | 60/218,950 | 07/14/2000 |
| SEQ ID NO:8367 | 60/218,950 | 07/14/2000 |
| SEQ ID NO:8368 | 60/218,950 | 07/14/2000 |
| SEQ ID NO:8369 | 60/218,950 | 07/14/2000 |
| SEQ ID NO:8370 | 60/218,950 | 07/14/2000 |
| SEQ ID NO:8371 | 60/218,950 | 07/14/2000 |
| SEQ ID NO:8372 | 60/218,950 | 07/14/2000 |
| SEQ ID NO:8373 | 60/218,950 | 07/14/2000 |
| SEQ ID NO:8374 | 60/218,950 | 07/14/2000 |
| SEQ ID NO:8375 | 60/218,950 | 07/14/2000 |
| SEQ ID NO:8376 | 60/218,950 | 07/14/2000 |
| SEQ ID NO:8377 | 60/218,950 | 07/14/2000 |
| SEQ ID NO:8378 | 60/218,950 | 07/14/2000 |
| SEQ ID NO:8379 | 60/218,950 | 07/14/2000 |
| SEQ ID NO:8380 | 60/218,950 | 07/14/2000 |
| SEQ ID NO:8381 | 60/218,950 | 07/14/2000 |
| SEQ ID NO:8382 | 60/218,950 | 07/14/2000 |
| SEQ ID NO:8383 | 60/218,950 | 07/14/2000 |
| SEQ ID NO:8384 | 60/218,950 | 07/14/2000 |
| SEQ ID NO:8385 | 60/218,950 | 07/14/2000 |
| SEQ ID NO:8386 | 60/218,950 | 07/14/2000 |
| SEQ ID NO:8387 | 60/218,950 | 07/14/2000 |
| SEQ ID NO:8388 | 60/218,950 | 07/14/2000 |
| SEQ ID NO:8389 | 60/218,950 | 07/14/2000 |
| SEQ ID NO:8390 | 60/218,950 | 07/14/2000 |
| SEQ ID NO:8391 | 60/218,950 | 07/14/2000 |
| SEQ ID NO:8392 | 60/218,950 | 07/14/2000 |
| SEQ ID NO:8393 | 60/218,950 | 07/14/2000 |
| SEQ ID NO:8394 | 60/218,950 | 07/14/2000 |
| SEQ ID NO:8395 | 60/218,950 | 07/14/2000 |
| SEQ ID NO:8396 | 60/218,950 | 07/14/2000 |
| SEQ ID NO:8397 | 60/218,950 | 07/14/2000 |
| SEQ ID NO:8398 | 60/218,950 | 07/14/2000 |
| SEQ ID NO:8399 | 60/218,950 | 07/14/2000 |
| SEQ ID NO:8400 | 60/218,950 | 07/14/2000 |
| SEQ ID NO:8401 | 60/218,950 | 07/14/2000 |
| SEQ ID NO:8402 | 60/218,950 | 07/14/2000 |
| SEQ ID NO:8403 | 60/218,950 | 07/14/2000 |
| SEQ ID NO:8404 | 60/218,950 | 07/14/2000 |
| SEQ ID NO:8405 | 60/218,950 | 07/14/2000 |
| SEQ ID NO:8406 | 60/218,950 | 07/14/2000 |
| SEQ ID NO:8407 | 60/218,950 | 07/14/2000 |
| SEQ ID NO:8408 | 60/218,950 | 07/14/2000 |
| SEQ ID NO:8409 | 60/218,950 | 07/14/2000 |
| SEQ ID NO:8410 | 60/218,950 | 07/14/2000 |
| SEQ ID NO:8411 | 60/218,950 | 07/14/2000 |
| SEQ ID NO:8412 | 60/218,950 | 07/14/2000 |
| SEQ ID NO:8413 | 60/218,950 | 07/14/2000 |
| SEQ ID NO:8414 | 60/218,950 | 07/14/2000 |
| SEQ ID NO:8415 | 60/218,950 | 07/14/2000 |
| SEQ ID NO:8416 | 60/218,950 | 07/14/2000 |
| SEQ ID NO:8417 | 60/218,950 | 07/14/2000 |
| SEQ ID NO:8418 | 60/218,950 | 07/14/2000 |
| SEQ ID NO:8419 | 60/218,950 | 07/14/2000 |
| SEQ ID NO:8420 | 60/218,950 | 07/14/2000 |
| SEQ ID NO:8421 | 60/218,950 | 07/14/2000 |
| SEQ ID NO:8422 | 60/218,950 | 07/14/2000 |
| SEQ ID NO:8423 | 60/218,950 | 07/14/2000 |
| SEQ ID NO:8424 | 60/218,950 | 07/14/2000 |
| SEQ ID NO:8425 | 60/218,950 | 07/14/2000 |
| SEQ ID NO:8426 | 60/218,950 | 07/14/2000 |
| SEQ ID NO:8427 | 60/218,950 | 07/14/2000 |
| SEQ ID NO:8428 | 60/218,950 | 07/14/2000 |
| SEQ ID NO:8429 | 60/218,950 | 07/14/2000 |
| SEQ ID NO:8430 | 60/218,950 | 07/14/2000 |
| SEQ ID NO:8431 | 60/218,950 | 07/14/2000 |
| SEQ ID NO:8432 | 60/218,950 | 07/14/2000 |
| SEQ ID NO:8433 | 60/218,950 | 07/14/2000 |
| SEQ ID NO:8434 | 60/218,950 | 07/14/2000 |
| SEQ ID NO:8435 | 60/218,950 | 07/14/2000 |
| SEQ ID NO:8436 | 60/218,950 | 07/14/2000 |
| SEQ ID NO:8437 | 60/218,950 | 07/14/2000 |
| SEQ ID NO:8438 | 60/218,950 | 07/14/2000 |
| SEQ ID NO:8439 | 60/218,950 | 07/14/2000 |
| SEQ ID NO:8440 | 60/218,950 | 07/14/2000 |
| SEQ ID NO:8441 | 60/218,950 | 07/14/2000 |
| SEQ ID NO:8442 | 60/218,950 | 07/14/2000 |
| SEQ ID NO:8443 | 60/218,950 | 07/14/2000 |
| SEQ ID NO:8444 | 60/218,950 | 07/14/2000 |
| SEQ ID NO:8445 | 60/218,950 | 07/14/2000 |
| SEQ ID NO:8446 | 60/218,950 | 07/14/2000 |
| SEQ ID NO:8447 | 60/218,950 | 07/14/2000 |
| SEQ ID NO:8448 | 60/218,950 | 07/14/2000 |
| SEQ ID NO:8449 | 60/218,950 | 07/14/2000 |
| SEQ ID NO:8450 | 60/218,950 | 07/14/2000 |
| SEQ ID NO:8451 | 60/218,950 | 07/14/2000 |
| SEQ ID NO:8452 | 60/218,950 | 07/14/2000 |
| SEQ ID NO:8453 | 60/218,950 | 07/14/2000 |
| SEQ ID NO:8454 | 60/218,950 | 07/14/2000 |
| SEQ ID NO:8455 | 60/218,950 | 07/14/2000 |
| SEQ ID NO:8456 | 60/218,950 | 07/14/2000 |
| SEQ ID NO:8457 | 60/218,950 | 07/14/2000 |
| SEQ ID NO:8458 | 60/218,950 | 07/14/2000 |
| SEQ ID NO:8459 | 60/218,950 | 07/14/2000 |
| SEQ ID NO:8460 | 60/218,950 | 07/14/2000 |
| SEQ ID NO:8461 | 60/218,950 | 07/14/2000 |
| SEQ ID NO:8462 | 60/218,950 | 07/14/2000 |
| SEQ ID NO:8463 | 60/218,950 | 07/14/2000 |
| SEQ ID NO:8464 | 60/218,950 | 07/14/2000 |
| SEQ ID NO:8465 | 60/218,950 | 07/14/2000 |
| SEQ ID NO:8466 | 60/218,950 | 07/14/2000 |
| SEQ ID NO:8467 | 60/218,950 | 07/14/2000 |
| SEQ ID NO:8468 | 60/218,950 | 07/14/2000 |
| SEQ ID NO:8469 | 60/218,950 | 07/14/2000 |
| SEQ ID NO:8470 | 60/218,950 | 07/14/2000 |
| SEQ ID NO:8471 | 60/218,950 | 07/14/2000 |
| SEQ ID NO:8472 | 60/218,950 | 07/14/2000 |
| SEQ ID NO:8473 | 60/218,950 | 07/14/2000 |
| SEQ ID NO:8474 | 60/218,950 | 07/14/2000 |
| SEQ ID NO:8475 | 60/218,950 | 07/14/2000 |
| SEQ ID NO:8476 | 60/218,950 | 07/14/2000 |
| SEQ ID NO:8477 | 60/218,950 | 07/14/2000 |
| SEQ ID NO:8478 | 60/218,950 | 07/14/2000 |
| SEQ ID NO:8479 | 60/218,950 | 07/14/2000 |
| SEQ ID NO:8480 | 60/218,950 | 07/14/2000 |
| SEQ ID NO:8481 | 60/218,950 | 07/14/2000 |
| SEQ ID NO:8482 | 60/218,950 | 07/14/2000 |
| SEQ ID NO:8483 | 60/218,950 | 07/14/2000 |
| SEQ ID NO:8484 | 60/218,950 | 07/14/2000 |
| SEQ ID NO:8485 | 60/218,950 | 07/14/2000 |
| SEQ ID NO:8486 | 60/218,950 | 07/14/2000 |
| SEQ ID NO:8487 | 60/218,950 | 07/14/2000 |
| SEQ ID NO:8488 | 60/218,950 | 07/14/2000 |
| SEQ ID NO:8489 | 60/218,950 | 07/14/2000 |
| SEQ ID NO:8490 | 60/218,950 | 07/14/2000 |
| SEQ ID NO:8491 | 60/218,950 | 07/14/2000 |
| SEQ ID NO:8492 | 60/218,950 | 07/14/2000 |
| SEQ ID NO:8493 | 60/218,950 | 07/14/2000 |
| SEQ ID NO:8494 | 60/218,950 | 07/14/2000 |
| SEQ ID NO:8495 | 60/218,950 | 07/14/2000 |
| SEQ ID NO:8496 | 60/218,950 | 07/14/2000 |
| SEQ ID NO:8497 | 60/218,950 | 07/14/2000 |
| SEQ ID NO:8498 | 60/218,950 | 07/14/2000 |
| SEQ ID NO:8499 | 60/218,950 | 07/14/2000 |
| SEQ ID NO:8500 | 60/218,950 | 07/14/2000 |
| SEQ ID NO:8501 | 60/218,950 | 07/14/2000 |
| SEQ ID NO:8502 | 60/218,950 | 07/14/2000 |
| SEQ ID NO:8503 | 60/218,950 | 07/14/2000 |
| SEQ ID NO:8504 | 60/218,950 | 07/14/2000 |
| SEQ ID NO:8505 | 60/218,950 | 07/14/2000 |
| SEQ ID NO:8506 | 60/218,950 | 07/14/2000 |
| SEQ ID NO:8507 | 60/218,950 | 07/14/2000 |
| SEQ ID NO:8508 | 60/218,950 | 07/14/2000 |
| SEQ ID NO:8509 | 60/218,950 | 07/14/2000 |
| SEQ ID NO:8510 | 60/218,950 | 07/14/2000 |
| SEQ ID NO:8511 | 60/218,950 | 07/14/2000 |
| SEQ ID NO:8512 | 60/218,950 | 07/14/2000 |
| SEQ ID NO:8513 | 60/218,950 | 07/14/2000 |
| SEQ ID NO:8514 | 60/218,950 | 07/14/2000 |
| SEQ ID NO:8515 | 60/218,950 | 07/14/2000 |
| SEQ ID NO:8516 | 60/218,950 | 07/14/2000 |
| SEQ ID NO:8517 | 60/218,950 | 07/14/2000 |
| SEQ ID NO:8518 | 60/218,950 | 07/14/2000 |
| SEQ ID NO:8519 | 60/218,950 | 07/14/2000 |
| SEQ ID NO:8520 | 60/218,950 | 07/14/2000 |
| SEQ ID NO:8521 | 60/218,950 | 07/14/2000 |
| SEQ ID NO:8522 | 60/218,950 | 07/14/2000 |
| SEQ ID NO:8523 | 60/218,950 | 07/14/2000 |
| SEQ ID NO:8524 | 60/218,950 | 07/14/2000 |
| SEQ ID NO:8525 | 60/218,950 | 07/14/2000 |
| SEQ ID NO:8526 | 60/218,950 | 07/14/2000 |
| SEQ ID NO:8527 | 60/218,950 | 07/14/2000 |
| SEQ ID NO:8528 | 60/218,950 | 07/14/2000 |
| SEQ ID NO:8529 | 60/218,950 | 07/14/2000 |
| SEQ ID NO:8530 | 60/218,950 | 07/14/2000 |
| SEQ ID NO:8531 | 60/218,950 | 07/14/2000 |
| SEQ ID NO:8532 | 60/218,950 | 07/14/2000 |
| SEQ ID NO:8533 | 60/218,950 | 07/14/2000 |
| SEQ ID NO:8534 | 60/218,950 | 07/14/2000 |
| SEQ ID NO:8535 | 60/218,950 | 07/14/2000 |
| SEQ ID NO:8536 | 60/218,950 | 07/14/2000 |
| SEQ ID NO:8537 | 60/218,950 | 07/14/2000 |
| SEQ ID NO:8538 | 60/218,950 | 07/14/2000 |
| SEQ ID NO:8539 | 60/218,950 | 07/14/2000 |
| SEQ ID NO:8540 | 60/218,950 | 07/14/2000 |
| SEQ ID NO:8541 | 60/218,950 | 07/14/2000 |
| SEQ ID NO:8542 | 60/218,950 | 07/14/2000 |
| SEQ ID NO:8543 | 60/218,950 | 07/14/2000 |
| SEQ ID NO:8544 | 60/218,950 | 07/14/2000 |
| SEQ ID NO:8545 | 60/218,950 | 07/14/2000 |
| SEQ ID NO:8546 | 60/218,950 | 07/14/2000 |
| SEQ ID NO:8547 | 60/218,950 | 07/14/2000 |
| SEQ ID NO:8548 | 60/218,950 | 07/14/2000 |
| SEQ ID NO:8549 | 60/218,950 | 07/14/2000 |
| SEQ ID NO:8550 | 60/218,950 | 07/14/2000 |
| SEQ ID NO:8551 | 60/218,950 | 07/14/2000 |
| SEQ ID NO:8552 | 60/218,950 | 07/14/2000 |
| SEQ ID NO:8553 | 60/218,950 | 07/14/2000 |
| SEQ ID NO:8554 | 60/218,950 | 07/14/2000 |
| SEQ ID NO:8555 | 60/218,950 | 07/14/2000 |
| SEQ ID NO:8556 | 60/218,950 | 07/14/2000 |
| SEQ ID NO:8557 | 60/218,950 | 07/14/2000 |
| SEQ ID NO:8558 | 60/218,950 | 07/14/2000 |
| SEQ ID NO:8559 | 60/218,950 | 07/14/2000 |
| SEQ ID NO:8560 | 60/218,950 | 07/14/2000 |
| SEQ ID NO:8561 | 60/218,950 | 07/14/2000 |
| SEQ ID NO:8562 | 60/218,950 | 07/14/2000 |
| SEQ ID NO:8563 | 60/218,950 | 07/14/2000 |
| SEQ ID NO:8564 | 60/218,950 | 07/14/2000 |
| SEQ ID NO:8565 | 60/218,950 | 07/14/2000 |
| SEQ ID NO:8566 | 60/218,950 | 07/14/2000 |
| SEQ ID NO:8567 | 60/218,950 | 07/14/2000 |
| SEQ ID NO:8568 | 60/218,950 | 07/14/2000 |
| SEQ ID NO:8569 | 60/218,950 | 07/14/2000 |
| SEQ ID NO:8570 | 60/218,950 | 07/14/2000 |
| SEQ ID NO:8571 | 60/218,950 | 07/14/2000 |
| SEQ ID NO:8572 | 60/218,950 | 07/14/2000 |
| SEQ ID NO:8573 | 60/218,950 | 07/14/2000 |
| SEQ ID NO:8574 | 60/218,950 | 07/14/2000 |
| SEQ ID NO:8575 | 60/218,950 | 07/14/2000 |
| SEQ ID NO:8576 | 60/218,950 | 07/14/2000 |
| SEQ ID NO:8577 | 60/218,950 | 07/14/2000 |
| SEQ ID NO:8578 | 60/218,950 | 07/14/2000 |
| SEQ ID NO:8579 | 60/218,950 | 07/14/2000 |
| SEQ ID NO:8580 | 60/218,950 | 07/14/2000 |
| SEQ ID NO:8581 | 60/218,950 | 07/14/2000 |
| SEQ ID NO:8582 | 60/218,950 | 07/14/2000 |
| SEQ ID NO:8583 | 60/218,950 | 07/14/2000 |
| SEQ ID NO:8584 | 60/218,950 | 07/14/2000 |
| SEQ ID NO:8585 | 60/218,950 | 07/14/2000 |
| SEQ ID NO:8586 | 60/218,950 | 07/14/2000 |
| SEQ ID NO:8587 | 60/218,950 | 07/14/2000 |
| SEQ ID NO:8588 | 60/218,950 | 07/14/2000 |
| SEQ ID NO:8589 | 60/218,950 | 07/14/2000 |
| SEQ ID NO:8590 | 60/218,950 | 07/14/2000 |
| SEQ ID NO:8591 | 60/218,950 | 07/14/2000 |
| SEQ ID NO:8592 | 60/218,950 | 07/14/2000 |
| SEQ ID NO:8593 | 60/218,950 | 07/14/2000 |
| SEQ ID NO:8594 | 60/218,950 | 07/14/2000 |
| SEQ ID NO:8595 | 60/218,950 | 07/14/2000 |
| SEQ ID NO:8596 | 60/218,950 | 07/14/2000 |
| SEQ ID NO:8597 | 60/218,950 | 07/14/2000 |
| SEQ ID NO:8598 | 60/218,950 | 07/14/2000 |
| SEQ ID NO:8599 | 60/218,950 | 07/14/2000 |
| SEQ ID NO:8600 | 60/218,950 | 07/14/2000 |
| SEQ ID NO:8601 | 60/218,950 | 07/14/2000 |
| SEQ ID NO:8602 | 60/218,950 | 07/14/2000 |
| SEQ ID NO:8603 | 60/218,950 | 07/14/2000 |
| SEQ ID NO:8604 | 60/218,950 | 07/14/2000 |
| SEQ ID NO:8605 | 60/218,950 | 07/14/2000 |
| SEQ ID NO:8606 | 60/218,950 | 07/14/2000 |
| SEQ ID NO:8607 | 60/218,950 | 07/14/2000 |
| SEQ ID NO:8608 | 60/218,950 | 07/14/2000 |
| SEQ ID NO:8609 | 60/218,950 | 07/14/2000 |
| SEQ ID NO:8610 | 60/218,950 | 07/14/2000 |
| SEQ ID NO:8611 | 60/218,950 | 07/14/2000 |
| SEQ ID NO:8612 | 60/218,950 | 07/14/2000 |
| SEQ ID NO:8613 | 60/218,950 | 07/14/2000 |
| SEQ ID NO:8614 | 60/218,950 | 07/14/2000 |
| SEQ ID NO:8615 | 60/218,950 | 07/14/2000 |
| SEQ ID NO:8616 | 60/218,950 | 07/14/2000 |
| SEQ ID NO:8617 | 60/218,950 | 07/14/2000 |
| SEQ ID NO:8618 | 60/218,950 | 07/14/2000 |
| SEQ ID NO:8619 | 60/218,950 | 07/14/2000 |
| SEQ ID NO:8620 | 60/218,950 | 07/14/2000 |
| SEQ ID NO:8621 | 60/218,950 | 07/14/2000 |
| SEQ ID NO:8622 | 60/218,950 | 07/14/2000 |
| SEQ ID NO:8623 | 60/218,950 | 07/14/2000 |
| SEQ ID NO:8624 | 60/218,950 | 07/14/2000 |
| SEQ ID NO:8625 | 60/218,950 | 07/14/2000 |
| SEQ ID NO:8626 | 60/218,950 | 07/14/2000 |
| SEQ ID NO:8627 | 60/218,950 | 07/14/2000 |
| SEQ ID NO:8628 | 60/218,950 | 07/14/2000 |
| SEQ ID NO:8629 | 60/218,950 | 07/14/2000 |
| SEQ ID NO:8630 | 60/218,950 | 07/14/2000 |
| SEQ ID NO:8631 | 60/218,950 | 07/14/2000 |
| SEQ ID NO:8632 | 60/218,950 | 07/14/2000 |
| SEQ ID NO:8633 | 60/218,950 | 07/14/2000 |
| SEQ ID NO:8634 | 60/218,950 | 07/14/2000 |
| SEQ ID NO:8635 | 60/218,950 | 07/14/2000 |
| SEQ ID NO:8636 | 60/218,950 | 07/14/2000 |
| SEQ ID NO:8637 | 60/218,950 | 07/14/2000 |
| SEQ ID NO:8638 | 60/218,950 | 07/14/2000 |
| SEQ ID NO:8639 | 60/218,950 | 07/14/2000 |
| SEQ ID NO:8640 | 60/218,950 | 07/14/2000 |
| SEQ ID NO:8641 | 60/218,950 | 07/14/2000 |
| SEQ ID NO:8642 | 60/218,950 | 07/14/2000 |
| SEQ ID NO:8643 | 60/218,950 | 07/14/2000 |
| SEQ ID NO:8644 | 60/218,950 | 07/14/2000 |
| SEQ ID NO:8645 | 60/218,950 | 07/14/2000 |
| SEQ ID NO:8646 | 60/218,950 | 07/14/2000 |
| SEQ ID NO:8647 | 60/218,950 | 07/14/2000 |
| SEQ ID NO:8648 | 60/218,950 | 07/14/2000 |
| SEQ ID NO:8649 | 60/218,950 | 07/14/2000 |
| SEQ ID NO:8650 | 60/218,950 | 07/14/2000 |
| SEQ ID NO:8651 | 60/218,950 | 07/14/2000 |
| SEQ ID NO:8652 | 60/218,950 | 07/14/2000 |
| SEQ ID NO:8653 | 60/218,950 | 07/14/2000 |
| SEQ ID NO:8654 | 60/218,950 | 07/14/2000 |
| SEQ ID NO:8655 | 60/218,950 | 07/14/2000 |
| SEQ ID NO:8656 | 60/218,950 | 07/14/2000 |
| SEQ ID NO:8657 | 60/218,950 | 07/14/2000 |
| SEQ ID NO:8658 | 60/218,950 | 07/14/2000 |
| SEQ ID NO:8659 | 60/218,950 | 07/14/2000 |
| SEQ ID NO:8660 | 60/218,950 | 07/14/2000 |
| SEQ ID NO:8661 | 60/218,950 | 07/14/2000 |
| SEQ ID NO:8662 | 60/218,950 | 07/14/2000 |
| SEQ ID NO:8663 | 60/218,950 | 07/14/2000 |
| SEQ ID NO:8664 | 60/218,950 | 07/14/2000 |
| SEQ ID NO:8665 | 60/218,950 | 07/14/2000 |
| SEQ ID NO:8666 | 60/218,950 | 07/14/2000 |
| SEQ ID NO:8667 | 60/218,950 | 07/14/2000 |
| SEQ ID NO:8668 | 60/218,950 | 07/14/2000 |
| SEQ ID NO:8669 | 60/218,950 | 07/14/2000 |
| SEQ ID NO:8670 | 60/218,950 | 07/14/2000 |
| SEQ ID NO:8671 | 60/218,950 | 07/14/2000 |
| SEQ ID NO:8672 | 60/218,950 | 07/14/2000 |
| SEQ ID NO:8673 | 60/218,950 | 07/14/2000 |
| SEQ ID NO:8674 | 60/218,950 | 07/14/2000 |
| SEQ ID NO:8675 | 60/218,950 | 07/14/2000 |
| SEQ ID NO:8676 | 60/218,950 | 07/14/2000 |
| SEQ ID NO:8677 | 60/218,950 | 07/14/2000 |
| SEQ ID NO:8678 | 60/218,950 | 07/14/2000 |
| SEQ ID NO:8679 | 60/218,950 | 07/14/2000 |
| SEQ ID NO:8680 | 60/218,950 | 07/14/2000 |
| SEQ ID NO:8681 | 60/218,950 | 07/14/2000 |
| SEQ ID NO:8682 | 60/218,950 | 07/14/2000 |
| SEQ ID NO:8683 | 60/218,950 | 07/14/2000 |
| SEQ ID NO:8684 | 60/218,950 | 07/14/2000 |
| SEQ ID NO:8685 | 60/218,950 | 07/14/2000 |
| SEQ ID NO:8686 | 60/218,950 | 07/14/2000 |
| SEQ ID NO:8687 | 60/218,950 | 07/14/2000 |
| SEQ ID NO:8688 | 60/218,950 | 07/14/2000 |
| SEQ ID NO:8689 | 60/218,950 | 07/14/2000 |
| SEQ ID NO:8690 | 60/218,950 | 07/14/2000 |
| SEQ ID NO:8691 | 60/218,950 | 07/14/2000 |
| SEQ ID NO:8692 | 60/218,950 | 07/14/2000 |
| SEQ ID NO:8693 | 60/218,950 | 07/14/2000 |
| SEQ ID NO:8694 | 60/218,950 | 07/14/2000 |
| SEQ ID NO:8695 | 60/218,950 | 07/14/2000 |
| SEQ ID NO:8696 | 60/218,950 | 07/14/2000 |
| SEQ ID NO:8697 | 60/218,950 | 07/14/2000 |
| SEQ ID NO:8698 | 60/218,950 | 07/14/2000 |
| SEQ ID NO:8699 | 60/218,950 | 07/14/2000 |
| SEQ ID NO:8700 | 60/218,950 | 07/14/2000 |
| SEQ ID NO:8701 | 60/218,950 | 07/14/2000 |
| SEQ ID NO:8702 | 60/218,950 | 07/14/2000 |
| SEQ ID NO:8703 | 60/218,950 | 07/14/2000 |
| SEQ ID NO:8704 | 60/218,950 | 07/14/2000 |
| SEQ ID NO:8705 | 60/218,950 | 07/14/2000 |
| SEQ ID NO:8706 | 60/218,950 | 07/14/2000 |
| SEQ ID NO:8707 | 60/218,950 | 07/14/2000 |
| SEQ ID NO:8708 | 60/218,950 | 07/14/2000 |
| SEQ ID NO:8709 | 60/218,950 | 07/14/2000 |
| SEQ ID NO:8710 | 60/218,950 | 07/14/2000 |
| SEQ ID NO:8711 | 60/218,950 | 07/14/2000 |
| SEQ ID NO:8712 | 60/218,950 | 07/14/2000 |
| SEQ ID NO:8713 | 60/218,950 | 07/14/2000 |
| SEQ ID NO:8714 | 60/218,950 | 07/14/2000 |
| SEQ ID NO:8715 | 60/218,950 | 07/14/2000 |
| SEQ ID NO:8716 | 60/218,950 | 07/14/2000 |
| SEQ ID NO:8717 | 60/218,950 | 07/14/2000 |
| SEQ ID NO:8718 | 60/218,950 | 07/14/2000 |
| SEQ ID NO:8719 | 60/218,950 | 07/14/2000 |
| SEQ ID NO:8720 | 60/218,950 | 07/14/2000 |
| SEQ ID NO:8721 | 60/218,950 | 07/14/2000 |
| SEQ ID NO:8722 | 60/218,950 | 07/14/2000 |
| SEQ ID NO:8723 | 60/218,950 | 07/14/2000 |
| SEQ ID NO:8724 | 60/218,950 | 07/14/2000 |
| SEQ ID NO:8725 | 60/218,950 | 07/14/2000 |
| SEQ ID NO:8726 | 60/218,950 | 07/14/2000 |
| SEQ ID NO:8727 | 60/218,950 | 07/14/2000 |
| SEQ ID NO:8728 | 60/218,950 | 07/14/2000 |
| SEQ ID NO:8729 | 60/218,950 | 07/14/2000 |
| SEQ ID NO:8730 | 60/218,950 | 07/14/2000 |
| SEQ ID NO:8731 | 60/218,950 | 07/14/2000 |
| SEQ ID NO:8732 | 60/218,950 | 07/14/2000 |
| SEQ ID NO:8733 | 60/218,950 | 07/14/2000 |
| SEQ ID NO:8734 | 60/218,950 | 07/14/2000 |
| SEQ ID NO:8735 | 60/218,950 | 07/14/2000 |
| SEQ ID NO:8736 | 60/218,950 | 07/14/2000 |
| SEQ ID NO:8737 | 60/218,950 | 07/14/2000 |
| SEQ ID NO:8738 | 60/218,950 | 07/14/2000 |
| SEQ ID NO:8739 | 60/218,950 | 07/14/2000 |
| SEQ ID NO:8740 | 60/218,950 | 07/14/2000 |
| SEQ ID NO:8741 | 60/218,950 | 07/14/2000 |
| SEQ ID NO:8742 | 60/218,950 | 07/14/2000 |
| SEQ ID NO:8743 | 60/218,950 | 07/14/2000 |
| SEQ ID NO:8744 | 60/218,950 | 07/14/2000 |
| SEQ ID NO:8745 | 60/218,950 | 07/14/2000 |
| SEQ ID NO:8746 | 60/218,950 | 07/14/2000 |
| SEQ ID NO:8747 | 60/218,950 | 07/14/2000 |
| SEQ ID NO:8748 | 60/218,950 | 07/14/2000 |
| SEQ ID NO:8749 | 60/218,950 | 07/14/2000 |
| SEQ ID NO:8750 | 60/218,950 | 07/14/2000 |
| SEQ ID NO:8751 | 60/218,950 | 07/14/2000 |
| SEQ ID NO:8752 | 60/218,950 | 07/14/2000 |
| SEQ ID NO:8753 | 60/218,950 | 07/14/2000 |
| SEQ ID NO:8754 | 60/218,950 | 07/14/2000 |
| SEQ ID NO:8755 | 60/218,950 | 07/14/2000 |
| SEQ ID NO:8756 | 60/218,950 | 07/14/2000 |
| SEQ ID NO:8757 | 60/218,950 | 07/14/2000 |
| SEQ ID NO:8758 | 60/218,950 | 07/14/2000 |
| SEQ ID NO:8759 | 60/218,950 | 07/14/2000 |
| SEQ ID NO:8760 | 60/218,950 | 07/14/2000 |
| SEQ ID NO:8761 | 60/218,950 | 07/14/2000 |
| SEQ ID NO:8762 | 60/218,950 | 07/14/2000 |
| SEQ ID NO:8763 | 60/218,950 | 07/14/2000 |
| SEQ ID NO:8764 | 60/218,950 | 07/14/2000 |
| SEQ ID NO:8765 | 60/218,950 | 07/14/2000 |
| SEQ ID NO:8766 | 60/218,950 | 07/14/2000 |
| SEQ ID NO:8767 | 60/218,950 | 07/14/2000 |
| SEQ ID NO:8768 | 60/218,950 | 07/14/2000 |
| SEQ ID NO:8769 | 60/218,950 | 07/14/2000 |
| SEQ ID NO:8770 | 60/218,950 | 07/14/2000 |
| SEQ ID NO:8771 | 60/218,950 | 07/14/2000 |
| SEQ ID NO:8772 | 60/218,950 | 07/14/2000 |
| SEQ ID NO:8773 | 60/218,950 | 07/14/2000 |
| SEQ ID NO:8774 | 60/218,950 | 07/14/2000 |
| SEQ ID NO:8775 | 60/218,950 | 07/14/2000 |
| SEQ ID NO:8776 | 60/218,950 | 07/14/2000 |
| SEQ ID NO:8777 | 60/218,950 | 07/14/2000 |
| SEQ ID NO:8778 | 60/218,950 | 07/14/2000 |
| SEQ ID NO:8779 | 60/218,950 | 07/14/2000 |
| SEQ ID NO:8780 | 60/218,950 | 07/14/2000 |
| SEQ ID NO:8781 | 60/218,950 | 07/14/2000 |
| SEQ ID NO:8782 | 60/218,950 | 07/14/2000 |
| SEQ ID NO:8783 | 60/218,950 | 07/14/2000 |
| SEQ ID NO:8784 | 60/218,950 | 07/14/2000 |
| SEQ ID NO:8785 | 60/218,950 | 07/14/2000 |
| SEQ ID NO:8786 | 60/218,950 | 07/14/2000 |
| SEQ ID NO:8787 | 60/218,950 | 07/14/2000 |
| SEQ ID NO:8788 | 60/218,950 | 07/14/2000 |
| SEQ ID NO:8789 | 60/218,950 | 07/14/2000 |
| SEQ ID NO:8790 | 60/218,950 | 07/14/2000 |
| SEQ ID NO:8791 | 60/218,950 | 07/14/2000 |
| SEQ ID NO:8792 | 60/218,950 | 07/14/2000 |
| SEQ ID NO:8793 | 60/218,950 | 07/14/2000 |
| SEQ ID NO:8794 | 60/218,950 | 07/14/2000 |
| SEQ ID NO:8795 | 60/218,950 | 07/14/2000 |
| SEQ ID NO:8796 | 60/218,950 | 07/14/2000 |
| SEQ ID NO:8797 | 60/218,950 | 07/14/2000 |
| SEQ ID NO:8798 | 60/218,950 | 07/14/2000 |
| SEQ ID NO:8799 | 60/218,950 | 07/14/2000 |
| SEQ ID NO:8800 | 60/218,950 | 07/14/2000 |
| SEQ ID NO:8801 | 60/218,950 | 07/14/2000 |
| SEQ ID NO:8802 | 60/218,950 | 07/14/2000 |
| SEQ ID NO:8803 | 60/218,950 | 07/14/2000 |
| SEQ ID NO:8804 | 60/218,950 | 07/14/2000 |
| SEQ ID NO:8805 | 60/218,950 | 07/14/2000 |
| SEQ ID NO:8806 | 60/218,950 | 07/14/2000 |
| SEQ ID NO:8807 | 60/218,950 | 07/14/2000 |
| SEQ ID NO:8808 | 60/218,950 | 07/14/2000 |
| SEQ ID NO:8809 | 60/218,950 | 07/14/2000 |
| SEQ ID NO:8810 | 60/218,950 | 07/14/2000 |
| SEQ ID NO:8811 | 60/218,950 | 07/14/2000 |
| SEQ ID NO:8812 | 60/218,950 | 07/14/2000 |
| SEQ ID NO:8813 | 60/218,950 | 07/14/2000 |
| SEQ ID NO:8814 | 60/218,950 | 07/14/2000 |
| SEQ ID NO:8815 | 60/218,950 | 07/14/2000 |
| SEQ ID NO:8816 | 60/218,950 | 07/14/2000 |
| SEQ ID NO:8817 | 60/218,950 | 07/14/2000 |
| SEQ ID NO:8818 | 60/218,950 | 07/14/2000 |
| SEQ ID NO:8819 | 60/218,950 | 07/14/2000 |
| SEQ ID NO:8820 | 60/218,950 | 07/14/2000 |
| SEQ ID NO:8821 | 60/218,950 | 07/14/2000 |
| SEQ ID NO:8822 | 60/218,950 | 07/14/2000 |
| SEQ ID NO:8823 | 60/218,950 | 07/14/2000 |
| SEQ ID NO:8824 | 60/218,950 | 07/14/2000 |
| SEQ ID NO:8825 | 60/218,950 | 07/14/2000 |
| SEQ ID NO:8826 | 60/218,950 | 07/14/2000 |
| SEQ ID NO:8827 | 60/218,950 | 07/14/2000 |
| SEQ ID NO:8828 | 60/218,950 | 07/14/2000 |
| SEQ ID NO:8829 | 60/218,950 | 07/14/2000 |
| SEQ ID NO:8830 | 60/218,950 | 07/14/2000 |
| SEQ ID NO:8831 | 60/218,950 | 07/14/2000 |
| SEQ ID NO:8832 | 60/218,950 | 07/14/2000 |
| SEQ ID NO:8833 | 60/218,950 | 07/14/2000 |
| SEQ ID NO:8834 | 60/218,950 | 07/14/2000 |
| SEQ ID NO:8835 | 60/218,950 | 07/14/2000 |
| SEQ ID NO:8836 | 60/218,950 | 07/14/2000 |
| SEQ ID NO:8837 | 60/218,950 | 07/14/2000 |
| SEQ ID NO:8838 | 60/218,950 | 07/14/2000 |
| SEQ ID NO:8839 | 60/218,950 | 07/14/2000 |
| SEQ ID NO:8840 | 60/218,950 | 07/14/2000 |
| SEQ ID NO:8841 | 60/218,950 | 07/14/2000 |
| SEQ ID NO:8842 | 60/218,950 | 07/14/2000 |
| SEQ ID NO:8843 | 60/218,950 | 07/14/2000 |
| SEQ ID NO:8844 | 60/218,950 | 07/14/2000 |
| SEQ ID NO:8845 | 60/218,950 | 07/14/2000 |
| SEQ ID NO:8846 | 60/218,950 | 07/14/2000 |
| SEQ ID NO:8847 | 60/218,950 | 07/14/2000 |
| SEQ ID NO:8848 | 60/218,950 | 07/14/2000 |
| SEQ ID NO:8849 | 60/218,950 | 07/14/2000 |
| SEQ ID NO:8850 | 60/218,950 | 07/14/2000 |
| SEQ ID NO:8851 | 60/218,950 | 07/14/2000 |
| SEQ ID NO:8852 | 60/218,950 | 07/14/2000 |
| SEQ ID NO:8853 | 60/218,950 | 07/14/2000 |
| SEQ ID NO:8854 | 60/218,950 | 07/14/2000 |
| SEQ ID NO:8855 | 60/218,950 | 07/14/2000 |
| SEQ ID NO:8856 | 60/218,950 | 07/14/2000 |
| SEQ ID NO:8857 | 60/218,950 | 07/14/2000 |
| SEQ ID NO:8858 | 60/218,950 | 07/14/2000 |
| SEQ ID NO:8859 | 60/218,950 | 07/14/2000 |
| SEQ ID NO:8860 | 60/218,950 | 07/14/2000 |
| SEQ ID NO:8861 | 60/218,950 | 07/14/2000 |
| SEQ ID NO:8862 | 60/218,950 | 07/14/2000 |
| SEQ ID NO:8863 | 60/218,950 | 07/14/2000 |
| SEQ ID NO:8864 | 60/218,950 | 07/14/2000 |
| SEQ ID NO:8865 | 60/218,950 | 07/14/2000 |
| SEQ ID NO:8866 | 60/218,950 | 07/14/2000 |
| SEQ ID NO:8867 | 60/218,950 | 07/14/2000 |
| SEQ ID NO:8868 | 60/218,950 | 07/14/2000 |
| SEQ ID NO:8869 | 60/218,950 | 07/14/2000 |
| SEQ ID NO:8870 | 60/218,950 | 07/14/2000 |
| SEQ ID NO:8871 | 60/218,950 | 07/14/2000 |
| SEQ ID NO:8872 | 60/218,950 | 07/14/2000 |
| SEQ ID NO:8873 | 60/218,950 | 07/14/2000 |
| SEQ ID NO:8874 | 60/218,950 | 07/14/2000 |
| SEQ ID NO:8875 | 60/218,950 | 07/14/2000 |
| SEQ ID NO:8876 | 60/218,950 | 07/14/2000 |
| SEQ ID NO:8877 | 60/218,950 | 07/14/2000 |
| SEQ ID NO:8878 | 60/218,950 | 07/14/2000 |
| SEQ ID NO:8879 | 60/218,950 | 07/14/2000 |
| SEQ ID NO:8880 | 60/218,950 | 07/14/2000 |
| SEQ ID NO:8881 | 60/218,950 | 07/14/2000 |
| SEQ ID NO:8882 | 60/218,950 | 07/14/2000 |
| SEQ ID NO:8883 | 60/218,950 | 07/14/2000 |
| SEQ ID NO:8884 | 60/218,950 | 07/14/2000 |
| SEQ ID NO:8885 | 60/218,950 | 07/14/2000 |
| SEQ ID NO:8886 | 60/218,950 | 07/14/2000 |
| SEQ ID NO:8887 | 60/218,950 | 07/14/2000 |
| SEQ ID NO:8888 | 60/218,950 | 07/14/2000 |
| SEQ ID NO:8889 | 60/218,950 | 07/14/2000 |
| SEQ ID NO:8890 | 60/218,950 | 07/14/2000 |
| SEQ ID NO:8891 | 60/218,950 | 07/14/2000 |
| SEQ ID NO:8892 | 60/218,950 | 07/14/2000 |
| SEQ ID NO:8893 | 60/218,950 | 07/14/2000 |
| SEQ ID NO:8894 | 60/218,950 | 07/14/2000 |
| SEQ ID NO:8895 | 60/218,950 | 07/14/2000 |
| SEQ ID NO:8896 | 60/218,950 | 07/14/2000 |
| SEQ ID NO:8897 | 60/218,950 | 07/14/2000 |
| SEQ ID NO:8898 | 60/218,950 | 07/14/2000 |
| SEQ ID NO:8899 | 60/218,950 | 07/14/2000 |
| SEQ ID NO:8900 | 60/218,950 | 07/14/2000 |
| SEQ ID NO:8901 | 60/218,950 | 07/14/2000 |
| SEQ ID NO:8902 | 60/218,950 | 07/14/2000 |
| SEQ ID NO:8903 | 60/218,950 | 07/14/2000 |
| SEQ ID NO:8904 | 60/218,950 | 07/14/2000 |
| SEQ ID NO:8905 | 60/218,950 | 07/14/2000 |
| SEQ ID NO:8906 | 60/218,950 | 07/14/2000 |
| SEQ ID NO:8907 | 60/218,950 | 07/14/2000 |
| SEQ ID NO:8908 | 60/218,950 | 07/14/2000 |
| SEQ ID NO:8909 | 60/218,950 | 07/14/2000 |
| SEQ ID NO:8910 | 60/218,950 | 07/14/2000 |
| SEQ ID NO:8911 | 60/218,950 | 07/14/2000 |
| SEQ ID NO:8912 | 60/218,950 | 07/14/2000 |
| SEQ ID NO:8913 | 60/218,950 | 07/14/2000 |
| SEQ ID NO:8914 | 60/218,950 | 07/14/2000 |
| SEQ ID NO:8915 | 60/218,950 | 07/14/2000 |
| SEQ ID NO:8916 | 60/218,950 | 07/14/2000 |
| SEQ ID NO:8917 | 60/218,950 | 07/14/2000 |
| SEQ ID NO:8918 | 60/218,950 | 07/14/2000 |
| SEQ ID NO:8919 | 60/218,950 | 07/14/2000 |
| SEQ ID NO:8920 | 60/218,950 | 07/14/2000 |
| SEQ ID NO:8921 | 60/218,950 | 07/14/2000 |
| SEQ ID NO:8922 | 60/218,950 | 07/14/2000 |
| SEQ ID NO:8923 | 60/218,950 | 07/14/2000 |
| SEQ ID NO:8924 | 60/218,950 | 07/14/2000 |
| SEQ ID NO:8925 | 60/218,950 | 07/14/2000 |
| SEQ ID NO:8926 | 60/218,950 | 07/14/2000 |
| SEQ ID NO:8927 | 60/218,950 | 07/14/2000 |
| SEQ ID NO:8928 | 60/218,950 | 07/14/2000 |
| SEQ ID NO:8929 | 60/218,950 | 07/14/2000 |
| SEQ ID NO:8930 | 60/218,950 | 07/14/2000 |
| SEQ ID NO:8931 | 60/218,950 | 07/14/2000 |
| SEQ ID NO:8932 | 60/218,950 | 07/14/2000 |
| SEQ ID NO:8933 | 60/218,950 | 07/14/2000 |
| SEQ ID NO:8934 | 60/218,950 | 07/14/2000 |
| SEQ ID NO:8935 | 60/218,950 | 07/14/2000 |
| SEQ ID NO:8936 | 60/218,950 | 07/14/2000 |
| SEQ ID NO:8937 | 60/218,950 | 07/14/2000 |
| SEQ ID NO:8938 | 60/218,950 | 07/14/2000 |
| SEQ ID NO:8939 | 60/218,950 | 07/14/2000 |
| SEQ ID NO:8940 | 60/218,950 | 07/14/2000 |
| SEQ ID NO:8941 | 60/218,950 | 07/14/2000 |
| SEQ ID NO:8942 | 60/218,950 | 07/14/2000 |
| SEQ ID NO:8943 | 60/218,950 | 07/14/2000 |
| SEQ ID NO:8944 | 60/218,950 | 07/14/2000 |
| SEQ ID NO:8945 | 60/218,950 | 07/14/2000 |
| SEQ ID NO:8946 | 60/218,950 | 07/14/2000 |
| SEQ ID NO:8947 | 60/218,950 | 07/14/2000 |
| SEQ ID NO:8948 | 60/218,950 | 07/14/2000 |
| SEQ ID NO:8949 | 60/218,950 | 07/14/2000 |
| SEQ ID NO:8950 | 60/218,950 | 07/14/2000 |
| SEQ ID NO:8951 | 60/218,950 | 07/14/2000 |
| SEQ ID NO:8952 | 60/218,950 | 07/14/2000 |
| SEQ ID NO:8953 | 60/218,950 | 07/14/2000 |
| SEQ ID NO:8954 | 60/218,950 | 07/14/2000 |
| SEQ ID NO:8955 | 60/218,950 | 07/14/2000 |
| SEQ ID NO:8956 | 60/218,950 | 07/14/2000 |
| SEQ ID NO:8957 | 60/218,950 | 07/14/2000 |
| SEQ ID NO:8958 | 60/218,950 | 07/14/2000 |
| SEQ ID NO:8959 | 60/218,950 | 07/14/2000 |
| SEQ ID NO:8960 | 60/218,950 | 07/14/2000 |
| SEQ ID NO:8961 | 60/218,950 | 07/14/2000 |
| SEQ ID NO:8962 | 60/218,950 | 07/14/2000 |
| SEQ ID NO:8963 | 60/218,950 | 07/14/2000 |
| SEQ ID NO:8964 | 60/218,950 | 07/14/2000 |
| SEQ ID NO:8965 | 60/218,950 | 07/14/2000 |
| SEQ ID NO:8966 | 60/218,950 | 07/14/2000 |
| SEQ ID NO:8967 | 60/218,950 | 07/14/2000 |
| SEQ ID NO:8968 | 60/218,950 | 07/14/2000 |
| SEQ ID NO:8969 | 60/218,950 | 07/14/2000 |
| SEQ ID NO:8970 | 60/218,950 | 07/14/2000 |
| SEQ ID NO:8971 | 60/218,950 | 07/14/2000 |
| SEQ ID NO:8972 | 60/218,950 | 07/14/2000 |
| SEQ ID NO:8973 | 60/218,950 | 07/14/2000 |
| SEQ ID NO:8974 | 60/218,950 | 07/14/2000 |
| SEQ ID NO:8975 | 60/218,950 | 07/14/2000 |
| SEQ ID NO:8976 | 60/218,950 | 07/14/2000 |
| SEQ ID NO:8977 | 60/218,950 | 07/14/2000 |
| SEQ ID NO:8978 | 60/218,950 | 07/14/2000 |
| SEQ ID NO:8979 | 60/218,950 | 07/14/2000 |
| SEQ ID NO:8980 | 60/218,950 | 07/14/2000 |
| SEQ ID NO:8981 | 60/218,950 | 07/14/2000 |
| SEQ ID NO:8982 | 60/218,950 | 07/14/2000 |
| SEQ ID NO:8983 | 60/218,950 | 07/14/2000 |
| SEQ ID NO:8984 | 60/218,950 | 07/14/2000 |
| SEQ ID NO:8985 | 60/218,950 | 07/14/2000 |
| SEQ ID NO:8986 | 60/218,950 | 07/14/2000 |
| SEQ ID NO:8987 | 60/218,950 | 07/14/2000 |
| SEQ ID NO:8988 | 60/218,950 | 07/14/2000 |
| SEQ ID NO:8989 | 60/218,950 | 07/14/2000 |
| SEQ ID NO:8990 | 60/218,950 | 07/14/2000 |
| SEQ ID NO:8991 | 60/218,950 | 07/14/2000 |
| SEQ ID NO:8992 | 60/218,950 | 07/14/2000 |
| SEQ ID NO:8993 | 60/218,950 | 07/14/2000 |
| SEQ ID NO:8994 | 60/218,950 | 07/14/2000 |
| SEQ ID NO:8995 | 60/218,950 | 07/14/2000 |
| SEQ ID NO:8996 | 60/218,950 | 07/14/2000 |
| SEQ ID NO:8997 | 60/218,950 | 07/14/2000 |
| SEQ ID NO:8998 | 60/218,950 | 07/14/2000 |
| SEQ ID NO:8999 | 60/218,950 | 07/14/2000 |
| SEQ ID NO:9000 | 60/218,950 | 07/14/2000 |
| SEQ ID NO:9001 | 60/218,950 | 07/14/2000 |
| SEQ ID NO:9002 | 60/218,950 | 07/14/2000 |
| SEQ ID NO:9003 | 60/218,950 | 07/14/2000 |
| SEQ ID NO:9004 | 60/218,950 | 07/14/2000 |
| SEQ ID NO:9005 | 60/218,950 | 07/14/2000 |
| SEQ ID NO:9006 | 60/218,950 | 07/14/2000 |
| SEQ ID NO:9007 | 60/218,950 | 07/14/2000 |
| SEQ ID NO:9008 | 60/218,950 | 07/14/2000 |
| SEQ ID NO:9009 | 60/218,950 | 07/14/2000 |
| SEQ ID NO:9010 | 60/218,950 | 07/14/2000 |
| SEQ ID NO:9011 | 60/218,950 | 07/14/2000 |
| SEQ ID NO:9012 | 60/218,950 | 07/14/2000 |
| SEQ ID NO:9013 | 60/218,950 | 07/14/2000 |
| SEQ ID NO:9014 | 60/218,950 | 07/14/2000 |
| SEQ ID NO:9015 | 60/218,950 | 07/14/2000 |
| SEQ ID NO:9016 | 60/218,950 | 07/14/2000 |
| SEQ ID NO:9017 | 60/218,950 | 07/14/2000 |
| SEQ ID NO:9018 | 60/218,950 | 07/14/2000 |
| SEQ ID NO:9019 | 60/218,950 | 07/14/2000 |
| SEQ ID NO:9020 | 60/218,950 | 07/14/2000 |
| SEQ ID NO:9021 | 60/218,950 | 07/14/2000 |
| SEQ ID NO:9022 | 60/218,950 | 07/14/2000 |
| SEQ ID NO:9023 | 60/218,950 | 07/14/2000 |
| SEQ ID NO:9024 | 60/218,950 | 07/14/2000 |
| SEQ ID NO:9025 | 60/218,950 | 07/14/2000 |
| SEQ ID NO:9026 | 60/218,950 | 07/14/2000 |
| SEQ ID NO:9027 | 60/218,950 | 07/14/2000 |
| SEQ ID NO:9028 | 60/218,950 | 07/14/2000 |
| SEQ ID NO:9029 | 60/218,950 | 07/14/2000 |
| SEQ ID NO:9030 | 60/218,950 | 07/14/2000 |
| SEQ ID NO:9031 | 60/218,950 | 07/14/2000 |
| SEQ ID NO:9032 | 60/218,950 | 07/14/2000 |
| SEQ ID NO:9033 | 60/218,950 | 07/14/2000 |
| SEQ ID NO:9034 | 60/218,950 | 07/14/2000 |
| SEQ ID NO:9035 | 60/218,950 | 07/14/2000 |
| SEQ ID NO:9036 | 60/218,950 | 07/14/2000 |
| SEQ ID NO:9037 | 60/218,950 | 07/14/2000 |
| SEQ ID NO:9038 | 60/218,950 | 07/14/2000 |
| SEQ ID NO:9039 | 60/218,950 | 07/14/2000 |
| SEQ ID NO:9040 | 60/218,950 | 07/14/2000 |
| SEQ ID NO:9041 | 60/218,950 | 07/14/2000 |
| SEQ ID NO:9042 | 60/218,950 | 07/14/2000 |
| SEQ ID NO:9043 | 60/218,950 | 07/14/2000 |
| SEQ ID NO:9044 | 60/218,950 | 07/14/2000 |
| SEQ ID NO:9045 | 60/218,950 | 07/14/2000 |
| SEQ ID NO:9046 | 60/218,950 | 07/14/2000 |
| SEQ ID NO:9047 | 60/218,950 | 07/14/2000 |
| SEQ ID NO:9048 | 60/218,950 | 07/14/2000 |
| SEQ ID NO:9049 | 60/218,950 | 07/14/2000 |
| SEQ ID NO:9050 | 60/218,950 | 07/14/2000 |
| SEQ ID NO:9051 | 60/218,950 | 07/14/2000 |
| SEQ ID NO:9052 | 60/223,378 | 08/07/2000 |
| SEQ ID NO:9053 | 60/223,378 | 08/07/2000 |
| SEQ ID NO:9054 | 60/223,378 | 08/07/2000 |
| SEQ ID NO:9055 | 60/223,378 | 08/07/2000 |
| SEQ ID NO:9056 | 60/223,378 | 08/07/2000 |
| SEQ ID NO:9057 | 60/223,378 | 08/07/2000 |
| SEQ ID NO:9058 | 60/223,378 | 08/07/2000 |
| SEQ ID NO:9059 | 60/223,378 | 08/07/2000 |
| SEQ ID NO:9060 | 60/223,378 | 08/07/2000 |
| SEQ ID NO:9061 | 60/223,378 | 08/07/2000 |
| SEQ ID NO:9062 | 60/223,378 | 08/07/2000 |
| SEQ ID NO:9063 | 60/223,378 | 08/07/2000 |
| SEQ ID NO:9064 | 60/223,378 | 08/07/2000 |
| SEQ ID NO:9065 | 60/223,378 | 08/07/2000 |
| SEQ ID NO:9066 | 60/223,378 | 08/07/2000 |
| SEQ ID NO:9067 | 60/223,378 | 08/07/2000 |
| SEQ ID NO:9068 | 60/223,378 | 08/07/2000 |
| SEQ ID NO:9069 | 60/223,378 | 08/07/2000 |
| SEQ ID NO:9070 | 60/223,378 | 08/07/2000 |
| SEQ ID NO:9071 | 60/223,378 | 08/07/2000 |
| SEQ ID NO:9072 | 60/223,378 | 08/07/2000 |
| SEQ ID NO:9073 | 60/223,378 | 08/07/2000 |
| SEQ ID NO:9074 | 60/223,378 | 08/07/2000 |
| SEQ ID NO:9075 | 60/223,378 | 08/07/2000 |
| SEQ ID NO:9076 | 60/223,378 | 08/07/2000 |
| SEQ ID NO:9077 | 60/223,378 | 08/07/2000 |
| SEQ ID NO:9078 | 60/223,378 | 08/07/2000 |
| SEQ ID NO:9079 | 60/223,378 | 08/07/2000 |
| SEQ ID NO:9080 | 60/223,378 | 08/07/2000 |
| SEQ ID NO:9081 | 60/223,378 | 08/07/2000 |
| SEQ ID NO:9082 | 60/223,378 | 08/07/2000 |
| SEQ ID NO:9083 | 60/223,378 | 08/07/2000 |
| SEQ ID NO:9084 | 60/223,378 | 08/07/2000 |
| SEQ ID NO:9085 | 60/223,378 | 08/07/2000 |
| SEQ ID NO:9086 | 60/223,378 | 08/07/2000 |
| SEQ ID NO:9087 | 60/223,378 | 08/07/2000 |
| SEQ ID NO:9088 | 60/223,378 | 08/07/2000 |
| SEQ ID NO:9089 | 60/223,378 | 08/07/2000 |
| SEQ ID NO:9090 | 60/223,378 | 08/07/2000 |
| SEQ ID NO:9091 | 60/223,378 | 08/07/2000 |
| SEQ ID NO:9092 | 60/223,378 | 08/07/2000 |
| SEQ ID NO:9093 | 60/223,378 | 08/07/2000 |
| SEQ ID NO:9094 | 60/223,378 | 08/07/2000 |
| SEQ ID NO:9095 | 60/223,378 | 08/07/2000 |
| SEQ ID NO:9096 | 60/223,378 | 08/07/2000 |
| SEQ ID NO:9097 | 60/223,378 | 08/07/2000 |
| SEQ ID NO:9098 | 60/223,378 | 08/07/2000 |
| SEQ ID NO:9099 | 60/223,378 | 08/07/2000 |
| SEQ ID NO:9100 | 60/223,378 | 08/07/2000 |
| SEQ ID NO:9101 | 60/223,378 | 08/07/2000 |
| SEQ ID NO:9102 | 60/223,378 | 08/07/2000 |
| SEQ ID NO:9103 | 60/223,378 | 08/07/2000 |
| SEQ ID NO:9104 | 60/223,378 | 08/07/2000 |
| SEQ ID NO:9105 | 60/223,378 | 08/07/2000 |
| SEQ ID NO:9106 | 60/223,378 | 08/07/2000 |
| SEQ ID NO:9107 | 60/223,378 | 08/07/2000 |
| SEQ ID NO:9108 | 60/223,378 | 08/07/2000 |
| SEQ ID NO:9109 | 60/223,378 | 08/07/2000 |
| SEQ ID NO:9110 | 60/223,378 | 08/07/2000 |
| SEQ ID NO:9111 | 60/223,378 | 08/07/2000 |
| SEQ ID NO:9112 | 60/223,378 | 08/07/2000 |
| SEQ ID NO:9113 | 60/223,378 | 08/07/2000 |
| SEQ ID NO:9114 | 60/223,378 | 08/07/2000 |
| SEQ ID NO:9115 | 60/223,378 | 08/07/2000 |
| SEQ ID NO:9116 | 60/223,378 | 08/07/2000 |
| SEQ ID NO:9117 | 60/223,378 | 08/07/2000 |
| SEQ ID NO:9118 | 60/223,378 | 08/07/2000 |
| SEQ ID NO:9119 | 60/223,378 | 08/07/2000 |
| SEQ ID NO:9120 | 60/223,378 | 08/07/2000 |
| SEQ ID NO:9121 | 60/223,378 | 08/07/2000 |
| SEQ ID NO:9122 | 60/223,378 | 08/07/2000 |
| SEQ ID NO:9123 | 60/223,378 | 08/07/2000 |
| SEQ ID NO:9124 | 60/223,378 | 08/07/2000 |
| SEQ ID NO:9125 | 60/223,378 | 08/07/2000 |
| SEQ ID NO:9126 | 60/223,378 | 08/07/2000 |
| SEQ ID NO:9127 | 60/223,378 | 08/07/2000 |
| SEQ ID NO:9128 | 60/223,378 | 08/07/2000 |
| SEQ ID NO:9129 | 60/223,378 | 08/07/2000 |
| SEQ ID NO:9130 | 60/223,378 | 08/07/2000 |
| SEQ ID NO:9131 | 60/223,378 | 08/07/2000 |
| SEQ ID NO:9132 | 60/223,378 | 08/07/2000 |
| SEQ ID NO:9133 | 60/223,378 | 08/07/2000 |
| SEQ ID NO:9134 | 60/223,378 | 08/07/2000 |
| SEQ ID NO:9135 | 60/223,378 | 08/07/2000 |
| SEQ ID NO:9136 | 60/223,378 | 08/07/2000 |
| SEQ ID NO:9137 | 60/223,378 | 08/07/2000 |
| SEQ ID NO:9138 | 60/223,378 | 08/07/2000 |
| SEQ ID NO:9139 | 60/223,378 | 08/07/2000 |
| SEQ ID NO:9140 | 60/223,378 | 08/07/2000 |
| SEQ ID NO:9141 | 60/223,378 | 08/07/2000 |
| SEQ ID NO:9142 | 60/223,378 | 08/07/2000 |
| SEQ ID NO:9143 | 60/223,378 | 08/07/2000 |
| SEQ ID NO:9144 | 60/223,378 | 08/07/2000 |
| SEQ ID NO:9145 | 60/223,378 | 08/07/2000 |
| SEQ ID NO:9146 | 60/223,378 | 08/07/2000 |
| SEQ ID NO:9147 | 60/223,378 | 08/07/2000 |
| SEQ ID NO:9148 | 60/223,378 | 08/07/2000 |
| SEQ ID NO:9149 | 60/223,378 | 08/07/2000 |
| SEQ ID NO:9150 | 60/223,378 | 08/07/2000 |
| SEQ ID NO:9151 | 60/223,378 | 08/07/2000 |
| SEQ ID NO:9152 | 60/223,378 | 08/07/2000 |
| SEQ ID NO:9153 | 60/223,378 | 08/07/2000 |
| SEQ ID NO:9154 | 60/223,378 | 08/07/2000 |
| SEQ ID NO:9155 | 60/223,378 | 08/07/2000 |
| SEQ ID NO:9156 | 60/223,378 | 08/07/2000 |
| SEQ ID NO:9157 | 60/223,378 | 08/07/2000 |
| SEQ ID NO:9158 | 60/223,378 | 08/07/2000 |
| SEQ ID NO:9159 | 60/223,378 | 08/07/2000 |
| SEQ ID NO:9160 | 60/223,378 | 08/07/2000 |
| SEQ ID NO:9161 | 60/223,378 | 08/07/2000 |
| SEQ ID NO:9162 | 60/223,378 | 08/07/2000 |
| SEQ ID NO:9163 | 60/223,378 | 08/07/2000 |
| SEQ ID NO:9164 | 60/223,378 | 08/07/2000 |
| SEQ ID NO:9165 | 60/223,378 | 08/07/2000 |
| SEQ ID NO:9166 | 60/223,378 | 08/07/2000 |
| SEQ ID NO:9167 | 60/223,378 | 08/07/2000 |
| SEQ ID NO:9168 | 60/223,378 | 08/07/2000 |
| SEQ ID NO:9169 | 60/223,378 | 08/07/2000 |
| SEQ ID NO:9170 | 60/223,378 | 08/07/2000 |
| SEQ ID NO:9171 | 60/223,378 | 08/07/2000 |
| SEQ ID NO:9172 | 60/223,378 | 08/07/2000 |
| SEQ ID NO:9173 | 60/223,378 | 08/07/2000 |
| SEQ ID NO:9174 | 60/223,378 | 08/07/2000 |
| SEQ ID NO:9175 | 60/223,378 | 08/07/2000 |
| SEQ ID NO:9176 | 60/223,378 | 08/07/2000 |
| SEQ ID NO:9177 | 60/223,378 | 08/07/2000 |
| SEQ ID NO:9178 | 60/223,378 | 08/07/2000 |
| SEQ ID NO:9179 | 60/223,378 | 08/07/2000 |
| SEQ ID NO:9180 | 60/223,378 | 08/07/2000 |
| SEQ ID NO:9181 | 60/223,378 | 08/07/2000 |
| SEQ ID NO:9182 | 60/223,378 | 08/07/2000 |
| SEQ ID NO:9183 | 60/223,378 | 08/07/2000 |
| SEQ ID NO:9184 | 60/223,378 | 08/07/2000 |
| SEQ ID NO:9185 | 60/223,378 | 08/07/2000 |
| SEQ ID NO:9186 | 60/223,378 | 08/07/2000 |
| SEQ ID NO:9187 | 60/223,378 | 08/07/2000 |
| SEQ ID NO:9188 | 60/223,378 | 08/07/2000 |
| SEQ ID NO:9189 | 60/223,378 | 08/07/2000 |
| SEQ ID NO:9190 | 60/223,378 | 08/07/2000 |
| SEQ ID NO:9191 | 60/223,378 | 08/07/2000 |
| SEQ ID NO:9192 | 60/223,378 | 08/07/2000 |
| SEQ ID NO:9193 | 60/223,378 | 08/07/2000 |
| SEQ ID NO:9194 | 60/223,378 | 08/07/2000 |
| SEQ ID NO:9195 | 60/223,378 | 08/07/2000 |
| SEQ ID NO:9196 | 60/223,378 | 08/07/2000 |
| SEQ ID NO:9197 | 60/223,378 | 08/07/2000 |
| SEQ ID NO:9198 | 60/222,903 | 08/03/2000 |
| SEQ ID NO:9199 | 60/222,903 | 08/03/2000 |
| SEQ ID NO:9200 | 60/222,903 | 08/03/2000 |
| SEQ ID NO:9201 | 60/222,903 | 08/03/2000 |
| SEQ ID NO:9202 | 60/222,903 | 08/03/2000 |
| SEQ ID NO:9203 | 60/222,903 | 08/03/2000 |
| SEQ ID NO:9204 | 60/222,903 | 08/03/2000 |
| SEQ ID NO:9205 | 60/222,903 | 08/03/2000 |
| SEQ ID NO:9206 | 60/222,903 | 08/03/2000 |
| SEQ ID NO:9207 | 60/222,903 | 08/03/2000 |
| SEQ ID NO:9208 | 60/222,903 | 08/03/2000 |
| SEQ ID NO:9209 | 60/222,903 | 08/03/2000 |
| SEQ ID NO:9210 | 60/222,903 | 08/03/2000 |
| SEQ ID NO:9211 | 60/222,903 | 08/03/2000 |
| SEQ ID NO:9212 | 60/222,903 | 08/03/2000 |
| SEQ ID NO:9213 | 60/222,903 | 08/03/2000 |
| SEQ ID NO:9214 | 60/222,903 | 08/03/2000 |
| SEQ ID NO:9215 | 60/222,903 | 08/03/2000 |
| SEQ ID NO:9216 | 60/222,903 | 08/03/2000 |
| SEQ ID NO:9217 | 60/222,903 | 08/03/2000 |
| SEQ ID NO:9218 | 60/222,903 | 08/03/2000 |
| SEQ ID NO:9219 | 60/222,903 | 08/03/2000 |
| SEQ ID NO:9220 | 60/222,903 | 08/03/2000 |
| SEQ ID NO:9221 | 60/222,903 | 08/03/2000 |
| SEQ ID NO:9222 | 60/222,903 | 08/03/2000 |
| SEQ ID NO:9223 | 60/222,903 | 08/03/2000 |
| SEQ ID NO:9224 | 60/222,903 | 08/03/2000 |
| SEQ ID NO:9225 | 60/222,903 | 08/03/2000 |
| SEQ ID NO:9226 | 60/222,903 | 08/03/2000 |
| SEQ ID NO:9227 | 60/222,903 | 08/03/2000 |
| SEQ ID NO:9228 | 60/222,903 | 08/03/2000 |
| SEQ ID NO:9229 | 60/222,903 | 08/03/2000 |
| SEQ ID NO:9230 | 60/222,903 | 08/03/2000 |
| SEQ ID NO:9231 | 60/222,903 | 08/03/2000 |
| SEQ ID NO:9232 | 60/222,903 | 08/03/2000 |
| SEQ ID NO:9233 | 60/222,903 | 08/03/2000 |
| SEQ ID NO:9234 | 60/222,903 | 08/03/2000 |
| SEQ ID NO:9235 | 60/222,903 | 08/03/2000 |
| SEQ ID NO:9236 | 60/222,903 | 08/03/2000 |
| SEQ ID NO:9237 | 60/222,903 | 08/03/2000 |
| SEQ ID NO:9238 | 60/222,903 | 08/03/2000 |
| SEQ ID NO:9239 | 60/222,903 | 08/03/2000 |
| SEQ ID NO:9240 | 60/222,903 | 08/03/2000 |
| SEQ ID NO:9241 | 60/222,903 | 08/03/2000 |
| SEQ ID NO:9242 | 60/222,903 | 08/03/2000 |
| SEQ ID NO:9243 | 60/222,903 | 08/03/2000 |
| SEQ ID NO:9244 | 60/222,903 | 08/03/2000 |
| SEQ ID NO:9245 | 60/222,903 | 08/03/2000 |
| SEQ ID NO:9246 | 60/222,903 | 08/03/2000 |
| SEQ ID NO:9247 | 60/222,903 | 08/03/2000 |
| SEQ ID NO:9248 | 60/222,903 | 08/03/2000 |
| SEQ ID NO:9249 | 60/222,903 | 08/03/2000 |
| SEQ ID NO:9250 | 60/222,903 | 08/03/2000 |
| SEQ ID NO:9251 | 60/222,903 | 08/03/2000 |
| SEQ ID NO:9252 | 60/222,903 | 08/03/2000 |
| SEQ ID NO:9253 | 60/222,903 | 08/03/2000 |
| SEQ ID NO:9254 | 60/222,903 | 08/03/2000 |
| SEQ ID NO:9255 | 60/222,903 | 08/03/2000 |
| SEQ ID NO:9256 | 60/222,903 | 08/03/2000 |
| SEQ ID NO:9257 | 60/222,903 | 08/03/2000 |
| SEQ ID NO:9258 | 60/222,903 | 08/03/2000 |
| SEQ ID NO:9259 | 60/222,903 | 08/03/2000 |
| SEQ ID NO:9260 | 60/222,903 | 08/03/2000 |
| SEQ ID NO:9261 | 60/222,903 | 08/03/2000 |
| SEQ ID NO:9262 | 60/222,903 | 08/03/2000 |
| SEQ ID NO:9263 | 60/222,903 | 08/03/2000 |
| SEQ ID NO:9264 | 60/222,903 | 08/03/2000 |
| SEQ ID NO:9265 | 60/222,903 | 08/03/2000 |
| SEQ ID NO:9266 | 60/222,903 | 08/03/2000 |
| SEQ ID NO:9267 | 60/222,903 | 08/03/2000 |
| SEQ ID NO:9268 | 60/222,903 | 08/03/2000 |
| SEQ ID NO:9269 | 60/222,903 | 08/03/2000 |
| SEQ ID NO:9270 | 60/222,903 | 08/03/2000 |
| SEQ ID NO:9271 | 60/222,903 | 08/03/2000 |
| SEQ ID NO:9272 | 60/222,903 | 08/03/2000 |
| SEQ ID NO:9273 | 60/222,903 | 08/03/2000 |
| SEQ ID NO:9274 | 60/222,903 | 08/03/2000 |
| SEQ ID NO:9275 | 60/222,903 | 08/03/2000 |
| SEQ ID NO:9276 | 60/222,903 | 08/03/2000 |
| SEQ ID NO:9277 | 60/222,903 | 08/03/2000 |
| SEQ ID NO:9278 | 60/222,903 | 08/03/2000 |
| SEQ ID NO:9279 | 60/222,903 | 08/03/2000 |
| SEQ ID NO:9280 | 60/222,903 | 08/03/2000 |
| SEQ ID NO:9281 | 60/222,903 | 08/03/2000 |
| SEQ ID NO:9282 | 60/222,903 | 08/03/2000 |
| SEQ ID NO:9283 | 60/222,903 | 08/03/2000 |
| SEQ ID NO:9284 | 60/222,903 | 08/03/2000 |
| SEQ ID NO:9285 | 60/222,903 | 08/03/2000 |
| SEQ ID NO:9286 | 60/222,903 | 08/03/2000 |
| SEQ ID NO:9287 | 60/222,903 | 08/03/2000 |
| SEQ ID NO:9288 | 60/222,903 | 08/03/2000 |
| SEQ ID NO:9289 | 60/222,903 | 08/03/2000 |
| SEQ ID NO:9290 | 60/222,903 | 08/03/2000 |
| SEQ ID NO:9291 | 60/222,903 | 08/03/2000 |
| SEQ ID NO:9292 | 60/222,903 | 08/03/2000 |
| SEQ ID NO:9293 | 60/222,903 | 08/03/2000 |
| SEQ ID NO:9294 | 60/222,903 | 08/03/2000 |
| SEQ ID NO:9295 | 60/222,903 | 08/03/2000 |
| SEQ ID NO:9296 | 60/222,903 | 08/03/2000 |
| SEQ ID NO:9297 | 60/222,903 | 08/03/2000 |
| SEQ ID NO:9298 | 60/222,903 | 08/03/2000 |
| SEQ ID NO:9299 | 60/222,903 | 08/03/2000 |
| SEQ ID NO:9300 | 60/222,903 | 08/03/2000 |
| SEQ ID NO:9301 | 60/222,903 | 08/03/2000 |
| SEQ ID NO:9302 | 60/222,903 | 08/03/2000 |
| SEQ ID NO:9303 | 60/222,903 | 08/03/2000 |
| SEQ ID NO:9304 | 60/222,903 | 08/03/2000 |
| SEQ ID NO:9305 | 60/222,903 | 08/03/2000 |
| SEQ ID NO:9306 | 60/222,903 | 08/03/2000 |
| SEQ ID NO:9307 | 60/222,903 | 08/03/2000 |
| SEQ ID NO:9308 | 60/222,903 | 08/03/2000 |
| SEQ ID NO:9309 | 60/222,903 | 08/03/2000 |
| SEQ ID NO:9310 | 60/222,903 | 08/03/2000 |
| SEQ ID NO:9311 | 60/222,903 | 08/03/2000 |
| SEQ ID NO:9312 | 60/222,903 | 08/03/2000 |
| SEQ ID NO:9313 | 60/222,903 | 08/03/2000 |
| SEQ ID NO:9314 | 60/222,903 | 08/03/2000 |
| SEQ ID NO:9315 | 60/222,903 | 08/03/2000 |
| SEQ ID NO:9316 | 60/222,903 | 08/03/2000 |
| SEQ ID NO:9317 | 60/222,903 | 08/03/2000 |
| SEQ ID NO:9318 | 60/222,903 | 08/03/2000 |
| SEQ ID NO:9319 | 60/222,903 | 08/03/2000 |
| SEQ ID NO:9320 | 60/222,903 | 08/03/2000 |
| SEQ ID NO:9321 | 60/222,903 | 08/03/2000 |
| SEQ ID NO:9322 | 60/222,903 | 08/03/2000 |
| SEQ ID NO:9323 | 60/222,903 | 08/03/2000 |
| SEQ ID NO:9324 | 60/222,903 | 08/03/2000 |
| SEQ ID NO:9325 | 60/222,903 | 08/03/2000 |
| SEQ ID NO:9326 | 60/222,903 | 08/03/2000 |
| SEQ ID NO:9327 | 60/222,903 | 08/03/2000 |
| SEQ ID NO:9328 | 60/222,903 | 08/03/2000 |
| SEQ ID NO:9329 | 60/222,903 | 08/03/2000 |
| SEQ ID NO:9330 | 60/222,903 | 08/03/2000 |
| SEQ ID NO:9331 | 60/222,903 | 08/03/2000 |
| SEQ ID NO:9332 | 60/222,903 | 08/03/2000 |
| SEQ ID NO:9333 | 60/222,903 | 08/03/2000 |
| SEQ ID NO:9334 | 60/222,903 | 08/03/2000 |
| SEQ ID NO:9335 | 60/222,903 | 08/03/2000 |
| SEQ ID NO:9336 | 60/222,903 | 08/03/2000 |
| SEQ ID NO:9337 | 60/222,903 | 08/03/2000 |
| SEQ ID NO:9338 | 60/222,903 | 08/03/2000 |
| SEQ ID NO:9339 | 60/222,903 | 08/03/2000 |
| SEQ ID NO:9340 | 60/222,903 | 08/03/2000 |
| SEQ ID NO:9341 | 60/222,903 | 08/03/2000 |
| SEQ ID NO:9342 | 60/222,903 | 08/03/2000 |
| SEQ ID NO:9343 | 60/222,903 | 08/03/2000 |
| SEQ ID NO:9344 | 60/222,903 | 08/03/2000 |
| SEQ ID NO:9345 | 60/222,903 | 08/03/2000 |
| SEQ ID NO:9346 | 60/222,903 | 08/03/2000 |
| SEQ ID NO:9347 | 60/222,903 | 08/03/2000 |
| SEQ ID NO:9348 | 60/222,903 | 08/03/2000 |
| SEQ ID NO:9349 | 60/222,903 | 08/03/2000 |
| SEQ ID NO:9350 | 60/222,903 | 08/03/2000 |
| SEQ ID NO:9351 | 60/222,903 | 08/03/2000 |
| SEQ ID NO:9352 | 60/222,903 | 08/03/2000 |
| SEQ ID NO:9353 | 60/222,903 | 08/03/2000 |
| SEQ ID NO:9354 | 60/222,903 | 08/03/2000 |
| SEQ ID NO:9355 | 60/222,903 | 08/03/2000 |
| SEQ ID NO:9356 | 60/222,903 | 08/03/2000 |
| SEQ ID NO:9357 | 60/222,903 | 08/03/2000 |
| SEQ ID NO:9358 | 60/222,903 | 08/03/2000 |
| SEQ ID NO:9359 | 60/222,903 | 08/03/2000 |
| SEQ ID NO:9360 | 60/222,903 | 08/03/2000 |
| SEQ ID NO:9361 | 60/222,903 | 08/03/2000 |
| SEQ ID NO:9362 | 60/222,903 | 08/03/2000 |
| SEQ ID NO:9363 | 60/222,903 | 08/03/2000 |
| SEQ ID NO:9364 | 60/222,903 | 08/03/2000 |
| SEQ ID NO:9365 | 60/222,903 | 08/03/2000 |
| SEQ ID NO:9366 | 60/222,903 | 08/03/2000 |
| SEQ ID NO:9367 | 60/222,903 | 08/03/2000 |
| SEQ ID NO:9368 | 60/222,903 | 08/03/2000 |
| SEQ ID NO:9369 | 60/222,903 | 08/03/2000 |
| SEQ ID NO:9370 | 60/222,903 | 08/03/2000 |
| SEQ ID NO:9371 | 60/222,903 | 08/03/2000 |
| SEQ ID NO:9372 | 60/222,903 | 08/03/2000 |
| SEQ ID NO:9373 | 60/222,903 | 08/03/2000 |
| SEQ ID NO:9374 | 60/222,903 | 08/03/2000 |
| SEQ ID NO:9375 | 60/222,903 | 08/03/2000 |
| SEQ ID NO:9376 | 60/222,903 | 08/03/2000 |
| SEQ ID NO:9377 | 60/222,903 | 08/03/2000 |
| SEQ ID NO:9378 | 60/222,903 | 08/03/2000 |
| SEQ ID NO:9379 | 60/222,903 | 08/03/2000 |
| SEQ ID NO:9380 | 60/222,903 | 08/03/2000 |
| SEQ ID NO:9381 | 60/222,903 | 08/03/2000 |
| SEQ ID NO:9382 | 60/222,903 | 08/03/2000 |
| SEQ ID NO:9383 | 60/222,903 | 08/03/2000 |
| SEQ ID NO:9384 | 60/222,903 | 08/03/2000 |
| SEQ ID NO:9385 | 60/222,903 | 08/03/2000 |
| SEQ ID NO:9386 | 60/222,903 | 08/03/2000 |
| SEQ ID NO:9387 | 60/222,903 | 08/03/2000 |
| SEQ ID NO:9388 | 60/222,903 | 08/03/2000 |
| SEQ ID NO:9389 | 60/222,903 | 08/03/2000 |
| SEQ ID NO:9390 | 60/222,903 | 08/03/2000 |
| SEQ ID NO:9391 | 60/222,903 | 08/03/2000 |
| SEQ ID NO:9392 | 60/222,903 | 08/03/2000 |
| SEQ ID NO:9393 | 60/222,903 | 08/03/2000 |
| SEQ ID NO:9394 | 60/222,903 | 08/03/2000 |
| SEQ ID NO:9395 | 60/222,903 | 08/03/2000 |
| SEQ ID NO:9396 | 60/222,903 | 08/03/2000 |
| SEQ ID NO:9397 | 60/222,903 | 08/03/2000 |
| SEQ ID NO:9398 | 60/222,903 | 08/03/2000 |
| SEQ ID NO:9399 | 60/222,903 | 08/03/2000 |
| SEQ ID NO:9400 | 60/222,903 | 08/03/2000 |
| SEQ ID NO:9401 | 60/222,903 | 08/03/2000 |
| SEQ ID NO:9402 | 60/222,903 | 08/03/2000 |
| SEQ ID NO:9403 | 60/222,903 | 08/03/2000 |
| SEQ ID NO:9404 | 60/222,903 | 08/03/2000 |
| SEQ ID NO:9405 | 60/222,903 | 08/03/2000 |
| SEQ ID NO:9406 | 60/222,903 | 08/03/2000 |
| SEQ ID NO:9407 | 60/222,903 | 08/03/2000 |
| SEQ ID NO:9408 | 60/222,903 | 08/03/2000 |
| SEQ ID NO:9409 | 60/222,903 | 08/03/2000 |
| SEQ ID NO:9410 | 60/222,903 | 08/03/2000 |
| SEQ ID NO:9411 | 60/222,903 | 08/03/2000 |
| SEQ ID NO:9412 | 60/222,903 | 08/03/2000 |
| SEQ ID NO:9413 | 60/222,903 | 08/03/2000 |
| SEQ ID NO:9414 | 60/222,903 | 08/03/2000 |
| SEQ ID NO:9415 | 60/222,903 | 08/03/2000 |
| SEQ ID NO:9416 | 60/222,903 | 08/03/2000 |
| SEQ ID NO:9417 | 60/222,903 | 08/03/2000 |
| SEQ ID NO:9418 | 60/222,903 | 08/03/2000 |
| SEQ ID NO:9419 | 60/222,903 | 08/03/2000 |
| SEQ ID NO:9420 | 60/222,903 | 08/03/2000 |
| SEQ ID NO:9421 | 60/222,903 | 08/03/2000 |
| SEQ ID NO:9422 | 60/222,903 | 08/03/2000 |
| SEQ ID NO:9423 | 60/222,903 | 08/03/2000 |
| SEQ ID NO:9424 | 60/222,903 | 08/03/2000 |
| SEQ ID NO:9425 | 60/222,903 | 08/03/2000 |
| SEQ ID NO:9426 | 60/222,903 | 08/03/2000 |
| SEQ ID NO:9427 | 60/222,903 | 08/03/2000 |
| SEQ ID NO:9428 | 60/222,903 | 08/03/2000 |
| SEQ ID NO:9429 | 60/222,903 | 08/03/2000 |
| SEQ ID NO:9430 | 60/222,903 | 08/03/2000 |
| SEQ ID NO:9431 | 60/222,903 | 08/03/2000 |
| SEQ ID NO:9432 | 60/222,903 | 08/03/2000 |
| SEQ ID NO:9433 | 60/222,903 | 08/03/2000 |
| SEQ ID NO:9434 | 60/222,903 | 08/03/2000 |
| SEQ ID NO:9435 | 60/222,903 | 08/03/2000 |
| SEQ ID NO:9436 | 60/222,903 | 08/03/2000 |
| SEQ ID NO:9437 | 60/222,903 | 08/03/2000 |
| SEQ ID NO:9438 | 60/222,903 | 08/03/2000 |
| SEQ ID NO:9439 | 60/222,903 | 08/03/2000 |
| SEQ ID NO:9440 | 60/222,903 | 08/03/2000 |
| SEQ ID NO:9441 | 60/222,903 | 08/03/2000 |
| SEQ ID NO:9442 | 60/222,903 | 08/03/2000 |
| SEQ ID NO:9443 | 60/222,903 | 08/03/2000 |
| SEQ ID NO:9444 | 60/222,903 | 08/03/2000 |
| SEQ ID NO:9445 | 60/222,903 | 08/03/2000 |
| SEQ ID NO:9446 | 60/222,903 | 08/03/2000 |
| SEQ ID NO:9447 | 60/222,903 | 08/03/2000 |
| SEQ ID NO:9448 | 60/222,903 | 08/03/2000 |
| SEQ ID NO:9449 | 60/222,903 | 08/03/2000 |
| SEQ ID NO:9450 | 60/222,903 | 08/03/2000 |
| SEQ ID NO:9451 | 60/222,903 | 08/03/2000 |
| SEQ ID NO:9452 | 60/222,903 | 08/03/2000 |
| SEQ ID NO:9453 | 60/222,903 | 08/03/2000 |
| SEQ ID NO:9454 | 60/222,903 | 08/03/2000 |
| SEQ ID NO:9455 | 60/222,903 | 08/03/2000 |
| SEQ ID NO:9456 | 60/222,903 | 08/03/2000 |
| SEQ ID NO:9457 | 60/222,903 | 08/03/2000 |
| SEQ ID NO:9458 | 60/222,903 | 08/03/2000 |
| SEQ ID NO:9459 | 60/222,903 | 08/03/2000 |
| SEQ ID NO:9460 | 60/222,903 | 08/03/2000 |
| SEQ ID NO:9461 | 60/222,903 | 08/03/2000 |
| SEQ ID NO:9462 | 60/222,903 | 08/03/2000 |
| SEQ ID NO:9463 | 60/222,903 | 08/03/2000 |
| SEQ ID NO:9464 | 60/222,903 | 08/03/2000 |
| SEQ ID NO:9465 | 60/222,903 | 08/03/2000 |
| SEQ ID NO:9466 | 60/222,903 | 08/03/2000 |
| SEQ ID NO:9467 | 60/222,903 | 08/03/2000 |
| SEQ ID NO:9468 | 60/222,903 | 08/03/2000 |
| SEQ ID NO:9469 | 60/222,903 | 08/03/2000 |
| SEQ ID NO:9470 | 60/222,903 | 08/03/2000 |
| SEQ ID NO:9471 | 60/222,903 | 08/03/2000 |
| SEQ ID NO:9472 | 60/222,903 | 08/03/2000 |
| SEQ ID NO:9473 | 60/222,903 | 08/03/2000 |
| SEQ ID NO:9474 | 60/222,903 | 08/03/2000 |
| SEQ ID NO:9475 | 60/222,903 | 08/03/2000 |
| SEQ ID NO:9476 | 60/222,903 | 08/03/2000 |
| SEQ ID NO:9477 | 60/222,903 | 08/03/2000 |
| SEQ ID NO:9478 | 60/222,903 | 08/03/2000 |
| SEQ ID NO:9479 | 60/222,903 | 08/03/2000 |
| SEQ ID NO:9480 | 60/222,903 | 08/03/2000 |
| SEQ ID NO:9481 | 60/222,903 | 08/03/2000 |
| SEQ ID NO:9482 | 60/222,903 | 08/03/2000 |
| SEQ ID NO:9483 | 60/222,903 | 08/03/2000 |
| SEQ ID NO:9484 | 60/222,903 | 08/03/2000 |
| SEQ ID NO:9485 | 60/222,903 | 08/03/2000 |
| SEQ ID NO:9486 | 60/222,903 | 08/03/2000 |
| SEQ ID NO:9487 | 60/222,903 | 08/03/2000 |
| SEQ ID NO:9488 | 60/222,903 | 08/03/2000 |
| SEQ ID NO:9489 | 60/222,903 | 08/03/2000 |
| SEQ ID NO:9490 | 60/222,903 | 08/03/2000 |
| SEQ ID NO:9491 | 60/222,903 | 08/03/2000 |
| SEQ ID NO:9492 | 60/222,903 | 08/03/2000 |
| SEQ ID NO:9493 | 60/222,903 | 08/03/2000 |
| SEQ ID NO:9494 | 60/222,903 | 08/03/2000 |
| SEQ ID NO:9495 | 60/222,903 | 08/03/2000 |
| SEQ ID NO:9496 | 60/222,903 | 08/03/2000 |
| SEQ ID NO:9497 | 60/222,903 | 08/03/2000 |
| SEQ ID NO:9498 | 60/222,903 | 08/03/2000 |
| SEQ ID NO:9499 | 60/222,903 | 08/03/2000 |
| SEQ ID NO:9500 | 60/222,903 | 08/03/2000 |
| SEQ ID NO:9501 | 60/222,903 | 08/03/2000 |
| SEQ ID NO:9502 | 60/222,903 | 08/03/2000 |
| SEQ ID NO:9503 | 60/222,903 | 08/03/2000 |
| SEQ ID NO:9504 | 60/222,903 | 08/03/2000 |
| SEQ ID NO:9505 | 60/222,903 | 08/03/2000 |
| SEQ ID NO:9506 | 60/222,903 | 08/03/2000 |
| SEQ ID NO:9507 | 60/222,903 | 08/03/2000 |
| SEQ ID NO:9508 | 60/222,903 | 08/03/2000 |
| SEQ ID NO:9509 | 60/222,903 | 08/03/2000 |
| SEQ ID NO:9510 | 60/222,903 | 08/03/2000 |
| SEQ ID NO:9511 | 60/222,903 | 08/03/2000 |
| SEQ ID NO:9512 | 60/222,903 | 08/03/2000 |
| SEQ ID NO:9513 | 60/222,903 | 08/03/2000 |
| SEQ ID NO:9514 | 60/222,903 | 08/03/2000 |
| SEQ ID NO:9515 | 60/222,903 | 08/03/2000 |
| SEQ ID NO:9516 | 60/222,903 | 08/03/2000 |
| SEQ ID NO:9517 | 60/222,903 | 08/03/2000 |
| SEQ ID NO:9518 | 60/222,903 | 08/03/2000 |
| SEQ ID NO:9519 | 60/222,903 | 08/03/2000 |
| SEQ ID NO:9520 | 60/222,903 | 08/03/2000 |
| SEQ ID NO:9521 | 60/222,903 | 08/03/2000 |
| SEQ ID NO:9522 | 60/222,903 | 08/03/2000 |
| SEQ ID NO:9523 | 60/222,903 | 08/03/2000 |
| SEQ ID NO:9524 | 60/222,903 | 08/03/2000 |
| SEQ ID NO:9525 | 60/222,903 | 08/03/2000 |
| SEQ ID NO:9526 | 60/222,903 | 08/03/2000 |
| SEQ ID NO:9527 | 60/222,903 | 08/03/2000 |
| SEQ ID NO:9528 | 60/222,903 | 08/03/2000 |
| SEQ ID NO:9529 | 60/222,903 | 08/03/2000 |
| SEQ ID NO:9530 | 60/222,903 | 08/03/2000 |
| SEQ ID NO:9531 | 60/222,903 | 08/03/2000 |
| SEQ ID NO:9532 | 60/222,903 | 08/03/2000 |
| SEQ ID NO:9533 | 60/222,903 | 08/03/2000 |
| SEQ ID NO:9534 | 60/222,903 | 08/03/2000 |
| SEQ ID NO:9535 | 60/222,903 | 08/03/2000 |
| SEQ ID NO:9536 | 60/222,903 | 08/03/2000 |
| SEQ ID NO:9537 | 60/222,903 | 08/03/2000 |
| SEQ ID NO:9538 | 60/222,903 | 08/03/2000 |
| SEQ ID NO:9539 | 60/222,903 | 08/03/2000 |
| SEQ ID NO:9540 | 60/222,903 | 08/03/2000 |
| SEQ ID NO:9541 | 60/222,903 | 08/03/2000 |
| SEQ ID NO:9542 | 60/222,903 | 08/03/2000 |
| SEQ ID NO:9543 | 60/222,903 | 08/03/2000 |
| SEQ ID NO:9544 | 60/222,903 | 08/03/2000 |
| SEQ ID NO:9545 | 60/222,903 | 08/03/2000 |
| SEQ ID NO:9546 | 60/222,903 | 08/03/2000 |
| SEQ ID NO:9547 | 60/222,903 | 08/03/2000 |
| SEQ ID NO:9548 | 60/222,903 | 08/03/2000 |
| SEQ ID NO:9549 | 60/222,903 | 08/03/2000 |
| SEQ ID NO:9550 | 60/222,903 | 08/03/2000 |
| SEQ ID NO:9551 | 60/222,903 | 08/03/2000 |
| SEQ ID NO:9552 | 60/222,903 | 08/03/2000 |
| SEQ ID NO:9553 | 60/222,903 | 08/03/2000 |
| SEQ ID NO:9554 | 60/222,903 | 08/03/2000 |
| SEQ ID NO:9555 | 60/222,903 | 08/03/2000 |
| SEQ ID NO:9556 | 60/222,903 | 08/03/2000 |
| SEQ ID NO:9557 | 60/222,903 | 08/03/2000 |
| SEQ ID NO:9558 | 60/222,903 | 08/03/2000 |
| SEQ ID NO:9559 | 60/222,903 | 08/03/2000 |
| SEQ ID NO:9560 | 60/222,903 | 08/03/2000 |
| SEQ ID NO:9561 | 60/222,903 | 08/03/2000 |
| SEQ ID NO:9562 | 60/222,903 | 08/03/2000 |
| SEQ ID NO:9563 | 60/222,903 | 08/03/2000 |
| SEQ ID NO:9564 | 60/222,903 | 08/03/2000 |
| SEQ ID NO:9565 | 60/222,903 | 08/03/2000 |
| SEQ ID NO:9566 | 60/222,903 | 08/03/2000 |
| SEQ ID NO:9567 | 60/222,903 | 08/03/2000 |
| SEQ ID NO:9568 | 60/222,903 | 08/03/2000 |
| SEQ ID NO:9569 | 60/222,903 | 08/03/2000 |
| SEQ ID NO:9570 | 60/222,903 | 08/03/2000 |
| SEQ ID NO:9571 | 60/222,903 | 08/03/2000 |
| SEQ ID NO:9572 | 60/222,903 | 08/03/2000 |
| SEQ ID NO:9573 | 60/222,903 | 08/03/2000 |
| SEQ ID NO:9574 | 60/222,903 | 08/03/2000 |
| SEQ ID NO:9575 | 60/222,903 | 08/03/2000 |
| SEQ ID NO:9576 | 60/222,903 | 08/03/2000 |
| SEQ ID NO:9577 | 60/222,903 | 08/03/2000 |
| SEQ ID NO:9578 | 60/222,903 | 08/03/2000 |
| SEQ ID NO:9579 | 60/222,903 | 08/03/2000 |
| SEQ ID NO:9580 | 60/222,903 | 08/03/2000 |
| SEQ ID NO:9581 | 60/222,903 | 08/03/2000 |
| SEQ ID NO:9582 | 60/222,903 | 08/03/2000 |
| SEQ ID NO:9583 | 60/222,903 | 08/03/2000 |
| SEQ ID NO:9584 | 60/222,903 | 08/03/2000 |
| SEQ ID NO:9585 | 60/222,903 | 08/03/2000 |
| SEQ ID NO:9586 | 60/222,903 | 08/03/2000 |
| SEQ ID NO:9587 | 60/222,903 | 08/03/2000 |
| SEQ ID NO:9588 | 60/222,903 | 08/03/2000 |
| SEQ ID NO:9589 | 60/222,903 | 08/03/2000 |
| SEQ ID NO:9590 | 60/222,903 | 08/03/2000 |
| SEQ ID NO:9591 | 60/222,903 | 08/03/2000 |
| SEQ ID NO:9592 | 60/222,903 | 08/03/2000 |
| SEQ ID NO:9593 | 60/222,903 | 08/03/2000 |
| SEQ ID NO:9594 | 60/222,903 | 08/03/2000 |
| SEQ ID NO:9595 | 60/222,903 | 08/03/2000 |
| SEQ ID NO:9596 | 60/222,903 | 08/03/2000 |
| SEQ ID NO:9597 | 60/222,903 | 08/03/2000 |

### References

The following references, to the extent that they provide exemplary procedural or other details supplementary to those set forth herein, are specifically incorporated herein by reference.

U.S. Pat. No. 3,817,827.

U.S. Pat. No. 3,850,752.

U.S. Pat. No. 3,901,654.

U.S. Pat. No. 3,935,074.

U.S. Pat. No. 3,984,533.

U.S. Pat. No. 3,996,345.

U.S. Pat. No. 4,034,074.

U.S. Pat. No. 4,098,876.

U.S. Pat. No. 4,235,877.

U.S. Pat. No. 4,376,110.

U.S. Pat. No. 4,429,008.

U.S. Pat. No. 4,436,727.

U.S. Pat. No. 4,452,901.

U.S. Pat. No. 4,489,710.

U.S. Pat. No. 4,507,234.

U.S. Pat. No. 4,554,101.

U.S. Pat. No. 4,569,789.

U.S. Pat. No. 4,603,112.

U.S. Pat. No. 4,625,014.

U.S. Pat. No. 4,638,045.

U.S. Pat. No. 4,671,958.

U.S. Pat. No. 4,673,562.

U.S. Pat. No. 4,683,195.

U.S. Pat. No. 4,699,784.

U.S. Pat. No. 4,735,792.

U.S. Pat. No. 4,751,180.

U.S. Pat. No. 4,769,330.

U.S. Pat. No. 4,777,127.

U.S. Pat. No. 4,866,034.

U.S. Pat. No. 4,873,088.

U.S. Pat. No. 4,877,611.

U.S. Pat. No. 4,897,268.

U.S. Pat. No. 4,912,094.

U.S. Pat. No. 4,935,233.

U.S. Pat. No. 5,017,487.

U.S. Pat. No. 5,075,109.

U.S. Pat. No. 5,145,684.

U.S. Pat. No. 5,151,254.

U.S. Pat. No. 5,215,926.

U.S. Pat. No. 5,240,856.

U.S. Pat. No. 5,350,840.

U.S. Pat. No. 5,359,681.

U.S. Pat. No. 5,399,346.

U.S. Pat. No. 5,399,363.

U.S. Pat. No. 5,466,468.

U.S. Pat. No. 5,472,869.

U.S. Pat. No. 5,543,158.

U.S. Pat. No. 5,552,157.

U.S. Pat. No. 5,565,213.

U.S. Pat. No. 5,567,434.

U.S. Pat. No. 5,633,234.

U.S. Pat. No. 5,641,515.

U.S. Pat. No. 5,738,868.

U.S. Pat. No. 5,741,516.

U.S. Pat. No. 5,795,587.

U.S. Pat. No. 5,811,128.

U.S. Pat. No. 5,814,344.

U.S. Pat. No. 5,820,883.

U.S. Pat. No. 5,853,763.

U.S. Pat. No. 5,874,265.

U.S. Pat. No. 5,928,647.

U.S. Pat. No. 5,942,252.

U.S. Pat. No. 6,110,702.

European Patent No. EP 0,345,242.

Great Britain Patent No. GB 2,200,651.

Intl. Pat. Appl. Publ. No. WO 89/01973.

Intl. Pat. Appl. Publ. No. WO 89/06280.

Intl. Pat. Appl. Publ. No. WO 91/02805.

Intl. Pat. Appl. Publ. No. WO 91/16116.

Intl. Pat. Appl. Publ. No. WO 92/07243.

Intl. Pat. Appl. Publ. No. WO 94/00153.

Intl. Pat. Appl. Publ. No. WO 94/20078.

Intl. Pat. Appl. Publ. No. WO/94/23701.

Intl. Pat. Appl. Publ. No. WO 95/17210.

Intl. Pat. Appl. Publ. No. WO 96/02555.

Intl. Pat. Appl. Publ. No. WO 96/06638.

Intl. Pat. Appl. Publ. No. WO 96/30516.

Intl. Pat. Appl. Publ. No. WO 96/33739.

Intl. Pat. Appl. Publ. No. WO 97/24447.

Intl. Pat. Appl. Publ. No. WO 99/33488.

Aaroston and Todaro, J. Cell. Physiol., 72:141-48, 1968.

Adelman et al., DNA, 2:183, 1983.

Amin et al., Am. J. Pathol., 146:344-56, 1995.

Akaza et al., “Expression of antitumor response. Role of attachment and viability of bacillus Calmette-Guerin to bladder cancer cells,” Cancer, 72:558-63, 1993.

American Type Culture Collection, Catalogue of Cell Lines and Hybridomas, 7th ed., 1992.

Avrameas, “Natural autoantibodies: Self-recognition and physiological autoimmunity,” In: Natural autoantibodies: Their Physiological Role and Regulatory Significance, Shoenfeld and Isenberg (eds.), CRC Press, Boca Raton, Fla., pp. 1-14, 1993.

Azuma et al., “Correlation Between Augmented Resistance to Influenza Virus Infection and Histological Changes in Lung of Mice Treated with Trehalose-6,6′-dimycolate,” Journal of Biological Response Modifiers, 7:473-82, 1988.

Bajorin et al., Proc. Annu. Meet. Am. Soc. Clin. Oncol., 7:A967, 1988.

Baker et al., “Ability of Monophosphoryl Lipid A To Augment the Antibody Response of Young Mice,” Infection and Immunity, 56:3064-66, 1988a.

Baker et al., “Enrichment of Suppressor T Cells by Means of Binding to Monophosphoryl Lipid A,” Infection and Immunity, 58:726-31, 1990.

Baker et al., “Inactivation of Suppressor T-Cell Activity by Nontoxic Monophosphoryl Lipid A,” Infection and Immunity, 56:1076-83, 1988b.

Baker et al., “Molecular structures that influence the immunomodulatory properties of the lipid A and inner core region oligosaccharides of bacterial lipopolysaccharides,” Infection Immunity, 62:2257-69, 1994.

Baker et al., “Structural Features That Influence the Ability of Lipid A and Its Analogs To Abolish Expression of Suppressor T Cell Activity,” Infection and Immunity, 60:2694-701, 1992.

Bakker et al., “Melanocyte lineage-specific antigen gp100 is recognized by melanoma-derived tumor-infiltrating lymphocytes,” J. Exp. Med., 179:1005, 1994.

Banchereau and Steinman, Nature, 392:245-51, 1998.

Banerji et al., “Membrane lipid composition modulates the binding specificity of a monoclonal antibody against liposomes,” Biochim. Biophys. Acta., 689:319-26, 1982.

Barnd et al., “Specific tumor histocompatibility complex-unrestricted recognition of tumor-associated mucins by human cytotoxic T cells,” Proc. Natl. Acad. Sci. USA, 86:7159, 1989.

Barnoud et al., Am. J. Surg. Pathol., 24:830-36, 2000).

Bartlett and Zbar, J. Natl. Cancer Inst., 48:1709, 1972.

Bast et al., “BCG and Cancer,” N. Engl. J. Med., 290:1413-20, 1974.

Bennett et al., “Endogenous Production of Cytotoxic Factors in Serum of BCG-Primed Mice by Monophosphoryl Lipid A, a Detoxified Form of Endotoxin,” Journal of Biological Response Modifiers, 7:65-76, 1988.

Berkner, Biotechniques, 6:616-27, 1988.

Berra et al., Int. J. Cancer, 36:363-66, 1985.

Berra et al., J. Neurochem., 40:777-82, 1983.

Bogoch, “Demonstration of serum precipitin to brain gangliosides,” Nature, 183:392-93, 1960.

Bouchon et al., Biochem. Internatl., 10:531-38, 1985.

Bowen-Pope et al., Proc. Nat'l Acad. Sci. USA, 81:2396-400, 1984.

Bowness et al., “Clostridium perfringens enterotoxin is a superantigen reactive with human T cell receptors V beta 6.9 and V beta 22,” J. Exp. Med., 176:893-96, 1992.

Brade et al., “An Artificial Glycoconjugate Containing the Bisphosphorylated Glucosamine Disaccharide Backbone of Lipid A Binds Lipid A Monoclonal Antibodies,” Infection and Immunity, 61:4514-17, 1993.

Brichard et al., “The tyrosinase gene codes for an antigen recognized by autologous cytolytic T lymphocytes on HLA-A2 melanomas,” J. Exp. Med., 178:489, 1993.

Brodin et al., “Mouse monoclonal antibodies with specificity for the melanoma-associated ganglioside disialyllactosyl ceramide (GD3) also react with the structural analogue disialylparagloboside,” Biochim. Biophys. Acta., 837:349-53, 1985.

Brooks et al., Clin. Exp. Immunol., 39: 477, 1980.

Brown et al., J. Biol. Chem., 255:4980-83, 1980.

Burchell et al., J. Immunol., 131:508-13, 1983.

Bystryn et al., Cancer, 61:1065, 1988.

Cahan et al., “Identification of a human neuroectodermal tumor antigen (OFA-I-2) as ganglioside GD2,” Proc. Natl. Acad. Sci. USA, 79:7629-33, 1982.

Campbell et al., “Intercellular adhesion molecule-1 expression by bladder cancer cells: functional effects,” J. Urol., 151:1385-90, 1994.

Campbell, in Monoclonal Antibody Technology, Laboratory Techniques in Biochemistry and Molecular Biology, Vol. 13, Burden and Von Knippenberg (eds.), Amsterdam, Elseview, pp. 75-83, 1984.

Campbell et al., Int. J. Cancer, 78:182-88, 1998.

Carr and Morrison, “A two-step mechanism for the interaction of Re lipopolysaccharide with erythrocyte membranes,” Rev. Infect. Dis. 6:497-508, 1984.

Carubia et al., Biochem. Biophys. Res. Commun., 120:500-04, 1984.

Chang et al., Crit. Rev. Oncol. Hematol., 22:213, 1996.

Chase et al., “Effect of Monophosphoryl Lipid A on Host Resistance to Bacterial Infection,” Infection and Immunity, 53(3):711-12, 1986.

Chen et al., “Activation of Macrophages From Aging Mice by Detoxified Lipid A,” Journal of Leukocyte Biology, 49:416-22, 1991.

Chen et al., Cancer Res., 54:1065-70, 1994.

Cheng et al., “Bacillus Calmette-Gerin interacts with the carboxyl-terminal heparin bindings domain of fibronectin: implications for BCG-mediated antitumor activity,” J. Urol., 152:1275-80, 1994.

Cheresh and Klier, “Disialoganglioside GD3 distributes preferentially into substrate associated microprocesses on human melanoma cells during their attachment to fibronectin,” J. Cell. Biol., 102:1887-97, 1986.

Cheresh et al., “A monoclonal antibody recognizes an O-acetyl sialic acid in a human melanoma-associated ganglioside,” J. Biol. Chem., 259:7453-59, 1984.

Cheresh et al., “Disialoganglioside GD3 on human melanoma serves as a relevant target antigen for monoclonal antibody-mediated tumor cytolysis,” Proc. Natl. Acad. Sci. USA, 82:5155-59, 1985.

Cheresh et al., “Disialogangliosides GD2 and GD3 are involved in the attachment of human melanoma and neuroblastoma cells to extracellular matrix proteins,” J. Cell. Biol., 102:688-96,1986.

Cheresh et al., “Localization of gangliosides GD2 and GD3 in adhesion plaques and on the surface of human melanoma cells,” Proc. Natl. Acad. Sci. USA, 81:5767-71, 1984.

Cheung et al., “Detection of neuroblastoma cells in bone marrow using GD2 specific monoclonal antibodies,” J. Clin. Oncol., 4:363-69, 1986.

Chu and Sharom, “Gangliosides inhibit T-lymphocyte proliferation by preventing the interaction of interleukin-2 with its cell surface receptors,” Immunology, 79:10-16, 1993.

Cohen, Science, 259:1691-92, 1993.

Colcher et al., Proc. Natl. Acad. Sci. USA, 78:3199, 1987.

Coligan et al., in Current Protocols in Immunology, Vol. 1, Wiley Interscience. Greene (ed.), 1998.

Coombes et al., Vaccine, 14:1429-38, 1996.

Coulie et al., “A new gene coding for a differentiation antigen recognized by autologous cytologic T lymphocytes on HLA-A2 melanomas,” J. Exp. Med., 180:35, 1994.

Deavin et al., Mol. Immunol., 33:145-55, 1996.

DeBruijn et al., Eur. J. Immunol., 21:2963-70, 1991.

Dippold et al., “Immunohistochemical localization of ganglioside GD3 in human malignant melanoma, epithelial tumors and normal tissues,” Cancer Res., 45:3699-705, 1985.

Dippold et al., “Inflammatory response at the tumor site after systemic application of monoclonal anti-GD3-ganglioside antibody to patients with malignant melanoma,” Am. Assoc. Cancer Res., 978:247, 1984.

Dippold et al., Proc. Natl. Acad. Sci. USA, 77:6115, 1980.

Dresser and Phillips, in Immunopotentiation, CIBA Foundation Symposium 18, Elsevier, Amsterdam, p.3, 1973.

Dwivedi et al., “Plasma lipid-bound sialic acid alterations in neoplastic diseases,” Experientia, 46:91-94, 1990.

Elder, “Skin Cancer,” Cancer, 75:245-56, 1995.

Elliott et al., “The D-Galactosamine Loaded Mouse and Its Enhanced Sensitivity to Lipopolysaccharide and Monophosphoryl Lipid A: A Role for Superoxide,” J. Immunol., 10:69-74, 1991.

Euhus et al., Cancer Immunol Immunother., 29:247-54, 1989.

Fawwaz et al., Statutory Invention Registration Patent No. H819, application no. 6-6-5,439, 1990.

Fischer, Handb. Lipid Res., 6:123-234, 1990.

Fisher-Hoch et al., Proc. Nat'l Acad. Sci. USA, 86:317-21, 1989.

Fitzgerald, “Syphilis vaccine: up-regulation of immunogenicity by cyclophosphamide, Ribi adjuvant, and indomethacin confers significant protection against challenge infection in rabbits,” Vaccine, 9:265-72, 1991.

Fleischmann, Park and Hassan, “Fibronectin expression on surgical specimens correlated with the response to intravesical bacillus Calmette-Guerin therapy,” J. Urol., 149:268-71, 1993.

Flexner et al., Ann. N.Y. Acad. Sci., 569:86-103, 1989.

Flexner et al., Vaccine, 8:17-21, 1990.

Foster et al., Cancer Res., 57:3325-30, 1997.

Fraizer et al., Blood, 86:4704-06, 1995.

Fredman et al., Neurol. Res., 8:123-26, 1986.

Freimer et al., “Gangliosides elicit a T-cell independent antibody response,” J. Autoimmun., 6:281-89, 1993.

Freudenberg et al., “ELISA for antibodies to Lipid A, Lipopolyasscharides and other hydrophobic antigens,” Infection, 17:322-24, 1989.

Gaiger et al., Blood, 96:1334, 2000

Garg and Subbarao, “Immune Responses of Systemic and Mucosal Lymphoid Organs to Pnu-Immune Vaccine as a Function of Age and the Efficacy of Monophosphoryl Lipid A as an Adjuvant,” Infection and Immunity, 60:2329-36, 1992.

Gaugler et al., “Human gene MAGE-3 codes for an antigen recognized on a human melanoma by autologous cytolytic T lymphocytes,” J. Exp. Med., 179:921, 1994.

Gefter et al., Somatic Cell Genet., 3:231-36, 1977.

Gennaro et al., Am. J. Ind. Med., 37:275-82, 2000.

Gillard et al., “Antibodies against ganglioside GT₃ in the sera of patients with type I Diabetes mellitus,” J. Immunol., 142:3826-32, 1989.

Gillis, Nature, 268:154-56, 1977.

Glynn, McCoy and Fefer, Cancer Res., 28:434-39, 1968.

Goding, in Monoclonal Antibodies: Principles and Practice, 2d ed., Orlando, Fla., Academic Press, pp. 60-61, 65-66, 71-74, 1986.

Goff et al., Eur. J. Biochem., 130:553-57, 1983.

Grabarek et al., “Endotoxic Lipid A Interaction with Human Platelets,” The Journal of Biological Chemistry, 265:8117-21, 1990.

Graus et al., “Distribution of the ganglioside GD3 in the human nervous system detected by R24 mouse monoclonal antibody,” Brain Res., 324:190-94, 1984.

Guzman et al., Circulation, 88:2838-48, 1993a.

Guzman et al., Cir. Res., 73:1202-07, 1993b.

Hachida et al., Transplant Proc., 22:1663-70, 1990.

Hachida et al., Transplantation, 56:479-82, 1993.

Harada et al., Mol. Urol., 3:357-364, 1999.

Hardings et al., “Effects of pH and polysaccharides on peptide binding to class II major histocompatibility complex molecules,” Proc. Natl. Acad. Sci. USA, 88:2740-44, 1991.

Harel et al., Cancer Res., 50:6311, 1990.

Harlow and Lane, Antibodies: A Laboratory Manual, Cold Spring Harbor Laboratory, 1988.

Helling et al., “Construction of Immunogenic GD₃-conjugate vaccines,” Ann. N. Y. Acad. Sci., 690:396-97, 1993.

Hellstrom et al., “Strong anti-tumor activities of IgG 3 antibodies to a human melanoma-associated ganglioside,” Proc. Natl. Acad. Sci. USA, 82:1499-1502, 1985.

Hirabayashi et al., “Syngeneic monoclonal antibody against melanoma antigen with interspecies cross-reactivity recognize GM₃, a prominent ganglioside of B16 melanoma,” Biol. Chem., 260:13328-33, 1985.

Hirabayashi et al., “Reactivity of mouse monoclonal antibody M2590 against B16 melanoma cells with chemically synthesized GM3 ganglioside,” Biochim. Biophys. Acta, 875:126-28, 1986.

Hirabayashi et al., Jpn. J. Cancer Res., 78:614-20, 1987.

Hoon et al., “Gangliosides from melanoma immunomodulate response of T-cells to interleukin-2,” Cell Immunol., 4:1111-19, 1988.

Horibata and Harris, Exp. Cell. Res., 60:61, 1970.

Horgan, “Total and lipid-bound sialic acid levels in sera from patients with Cancer,” Clin. Chim. Acta., 118:327-31, 1982.

Houghten et al., “Mouse monoclonal IgG3 antibody detecting GD3 ganglioside: A phase I trial in patients with malignant melanoma,” Proc. Natl. Acad. Sci. USA, 82:1242-46, 1985.

Houghton, “Cancer Antigens: Immune Recognition of Self and Altered Self,” J. Exp. Med., 180:1-4, 1994.

Hraba et al., “The Influence of Monophosphoryl Lipid A (MPL™) on Erythrocyte Autoantibody Formation,” Immunobiol., 189:448-56, 1993.

Hunter et al., Vaccine, 9:250: 1991.

Inoue et al., Blood, 88:2267-78, 1996.

Irie and Morton, “Regression of cutaneous metastatic melanoma by intralesional injection with human monoclonal antibody to ganglioside GD2,” Proc. Natl. Acad. Sci. USA, 83:8694-98, 1986.

Irie and Ravindranath, “Gangliosides as targets for monoclonal antibody therapy of cancer,” in Therapeutic monoclonal antibodies, Borrebaeck and Larrick (eds.), Stockton Press, New York, p. 75-94, 1990.

Irie et al., “Human antibody to OFA-I, a tumor antigen, produced in vitro by Epstein-Barr virus transformed human B-lymphoid cell lines,” Proc. Natl. Acad. Sci. USA, 79:5666-70, 1982.

Irie et al., “Melanoma gangliosides and human monoclonal antibody,” in Human Tumor Antigens and Specific Tumor Therapy, Metzgar and Mitchell (eds.), Alan R. Liss, Inc., New York, pp. 115-126, 1989.

Ishioka et al., “MHC interaction and T cell recognition of carbohydrates and glycopeptides,” J. Immunol., 148:2446-51, 1992.

Jackson et al., “Induction of ICAM 1 expression on bladder tumours by BCG immunotherapy,” J. Clin. Pathol., 47:309-12, 1994.

Johnson and Tomai, “A Study of the Cellular and Molecular Mediators of the Adjuvant Action of a Nontoxic Monophosphoryl Lipid A,” Adv. Exp. Med. Biol., 133:567-79, 1988.

Johnson et al., “Characterization of a nontoxic monophosphoryl lipid A,” Rev. Infect. Dis., 9:512-16, 1987.

Johnson et al., “Structural Characterization of Monophosphoryl Lipid A Homologs Obtained from Salmonella minnesota Re595 Lipopolysaccharide,” J. Biol. Chem., 265:8108-16, 1990.

Johnston and Bystryn, “Effect of Cell Wall Skeleton and Monophosphoryl Lipid A Adjuvant on the Immunogenicity of a Murine B16 Melanoma Vaccine,” Journal of the National Cancer Institute, 83:1240-45, 1991.

Jones et al., J Natl Cancer Inst, 66:249-54, 1981.

Kabat et al., “Sequences of Proteins of Immunological Interest,” 5th ed., Public Health Service, National Institutes of Health, Bethesda, Md., pp 647-669, 1991.

Kass-Eisler et al., Proc. Nat'l Acad. Sci. USA, 90:11498-502, 1993.

Katopodis et al., “Lipid-associated sialic acid test for the detection of human cancer,” Cancer Res., 42:5270-75, 1982.

Kawaguchi et al., “Characteristic mode of action of gangliosides in selective modulation of CD4 on human T lymphocytes,” Biochem. Biophys. Res. Commun., 158:1050-55, 1989.

Kawakami et al., “Cloning of the gene coding for a shared human melanoma antigen recognized by autologous T cells infiltrating into tumor,” Proc. Natl. Acad. Sci. USA, 91:3515, 1994.

Kawakami, Eliyahu, Sakaguchi, Robbins, Rivoltini, Yannelli, Appella and Rosenberg, “Identification of the immunodominant peptides of the Mart-1 human melanoma antigen recognized by the majority of HLA-A2 restricted tumor infiltrating lymphocytes,” J. Exp. Med., 180:347-52, 1994.

Kensil et al., J. Am. Vet. Med. Assoc., 199:1423, 1991.

Kloppel et al., “Glycolipid-bound sialic acid in serum: Increased levels in mice and humans bearing mammary carcinomas,” Proc. Natl. Acad. Sci. USA, 74:3011-13, 1977.

Kohler and Milstein, Nature, 256:495-97, 1975.

Kohler and Milstein, Eur. J. Immunol., 6:511-19, 1976.

Kolls et al., Proc. Nat'l Acad. Sci. USA, 91:215-19, 1994.

Koscielak et al., “Glycolipid antigen and its antibody,” Immunochemistry, 5:441-55, 1968.

Kovach et al., “Lipid IV_{A} Inhibits Synthesis and Release of Tumor Necrosis Factor Induced by Lipopolysaccharide in Human Whole Blood Ex Vivo,” J. Exp. Med., 172:77-84, 1990.

Kwok and Higuchi, Nature, 339:237-38, 1989.

Kyogashima et al., Jpn. J. Cancer Res., 78:1229-32, 1987.

Ladisch et al., “Shedding of GD2 ganglioside by human neuroblastoma,” Int. J. Cancer, 39:73-76, 1987.

Lamm et al., “A randomized trial of intravesical doxorubicin and immunotherapy with bacille Calmette-Guérin for transitional-cell carcinoma of the bladder,” N. Engl. J. Med., 325:1205, 1991.

Lengle et al., “Inhibition of the lectin-induced mitogenic response of thymocytes by glycolipids,” Cancer Res., 39:817-922, 1979.

Liepkalns et al., J. Neurochem., 36:1959-65, 1981.

Livingston et al., “The Serologic Response to Meth A Sarcoma Vaccines After Cyclophosphamide Treatment is Additionally Increased by Various Adjuvants,” The Journal of Immunology, 135(2):1505-09, 1985.

Livingston et al., “Approaches to augmenting immunogenicity of the ganglioside GM₂ in mice: purified GM₂ is superior to whole cells,” J. Immunol., 138:1524-29, 1987a.

Livingston et al., “Vaccines containing purified GM₂ gangliosides elicit GM₂ antibodies in melanoma patients,” Proc. Natl. Acad. Sci. USA, 84:2911-15, 1987b.

Ljunggren et al., Nature, 346:476-80, 1990.

Lozzio and Lozzio, Blood, 45:321-34, 1975.

Madonna and Vogel, “Induction of Early-Phase Endotoxin Tolerance in Athymic (Nude) Mice, B-Cell-Deficient (xid) Mice, and Splenectomized Mice,” Infection and Immunity, 53:707-10, 1986.

Mahvi et al., Immunology and cell Biology, 75:456-60, 1997.

Maratea et al., Gene, 40:39-46, 1985.

Masihi et al., “Effects of Nontoxic Lipid A and Endotoxin on Resistance of Mice to Toxoplasma gondii,” Journal of Biological Response Modifiers, 7:535-39, 1988.

Melling et al., J. Immunol., 117:1267-74, 1976.

Menssen et al., J. Cancer Res. Clin. Oncol., 126:226-32, 2000.

Merrifield, J. Am. Chem. Soc., 85:2149-46, 1963.

Miller and Esselman, “Modulation of immune response by antigen reactive lymphocytes after cultivation with gangliosides,” J. Immunol., 115:839-43, 1975.

Minden, “Shared Antigens Between Animal and Human Tumors and Microorganisms,” in BCG in Cancer Immunotherapy, Lamoureux, Turcotte and Portelance (eds.); pp. 73-81, 1976.

Miotti et al., Cancer Res., 65:826, 1985.

Mitchell et al., “Active specific Immunotherapy of melanoma with allogeneic cell lysates: Rationale, results and possible mechanisms of action,” Ann. N.Y. Acad. Sci., 690:153-66, 1993.

Mitchell et al., “Active specific immunotherapy of melanoma: Phase I trial of allogeneic lysates and a novel adjuvant,” Cancer Res., 48:5883-93, 1988.

Mitchell et al., “Active-Specific Immunotherapy for Melanoma,” Journal of Clinical Oncology, 8:856-59, 1990.

Miyake et al., Cancer Res., 48:6154-60, 1988.

Mooney et al., “Bacterial superantigen signaling via HLA class II on human B lymphocytes,” Mol. Immunol., 31:675-81, 1994.

Morrison et al., “Specific ganglioside binding to receptor sites on T lymphocytes that couple to ganglioside-induced decrease of CD4 expression,” Life Sci., 45:1219-24, 1989.

Morton and Ravindranath, in Cancer Medicine, 3rd ed., Holland et al. (eds.), Lea and Febiger, Philadelphia, p.967, 1993.

Morton et al., “Polyvalent Melanoma Vaccine Improves Survival of Patients with Metastatic Melanoma,” John Wayne Cancer Institute at Saint John's Hospital and Health Center, Santa Monica, Calif., reprinted from Specific Immunotherapy of Cancer with Vaccines, Volume 690 of the Annals of the New York Academy of Sciences, 1993.

Morton et al., Ann. Surg., 216:463, 1992.

Morton et al., in Biological Function of Gangliosides, Progress in Brain Research, Vol. 101, pp 251-275, 1994.

Mosmann and Coffman, Ann. Rev. Immunol., 7:145-73, 1989.

Munjal et al., “Combined measurement and significance of lipid-bound sialic acid and carcinoembryonic antigen in detection of human cancer,” Diagn. Immunol., 2:36-43, 1984.

Murphy et al., Proc. Natl. Acad. Sci. USA,83:8258-62, 1986.

Myers et al., “Monophosphoryl Lipid A Behaves as a T-Cell-Independent Type 1 Carrier for Hapten-Specific Antibody Responses in Mice,” Infection and Immunity, 63:168-74, 1995.

Naiki et al., “Properties of antisera to ganglioside GM₁ and AsialoGM₁”, J. Immunol., 113:84-93, 1974.

Nair et al., Nature Biotechnol., 16:364-69, 1998.

Natoli et al., “A murine monoclonal antibody detecting the ganglioside GM2: Characterization of cell surface reactivity,” Cancer Res., 46:4116-20, 1986.

Nonomura et al., Hinyokika Kiyo, 45:593-97, 1999.

Nudelman et al., “Characterization of a human melanoma-associated ganglioside antigen defined by a monoclonal antibody 4.2,” J. Biol. Chem., 257:12752-56, 1982.

Odean et al., “Involvement of Gamma Interferon in Antibody Enhancement by Adjuvants,” Infection and Immunity, 58:427-32, 1990.

Oji et al., Jpn. J. Cancer Res., 90:194-204, 1999.

Old et al., Ann. N. Y. Acad. Sci., 101:80-106, 1962.

Pan et al., Leukemia, 14:1634, 2000.

Parker et al., J. Immunol., 152:163, 1994.

Pascal et al., “Immunochemical studies on normal and Tay-Sachs' brain gangliosides,” Proc. Soc. Exp. Biol. Med., 121:739-43, 1966.

Patek, Collins and Cohn, “Transformed cell lines susceptible or resistant to in vivo surveillance against tumorigenesis,” Nature, 276:510-11, 1978.

Patmasiriwat et al., Leukemia, 13:891-900, 1999.

Paul, Fundamental Immunology, 3rd ed., Raven Press, pp. 243-47, 1993.

Portoukalian et al., “Humoral immune response in disease-free advanced melanoma patients after vaccination with melanoma-associated gangliosides,” Int. J. Cancer, 49:893-99, 1991.

Portoukalian, “Alteration of gangliosides in plasma and red cells of human bearing melanoma tumors,” Biochem. Biophys. Res. Commun., 85:916-20, 1978.

Portoukalian, “Immunoregulatory activity of gangliosides shed by melanoma tumors,” in Gangliosides and Cancer, Oettgen (ed.), New York, VCH Publishers, pp. 207-16, 1989.

Powell and Newman (eds.), “Vaccine Design (the subunit and adjuvant approach),” Plenum Press, NY, 1995.

Prokazova et al., “Sialylated lactosylceramides. Possible inducers of non-specific immunosuppression and atherosclerotic lesions,” Eur.J.Biochem., 171:1-10, 1988.

Pukel et al., “GD3, a prominent ganglioside of human melanoma: Detection and characterization of mouse monoclonal antibody,” J. Exp. Med., 155:1133-47, 1982.

Qureshi et al., “Purification and structural determination of nontoxic lipid A obtained from the Lipopolysaccharide of Salmonella typhimurium,” J. Biol. Chem., 257:11808-15, 1985.

Rabinovich et al., “Vaccine Technologies: View to the Future,” Science, 265:1401-02, 1994.

Raines and Ross, J. Biol. Chem., 257:5154-60, 1982.

Rapport and Graf, “Immunochemical Reactions of Lipids,” Prog. Allergy, 13:273-331, 1969.

Ravindranath and Irie, in Malignant Melanoma: Biology, Diagnosis, and Therapy, Nathanson (ed.), Kluwer Acad., Boston, p. 17, 1988.

Ravindranath and Morton, “Role of gangliosides in active immunotherapy with melanoma vaccine,” Int. Rev. Immunol., 7:303, 1991.

Ravindranath et al., “Human melanoma antigen-acetylated Ganglioside GD₃ is recognized by Cancer antennarius lectin,” J. Biol. Chem., 263:2079-86, 1988.

Ravindranath et al., “An epitope common to gangliosides O-acetyl GD3 and GD3 recognized by antibodies in melanoma patients after active specific immunotherapy,” Cancer Res., 49:3891-97, 1989.

Ravindranath et al., “Ganglioside GM₃:GD₃ Ratio as an Index for the Management of Melanoma,” Cancer, 67:3029-35, 1991.

Ravindranath et al., “Efficacy of tumor cell vaccine after incorporating monophosphoryl lipid A (MPL) in tumor cell membranes containing tumor-associated ganglioside,” Experientia, 50:648-653, 1994a.

Ravindranath et al., “Attachment of Monophosphoryl Lipid A (MPL) to Cells and Liposomes Augments Antibody Response to membrane-bound Gangliosides,” Journal of Autoimmunity, 7:803-16, 1994b.

Ravindranath et al., “Factors affecting the fine specificity and sensitivity of serum antiganglioside antibodies in ELISA,” J. Immunol. Methods, 169:257-72, 1994c.

Real et al., “Class I (unique) tumor antigens of human melanoma. Identification of a 90,000 dalton cell surface glycoprotein by autologous antibody,” J. Exp. Med., 160:1219, 1984.

Reeves et al., Cancer Res., 56:5672-77, 1996.

Reisfeld et al., Melanoma Antigens and Antibodies, p. 317, 1982.

Ribi, “Beneficial modification of the endotoxin molecule,” J. Biol. Resp. Mod., 3:1-9, 1984.

Ribi et al., “Lipid A and immunotherapy,” Rev. Infect. Dis., 6:567-72, 1984.

Ribi et al., “Modulation of humoral and cell mediated immune responses by a structurally established nontoxic lipid A,” in Immunobiology and Immunopharmacology of Bacterial Endotoxins, Szentivanji and Friedman (eds.), Plenum Press, New York, pp. 407-420, 1986.

Rickman et al., Lancet 337:998, 1991.

Rolland, Crit. Rev. Therap. Drug Carrier Systems, 15:143-98, 1998.

Rosenberg et al., Ann. Surg., 210:474, 1989.

Rosenberg et al., N. Engl. J. Med., 319:1676, 1988.

Rosenfeld et al., Science, 252:431-34, 1991.

Rothbard and Taylor, EMBO J., 7:93-100, 1988.

Rott et al., “Protection from experimental allergic encephalomyelitis by application of a bacterial superantigen,” Int. Immunol., 4:347-53, 1992.

Sato et al., “Cytoplasmic membrane-associated protein (CAP) isolated from Streptococcus pyrogenes: as a new bacterial superantigen,” Microbiol. Immunol., 38:139-47, 1994.

Sato et al., Science, 273:352, 1996.

Satoh et al., Pathol. Int., 50:458-71, 2000.

Schuster et al., “Production of antibodies against phosphocholine, phosphatidylcholine, sphingomyelin, and lipid A by injection of liposomes containing lipid A,” J. Immunol., 122:900-05, 1979.

Schwab et al., “Superantigen can reactivate bacterial cell wall-induced arthritis,” J. Immunol., 150:4151-59, 1993.

Shafer and Spitznagel, “Sensitivity of Salmonella typhimurium to polymorphonuclear granulocyte extracts: Role of lipid A,” Rev. Infect. Dis., 6:577-81, 1984.

Shepard et al., J. Clin. Immunol., 11:117-27, 1991.

Sherwin et al., “The production of antisera to gangliosides from human nervous tissue,” Canad. J. Biochem., 42:1640-48, 1964.

Shimizu et al., Int. J. Gynecol. Pathol., 19:158-63,2000.

Shy et al., “Antibodies to GM₁ and GD_{1b} in patients with motor neuron disease with plasma cell dyscrasia,” Ann. Neurol., 25:511-18, 1989.

Siddiqui et al., Cancer Res., 44:5262-65, 1984.

Sidell et al., Cancer Immunol Immunother, 7:151-55, 1979.

Sigma Cell Culture, Volume 9, Number 2, 1993.

Skeiky et al., Infection and Immun., 67:3998-4007, 1999.

Slavin and Strober, Nature, 272:624-26, 1977.

Spinsanti et al., Leuk. Lymphoma, 38:611-19, 2000.

Stiess and Krüger, “Mammalian Cell Culture Media—Overview and Applications,” The Source (Sigma Cell Culture Technical and Product News), 9:1-8, 1993.

Stoute et al. New Engl. J. Med., 336:86-91, 1997.

Svennerholm et al., “Tumor gangliosides as targets for active specific immunotherapy of melanoma in man,” in Biological Function of Gangliosides, Progress in Brain Research, Vol. 101, 1994.

Tai et al., “Ganglioside GM2 as a human tumor antigen (OFA-I-1),” Proc. Natl. Acad. Sci., 80:5392-96, 1983.

Takada et al., “Molecular and Structural Requirements of a Lipoteichoic Acid from Enterococcus hirae ATCC 9790 for Cytokine-Inducing, Antitumor, and Antigenic Activities,” Infection and Immunity, 63:57-65, 1995.

Takahashi et al, J. Immunol., 140:3244, 1988.

Tamaki et al., Leukemia, 13:393-99, 1999.

Tamauchi et al., Immunology, 50:605, 1983.

Tanamoto, “Dissociation of Endotoxic Activities in a Chemically Synthesized Lipid A Precursor after Acetylation,” Infection and Immunity, 63:690-92, 1995.

Tanamoto, “Free Hydroxyl Groups Are Not Required for Endotoxic Activity of Lipid A,” Infection and Immunity, 62:1705-09, 1994a.

Tanamoto, FEBS Lett., 351:325-29, 1994b.

Tautu et al., “Improved procedure for determination of serum lipid-associated sialic acid: Application for early diagnosis of colorectal cancer,” J. Natl. Cancer Inst., 80:1333-37, 1988.

Thor et al., Cancer Res., 46:3118, 1986.

Thurin et al., “Proton NMR and fast-atom bombardment mass spectrometry analysis of the melanoma-associated ganglioside 9-O-acetyl GD3,” J. Biol. Chem., 260:14556-563, 1985.

Timmerman and Levy, Ann. Rev. Med., 50:507-29, 1999.

Tomai and Johnson, “T Cell and Interferon-γ Involvement in the Adjuvant Action of a Detoxified Endotoxin,” Journal of Biological Response Modifiers, 8:625-43, 1989.

Tomai et al., “The Adjuvant Properties of a Nontoxic Monophosphoryl Lipid A in Hyporesponsive and Aging Mice,” Journal of Biological Response Modifiers, 6:99-107, 1987.

Tsuchida et al., J. Dermatol., 11: 129-38, 1984.

Tsuchida et al., “Gangliosides of Human Melanoma: Altered Expression in Vivo and in Vitro,” Cancer Research, 47:1278-81, 1987.

Tsuchida et al., “Gangliosides as tumor markers of human melanoma: bio-chemical and immunologic assays,” in New Horizons of Tumor Immunotherapy, Torisu and Yoshida (eds.), pp. 315-325, 1989.

Tuting et al., J. Immunol., 160:1139-47, 1998.

Ulmer et al., Science, 259:1745-49, 1993.

Vadhan-Raj et al., J. Clin. Oncol., 6:1636, 1988.

van der Bruggen et al., “A gene encoding an antigen recognized by cytolytic T lymphocytes on a human melanoma,” Science, 264:716, 1991.

Verma et al., “Adjuvant Effects of Liposomes Containing Lipid A: Enhancement of Liposomal Antigen Presentation and Recruitment of Macrophages,” Infection and Immunity, 60:2438-44, 1992.

Vijayasaradhi et al., “The melanoma antigen gp75 is the human homologue of the mouse b (brown) locus gene product,” J. Exp. Med., 171:1375, 1990.

Von Bultzingslowen, Pneumologie, 53:266-75, 1999.

Vosika et al., Cancer Immunol. Immunother., 18:107, 1984.

Watson, Ralph, Sarkar and Cohn, “Leukemia viruses associated with mouse myeloma cells,” Proc. Natl. Acad. Sci. USA, 66:344, 1970.

Westrick et al., Biochim. Biophys. Acta, 750:141-48, 1983a.

Westrick et al., Cancer Res., 43:5890-94, 1983b.

Whisler and Yates, “Regulation of lymphocyte responses by human gangliosides. I.

Characteristics of inhibitory effects and the induction of impaired activation,” J. Immuunol., 125:2106-12, 1980.

Wide et al., in Radioimmunoassay Methods, Kirkham and Hunter (eds.), E. and S. Livingstone, Edinburgh, 1970.

Wilschut, “Preparation and properties of phospholipid vesicles,” in Methodologie des liposomes appliquee a la pharmacologie et a la biologies cellulaire, Leserman and Barbet (eds.), INSERM, Paris, pp. 1-10, 1982.

Yamaguchi et al., “Cell-surface antigens of melanoma recognized by human monoclonal antibodies,” Proc. Natl. Acad. Sci. USA, 84:2416-20, 1987.

Yamamoto et al., “In vitro Augmentation of Natural Killer Cell Activity and Production of Interferon-a/β and -γ with Deoxyribonucleic Acid Fraction from Mycobacterium bovis BCG,” Jpn. J. Cancer Res., 79:866-73, 1988.

Yeh et al., “A cell-surface antigen which is present in the ganglioside fraction and shared by human melanomas,” Int. J. Cancer, 29:269-75, 1982.

Yin et al., “Effect of Various Adjuvants on the Antibody Response of Mice to Pneumococcal Polysaccharides,” J. Biol. Resp. Modifiers, 8:190-205, 1989.

Yokoyama et al., “Immunochemical studies with gangliosides,” J. Immunol., 90:372-80, 1963.

Zapata et al., Protein Eng., 8:1057-62, 1995.

Zitvogel et al., Nat. Med., 4:594-600, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below:

What is claimed is:

1. An isolated peptide or polypeptide, comprising at least a first coding region that comprises an amino acid sequence that is at least about 90% identical to the amino acid sequence of any one of SEQ ID NO:669 to SEQ ID NO:2532.

2. The isolated peptide or polypeptide of claim 1, wherein said amino acid sequence is at least about 92% identical to the amino acid sequence of any one of SEQ ID NO:669 to SEQ ID NO:2532.

3. The isolated peptide or polypeptide of claim 2, wherein said amino acid sequence is at least about 94% identical to the amino acid sequence of any one of SEQ ID NO:669 to SEQ ID NO:2532.

4. The isolated peptide or polypeptide of claim 3, wherein said amino acid sequence is at least about 96% identical to the amino acid sequence of any one of SEQ ID NO:669 to SEQ ID NO:2532.

5. The isolated peptide or polypeptide of claim 4, wherein said amino acid sequence is at least about 98% identical to the amino acid sequence of any one of SEQ ID NO:669 to SEQ ID NO:2532.

6. The isolated peptide or polypeptide of claim 4, wherein said amino acid sequence is at least about 99% identical to the amino acid sequence of any one of SEQ ID NO:669 to SEQ ID NO:2532.

7. The isolated peptide or polypeptide of claim 1, wherein said at least a first isolated coding region comprises an at least about 50 contiguous amino acid sequence from any one of SEQ ID NO:669 to SEQ ID NO:2532.

8. The isolated peptide or polypeptide of claim 7, wherein said at least a first isolated coding region comprises an at least about 75 contiguous amino acid sequence from any one of SEQ ID NO:669 to SEQ ID NO:2532.

9. The isolated polypeptide of claim 8, wherein said at least a first isolated coding region comprises an at least about 100 contiguous amino acid sequence from any one of SEQ ID NO:669 to SEQ ID NO:2532.

10. The isolated polypeptide of claim 9, wherein said at least a first isolated coding region comprises an at least about 125 contiguous amino acid sequence from any one of SEQ ID NO:669 to SEQ ID NO:2532.

11. The isolated peptide or polypeptide of claim 1, wherein said at least a first isolated coding region comprises the amino acid sequence of any one of SEQ ID NO:669 to SEQ ID NO:2532.

12. The isolated peptide or polypeptide of claim 1, wherein said at least a first isolated coding region comprises an amino acid sequence that is at least about 93% identical to the amino acid sequence of any one of SEQ ID NO:669 to SEQ ID NO:1380.

13. The isolated peptide or polypeptide of claim 12, wherein said at least a first isolated coding region comprises an amino acid sequence that is at least about 96% identical to the amino acid sequence of any one of SEQ ID NO:669 to SEQ ID NO:1380.

14. The isolated peptide or polypeptide of claim 13, wherein said at least a first isolated coding region comprises an amino acid sequence that is at least about 99% identical to the amino acid sequence of any one of SEQ ID NO:669 to SEQ ID NO:1380.

15. The isolated peptide or polypeptide of claim 1, wherein said at least a first isolated coding region comprises an amino acid sequence that is at least about 93% identical to the amino acid sequence of any one of SEQ ID NO:1381 to SEQ ID NO:1859.

16. The isolated peptide or polypeptide of claim 15, wherein said at least a first isolated coding region comprises an amino acid sequence that is at least about 96% identical to the amino acid sequence of any one of SEQ ID NO:1381 to SEQ ID NO:1859.

17. The isolated peptide or polypeptide of claim 16, wherein said at least a first isolated coding region comprises an amino acid sequence that is at least about 99% identical to the amino acid sequence of any one of SEQ ID NO:1381 to SEQ ID NO:1859.

18. The isolated peptide or polypeptide of claim 1, wherein said at least a first isolated coding region comprises an amino acid sequence that is at least about 93% identical to the amino acid sequence of any one of SEQ ID NO:1860 to SEQ ID NO:2105.

19. The isolated peptide or polypeptide of claim 18, wherein said at least a first isolated coding region comprises an amino acid sequence that is at least about 96% identical to the amino acid sequence of any one of SEQ ID NO:1860 to SEQ ID NO:2105.

20. The isolated peptide or polypeptide of claim 19, wherein said at least a first isolated coding region comprises an amino acid sequence that is at least about 99% identical to the amino acid sequence of any one of SEQ ID NO:1860 to SEQ ID NO:2105.

21. The isolated peptide or polypeptide of claim 1, wherein said at least a first isolated coding region comprises an amino acid sequence that is at least about 93% identical to the amino acid sequence of any one of SEQ ID NO:2106 to SEQ ID NO:2375.

22. The isolated peptide or polypeptide of claim 21, wherein said at least a first isolated coding region comprises an amino acid sequence that is at least about 96% identical to the amino acid sequence of any one of SEQ ID NO:2106 to SEQ ID NO:2375.

23. The isolated peptide or polypeptide of claim 22, wherein said at least a first isolated coding region comprises an amino acid sequence that is at least about 99% identical to the amino acid sequence of any one of SEQ ID NO:2106 to SEQ ID NO:2375.

24. The isolated peptide or polypeptide of claim 1, wherein said at least a first isolated coding region comprises an amino acid sequence that is at least about 93% identical to the amino acid sequence of any one of SEQ ID NO:2376 to SEQ ID NO:2532.

25. The isolated peptide or polypeptide of claim 24, wherein said at least a first isolated coding region comprises an amino acid sequence that is at least about 96% identical to the amino acid sequence of any one of SEQ ID NO:2376 to SEQ ID NO:2532.

26. The isolated peptide or polypeptide of claim 25, wherein said at least a first isolated coding region comprises an amino acid sequence that is at least about 99% identical to the amino acid sequence of any one of SEQ ID NO:2376 to SEQ ID NO:2532.

27. The isolated peptide or polypeptide of claim 1, wherein said at least a first isolated coding region comprises an amino acid sequence selected from the group consisting of SEQ ID NO:669, SEQ ID NO:670, SEQ ID NO:671, SEQ ID NO:672, SEQ ID NO:673, SEQ ID NO:674, SEQ ID NO:675, SEQ ID NO:676, SEQ ID NO:677, SEQ ID NO:678, SEQ ID NO:679, SEQ ID NO:680, SEQ ID NO:681, SEQ ID NO:682, SEQ ID NO:683, SEQ ID NO:684, SEQ ID NO:685, SEQ ID NO:686, SEQ ID NO:687, SEQ ID NO:688, SEQ ID NO:689, SEQ ID NO:690, SEQ ID NO:691, SEQ ID NO:692, SEQ ID NO:693, SEQ ID NO:694, SEQ ID NO:695, SEQ ID NO:696, SEQ ID NO:697, SEQ ID NO:698, SEQ ID NO:699, SEQ ID NO:700, SEQ ID NO:701, SEQ ID NO:702, SEQ ID NO:703, SEQ ID NO:704, SEQ ID NO:705, SEQ ID NO:706, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, SEQ ID NO:719, SEQ ID NO:720, SEQ ID NO:721, SEQ ID NO:722, SEQ ID NO:723, SEQ ID NO:724, SEQ ID NO:725, SEQ ID NO:726, SEQ ID NO:727, SEQ ID NO:728, SEQ ID NO:729, SEQ ID NO:730, SEQ ID NO:731, SEQ ID NO:732, SEQ ID NO:733, SEQ ID NO:734, SEQ ID NO:735, SEQ ID NO:736, SEQ ID NO:737, SEQ ID NO:738, SEQ ID NO:739, SEQ ID NO:740, SEQ ID NO:741, SEQ ID NO:742, SEQ ID NO:743, SEQ ID NO:744, SEQ ID NO:745, SEQ ID NO:746, SEQ ID NO:747, SEQ ID NO:748, SEQ ID NO:749, SEQ ID NO:750, SEQ ID NO:751, SEQ ID NO:752, SEQ ID NO:753, SEQ ID NO:754, SEQ ID NO:755, SEQ ID NO:756, SEQ ID NO:757, SEQ ID NO:758, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:780, SEQ ID NO:781, SEQ ID NO:782, SEQ ID NO:783, SEQ ID NO:784, SEQ ID NO:785, SEQ ID NO:786, SEQ ID NO:787, SEQ ID NO:788, SEQ ID NO:789, SEQ ID NO:790, SEQ ID NO:791, SEQ ID NO:792, SEQ ID NO:793, SEQ ID NO:794, SEQ ID NO:795, SEQ ID NO:796, SEQ ID NO:797, SEQ ID NO:798, SEQ ID NO:799, SEQ ID NO:800, SEQ ID NO:801, SEQ ID NO:802, SEQ ID NO:803, SEQ ID NO:804, SEQ ID NO:805, SEQ ID NO:806, SEQ ID NO:807, SEQ ID NO:808, SEQ ID NO:809, SEQ ID NO:810, SEQ ID NO:811, SEQ ID NO:812, SEQ ID NO:813, SEQ ID NO:814, SEQ ID NO:815, SEQ ID NO:816, SEQ ID NO:817, SEQ ID NO:818, SEQ ID NO:819, SEQ ID NO:820, SEQ ID NO:821, SEQ ID NO:822, SEQ ID NO:823, SEQ ID NO:824, SEQ ID NO:825, SEQ ID NO:826, SEQ ID NO:827, SEQ ID NO:828, SEQ ID NO:829, SEQ ID NO:830, SEQ ID NO:831, SEQ ID NO:832, SEQ ID NO:833, SEQ ID NO:834, SEQ ID NO:835, SEQ ID NO:836, SEQ ID NO:837, SEQ ID NO:838, SEQ ID NO:839, SEQ ID NO:840, SEQ ID NO:841, SEQ ID NO:842, SEQ ID NO:843, SEQ ID NO:844, SEQ ID NO:845, SEQ ID NO:846, SEQ ID NO:847, SEQ ID NO:848, SEQ ID NO:849, SEQ ID NO:850, SEQ ID NO:851, SEQ ID NO:852, SEQ ID NO:853, SEQ ID NO:854, SEQ ID NO:855, SEQ ID NO:856, SEQ ID NO:857, SEQ ID NO:858, SEQ ID NO:859, SEQ ID NO:860, SEQ ID NO:861, SEQ ID NO:862, SEQ ID NO:863, SEQ ID NO:864, SEQ ID NO:865, SEQ ID NO:866, SEQ ID NO:867, SEQ ID NO:868, SEQ ID NO:869, SEQ ID NO:870, SEQ ID NO:871, SEQ ID NO:872, SEQ ID NO:873, SEQ ID NO:874, SEQ ID NO:875, SEQ ID NO:876, SEQ ID NO:877, SEQ ID NO:878, SEQ ID NO:879, SEQ ID NO:880, SEQ ID NO:881, SEQ ID NO:882, SEQ ID NO:883, SEQ ID NO:884, SEQ ID NO:885, SEQ ID NO:886, SEQ ID NO:887, SEQ ID NO:888, SEQ ID NO:889, SEQ ID NO:890, SEQ ID NO:891, SEQ ID NO:892, SEQ ID NO:893, SEQ ID NO:894, SEQ ID NO:895, SEQ ID NO:896, SEQ ID NO:897, SEQ ID NO:898, SEQ ID NO:899, SEQ ID NO:900, SEQ ID NO:901, SEQ ID NO:902, SEQ ID NO:903, SEQ ID NO:904, SEQ ID NO:905, SEQ ID NO:906, SEQ ID NO:907, SEQ ID NO:908, SEQ ID NO:909, SEQ ID NO:910, SEQ ID NO:911, SEQ ID NO:912, SEQ ID NO:913, SEQ ID NO:914, SEQ ID NO:915, SEQ ID NO:916, SEQ ID NO:917, SEQ ID NO:918, SEQ ID NO:919, SEQ ID NO:920, SEQ ID NO:921, SEQ ID NO:922, SEQ ID NO:923, SEQ ID NO:924, SEQ ID NO:925, SEQ ID NO:926, SEQ ID NO:927, SEQ ID NO:928, SEQ ID NO:929, SEQ ID NO:930, SEQ ID NO:931, SEQ ID NO:932, SEQ ID NO:933, SEQ ID NO:934, SEQ ID NO:935, SEQ ID NO:936, SEQ ID NO:937, SEQ ID NO:938, SEQ ID NO:939, SEQ ID NO:940, SEQ ID NO:941, SEQ ID NO:942, SEQ ID NO:943, SEQ ID NO:944, SEQ ID NO:945, SEQ ID NO:946, SEQ ID NO:947, SEQ ID NO:948, SEQ ID NO:949, SEQ ID NO:950, SEQ ID NO:951, SEQ ID NO:952, SEQ ID NO:953, SEQ ID NO:954, SEQ ID NO:955, SEQ ID NO:956, SEQ ID NO:957, SEQ ID NO:958, SEQ ID NO:959, SEQ ID NO:960, SEQ ID NO:961, SEQ ID NO:962, SEQ ID NO:963, SEQ ID NO:964, SEQ ID NO:965, SEQ ID NO:966, SEQ ID NO:967, SEQ ID NO:968, SEQ ID NO:969, SEQ ID NO:970, SEQ ID NO:971, SEQ ID NO:972, SEQ ID NO:973, SEQ ID NO:974, SEQ ID NO:975, SEQ ID NO:976, SEQ ID NO:977, SEQ ID NO:978, SEQ ID NO:979, SEQ ID NO:980, SEQ ID NO:981, SEQ ID NO:982, SEQ ID NO:983, SEQ ID NO:984, SEQ ID NO:985, SEQ ID NO:986, SEQ ID NO:987, SEQ ID NO:988, SEQ ID NO:989, SEQ ID NO:990, SEQ ID NO:991, SEQ ID NO:992, SEQ ID NO:993, SEQ ID NO:994, SEQ ID NO:995, SEQ ID NO:996, SEQ ID NO:997, SEQ ID NO:998, SEQ ID NO:999, SEQ ID NO:1000, SEQ ID NO:1001, SEQ ID NO:1002, SEQ ID NO:1003, SEQ ID NO:1004, SEQ ID NO:1005, SEQ ID NO:1006, SEQ ID NO:1007, SEQ ID NO:1008, SEQ ID NO:1009, SEQ ID NO:1010, SEQ ID NO:1011, SEQ ID NO:1012, SEQ ID NO:1013, SEQ ID NO:1014, SEQ ID NO:1015, SEQ ID NO:1016, SEQ ID NO:1017, SEQ ID NO:1018, SEQ ID NO:1019, SEQ ID NO:1020, SEQ ID NO:1021, SEQ ID NO:1022, SEQ ID NO:1023, SEQ ID NO:1024, SEQ ID NO:1025, SEQ ID NO:1026, SEQ ID NO:1027, SEQ ID NO:1028, SEQ ID NO:1029, SEQ ID NO:1030, SEQ ID NO:1031, SEQ ID NO:1032, SEQ ID NO:1033, SEQ ID NO:1034, SEQ ID NO:1035, SEQ ID NO:1036, SEQ ID NO:1037, SEQ ID NO:1038, SEQ ID NO:1039, SEQ ID NO:1040, SEQ ID NO:1041, SEQ ID NO:1042, SEQ ID NO:1043, SEQ ID NO:1044, SEQ ID NO:1045, SEQ ID NO:1046, SEQ ID NO:1047, SEQ ID NO:1048, SEQ ID NO:1049, SEQ ID NO:1050, SEQ ID NO:1051, SEQ ID NO:1052, SEQ ID NO:1053, SEQ ID NO:1054, SEQ ID NO:1055, SEQ ID NO:1056, SEQ ID NO:1057, SEQ ID NO:1058, SEQ ID NO:1059, SEQ ID NO:1060, SEQ ID NO:1061, SEQ ID NO:1062, SEQ ID NO:1063, SEQ ID NO:1064, SEQ ID NO:1065, SEQ ID NO:1066, SEQ ID NO:1067, SEQ ID NO:1068, SEQ ID NO:1069, SEQ ID NO:1070, SEQ ID NO:1071, SEQ ID NO:1072, SEQ ID NO:1073, SEQ ID NO:1074, SEQ ID NO:1075, SEQ ID NO:1076, SEQ ID NO:1077, SEQ ID NO:1078, SEQ ID NO:1079, SEQ ID NO:1080, SEQ ID NO:1081, SEQ ID NO:1082, SEQ ID NO:1083, SEQ ID NO:1084, SEQ ID NO:1085, SEQ ID NO:1086, SEQ ID NO:1087, SEQ ID NO:1088, SEQ ID NO:1089, SEQ ID NO:1090, SEQ ID NO:1091, SEQ ID NO:1092, SEQ ID NO:1093, SEQ ID NO:1094, SEQ ID NO:1095, SEQ ID NO:1096, SEQ ID NO:1097, SEQ ID NO:1098, SEQ ID NO:1099, SEQ ID NO:1100, SEQ ID NO:1101, SEQ ID NO:1102, SEQ ID NO:1103, SEQ ID NO:1104, SEQ ID NO:1105, SEQ ID NO:1106, SEQ ID NO:1107, SEQ ID NO:1108, SEQ ID NO:1109, SEQ ID NO:1110, SEQ ID NO:1111, SEQ ID NO:1112, SEQ ID NO:1113, SEQ ID NO:1114, SEQ ID NO:1115, SEQ ID NO:1116, SEQ ID NO:1117, SEQ ID NO:1118, SEQ ID NO:1119, SEQ ID NO:1120, SEQ ID NO:1121, SEQ ID NO:1122, SEQ ID NO:1123, SEQ ID NO:1124, SEQ ID NO:1125, SEQ ID NO:1126, SEQ ID NO:1127, SEQ ID NO:1128, SEQ ID NO:1129, SEQ ID NO:1130,SEQ ID NO:1131,SEQ ID NO:1132, SEQ ID NO:1133, SEQ ID NO:1134, SEQ ID NO:1135, SEQ ID NO:1136, SEQ ID NO:1137, SEQ ID NO:1138, SEQ ID NO:1139, SEQ ID NO:1140, SEQ ID NO:1141, SEQ ID NO:1142, SEQ ID NO:1143, SEQ ID NO:1144, SEQ ID NO:1145, SEQ ID NO:1146, SEQ ID NO:1147, SEQ ID NO:1148, SEQ ID NO:1149, SEQ ID NO:1150, SEQ ID NO:1151, SEQ ID NO:1152, SEQ ID NO:1153, SEQ ID NO:1154, SEQ ID NO:1155, SEQ ID NO:1156, SEQ ID NO:1157, SEQ ID NO:1158, SEQ ID NO:1159, SEQ ID NO:1160, SEQ ID NO:1161, SEQ ID NO:1162, SEQ ID NO:1163, SEQ ID NO:1164, SEQ ID NO:1165, SEQ ID NO:1166, SEQ ID NO:1167, SEQ ID NO:1168, SEQ ID NO:1169, SEQ ID NO:1170, SEQ ID NO:1171, SEQ ID NO:1172, SEQ ID NO:1173, SEQ ID NO:1174, SEQ ID NO:1175, SEQ ID NO:1176, SEQ ID NO:1177, SEQ ID NO:11711, SEQ ID NO:1179, SEQ ID NO:1180, SEQ ID NO:1181, SEQ ID NO:1182, SEQ ID NO:1183, SEQ ID NO:1184, SEQ ID NO:1185, SEQ ID NO:1186, SEQ ID NO:1187, SEQ ID NO:1188, SEQ ID NO:1189, SEQ ID NO:1190, SEQ ID NO:1191, SEQ ID NO:1192, SEQ ID NO:1193, SEQ ID NO:1194, SEQ ID NO:1195, SEQ ID NO:1196, SEQ ID NO:1197, SEQ ID NO:1198, SEQ ID NO:1199, SEQ ID NO:1200, SEQ ID NO:1201, SEQ ID NO:1202, SEQ ID NO:1203, SEQ ID NO:1204, SEQ ID NO:1205, SEQ ID NO:1206, SEQ ID NO:1207, SEQ ID NO:1208, SEQ ID NO:1209, SEQ ID NO:1210, SEQ ID NO:1211, SEQ ID NO:1212, SEQ ID NO:1213, SEQ ID NO:1214, SEQ ID NO:1215, SEQ ID NO:1216, SEQ ID NO:1217, SEQ ID NO:1218, SEQ ID NO:1219, SEQ ID NO:1220, SEQ ID NO:1221, SEQ ID NO:1222, SEQ ID NO:1223, SEQ ID NO:1224, SEQ ID NO:1225, SEQ ID NO:1226, SEQ ID NO:1227, SEQ ID NO:1228, SEQ ID NO:1229, SEQ ID NO:1230, SEQ ID NO:1231, SEQ ID NO:1232, SEQ ID NO:1233, SEQ ID NO:1234, SEQ ID NO:1235, SEQ ID NO:1236, SEQ ID NO:1237, SEQ ID NO:1238, SEQ ID NO:1239, SEQ ID NO:1240, SEQ ID NO:1241, SEQ ID NO:1242, SEQ ID NO:1243, SEQ ID NO:1244, SEQ ID NO:1245, SEQ ID NO:1246, SEQ ID NO:1247, SEQ ID NO:1248, SEQ ID NO:1249, SEQ ID NO:1250, SEQ ID NO:1251, SEQ ID NO:1252, SEQ ID NO:1253, SEQ ID NO:1254, SEQ ID NO:1255, SEQ ID NO:1256, SEQ ID NO:1257, SEQ ID NO:1258, SEQ ID NO:1259, SEQ ID NO:1260, SEQ ID NO:1261, SEQ ID NO:1262, SEQ ID NO:1263, SEQ ID NO:1264, SEQ ID NO:1265, SEQ ID NO:1266, SEQ ID NO:1267, SEQ ID NO:1268, SEQ ID NO:1269, SEQ ID NO:1270, SEQ ID NO:1271, SEQ ID NO:1272, SEQ ID NO:1273, SEQ ID NO:1274, SEQ ID NO:1275, SEQ ID NO:1276, SEQ ID NO:1277, SEQ ID NO:1278, SEQ ID NO:1279, SEQ ID NO:1280, SEQ ID NO:1281, SEQ ID NO:1282, SEQ ID NO:1283, SEQ ID NO:1284, SEQ ID NO:1285, SEQ ID NO:1286, SEQ ID NO:1287, SEQ ID NO:1288, SEQ ID NO:1289, SEQ ID NO:1290, SEQ ID NO:1291, SEQ ID NO:1292, SEQ ID NO:1293, SEQ ID NO:1294, SEQ ID NO:1295, SEQ ID NO:1296, SEQ ID NO:1297, SEQ ID NO:1298, SEQ ID NO:1299, SEQ ID NO:1300, SEQ ID NO:1301, SEQ ID NO:1302, SEQ ID NO:1303, SEQ ID NO:1304, SEQ ID NO:1305, SEQ ID NO:1306, SEQ ID NO:1307, SEQ ID NO:1308, SEQ ID NO:1309, SEQ ID NO:1310, SEQ ID NO:1311, SEQ ID NO:1312, SEQ ID NO:1313, SEQ ID NO:1314, SEQ ID NO:1315, SEQ ID NO:1316, SEQ ID NO:1317, SEQ ID NO:1318, SEQ ID NO:1319, SEQ ID NO:1320, SEQ ID NO:1321, SEQ ID NO:1322, SEQ ID NO:1323, SEQ ID NO:1324, SEQ ID NO:1325, SEQ ID NO:1326, SEQ ID NO:1327, SEQ ID NO:1328, SEQ ID NO:1329, SEQ ID NO:1330, SEQ ID NO:1331, SEQ ID NO:1332, SEQ ID NO:1333, SEQ ID NO:1334, SEQ ID NO:1335, SEQ ID NO:1336, SEQ ID NO:1337, SEQ ID NO:1338, SEQ ID NO:1339, SEQ ID NO:1340, SEQ ID NO:1341, SEQ ID NO:1342, SEQ ID NO:1343, SEQ ID NO:1344, SEQ ID NO:1345, SEQ ID NO:1346, SEQ ID NO:1347, SEQ ID NO:1348, SEQ ID NO:1349, SEQ ID NO:1350, SEQ ID NO:1351, SEQ ID NO:1352, SEQ ID NO:1353, SEQ ID NO:1354, SEQ ID NO:1355, SEQ ID NO:1356, SEQ ID NO:1357, SEQ ID NO:1358, SEQ ID NO:1359, SEQ ID NO:1360, SEQ ID NO:1361, SEQ ID NO:1362, SEQ ID NO:1363, SEQ ID NO:1364, SEQ ID NO:1365, SEQ ID NO:1366, SEQ ID NO:1367, SEQ ID NO:1368, SEQ ID NO:1369, SEQ ID NO:1370, SEQ ID NO:1371, SEQ ID NO:1372, SEQ ID NO:1373, SEQ ID NO:1374, SEQ ID NO:1375, SEQ ID NO:1376, SEQ ID NO:1377, SEQ ID NO:1378, SEQ ID NO:1379, and SEQ ID NO:1380.

28. The isolated peptide or polypeptide of claim 1, wherein said at least a first isolated coding region comprises an amino acid sequence selected from the group consisting of SEQ ID NO:1381, SEQ ID NO:1382, SEQ ID NO:1383, SEQ ID NO:1384, SEQ ID NO:1385, SEQ ID NO:1386, SEQ ID NO:1387, SEQ ID NO:1388, SEQ ID NO:1389, SEQ ID NO:1390, SEQ ID NO:1391, SEQ ID NO:1392, SEQ ID NO:1393, SEQ ID NO:1394, SEQ ID NO:1395, SEQ ID NO:1396, SEQ ID NO:1397, SEQ ID NO:1398, SEQ ID NO:1399, SEQ ID NO:1400, SEQ ID NO:1401, SEQ ID NO:1402, SEQ ID NO:1403, SEQ ID NO:1404, SEQ ID NO:1405, SEQ ID NO:1406, SEQ ID NO:1407, SEQ ID NO:1408, SEQ ID NO:1409, SEQ ID NO:1410, SEQ ID NO:1411, SEQ ID NO:1412, SEQ ID NO:1413, SEQ ID NO:1414, SEQ ID NO:1415, SEQ ID NO:1416, SEQ ID NO:1417, SEQ ID NO:1418, SEQ ID NO:1419, SEQ ID NO:1420, SEQ ID NO:1421, SEQ ID NO:1422, SEQ ID NO:1423, SEQ ID NO:1424, SEQ ID NO:1425, SEQ ID NO:1426, SEQ ID NO:1427, SEQ ID NO:1428, SEQ ID NO:1429, SEQ ID NO:1430, SEQ ID NO:1431, SEQ ID NO:1432, SEQ ID NO:1433, SEQ ID NO:1434, SEQ ID NO:1435, SEQ ID NO:1436, SEQ ID NO:1437, SEQ ID NO:1438, SEQ ID NO:1439, SEQ ID NO:1440, SEQ ID NO:1441, SEQ ID NO:1442, SEQ ID NO:1443, SEQ ID NO:1444, SEQ ID NO:1445, SEQ ID NO:1446, SEQ ID NO:1447, SEQ ID NO:1448, SEQ ID NO:1449, SEQ ID NO:1450, SEQ ID NO:1451, SEQ ID NO:1452, SEQ ID NO:1453, SEQ ID NO:1454, SEQ ID NO:1455, SEQ ID NO:1456, SEQ ID NO:1457, SEQ ID NO:1458, SEQ ID NO:1459, SEQ ID NO:1460, SEQ ID NO:1461, SEQ ID NO:1462, SEQ ID NO:1463, SEQ ID NO:1464, SEQ ID NO:1465, SEQ ID NO:1466, SEQ ID NO:1467, SEQ ID NO:1468, SEQ ID NO:1469, SEQ ID NO:1470, SEQ ID NO:1471, SEQ ID NO:1472, SEQ ID NO:1473, SEQ ID NO:1474, SEQ ID NO:1475, SEQ ID NO:1476, SEQ ID NO:1477, SEQ ID NO:1478, SEQ ID NO:1479, SEQ ID NO:1480, SEQ ID NO:1481, SEQ ID NO:1482, SEQ ID NO:1483, SEQ ID NO:1484, SEQ ID NO:1485, SEQ ID NO:1486, SEQ ID NO:1487, SEQ ID NO:1488, SEQ ID NO:1489, SEQ ID NO:1490, SEQ ID NO:1491, SEQ ID NO:1492, SEQ ID NO:1493, SEQ ID NO:1494, SEQ ID NO:1495, SEQ ID NO:1496, SEQ ID NO:1497, SEQ ID NO:1498, SEQ ID NO:1499, SEQ ID NO:1500, SEQ ID NO:1501, SEQ ID NO:1502, SEQ ID NO:1503, SEQ ID NO:1504, SEQ ID NO:1505, SEQ ID NO:1506, SEQ ID NO:1507, SEQ ID NO:1508, SEQ ID NO:1509, SEQ ID NO:1510, SEQ ID NO:1511, SEQ ID NO:1512, SEQ ID NO:1513, SEQ ID NO:1514, SEQ ID NO:1515, SEQ ID NO:1516, SEQ ID NO:1517, SEQ ID NO:1518, SEQ ID NO:1519, SEQ ID NO:1520, SEQ ID NO:1521, SEQ ID NO:1522, SEQ ID NO:1523, SEQ ID NO:1524, SEQ ID NO:1525, SEQ ID NO:1526, SEQ ID NO:1527, SEQ ID NO:1528, SEQ ID NO:1529, SEQ ID NO:1530, SEQ ID NO:1531, SEQ ID NO:1532, SEQ ID NO:1533, SEQ ID NO:1534, SEQ ID NO:1535, SEQ ID NO:1536, SEQ ID NO:1537, SEQ ID NO: NO:1538, SEQ ID NO:1539, SEQ ID NO:1540, SEQ ID NO:1541, SEQ ID NO:1542, SEQ ID NO:1543, SEQ ID NO:1544, SEQ ID NO:1545, SEQ ID NO:1546, SEQ ID NO:1547, SEQ ID NO:1548, SEQ ID NO:1549, SEQ ID NO:1550, SEQ ID NO:1551, SEQ ID NO:1552, SEQ ID NO:1553, SEQ ID NO:1554, SEQ ID NO:1555, SEQ ID NO:1556, SEQ ID NO:1557, SEQ ID NO:1558, SEQ ID NO:1559, SEQ ID NO:1560, SEQ ID NO:1561, SEQ ID NO:1562, SEQ ID NO:1563, SEQ ID NO:1564, SEQ ID NO:1565, SEQ ID NO:1566, SEQ ID NO:1567, SEQ ID NO:1568, SEQ ID NO:1569, SEQ ID NO:1570, SEQ ID NO:1571, SEQ ID NO:1572, SEQ ID NO:1573, SEQ ID NO:1574, SEQ ID NO:1575, SEQ ID NO:1576, SEQ ID NO:1577, SEQ ID NO:1578, SEQ ID NO:1579, SEQ ID NO:1580, SEQ ID NO:1581, SEQ ID NO:1582, SEQ ID NO:1583, SEQ ID NO:1584, SEQ ID NO:1585, SEQ ID NO:1586, SEQ ID NO:1587, SEQ ID NO:1588, SEQ ID NO:1589, SEQ ID NO:1590, SEQ ID NO:1591, SEQ ID NO:1592, SEQ ID NO:1593, SEQ ID NO:1594, SEQ ID NO:1595, SEQ ID NO:1596, SEQ ID NO:1597, SEQ ID NO:1598, SEQ ID NO:1599, SEQ ID NO:1600, SEQ ID NO:1601, SEQ ID NO:1602, SEQ ID NO:1603, SEQ ID NO:1604, SEQ ID NO:1605, SEQ ID NO:1606, SEQ ID NO:1607, SEQ ID NO:1608, SEQ ID NO:1609, SEQ ID NO:1610, SEQ ID NO:1611, SEQ ID NO:1612, SEQ ID NO:1613, SEQ ID NO:1614, SEQ ID NO:1615, SEQ ID NO:1616, SEQ ID NO:1617, SEQ ID NO:1618, SEQ ID NO:1619, SEQ ID NO:1620, SEQ ID NO:1621, SEQ ID NO:1622, SEQ ID NO:1623, SEQ ID NO:1624, SEQ ID NO:1625, SEQ ID NO:1626, SEQ ID NO:1627, SEQ ID NO:1628, SEQ ID NO:1629, SEQ ID NO:1630, SEQ ID NO:1631, SEQ ID NO:1632, SEQ ID NO:1633, SEQ ID NO:1634, SEQ ID NO:1635, SEQ ID NO:1636, SEQ ID NO:1637, SEQ ID NO:1638, SEQ ID NO:1639, SEQ ID NO:1640, SEQ ID NO:1641, SEQ ID NO:1642, SEQ ID NO:1643, SEQ ID NO:1644, SEQ ID NO:1645, SEQ ID NO:1646, SEQ ID NO:1647, SEQ ID NO:1648, SEQ ID NO:1649, SEQ ID NO:1650, SEQ ID NO:1651, SEQ ID NO:1652, SEQ ID NO:1653, SEQ ID NO:1654, SEQ ID NO:1655, SEQ ID NO:1656, SEQ ID NO:1657, SEQ ID NO:1658, SEQ ID NO:1659, SEQ ID NO:1660, SEQ ID NO:1661, SEQ ID NO:1662, SEQ ID NO:1663, SEQ ID NO:1664, SEQ ID NO:1665, SEQ ID NO:1666, SEQ ID NO:1667, SEQ ID NO:1668, SEQ ID NO:1669, SEQ ID NO:1670, SEQ ID NO:1671, SEQ ID NO:1672, SEQ ID NO:1673, SEQ ID NO:1674, SEQ ID NO:1675, SEQ ID NO:1676, SEQ ID NO:1677, SEQ ID NO:1678, SEQ ID NO:1679, SEQ ID NO:1680, SEQ ID NO:1681, SEQ ID NO:1682, SEQ ID NO:1683, SEQ ID NO:1684, SEQ ID NO:1685, SEQ ID NO:1686, SEQ ID NO:1687, SEQ ID NO:1688, SEQ ID NO:1689, SEQ ID NO:1690, SEQ ID NO:1691, SEQ ID NO:1692, SEQ ID NO:1693, SEQ ID NO:1694, SEQ ID NO:1695, SEQ ID NO:1696, SEQ ID NO:1697, SEQ ID NO:1698, SEQ ID NO:1699, SEQ ID NO:1700, SEQ ID NO:1701, SEQ ID NO:1702, SEQ ID NO:1703, SEQ ID NO:1704, SEQ ID NO:1705, SEQ ID NO:1706, SEQ ID NO:1707, SEQ ID NO:1708, SEQ ID NO:1709, SEQ ID NO:1710, SEQ ID NO:1711, SEQ ID NO:1712, SEQ ID NO:1713, SEQ ID NO:1714, SEQ ID NO:1715, SEQ ID NO:1716, SEQ ID NO:1717, SEQ ID NO:1718, SEQ ID NO:1719, SEQ ID NO:1720, SEQ ID NO:1721, SEQ ID NO:1722, SEQ ID NO:1723, SEQ ID NO:1724, SEQ ID NO:1725, SEQ ID NO:1726, SEQ ID NO:1727, SEQ ID NO:1728, SEQ ID NO:1729, SEQ ID NO:1730, SEQ ID NO:1731, SEQ ID NO:1732, SEQ ID NO:1733, SEQ ID NO:1734, SEQ ID NO:1735, SEQ ID NO:1736, SEQ ID NO:1737, SEQ ID NO:1738, SEQ ID NO:1739, SEQ ID NO:1740, SEQ ID NO:1741, SEQ ID NO:1742, SEQ ID NO:1743, SEQ ID NO:1744, SEQ ID NO:1745, SEQ ID NO:1746, SEQ ID NO:1747, SEQ ID NO:1748, SEQ ID NO:1749, SEQ ID NO:1750, SEQ ID NO:1751, SEQ ID NO:1752, SEQ ID NO:1753, SEQ ID NO:1754, SEQ ID NO:1755, SEQ ID NO:1756, SEQ ID NO:1757, SEQ ID NO:1758, SEQ ID NO:1759, SEQ ID NO:1760, SEQ ID NO:1761, SEQ ID NO:1762, SEQ ID NO:1763, SEQ ID NO:1764, SEQ ID NO:1765, SEQ ID NO:1766, SEQ ID NO:1767, SEQ ID NO:1768, SEQ ID NO:1769, SEQ ID NO:1770, SEQ ID NO:1771, SEQ ID NO:1772, SEQ ID NO:1773, SEQ ID NO:1774, SEQ ID NO:1775, SEQ ID NO:1776, SEQ ID NO:1777, SEQ ID NO:1778, SEQ ID NO:1779, SEQ ID NO:1780, SEQ ID NO:1781, SEQ ID NO:1782, SEQ ID NO:1783, SEQ ID NO:1784, SEQ ID NO:1785, SEQ ID NO:1786, SEQ ID NO:1787, SEQ ID NO:1788, SEQ ID NO:1789, SEQ ID NO:1790, SEQ ID NO:1791, SEQ ID NO:1792, SEQ ID NO:1793, SEQ ID NO:1794, SEQ ID NO:1795, SEQ ID NO:1796, SEQ ID NO:1797, SEQ ID NO:1798, SEQ ID NO:1799, SEQ ID NO:1800, SEQ ID NO:1801, SEQ ID NO:1802, SEQ ID NO:1803, SEQ ID NO:1804, SEQ ID NO:1805, SEQ ID NO:1806, SEQ ID NO:1807, SEQ ID NO:1808, SEQ ID NO:1809, SEQ ID NO:1810, SEQ ID NO:1811, SEQ ID NO:1812, SEQ ID NO:1813, SEQ ID NO:1814, SEQ ID NO:1815, SEQ ID NO:1816, SEQ ID NO:1817, SEQ ID NO:1818, SEQ ID NO:1819, SEQ ID NO:1820, SEQ ID NO:1821, SEQ ID NO:1822, SEQ ID NO:1823, SEQ ID NO:1824, SEQ ID NO:1825, SEQ ID NO:1826, SEQ ID NO:1827, SEQ ID NO:1828, SEQ ID NO:1829, SEQ ID NO:1830, SEQ ID NO:1831, SEQ ID NO:1832, SEQ ID NO:1833, SEQ ID NO:1834, SEQ ID NO:1835, SEQ ID NO:1836, SEQ ID NO:1837, SEQ ID NO:1838, SEQ ID NO:1839, SEQ ID NO:1840, SEQ ID NO:1841, SEQ ID NO:1842, SEQ ID NO:1843, SEQ ID NO:1844, SEQ ID NO:1845, SEQ ID NO:1846, SEQ ID NO:1847, SEQ ID NO:1848, SEQ ID NO:1849, SEQ ID NO:1850, SEQ ID NO:1851, SEQ ID NO:1852, SEQ ID NO:1853, SEQ ID NO:1854, SEQ ID NO:1855, SEQ ID NO:1856, SEQ ID NO:1857, SEQ ID NO:1858, and SEQ ID NO:1859.

29. The isolated peptide or polypeptide of claim 1, wherein said at least a first isolated coding region comprises an amino acid sequence selected from the group consisting of SEQ ID NO:1860, SEQ ID NO:1861, SEQ ID NO:1862, SEQ ID NO:1863, SEQ ID NO:1864, SEQ ID NO:1865, SEQ ID NO:1866, SEQ ID NO:1867, SEQ ID NO:1868, SEQ ID NO:1869, SEQ ID NO:1870, SEQ ID NO:1871, SEQ ID NO:1872, SEQ ID NO:1873, SEQ ID NO:1874, SEQ ID NO:1875, SEQ ID NO:1876, SEQ ID NO:1877, SEQ ID NO:1878, SEQ ID NO:1879, SEQ ID NO:1880, SEQ ID NO:1881, SEQ ID NO:1882, SEQ ID NO:1883, SEQ ID NO:1884, SEQ ID NO:1885, SEQ ID NO:1886, SEQ ID NO:1887, SEQ ID NO:1888, SEQ ID NO:1889, SEQ ID NO:1890, SEQ ID NO:1891, SEQ ID NO:1892, SEQ ID NO:1893, SEQ ID NO:1894, SEQ ID NO:1895, SEQ ID NO:1896, SEQ ID NO:1897, SEQ ID NO:1898, SEQ ID NO:1899, SEQ ID NO:1900, SEQ ID NO:1901, SEQ ID NO:1902, SEQ ID NO:1903, SEQ ID NO:1904, SEQ ID NO:1905, SEQ ID NO:1906, SEQ ID NO:1907, SEQ ID NO:1908, SEQ ID NO:1909, SEQ ID NO:1910, SEQ ID NO:1911, SEQ ID NO:1912, SEQ ID NO:1913, SEQ ID NO:1914, SEQ ID NO:1915, SEQ ID NO:1916, SEQ ID NO:1917, SEQ ID NO:1918, SEQ ID NO:1919, SEQ ID NO:1920, SEQ ID NO:1921, SEQ ID NO:1922, SEQ ID NO:1923, SEQ ID NO:1924, SEQ ID NO:1925, SEQ ID NO:1926, SEQ ID NO:1927, SEQ ID NO:1928, SEQ ID NO:1929, SEQ ID NO:1930, SEQ ID NO:1931, SEQ ID NO:1932, SEQ ID NO:1933, SEQ ID NO:1934, SEQ ID NO:1935, SEQ ID NO:1936, SEQ ID NO:1937, SEQ ID NO:1938, SEQ ID NO:1939, SEQ ID NO:1940, SEQ ID NO:1941, SEQ ID NO:1942, SEQ ID NO:1943, SEQ ID NO:1944, SEQ ID NO:1945, SEQ ID NO:1946, SEQ ID NO:1947, SEQ ID NO:1948, SEQ ID NO:1949, SEQ ID NO:1950, SEQ ID NO:1951, SEQ ID NO:1952, SEQ ID NO:1953, SEQ ID NO:1954, SEQ ID NO:1955, SEQ ID NO:1956, SEQ ID NO:1957, SEQ ID NO:1958, SEQ ID NO:1959, SEQ ID NO:1960, SEQ ID NO:1961, SEQ ID NO:1962, SEQ ID NO:1963, SEQ ID NO:1964, SEQ ID NO:1965, SEQ ID NO:1966, SEQ ID NO:1967, SEQ ID NO:1968, SEQ ID NO:1969, SEQ ID NO:1970, SEQ ID NO:1971, SEQ ID NO:1972, SEQ ID NO:1973, SEQ ID NO:1974, SEQ ID NO:1975, SEQ ID NO:1976, SEQ ID NO:1977, SEQ ID NO:1978, SEQ ID NO:1979, SEQ ID NO:1980, SEQ ID NO:1981, SEQ ID NO:1982, SEQ ID NO:1983, SEQ ID NO:1984, SEQ ID NO:1985, SEQ ID NO:1986, SEQ ID NO:1987, SEQ ID NO:1988, SEQ ID NO:1989, SEQ ID NO:1990, SEQ ID NO:1991, SEQ ID NO:1992, SEQ ID NO:1993, SEQ ID NO:1994, SEQ ID NO:1995, SEQ ID NO:1996, SEQ ID NO:1997, SEQ ID NO:1998, SEQ ID NO:1999, SEQ ID NO:2000, SEQ ID NO:2001, SEQ ID NO:2002, SEQ ID NO:2003, SEQ ID NO:2004, SEQ ID NO:2005, SEQ ID NO:2006, SEQ ID NO:2007, SEQ ID NO:2008, SEQ ID NO:2009, SEQ ID NO:2010, SEQ ID NO:2011, SEQ ID NO:2012, SEQ ID NO:2013, SEQ ID NO:2014, SEQ ID NO:2015, SEQ ID NO:2016, SEQ ID NO:2017, SEQ ID NO:2018, SEQ ID NO:2019, SEQ ID NO:2020, SEQ ID NO:2021, SEQ ID NO:2022, SEQ ID NO:2023, SEQ ID NO:2024, SEQ ID NO:2025, SEQ ID NO:2026, SEQ ID NO:2027, SEQ ID NO:2028, SEQ ID NO:2029, SEQ ID NO:2030, SEQ ID NO:2031, SEQ ID NO:2032, SEQ ID NO:2033, SEQ ID NO:2034, SEQ ID NO:2035, SEQ ID NO:2036, SEQ ID NO:2037, SEQ ID NO:2038, SEQ ID NO:2039, SEQ ID NO:2040, SEQ ID NO:2041, SEQ ID NO:2042, SEQ ID NO:2043, SEQ ID NO:2044, SEQ ID NO:2045, SEQ ID NO:2046, SEQ ID NO:2047, SEQ ID NO:2048, SEQ ID NO:2049, SEQ ID NO:2050, SEQ ID NO:2051, SEQ ID NO:2052, SEQ ID NO:2053, SEQ ID NO:2054, SEQ ID NO:2055, SEQ ID NO:2056, SEQ ID NO:2057, SEQ ID NO:2058, SEQ ID NO:2059, SEQ ID NO:2060, SEQ ID NO:2061, SEQ ID NO:2062, SEQ ID NO:2063, SEQ ID NO:2064, SEQ ID NO:2065, SEQ ID NO:2066, SEQ ID NO:2067, SEQ ID NO:2068, SEQ ID NO:2069, SEQ ID NO:2070, SEQ ID NO:2071, SEQ ID NO:2072, SEQ ID NO:2073, SEQ ID NO:2074, SEQ ID NO:2075, SEQ ID NO:2076, SEQ ID NO:2077, SEQ ID NO:2078, SEQ ID NO:2079, SEQ ID NO:2080, SEQ ID NO:2081, SEQ ID NO:2082, SEQ ID NO:2083, SEQ ID NO:2084, SEQ ID NO:2085, SEQ ID NO:2086, SEQ ID NO:2087, SEQ ID NO:2088, SEQ ID NO:2089, SEQ ID NO:2090, SEQ ID NO:2091, SEQ ID NO:2092, SEQ ID NO:2093, SEQ ID NO:2094, SEQ ID NO:2095, SEQ ID NO:2096, SEQ ID NO:2097, SEQ ID NO:2098, SEQ ID NO:2099, SEQ ID NO:2100, SEQ ID NO:2101, SEQ ID NO:2102, SEQ ID NO:2103, SEQ ID NO:2104, and SEQ ID NO:2105.

30. The isolated peptide or polypeptide of claim 1, wherein said at least a first isolated coding region comprises an amino acid sequence selected from the group consisting of SEQ SEQ ID NO:2106, SEQ ID NO:2107, SEQ ID NO:2108, SEQ ID NO:2109, SEQ ID NO:2110, SEQ ID NO:2111, SEQ ID NO:2112, SEQ ID NO:2113, SEQ ID NO:2114, SEQ ID NO:2115, SEQ ID NO:2116, SEQ ID NO:2117, SEQ ID NO:2118, SEQ ID NO:2119, SEQ ID NO:2120, SEQ ID NO:2121, SEQ ID NO:2122, SEQ ID NO:2123, SEQ ID NO:2124, SEQ ID NO:2125, SEQ ID NO:2126, SEQ ID NO:2127, SEQ ID NO:2128, SEQ ID NO:2129, SEQ ID NO:2130, SEQ ID NO:2131, SEQ ID NO:2132, SEQ ID NO:2133, SEQ ID NO:2134, SEQ ID NO:2135, SEQ ID NO:2136, SEQ ID NO:2137, SEQ ID NO:2138, SEQ ID NO:2139, SEQ ID NO:2140, SEQ ID NO:2141, SEQ ID NO:2142, SEQ ID NO:2143, SEQ ID NO:2144, SEQ ID NO:2145, SEQ ID NO:2146, SEQ ID NO:2147, SEQ ID NO:2148, SEQ ID NO:2149, SEQ ID NO:2150, SEQ ID NO:2151, SEQ ID NO:2152, SEQ ID NO:2153, SEQ ID NO:2154, SEQ ID NO:2155, SEQ ID NO:2156, SEQ ID NO:2157, SEQ ID NO:2158, SEQ ID NO:2159, SEQ ID NO:2160, SEQ ID NO:2161, SEQ ID NO:2162, SEQ ID NO:2163, SEQ ID NO:2164, SEQ ID NO:2165, SEQ ID NO:2166, SEQ ID NO:2167, SEQ ID NO:2168, SEQ ID NO:2169, SEQ ID NO:2170, SEQ ID NO:2171, SEQ ID NO:2172, SEQ ID NO:2173, SEQ ID NO:2174, SEQ ID NO:2175, SEQ ID NO:2176, SEQ ID NO:2177, SEQ ID NO:2178, SEQ ID NO:2179, SEQ ID NO:2180, SEQ ID NO:2181, SEQ ID NO:2182, SEQ ID NO:2183, SEQ ID NO:2184, SEQ ID NO:2185, SEQ ID NO:2186, SEQ ID NO:2187, SEQ ID NO:2188, SEQ ID NO:2189, SEQ ID NO:2190, SEQ ID NO:2191, SEQ ID NO:2192, SEQ ID NO:2193, SEQ ID NO:2194, SEQ ID NO:2195, SEQ ID NO:2196, SEQ ID NO:2197, SEQ ID NO:2198, SEQ ID NO:2199, SEQ ID NO:2200, SEQ ID NO:2201, SEQ ID NO:2202, SEQ ID NO:2203, SEQ ID NO:2204, SEQ ID NO:2205, SEQ ID NO:2206, SEQ ID NO:2207, SEQ ID NO:2208, SEQ ID NO:2209, SEQ ID NO:2210, SEQ ID NO:2211, SEQ ID NO:2212, SEQ ID NO:2213, SEQ ID NO:2214, SEQ ID NO:2215, SEQ ID NO:2216, SEQ ID NO:2217, SEQ ID NO:2218, SEQ ID NO:2219, SEQ ID NO:2220, SEQ ID NO:2221, SEQ ID NO:2222, SEQ ID NO:2223, SEQ ID NO:2224, SEQ ID NO:2225, SEQ ID NO:2226, SEQ ID NO:2227, SEQ ID NO:2228, SEQ ID NO:2229, SEQ ID NO:2230, SEQ ID NO:2231, SEQ ID NO:2232, SEQ ID NO:2233, SEQ ID NO:2234, SEQ ID NO:2235, SEQ ID NO:2236, SEQ ID NO:2237, SEQ ID NO:2238, SEQ ID NO:2239, SEQ ID NO:2240, SEQ ID NO:2241, SEQ ID NO:2242, SEQ ID NO:2243, SEQ ID NO:2244, SEQ ID NO:2245, SEQ ID NO:2246, SEQ ID NO:2247, SEQ ID NO:2248, SEQ ID NO:2249, SEQ ID NO:2250, SEQ ID NO:2251, SEQ ID NO:2252, SEQ ID NO:2253, SEQ ID NO:2254, SEQ ID NO:2255, SEQ ID NO:2256, SEQ ID NO:2257, SEQ ID NO:2258, SEQ ID NO:2259, SEQ ID NO:2260, SEQ ID NO:2261, SEQ ID NO:2262, SEQ ID NO:2263, SEQ ID NO:2264, SEQ ID NO:2265, SEQ ID NO:2266, SEQ ID NO:2267, SEQ ID NO:2268, SEQ ID NO:2269, SEQ ID NO:2270, SEQ ID NO:2271, SEQ ID NO:2272, SEQ ID NO:2273, SEQ ID NO:2274, SEQ ID NO:2275, SEQ ID NO:2276, SEQ ID NO:2277, SEQ ID NO:2278, SEQ ID NO:2279, SEQ ID NO:2280, SEQ ID NO:2281, SEQ ID NO:2282, SEQ ID NO:2283, SEQ ID NO:2284, SEQ ID NO:2285, SEQ ID NO:2286, SEQ ID NO:2287, SEQ ID NO:2288, SEQ ID NO:2289, SEQ ID NO:2290, SEQ ID NO:2291, SEQ ID NO:2292, SEQ ID NO:2293, SEQ ID NO:2294, SEQ ID NO:2295, SEQ ID NO:2296, SEQ ID NO:2297, SEQ ID NO:2298, SEQ ID NO:2299, SEQ ID NO:2300, SEQ ID NO:2301, SEQ ID NO:2302, SEQ ID NO:2303, SEQ ID NO:2304, SEQ ID NO:2305, SEQ ID NO:2306, SEQ ID NO:2307, SEQ ID NO:2308, SEQ ID NO:2309, SEQ ID NO:2310, SEQ ID NO:2311, SEQ ID NO:2312, SEQ ID NO:2313, SEQ ID NO:2314, SEQ ID NO:2315, SEQ ID NO:2316, SEQ ID NO:2317, SEQ ID NO:2318, SEQ ID NO:2319, SEQ ID NO:2320, SEQ ID NO:2321, SEQ ID NO:2322, SEQ ID NO:2323, SEQ ID NO:2324, SEQ ID NO:2325, SEQ ID NO:2326, SEQ ID NO:2327, SEQ ID NO:2328, SEQ ID NO:2329, SEQ ID NO:2330, SEQ ID NO:2331, SEQ ID NO:2332, SEQ ID NO:2333, SEQ ID NO:2334, SEQ ID NO:2335, SEQ ID NO:2336, SEQ ID NO:2337, SEQ ID NO:2338, SEQ ID NO:2339, SEQ ID NO:2340, SEQ ID NO:2341, SEQ ID NO:2342, SEQ ID NO:2343, SEQ ID NO:2344, SEQ ID NO:2345, SEQ ID NO:2346, SEQ ID NO:2347, SEQ ID NO:2348, SEQ ID NO:2349, SEQ ID NO:2350, SEQ ID NO:2351, SEQ ID NO:2352, SEQ ID NO:2353, SEQ ID NO:2354, SEQ ID NO:2355, SEQ ID NO:2356, SEQ ID NO:2357, SEQ ID NO:2358, SEQ ID NO:2359, SEQ ID NO:2360, SEQ ID NO:2361, SEQ ID NO:2362, SEQ ID NO:2363, SEQ ID NO:2364, SEQ ID NO:2365, SEQ ID NO:2366, SEQ ID NO:2367, SEQ ID NO:2368, SEQ ID NO:2369, SEQ ID NO:2370, SEQ ID NO:2371, SEQ ID NO:2372, SEQ ID NO:2373, SEQ ID NO:2374, and SEQ ID NO:2375.

31. The isolated peptide or polypeptide of claim 1, wherein said at least a first isolated coding region comprises an amino acid sequence selected from the group consisting of SEQ ID NO:2376, SEQ ID NO:2377, SEQ ID NO:2378, SEQ ID NO:2379, SEQ ID NO:2380, SEQ ID NO:2381, SEQ ID NO:2382, SEQ ID NO:2383, SEQ ID NO:2384, SEQ ID NO:2385, SEQ ID NO:2386, SEQ ID NO:2387, SEQ ID NO:2388, SEQ ID NO:2389, SEQ ID NO:2390, SEQ ID NO:2391, SEQ ID NO:2392, SEQ ID NO:2393, SEQ ID NO:2394, SEQ ID NO:2395, SEQ ID NO:2396, SEQ ID NO:2397, SEQ ID NO:2398, SEQ ID NO:2399, SEQ ID NO:2400, SEQ ID NO:2401, SEQ ID NO:2402, SEQ ID NO:2403, SEQ ID NO:2404, SEQ ID NO:2405, SEQ ID NO:2406, SEQ ID NO:2407, SEQ ID NO:2408, SEQ ID NO:2409, SEQ ID NO:2410, SEQ ID NO:2411, SEQ ID NO:2412, SEQ ID NO:2413, SEQ ID NO:2414, SEQ ID NO:2415, SEQ ID NO:2416, SEQ ID NO:2417, SEQ ID NO:2418, SEQ ID NO:2419, SEQ ID NO:2420, SEQ ID NO:2421, SEQ ID NO:2422, SEQ ID NO:2423, SEQ ID NO:2424, SEQ ID NO:2425, SEQ ID NO:2426, SEQ ID NO:2427, SEQ ID NO:2428, SEQ ID NO:2429, SEQ ID NO:2430, SEQ ID NO:2431, SEQ ID NO:2432, SEQ ID NO:2433, SEQ ID NO:2434, SEQ ID NO:2435, SEQ ID NO:2436, SEQ ID NO:2437, SEQ ID NO:2438, SEQ ID NO:2439, SEQ ID NO:2440, SEQ ID NO:2441, SEQ ID NO:2442, SEQ ID NO:2443, SEQ ID NO:2444, SEQ ID NO:2445, SEQ ID NO:2446, SEQ ID NO:2447, SEQ ID NO:2448, SEQ ID NO:2449, SEQ ID NO:2450, SEQ ID NO:2451, SEQ ID NO:2452, SEQ ID NO:2453, SEQ ID NO:2454, SEQ ID NO:2455, SEQ ID NO:2456, SEQ ID NO:2457, SEQ ID NO:2458, SEQ ID NO:2459, SEQ ID NO:2460, SEQ ID NO:2461, SEQ ID NO:2462, SEQ ID NO:2463, SEQ ID NO:2464, SEQ ID NO:2465, SEQ ID NO:2466, SEQ ID NO:2467, SEQ ID NO:2468, SEQ ID NO:2469, SEQ ID NO:2470, SEQ ID NO:2471, SEQ ID NO:2472, SEQ ID NO:2473, SEQ ID NO:2474, SEQ ID NO:2475, SEQ ID NO:2476, SEQ ID NO:2477, SEQ ID NO:2478, SEQ ID NO:2479, SEQ ID NO:2480, SEQ ID NO:2481, SEQ ID NO:2482, SEQ ID NO:2483, SEQ ID NO:2484, SEQ ID NO:2485, SEQ ID NO:2486, SEQ ID NO:2487, SEQ ID NO:2488, SEQ ID NO:2489, SEQ ID NO:2490, SEQ ID NO:2491, SEQ ID NO:2492, SEQ ID NO:2493, SEQ ID NO:2494, SEQ ID NO:2495, SEQ ID NO:2496, SEQ ID NO:2497, SEQ ID NO:2498, SEQ ID NO:2499, SEQ ID NO:2500, SEQ ID NO:2501, SEQ ID NO:2502, SEQ ID NO:2503, SEQ ID NO:2504, SEQ ID NO:2505, SEQ ID NO:2506, SEQ ID NO:2507, SEQ ID NO:2508, SEQ ID NO:2509, SEQ ID NO:2510, SEQ ID NO:2511, SEQ ID NO:2512, SEQ ID NO:2513, SEQ ID NO:2514, SEQ ID NO:2515, SEQ ID NO:2516, SEQ ID NO:2517, SEQ ID NO:2518 SEQ ID NO:2519, SEQ ID NO:2520, SEQ ID NO:2521, SEQ ID NO:2522, SEQ ID NO:2523, SEQ ID NO:2524, SEQ ID NO:2525, SEQ ID NO:2526, SEQ ID NO:2527, SEQ ID NO:2528, SEQ ID NO:2529, SEQ ID NO:2530, SEQ ID NO:2531, and SEQ ID NO:2532.

32. The isolated peptide or polypeptide of claim 1, wherein said amino acid sequence consists essentially of the sequence of any one of SEQ ID NO:669 to SEQ ID NO:2532.

33. The isolated peptide or polypeptide of claim 32, wherein said amino acid sequence consists of the sequence of any one of SEQ ID NO:669 to SEQ ID NO:2532.

34. The isolated peptide or polypeptide of claim 1, wherein said at least a first coding region comprises an amino acid sequence that is encoded by an at least 100 contiguous nucleotide sequence from any one of SEQ ID NO:1 to SEQ ID NO:668.

35. The isolated peptide or polypeptide of claim 34, wherein said at least a first coding region comprises an amino acid sequence that is encoded by an at least 200 contiguous nucleotide sequence from any one of SEQ ID NO:1 to SEQ ID NO:668.

36. The isolated peptide or polypeptide of claim 35, wherein said at least a first coding region comprises an amino acid sequence that is encoded by a nucleic acid segment comprising the nucleotide sequence of any one of SEQ ID NO:1 to SEQ ID NO:668.

37. The isolated peptide or polypeptide of claim 36, wherein said at least a first coding region comprises an amino acid sequence that is encoded by a nucleic acid segment that consists essentially of the nucleotide sequence of any one of SEQ ID NO:1 to SEQ ID NO:668.

38. The isolated peptide or polypeptide of claim 37, wherein said at least a first coding region comprises an amino acid sequence that is encoded by a nucleic acid segment that consists of the nucleotide sequence of any one of SEQ ID NO:1 to SEQ ID NO:668.

39. A composition comprising the isolated peptide or polypeptide of claim 1.

40. The composition according to claim 39, further comprising a pharmaceutically-acceptable diluent.

41. An isolated polynucleotide, comprising at least a first nucleic acid segment that (a) encodes the isolated peptide or polypeptide of claim 1; or (b) comprises an at least 100 contiguous nucleotide sequence from any one of SEQ ID NO:1 to SEQ ID NO:668.

42. The isolated polynucleotide of claim 41, comprising at least a first nucleic acid segment that encodes the isolated peptide or polypeptide of claim 1.

43. The isolated polynucleotide of claim 41, comprising at least a first nucleic acid segment that comprises an at least 25 contiguous nucleotide sequence from any one of SEQ ID NO:1 to SEQ ID NO:668.

44. The isolated polynucleotide of claim 43, comprising at least a first nucleic acid segment that comprises an at least 50 contiguous nucleotide sequence from any one of SEQ ID NO:1 to SEQ ID NO:668.

45. The isolated polynucleotide of claim 44, comprising at least a first nucleic acid segment that comprises an at least 75 contiguous nucleotide sequence from any one of SEQ ID NO:1 to SEQ ID NO:668.

46. The isolated polynucleotide of claim 45, comprising at least a first nucleic acid segment that comprises an at least 100 contiguous nucleotide sequence from any one of SEQ ID NO:1 to SEQ ID NO:668.

47. The isolated polynucleotide of claim 46, wherein said at least a first nucleic acid segment comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110 , SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192 SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, and SEQ ID NO:278.

48. The isolated polynucleotide of claim 46, wherein said at least a first nucleic acid segment comprises a nucleotide sequence selected from the group consisting of SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342 , SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:4083, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, and SEQ ID NO:436.

49. The isolated polynucleotide of claim 46, wherein said at least a first nucleic acid segment comprises a nucleotide sequence selected from the group consisting of SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:503, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, SEQ ID NO:524, SEQ ID NO:525, SEQ ID NO:526, SEQ ID NO:527, and SEQ ID NO:528.

50. The isolated polynucleotide of claim 46, wherein said at least a first nucleic acid segment comprises a nucleotide sequence selected from the group consisting of SEQ ID NO:529, SEQ ID NO:530, SEQ ID NO:531, SEQ ID NO:532, SEQ ID NO:533, SEQ ID NO:534, SEQ ID NO:535, SEQ ID NO:536, SEQ ID NO:537, SEQ ID NO:538, SEQ ID NO:539, SEQ ID NO:540, SEQ ID NO:541, SEQ ID NO:542, SEQ ID NO:543, SEQ ID NO:544, SEQ ID NO:545, SEQ ID NO:546, SEQ ID NO:547, SEQ ID NO:548, SEQ ID NO:549, SEQ ID NO:550, SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, and SEQ ID NO:610.

51. The isolated polynucleotide of claim 46, wherein said at least a first nucleic acid segment comprises a nucleotide sequence selected from the group consisting of SEQ ID NO:611, SEQ ID NO:612, SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:627, SEQ ID NO:628, SEQ ID NO:629, SEQ ID NO:630, SEQ ID NO:631, SEQ ID NO:632, SEQ ID NO:633, SEQ ID NO:634, SEQ ID NO:635, SEQ ID NO:636, SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, SEQ ID
NO:662, SEQ ID NO:663, and SEQ ID NO:664.

52. The isolated polynucleotide of claim 41, wherein said at least a first nucleic acid segment is operably positioned under the control of at least a first heterologous promoter.

53. The isolated polynucleotide of claim 41, further comprising at least a second nucleic acid segment that encodes at least a second isolated peptide or polypeptide.

54. The isolated polynucleotide of claim 53, wherein said polynucleotide comprises said at least a first isolated nucleic acid segment operably attached, in frame, to said at least a second isolated nucleic acid segment, said polynucleotide encoding a fusion protein in which said first isolated peptide or polypeptide is linked to said second isolated peptide or polypeptide.

55. The isolated polynucleotide of claim 54, wherein said at least a second isolated nucleic acid segment encodes: (a) an adjuvant peptide or polypeptide, (b) an immunostimulant peptide or polypeptide, or (c) at least a second distinct peptide or polypeptide that comprises an amino acid sequence that is at least about 90% identical to the amino acid sequence of any one of SEQ ID NO:669 to SEQ ID NO:2532.

56. The isolated polynucleotide of claim 41, wherein said at least a first nucleic acid segment is comprised within a vector.

57. The isolated polynucleotide of claim 41, wherein said polynucleotide is comprised within a host cell.

58. A vector comprising the isolated polynucleotide of claim 41.

59. The vector of claim 58, wherein said vector is a plasmid or viral vector.

60. An isolated host cell comprising the isolated polynucleotide of claim 41, or the vector of claim 58.

61. The isolated host cell of claim 60, wherein said host cell is an isolated human blood or bone marrow cell.

62. The isolated host cell of claim 61, wherein said human blood or bone marrow cell is isolated from a patient having, suspected of having, or at risk for developing a hematological malignancy selected from the group consisting of Hodgkin's lymphoma, follicular lymphoma, B cell-type non-Hodgkin's lymphoma, T cell-type non-Hodgkin's lymphoma, lymphoma, and chronic lymphocytic leukemia.

63. An isolated antigen-presenting cell that expresses the peptide or polypeptide of claim 1, wherein said cell is obtained from a patient having, suspected of having, or at risk for developing a hematological malignancy selected from the group consisting of leukemia and lymphoma.

64. A plurality of isolated T cells that specifically react with the peptide or polypeptide of claim 1, wherein said cells are obtained from a patient having, suspected of having, or at risk for developing a hematological malignancy selected from the group consisting of leukemia and lymphoma.

65. The plurality of isolated T cells of claim 64, wherein said cells are stimulated or expanded by contacting said cells with the peptide or polypeptide of claim 1.

66. The plurality of isolated T cells of claim 65, wherein said cells are cloned prior to expansion.

67. The plurality of isolated T cells of claim 64, wherein said cells are obtained from bone marrow, a bone marrow fraction, peripheral blood, or a peripheral blood fraction of a patient having, suspected of having, or at risk for developing a hematological malignancy selected from the group consisting of leukemia and lymphoma.

68. A composition comprising the isolated polynucleotide of claim 41, the vector of claim 58, the isolated host cell of claim 60, the isolated antigen-presenting cell of claim 63, or the plurality of isolated T cells of claim 64.

69. The composition of claim 68, further comprising a pharmaceutically-acceptable diluent.

70. The composition of claim 69, wherein said composition is formulated for parenteral, intravenous, intraarterial, intraosseus, intrathecal, intraperitoneal, subcutaneous, intranasal, transdermal, sublingual, or oral administration.

71. The composition of claim 71, further comprising at least a first immunostimulant or at least a first adjuvant.

72. The composition of claim 71, further comprising at least a first immunostimulant or at least a first adjuvant selected from the group consisting of Montanide ISA50, Seppic Montanide ISA720, a cytokine, a microsphere, a dimethyl dioctadecyl ammonium bromide adjuvant, AS-1, AS-2, Ribi Adjuvant, QS21, saponin, microfluidized Syntex adjuvant, MV, ddMV, an immune stimulating complex and an inactivated toxin.

73. The composition of claim 68, further comprising at least a first detection reagent.

74. The composition of claim 68, further comprising at least a first therapeutic agent.

75. The composition of claim 68, further comprising at least a first anti-cancer agent used in the treatment of Hodgkin's lymphoma, follicular lymphoma, B cell-type non-Hodgkin's lymphoma, T cell-type non-Hodgkin's lymphoma, lymphoma, or chronic lymphocytic leukemia.

76. A composition comprising: (a) at least a first isolated peptide or polypeptide comprising at least a first coding region that comprises an amino acid sequence that is at least about 90% identical to the amino acid sequence of any one of SEQ ID NO:669 to SEQ ID NO:2532; and (b) at least a second distinct isolated peptide or polypeptide comprising at least a first coding region that comprises an amino acid sequence that is at least about 90% identical to the amino acid sequence of any one of SEQ ID NO:669 to SEQ ID NO:2532.

77. The composition of claim 76, further comprising at least a third distinct isolated peptide or polypeptide comprising at least a first coding region that comprises an amino acid sequence that is at least about 90% identical to the amino acid sequence of any one of SEQ ID NO:669 to SEQ ID NO:2532.

78. A composition comprising: (a) at least a first isolated polynucleotide that encodes a first isolated peptide or polypeptide comprising at least a first coding region that comprises an amino acid sequence that is at least about 90% identical to the amino acid sequence of any one of SEQ ID NO:669 to SEQ ID NO:2532; and (b) at least a second distinct isolated polynucleotide that encodes a second distinct isolated peptide or polypeptide comprising at least a first coding region that comprises an amino acid sequence that is at least about 90% identical to the amino acid sequence of any one of SEQ ID NO:669 to SEQ ID NO:2532.

79. The composition of claim 78, further comprising at least a third distinct isolated polynucleotide that encodes a third distinct isolated peptide or polypeptide comprising at least a first coding region that comprises an amino acid sequence that is at least about 90% identical to the amino acid sequence of any one of SEQ ID NO:669 to SEQ ID NO:2532.

80. A composition comprising: (a) at least a first isolated polynucleotide that encodes a first isolated peptide or polypeptide comprising at least a first coding region that comprises an amino acid sequence that is at least about 90% identical to the amino acid sequence of any one of SEQ ID NO:669 to SEQ ID NO:2532; and (b) at least a second distinct isolated peptide or polypeptide comprising at least a first coding region that comprises an amino acid sequence that is at least about 90% identical to the amino acid sequence of any one of SEQ ID NO:669 to SEQ ID NO:2532.

81. A hybridoma cell line that produces a monoclonal antibody having immunospecificity for the peptide or polypeptide of claim 1.

82. An isolated antibody, or an antigen-binding fragment thereof, that has immunospecificity for a peptide or polypeptide consisting essentially of the sequence of any one of SEQ ID NO:669 to SEQ ID NO:2532.

83. The antibody of claim 82, wherein said antigen-binding fragment comprises a light chain variable region, a heavy-chain variable region, a Fab fragment, a F(ab)₂ fragment, an Fv fragment, an scFv fragment, or an antigen-binding fragment of said antibody.

84. A kit comprising: (a) the peptide or polypeptide of claim 1, the polynucleotide of claim 41, the vector of claim 58, the host cell of claim 60, the antigen presenting cell of claim 63, the plurality of T cells of claim 64, the composition of claim 76, the composition of claim 78, the composition of claim 80, the hybridoma cell line of claim 81, or the antibody or antigen binding fragment of claim 82; and (b) instructions for using said kit in the diagnosis, detection, or treatment of at least a first hematological malignancy selected from the group consisting of Hodgkin's lymphoma, follicular lymphoma, B cell-type non-Hodgkin's lymphoma, T cell-type non-Hodgkin's lymphoma, lymphoma, and chronic lymphocytic leukemia.

85. The kit of claim 84, wherein said kit further comprises a therapeutically effective amount of at least a second anti-cancer agent.

86. A method of generating an immune response in an animal, comprising providing to said animal an effective amount of the peptide or polypeptide of claim 1, the polynucleotide of claim 41, the vector of claim 58, the host cell of claim 60, the antigen presenting cell of claim 63, or the plurality of T cells of claim 64.

87. A method of generating a T-cell response in an animal, comprising providing to said animal an effective amount of the peptide or polypeptide of claim 1, the polynucleotide of claim 41, the vector of claim 58, the host cell of claim 60, or the antigen presenting cell of claim 63.

88. The method of claim 86 or 87, wherein said animal is a human having, suspected of having, or at risk for developing a hematological malignancy selected from the group consisting of Hodgkin's lymphoma, follicular lymphoma, B cell-type non-Hodgkin's lymphoma, T cell-type non-Hodgkin's lymphoma, lymphoma, and chronic lymphocytic leukemia.

89. A method of assessing the risk of a human patient in developing a hematological malignancy selected from the group consisting of Hodgkin's lymphoma, follicular lymphoma, B cell-type non-Hodgkin's lymphoma, T cell-type non-Hodgkin's lymphoma, and lymphoma; said method comprising detecting the presence of the peptide or polypeptide of claim 1, the polynucleotide of claim 41, or the antibody of claim 82, in a clinical sample obtained from said patient, wherein an increased level of said peptide, polypeptide, polynucleotide, or antibody relative to an unaffected human is indicative of an increased risk for developing said hematological malignancy.

90. A method of detecting a Hodgkin's lymphoma hematological malignancy-related polypeptide, polynucleotide, antibody, or an antigen binding fragment thereof, or in a biological sample or an animal cell said method comprising, contacting a sample or a cell suspected of containing a Hodgkin's lymphoma hematological malignancy with: (a) a labeled peptide or polypeptide according to claim 12 or 27, (b) a labeled antibody, or a labeled antigen binding fragment thereof, that is immunospecific for the peptide or polypeptide of claim 12 or 27, (c) a labeled polynucleotide according to claim 47, or (d) a labeled polynucleotide that comprises the sequence of any of one SEQ ID NO:665 to SEQ ID NO:668, or SEQ ID NO:2533 through SEQ ID NO:9597, under conditions effective and for a time sufficient to allow immunocomplexes or specific hybridization complexes to form, wherein the presence of labeled immunocomplexes or labeled hybridization complexes is indicative of the presence of said Hodgkin's lymphoma hematological malignancy-related polypeptide, polynucleotide, antibody, or antigen binding fragment in said sample or said cell.

91. A method of detecting a follicular lymphoma hematological malignancy-related polypeptide, polynucleotide, antibody, or an antigen binding fragment thereof, or in a biological sample or an animal cell said method comprising, contacting a sample or a cell suspected of containing a follicular lymphoma hematological malignancy with: (a) a labeled peptide or polypeptide according to claim 15 or 28, (b) a labeled antibody, or a labeled antigen binding fragment thereof, that is immunospecific for the peptide or polypeptide of claim 15 or 28, (c) a labeled polynucleotide according to claim 48, or (d) a labeled polynucleotide that comprises the sequence of any of one SEQ ID NO:665 to SEQ ID NO:668, or SEQ ID NO:2533 through SEQ ID NO:9597, under conditions effective and for a time sufficient to allow immunocomplexes or specific hybridization complexes to form, wherein the presence of labeled immunocomplexes or labeled hybridization complexes is indicative of the presence of said follicular lymphoma hematological malignancy-related polypeptide, polynucleotide, antibody, or antigen binding fragment in said sample or said cell.

92. A method of detecting a B cell non-Hodgkin's lymphoma hematological malignancy-related polypeptide, polynucleotide, antibody, or an antigen binding fragment thereof, or in a biological sample or an animal cell said method comprising, contacting a sample or a cell suspected of containing a B cell non-Hodgkin's lymphoma hematological malignancy with: (a) a labeled peptide or polypeptide according to claim 18 or 29, (b) a labeled antibody, or a labeled antigen binding fragment thereof, that is immunospecific for the peptide or polypeptide of claim 18 or 29, (c) a labeled polynucleotide according to claim 49, or (d) a labeled polynucleotide that comprises the sequence of any of one SEQ ID NO:665 to SEQ ID NO:668, or SEQ ID NO:2533 through SEQ ID NO:9597, under conditions effective and for a time sufficient to allow immunocomplexes or specific hybridization complexes to form, wherein the presence of labeled immunocomplexes or labeled hybridization complexes is indicative of the presence of said B cell non-Hodgkin's lymphoma hematological malignancy-related polypeptide, polynucleotide, antibody, or antigen binding fragment in said sample or said cell.

93. A method of detecting a T cell non-Hodgkin's lymphoma hematological malignancy-related polypeptide, polynucleotide, antibody, or an antigen binding fragment thereof, or in a biological sample or an animal cell said method comprising, contacting a sample or a cell suspected of containing a T cell non-Hodgkin's lymphoma hematological malignancy with: (a) a labeled peptide or polypeptide according to claim 21 or 30, (b) a labeled antibody, or a labeled antigen binding fragment thereof, that is immunospecific for the peptide or polypeptide of claim 21 or 30, (c) a labeled polynucleotide according to claim 50, or (d) a labeled polynucleotide that comprises the sequence of any of one SEQ ID NO:665 to SEQ ID NO:668, or SEQ ID NO:2533 through SEQ ID NO:9597, under conditions effective and for a time sufficient to allow immunocomplexes or specific hybridization complexes to form, wherein the presence of labeled immunocomplexes or labeled hybridization complexes is indicative of the presence of said T cell non-Hodgkin's lymphoma hematological malignancy-related polypeptide, polynucleotide, antibody, or antigen binding fragment in said sample or said cell.

94. A method of detecting a lymphoma-related malignancy polypeptide, polynucleotide, antibody, or an antigen binding fragment thereof, or in a biological sample or an animal cell said method comprising, contacting a sample or a cell suspected of containing a lymphoma-related malignancy with: (a) a labeled peptide or polypeptide according to claim 24 or 31, (b) a labeled antibody, or a labeled antigen binding fragment thereof, that is immunospecific for the peptide or polypeptide of claim 24 or 31, (c) a labeled polynucleotide according to claim 51, or (d) a labeled polynucleotide that comprises the sequence of any of one SEQ ID NO:665 to SEQ ID NO:668, or SEQ ID NO:2533 through SEQ ID NO:9597, under conditions effective and for a time sufficient to allow immunocomplexes or specific hybridization complexes to form, wherein the presence of labeled immunocomplexes or labeled hybridization complexes is indicative of the presence of said lymphoma-related malignancy-related polypeptide, polynucleotide, antibody, or antigen binding fragment in said sample or said cell.

95. A method for detecting antibodies specific for a hematological malignancy-related peptide or polypeptide in a biological sample, said method comprising the steps of:
(a) contacting a first biological sample suspected of containing said antibodies with the peptide or polypeptide of claim 1;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.

96. A method for detecting antibodies specific for a Hodgkin's lymphoma-specific hematological malignancy-related peptide or polypeptide in a biological sample, said method comprising the steps of:
(a) contacting a first biological sample suspected of containing said antibodies with the peptide or polypeptide of claim 12 or 27;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.

97. A method for detecting antibodies specific for a follicular lymphoma-specific hematological malignancy-related peptide or polypeptide in a biological sample, said method comprising the steps of:
(a) contacting a first biological sample suspected of containing said antibodies with the peptide or polypeptide of claim 15 or 28;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.

98. A method for detecting antibodies specific for a B cell non-Hodgkin's lymphoma-specific hematological malignancy-related peptide or polypeptide in a biological sample, said method comprising the steps of:
(a) contacting a first biological sample suspected of containing said antibodies with the peptide or polypeptide of claim 18 or 29;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.

99. A method for detecting antibodies specific for a T cell non-Hodgkin's lymphoma-specific hematological malignancy-related peptide or polypeptide in a biological sample, said method comprising the steps of:
(a) contacting a first biological sample suspected of containing said antibodies with the peptide or polypeptide of claim 21 or 30;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.

100. A method for detecting antibodies specific for a lymphoma-specific hematological malignancy-related peptide or polypeptide in a biological sample, said method comprising the steps of:
(a) contacting a first biological sample suspected of containing said antibodies with the peptide or polypeptide of claim 24 or 31;
(b) incubating said sample under conditions effective and for a time sufficient to allow immunocomplexes to form; and
(c) detecting immunocomplexes formed between said peptide or polypeptide and antibodies in said sample that are specific for said peptide or polypeptide, wherein the presence of said immunocomplexes is indicative of the presence of said antibodies in said sample.
